(19) United States
(12) Patent Application Publication
(10) Pub. No.: US 2026/0306644 A1
(43) Pub. Date: Oct. 8, 2026

(54) ANTIBODY RECRUITING MOLECULES FOR THE TREATMENT OF CORONAVIRUS DISEASE-19

(71) Applicant: Biohaven Therapeutics Ltd., New Haven, CT (US)

(72) Inventors: Wieslaw KAZMIERSKI, Durham, NC (US); Richard PRACITTO, Wallingford, CT (US); Alexander Sergei BAYDEN, Hamden, CT (US); Lawrence G. IBEN, Bethany, CT (US); Anna BUNIN, Queens, NY (US); Anne Marie ROSSI, Clinton, CT (US)

(21) Appl. No.: 19/477,100

(22) PCT Filed: Apr. 29, 2024
(86) PCT No.: PCT/US2024/026843

§ 371 Date: Oct. 20, 2025

Related U.S. Application Data
(60) Provisional application No. 63/499,211, filed on Apr. 29, 2023.

(51) Int. Cl. A61K 47/64; A61K 40/15; A61K 47/54; A61K 47/55

(52) U.S. Cl. CPC A61K 47/64; A61K 40/15; A61K 47/545; A61K 47/55

(57) ABSTRACT

The present disclosure describes a compound or antibody recruiting molecule (ARM), or a pharmaceutically acceptable salt thereof, having one or more antibody binding moiety (ABM) that binds to one or more antibodies or fragments thereof, one or more target binding moiety (TBM) that binds the spike glycoprotein of a coronavirus (e.g., severe acute respiratory syndrome coronavirus 2), and optionally a linker moiety (L) that covalently links the one or more ABMs and the one or more TBMs. Further described are methods of making and using the compounds of the present disclosure.

### CROSS REFERENCE TO RELATED APPLICATION

This application claims priority to U.S. Provisional Application No. 63/499,211, filed Apr. 29, 2023, which is hereby incorporated by reference in its entirety.

### INCORPORATION BY REFERENCE

In compliance with 37 C.F.R. § 1.52(e)(5), the sequence information contained in electronic file name: BHL0041US-Sequence-Listing-ST26.xml, size: 201 KB, crated on: 1 Mar. 2023 using WIPO Sequence 2.1.0, is filed herewith and hereby incorporated by reference in its entirety.

### FIELD OF THE INVENTION

The present disclosure describes compounds that modulate immune activities, e.g., for treatment of coronavirus infections, such as severe acute respiratory syndrome coronavirus 2 (SARS-CoV-2) infections or Coronavirus Disease 19 (COVID-19). The present disclosure describes compounds and methods useful as enhancing activities of antibodies, e.g., monoclonal antibodies. The disclosure further describes pharmaceutically acceptable compositions comprising the described compounds and methods of using said compositions in the treatment of various coronavirus infections, such as a SARS-CoV-2 infection or COVID-19.

### BACKGROUND

The search for new therapeutic agents has been greatly aided in recent years by the discovery and development of monoclonal antibody therapeutic agents. Several of such agents elicit their therapeutic effect through the concomitant engagement of extracellular receptors of a targeted diseased cell with immune effector cells. Strategies for improving the therapeutic efficacy of antibodies, such as monoclonal antibodies (mAbs), have involved engineering antibodies (i.e. mutated amino acids, altered glycosylation) to enhance the binding to fragment crystallizable (Fc) receptors.

### SUMMARY

Many immune activities involve the recruitment of immune cells (such as, NK cells) to damaged, diseased, and/or defective cells (such as, coronavirus infected cells, including SARS-CoV-2 infected cells), tissues (such as, coronavirus infected tissue, including SARS-CoV-2 infected tissue, etc.), foreign objects and/or entities (such as, coronavirus, including SARS-CoV-2), etc. Among other things, the present disclosure provides compounds, compositions, methods, etc., that are particularly useful for triggering, generating, encouraging, and/or enhancing recruitment of immune cells to their target sites, which, among other things, can provide improved biological effects (e.g., improved therapeutic effects, lower toxicity, fewer/less severe side effects, etc.

In any aspect or embodiment described herein, the present disclosure provides technologies that can modulate activities of certain fragment crystallizable (Fc) receptors. In any aspect or embodiment described herein, the present disclosure provides compounds that can bind certain Fc receptors and compositions and methods thereof. In any aspect or embodiment described herein, the compounds and compositions of the present disclosure can trigger, generate, encourage and/or enhance one or more immune activities by enhanced recruitment of immune cells that express such Fc receptors. In any aspect or embodiment described herein, compounds of the present disclosure utilize binding moieties to enhance binding to Fc receptors. In any aspect or embodiment described herein, compounds and compositions of the present disclosure enhance the binding of antibodies, e.g., monoclonal antibodies, to Fc receptors. Among other things, such enhanced binding can provide enhanced recruitment of immune cells expressing such Fc receptors and provide improved biological results, e.g., improved therapeutic effects, lower toxicity, fewer/less severe side effects, etc.

Natural killer (NK) cells are capable of inducing cell death of various targets, e.g., diseased cells, such as virally infected cells. An aspect of the present disclosure encompasses the recognition that treatment of viral infections can be significantly improved by agents that can facilitate and/or promote of recognition of targets by NK cells. In any aspect or embodiment described herein, the present disclosure provides technologies (e.g., methods, compositions, etc.) comprising NK cells and such agents, particularly, in any aspect or embodiment described herein, the present disclosure provides technologies comprising NK cells and antibody-recruiting molecules (ARMs), which comprise (i) an antibody binding moiety (ABM) that can bind to one or more antibodies or fragments thereof, (ii) a target-binding moiety (TBM) that can bind to the cell, and optionally (iii) a linker moiety connecting the antibody binding moiety with the target binding moiety.

In any aspect or embodiment described herein, a target binding moiety (also referred to herein as a target binding terminus (TBT)) can confer specificity of an ARM to its target, e.g., a diseased cell of interest, through, e.g., binding an entity (e.g., a cell surface receptor) differentiating a target from a non-target (e.g., diseased cells from other cell types). In any aspect or embodiment described herein, the target binding moiety is a coronavirus binding agent, such as a small molecules or peptide. In any aspect or embodiment described herein, a target binding moiety binds to an antigen of a damaged, diseased, and/or defective cells (e.g., a coronavirus infected cell, such as a SARS-CoV-2 infected cell), tissues (e.g., a coronavirus infected cell, a SARS-CoV-2 infected cell, etc.), foreign objects and/or entities (e.g., coronavirus infected cell or SARS-CoV-2), etc. In any aspect or embodiment described herein, an antibody (e.g., a monoclonal antibody (mAb)) attaches to disease cells targeted for destruction. In any aspect or embodiment described herein, a target binding moiety is a coronavirus binding moiety (CBM) that binds to a coronavirus spike glycoprotein or protein (e.g., SARS-CoV-2 spike glycoprotein) that is expressed by cells infected with a coronavirus (e.g., SARS-CoV-2). In any aspect or embodiment described herein, antibody binding moiety binds to Fc portions of antibodies (e.g., IgG antibodies) which then activates immune cells, e.g., NK cells, for immune activities such as ADCC.

Among other things, ARMs can enable target-specific recruitment of antibodies, e.g., endogenous antibodies, administered antibodies, etc., through ABTs, and/or trigger, generate, encourage, and/or enhance NK-cell related immune activities, e.g., killing of target cells by NK cells. Various technologies, e.g., assays, reagents, methods, etc. are available for assessing antibody binding moieties, target binding moieties and ARMs and can be utilized in accordance with the present disclosure.

In any aspect or embodiment described herein, an antibody-recruiting molecule (ARM) comprises:
an antibody binding moiety (ABM) or an antibody binding terminus (ABT),
a target binding moiety (TBM) or target binding terminus (TBT), and
optionally a linker moiety that covalently links the ABM/ABT to the TBM/TBT, wherein the antibody binding moiety or antibody binding terminus can bind to a Fab region of an antibody.

In any aspect or embodiment described herein, an antibody-recruiting molecule (ARM) comprises:
an antibody binding moiety (ABM) or an antibody binding terminus (ABT),
a target binding moiety (TBM) or target binding terminus (TBT), and
optionally a linker moiety that covalently links the ABM/ABT to the TBM/TBT, wherein the antibody binding moiety or antibody binding terminus can bind to a Fc region of an antibody.

In any aspect or embodiment described herein, an antibody-recruiting molecule (ARM) comprises:
an antibody binding moiety (ABM) or an antibody binding terminus (ABT),
a target binding moiety (TBM) or target binding terminus (TBT), and
optionally a linker moiety that covalently links the ABM/ABT to the TBM/TBT,
wherein the antibody binding moiety or antibody binding terminus can bind to two or more antibodies which have different Fab regions.

In any aspect or embodiment described herein, ARMs can significantly improve efficiency of NK cell therapy, for example, by facilitating the recognition of target cells by NK cells. In any aspect or embodiment described herein, ARMs can enhance interactions between NK cells and their target cells, and/or promote target cell deaths induced by NK cells. In any aspect or embodiment described herein, ARMs promote activation of NK cells, for example, by recruiting antibodies to target cells, which antibodies can then recruit and activate NK cells.

Additionally or alternatively, in any aspect or embodiment described herein, ARMs reduce and/or delay onset of one or more side effects of NK cell therapy, for example, by directing NK cells to target cells and reducing off-target effects. In any aspect or embodiment described herein, NK cells are typically activated, enriched and/or expanded before administration to a subject. Such pre-activated NK cells are more powerful and may be necessary for efficient killing of target cells, however, due to their activation, these cells may cause significantly more side effects, e.g., killing of unintended cells. Among other things, the present disclosure provides technologies by which ARMs can direct NK cells to target cells and reduce side effects and toxicity associated with NK cell therapy. Further, in view of reduced off-target effects, in any aspect or embodiment described herein, more activated NK cells may be utilized to further improve therapeutic efficiency.

Among other things, an antibody binding moiety (also referred to herein as an antibody binding terminus (ABT)) can bind to antibodies (or fragments thereof). In any aspect or embodiment described herein, antibody binding moieties bond to variable regions. In any aspect or embodiment described herein, antibody binding moieties bind to Fab regions. In any aspect or embodiment described herein, antibody binding moieties can, among other things, recruit antibodies (or fragments thereof) of various antigen-specificity, e.g., through binding to Fc regions (“universal ABTs”, or “uABTs”). In any aspect or embodiment described herein, utilization of uABTs can circumvent dependence of specific antibody populations and undesirable effects that may result from individual variations of specific antibody populations. In any aspect or embodiment described herein, uABTs enables recruitment preferentially of IgG1, IgG2, and/or IgG4. In any aspect or embodiment described herein, recruitment of antibodies, e.g., IgG subclasses, is limited by the administered dose of an ARM, and/or is not by levels of antibodies having a particular Fab region in an individual. In any aspect or embodiment described herein, ABTs bind to and recruit endogenous antibodies. In any aspect or embodiment described herein, uABTs bind to IgG molecules, preferably the uABTs bind to IgG (such as human IgG) over human IgA or IgM. Typically, after being bound by antibody binding moieties, antibodies remain capable of performing one or more of their immune activities, e.g., interactions with NK cells which can lead to killing of target cells by NK cells.

In any aspect or embodiment described here, an antibody binding moiety, e.g., a universal antibody binding moiety, binds to an Fc region of an antibody. In any aspect or embodiment described here, an antibody binding moiety, e.g., a universal antibody binding moiety, binds to a conserved Fc region of an antibody. In any aspect or embodiment described here, an antibody binding moiety binds to an Fc region of an IgG antibody.

Those skilled in the art will appreciate that the technologies described herein are suitable for various systems, subjects, or patients, such as those described herein. For example, in any aspect or embodiment described herein, a system, subject, or patient of the present disclosure comprises or is an organism. In any aspect or embodiment described herein, a system of the present disclosure comprises or is a subject. In any aspect or embodiment described herein, a system, subject, or patient of the present disclosure comprises or is a human. In any aspect or embodiment described herein, a system of the present disclosure comprises or is a patient. In any aspect or embodiment described herein, a system, subject, or patient of the present disclosure comprises or is a non-human animal. In any aspect or embodiment described herein, a system, subject, or patient comprises or is a non-human primate, such as a non-human primate useful as a disease model. In any aspect or embodiment described herein, a system, subject, or patient comprises or is a non-human animal, such as a non-human animal useful as a disease model. In any aspect or embodiment described herein, a system of the present disclosure comprises or is an organ. In any aspect or embodiment described herein, a system of the present disclosure comprises or is a tissue. In any aspect or embodiment described herein, a system of the present disclosure comprises or is an in vivo system. In any aspect or embodiment described herein, a system of the present disclosure comprises or is an ex vivo system. In any aspect or embodiment described herein, a system of the present disclosure comprises or is an in vitro system. In any aspect or embodiment described herein, a system of the present disclosure is or comprises cell culture.

In any aspect or embodiment described herein, the number of coronavirus infected cells in the system or the subject is reduced compared to absence of both the natural killer cells and the compound of the present disclosure. In any aspect or embodiment described herein, growth of coronavirus infected cells in the system or the subject is reduced compared to absence of both the natural killer cells and the antibody-recruiting molecule. In any aspect or embodiment described herein, proliferation of coronavirus infected cell in the system or the subject is reduced compared to absence of both the natural killer cells and the antibody-recruiting molecule.

In any aspect or embodiment described herein, compounds or ARMs of the present disclosure can be administered prior to, concurrent with, and/or subsequent to NK cells (e.g., expanded, enriched, and/or pre-activated NK cells).

### DETAILED DESCRIPTION

Various ARM agents or compounds may be utilized in accordance with the present disclosure. In any aspect or embodiment described herein, ARMs comprise antibody binding moieties that interact with Fab regions, and may preferably bind to and recruit antibodies (and/or fragments thereof) that contain such Fab regions. In any aspect or embodiment described herein, ARMs comprise antibody binding moieties that interact with Fc regions (e.g., uABTs described herein), and may bind to and recruit various antibodies (and/or fragments thereof) that contain such Fc regions. As appreciated by those skilled in the art, antibodies (and/or fragments thereof) recruited by such ARMs may contain diverse Fab regions and may have diverse specificity. In any aspect or embodiment described herein, no individual recruited antibodies (or fragments thereof) are more than 1%, 2%, 3%, 4%, 5%, 6%, 7%, 8%, 9%, 10%, 15%, 20%, 25%, 30%, 35%, 40%, 45%, 50%, 60%, 70%, 80%, or 90% of total recruited antibodies and fragments thereof.

In any aspect or embodiment described herein, a compound or ARM, or a pharmaceutically acceptable salt thereof, comprises:
one or more (e.g., 1, 2, 3, 4, 5, 6, 7, 8, 9, 10, or more) antibody binding moiety (ABM or an antibody binding terminus (ABT)) that binds to one or more antibodies or fragments thereof,
one or more (e.g., 1, 2, 3, 4, 5, 6, 7, 8, 9, 10, or more) target binding moiety (TBM or target binding terminus (TBT)) that binds the spike glycoprotein of a coronavirus (e.g., severe acute respiratory syndrome coronavirus 2 (SARS-CoV-2)), and
optionally a linker moiety (L) that covalently links the one or more ABMs and the one or more TBMs.

In any aspect or embodiment described herein, a compound or ARM, or a pharmaceutically acceptable salt thereof comprises the chemical structure:

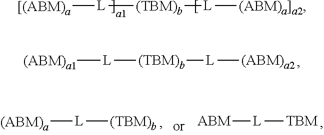

wherein
L is a bond or a linker moiety that covalently links the one or more ABMs and the one or more TBMs;
a is 1, 2, 3, 4, 5, 6, 7, 8, 9, or 10 (e.g., 1, 2, or 3, preferably 1);
a1 is 1, 2, 3, 4, or 5 (e.g., 1 or 2, preferably 1);
a2 is 1, 2, 3, 4, or 5 (e.g., 1 or 2, preferably 1); and
b is 1, 2, 3, 4, 5, 6, 7, 8, 9, or 10 (e.g., 1, 2, or 3, preferably 1).

In any aspect or embodiment described herein, a compound or ARM, or a pharmaceutically acceptable salt thereof, comprises the chemical structure:

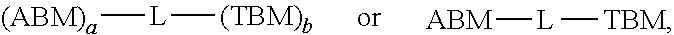

wherein:
   a is 1, 2, or 3 (preferably 1); and
   b is 1, 2, or 3 (preferably 1).

In any aspect or embodiment described herein, the present disclosure utilizes compounds or ARM agents of the present disclosure together with cellular immunotherapy, such as NK cell therapy (e.g., involving in vitro expansion of NK cells). In any aspect or embodiment described herein, one or more compounds or ARM agents as described herein is administered to a subject who has received, is receiving or will receive NK cell therapy, or otherwise has or will have a population of NK cells targeted to a site of interest (e.g., to a tumor). Conversely, in any aspect or embodiment described herein, cellular immunotherapy (e.g. NK cell therapy) is administered to a subject who has received, is receiving, or will receive therapy with a compound or ARM agent as described herein.

In any aspect or embodiment described herein, the present disclosure utilizes compounds or ARM agents that comprise antibody binding moieties that can bind to antibodies (or fragments thereof) with different Fab structures (“uABT”). Particularly, in any aspect or embodiment described herein, relevant ARM agents comprise antibody binding moieties that bind to Fc region of antibodies. In any aspect or embodiment described herein, such binding to Fc regions of antibodies does not interfere one or more immune activities of the antibodies, e.g., interaction with Fc receptors (e.g., CD16a), recruitment of effector cells like NK cells for ADCC, etc. As those skilled in the art will appreciate, provided technologies (agents, compounds, compositions, methods, etc.) of the present disclosure comprising ABMs, such as uABTs, can provide various advantages—for example, the compounds or ARM agents can utilize antibodies having various Fab regions in the immune system to avoid or minimize undesired effects of antibody variations among a patient population, and can trigger and/or enhance immune activities toward targets, e.g., killing target diseased cells such as cancer cells.

In any aspect or embodiment described herein, compounds or ARM agents of the present disclosure are useful for recruiting antibodies and NK cells to various targets, e.g., cancer cells. In any aspect or embodiment described herein, compounds or ARM agents of the present disclosure are useful for modulating immune activities, such as ADCC involving NK cells, against targets (diseased cells, foreign objects or entities, etc.). In any aspect or embodiment described herein, compounds or ARM agents of the present disclosure are useful for modulating NK cell activities against target cells, e.g., diseased cells such as cancer cells.

In any aspect or embodiment described herein, a compound or agent (e.g., an ARM) comprises:
an antibody binding moiety,
a target binding moiety that binds the spike glycoprotein of a coronavirus (e.g., severe acute respiratory syndrome coronavirus 2 (SARS-CoV-2)), and
optionally a linker moiety,
wherein the antibody binding moiety can bind to a Fab region.

In any aspect or embodiment described herein, a compound or agent (e.g., an ARM) comprises:
an antibody binding moiety,
a target binding moiety that binds the spike glycoprotein of a coronavirus (e.g., severe acute respiratory syndrome coronavirus 2 (SARS-CoV-2)), and
optionally a linker moiety,
wherein the antibody binding moiety can bind to a Fc region.

In any aspect or embodiment described herein, a compound or agent (e.g., an ARM) comprises:
an antibody binding moiety,
a target binding moiety that binds the spike glycoprotein of a coronavirus (e.g., severe acute respiratory syndrome coronavirus 2 (SARS-CoV-2)), and
optionally a linker moiety,
wherein the antibody binding moiety can bind to two or more antibodies which have different Fab regions.

In any aspect or embodiment described herein, compounds or agents of the present disclosure comprise two or more antibody binding moieties. In any aspect or embodiment described herein, useful compounds or agents of the present disclosure comprise two or more target binding moieties.

An antibody binding moiety may interact with any portion of an antibody. In any aspect or embodiment described herein, an antibody binding moiety binds to an Fc region of an antibody. In any aspect or embodiment described herein, an antibody binding moiety binds to a conserved Fc region of an antibody. In any aspect or embodiment described herein, an antibody binding moiety binds to an Fc region of an IgG antibody. As appreciated by those skilled in the art, various antibody binding moieties, linkers, and target binding moieties can be utilized in accordance with the present disclosure. Among other things, as demonstrated in the Examples, in any aspect or embodiment described herein, the present disclosure provides antibody binding moieties, linkers, and target binding moieties and combinations thereof that are particularly useful and effective for constructing ARM molecules to recruit antibodies to target cells, and/or to trigger, generate, encourage, and/or enhance immune system activities toward target cells (e.g., cells infected with a coronavirus, such as SARS-CoV-2).

In any aspect or embodiment described herein, antibody binding moieties can bind to a Fc region that is bound to Fc receptors, e.g., FcγRIIIa, CD16a, etc. In any aspect or embodiment described herein, provided moieties and/or agents (e.g., compounds/ARMs of various formulae as described in the present disclosure) comprise antibody binding moieties that bind to a complex comprising an Fc region and an Fc receptor. In any aspect or embodiment described herein, a complex comprises:
an agent comprising:
   an antibody binding moiety,
   a target binding moiety that binds the spike glycoprotein of a coronavirus (e.g., severe acute respiratory syndrome coronavirus 2 (SARS-CoV-2)), and
   optionally a linker moiety,
an Fc region, and
an Fc receptor,
wherein the antibody binding moiety of the agent can bind to two or more antibodies which have different Fab regions.

In any aspect or embodiment described herein, an Fc region is an Fc region of an endogenous antibody of a subject. In any aspect or embodiment described herein, an Fc region is an Fc region of an exogenous antibody. In any aspect or embodiment described herein, an Fc region is an Fc region of an administered agent. In any aspect or embodiment described herein, an Fc receptor is of a diseased cell in a subject. In any aspect or embodiment described herein, an Fc receptor is of a coronavirus infected cell (such as, a SARS-CoV-2 infected cell) in a subject.

In any aspect or embodiment described herein, ABT is a universal antibody binding moiety.

In any aspect or embodiment described herein, an antibody binding moiety comprises one or more amino acid residues. In any aspect or embodiment described herein, an antibody binding moiety is or comprises a peptide moiety. In any aspect or embodiment described herein, an antibody binding moiety is or comprises a cyclic peptide moiety. In any aspect or embodiment described herein, such antibody binding moiety comprises one or more natural (i.e., proteinogenic) amino acid residues. In any aspect or embodiment described herein, such antibody binding moiety comprises one or more unnatural natural (i.e., nonproteinogenic) amino acid residues.

In any aspect or embodiment described herein, a is 1. In any aspect or embodiment described herein, b is 1. In any aspect or embodiment described herein, a is 1 and b is 1. In any aspect or embodiment described herein, a1 is 1. In any aspect or embodiment described herein, a2 is 1. In any aspect or embodiment described herein, a1 is 1 and a2 is 1.

In any aspect or embodiment described herein, two or more side chains of the amino acid residues (e.g., R^{a2} or R^{a3} of one amino acid residue with R^{a2} or R^{a3} of another amino acid residue) are optionally take together to form a bridge (e.g., in any aspect or embodiment described herein, two cysteine residues form a —S—S— bridge as typically observed in natural proteins). In any aspect or embodiment described herein, a formed bridge has the structure of L^{b}, as described herein. In any aspect or embodiment described herein, each end of L^{b} independently connects to a backbone atom of a cyclic peptide. In any aspect or embodiment described herein, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In any aspect or embodiment described herein, L^{b} connects to a ring through a side chain of an amino acid residue. In any aspect or embodiment described herein, such a side chain comprises an amino group or a carboxylic acid group.

In any aspect or embodiment described herein,

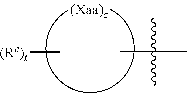

is an antibody binding moiety (

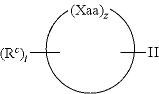

binds to an antibody). In any aspect or embodiment described herein,

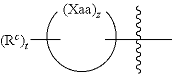

is a universal antibody binding moiety. In any aspect or embodiment described herein,

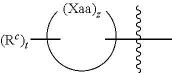

is a universal antibody binding moiety which can bind to antibodies having different Fab regions. In any aspect or embodiment described herein,

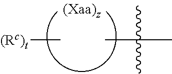

is a universal antibody binding moiety that can bind to a Fc region. In any aspect or embodiment described herein, an antibody binding moiety, e.g., a universal antibody binding moiety having the structure of

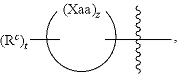

can bind to a Fc region bound to an Fc receptor. In any aspect or embodiment described herein, an antibody binding moiety, e.g., of an antibody binding moiety having the structure of

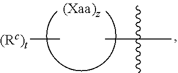

has the structure of

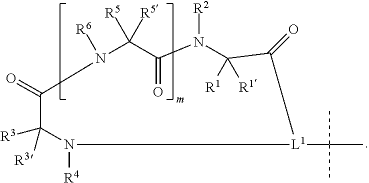

In any aspect or embodiment

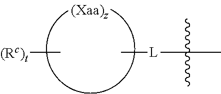

described herein, has the structure of

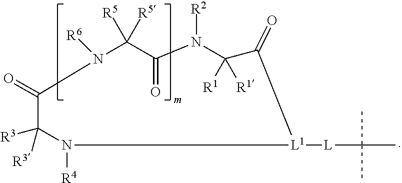

In any aspect or embodiment described herein, a compound or agent (e.g., an ARM) has the chemical structure:

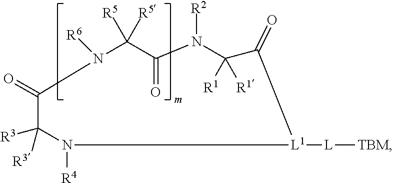

or a pharmaceutically acceptable salt thereof, wherein the variables are independently defined as described in any aspect or embodiment described herein.

In any aspect or embodiment describe herein, a1 is 1. In any aspect or embodiment described herein, a2 is 1. In any aspect or embodiment described herein, b is 1. In any aspect or embodiment described herein, a compound or agent has the structure

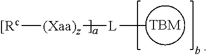

In any aspect or embodiment described herein, a compound or agent has the structure

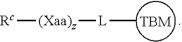

In any aspect or embodiment described herein, a compound or agent has the structure

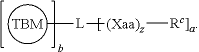

In any aspect or embodiment described herein, a compound or agent has the structure

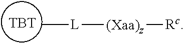

In any aspect or embodiment described herein, each Xaa independently has the structure of —N(R^{a1}-L^{a1}-C(R^{a2})(R^{a3})-L^{a2}-CO—, wherein R^{a1}, L^{a1}, R^{a2}, R^{a3}, and L^{a2} are each independently defined as described in any aspect or embodiment described herein. In any aspect or embodiment described herein, two or more side chains of the amino acid residues (e.g., in compounds R^{a2} or R^{a3} of one amino acid residue with R^{a2} or R^{a3} of another amino acid residue) are optionally take together to form a bridge (e.g., in any aspect or embodiment described herein, two cysteine residues form a —S—S— bridge as typically observed in natural proteins). In any aspect or embodiment described herein, a formed bridge has the structure of L^{b}, as described herein. In any aspect or embodiment described herein, each end of L^{b} independently connects to a backbone atom of a cyclic peptide (e.g., a ring atom of the ring formed by —(Xaa)_{z}-). In any aspect or embodiment described herein, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In any aspect or embodiment described herein, L^{b} connects to a ring through a side chain of an amino acid residue. In any aspect or embodiment described herein, such a side chain comprises an amino group or a carboxylic acid group.

In any aspect or embodiment described herein, R^{c}-(Xaa)z- is an antibody binding moiety (R^{c}-(Xaa)z-H binds to an antibody). In any aspect or embodiment described herein, R^{c}-(Xaa)z- is a universal antibody binding moiety. In any aspect or embodiment described herein, R^{c}-(Xaa)z- is a universal antibody binding moiety which can bind to antibodies having different Fab regions. In any aspect or embodiment described herein, R^{c}-(Xaa)z- is a universal antibody binding moiety that can bind to a Fc region. In any aspect or embodiment described herein, an antibody binding moiety, e.g., a universal antibody binding moiety having the structure of R-(Xaa)z-, can bind to a Fc region which binds to an Fc receptor. In any aspect or embodiment described herein, R^{c}-(Xaa)z- has the structure of

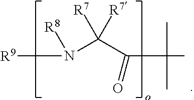

In any aspect or embodiment described herein, R^{c}-(Xaa)z-L- has the structure of

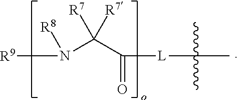

In any aspect or embodiment described herein, a compound or agent (e.g., an ARM), has the chemical structure:

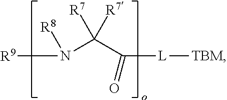

or a pharmaceutically acceptable salt thereof, wherein the variables are independently defined as described in any aspect or embodiment described herein 0.

### Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is an ARM as described herein.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation, or combinations thereof. Unless otherwise specified, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof.

Alkenyl: As used herein, the term “alkenyl” refers to an aliphatic group, as defined herein, having one or more double bonds.

Alkenylene: The term “alkenylene” refers to a bivalent alkenyl group.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkylene: The term “alkylene” refers to a bivalent alkyl group.

Amino acid: In its broadest sense, as used herein, refers to any compound and/or substance that can be incorporated into a polypeptide chain, e.g., through formation of one or more peptide bonds. In some embodiments, an amino acid comprising an amino group and a carboxylic acid group. In some embodiments, an amino acid has the general structure NH(R′)—C(R′)₂—COOH, wherein each R′ is independently as described in the present disclosure. In some embodiments, an amino acid has the general structure H₂N—C(R′)₂—COOH, wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi- specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; KALBITOR®s; CovX-Bodies; and CrossMabs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, cells, etc.) is considered to be associated with a particular disease, disorder, or condition, if its presence, level and/or form correlates with incidence of and/or susceptibility to the disease, disorder, or condition (e.g., across a relevant population).

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Carbocyclyl: The term “carbocyclyl” is a monocyclic or bicyclic saturated, partially unsaturated, or aromatic ring system in which all ring atoms are carbon. Usually, each ring of the carbocyclyl group contains from 3-6 ring atoms and a bicyclic carbocyclyl group contains from 7 to 10 ring atoms, but some other number of ring atoms may be specified. Unless otherwise indicated, the carbocycle may be attached to the group it substitutes at any carbon atom that results in a stable structure. When indicated the carbocyclic rings described herein may be substituted at any carbon atom if the resulting compound is stable. Examples of carbocyclyl groups include phenyl, naphthyl, tetrahydronaphthyl, cyclopropyl, cyclohexyl, and cyclohexenyl.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 14 ring atoms (i.e., monocyclic, bicyclic or polycyclic), in some embodiments 5, 6, 9, 10 or 14 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” or “heteroaryl group,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 3- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Pharmaceutical composition: As used herein, the term “pharmaceutical composition” refers to an active agent, e.g., a compound, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds with bases. Representative alkali or alkaline earth metal salts include salts of sodium, lithium, potassium, calcium, magnesium, and the like. In some embodiments, pharmaceutically acceptable salts are ammonium salts (e.g., —N(R)₃+). In some embodiments, pharmaceutically acceptable salts are sodium salts. In some embodiments, pharmaceutically acceptable salts are calcium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound is in the form of a pharmaceutically acceptable salt.

Protecting Group: The phrase “protecting group” or “protection group,” as used herein, refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, propionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxycarbonylamino (—NHCBZ), allylamino, benzylamino (—NHBn), fluorenylmethoxycarbonylamino (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)N(R^{∘})₂; —N(R^{∘})C(S)N(R^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)N(R^{∘})₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSi(R^{∘})₃; —(CH₂)₀₋₄OC(O)R^{∘}; —OC(O)(CH₂)₀₋₄SR^{∘}, —(CH₂)₀₋₄SC(O)R^{∘}; —(CH₂)₀₋₄C(O)N(R^{∘})₂; —C(S)N(R^{∘})₂; —C(S)SR^{∘}; —SC(S)SR^{∘}, —(CH₂)₀₋₄OC(O)N(R^{∘})₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂N(R^{∘})₂; —(CH₂)₀₋₄S(O)R^{∘}; —N(R^{∘})S(O)₂N(R^{∘})₂; —N(R^{∘})S(O)₂R^{∘}; —N(OR^{∘})R^{∘}; —C(NH)N(R^{∘})₂; —Si(R^{∘})₃; —OSi(R^{∘})₃; —OSi(OR^{∘})₃; —P(R^{∘})₂; —P(OR^{∘})₂; —OP(R^{∘})₂; —OP(OR^{∘})₂; —N(R^{∘})P(R^{∘})₂; —OP(O)(R^{∘})₂; —OP(O)(OR^{∘})₂; —N(R^{∘})P(O)(R^{∘})₂; —N(R^{∘})P(O)(OR^{∘})₂; —B(R^{∘})₂; —OB(R^{∘})₂; —OB(OR^{∘})₂; —P(O)(R^{∘})₂; —OP(O)(R^{∘})₂;—(C₁-4 straight or branched alkylene)O—N(R^{∘})₂; or —(C₁₋₄ straight or branched alkylene)C(O)O—N(R^{∘})₂; wherein each R^{∘} may be substituted as defined below and is independently hydrogen, C₁₋₂₀ aliphatic, C₁-20 heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{c}, taken together with their intervening atom(s), form a 3-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR′, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{●} include ═O and ═S.

Suitable divalent substituents, e.g., on a suitable carbon atom, nitrogen atom, etc., include independently the following: ═O, ═S, ═CR*₂, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each R* may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₂₀ aryl), —O(CH₂)₀₋₁(C₆₋₂₀ aryl), —CH₂-(5-20 membered heteroaryl ring having 1-5 heteroatoms independently selected from nitrogen, oxygen, sulfur, silicon and phosphorus), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R, taken together with their intervening atom(s), form a 3-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below. Suitable divalent substituents that are bound to vicinal substitutable atoms of an “optionally substituted” group include:—O(CR*₂)₂₋₃O—.

Suitable monovalent substituents on R* (or the ring formed by taking two independent occurrences of R* together with their intervening atoms), are independently halogen, —(CH₂)₀₋₂R^{●}, -(haloR^{●}), —(CH₂)₀₋₂OH, —(CH₂)₀₋₂OR′, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents of R* include ═O and ═S.

In some embodiments, suitable substituents on a substitutable nitrogen of an “optionally substituted” group include—R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted—OPh, or an unsubstituted 5-6 membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

In some embodiments, suitable substituents on the aliphatic group of R^{\} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R′ is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, provided compounds are useful as therapeutic agents.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount. In some embodiments, provided compounds are administered in therapeutically effective amount, either in a single dose or in multiple unit doses.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Unless otherwise specified, salts, such as pharmaceutically acceptable acid or base addition salts, stereoisomeric forms, and tautomeric forms, of provided compounds (e.g., agents, ARMs, etc.) are included. Unless otherwise specified, structures depicted herein also include compounds that comprise various isotopes, e.g., replacement of hydrogen by deuterium or tritium, replacement of a carbon by a ¹³C or ¹⁴C, etc. In some embodiments, a composition comprising a compound comprises an enriched level of an isotope in the compound. Such compounds and compositions are useful, among other things, as analytical tools, as probes in biological assays, or as therapeutic agents, etc.

Unless otherwise clear from context, in the present disclosure, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

### Exemplified Arm Agents

Various ARM agents may be utilized in accordance with the present disclosure. In some embodiments, ARMs comprise antibody binding moieties that interact with Fab regions, and may preferably bind to and recruit antibodies (and/or fragments thereof) that contain such Fab regions. In some embodiments, ARMs comprise antibody binding moieties that interact with Fc regions (e.g., uABTs described herein), and may bind to and recruit various antibodies (and/or fragments thereof) that contain such Fc regions. As appreciated by those skilled in the art, antibodies (and/or fragments thereof) recruited by such ARMs may contain diverse Fab regions and may have diverse specificity. In some embodiments, no individual recruited antibodies (or fragments thereof) are more than 1%, 2%, 3%, 4%, 5%, 6%, 7%, 8%, 9%, 10%, 15%, 20%, 25%, 30%, 35%, 40%, 45%, 50%, 60%, 70%, 80%, or 90% of total recruited antibodies and fragments thereof.

In any aspect or embodiment described herein, a compound or ARM, or a pharmaceutically acceptable salt thereof, comprises:
one or more (e.g., 1, 2, 3, 4, 5, 6, 7, 8, 9, 10, or more) antibody binding moiety (ABM or an antibody binding terminus (ABT)) that binds to one or more antibodies or fragments thereof,
one or more (e.g., 1, 2, 3, 4, 5, 6, 7, 8, 9, 10, or more) target binding moiety (TBM or target binding terminus (TBT)) that binds the spike glycoprotein of a coronavirus (e.g., severe acute respiratory syndrome coronavirus 2 (SARS-CoV-2)), and
optionally a linker moiety (L) that covalently links the one or more ABMs and the one or more TBMs.

In any aspect or embodiment described herein, a compound or ARM, or a pharmaceutically acceptable salt thereof comprises the chemical structure:

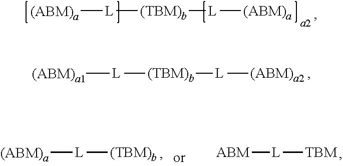

wherein
   L is a bond or a linker moiety that covalently links the one or more ABMs and the one or more TBMs;
   a is 1, 2, 3, 4, 5, 6, 7, 8, 9, or 10 (e.g., 1, 2, or 3, preferably 1);
   a1 is 1, 2, 3, 4, or 5 (e.g., 1 or 2, preferably 1);
   a2 is 1, 2, 3, 4, or 5 (e.g., 1 or 2, preferably 1); and
   b is 1, 2, 3, 4, 5, 6, 7, 8, 9, or 10 (e.g., 1, 2, or 3, preferably 1).

In any aspect or embodiment described herein, a compound or ARM, or a pharmaceutically acceptable salt thereof, comprises the chemical structure:

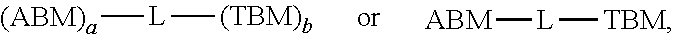

wherein:
   a is 1, 2, or 3 (preferably 1); and
   b is 1, 2, or 3 (preferably 1).

In any aspect or embodiment described herein, a compound or ARM, or a pharmaceutically acceptable salt thereof, the N-terminus of the ABM, the TBM, or both are acetylated (—COCH₃).

In any aspect or embodiment described herein, a compound or ARM, or a pharmaceutically acceptable salt thereof, the C-terminus of the ABM, the TBM, or both are amidated (—NH₂).

### Exemplified Antibody Binding Moieties (ABM)

Various antibody binding moieties are known in the art and may be utilized in accordance with the present disclosure.

In any aspect or embodiment described herein, antibody binding moieties primarily interact with Fab regions. In any aspect or embodiment described herein, antibody binding moieties primarily interact with antibodies and/or fragments thereof that comprise certain Fab structures and/or are directed to certain antigens.

In any aspect or embodiment described herein, antibody binding moieties interact with Fc regions. In any aspect or embodiment described herein, antibody binding moieties described herein (e.g. uABTs) interact with Fc regions and may recruit Fc regions connected to Fab regions having diverse structures and/or antigen specificity. In any aspect or embodiment described herein, compounds of the present disclosure comprise one or more universal antibody binding moiety, each that can bind to antibodies having different Fab regions and different specificity. In any aspect or embodiment described herein, antibody binding moieties are universal antibody binding moieties that bind to Fc regions. In any aspect or embodiment described herein, binding of antibody binding moieties to antibodies or fragments thereof (e.g. by uABTs to Fc regions) can happen at the same time as binding of Fc receptors, e.g., CD16a, to the same antibodies or fragments thereof (e.g., the Fc receptors and antibody binding moiety may bind/interact at different locations/amino acid residues of the same Fc regions in case of uABT binding to Fc). In any aspect or embodiment described herein, upon binding of universal antibody binding moieties, an Fc region can still interact with Fc receptors and perform one or more or all of its immune activities, including recruitment of immune cells (e.g. effector cells, such as NK cells), and/or triggering, generating, encouraging, and/or enhancing immune system activities toward target cells, tissues, objects and/or entities (e.g., antibody-dependent cell-mediated cytotoxicity (ADCC) and/or antibody-dependent cellular phagocytosis (ADCP)).

Various universal antibody binding moieties can be utilized in accordance with any aspect or embodiment of the present disclosure, and many technologies, such as those described in the Examples described herein, are available for identifying and/or assessing universal antibody binding moieties. In any aspect or embodiment described herein, a universal antibody binding moiety comprises one or more amino acid residues, each independently a proteinogenic (natural) amino acid or a non-proteinogenic (unnatural) amino acid. For example, non-proteinogenic (unnatural) amino acid includes, e.g., 1,2,3,4-tetrahydroisoquinoline-3-carboxylic acid or 2-aminoisobutyric acid.

In any aspect or embodiment described herein, a universal antibody binding moiety is or comprises a peptide moiety (e.g., a moiety having the structure of R^{c}-(Xaa)z-). In any aspect or embodiment described herein, a universal antibody binding moiety is or comprises a cyclic peptide moiety (e.g., a moiety having the structure of

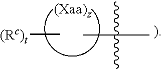

In any aspect or embodiment described herein, a universal antibody binding moiety is R^{c}-(Xaa)z- or

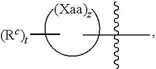

and is or comprises a peptide unit. In any aspect or embodiment described herein, -(Xaa)z- is or comprises an ABM peptide unit. In any aspect or embodiment described herein, an ABM peptide unit comprises an amino acid residue (e.g., a residue of an amino acid of formula NH(R^{a1})-L^{a1}-C(R^{a2})(R^{a3})-L^{a2}-COOH) that has a positively charged side chain (e.g., at physiological pH of about 7.4, “positively charged amino acid residue”, Xaa^{A}). In any aspect or embodiment described herein, an ABM peptide unit comprises R. In any aspect or embodiment described herein, at least one Xaa is R. In any aspect or embodiment described herein, an ABM peptide unit is or comprises APAR. In any aspect or embodiment described herein, an ABM peptide unit is or comprises RAPA. In any aspect or embodiment described herein, an ABM peptide unit comprises an amino acid residue (e.g., a residue of an amino acid of formula NH(R^{a1})-L^{a1}-C(R^{a2})(R^{a3})-L^{a2}-COOH) that has a side chain comprising an aromatic group (“aromatic amino acid residue”, Xaa^{A}). In any aspect or embodiment described herein, an ABM peptide unit comprises a positively charged amino acid residue and an aromatic amino acid residue. In any aspect or embodiment described herein, an ABM peptide unit comprises W. In any aspect or embodiment described herein, an ABM peptide unit comprises a positively charged amino acid residue and an aromatic amino acid residue. In any aspect or embodiment described herein, an ABM peptide unit is or comprises Xaa^{A}XaaXaa^{P}Xaa^{p}. In any aspect or embodiment described herein, an ABM peptide unit is or comprises Xaa^{P}Xaa^{P}XaaXaa^{A}. In any aspect or embodiment described herein, an ABM peptide unit is or comprises Xaa^{P}Xaa^{A}Xaa^{P}. In any aspect or embodiment described herein, an ABM peptide unit is or comprises two or more Xaa^{P}Xaa^{A}Xaa^{P}. In any aspect or embodiment described herein, an ABM peptide unit is or comprises Xaa^{P}Xaa^{A}Xaa^{P}XaaXaa^{P}Xaa^{A}Xaa^{P}. In any aspect or embodiment described herein, an ABM peptide unit is or comprises Xaa^{P}Xaa^{P}Xaa^{A}Xaa^{A}Xaa^{P}. In any aspect or embodiment described herein, an ABM peptide unit is or comprises Xaa^{P}Xaa^{P}Xaa^{P}Xaa^{A}. In any aspect or embodiment described herein, an ABM peptide unit is or comprises two or more Xaa^{A}Xaa^{A}Xaa^{P}. In any aspect or embodiment described herein, an ABM peptide residue comprises one or more proline residues. In any aspect or embodiment described herein, an ABM peptide unit is or comprises the amino acid sequence HWRGWA. In any aspect or embodiment described herein, an ABM peptide unit is or comprises the amino acid sequence WGRR. In any aspect or embodiment described herein, an ABM peptide unit is or comprises the amino acid sequence RRGW. In any aspect or embodiment described herein, an ABM peptide unit is or comprises the amino acid sequence NRFRGKYK. In any aspect or embodiment described herein, an ABM peptide unit is or comprises the amino acid sequence NARKFYK. In any aspect or embodiment described herein, an ABM peptide unit comprises a positively charged amino acid residue, an aromatic amino acid residue, and an amino acid residue (e.g., a residue of an amino acid of formula NH(R^{a1})-L^{a1}-C(R^{a2})(R^{a3})-L^{a2}-COOH) that has a negatively charged side chain (e.g., at physiological pH about 7.4, “negatively charged amino acid residue”, Xaa^{N}). In any aspect or embodiment described herein, an ABM peptide residue is or comprises the amino acid sequence RHRFNKD. In any aspect or embodiment described herein, an ABM peptide unit is or comprises the amino acid sequence TY. In any aspect or embodiment described herein, an ABM peptide unit is or comprises the amino acid sequence TYK. In any aspect or embodiment described herein, an ABM peptide unit is or comprises the amino acid sequence RTY. In any aspect or embodiment described herein, an ABM peptide unit is or comprises the amino acid sequence RTYK. In any aspect or embodiment described herein, an ABM peptide unit is or comprises the amino acid sequence of PAM. In any aspect or embodiment described herein, an ABM peptide unit is or comprises the amino acid sequence WHL. In any aspect or embodiment described herein, an ABM peptide unit is or comprises the amino acid sequence ELVW. In any aspect or embodiment described herein, an ABM peptide unit is or comprises the amino acid sequence AWHLGELVW. In any aspect or embodiment described herein, an ABM peptide unit is or comprises the amino acid sequence DCAWHLGELVWCT, which the two cysteine residues can form a disulfide bond as found in natural proteins. In any aspect or embodiment described herein, an ABM peptide unit is or comprises the amino acid sequence of Fc-III. In any aspect or embodiment described herein, an ABM peptide unit is or comprises the amino acid sequence DpLpAWHLGELVW. In any aspect or embodiment described herein, an ABM peptide unit is or comprises the amino acid sequence of FcBP-1. In any aspect or embodiment described herein, an ABM peptide unit is or comprises the amino acid sequence DpLpDCAWHLGELVWCT. In any aspect or embodiment described herein, an ABM peptide unit is or comprises the amino acid sequence of FcBP-2. In any aspect or embodiment described herein, an ABM peptide unit is or comprises the amino acid sequence CDCAWHLGELVWCTC, wherein the first and the last cysteines, and the two cysteines in the middle of the sequence, can each independently form a disulfide bond as in natural proteins. In any aspect or embodiment described herein, an ABM peptide unit is or comprises the amino acid sequence of Fc-III-4c. In any aspect or embodiment described herein, a peptide unit is or comprises the amino acid sequence of FcRM. In any aspect or embodiment described herein, an ABM peptide unit is or comprises a cyclic ABM peptide unit. In any aspect or embodiment described herein, a cyclic ABM peptide unit comprises an amide group formed by an amino group of a side chain and the C-terminus—COOH.

In any aspect or embodiment described herein, one or more (such as each) ABM or universal antibody binding moiety is independently selected from:

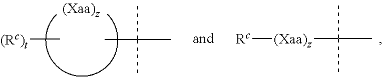

wherein:
each Xaa is an independently selected amino acid residue (such as, a natural/proteinogenic amino acid, an unnatural/non-proteinogenic amino acid, or a combination thereof);
t is an integer from 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, or 50);
z is an integer from 3 to 50 (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, or 50);
each R^{c} is independently -L^{a}-R′;
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₁₋₃₀ arylaliphatic, C₁₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In any aspect or embodiment described herein, a universal antibody binding moiety has the structure of

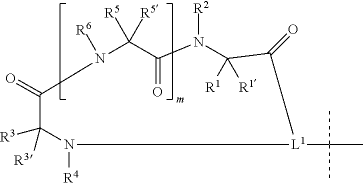

or salt form thereof, wherein:
each R¹, R³ and R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms, a 5-6 membered monocyclic heteroaromatic ring having 1-4 heteroatoms, or an 8-10 membered bicyclic heteroaromatic ring having 1-5 heteroatoms; or:
R¹ and R^{1′} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms;
R³ and R^{3′} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms;
R⁵ and R^{5′} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms; or
two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)-, —N(R)C(O)-, —S(O)—, —S(O)₂—, or -Cy¹-, wherein each -Cy¹- is independently a 5-6 membered heteroarylenyl with 1-4 heteroatoms;
each R^{1′}, R^{3′} and R^{5′} is independently hydrogen or optionally substituted C₁₋₃ aliphatic;
each R², R⁴ and R⁶ is independently hydrogen, or optionally substituted C₁₋₄ aliphatic, or:
   R² and R¹ are optionally taken together with their intervening atoms to form a 4-11 membered, optionally substituted saturated or partially unsaturated monocyclic heterocyclic ring having 1-2 heteroatoms;
   R⁴ and R³ are optionally taken together with their intervening atoms to form a 4-11 membered optionally substituted saturated or partially unsaturated monocyclic heterocyclic ring having 1-2 heteroatoms; or
   an R⁶ group and its adjacent R⁵ group are optionally taken together with their intervening atoms to form a 4-11 membered optionally substituted saturated or partially unsaturated monocyclic heterocyclic ring having 1-2 heteroatoms;
L¹ is a trivalent linker moiety that connects L and

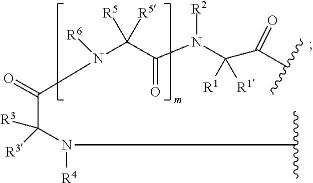

each m and n is independently 1, 2, 3, 4, 5, 6, 7, 8, 9, or 10; and
each heteroatom of the ABM is independently selected from nitrogen, oxygen, and sulfur.

In any aspect or embodiment described herein, each L¹ is independently selected from:

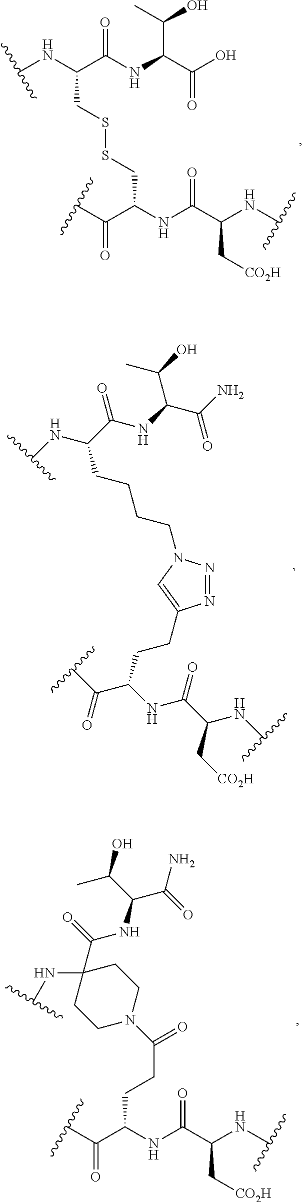 [SMILES: *N[C@@H](CC(=O)O)C(=O)N[C@@H](CSSC[C@H](N*)C(=O)N[C@H](C(=O)O)[C@@H](C)O)C(*)=O ; *N[C@@H](CC(=O)O)C(=O)N[C@@H](CCc1cn(CCCC[C@H](N*)C(=O)N[C@H](C(N)=O)[C@@H](C)O)nn1)C(*)=O ; *N[C@@H](CC(=O)O)C(=O)N[C@@H](CCC(=O)N1CCC(N*)(C(=O)N[C@H](C(N)=O)[C@@H](C)O)CC1)C(*)=O]

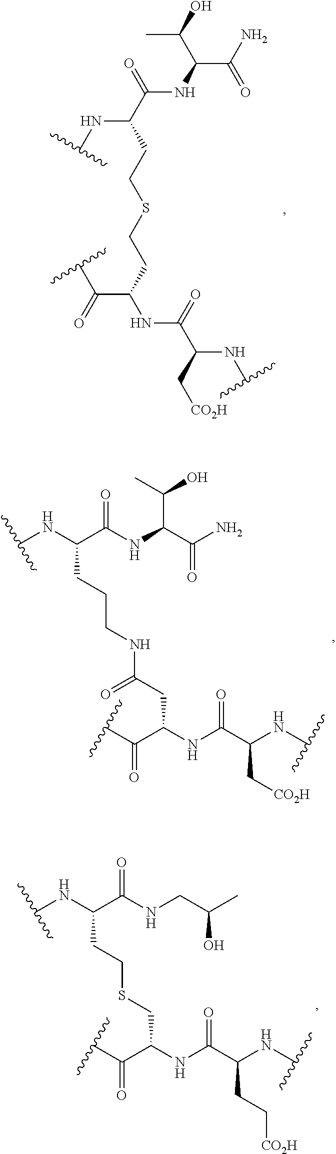 [SMILES: *N[C@@H](CC(=O)O)C(=O)N[C@@H](CCSCC[C@H](N*)C(=O)N[C@H](C(N)=O)[C@@H](C)O)C(*)=O ; *N[C@@H](CC(=O)O)C(=O)N[C@@H](CC(=O)NCCC[C@H](N*)C(=O)N[C@H](C(N)=O)[C@@H](C)O)C(*)=O ; *N[C@@H](CCSC[C@H](NC(=O)[C@H](CCC(=O)O)N*)C(*)=O)C(=O)NC[C@@H](C)O]

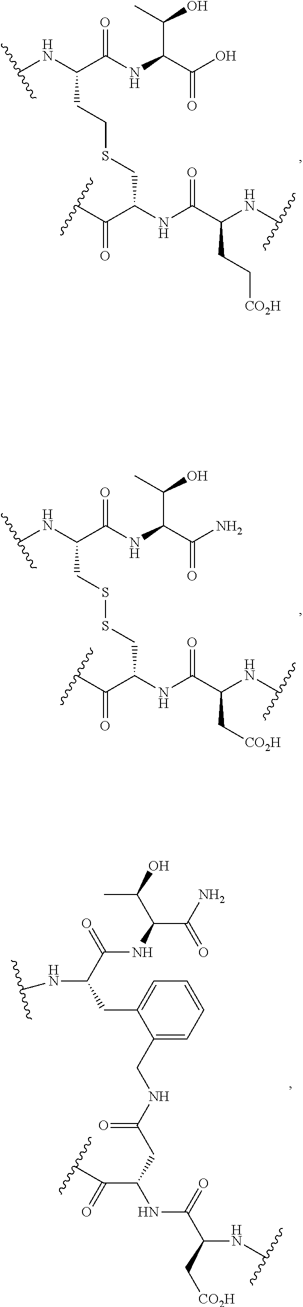 [SMILES: *N[C@@H](CCC(=O)O)C(=O)N[C@@H](CSCC[C@H](N*)C(=O)N[C@H](C(=O)O)[C@@H](C)O)C(*)=O ; *N[C@@H](CC(=O)O)C(=O)N[C@@H](CSSC[C@H](N*)C(=O)N[C@H](C(N)=O)[C@@H](C)O)C(*)=O ; *N[C@@H](CC(=O)O)C(=O)N[C@@H](CC(=O)NCc1ccccc1C[C@H](N*)C(=O)N[C@H](C(N)=O)[C@@H](C)O)C(*)=O]

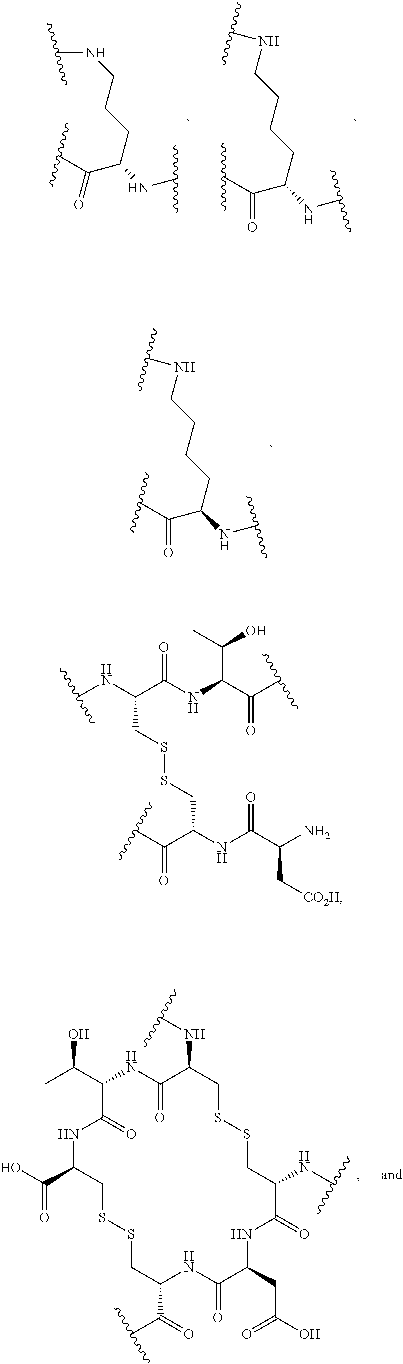 [SMILES: *NCCC[C@H](N*)C(*)=O ; *NCCCC[C@H](N*)C(*)=O ; *NCCCC[C@@H](N*)C(*)=O ; *N[C@@H](CSSC[C@H](NC(=O)[C@@H](N)CC(=O)O)C(*)=O)C(=O)N[C@H](C(*)=O)[C@@H](C)O ; *N[C@H]1CSSC[C@H](N*)C(=O)N[C@@H]([C@@H](C)O)C(=O)N[C@H](C(=O)O)CSSC[C@@H](C(*)=O)NC(=O)[C@H](CC(=O)O)NC1=O]

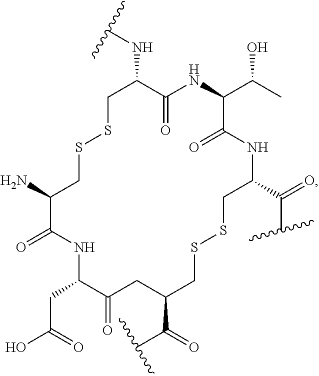 [SMILES: *N[C@H]1CSSC[C@H](N)C(=O)N[C@@H](CC(=O)O)C(=O)C[C@H](C(*)=O)CSSC[C@@H](C(*)=O)NC(=O)[C@H]([C@@H](C)O)NC1=O]

wherein the N-terminus is optionally acetylated (—COCH₃) and the C-terminus is optionally amidated (—NH₂).

In any aspect or embodiment described herein, each L¹ is independently selected from:

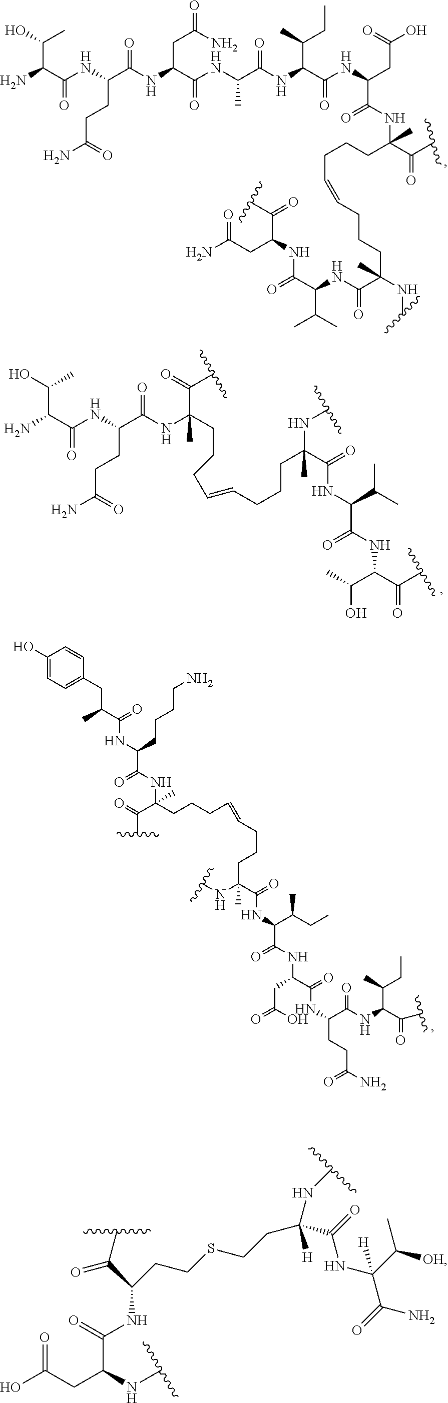

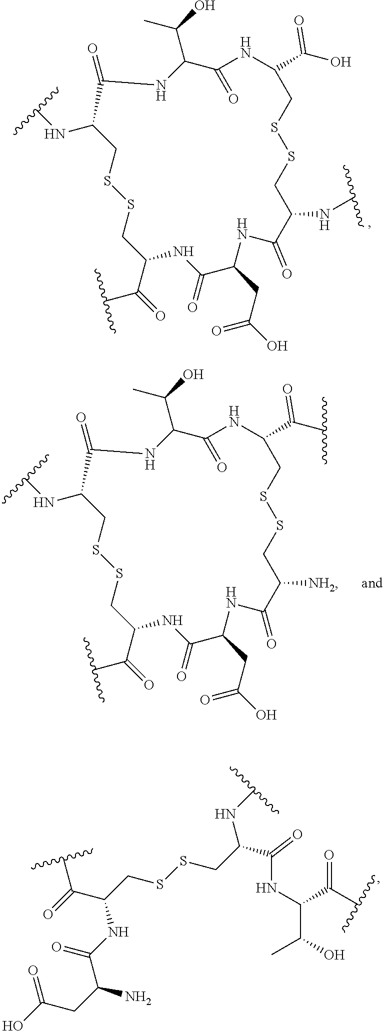

wherein the N-terminus is optionally acetylated (—COCH₃) and the C-terminus is optionally amidated (—NH₂).

In any aspect or embodiment described herein, a universal antibody binding moiety has the structure of

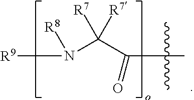

or salt form thereof, wherein:
each R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms, a 5-6 membered monocyclic heteroaromatic ring having 1-4 heteroatoms, or an 8-10 membered bicyclic heteroaromatic ring having 1-5 heteroatoms; or:
R⁷ and R^{7′} are optionally taken together with their intervening carbon atom to form a 3-8 membered optionally substituted saturated or partially unsaturated spirocyclic carbocyclic ring, or a 3-8 membered optionally substituted saturated or partially unsaturated spirocyclic heterocyclic ring having 1-2 heteroatoms;
each R^{7′} is independently hydrogen or optionally substituted C₁₋₃ aliphatic;
each R⁸ is independently hydrogen, or optionally substituted C₁₋₄ aliphatic, or:
R⁸ and its adjacent R⁷ group are optionally taken together with their intervening atoms to form a 4-11 membered optionally substituted saturated or partially unsaturated monocyclic heterocyclic ring having 1-2 heteroatoms;
each R⁹ is independently hydrogen, optionally substituted C₁₋₃ aliphatic, or —C(O)-(optionally substituted C₁₋₃ aliphatic);
o is 1, 2, 3, 4, 5, 6, 7, 8, 9, or 10; and
each heteroatom of the ABM is independently selected from nitrogen, oxygen, and sulfur.

In any aspect or embodiment described herein, one or more -(Xaa)z- is or comprises [X¹]ₚ₁[X²]ₚ₂-X³X⁴X⁵X⁶X⁷X⁸X⁹X¹⁰X¹¹X¹²-[X¹³]ₚ₁₃[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is an independently selected amino acid residue (e.g., of an amino acid of formula NH(R^{a1})-L^{a1}-C(R^{a2})(R^{a3})-L^{a2}-COOH), and each of p1, p2, p13, p14, p15 and p16 is independently 0, 1, 2, 3, 4, 5, 6, 7, 8, 9, or 10. In any aspect or embodiment described herein, each of X¹, X², X³, X⁴, X⁵, X⁶, X⁷, X⁸, X⁹, X¹⁰, X¹¹, X¹², and X¹³ is an independently selected amino acid residue of formula NH(R^{a1})-L^{a1}-C(R^{a2})(R^{a3})-L^{a2}-COOH. In any aspect or embodiment described herein, each of X¹, X², X³, X⁴, X⁵, X⁶, X⁷, X⁸, X⁹, X¹⁰, X¹¹, X¹², and X¹³ is an independently proteinogenic (natural) amino acid residue. In any aspect or embodiment described herein, one or more of X¹, X², X³, X⁴, X⁵, X⁶, X⁷, X⁸, X⁹, X¹⁰, X¹¹, X¹², and X¹³ are an independently selected non-proteinogenic (unnatural) amino acid residue as described herein.

In any aspect or embodiment described herein, an ABM peptide unit comprises a functional group in an amino acid residue that can react with a functional group of another amino acid residue. In any aspect or embodiment described herein, an ABM peptide unit comprises an amino acid residue with a side chain that comprises a functional group that can react with another functional group of the side chain of another amino acid residue to form a linkage (e.g., see compounds in EXAMPLES). In any aspect or embodiment described herein, one functional group of one amino acid residue is connected to a functional group of another amino acid residue to form a linkage (or bridge). For example, linkages are bonded to backbone atoms of ABM peptide units and comprise no backbone atoms. In any aspect or embodiment described herein, an ABM peptide unit comprises a linkage formed by two side chains of non-neighboring amino acid residues. In any aspect or embodiment described herein, a linkage is bonded to two backbone atoms of two non-neighboring amino acid residues. In any aspect or embodiment described herein, both backbone atoms bonded to a linkage are carbon atoms. In any aspect or embodiment described herein, a linkage has the structure of L^{b}, wherein each L^{b} is independently an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, -, -Cy-, —O—, —S—, —S—S—, —N(R′)—, —C(O)—, —C₂—, —S(O)₂N(R′)—, —C(O)S—, or —C(O)O—; each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon; each R′ is independently —R, —C(O)R, —CO₂R, or —SO₂R; each R is independently —H, or an optionally substituted group selected from C₁₋₃₀ aliphatic, C₁₋₃₀ heteroaliphatic having 1-10 heteroatoms, C₁₋₃₀ aryl, C₁₋₃₀ arylaliphatic, C₁₋₃₀ arylheteroaliphatic having 1-10 heteroatoms, 5-30 membered heteroaryl having 1-10 heteroatoms, and 3-30 membered heterocyclyl having 1-10 heteroatoms; or two R groups are optionally and independently taken together to form a covalent bond; or two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon; and each heteroatom of L^{b} is independently selected from oxygen, nitrogen, sulfur, phosphorus, and silicon. In any aspect or embodiment described herein, L^{b} is not a covalent bond. In any aspect or embodiment described herein, L^{b} comprises -Cy-. In any aspect or embodiment described herein, L^{b} comprises -Cy-, wherein -Cy- is optionally substituted heteroaryl. In any aspect or embodiment described herein, -Cy- is

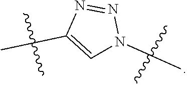 [SMILES: *c1cn(*)nn1]

In any aspect or embodiment described herein, L^{b} is

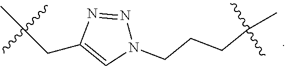 [SMILES: *CCCn1cc(C*)nn1]

In any aspect or embodiment described herein, such an L^{b} can be formed by a —N₃ group of the side chain of one amino acid residue, and the [symbol] of the side chain of another amino acid residue. In any aspect or embodiment described herein, a linkage is formed through connection of two thiol groups (e.g., thiol groups from two cysteine residues). In any aspect or embodiment described herein, L^{b} comprises —S—S—. In any aspect or embodiment described herein, L^{b} is —CH₂—S—S—CH₂—. In any aspect or embodiment described herein, a linkage is formed through connection of an amino group (e.g., —NH₂ in the side chain of a lysine residue) and a carboxylic acid group (e.g., —COOH in the side chain of an aspartic acid or glutamic acid residue). In any aspect or embodiment described herein, L^{b} comprises —C(O)—N(R′)—. In any aspect or embodiment described herein, L^{b} comprise —C(O)—NH—. In any aspect or embodiment described herein, L^{b} is —CH₂CONH—(CH₂)₃—. In any aspect or embodiment described herein, L^{b} comprises —C(O)—N(R′)—, wherein R′ is R, and is taken together with an R group on the peptide backbone to form a ring. In any aspect or embodiment described herein, L^{b} is —(CH₂)₂—N(R′)—CO—(CH₂)₂-. In any aspect or embodiment described herein, -Cy- is an optionally substituted phenylene. In any aspect or embodiment described herein, -Cy- is an optionally substituted 1,2-phenylene. In any aspect or embodiment described herein, L^{b} is an optionally substituted

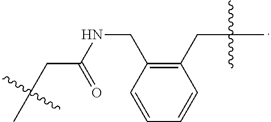 [SMILES: *CC(=O)NCc1ccccc1C*]

In any aspect or embodiment described herein, L^{b} is an optionally substituted

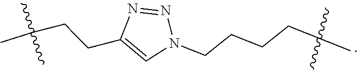 [SMILES: *CCCCn1cc(CC*)nn1]

In any aspect or embodiment described herein, L^{b} is an optionally substituted bivalent C₂₋₂₀ bivalent aliphatic. In any aspect or embodiment described herein, L^{b} is an optionally substituted —(CH₂)₉-CH═CH—(CH₂)₉-. In any aspect or embodiment described herein, L^{b} is an optionally substituted —(CH₂)₃-CH═CH—(CH₂)₃--.

In any aspect or embodiment described herein, two amino acid residues bonded to a linkage are separated by 1, 2, 3, 4, 5, 6, 7, 8, 9, 10, 11, 12, 13, 14, 15, or more amino acid residues between them (excluding the two amino acid residues bonded to the linkage). In any aspect or embodiment described herein, the number is 1 amino acid. In any aspect or embodiment described herein, the number is 2 amino acids. In any aspect or embodiment described herein, the number is 3 amino acids. In any aspect or embodiment described herein, the number is 4 amino acids. In any aspect or embodiment described herein, the number is 5 amino acids. In any aspect or embodiment described herein, the number is 6 amino acids. In any aspect or embodiment described herein, the number is 7 amino acids. In any aspect or embodiment described herein, the number is 8 amino acids. In any aspect or embodiment described herein, the number is 9 amino acids. In any aspect or embodiment described herein, the number is 10 amino acids. In any aspect or embodiment described herein, the number is 11 amino acids. In any aspect or embodiment described herein, the number is 12 amino acids. In any aspect or embodiment described herein, the number is 13 amino acids. In any aspect or embodiment described herein, the number is 14 amino acids. In any aspect or embodiment described herein, the number is 15 amino acids.

In any aspect or embodiment described herein, each of p1, p2, p13, p14, p15 and p16 is 0. In any aspect or embodiment described herein, one or more -(Xaa)z- is or comprises —X³X⁴X⁵X⁶X⁷X⁸X⁹X¹⁰X¹¹X¹²— wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.

In any aspect or embodiment described herein, each of X³, X⁴, X⁵, X⁶, X⁷, X⁸, X⁹, X¹⁰, X¹¹, and X¹² is independently an amino acid residue of formula NH(R^{a1})-L^{a1}-C(R^{a2})(R^{a3})-L^{a2}-COOH. In any aspect or embodiment described herein, X⁵ is Xaa^{A} or Xaa^{P}. In any aspect or embodiment describe herein, X⁵ is Xaa^{A}. In any aspect or embodiment described herein, X⁵ is Xaa^{P}. In any aspect or embodiment described herein, X⁵ is an amino acid residue whose side chain comprises an optionally substituted saturated, partially saturated or aromatic ring. In any aspect or embodiment described herein, X⁵ is

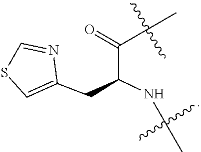 [SMILES: *N[C@@H](Cc1cscn1)C(*)=O]

In any aspect or embodiment described herein, X⁵ is

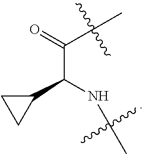 [SMILES: *N[C@H](C(*)=O)C1CC1]

In any aspect or embodiment described herein, X⁶ is Xaa^{A}. In any aspect or embodiment described herein, X⁶ is Xaa^{P}. In any aspect or embodiment described herein, X⁶ is His. In any aspect or embodiment described herein, X¹² is Xaa^{A}. In any aspect or embodiment described herein, X¹² is Xaa^{P}. In some embodiments, X⁹ is Asp.

In some embodiments, X⁹ is Glu. In any aspect or embodiment described herein, X¹² is

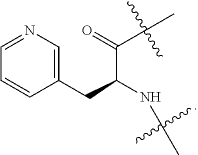 [SMILES: *N[C@@H](Cc1cccnc1)C(*)=O]

In any aspect or embodiment described herein, X¹² is

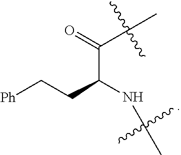 [SMILES: *N[C@@H](CCc1ccccc1)C(*)=O]

In any aspect or embodiment described herein, each of X⁷, X¹⁰, and X¹¹ is independently an amino acid residue with a hydrophobic side chain (“hydrophobic amino acid residue”, Xaa^{H}). In any aspect or embodiment described herein, X⁷ is Xaa^{H}. In any aspect or embodiment described herein, X⁷ is

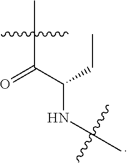 [SMILES: *N[C@@H](CC)C(*)=O]

In any aspect or embodiment described herein, X⁷ is Val. In any aspect or embodiment described herein, X¹⁰ is Xaa^{H}. In any aspect or embodiment described herein, X¹⁰ is Met. In any aspect or embodiment described herein, X¹⁰ is

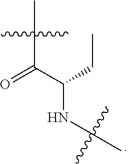 [SMILES: *N[C@@H](CC)C(*)=O]

In any aspect or embodiment described herein, X¹¹ is Xaa^{H}. In any aspect or embodiment described herein, X¹¹ is

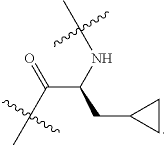 [SMILES: *N[C@@H](CC1CC1)C(*)=O]

In any aspect or embodiment described herein, X⁸ is Gly. In any aspect or embodiment described herein, X⁴ is Pro. In any aspect or embodiment described herein, X³ is Lys. In any aspect or embodiment described herein, the—COOH of X¹² forms an amide bond with the side chain amino group of Lys (X³), and the other amino group of the Lys (X³) is connected to a linker moiety and then a target binding moiety.

In any aspect or embodiment described herein, -(Xaa)z- is or comprises —X³X⁴X⁵X⁶X⁷X⁸X⁹X¹⁰X¹¹X¹²— wherein:
each of X³, X⁴, X⁵, X⁶, X⁷, X⁸, X⁹X¹⁰, X¹¹, and X¹² is an independently selected amino acid residue;
at least two amino acid residues are connected through one or more linkages of L^{b};
L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.

In any aspect or embodiment described herein, each of X³, X⁴, X⁵, X⁶, X⁷, X⁸, X⁹, X¹⁰, X¹¹, and X¹² is an independently an amino acid of formula NH(RarLaulC(R^{a2})(R^{a3})-L^{a2}-COOH. In any aspect or embodiment described herein, two non-neighboring amino acid residues are connected by L^{b}. In any aspect or embodiment described here, X⁵ and X¹⁰ are connected by L. In any aspect or embodiment described here, there is one linkage L^{b}. In any aspect or embodiment described here, X⁶ is Xaa^{A}. In any aspect or embodiment described here, X⁶ is Xaa^{P}. In any aspect or embodiment described here, X⁶ is His. In some embodiments, X⁹ is Asp. In some embodiments, X⁹ is Glu. In any aspect or embodiment described here, X¹² is Xaa^{A}. In any aspect or embodiment described here, X¹² is

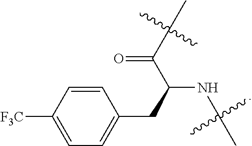 [SMILES: *N[C@@H](Cc1ccc(C(F)(F)F)cc1)C(*)=O]

In any aspect or embodiment described here, X¹² is

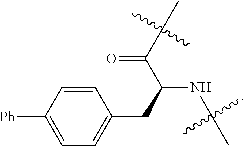 [SMILES: *N[C@@H](Cc1ccc(-c2ccccc2)cc1)C(*)=O]

In any aspect or embodiment described here, X¹² is N

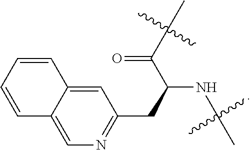 [SMILES: *N[C@@H](Cc1cc2ccccc2cn1)C(*)=O]

In any aspect or embodiment described here, each of X⁴, X⁷, and X¹¹ is independently selected from Xaa^{H}. In any aspect or embodiment described here, X⁴ is Xaa^{H}. In any aspect or embodiment described here, X⁴ is Ala. In any aspect or embodiment described here, X⁷ is Xaa^{H}. In any aspect or embodiment described here, X⁷ is

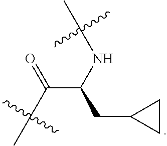 [SMILES: *N[C@@H](CC1CC1)C(*)=O]

In any aspect or embodiment described here, X¹¹ is Xaa^{H}. In any aspect or embodiment described here, X¹¹ is

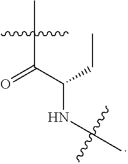 [SMILES: *N[C@@H](CC)C(*)=O]

In any aspect or embodiment described here, X⁸ is Gly. In any aspect or embodiment described here, X³ is Lys. In any aspect or embodiment described here, the —COOH of X¹² forms an amide bond with the side chain amino group of Lys (X³), and the other amino group of the Lys (X³) is connected to a linker moiety and then a target binding moiety. In any aspect or embodiment described here, L^{b} is an optionally substituted

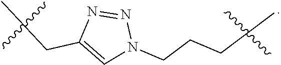 [SMILES: *CCCn1cc(C*)nn1]

In any aspect or embodiment described here, L^{b} is an optionally substituted

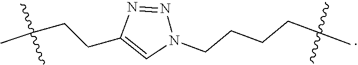 [SMILES: *CCCCn1cc(CC*)nn1]

In any aspect or embodiment described here, L^{b} connects two alpha-carbon atoms of two different amino acid residues. In any aspect or embodiment described here, both X⁵ and X¹⁰ are Cys, and the two —SH groups of their side chains form —S—S— (e.g., L^{b} is —CH₂—S—S—CH₂—).

In any aspect or embodiment described here, -(Xaa)z- is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰, X¹¹, and X¹² is an independently selected amino acid residue;
at least two amino acid residues are connected through one or more linkages of L^{b}; L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.

In any aspect or embodiment described herein, each of X², X³, X⁴, X⁵, X⁶, X⁷, X⁸, X⁹, X¹⁰, X¹¹, and X¹² is an independently selected amino acid of formula NH(R^{a1})-L^{a1}C(R^{a2})(R^{a3})-L^{a2}-COOH as described in the present disclosure. In any aspect or embodiment described herein, two non-neighboring amino acid residues are connected by L^{b}. In any aspect or embodiment described herein, there is one linkage L^{b}. In any aspect or embodiment described herein, X² and X¹² are connected by L^{b}. In any aspect or embodiment described herein, L^{b} is an optionally substituted —CH₂—S—S—CH₂—. In any aspect or embodiment described herein, L^{b} is an optionally substituted —CH₂—CH₂—S—CH₂—. In any aspect or embodiment described herein, L^{b} is an optionally substituted

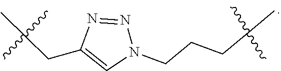 [SMILES: *CCCn1cc(C*)nn1]

In any aspect or embodiment described herein, L^{b} is an optionally substituted

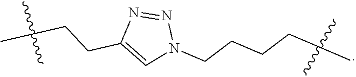 [SMILES: *CCCCn1cc(CC*)nn1]

In any aspect or embodiment described herein, L^{b} is an optionally substituted —CH₂CH₂CO—N(R′)—CH₂CH₂—. In any aspect or embodiment described herein, R′ are taken together with an R group on the backbone atom that —N(R′)—CH₂CH₂— is bonded to form a ring. In any aspect or embodiment described herein, a formed ring is a 3-, 4-, 5-, 6-, 7- or 8-membered ring. In any aspect or embodiment described herein, a formed ring is monocyclic. In any aspect or embodiment described herein, a formed ring is saturated. In any aspect or embodiment described herein, L^{b} is an optionally substituted

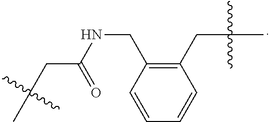 [SMILES: *CC(=O)NCc1ccccc1C*]

In any aspect or embodiment described herein, L^{b} connects two alpha-carbon atoms of two different amino acid residues. In any aspect or embodiment described herein, X⁴ is Xaa^{A}. In any aspect or embodiment described herein, X⁴ is Tyr. In any aspect or embodiment described herein, X⁵ is Xaa^{A}. In any aspect or embodiment described herein, X⁵ is Xaa^{P}. In any aspect or embodiment described herein, X⁵ is His. In any aspect or embodiment described herein, X⁸ is Asp. In any aspect or embodiment described herein, X⁸ is Glu. In any aspect or embodiment described herein, X¹¹ is Tyr. In any aspect or embodiment described herein, both X² and X¹² are Cys, and the two —SH groups of their side chains form —S—S— (e.g., L^{b} is —CH₂—S—S—CH₂—). In any aspect or embodiment described herein, each of X³, X⁶, X⁹, and X¹⁰ is an independently selected Xaa^{H}. In any aspect or embodiment described herein, X³ is Xaa^{H}. In any aspect or embodiment described herein, X³ is Ala. In any aspect or embodiment described herein, X⁶ is Xaa^{H}. In any aspect or embodiment described herein, X⁶ is Leu. In any aspect or embodiment described herein, X⁹ is Xaa^{H}. In any aspect or embodiment described herein, X⁹ is Leu. In any aspect or embodiment described herein, X⁹ is

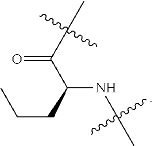 [SMILES: *N[C@@H](CCC)C(*)=O]

In any aspect or embodiment described herein, X¹⁰ is Xaa^{H}. In any aspect or embodiment described herein, X¹⁰ is Val. In any aspect or embodiment described herein, X¹⁰ is

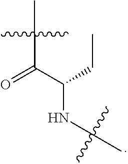 [SMILES: *N[C@@H](CC)C(*)=O]

In any aspect or embodiment described herein, X⁷ is Gly. In any aspect or embodiment described herein, p1 is 1. In any aspect or embodiment described herein, X¹ is Asp. In any aspect or embodiment described herein, p13 is 1. In any aspect or embodiment described herein, p14, p15 and p16 are 0. In any aspect or embodiment described herein, X¹³ is an amino acid residue comprising a polar uncharged side chain (e.g., at physiological pH, “polar uncharged amino acid residue”, Xaa^{L}). In any aspect or embodiment described herein, X¹³ is Val. In any aspect or embodiment described herein, p13 is 0. In any aspect or embodiment described herein, R^{c} is —NHCH₂CH(OH)CH₃. In any aspect or embodiment described herein, R^{c} is (R)—NHCH₂CH(OH)CH₃. In any aspect or embodiment described herein, R^{c} is (S)—NHCH₂CH(OH)CH₃.

In any aspect or embodiment described herein, -(Xaa)z- is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰, X¹¹, and X¹² is an independently selected amino acid residue;
at least two amino acid residues are connected through one or more linkages of L^{b}; L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.

In any aspect or embodiment described herein, each of X², X³, X⁴, X⁵, X⁶, X⁷, X⁸, X⁹, X¹⁰, X¹¹, and X¹² is an independently selected amino acid of formula NH(R^{a1})-L^{a1}-C(R^{a2})(R^{a3})-L^{a2}-COOH. In any aspect or embodiment described herein, two non-neighboring amino acid residues are connected by L^{b}. In any aspect or embodiment described herein, there is one linkage L^{b}. In any aspect or embodiment described herein, there are two or more linkages L^{b}. In any aspect or embodiment described herein, there are two linkages L^{b}. In any aspect or embodiment described herein, X² and X¹² are connected by L^{b}. In any aspect or embodiment described herein, X⁴ and X⁹ are connected by L^{b}. In any aspect or embodiment described herein, X⁴ and X¹⁰ are connected by L^{b}. In any aspect or embodiment described herein, L^{b} is —CH₂—S—S—CH₂—. In any aspect or embodiment described herein, L^{b} is an optionally substituted

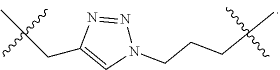 [SMILES: *CCCn1cc(C*)nn1]

In any aspect or embodiment described herein, L^{b} is an optionally substituted

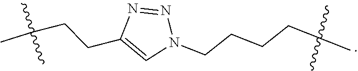 [SMILES: *CCCCn1cc(CC*)nn1]

In any aspect or embodiment described herein, both X² and X¹² are Cys, and the two —SH groups of their side chains form —S—S— (e.g., L^{b} is —CH₂—S—S—CH₂—). In any aspect or embodiment described herein, both X⁴ and X¹⁰ are Cys, and the two —SH groups of their side chains form —S—S— (e.g., L^{b} is —CH₂—S—S—CH₂—). In any aspect or embodiment described herein, X⁴ and X⁹ are connected by L^{b}, wherein L^{b} is an optionally substituted

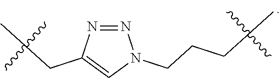 [SMILES: *CCCn1cc(C*)nn1]

In any aspect or embodiment described herein, X⁴ and X⁹ are connected by L^{b}, wherein L^{b} is an optionally substituted

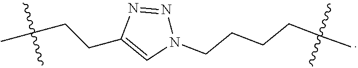 [SMILES: *CCCCn1cc(CC*)nn1]

In any aspect or embodiment described herein, X⁵ is Xaa^{A}. In any aspect or embodiment described herein, X⁵ is Xaa^{P}. In any aspect or embodiment described herein, X⁵ is His. In any aspect or embodiment described herein, X⁸ is Asp. In any aspect or embodiment described herein, X⁸ is Glu. In any aspect or embodiment described herein, X¹¹ is Tyr. In some embodiments, X¹¹ is an optionally substituted N

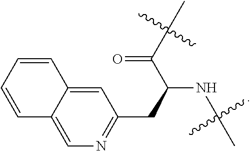 [SMILES: *N[C@@H](Cc1cc2ccccc2cn1)C(*)=O]

In any aspect or embodiment described herein, X² and X¹² are connected by L^{b}, wherein L^{b} is —CH₂—S—CH₂CH₂—. In any aspect or embodiment described herein, L^{b} connects two alpha-carbon atoms of two different amino acid residues. In any aspect or embodiment described herein, each of X³, X⁶, and X⁹ is independently selected from Xaa^{H}. In any aspect or embodiment described herein, X³ is Xaa^{H}. In any aspect or embodiment described herein, X³ is Ala. In any aspect or embodiment described herein, X⁶ is Xaa^{H}. In any aspect or embodiment described herein, X⁶ is Leu. In any aspect or embodiment described herein, X⁶ is

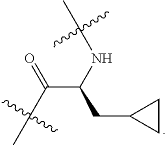 [SMILES: *N[C@@H](CC1CC1)C(*)=O]

In any aspect or embodiment described herein, X⁹ is Xaa^{H}. In any aspect or embodiment described herein, X⁹ is Leu. In any aspect or embodiment described herein, X⁹ is

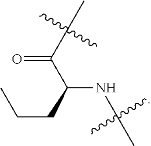 [SMILES: *N[C@@H](CCC)C(*)=O]

In any aspect or embodiment described herein, X¹⁰ is Xaa^{H}. In any aspect or embodiment described herein, X¹⁰ is Val. In any aspect or embodiment described herein, X⁷ is Gly. In any aspect or embodiment described herein, p1 is 1. In any aspect or embodiment described herein, X¹ is Xaa^{N}. In any aspect or embodiment described herein, X¹ is Asp. In any aspect or embodiment described herein, X¹ is Glu.

In any aspect or embodiment described herein, -(Xaa)z- is or comprises —X²X³X⁴X⁵X⁶X⁷X⁸X⁹X¹⁰X¹¹X¹²X¹³X¹⁴X¹⁵X¹⁶-, wherein:
each of X², X³, X⁴, X⁵, X⁶, X⁷, X⁸, X⁹, X¹⁰, X¹, X¹², X¹³, X¹⁴, X¹⁵, and X¹⁶ is an independently selected amino acid residue;
at least two amino acid residues are connected through a linkage of L^{b};
L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X³ is Xaa^{A}.

In any aspect or embodiment described herein, each of X², X³, X⁴, X⁵, X⁶, X⁷, X⁸, X⁹, X¹⁰, X¹, X¹², X³, X¹⁴, X, and X¹⁶ is an independently selected amino acid of formula NH(R^{a1})-L^{a1}-C(R^{a2})(R^{a3})-L^{a2}-COOH. In any aspect or embodiment described herein, two non-neighboring amino acid residues are connected by L^{b}. In any aspect or embodiment described herein, there is one linkage L^{b}. In any aspect or embodiment described herein, there are two or more linkages L^{b}. In any aspect or embodiment described herein, there are two linkages L^{b}. In any aspect or embodiment described herein, X² are connected to X¹⁶ by L^{b}. In any aspect or embodiment described herein, X⁴ are connected to X¹⁴ by L^{b}. In any aspect or embodiment described herein, both X² and X¹⁶ are Cys, and the two —SH groups of their side chains form —S—S— (e.g., L^{b} is —CH₂—S—S—CH₂—). In any aspect or embodiment described herein, both X⁴ and X¹⁴ are Cys, and the two —SH groups of their side chains form —S—S— (e.g., L^{b} is —CH₂—S—S—CH₂—). In any aspect or embodiment described herein, L^{b} connects two alpha-carbon atoms of two different amino acid residues. In any aspect or embodiment described herein, X³ is Asp. In any aspect or embodiment described herein, X³ is Glu. In any aspect or embodiment described herein, X⁵ is Xaa^{H}. In any aspect or embodiment described herein, X⁵ is Ala. In any aspect or embodiment described herein, X⁶ is Xaa^{A}. In any aspect or embodiment described herein, X⁶ is Tyr. In any aspect or embodiment described herein, X⁷ is Xaa^{A}. In any aspect or embodiment described herein, X⁷ is Xaa^{P}. In any aspect or embodiment described herein, X⁷ is His. In any aspect or embodiment described herein, X⁸ is Xaa^{H}. In any aspect or embodiment described herein, X⁸ is Ala. In any aspect or embodiment described herein, X⁹ is Gly. In any aspect or embodiment described herein, X¹⁰ is Asp. In any aspect or embodiment described herein, X¹⁰ is Glu. In any aspect or embodiment described herein, X¹¹ is Xaa^{H}. In any aspect or embodiment described herein, X¹¹ is Leu. In any aspect or embodiment described herein, X¹² is Xaa^{H}. In any aspect or embodiment described herein, X¹² is Val. In any aspect or embodiment described herein, X¹³ is Xaa^{A}. In any aspect or embodiment described herein, X¹³ is Tyr. In any aspect or embodiment described herein, X¹¹ is an amino acid residue comprising a polar uncharged side chain (e.g., at physiological pH, “polar uncharged amino acid residue”, Xaa^{L}). In any aspect or embodiment described herein, X¹¹ is Val. In any aspect or embodiment described herein, p1 is 1. In any aspect or embodiment described herein, X¹ is Xaa^{N}. In any aspect or embodiment described herein, X¹ is Asp. In any aspect or embodiment described herein, X¹ is Glu.

As appreciated by those skilled in the art, in any aspect or embodiment described herein, an amino acid residue may be replaced by another amino acid residue having similar properties, e.g., one Xaa^{H}(e.g., Val, Leu, etc.) may be replaced with another Xaa^{H} (e.g., Leu, Ile, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In any aspect or embodiment described herein, one or more ABM, independently, is or comprises an ABM from an exemplary compound of the present disclosure.

In any aspect or embodiment described herein, one or more ABM, independently, is or comprises an optionally substituted amino acid sequence selected from: APAR, PARA, HWRGWA, WGRR, RRGW, NRFRGKYK, NARKFYK, RHRFNKD, TY, TYK, RTY, RTYK, PAM, WHL, ELVW, AWHLGELVW, DCAWHLGELVWCT, D-Cyclo[CAWHLGELVWC]T, DpLpAWHLGELVW, DpLpDCAWHLGELVWCT, CDCAWHLGELVWCTC, Ac-ASYHLGELVW[1,2,3,4-tetrahydroisoquinoline-3-carboxylic acid][Aib]C, Cyclo[Ac-ASYHLGELVW[1,2,3,4-tetrahydroisoquinoline-3-carboxylic acid][Aib]C], Ac-ASYHLGELVW[1,2,3,4-tetrahydroisoquinoline-3-carboxylic acid][Aib]C[dE], Cyclo[Ac-ASYHLGELVW[1,2,3,4-tetrahydroisoquinoline-3-carboxylic acid][Aib]C][dE], Ac-ASYHLGELVW[1,2,3,4-tetrahydroisoquinoline-3-carboxylic acid][Aib]CE, and Cyclo[Ac-ASYHLGELVW[1,2,3,4-tetrahydroisoquinoline-3-carboxylic acid][Aib]C]E.

In any aspect or embodiment described herein, one or more ABM, independently, is or comprises an optionally substituted antibody binding moiety independently selected from Fc-III, Fc-III-4c, FcRM, FcBP-1, FcBP-2, FcRM, and PAM.

In any aspect or embodiment described herein, one or more ABM, independently, is or comprises an optionally substituted chemical structure selected from:

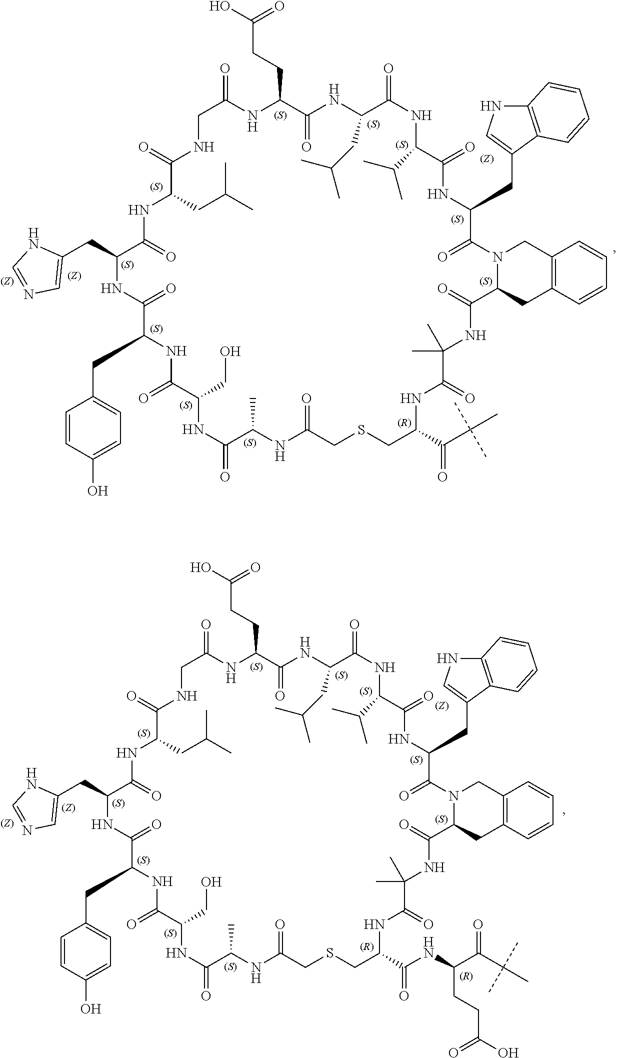

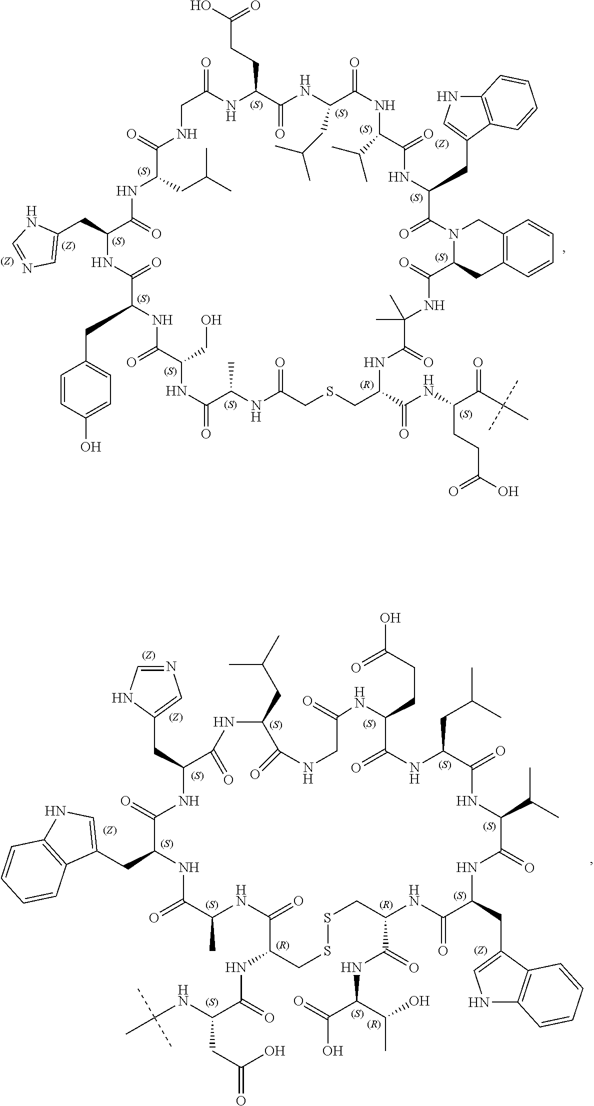

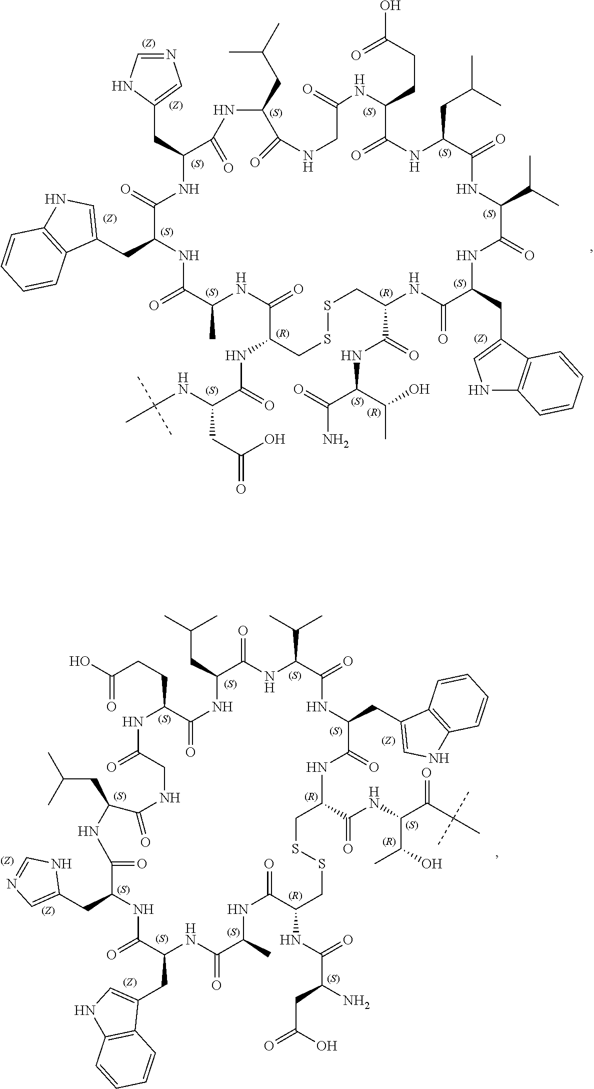

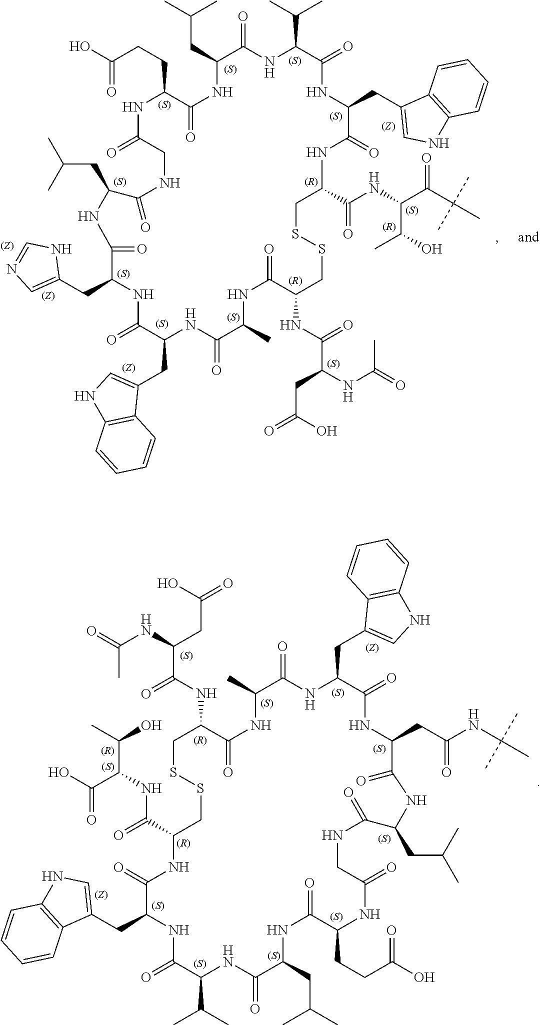

In any aspect or embodiment described herein, an antibody binding moiety, such as a universal antibody binding moiety, is a universal antibody binding moiety of an exemplified compound in the EXAMPLES section. In any aspect or embodiment described herein, an antibody binding moiety, such as a universal antibody binding moiety, is or comprises an optionally substituted chemical structure selected from:

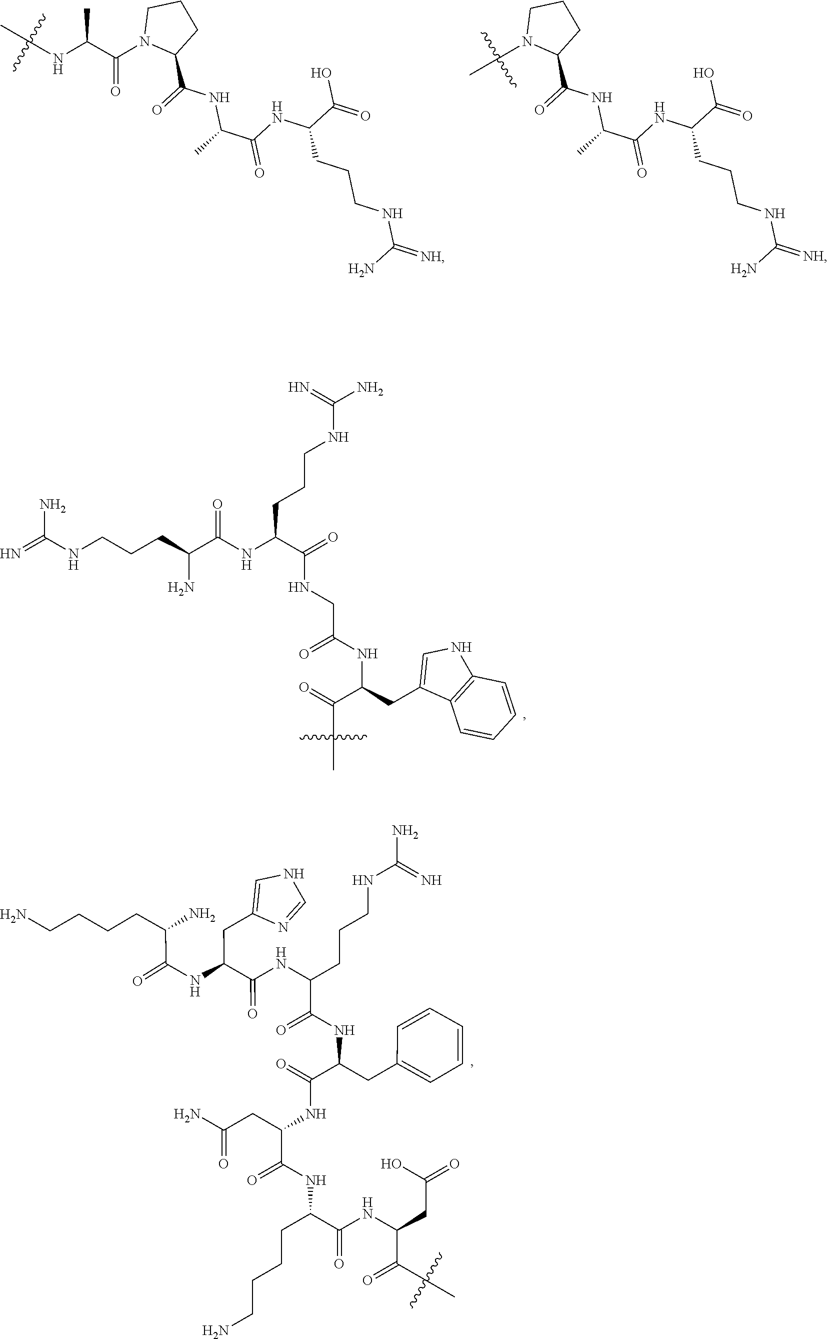 [SMILES: *N[C@@H](C)C(=O)N1CCC[C@H]1C(=O)N[C@@H](C)C(=O)N[C@@H](CCCNC(=N)N)C(=O)O ; *N1CCC[C@H]1C(=O)N[C@@H](C)C(=O)N[C@@H](CCCNC(=N)N)C(=O)O ; *C(=O)[C@H](Cc1c[nH]c2ccccc12)NC(=O)CNC(=O)[C@H](CCCNC(=N)N)NC(=O)[C@@H](N)CCCNC(=N)N ; *C(=O)[C@H](CC(=O)O)NC(=O)[C@H](CCCCN)NC(=O)[C@H](CC(N)=O)NC(=O)[C@H](Cc1ccccc1)NC(=O)C(CCCNC(=N)N)NC(=O)[C@H](Cc1c[nH]cn1)NC(=O)[C@@H](N)CCCCN]

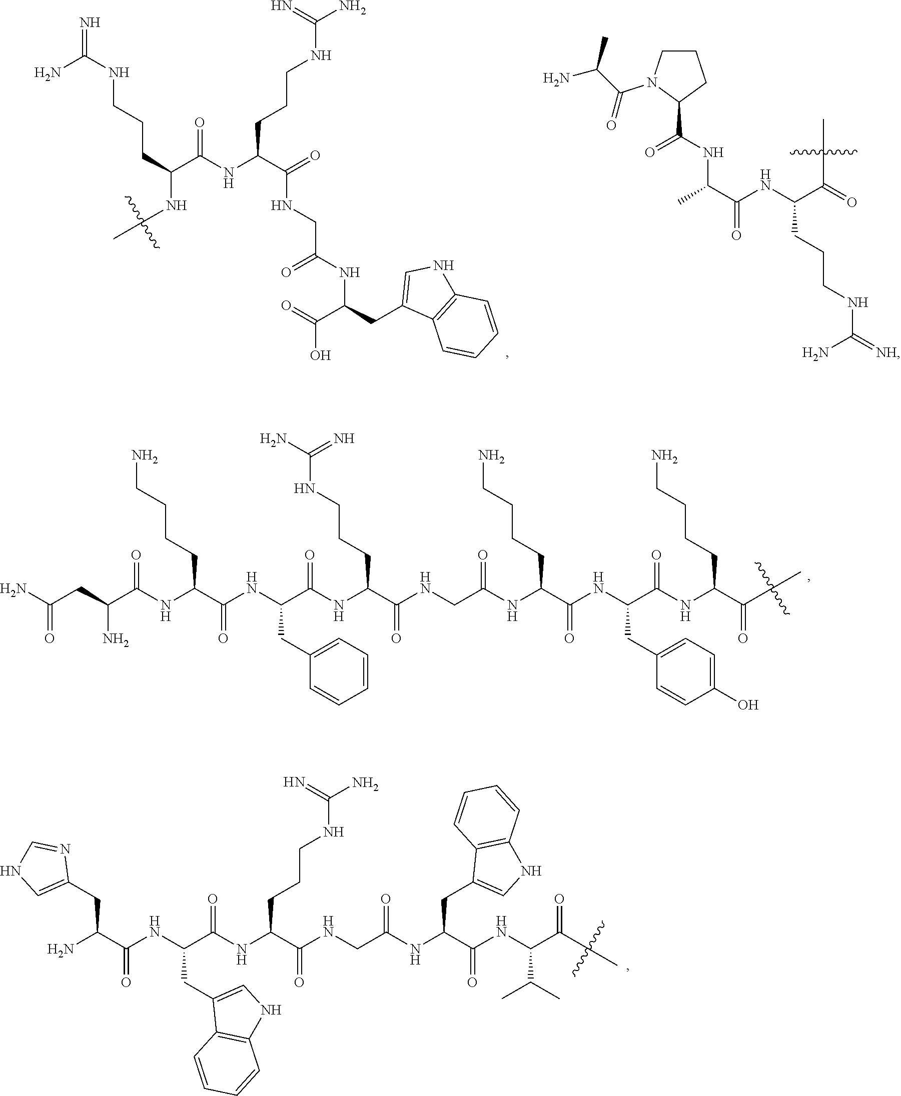 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O ; *C(=O)[C@H](CCCNC(=N)N)NC(=O)[C@H](C)NC(=O)[C@@H]1CCCN1C(=O)[C@H](C)N ; *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O ; *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C]

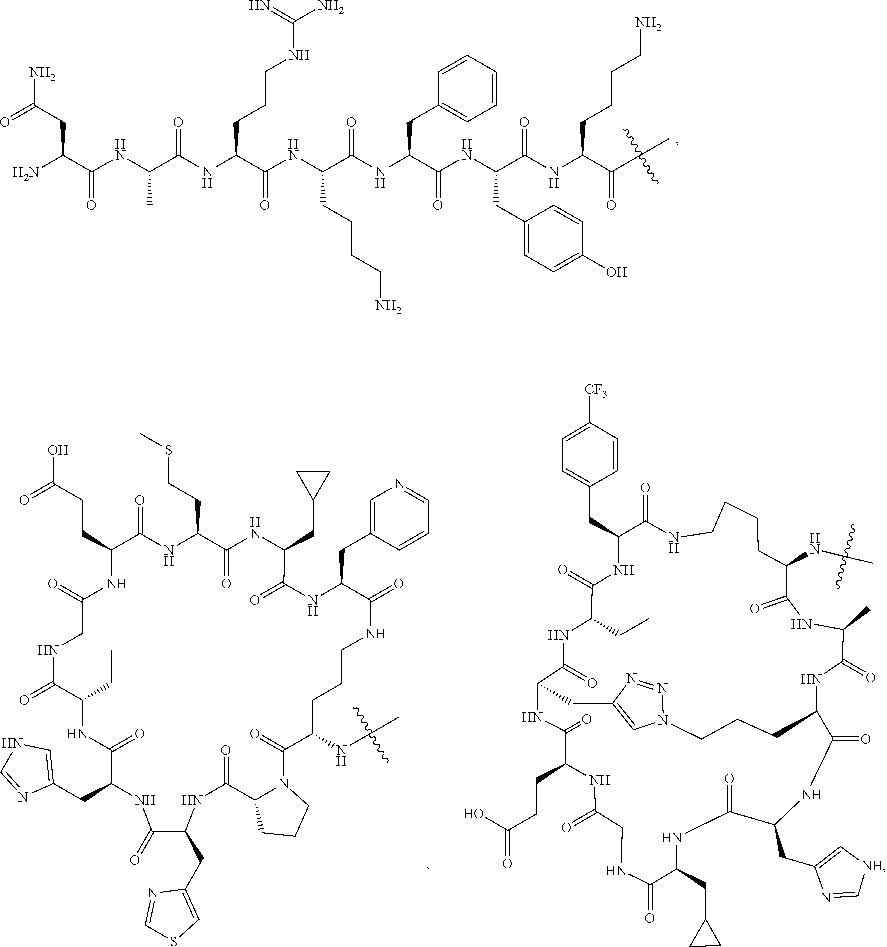

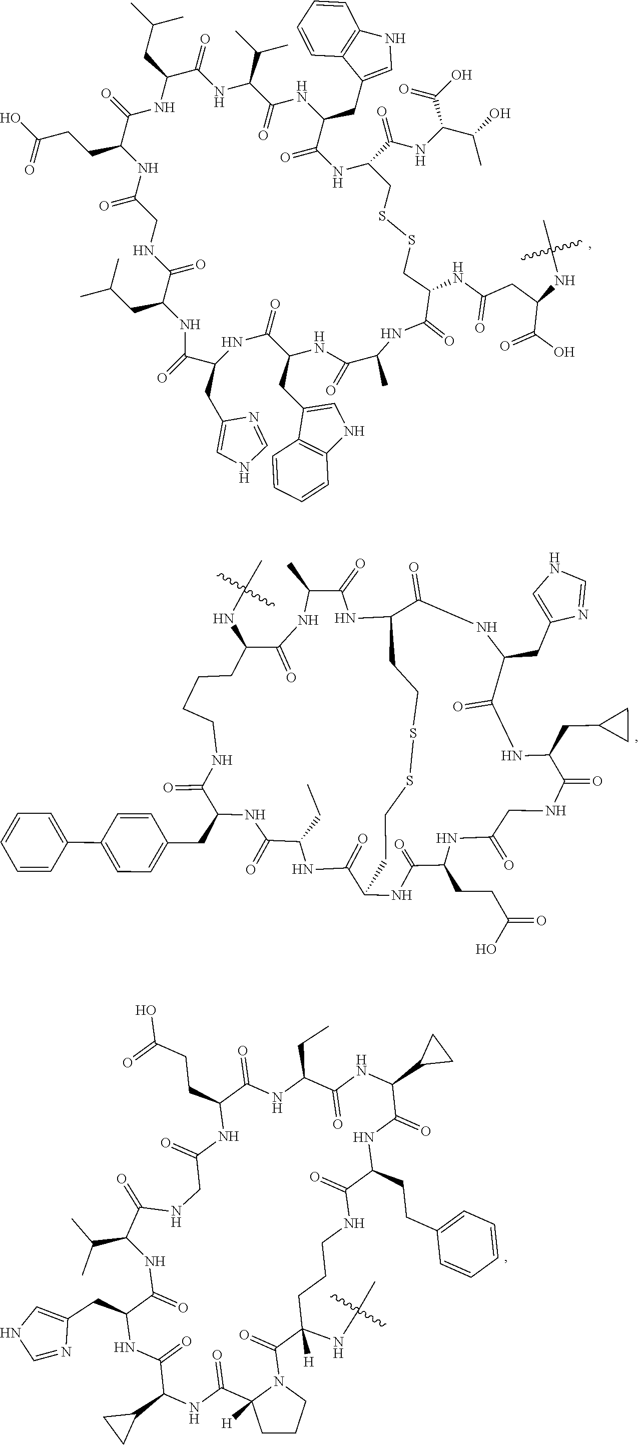

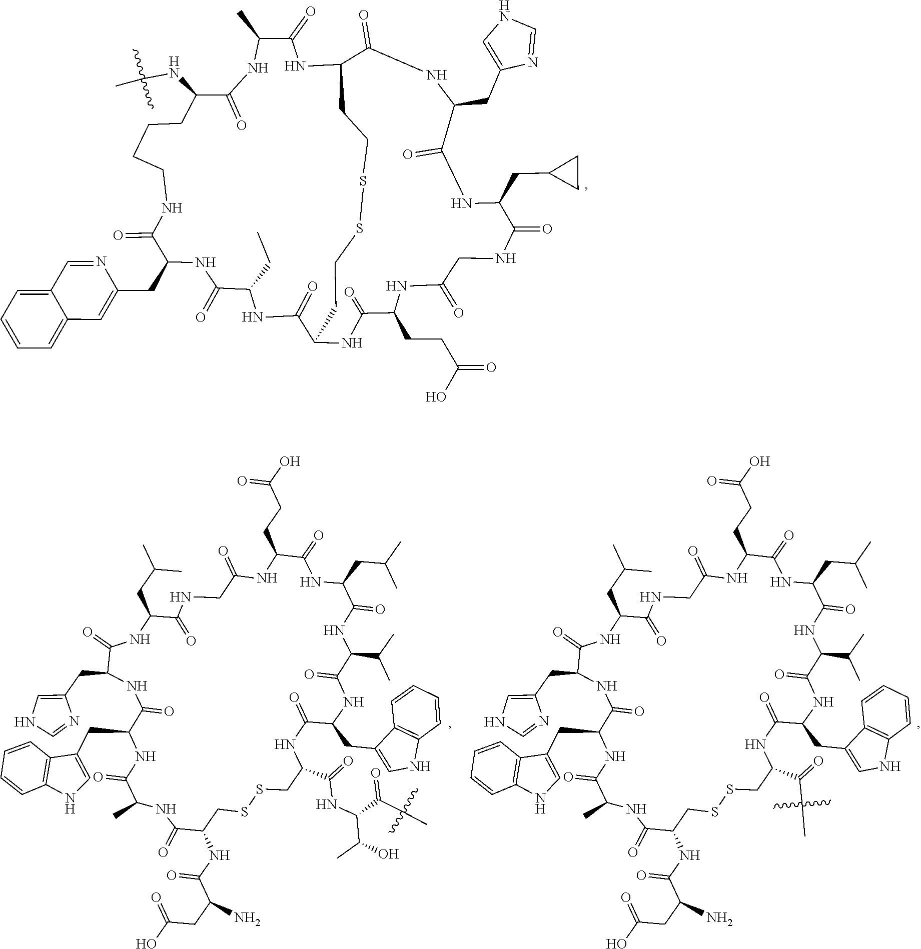

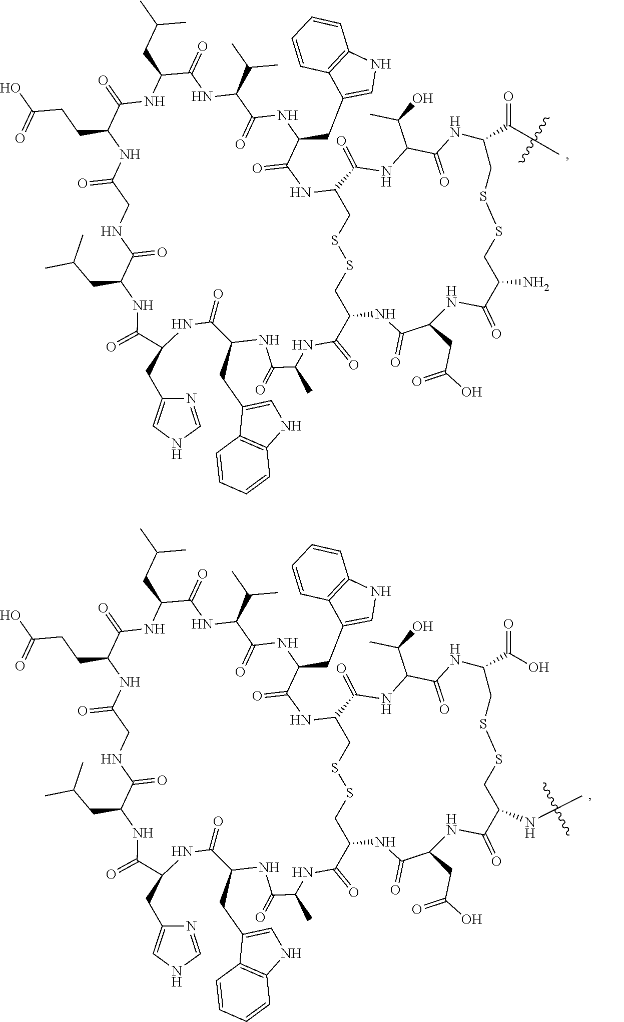

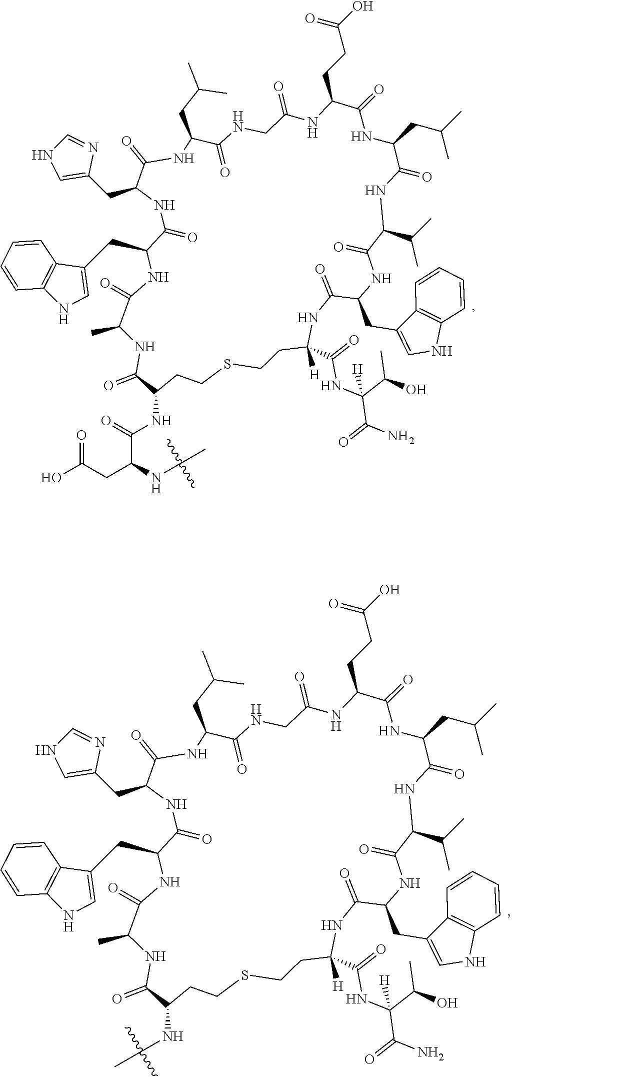

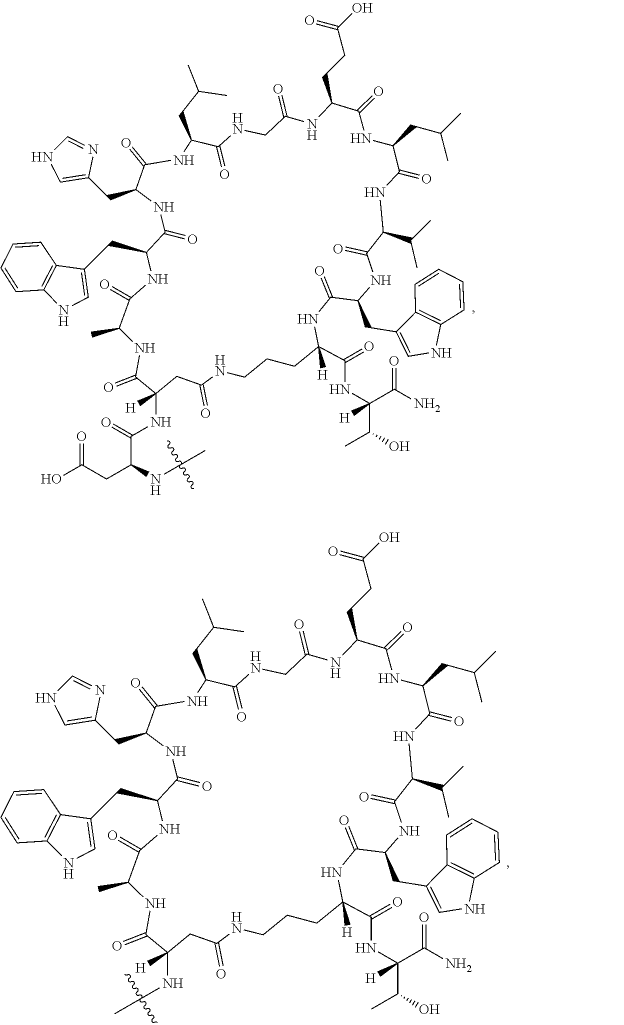

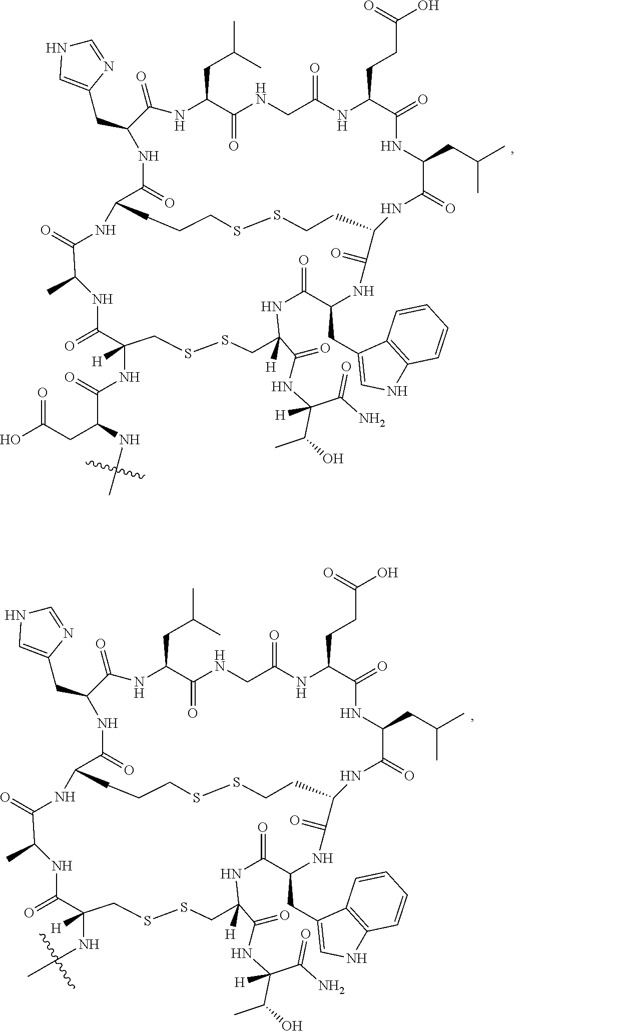 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2 ; *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2]

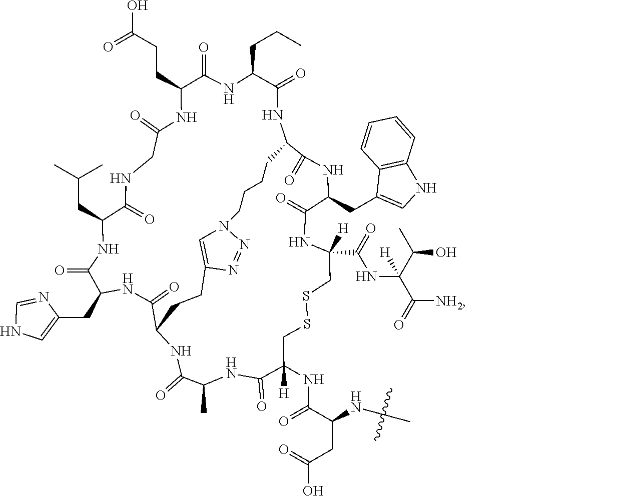 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2]

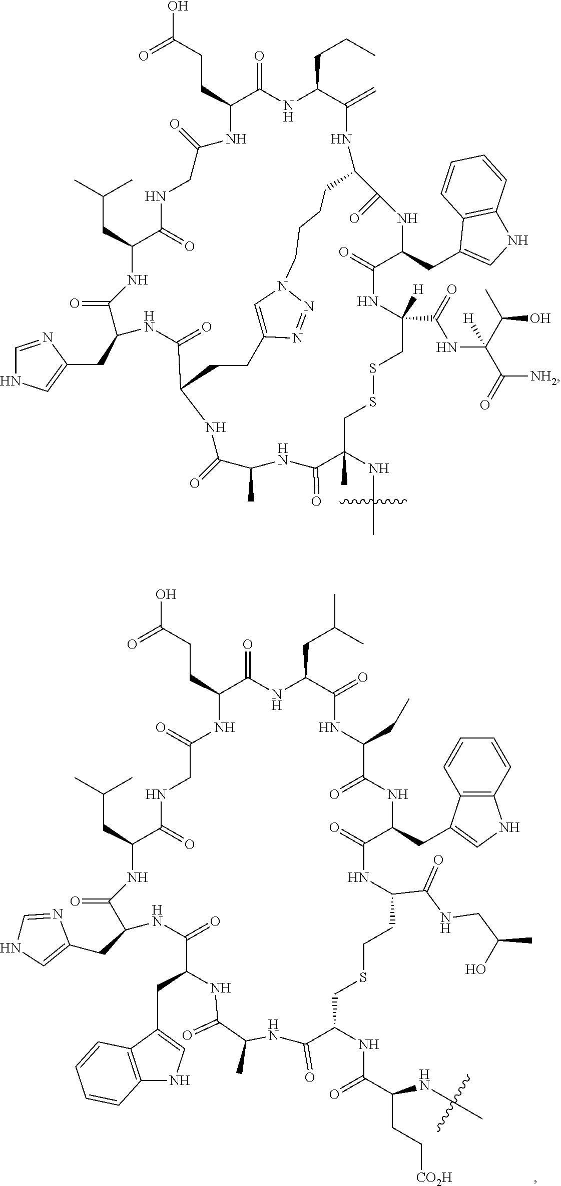 [SMILES: *N[C@@]1(C)CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=C)N2 ; *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

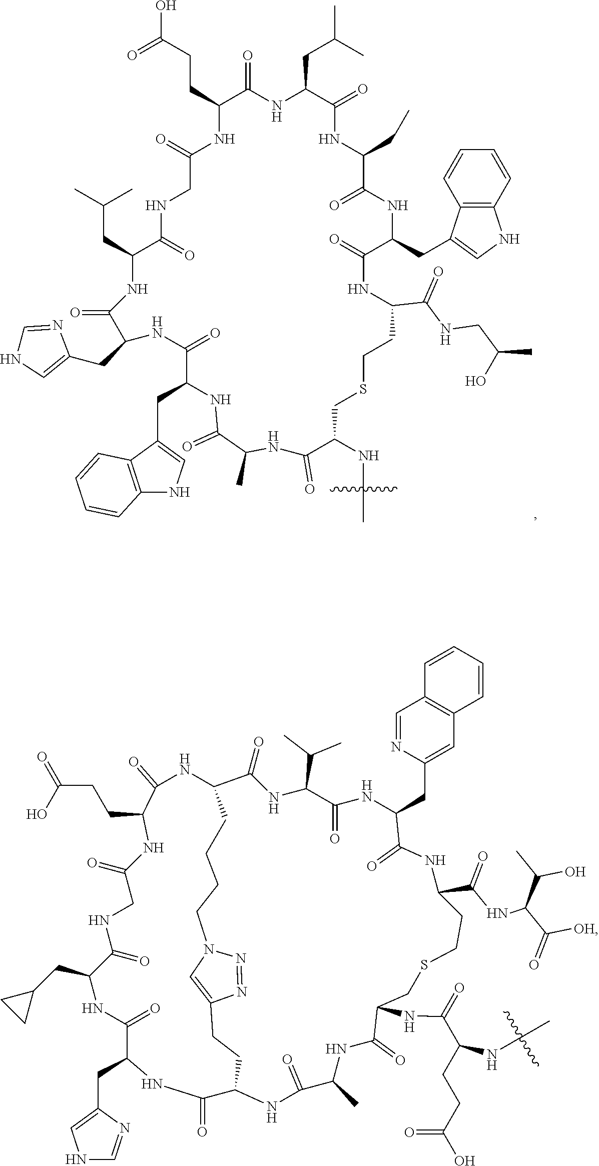 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2]

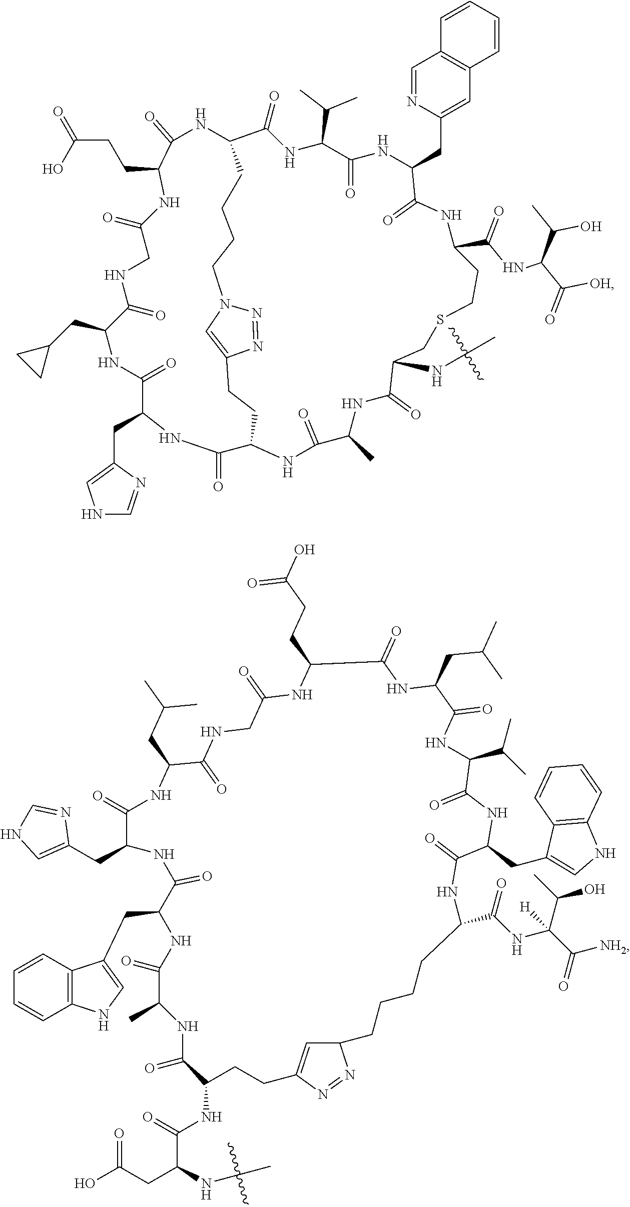

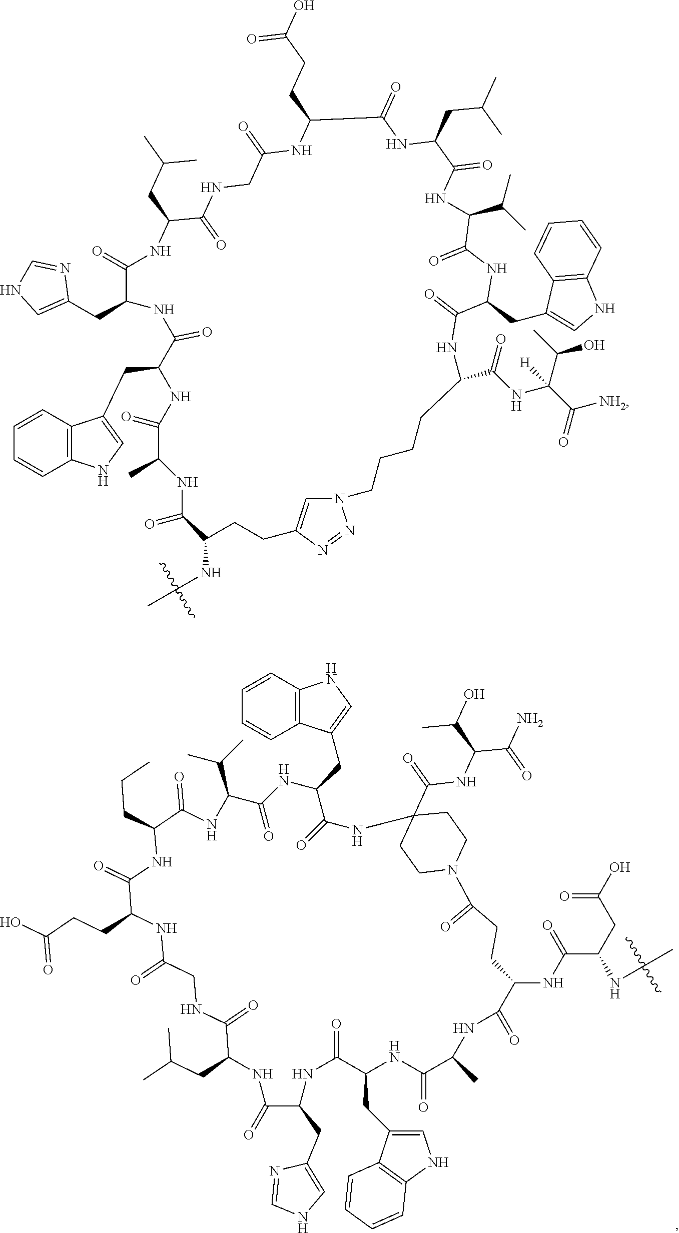

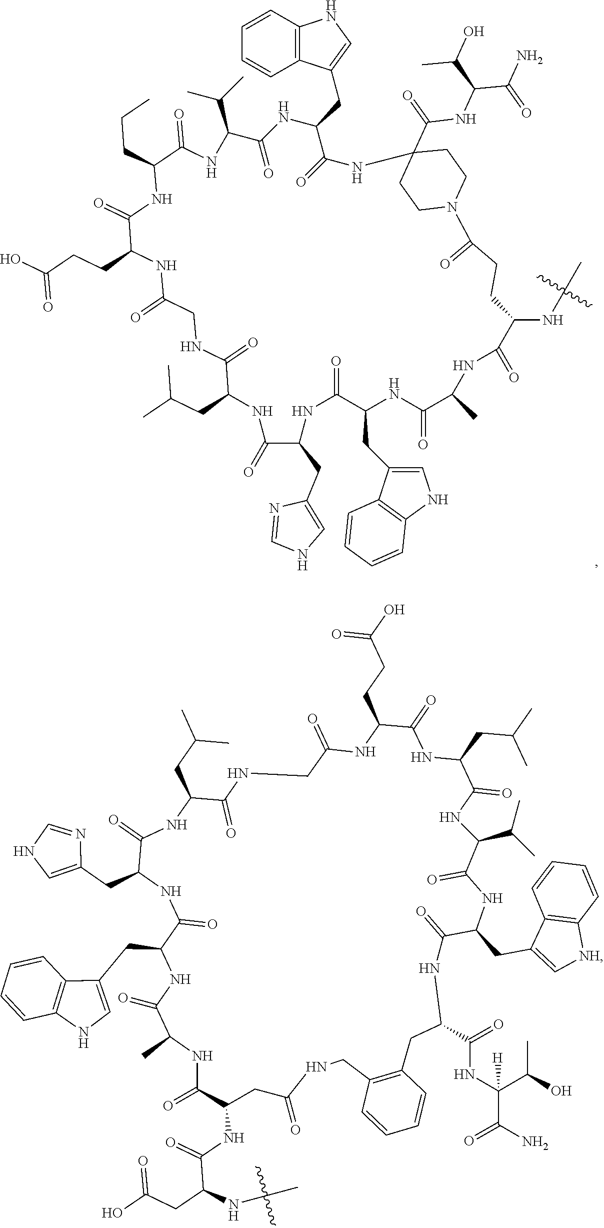

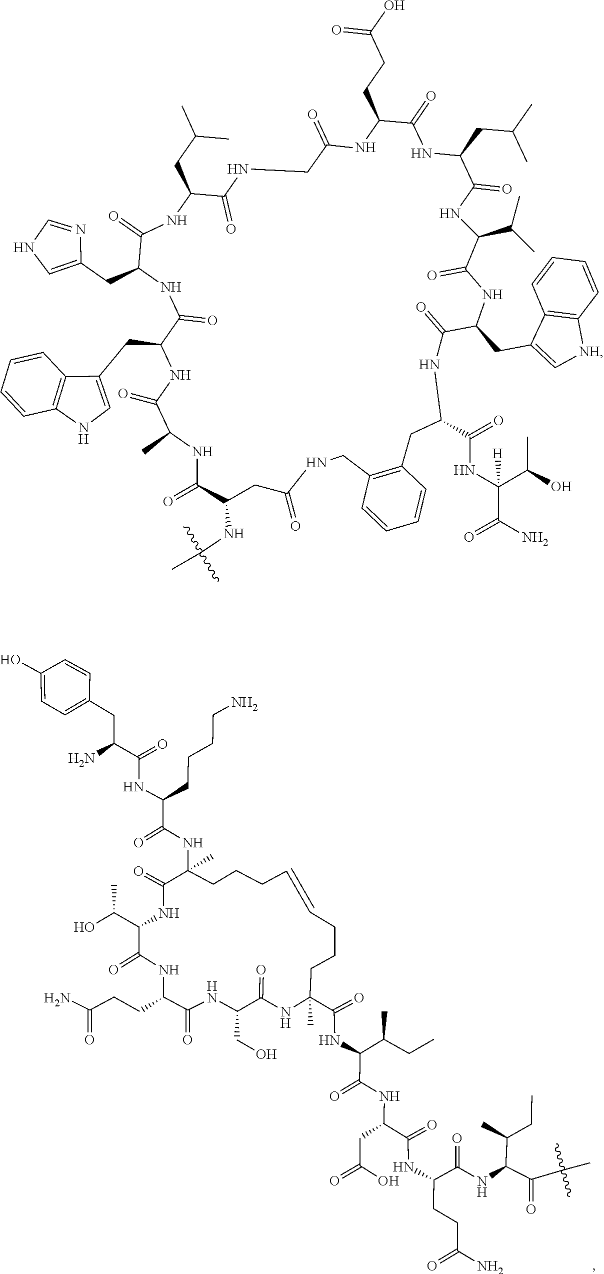

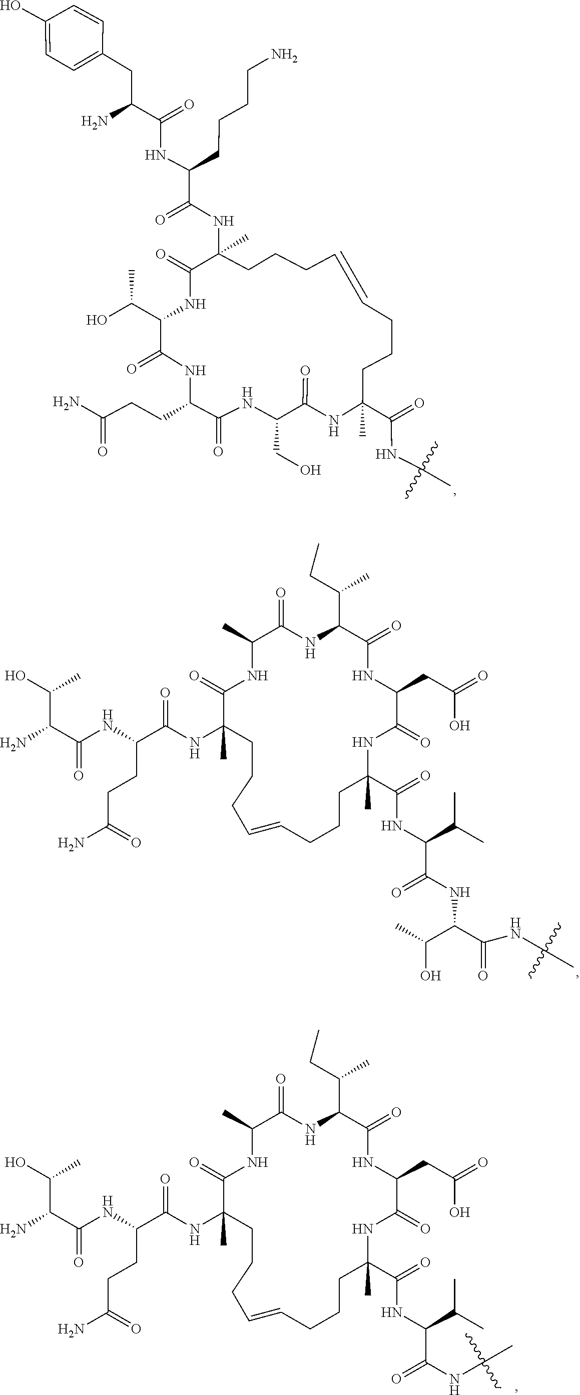

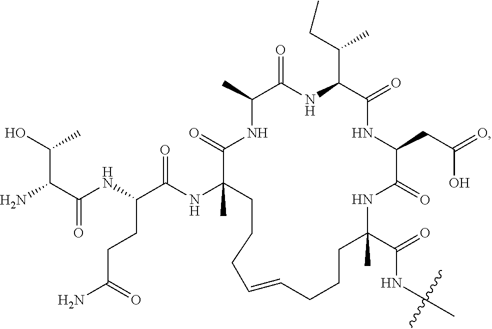 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1]

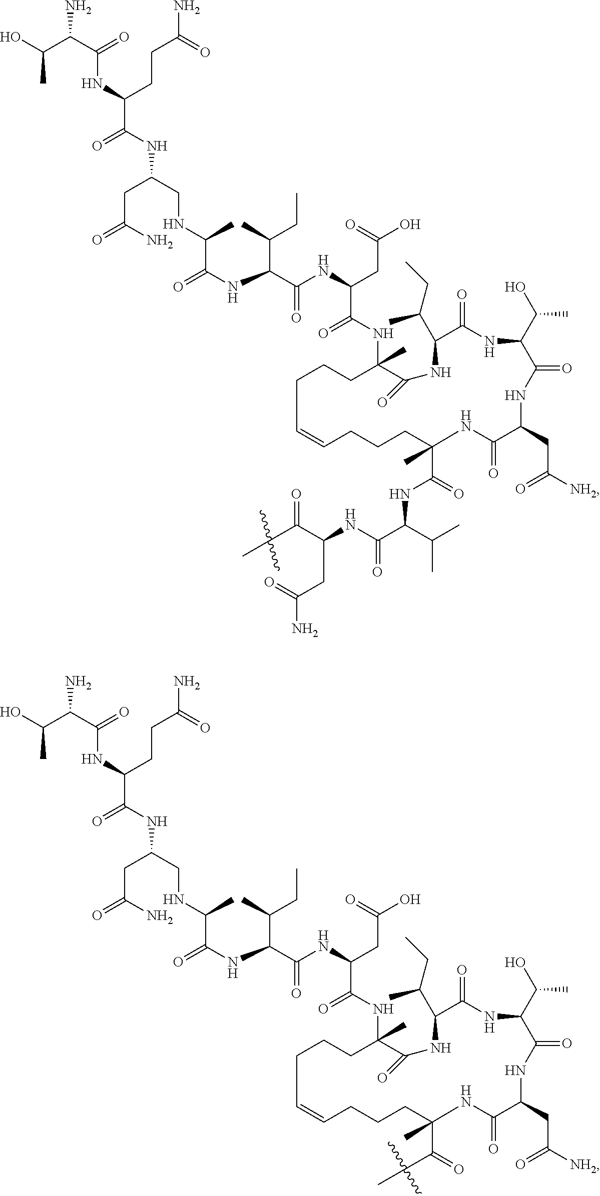

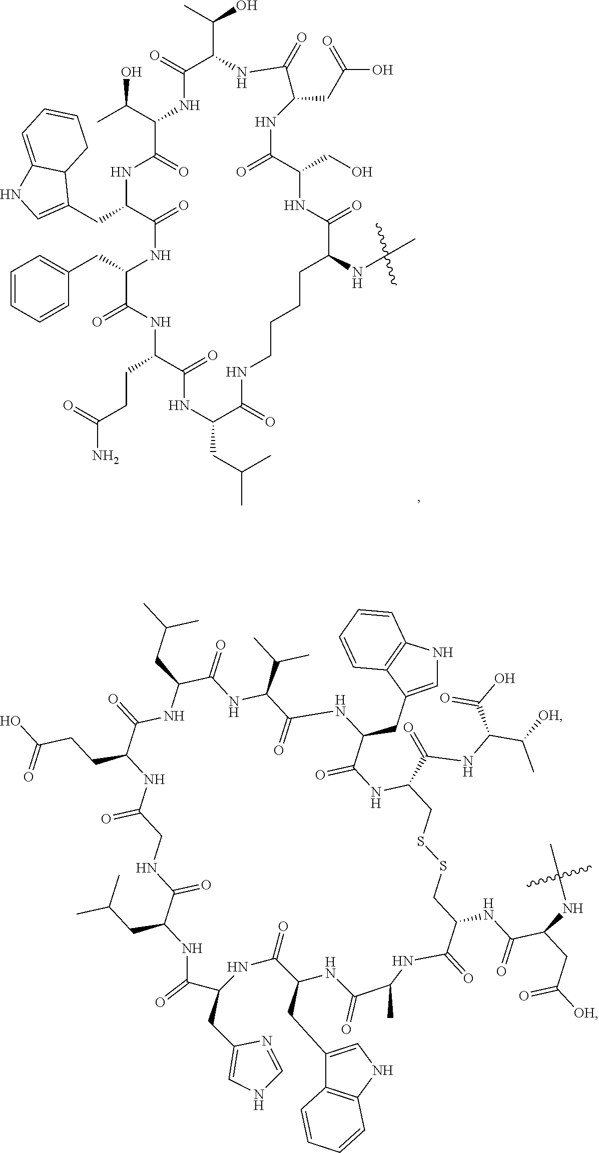 [SMILES: *N[C@H]1CCCCNC(=O)[C@H](CC(C)C)NC(=O)[C@H](CCC(N)=O)NC(=O)[C@H](Cc2ccccc2)NC(=O)[C@H](CC2=CNC3=CC=CCC23)NC(=O)[C@H]([C@@H](C)O)NC(=O)[C@H]([C@@H](C)O)NC(=O)[C@H](CC(=O)O)NC(=O)[C@H](CO)NC1=O ; *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

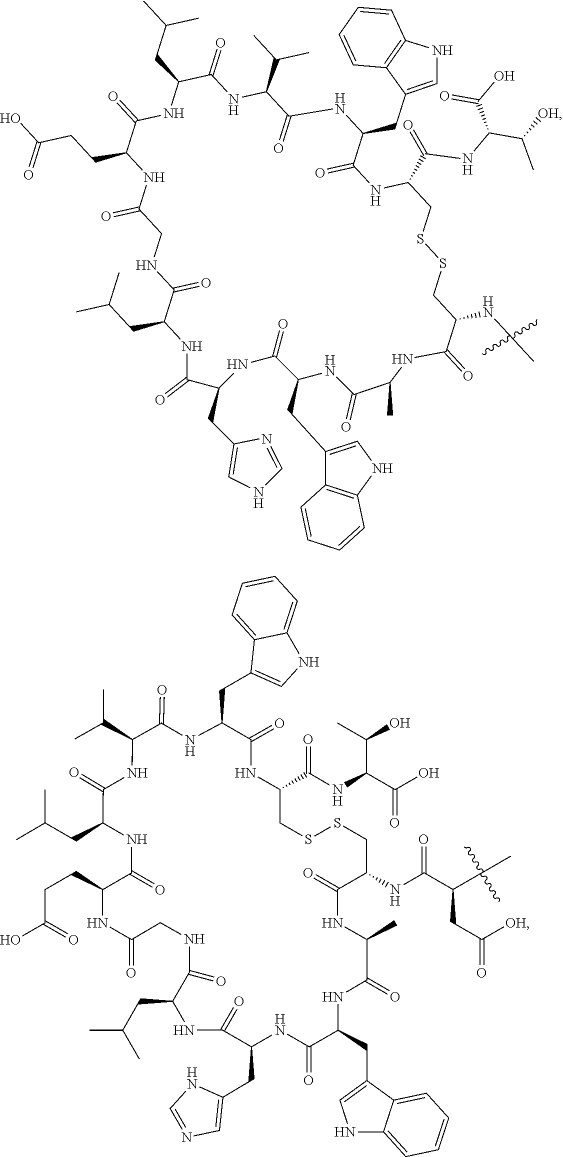 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O]

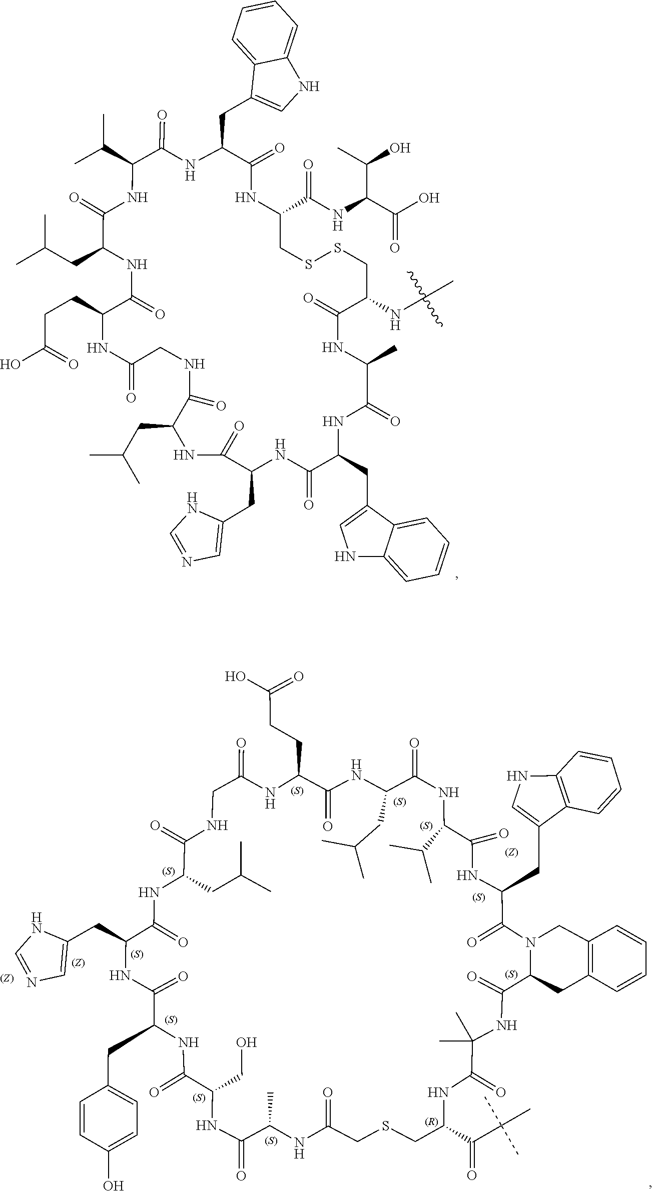

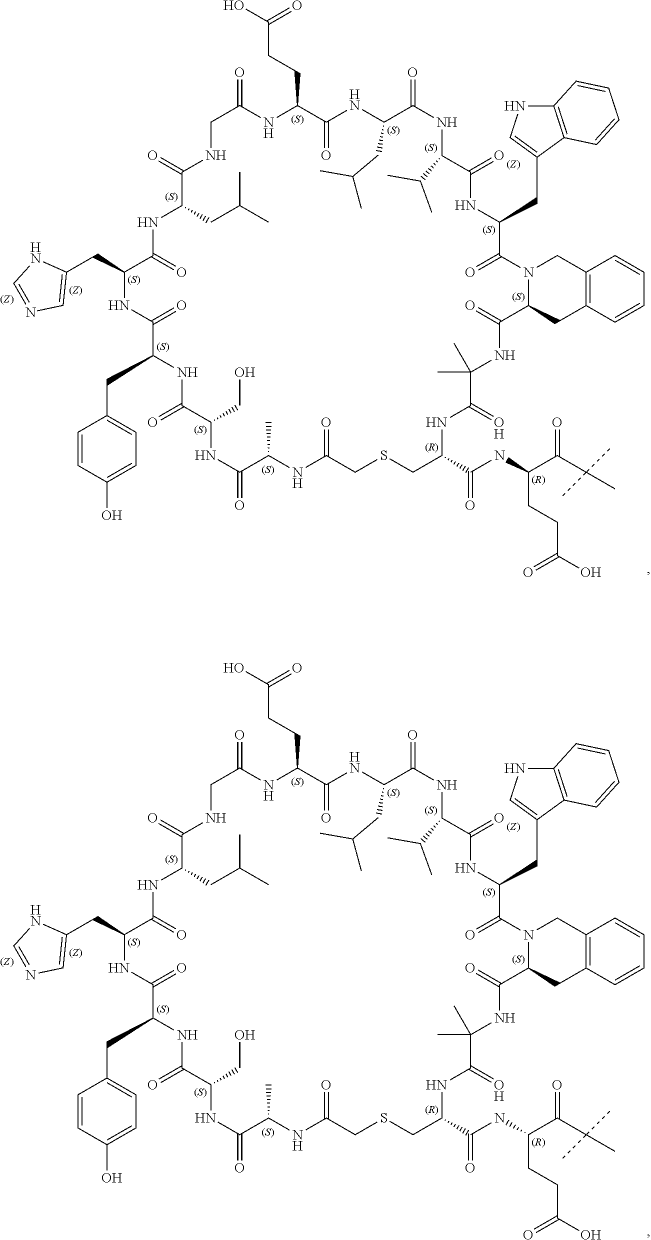

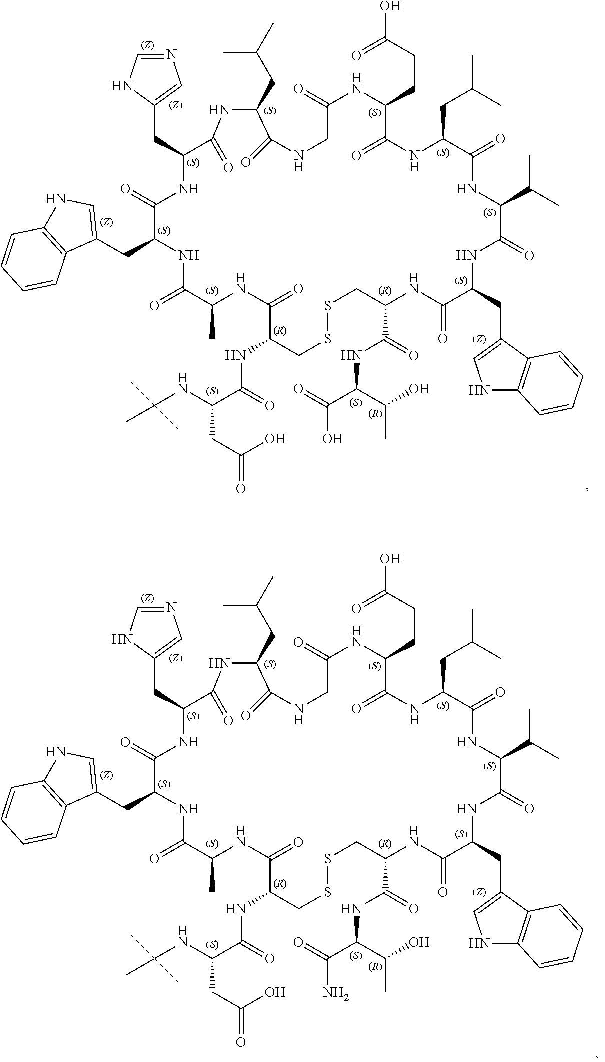

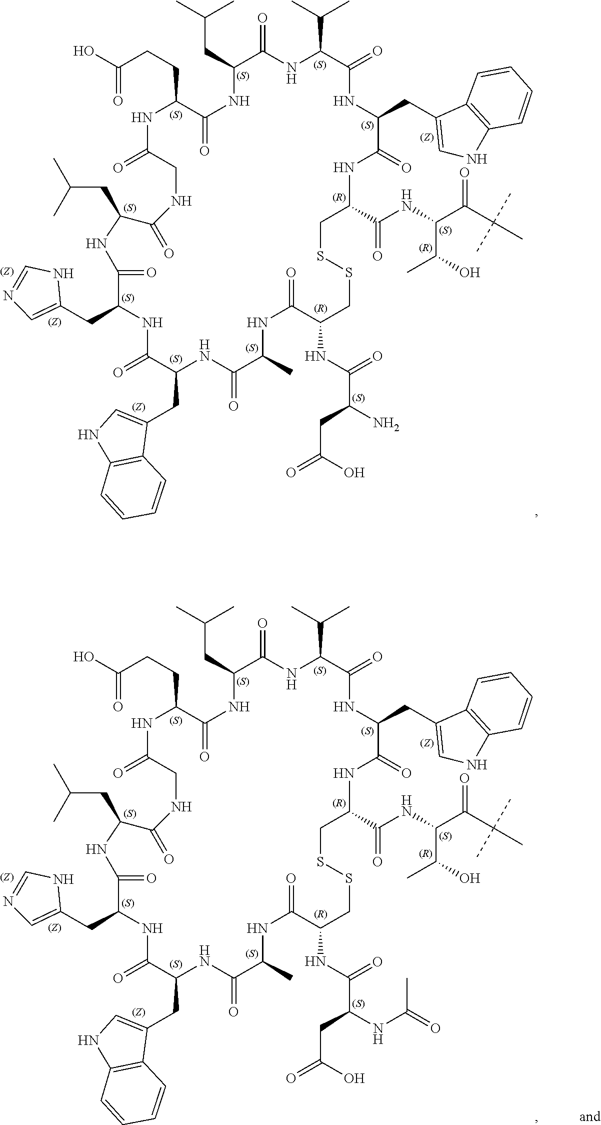

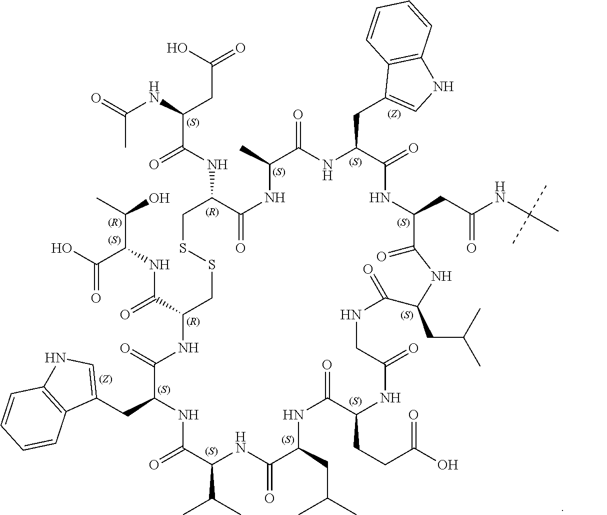

In any aspect or embodiment described herein, a universal antibody binding moiety comprises an ABM peptide unit, and is connected to a linker moiety (L) through the C-terminus of the ABM peptide unit. In any aspect or embodiment described herein, a universal antibody binding moiety comprises an ABM peptide unit, and is connected to a linker moiety (L) through the N-terminus of the ABM peptide unit. In any aspect or embodiment described herein, a universal antibody binding moiety comprises an ABM peptide unit, and is connected to a linker moiety (L) through a side chain group of the ABM peptide unit.

In any aspect or embodiment described herein, an antibody binding moiety (e.g., a universal antibody binding moiety) is or comprises a small molecule entity, with a molecular weight of, e.g., less than 10000, 9000, 8000, 7000, 6000, 5000, 4000, 3000, 2000, 1500, 1000, etc. In any aspect or embodiment described herein, the antibody binding moieties include small molecule Fc binder moieties, e.g., those described in U.S. Pat. No. 9,745,339, US 20130131321, etc.

As appreciated by those skilled in the art, in any aspect or embodiment described herein, antibodies of various properties and activities (e.g., antibodies recognizing different antigens, having optional modifications, etc.) may be recruited by antibody binding moieties described herein. In any aspect or embodiment described herein, such antibodies include antibodies administered to a subject, e.g., for therapeutic purposes. In any aspect or embodiment described herein, antibodies recruited by antibody binding moieties described herein comprise antibodies toward different antigens. In any aspect or embodiment described herein, antibodies recruited by antibody binding moieties described herein comprise antibodies whose antigens are not present on the surface or cell membrane of target cells (e.g., target cells, such as coronavirus infected cells, such as SARS-CoV-2 infected cells). In any aspect or embodiment described herein, antibodies recruited by antibody binding moieties described herein comprise antibodies target antigens present on the surface or cell membrane of targets (e.g., target cells, such as coronavirus infected cells). In any aspect or embodiment described herein, antigens on the surface of target cells may interfere with the structure, conformation, and/or one or more properties and/or activities of recruited antibodies that bind such antigens. In any aspect or embodiment described herein, the antibody binding moiety (e.g., universal antibody binding moiety) recruits antibodies of diverse specificities, and no more than about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% percent of recruited antibodies are directed toward the same antigen, protein, lipid, carbohydrate, etc. Among other things, one advantage of the compounds of the present disclosure is that having one or more universal antibody binding moiety can utilize diverse pools of antibodies, such as those present in serum. In any aspect or embodiment described herein, a universal antibody binding moiety as described herein (e.g., uABM utilized in an ARM) are contacted with a plurality of antibodies, wherein no more than about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% percent of the plurality of antibodies are directed toward the same antigen, protein, lipid, carbohydrate, etc.

### Exemplified Target Binding Moieties (TBM)

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
each of X^{T1} through X^{T59} is an independently selected amino acid residue (e.g., of an amino acid of formula NH(R^{a1})-L^{a1}-C(R^{a2})(R^{a3})-L^{a2}-COOH);
optionally two amino acid residues of the TBM are covalently linked (e.g., two Cys/S amino acid residues form a disulfide bond) to form a cyclic structure; and
optionally one or more of X^{T1}-X^{T10} and X^{T33}-X^{T59} are absent,

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
optionally two amino acid residues of the TBM are covalently linked (e.g., two Cys/S amino acid residues form a disulfide bond) to form a cyclic structure;
X_{T1} is absent or a hydrophobic amino acid (e.g., an amino acid residue that has a hydrophobic side chain), such as Ala/A, Aib, or an isosterically equivalent amino acid;
X_{T2} is absent or a polar uncharged amino acid (e.g., an amino acid residue with a polar uncharged side chain, such as at physiological pH about 7.4), such as Ser/S or an isosterically equivalent amino acid;
X^{T3} is
   absent,
   a negatively charged amino acid (e.g., an amino acid residue with a negatively charged side chain, such as at physiological pH about 7.4), such as Asp/D, D-Asp/dD, or an isosterically equivalent amino acid, or
   a hydrophobic amino acid, such as Ala/A, D-Ala/dA, or an isosterically equivalent amino acid;
X^{T4} is absent or a positively charged amino acid (e.g., an amino acid residue with a positively charged side chain, such as at physiological pH of about 7.4), such as Lys/K or an isosterically equivalent amino acid;
X^{T5} is absent or a negatively charges amino acid, such as Glu/E or an isosterically equivalent amino acid;
X^{T6} is absent or a hydrophobic amino acid, such as Trp/W or an isosterically equivalent amino acid;
X^{T7} is absent or a hydrophobic amino acid, such as Ile/I or an isosterically equivalent amino acid;
X^{T8} is absent or a hydrophobic amino acid, such as Leu/L or an isosterically equivalent amino acid;
X^{T9} is absent or a polar uncharged amino acid, such as Gln/Q or an isosterically equivalent amino acid;
X^{T10} is absent or a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or
   a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or a hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T38} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
X^{T42} is absent or a negatively charged amino acid, such as Asp/D or an isosterically equivalent amino acid;
X^{T4}3 is absent or a negatively charged amino acid, such as Glu/E or an isosterically equivalent amino acid;
X^{T44} is absent or a positively charged amino acid, such as R or an isosterically equivalent amino acid;
X^{T4}5 is absent or a hydrophobic amino acid, such as Leu/L or an isosterically equivalent amino acid;
X^{T46} is absent or a hydrophobic amino acid, such as Leu/L or an isosterically equivalent amino acid;
X^{T47} is absent or a negatively charged amino acid, such as Glu/E or an isosterically equivalent amino acid;
X^{T4}8 is absent or a negatively charged amino acid, such as Glu/E or an isosterically equivalent amino acid;
X^{T49} is absent or a hydrophobic amino acid, such as Ala/A or an isosterically equivalent amino acid;
X^{T10} is absent or a negatively charged amino acid, such as Glu/E or an isosterically equivalent amino acid;
X^{T11} is absent or a positively charged amino acid, such as Arg/R or an isosterically equivalent amino acid;
X^{T52} is absent or a hydrophobic amino acid, such as Leu/L or an isosterically equivalent amino acid;
X^{T53} is absent or a hydrophobic amino acid, such as Leu/L or an isosterically equivalent amino acid;
X^{T54} is absent or a negatively charged amino acid, such as Glu/E or an isosterically equivalent amino acid;
X^{T55} is absent or a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
   absent,
   a positively charged amino acid, such as Arg/R or an isosterically equivalent amino acid, or
   a hydrophobic amino acid, such as Ala/A, D-Ala/dA, or an isosterically equivalent amino acid;
X^{T59} is absent or a hydrophobic amino acid, such as 2-(methylamino)propanoic acid, Ala/A, or an isosterically equivalent amino acid;
optionally two amino acid residues of the TBM are covalently linked (e.g., two Cys/S amino acid residues form a disulfide bond) to form a cyclic structure; or
a combination thereof.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
optionally two amino acid residues of the TBM are covalently linked (e.g., two Cys/S amino acid residues form a disulfide bond) to form a cyclic structure,
X_{T1} is absent or a hydrophobic amino acid, such as Ala/A, Aib, or an isosterically equivalent amino acid;
X_{T2} is absent or a polar uncharged amino acid, such as Ser/S or an isosterically equivalent amino acid;
X^{T3} is
   a negatively charged amino acid, such as Asp/D, D-Asp/dD, or an isosterically equivalent amino acid, or
   a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X_{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X¹²³ is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or
   a Pro/P or an isosterically equivalent amino acid;
X^{T26} is a hydrophobic amino acid, such as Ala/A or an isosterically equivalent amino acid;
X^{T27} is
   a polar uncharged amino acid, such as Ser/S or an isosterically equivalent amino acid, or
   a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is
   a hydrophobic amino acid, such as Val/V or an isosterically equivalent amino acid, or
   a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or a hydrophobic amino acid, such as Leu/L or an isosterically equivalent amino acid;
X^{T34} is absent or a hydrophobic amino acid, such as Ile/I or an isosterically equivalent amino acid;
X^{T3}5 is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
X^{T42} is absent or a negatively charged amino acid, such as Asp/D or an isosterically equivalent amino acid;
X^{T43} is absent or a negatively charged amino acid, such as Glu/E or an isosterically equivalent amino acid;
X^{T44} is absent or a positively charged amino acid, such as R or an isosterically equivalent amino acid;
X^{T4}5 is absent or a hydrophobic amino acid, such as Leu/L or an isosterically equivalent amino acid;
X^{T46} is absent or a hydrophobic amino acid, such as Leu/L or an isosterically equivalent amino acid;
X^{T47} is absent or a negatively charged amino acid, such as Glu/E or an isosterically equivalent amino acid;
X^{T48} is absent or a negatively charged amino acid, such as Glu/E or an isosterically equivalent amino acid;
X^{T49} is absent or a hydrophobic amino acid, such as Ala/A or an isosterically equivalent amino acid;
X^{T50} is absent or a negatively charged amino acid, such as Glu/E or an isosterically equivalent amino acid;
X^{T51} is absent or a positively charged amino acid, such as Arg/R or an isosterically equivalent amino acid;
X^{T52} is absent or a hydrophobic amino acid, such as Leu/L or an isosterically equivalent amino acid;
X^{T53} is absent or a hydrophobic amino acid, such as Leu/L or an isosterically equivalent amino acid;
X^{T54} is absent or a negatively charged amino acid, such as Glu/E or an isosterically equivalent amino acid;
X^{T55} is absent or a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
   absent,
   a positively charged amino acid, such as Arg/R or an isosterically equivalent amino acid, or
   a hydrophobic amino acid, such as Ala/A, D-Ala/dA, or an isosterically equivalent amino acid;
X^{T59} is absent or a hydrophobic amino acid, such as 2-(methylamino)propanoic acid, Ala/A, or an isosterically equivalent amino acid; or
a combination thereof.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X³⁰X^{T31}X^{T32}X^{T33}X^{T34}X^{T35}X^{T36}X^{T37}X^{T38}X^{T39}X^{T40}X^{T41}, wherein:
optionally two amino acid residues of the TBM are covalently linked (e.g., two Cys/S amino acid residues form a disulfide bond) to form a cyclic structure,
X_{T1} is absent or a hydrophobic amino acid, such as Ala/A, Aib, or an isosterically equivalent amino acid;
X_{T2} is absent or a polar uncharged amino acid, such as Ser/S or an isosterically equivalent amino acid;
X^{T3} is
   a negatively charged amino acid, such as Asp/D, D-Asp/dD, or an isosterically equivalent amino acid, or
   a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T114} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T122} is
   an uncharged, non-polar amino acid, such as Gly/G or an isosterically equivalent amino acid, or
   a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or
   a Pro/P or an isosterically equivalent amino acid;
X^{T26} is a hydrophobic amino acid, such as Ala/A or an isosterically equivalent amino acid;
X^{T27} is
   a polar uncharged amino acid, such as Ser/S or an isosterically equivalent amino acid, or
   a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is
   a hydrophobic amino acid, such as Val/V or an isosterically equivalent amino acid, or
   a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or a hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid; or
a combination thereof

In any aspect or embodiment described herein, a compound or ARM, or a pharmaceutically acceptable salt thereof, one or more TBM or target binding moiety independently is or comprises: X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
optionally two amino acid residues of the TBM are covalently linked (e.g., two Cys/S amino acid residues form a disulfide bond) to form a cyclic structure,
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is an uncharged, non-polar amino acid, such as Gly/G or an isosterically equivalent amino acid, or
   a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or
   a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is
   a hydrophobic amino acid, such as Val/V or an isosterically equivalent amino acid, or
   a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
X^{T42} is absent or a negatively charged amino acid, such as Asp/D or an isosterically equivalent amino acid;
X^{T43} is absent or a negatively charged amino acid, such as Glu/E or an isosterically equivalent amino acid;
X^{T44} is absent or a positively charged amino acid, such as R or an isosterically equivalent amino acid;
X^{T45} is absent or a hydrophobic amino acid, such as Leu/L or an isosterically equivalent amino acid;
X^{T46} is absent or a hydrophobic amino acid, such as Leu/L or an isosterically equivalent amino acid;
X^{T47} is absent or a negatively charged amino acid, such as Glu/E or an isosterically equivalent amino acid;
X^{T48} is absent or a negatively charged amino acid, such as Glu/E or an isosterically equivalent amino acid;
X^{T49} is absent or a hydrophobic amino acid, such as Ala/A or an isosterically equivalent amino acid;
X^{T50} is absent or a negatively charged amino acid, such as Glu/E or an isosterically equivalent amino acid;
X^{T51} is absent or a positively charged amino acid, such as Arg/R or an isosterically equivalent amino acid;
X^{T52} is absent or a hydrophobic amino acid, such as Leu/L or an isosterically equivalent amino acid;
X^{T53} is absent or a hydrophobic amino acid, such as Leu/L or an isosterically equivalent amino acid;
X^{T54} is absent or a negatively charged amino acid, such as Glu/E or an isosterically equivalent amino acid;
X^{T55} is absent or a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
   absent,
   a positively charged amino acid, such as Arg/R or an isosterically equivalent amino acid, or
   a hydrophobic amino acid, such as Ala/A, D-Ala/dA, or an isosterically equivalent amino acid;
X^{T59} is absent or a hydrophobic amino acid, such as 2-(methylamino)propanoic acid, Ala/A, or an isosterically equivalent amino acid; or
a combination thereof.

In any aspect or embodiment described herein, a compound or ARM, or a pharmaceutically acceptable salt thereof, one or more TBM or target binding moiety independently is or comprises: X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}, wherein:
optionally two amino acid residues of the TBM are covalently linked (e.g., two Cys/S amino acid residues form a disulfide bond) to form a cyclic structure,
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X¹²³ is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or
   a Pro/P or an isosterically equivalent amino acid;
X^{T26} is a hydrophobic amino acid, such as Ala/A or an isosterically equivalent amino acid;
X^{T27} is
   a polar uncharged amino acid, such as Ser/S or an isosterically equivalent amino acid, or
   a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is
   a hydrophobic amino acid, such as Val/V or an isosterically equivalent amino acid, or
   a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T3}8 is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid; or
a combination thereof.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}, wherein:
X_{T1} is absent or a hydrophobic amino acid, such as Ala/A, Aib, or an isosterically equivalent amino acid;
X_{T2} is absent or a polar uncharged amino acid, such as Ser/S or an isosterically equivalent amino acid;
X^{T3} is
   a negatively charged amino acid, such as Asp/D, D-Asp/dD, or an isosterically equivalent amino acid, or
   a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T114} is a hydrophobic amino acid, such as Ile/I or an isosterically equivalent amino acid;
X^{T15} is a hydrophobic amino acid, such as Met/M, norleucine/Nle, or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or a Pro/P or an isosterically equivalent amino acid;
X^{T26} is a hydrophobic amino acid, such as Ala/A or an isosterically equivalent amino acid;
X^{T27} is
   a polar uncharged amino acid, such as Ser/S or an isosterically equivalent amino acid, or
   a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
optionally X^{T28} and X^{T27} form a disulfide bond; or
a combination thereof.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41} wherein:
X_{T1} is absent or a hydrophobic amino acid, such as Ala/A, Aib, or an isosterically equivalent amino acid;
X_{T2} is absent or a polar uncharged amino acid, such as Ser/S or an isosterically equivalent amino acid;
X^{T3} is
   a negatively charged amino acid, such as Asp/D, D-Asp/dD, or an isosterically equivalent amino acid, or
   a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X¹ is a positively charged amino acid, such as His/H or an isosterically equivalent amino acid;
X^{T24} is a hydrophobic amino acid, such as Ala/A or an isosterically equivalent amino acid;
X^{T25} is
   a negatively charged amino acid, such as Glu/E or an isosterically equivalent amino acid, or a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is
   a hydrophobic amino acid, such as Val/V or an isosterically equivalent amino acid, or
   a Cys/C or an isosterically equivalent amino acid;
X^{T31} is
   a polar uncharged amino acid, such as Ser/S or an isosterically equivalent amino acid, or
   a negatively charged amino acid, such as Glu/E or an isosterically equivalent amino acid;
X^{T32} is a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid;
X^{T37} is absent or a hydrophobic amino acid, such as Phe/F or an isosterically equivalent amino acid;
X^{T38} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T4}° is
   absent,
   a positively charged amino acid, such as Lys/K or an isosterically equivalent amino acid, or
   a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
optionally X^{T15} and X^{T30} form a disulfide bond; or
a combination thereof.

In any aspect or embodiment described herein, a compound or ARM, or a pharmaceutically acceptable salt thereof, one or more TBM or target binding moiety independently is or comprises: X^{T1a}X^{T2a}X^{T3a}X^{T4a}X^{T5a}X^{T6a}X^{T7a}X^{T8a}X^{T9a}X^{T10a}X^{T11a}X^{T12a}X^{T13a}X^{T14a}X^{T15a}X^{T16a}X^{T17a}X^{T18a}X^{T19a}X^{T20a}X^{T21a}X^{T22a}X^{T23a}X^{T24a}X^{T25a}X^{T26a}X^{T27a}X^{T28a}X^{T29a}X^{T30a}X^{T31a}X^{T32a}X^{T33a}X^{T34a}X^{T35a}X^{T36a}X^{T37a}X^{T38a}X^{T39a}X^{T40a}X^{T41a}X^{T42a}X^{T43a}X^{T44a}X^{T45a}X^{T46a}X^{T47a}X^{T48a}X^{T49a}X^{T50a}X^{T51a}X^{T52a}X^{T53a}X^{T54a}X^{T55a}X^{T56a}X^{T57a}X^{T58a}X^{T59a}X^{T60a}X^{T61a}X^{T62a}X^{T63a}X^{T64a}, wherein each of X^{T1a} through X^{T64a} is an independently selected amino acid residue (e.g., of an amino acid of formula NH(R^{a1})-L^{a1}-C(R^{a2})(R^{a3})-L^{a2}-COOH).

In any aspect or embodiment described herein, a compound or ARM, or a pharmaceutically acceptable salt thereof, one or more TBM or target binding moiety independently is or comprises: X^{T1a}X^{T2a}X^{T3a}X^{T4a}X^{T5a}X^{T6a}X^{T7a}X^{T8a}X^{T9a}X^{T10a}X^{T11a}X^{T12a}X^{T13a}X^{T14a}X^{T15a}X^{T16a}X^{T17a}X^{T18a}X^{T19a}X^{T20a}X^{T21a}X^{T22a}X^{T23a}X^{T24a}X^{T25a}X^{T26a}X^{T27a}X^{T28a}X^{T29a}X^{T30a}X^{T31a}X^{T32a}X^{T33a}X^{T34a}X^{T35a}X^{T36a}X^{T37a}X^{T38a}X^{T39a}X^{T40a}X^{T41a}X^{T42a}X^{T43a}X^{T44a}X^{T45a}X^{T46a}X^{T47a}X^{T48a}X^{T49a}X^{T50a}X^{T51a}X^{T52a}X^{T53a}X^{T54a}X^{T55a}X^{T56a}X^{T57a}X^{T58a}X^{T59a}X^{T60a}X^{T61a}X^{T62a}X^{T63a}X^{T64a} wherein:
X^{T1a} is an uncharged, polar amino acid, such as Asn/N or an isosterically equivalent amino acid;
X^{T2a} is a negatively charged amino acid, such as Asp/D or an isosterically equivalent amino acid;
X^{T3a} is a negatively charged amino acid, such as Asp/D or an isosterically equivalent amino acid;
X^{T4a} is a negatively charged amino acid, such as Glu/E or an isosterically equivalent amino acid;
X^{T5a} is a hydrophobic amino acid, such as Leu/L or an isosterically equivalent amino acid;
X^{T6a} is a positively charged amino acid, such as His/H or an isosterically equivalent amino acid;
X^{T7a} is a hydrophobic amino acid, such as Met/M or an isosterically equivalent amino acid;
X^{T8a} is a hydrophobic amino acid, such as Leu/L or an isosterically equivalent amino acid;
X^{T9a} is a hydrophobic amino acid, such as Met/M or an isosterically equivalent amino acid;
X^{T10a} is a hydrophobic amino acid, such as Tyr/T or an isosterically equivalent amino acid;
X^{T11a} is a negatively charged amino acid, such as Asp/D or an isosterically equivalent amino acid;
X^{T12a} is a hydrophobic amino acid, such as Leu/L or an isosterically equivalent amino acid;
X^{T13a} is a hydrophobic amino acid, such as Val/V or an isosterically equivalent amino acid;
X^{T14a} is a hydrophobic amino acid, such as Tyr/Y or an isosterically equivalent amino acid;
X^{T15a} is a negatively charged amino acid, such as Glu/E or an isosterically equivalent amino acid;
X^{T16a} is a hydrophobic amino acid, such as Ala/A or an isosterically equivalent amino acid;
X^{T17a} is a hydrophobic amino acid, such as Leu/L or an isosterically equivalent amino acid;
X^{T18a} is a positively charged amino acid, such as His/H or an isosterically equivalent amino acid;
X^{T19a} is a hydrophobic amino acid, such as Phe/F or an isosterically equivalent amino acid;
X^{T27a} is a hydrophobic amino acid, such as Ala/A or an isosterically equivalent amino acid;
X^{T21a} is a positively charged amino acid, such as Lys/K or an isosterically equivalent amino acid;
X^{T22a} is a negatively charged amino acid, such as Asp/D or an isosterically equivalent amino acid;
X^{T23a} is a negatively charged amino acid, such as Glu/E or an isosterically equivalent amino acid;
X^{T24a} is a negatively charged amino acid, such as Glu/E or an isosterically equivalent amino acid;
X^{T25a} is a hydrophobic amino acid, such as Ile/I or an isosterically equivalent amino acid;
X^{T26a} is a positively charged amino acid, such as Lys/K or an isosterically equivalent amino acid;
X^{T27a} is a positively charged amino acid, such as Lys/K or an isosterically equivalent amino acid;
X^{T28a} is a positively charged amino acid, such as Arg/R or an isosterically equivalent amino acid;
X^{T29a} is a hydrophobic amino acid, such as Val/V or an isosterically equivalent amino acid;
X^{T30a} is a hydrophobic amino acid, such as Phe/F or an isosterically equivalent amino acid;
X^{T31a} is an uncharged, polar amino acid, such as Gln/Q or an isosterically equivalent amino acid;
X^{T32a} is a hydrophobic amino acid, such as Leu/L or an isosterically equivalent amino acid;
X^{T33a} is a hydrophobic amino acid, such as Phe/F or an isosterically equivalent amino acid;
X^{T34a} is a negatively charged amino acid, such as Glu/E or an isosterically equivalent amino acid;
X^{T35a} is a hydrophobic amino acid, such as Leu/L or an isosterically equivalent amino acid;
X^{T16a} is a hydrophobic amino acid, such as Ala/A or an isosterically equivalent amino acid;
X^{T17a} is a negatively charged amino acid, such as Asp/D or an isosterically equivalent amino acid;
X^{T23a} is a positively charged amino acid, such as Lys/K or an isosterically equivalent amino acid;
X^{19a} is a hydrophobic amino acid, such as Ala/A or an isosterically equivalent amino acid;
X^{T40a} is a hydrophobic amino acid, such as Tyr/Y or an isosterically equivalent amino acid;
X^{T41a} is a positively charged amino acid, such as Lys/K or an isosterically equivalent amino acid;
X^{T42a} is an uncharged, polar amino acid, such as Asn/N or an isosterically equivalent amino acid;
X^{T43a} is an uncharged, polar amino acid, such as Asn/N or an isosterically equivalent amino acid;
X^{T44a} is a negatively charged amino acid, such as Asp/D or an isosterically equivalent amino acid;
X^{T45a} is a positively charged amino acid, such as Arg/R or an isosterically equivalent amino acid;
X^{T46a} is an uncharged, polar amino acid, such as Gln/Q or an isosterically equivalent amino acid;
X^{T47a} is a positively charged amino acid, such as Lys/K or an isosterically equivalent amino acid;
X^{T48a} is a hydrophobic amino acid, such as Leu/L or an isosterically equivalent amino acid;
X^{T49a} is a negatively charged amino acid, such as Glu/E or an isosterically equivalent amino acid;
X^{T50a} is a positively charged amino acid, such as Lys/K or an isosterically equivalent amino acid;
X^{T51a} is a hydrophobic amino acid, such as Val/V or an isosterically equivalent amino acid;
X^{T52a} is a hydrophobic amino acid, such as Val/V or an isosterically equivalent amino acid;
X^{T53a} is a negatively charged amino acid, such as Glu/E or an isosterically equivalent amino acid;
X^{T54a} is a negatively charged amino acid, such as Glu/E or an isosterically equivalent amino acid;
X^{T55a} is a hydrophobic amino acid, such as Leu/L or an isosterically equivalent amino acid;
X^{T56a} is a positively charged amino acid, such as Lys/K or an isosterically equivalent amino acid;
X^{T57a} is a negatively charged amino acid, such as Glu/E or an isosterically equivalent amino acid;
X^{T58a} is a hydrophobic amino acid, such as Leu/L or an isosterically equivalent amino acid;
X^{T59a} is a hydrophobic amino acid, such as Leu/L or an isosterically equivalent amino acid;
X^{T60a} is a negatively charged amino acid, such as Glu/E or an isosterically equivalent amino acid;
X^{T61a} is a positively charged amino acid, such as Arg/R or an isosterically equivalent amino acid;
X^{T62a} is a hydrophobic amino acid, such as Leu/L or an isosterically equivalent amino acid;
X^{T63a} is a hydrophobic amino acid, such as Leu/L or an isosterically equivalent amino acid;
X^{T64a} is an uncharged, polar amino acid, such as Ser/S or an isosterically equivalent amino acid; or
a combination thereof.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}, wherein:
each of X^{T1} through X^{T36} is an independently selected amino acid residue (e.g., of an amino acid of formula NH(R^{a1})-L^{a1}-C(R^{a2})(R^{a3})-L^{a2}-COOH);
optionally two amino acid residues of the TBM are covalently linked (e.g., two Cys/S amino acid residues form a disulfide bond) to form a cyclic structure; and optionally one or more of X^{T1}, X^{T2}, and X^{T23}-X^{T36} are absent.

In any aspect or embodiment described herein, a compound or ARM, or a pharmaceutically acceptable salt thereof, one or more TBM or target binding moiety independently is or comprises: X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36} wherein:
optionally two amino acid residues of the TBM are covalently linked (e.g., two Cys/S amino acid residues form a disulfide bond) to form a cyclic structure; X_{T1} is absent or a hydrophobic amino acid, such as Ala/A, Aib, or an isosterically equivalent amino acid; X_{T2} is absent or a polar uncharged amino acid, such as Ser/S or an isosterically equivalent amino acid;
X^{T3} is
   absent,
   a negatively charged amino acid, such as Asp/D, D-Asp/dD, or an isosterically equivalent amino acid, or
   a hydrophobic amino acid, such as Ala/A, D-Ala/dA, or an isosterically equivalent amino acid;
X^{T4} is absent or a positively charged amino acid, such as Lys/K or an isosterically equivalent amino acid;
X^{T5} is absent or a negatively charges amino acid, such as Glu/E or an isosterically equivalent amino acid;
X^{T6} is absent or hydrophobic amino acid, such as Trp/W or an isosterically equivalent amino acid;
X^{T7} is absent or a hydrophobic amino acid, such as Ile/I or an isosterically equivalent amino acid;
X^{T8} is absent or a hydrophobic amino acid, such as Leu/L or an isosterically equivalent amino acid;
X^{T9} is absent or a polar uncharged amino acid, such as Gln/Q or an isosterically equivalent amino acid;
X^{T10} is absent or a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is
   a hydrophobic amino acid, such as Met/M, norleucine/Nle, or an isosterically equivalent amino acid, or
   a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is
   a hydrophobic amino acid, such as Leu/L or an isosterically equivalent amino acid, or
   a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
   an uncharged, non-polar amino acid, such as Gly/G or an isosterically equivalent amino acid, or
   a positively charged amino acid, such as His/H, D-His/dH, or an isosterically equivalent amino acid;
X^{T23} is absent or positively charged amino acid, such as His/H or an isosterically equivalent amino acid;
X^{T24} is absent or a hydrophobic amino acid, such as Ala/A or an isosterically equivalent amino acid;
X^{T25} is
   absent,
   a negatively charged amino acid, such as Glu/E or an isosterically equivalent amino acid, or a Pro/P or an isosterically equivalent amino acid;
X^{T26} is absent or a hydrophobic amino acid, such as Ala/A or an isosterically equivalent amino acid;
X^{T27} is
   absent,
   a polar uncharged amino acid, such as Ser/S or an isosterically equivalent amino acid, or
   a Cys/C or an isosterically equivalent amino acid;
X^{T28} is absent or a hydrophobic amino acid, such as Met/M, norleucine/Nle, or an isosterically equivalent amino acid;
X^{T29} is absent or a positively charged amino acid, such as Arg/R or an isosterically equivalent amino acid;
X^{T30} is
   absent,
   a hydrophobic amino acid, such as Val/V or an isosterically equivalent amino acid, or
   a Cys/C or an isosterically equivalent amino acid;
X^{T31} is
   absent,
   a polar uncharged amino acid, such as Ser/S or an isosterically equivalent amino acid, or
   a negatively charged amino acid, such as Glu/E or an isosterically equivalent amino acid;
X^{T132} is absent or a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
   absent,
   a negatively charged amino acid, such as Glu/E or an isosterically equivalent amino acid, or
   a hydrophobic amino acid, such as Aib, Ala/A, or an isosterically equivalent amino acid; or
a combination thereof.

In any aspect or embodiment described herein, one or more TBM, independently, is or comprises a TBM from an exemplary compound of the present disclosure.

In any aspect or embodiment described herein, one or more TBM, independently, is or comprises an optionally substituted amino acid sequence selected from:

DKEWILQKIYEIMRLLDELGHAEASMRVSDLIYEFMKKGDERLLEEAERL
LEEVER,
DCAWILQKIYEIMRLLDELGHAEASMRVSDLIYEFMKKGDERLLEEAERL
LEEVER,
DKEWILQKIYEIMRLLDELGHAPASMRVSDLIYEFMKKGDERLLEEAERL
LEEVER,
DKEWILQKIYEIMRLLDELGHAEASMRVSDLIYEFMKKG,
DKEWILQKIYEIMRLLDELGHAEASMRVSDLIYEFMKK,
DKEWILQKIYEIMRLLDELGHAEASMRVSDLIYE,
DKEWILQKIYEIMRLLDELGHAPASMRVER,
DKEWILQKIYEIMRLLDELGHAEASMRVS,
DKEWILQKIYEIMRLLDELGHAEAS,
DKEWILQKIYEIMRLLDELG,
ILQKIYEIMRLLDELGHAEASMRVSDLIYEFMKKG,
IYEIMRLLDELGHAEASMRVSDLIYEFMKKG,
ILQKIYEIMRLLDELGHAEASMRVSDLIYEFMKK,
[Aib]DKEWILQKIYEIMRLLDELGHAEASMRVSDLIYEFMKKG,
[Aib]SDKEWILQKIYEIMRLLDELGHAEASMRVSDLIYEFMKK[dA],
[Aib]SDKEWILQKIYEIMRLLDELGHAEASMRVSDLIYEFMKK[dF],
D[Abu]EWILQKIYEIMRLLDELG,
[Aib]SDKEWILQKIYEI[Nle]RLLDELGHAE,
[Aib]SDKEWILQLIYEIMRLLDELGHAEASMRVSDLIYEFMKK[dA],
[Aib]SDKEWILQKIYEIMRLLDELGHAEASMRVSDLIYEFMKK[F],
[Aib]SDKEWILQKIYEIMRLLDELGHAEAS[Nle]RVSDLIY[Aib]FM
K[Aib]G,
DKEWILQKIYEIMRLLDEL[DHis]HAPASMRVSDLIYEFMKKGDERLLE
EAERLLEEVER,
DKEWILQLIYEI[Nle]RLLDELGHAEASMRVSDLIYEF[Nle]KKGDER
LLEEAERLLEEVER,
DKEWILQKIYEIMRLLDE[Nle][Dhis]HAEASMRVSDLIYEFMKKGDE
RLLEEAERLLEEVER,
DKEWILQKIYEIMRLLDE[Nle][Dhis]HAEASMRVSDLIYEFMKKG,
DKEWILQKIYE[d]]MRLLDELGHAEASMRVSDLIYEFMKKGDERLLEEA
ERLLEEVER,
DKEWILQKIYEIMRLLDEL[Dhis]HAPASMRVSDLIYEFMKKGDERLLE
EAERLLEEVER,
[Aib]SDKEWILQKIYEI[Nle]RLLDELGHAEASM
RVSDLIYEHMKKGDERLLEEAERLLE[Aib]VER[Aib],
[Aib]SDKEWILQKIYEIMRLLDELGHAEAS[Nle]RVSDLIY[Aib]FM
K[Aib]G,
[Aib]SDKEWILQKIYEI[Nle]RLLDELGHAEAS[Nle]RVSDLIY
[Aib]FMK[Aib]G,
SDKEWILQKIYEIMRLLDELGHAPASMRVSDLIYEFMKKG,
DKEWILQKIYEIMRLLDELGHAEASMRVSDLIYEFMKKGDERLLEEAERL
LEEVE[DAla],
DKEWILQKIYEIMRLLDELGHAPASMRVSDLIYEFMKKGDERLLEEAERL
LEEVE[DAla],
DKEWILQKIYEIMRLLDELGHAEASMRVSDLIYEFMKKGDERLLEEAERL
LEEVEA[2-
(methylamino)propanoic acid],
[Dala]KEWILQKIYEIMRLLDELGHAPASMRVSDLIYEFMKKGDERLLE
EAERLLEEVER,
SDKEWILQKIYEIMRL-Cyclo[CDELGHAEAC]MRVSDLIYEFMKKG,
DKEWILQKIYEIMRL-Cyclo[CDELGHAEAC]-MRVSDLIYEFMKKG
ILQKIYEIMRL-Cyclo[CDELGHAEAC]-MRVSDLIYEFMKKG,
QKIYEIMRL-Cyclo[CDELGHAEAC]-MRVSDLIYEFMKKG,
DKEWILQKIYEIMRL-Cyclo[CDELGHAEAC]MRVSDLIYEFMKK,
DKEWILQKIYEI-Cyclo[CRLLDELGHAEASMRC]-SDLIYEFMKKG,
ILQKIYEI-Cyclo[CRLLDELGHAEASMRC]-SDLIYEFMKKG,
or
QKIYEI-Cyclo[CRLLDELGHAEASMRC]-SDLIYEFMKKG.

### Exemplary Linkers (L)

In any aspect or embodiment described herein, the L is an optionally substituted C₁-C₇₀ alkylene group, an optionally substituted C₁-C₆₀ alkylene group, or an optionally substituted C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, C₆₀, C₆₁, C₆₂, C₆₃, C₆₄, C₆₅, C₆₆, C₆₇, C₆₈, C₆₉, or C₇₀ alkylene), wherein:
the L optionally comprises one to fourteen -C₁₋₂₅ alkylene-ABM covalently linked to the C₁₋₇₀ alkynelene group; and
the L optionally comprises one to fourteen -C₁₋₂₅ alkylene-TBM covalently linked to the C₁₋₇₀ alkynelene group;
each CH₂ group is optionally replaced with a group independently selected from CR^{L1}R^{L2}, O, S, SO, SO₂, NH, CO, CH═CH, C≡C, 5-7 membered heterocycle, and 3-8 membered carbocycle;
each R^{L1} is independently selected from H, an amino acid side chain, or an optionally substituted linear or branched C₁₋₅ alkyl with one or two CH₂ groups are optionally replaced with a group independently selected from O, S, SO, SO₂, or NH, wherein the C₁₋₅ alkyl group is optionally substituted with a hydroxyl, —NH₂, ═NH₂, —NH₃, oxo (═O), an optionally substituted 5-7 membered carbocycle or aryl, or an optionally substituted 3-10 membered heterocycle or heteroaryl;
each R^{L2} is independently selected from H and optionally substituted linear or branched C₁₋₄ alkyl (e.g., methyl);
one or two CH₂ groups of the C₁₋₅₀ alkylene, the C₁₋₂₅ alkylene-ABM, the C₁₋₂₅ alkylene-TBM, or a combination thereof is optionally replaced with a [CON], wherein the [CON] is a bond or an optional chemical moiety that forms from a reaction production; and
the L is optionally substituted with one or more (e.g., 1, 2, 3, 4, 5, 6, 7, 8, 9, 10 or more) groups independently selected from H, halogen, hydroxyl, cyano, oxo (═O), amino, imino (═NH), —COOH, C₁₋₄ alkyl, C₁₋₄haloalkyl, and C₁₋₄alkoxy.

In any aspect or embodiment described herein, each amino acid side chain is independently H, —CH₂SH, —CH₂SeH, methyl, isopropyl,

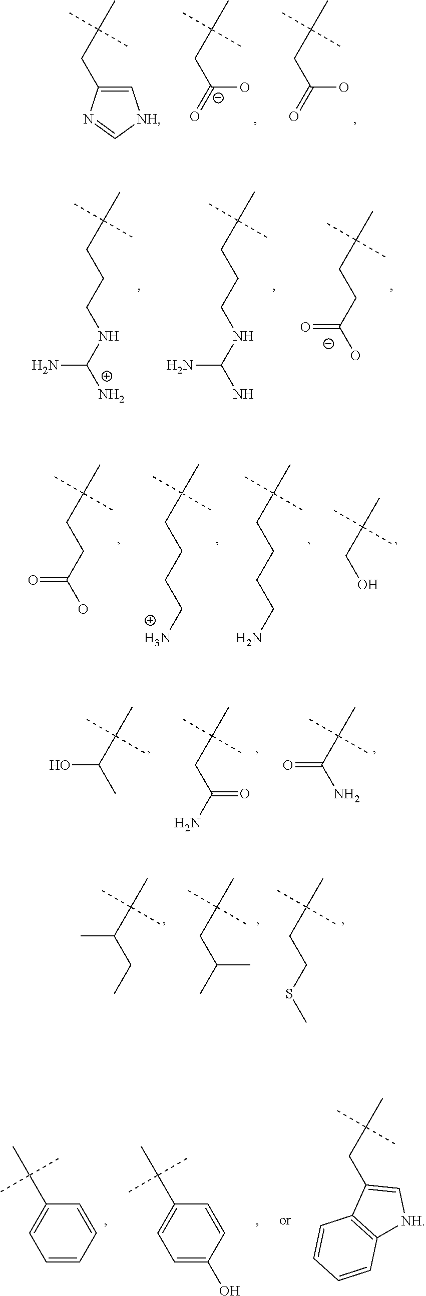

The term “connector”, symbolized in the generic formulas by [CON], is used to describe a bond or an optional chemical moiety included in bifunctional compounds of this disclosure which forms from the reaction product to covalently link two groups selected from ABM, L, and TBM. The connector group is often the resulting moiety which forms from the facile condensation of two or more separate chemical fragments which contain reactive groups which can provide connector groups as otherwise described to produce bifunctional or multifunctional compounds of this disclosure. It is noted that a connector may be distinguishable from a L in that the [CON] is the result of a specific chemistry which is used to provide bifunctional compounds of this disclosure wherein the reaction product of these groups results in an identifiable connector group or part of a connector group which is distinguishable from the L group, although in certain instances, the connector group is incorporated into and integral with the L group as otherwise described herein. It is noted also that a connector group may be linked to or part of a number of linkers to provide multifunctionality (i.e., more than one ABM and/or more than one TBM) within the same molecule. It is noted that there may be some overlap between the description of the connector group and the L group such that the connector group is actually incorporated or forms part of the L, especially with respect to more common connector groups such as amide groups, oxygen (ether), sulfur (thioether) or amine linkages, urea or carbonate -OC(O)O— groups or as otherwise described herein. It is further noted that a [CON](or L) may be connected to ABM, TBM, or L at positions which are represented as being linked to another group using the symbol or

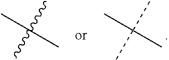

Where two or more such groups are present in a linker or connector, any of an ABM, a L, or a TBM group may be bonded to such a group. Where that symbol is not used, the L may be at one or more positions of a moiety.

In any aspect or embodiment described herein, one or more [CON] is a bond.

It is noted that each connector may be extended with one or more methylene groups to facilitate connection to a L group, another CON group, a TBM, or a ABM. It is noted that in certain instances, within context the diamide group may also function independently as a L group.

In any aspect or embodiment described herein, each [CON], independently, is or comprises:

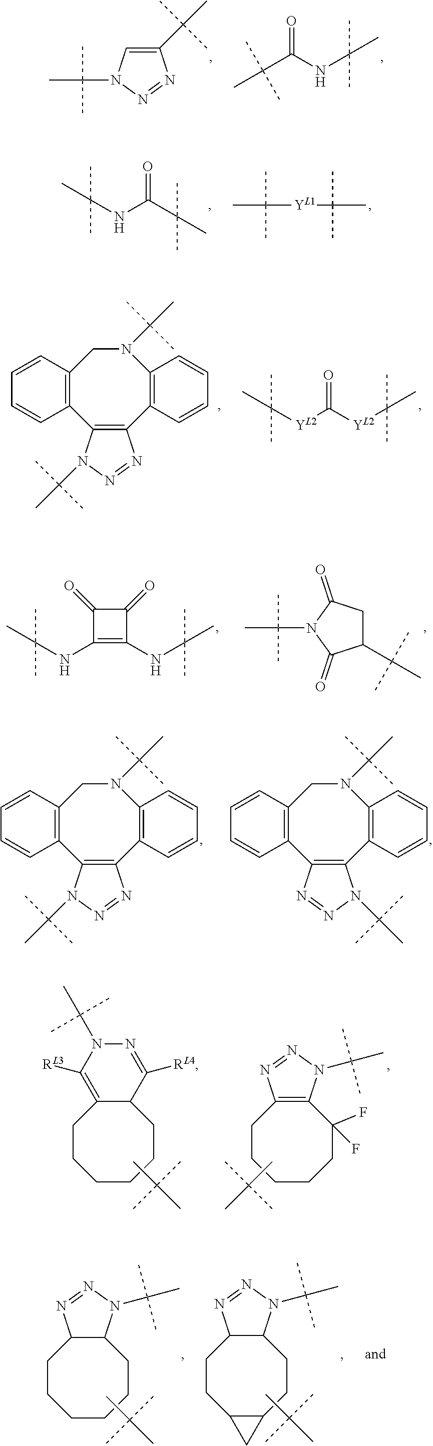

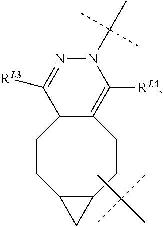

wherein:
each Y¹ is independently CH₂, O, S, NR^{L5} (e.g. NC₁-C₃alkyl)), C(O), S(O), S(O)₂, —S(O)₂°, —OS(O)₂, or OS(O)₂°;
each Y² is independently O, S, or NR^{L5};
each R^{L5} is independently H, C₁-C₃ alkyl, C₁-C₃ alkanoyl, or —C(O)(C₁-C₃alkyl) group;
each R^{L6} is independently H or C₁-C₃ alkyl group (preferably H); and
R^{L3} and R^{L4} are each independently H, methyl, a bond (e.g., for attachment to ABM, L, or TBM); or a dioxo or diamide group according to the structure:

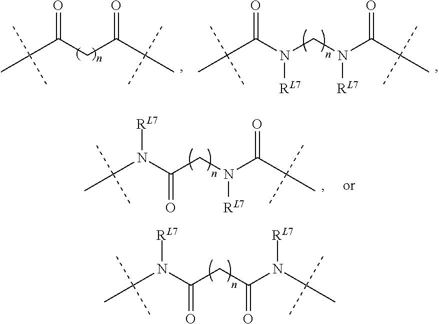

(e.g., one end is for attachment to ABM, L, or TBM), wherein each R^{L7} is independently H or a C₁₋₃ alkyl group (preferably H); and n of [CON] is an integer from 1 to 10 (n is preferably 1, 2, 3, 4, 5, or 6).

In any aspect or embodiment described herein, the L comprises amino acids (e.g., one or more amino acids including G, E, A, K, Q, A, P, or a combination thereof, such as -G_{Ln}-, -QAP_{Ln}-, -GQAP_{Ln}-, -PAS_{Ln}-, or a combination thereof, wherein Ln is an integer from 1 to 15 (such as, 1, 2, 3, 4, 5, 6, 7, 8, 9, 10, 11, 12, 13, 14, or 15).

In any aspect or embodiment described herein, one or more L, independently, is or comprises an optionally substituted L selected from: -GG-, -GGG-, -GGGG-, -GGGGGG-, -GGGGGGG-, -GGGGGGGG-, EAAAKEAAAK-, -QAP-, —[NH—(CH₂CH₂O)—CH₂CH₂CO]₂—, —CO—(CH₂CH₂O)₈—CH₂CH₂CONH—(CH₂CH₂O)₈—CH₂CH₂CO—, GQAPGQAP-, —NH—(CH₂CH₂O)₈—CH₂CH₂CO—, —[CO—(CH₂CH₂O)₈—CH₂CH₂CO]—, —NH-PEG₂-NH—, —NH—CH₂CH₂CH₂—CO—, —NH—(CH₂CH₂O)₄—CH₂CH₂—, —[NH—(CH₂CH₂O)₈—CH₂CH₂CO]₂O—, -PASPASPASPAS-, —[NH—(CH₂CH₂O)₈—CH₂CH₂C═O]₂—(CH₂CH₂O)₂—CH₂CH₂COO— or —[NH—(CH₂CH₂O)₈—CH₂CH₂C═O]₂—(CH₂CH₂O)₂—CH₂CH₂CO—,

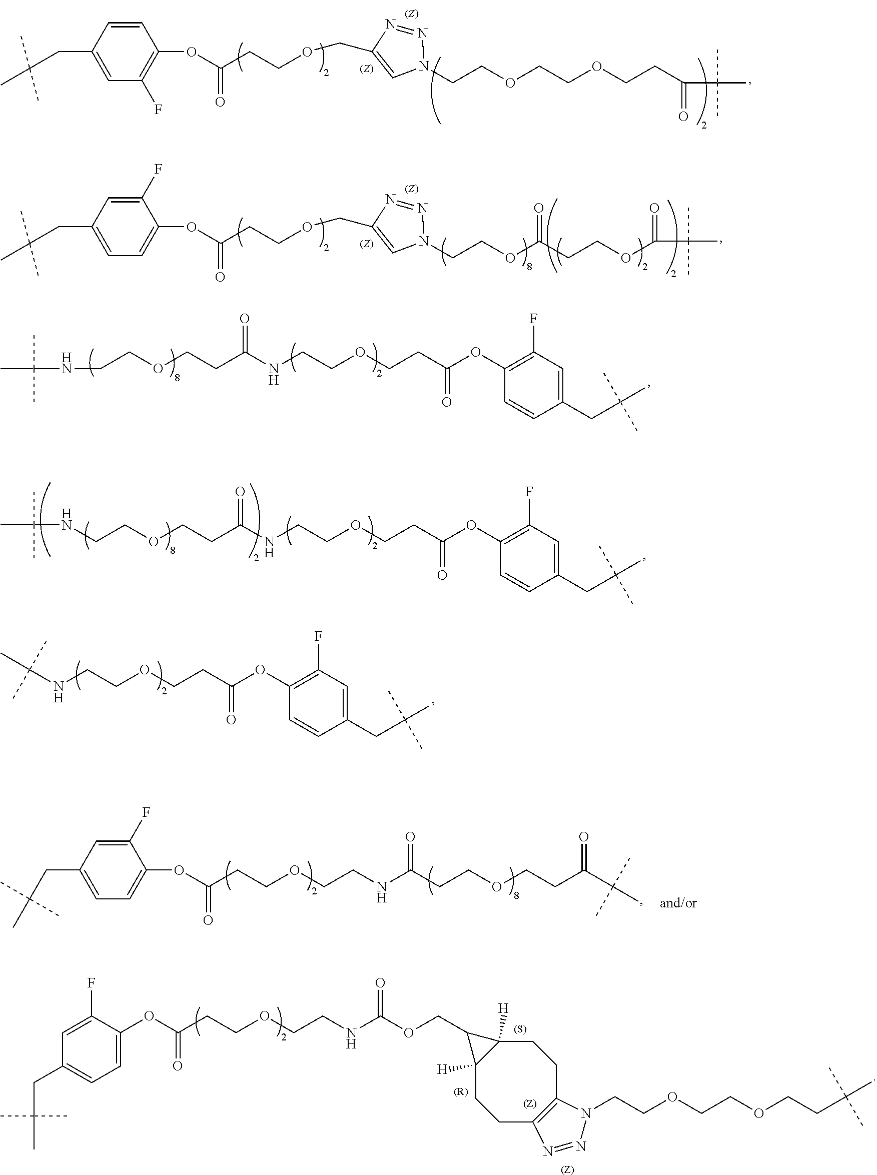

In any aspect or embodiment described herein, one or more L is a bond.

In any aspect or embodiment described herein, one or more L, independently, is or comprises a L from an exemplary compound of the present disclosure.

### Amino Acids

In any aspect or embodiment described herein, amino acid moiety or moieties are or comprise a proteinogenic (natural) amino acid. In any aspect or embodiment described herein, amino acid moiety or moieties are or comprise a non- proteinogenic (unnatural) amino acid.

In any aspect or embodiment described herein, an amino acid has the chemical structure:

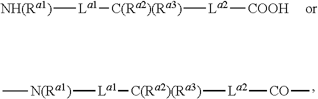

or a pharmaceutically acceptable salt thereof, wherein each variable is independent as described in the present disclosure. In any aspect or embodiment described herein, L^{a1}, L^{a2}, or both is a covalent bond.

In any aspect or embodiment described herein, an amino acid has the chemical structure:

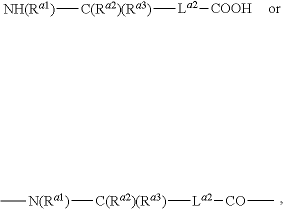

or a pharmaceutically acceptable salt thereof, wherein each variable is independent as described in the present disclosure.

In any aspect or embodiment described herein, an amino acid has the chemical structure:

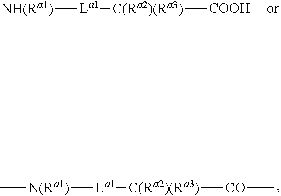

or a pharmaceutically acceptable salt thereof, wherein each variable is independent as described in the present disclosure.

In any aspect or embodiment described herein,
each L^{a1}and L^{a2} is independently L^{a};
each R^{a1}, R^{a2}, and R^{a3} is independently -L^{a}-R′;
each L^{a} is independently a covalent bond, or an optionally substituted bivalent group selected from C₁-C₂₀ aliphatic and C₁-C₂₀ heteroaliphatic having 1-5 heteroatoms, wherein one or more methylene units of the bivalent group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—; each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₁₋₃₀ aryl, C₁₋₃₀ arylaliphatic, C₁₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms,
wherein each heteroatom of the amino acid is independently selected from oxygen, nitrogen, sulfur, phosphorus, and silicon.

### Cellular Immunotherapy

Various immune cells, particularly NK cells, may be utilized together with compounds/agents described herein to treat various conditions, disorders, or diseases including coronavirus infections or coronavirus infected cells. Such cells may be administered prior to, concurrently with, and/or subsequent to compounds/agents described herein (e.g., ARMs). In any aspect or embodiment described herein, such cells (e.g., NK cells) are administered concurrently with a compound/agent (e.g., an ARM) in the same composition comprising both NK cells and a compound/ARM. In any aspect or embodiment described herein, such cells (e.g., NK cells) are administered concurrently with a compound/agent (e.g., an ARM) in separate compositions (e.g., one composition comprising NK cells but no compounds/ARMs), and one composition comprising a compound/ARM, but no NK cells.

As appreciated by those skilled in the art, useful immune cells (such as NK cells) may be from various sources and/or be engineered in a number of ways. For example, in any aspect or embodiment described herein, NK cells are derived from stem cells. In any aspect or embodiment described herein, NK cells are derived from iPSC lines. In any aspect or embodiment described herein, NK cells are derived from a clonal master iPSC line. In any aspect or embodiment described herein, NK cells are engineered to express certain receptors (e.g., a high-affinity), optionally non-cleavable CD16 receptor. In any aspect or embodiment described herein, NK cells are engineered to express chimeric antigen receptors (CARs) (e.g., in any aspect or embodiment described herein, NK cells may be engineered to express anti-CD19 CAR). In any aspect or embodiment described herein, NK cells are CAR-NK cells. In any aspect or embodiment described herein, NK cells are engineered to express cytokine receptor. In any aspect or embodiment described herein, NK cells comprise a IL-15 receptor fusion that enhances the persistence and expansion capabilities without requiring co-administration of cytokine support. In any aspect or embodiment described herein, NK cells are engineered to prevent expression of certain cell proteins (e.g., certain cell surface proteins). In any aspect or embodiment described herein, NK cells are engineered to prevent expression of CD38. In any aspect or embodiment described herein, NK cells are derived from placenta. In any aspect or embodiment described herein, NK cells are donor NK cells. In any aspect or embodiment described herein, NK cells are haploidentical donor NK cells. In any aspect or embodiment described herein, NK cells are mismatched donor NK cells. In any aspect or embodiment described herein, NK cells are related donor NK cells (e.g., mismatched related donor NK cells). In any aspect or embodiment described herein, NK cells are unrelated donor NK cells. In any aspect or embodiment described herein, NK cells are derived from a subject (e.g., a patient). In any aspect or embodiment described herein, provided technologies comprise an innate cell engager (e.g., an innate cell engager binding to innate cells (e.g., NK cells and macrophages) while binding simultaneously to specific coronavirus infected cells. In any aspect or embodiment described herein, NK cells are derived from cord blood stem and progenitor cells. In any aspect or embodiment described herein, NK cells are derived with modulation of a signaling pathway (e.g., the Notch signaling pathway). In any aspect or embodiment described herein, nanoparticles are utilized to improve and/or sustain growth of NK cells. In any aspect or embodiment described herein, NK cells are generated ex vivo. In any aspect or embodiment described herein, NK cells may be cryopreserved and stored in multiple doses as off-the-shelf cell therapy. Examples of certain such technologies include those utilized by Fate Therapeutics, NantKwest Inc., Celularity, Inc., GC Pharma, Sorrento Therapeutics, Inc., Affimed GmbH/MD Anderson Cancer Center, Gamida Cell Ltd., Nohla Therapeutics, Kiadis Pharma N.V., etc. Those skilled in the art will appreciate that, which they can be optionally utilized, antibodies and/or CARs toward specific antigens utilized in certain such technologies may not be required in provided technologies comprising compounds/ARMs as described herein.

### Cell Populations/Preparations

In any aspect or embodiment described herein, useful cells (e.g., NK cells) are processed before administration to a subject. In any aspect or embodiment described herein, NK cells are enriched, pre-activated and/or expanded in vitro before administration to a subject. In some embodiments, a composition comprising NK cells for administration is enriched for NK cells compare to a reference composition (e.g., blood from a subject to whom such composition is to be administered). In any aspect or embodiment described herein, an enrichment is or comprises an increased number of cells in a unit volume, e.g., per mL. In any aspect or embodiment described herein, an enrichment is or comprises an increased percentage of NK cells in such composition. In any aspect or embodiment described herein, an enrichment is or comprises an increased number of cells in a unit volume and an increased percentage of NK cells is a composition. In any aspect or embodiment described herein, a composition is a substantially pure composition of NK cells in that the non-NK cells in the composition are “impurities” from the production process (e.g., due to incomplete isolation, purification, etc.). As appreciated by those skilled in the art, chemical and biological processes rarely, if ever, go to completion and/or proceed to completeness or achieve or avoid an absolute result. The term “substantially” is therefore used in the present disclosure to capture the potential lack of completeness inherent in many biological and/or chemical phenomena.

Various technologies for enriching NK cells are available in the art and can be utilized in accordance with the present disclosure. For example, in any aspect or embodiment described herein, NK cells are isolated using leukapheresis optionally with purification steps such as CD3 depletion and/or CD56 positive selection. In any aspect or embodiment described herein, compared to a reference population of NK cells (e.g., NK cells in the blood of a donor or a subject), NK cells (e.g., pre-activated, memory-like NK cells) are enriched for CD56 and depleted from CD3 expressing cells.

Before administration to a subject, NK cells are typically pre-activated—e.g., preactivation with cytokines, such as IL-12, IL-15, and IL-18 for a suitable period of time (e.g., 1, 2, 3, 4, 5, 6, 7, 8, 9, 10, 11, 12, 13, 14, 15, 16, 17, 18, 19, 20, 21, 22, 23, 24, or 25, or more hours, e.g., about 16 hours). In any aspect or embodiment described herein, prior to administration, NK cells are treated with cytokines. In any aspect or embodiment described herein, NK cells are treated with a suitable amount of IL-12 (e.g., about 10 ng/mL), IL-15 (e.g., 50 ng/mL), and IL-18 (e.g., 50 ng/mL) for a suitable period of time (e.g., 1, 2, 3, 4, 5, 6, 7, 8, 9, 10, 11, 12, 13, 14, 15, 16, 17, 18, 19, 20, 21, 22, 23, 24, or 25, or more hours, e.g., about 16 hours) so that they are pre-activated. Conditions for preactivation of NK cells, and pre-activated NK cells may be assessed through assessment of one or more makers (e.g., CD94, NKG2A, NKp46, CD25, NKp30, NKp44, CD62L, CD27, TRAIL, cytotoxic molecules proforin and granzyme B, etc). Certain useful markers are described in Romee et al., Blood 120, 4751-4760, (2012); Leong et al., Biol. Blood Marrow Transplant. 20, 463-473 (2014); Romee et al., Sci Transl Med. 2016 Sep. 21; 8(357):357ra123. Doi: 10.1126/scitranslmed.aaf2341; etc. In any aspect or embodiment described herein, NK cells are primary human NK cells differentiated in vitro into memory-like NK cells via pre-activation with IL-12, IL-15, and IL-18.

Among other things, such cytokine-induced memory like NK cells exhibit enhanced responses to cytokine or activating receptor restimulation for weeks to months after preactivation, and can be particularly useful for treating various conditions, disorders or diseases including cancer. In any aspect or embodiment described herein, such pre-activated memory-like NK cells have enhanced interferon-y (IFN-γ) production and/or cytotoxicity against target cells (e.g., coronavirus infected cells). Exemplary procedures for preparing, assessing, and administering such NK cells can be found in Romee et al., Blood 120, 4751-4760, (2012); Leong et al., Biol. Blood Marrow Transplant. 20, 463-473 (2014); Romee et al., Sci Transl Med. 2016 Sep. 21; 8(357):357ra123. Doi: 10.1126/scitranslmed.aaf2341; etc. In any aspect or embodiment described herein, such NK cells have enhanced proliferation, express or express higher level of high-affinity IL-2 receptor aβγ, and/or provide increased IFN-γ production after re-stimulation (e.g., with cytokines or via activating receptors, etc.) compared to comparable reference NK cells, e.g., NK cells that are similarly prepared but are not pre-activated (e.g., not-treated with cytokines, treated with low-dose IL-15 only, etc).

Pre-activated memory-like NK cells may have significant differences in expression of a number of proteins compared to reference NK cells, e.g., CD94, NKG2A, NKp46, CD25, NKp30, NKp44, CD62L, CD27, TRAIL, cytotoxic molecules proforin and granzyme B, etc. In any aspect or embodiment described herein, such NK cells display a decrease in NKp80 expression. Such markers, among other things, may be utilized to enrich, purify and/or identify memory-like NK cells, either individually or in combination. Exemplary useful markers may be identified using various technologies, e.g., mass cytometry and multidimensional analyses. Exemplary procedures for identifying proteins that can distinguish pre-activated memory-like NK cells from reference (e.g., control) NK cells and exemplary such proteins are described in Romee et al., Sci Transl Med. 2016 Sep. 21; 8(357):357ra123. Doi: 10.1126/scitranslmed.aaf2341.

In any aspect or embodiment described herein, immune cells (e.g., NK cells) are contacted with a GSK3α/β inhibitor. In any aspect or embodiment described herein, such cells are pre-activated by a GSK3α/β inhibitor.

In any aspect or embodiment described herein, immune cells are cytokine-induced memory-like NK cells. In any aspect or embodiment described herein, immune cells are IL-12, IL-15, and IL-18-preactivated NK cells. In any aspect or embodiment described herein, immune cells are allogeneic, HLA-haploidentical, IL-12, IL-15, and IL-18-preactivated NK cells. Without the intention to be limited by any those, memory-like NK cells after IL-12, IL-15 and IL-18 preactivation may result from a process comprising differentiation that yields a long-term alteration of functional capacity. Memory-like NK cells with such functional capacity may be prepared by treatment with other agents (e.g., other cytokines, or other small molecules, peptides, proteins, etc.). Technologies (e.g., protein markers, assays, etc.) for assessing treatment with such as agents and/or result NK cells are available in the art and may be utilized in accordance with the present disclosure (e.g., those described in Romee et al., Sci Transl Med. 2016 Sep. 21; 8(357):357ra123. Doi: 10.1126/scitranslmed.aaf2341). In any aspect or embodiment described herein, memory-like NK cells have increased expression of inhibitory, activating, and cytokine receptors (e.g., CD94/NKG2A, NKp30, NKp44, NKp46, NKG2D, CD62L, and CD25, etc.), and/or decreased expression of certain proteins (e.g., NKp80). In any aspect or embodiment described herein, levels of certain receptors are substantially unchanged (e.g., KIR, CD57, NKG2C, DNAM-1, CD137, CD11b, etc.). In any aspect or embodiment described herein, changes in activating, inhibitory, cytokine and adhesion receptors are consistent with differentiation. In some embodiments, pre-activation by cytokines provide epigenetic modulation of certain genes, e.g., IFN-γ. For example, in any aspect or embodiment described herein, IL-12, IL-15, and IL-18 activation (e.g., for 5 days) result in reduced methylation of the IFN-γ conserved noncoding sequence 1 locus in human NK cells. In any aspect or embodiment described herein, certain preactivated NK cells (e.g., cytokine-induced) cells exist at an enhanced activation state without substantial differentiation.

In any aspect or embodiment described herein, NK cells are administered within 1, 2, 3, 4, 5, 6, 7, 8, 9, 10, 11, 12, 13, 14, 15, 16, 17, 18, 19, or 20 weeks, or 4, 5, 6, 7, 8, 9, 10, 11, or months, after pre-activation.

Among other things, the present disclosure encompasses the recognition that such NK cells, with their enhanced properties and/or activities which may provide enhanced inhibition or killing of cancer cells, may lead to more and/or more severe side effects and/or toxicities (e.g., off-target toxicities against normal cells). Thus, in any aspect or embodiment described herein, the present disclosure provides technologies for reducing such side effects and/or toxicities, comprising administering one or more agents, particularly ARMs, which, among other things, can promote, encourage, and/or enhance interactions of NK cells with their targets and reduce side effects and/or toxicities associated with NK cell administration.

In any aspect or embodiment described herein, NK cells are expanded (e.g., expanded prior to, during, and/or after preactivation). In any aspect or embodiment described herein, NK cells are purified before administration to remove, e.g., certain medium (or components thereof), activating cytokines, etc.

In any aspect or embodiment described herein, NK cells to be administered to a subject are isolated and/or expanded from the subject (autologous). In any aspect or embodiment described herein, NK cells to be administered to a subject are isolated and/or expanded from another subject (allogeneic; e.g., from a HLA-haploidentical donor).

In any aspect or embodiment described herein, NK cells, as described above, are treated (e.g., isolation, purification, pre-activation, etc.) before administration. In any aspect or embodiment described herein, a NK cell composition for administration comprise certain artificial medium or components thereof, e.g., complete RPMI-1640 medium containing L-glutamine, HEPES, NEAA, penicillin/streptomycin, and 10% human AB serum, optionally supplemented with rhIL-15 (1 ng/mL) to support survival.

### Administration

Immune cells (e.g., pre-activated memory-like NK cells) may be formulated in various formats as known in the art for administration to a subject. Depending on subjects and/or conditions, coronavirus infection to be treated, suitable amounts of cells are administered to provide clinical benefits. In any aspect or embodiment described herein, a cell dose level is 0.01-100, 0.1-50, 0.1-20, 0.1-10, 0.05, 0.1, 0.2, 0.3, 0.3, 0.5, 1, 2, 3, 4, 5, 6, 7, 8, 9, 10, 15, 20, 25, 30, 35, 40, 45, 50, 60, 70, 80 or 90 million per kilogram. In any aspect or embodiment described herein, a dose level is 0.5×10⁶ per kg. In any aspect or embodiment described herein, a dose level is 1×10⁶ per kg. In any aspect or embodiment described herein, a dose level is no more than 10×10⁶ per kg. As appreciated by those skilled in the art, pre-activated memory-like NK cells may be administered at a lower level to achieve the same or more clinical benefits, or provide more clinical benefits when administered at the same level when compared to un-activated NK cells, or pre-activated NK cells that are not memory-like (e.g., treated by only IL-2 or IL-15 for, e.g., overnight).

In any aspect or embodiment described herein, cells are administered via intravenous infusion. In any aspect or embodiment described herein, compositions comprising cells, e.g., pre-activated NK cells, are administered via infusion. In any aspect or embodiment described herein, such compositions are administered by adoptive transfer.

In any aspect or embodiment described herein, immune cells (e.g., pre-activated NK cells) are administered prior to administration of compounds/agents (such as ARMs). In any aspect or embodiment described herein, they are administered concurrently with compounds/agents (such as ARMs). In any aspect or embodiment described herein, they are administered subsequent to compounds/agents (such as ARMs). In any aspect or embodiment described herein, when not administered concurrently, immune cells (e.g., pre-activated NK cells) and compound/agents (such as ARMs) are administered in such manners that a subject is exposed to both. Certain technologies for administration of NK cells are described in Miller et al., Blood 105, 3051-3057 (2005) and may be utilized in accordance with the present disclosure.

In any aspect or embodiment described herein, after or concurrently with administration of immune cells, certain cytokines are administered to support administered immune cells. In any aspect or embodiment described herein, rhIL-2, typically a low dose, is administered to support memory-like NK cells through their induced high-affinity IL-2Rαβγ. As an example, see Leong, et al., Biol. Blood Marrow Transplant, 20, 463-473 (2014).

In any aspect or embodiment described herein, donor memory-like NK cells are tracked in the blood of a subject with informative HLA, e.g., using donor- or patient-specific anti-HLA monoclonal antibodies. In any aspect or embodiment described herein, such cells peak in frequency at, e.g., 7 to 14 days after infusion, and may decrease in number after recipient T cell recovery. If desired, additional dose of such NK cells may be administered (in some embodiments, 1, 2, 3, 4, 5, 6, 7, 8, 9, 10 or more doses can be administered). In any aspect or embodiment described herein, memory-like NK cells comprise no less than about 10%, 15%, 20%, 25%, 30%, 35%, 40%, 45%, 50%, 60%, 70%, 80%, or 90% of blood NK cells at a time after administration (e.g., at day 7). In any aspect or embodiment described herein, the level is more than about 10%. In any aspect or embodiment described herein, the level is more than about 20%. In any aspect or embodiment described herein, the level is more than about 30%. In any aspect or embodiment described herein, the level is more than about 40%. In any aspect or embodiment described herein, the level is more than about 50%. In any aspect or embodiment described herein, the level is more than about 60%. In any aspect or embodiment described herein, the level is more than about 70%. In any aspect or embodiment described herein, the level is more than about 80%. In any aspect or embodiment described herein, the level is more than about 90%. In any aspect or embodiment described herein, counts of memory-like NK cells, e.g., in blood, increase at least 50, 100, 150, 200, 250, 300, 350, 400, 450, 500, 550, 600, 650, 700, 750, 800, 850, 900, 950, or 1000 fold comparing day 1 and a later time point (e.g., day 7). In any aspect or embodiment described herein, the increase is at least 100 fold. In any aspect or embodiment described herein, the increase is at least 200 fold. In any aspect or embodiment described herein, the increase is at least 300 fold. In any aspect or embodiment described herein, the increase is at least 400 fold. In any aspect or embodiment described herein, the increase is at least 500 fold. In any aspect or embodiment described herein, the increase is at least 600 fold. In any aspect or embodiment described herein, the increase is at least 700 fold. In any aspect or embodiment described herein, the increase is at least 800 fold. In any aspect or embodiment described herein, the increase is at least 900 fold. In any aspect or embodiment described herein, the increase is at least 1000 fold. Typically, percentages and absolute numbers (counts) of memory-like NK cells are also increased, e.g., to percentages (e.g., no less than about 10%, 15%, 20%, 25%, 30%, 35%, 40%, 45%, 50%, 60%, 70%, 80%, or 90%; in any aspect or embodiments described herein, at least 90%) and counts (e.g., 50, 100, 150, 200, 250, 300, 350, 400, 450, 500, 550, 600, 650, 700, 750, 800, 850, 900, 950, or 1000 fold or more; in any aspect or embodiment described herein, 100, 200, 300, 400, 500, 600 fold or more) described above after administration of memory-like NK cells (e.g., at day 5, 6, 7, 8, 9, 10, 11, 12, 13, and/or 14).

In any aspect or embodiment described herein, after administration memory-like NK cells have enhanced functionality, e.g., as demonstrated by increased IFN-γ production upon stimulation.

In any aspect or embodiment described herein, memory-like NK cells have increased proliferation (Ki-67+) after administration (e.g., at days 3 and/or 7).

In any aspect or embodiment described herein, pre-activated memory-like NK cells are cytokine-induced memory-like NK cells. In any aspect or embodiment described herein, memory-like NK cells express inhibitory KIR receptor. In any aspect or embodiment described herein, such cells can perform one or more functions (e.g., inhibition and/or killing of target cells such as cancer cells) regardless of KIR-ligand interactions.

In any aspect or embodiment described herein, administered immune cells (e.g., memory-like NK cells) are detectable in the blood and bone marrow of a subject for 1, 2, 3, 4, 5, 6, 7, 8, 9, 10 days, or 1, 2, 3, 4, 5, 6, 7, 8, 9, 10 weeks, or more. In any aspect or embodiment described herein, such cells proliferate and/or expand in vivo after administration to a subject. In any aspect or embodiment described herein, such cells differentiate in a subject in vivo and exhibit enhanced functionality against targets such as cancer cells (e.g., leukemia cells).

In any aspect or embodiment described herein, immune cells (e.g., pre-activated NK cells) are administered concurrently with a compound/agent (such as an ARM). In any aspect or embodiment described herein, they are administered in a single composition. In any aspect or embodiment described herein, they are administered as separate compositions.

In any aspect or embodiment described herein, together with ARMs, a suitable amount of antibodies may also be administered, either in the same composition or in separate compositions (and optionally may be administered prior to, concurrently with or subsequent to ARMs). In any aspect or embodiment described herein, a composition comprising an ARM, pre-activated NK cells, and optionally an antibody or a fragment thereof is administered. In any aspect or embodiment described herein, a composition comprising an ARM comprising a uABT, pre-activated NK cells, and optionally an IgM is administered. In any aspect or embodiment described herein, a provided composition comprising a complex comprising a NK cell, an antibody or a fragment thereof, and a compound/agent (such as an ARM). In any aspect or embodiment described herein, in such a complex A CD16a receptor of an NK cells interacts with an antibody or a fragment thereof, and an antibody binding moiety of a compound/agent (such as an ARM) interacts with the same antibody or a fragment thereof.

### Pharmaceutically Acceptable Compositions

In any aspect or embodiment described herein, the present disclosure provides a composition comprising immune cells (e.g., pre-activated memory-like NK cells) and a pharmaceutically acceptable carrier, excipient, adjuvant, or vehicle. In any aspect or embodiment described herein, the present disclosure provides a composition comprising one or more (e.g., 1, 2, 3, 4, 5, 6, or more) compound/agent or a pharmaceutically acceptable derivative thereof of the present disclosure and a pharmaceutically acceptable carrier, excipient, adjuvant, or vehicle. In any aspect or embodiment described herein, the present disclosure provides a composition comprising immune cells, one or more (e.g., 1, 2, 3, 4, 5, 6, or more) compounds/agent and a pharmaceutically acceptable derivative thereof and a pharmaceutically acceptable carrier, adjuvant, or vehicle. In any aspect or embodiment described herein, immune cells and useful compounds/agents (e.g., ARMs) are provided in separate compositions. In any aspect or embodiment described herein, immune cells and useful compounds/agents (e.g., ARMs) are provided in the same compositions.

In any aspect or embodiment described herein, immune cells are typically administered at amounts sufficient to provide therapeutic benefits without severe side effects. For example, in any aspect or embodiment described herein, immune cells are administered at 0.01-100, 0.1-50, 0.1-20, 0.1-10, 0.05, 0.1, 0.2, 0.3, 0.3, 0.5, 1, 2, 3, 4, 5, 6, 7, 8, 9, 10, 15, 20, 25, 30, 35, 40, 45, 50, 60, 70, 80 or 90 million per kilogram of body weight of a subject.

Compounds/agents (e.g., ARMs) are typically administered at amounts that are effective to redirect endogenous antibodies (e.g., endogenous antibodies) and/or fragments thereof selectively to target cells (e.g., coronavirus infected cells), thereby inducing antibody-directed, cell-mediated immunity (e.g., cytotoxicity). In any aspect or embodiment described herein, amounts of compounds in compositions of the present disclosure are such that they are effective to redirect endogenous antibodies selectively to coronavirus (e.g., SARS-CoV-2) infected cells, thereby inducing antibody-directed, cell-mediated cytotoxicity, in a biological sample or in a subject. In any aspect or embodiment described herein, an amount is about 0.01 to about 100 mg/kg body weight. In any aspect or embodiment described herein, an amount is about 0.01 to about 50 mg/kg body weight. In any aspect or embodiment described herein, an amount is from about 1 mg/kg to about 25 mg/kg.

In any aspect or embodiment described herein, a pharmaceutically acceptable carrier, excipient, adjuvant, or vehicle is a non-toxic carrier, excipient, adjuvant, or vehicle that does not destroy the pharmacological activity of the compound with which it is formulated. Pharmaceutically acceptable carriers, excipients, adjuvants, or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In any aspect or embodiment described herein, immune cells may be provided in a suitable medium.

In any aspect or embodiment described herein, a compound/agent may be provided as a pharmaceutically acceptable derivative, which can be a non-toxic salt, ester, salt of an ester, or other derivative of a compound of the present disclosure that, upon administration to a recipient, is capable of providing, either directly or indirectly, the compound/agent.

Compositions of the present disclosure may be administered orally, parenterally, by inhalation spray, topically, rectally, nasally, buccally, vaginally or via an implanted reservoir. In any aspect or embodiment described herein, parenteral administration includes subcutaneous, intravenous, intramuscular, intra-articular, intra-synovial, intrasternal, intrathecal, intrahepatic, intralesional and intracranial injection or infusion techniques. Preferably, compositions are administered orally, intraperitoneally or intravenously. Preferably, compositions comprising immune cells are administered intravenously. Sterile injectable forms of compositions of this present disclosure may be aqueous or oleaginous suspension. Suspensions may be formulated according to techniques known in the art using suitable dispersing or wetting agents and suspending agents. A sterile injectable preparation may also be a sterile injectable solution or suspension in a non-toxic parenterally acceptable diluent or solvent, for example as a solution in 1,3-butanediol. Among acceptable vehicles and solvents that may be employed are water, Ringer's solution and isotonic sodium chloride solution. In addition, sterile, fixed oils are conventionally employed as a solvent or suspending medium.

In any aspect or embodiment described herein, compounds/agents (e.g., ARMs) are formulated as compositions for delayed and/or sustained releases. Technologies for sustained release and/or slowed absorption are widely available in the art and can be utilized in accordance with the present disclosure.

In any aspect or embodiment described herein, bland fixed oil may be employed including synthetic mono- or di-glycerides. Fatty acids, such as oleic acid and its glyceride derivatives may be useful in the preparation of injectables, as may be natural pharmaceutically-acceptable oils, such as olive oil or castor oil, especially in their polyoxyethylated versions. Such oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of compounds/agents of the present disclosure (e.g., ARMs) may be orally administered in any orally acceptable dosage form (e.g., capsules, tablets, aqueous suspensions, or solutions). In the case of tablets for oral use, carriers commonly used include lactose and corn starch. Lubricating agents, such as magnesium stearate, may also be added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms, e.g., for oral administration include capsules, tablets, pills, powders, and granules. In any aspect or embodiment described herein, a compound/agent to be delivered (e.g., an ARM) is mixed with at least one inert, pharmaceutically acceptable excipient or carrier (such as sodium citrate or dicalcium phosphate), and/or (a) fillers or extenders (such as, starches, lactose, sucrose, glucose, mannitol, and/or silicic acid), (b) binders (such as, carboxymethylcellulose, alginates, gelatin, polyvinylpyrrolidinone, sucrose, and/or acacia), (c) humectants (such as, glycerol), (d) disintegrating agents (such as, agar-agar, calcium carbonate, potato or tapioca starch, alginic acid, certain silicates, and/or sodium carbonate), I solution retarding agents (such as, paraffin), (f) absorption accelerators (such as, quaternary ammonium compounds), (g) wetting agents (such as, cetyl alcohol and/or glycerol monostearate), (h) absorbents (such as, kaolin and/or bentonite clay), and (i) lubricants (such as, talc, calcium stearate, magnesium stearate, solid polyethylene glycols, sodium lauryl sulfate, and mixtures thereof). In the case of capsules, tablets and pills, the dosage form may also comprise buffering agents.

Solid compositions may also be employed as fillers in soft and hard-filled gelatin capsules using such excipients as lactose or milk sugar, as well as high molecular weight polyethylene glycols and the like. In any aspect or embodiment described herein, solid dosage forms of tablets, dragees, capsules, pills, and granules can be prepared with coatings and shells such as enteric coatings and other coatings well known in the pharmaceutical formulating art. In any aspect or embodiment described herein, solid compositions may optionally contain opacifying agents and can release the agents to be delivered only, or preferentially, in a certain part of a subject (e.g., a certain part of the intestinal tract), optionally, in a delayed manner. Examples of embedding compositions that can be used include polymeric substances and waxes.

In any aspect or embodiment described herein, compounds/agents (e.g., ARMs) can be in micro-encapsulated form with one or more excipients as noted above. In any aspect or embodiment described herein, solid dosage forms of tablets, dragees, capsules, pills, and granules can be prepared with coatings and shells such as enteric coatings, release controlling coatings and other coatings well known in the pharmaceutical formulating art. In such solid dosage forms the agents to be delivered may be admixed with at least one inert diluent such as sucrose, lactose or starch. Such dosage forms may also comprise, as in normal practice, additional substances, e.g., tableting lubricants and other tableting aids such as magnesium stearate and microcrystalline cellulose. In any aspect or embodiment described herein, in the case of capsules, tablets and pills, dosage forms may comprise buffering agents.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of the present disclosure may also be administered topically, especially when the target of treatment includes areas or organs readily accessible by topical application, including diseases of the eye, the skin, or the lower intestinal tract. Suitable topical formulations can be readily prepared for each of these areas or organs.

For topical applications, pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Pharmaceutically acceptable compositions of agents, e.g., ARMs, may be formulated for oral administration. Such formulations may be administered with or without food. In some embodiments, pharmaceutically acceptable compositions are administered without food. In other embodiments, pharmaceutically acceptable compositions are administered with food.

It should also be understood that a specific dosage and treatment regimen for a particular subject will depend upon a variety of factors, e.g., the activity of the specific immune cells and/or agents employed, the age, body weight, general health, sex, diet, time of administration, rate of excretion, drug combination, and the judgment of the treating physician and the severity of the particular disease being treated, etc.

In any aspect or embodiment described herein, the present disclosure provides a pharmaceutical composition comprising: one or more (e.g., 1, 2, 3, 4, 5, or more) compounds of the present disclosure, or a pharmaceutically acceptable salt thereof, and a pharmaceutically acceptable carrier, adjuvant, additive, excipient, vehicle, or a combination thereof.

In any aspect or embodiment described herein, the present disclosure provides a combination comprising: (1) one or more (e.g., 1, 2, 3, 4, 5, or more) compound of the present disclosure, or a pharmaceutically acceptable salt thereof, and, and (2) a plurality of natural killer cells.

In any aspect or embodiment described herein, the present disclosure provides a combination comprising: (1) one or more (e.g., 1, 2, 3, 4, 5, or more) compound of the present disclosure, or a pharmaceutically acceptable salt thereof, (2) a plurality of natural killer cells; and (3) a pharmaceutically acceptable carrier, adjuvant, additive, excipient, vehicle, or a combination thereof.

### Uses and Administration

Among other things, the present disclosure encompasses the recognition that certain immune cells (such as pre-activated memory-like NK cells) have improved properties and/or activities toward their targets (e.g., coronavirus infected cells), and can be particularly effective for treating coronavirus-related diseases and/or conditions due to their increased cytotoxicity, as well as a decrease side-effects associated with off-targeting. Thus, in any aspect or embodiment described herein, the present disclosure provides technologies which expose a subject to both such immune cells and compounds/agents (e.g., ARMs) that can facilitate targeting of immune cells to desired targets (e.g., coronavirus infected cells) and reduce side effects of such immune cell therapy.

In any aspect or embodiment described herein, useful compounds/agents direct antibodies (e.g., endogenous antibodies) selectively to diseased cells (e.g., coronavirus infected cells), thereby inducing and/or promoting an antibody-directed, cell-mediated immune response (e.g., cytotoxicity by immune cells, such as pre-activated memory-like NK cells or cytokine-induced memory-like NK cells). In any aspect or embodiment described herein, recruited antibodies comprise one or more endogenous antibodies. In any aspect or embodiment described herein, recruited antibodies have specificity toward one or more antigens (e.g., one or more antigens of a coronavirus, such as SARS-CoV-2). In any aspect or embodiment described herein, recruited antibodies have specificity toward one or more peptide antigens or proteins (e.g., one or more peptide antigens or proteins of a coronavirus, such as SARS-CoV-2). In any aspect or embodiment described herein, recruited antibodies are heterogeneous in that they are not antibodies toward the same antigen or protein.

In any aspect or embodiment described herein, useful compounds/agents (e.g., ARMs) trigger, generate encourage, and/or enhance one or more immune system activities toward a target. In any aspect or embodiment described herein, an immune system activity is or comprises ADCC by NK cells. In any aspect or embodiment described herein, an immune system activity is or comprises ADCC by pre-activated memory-like NK cells (e.g., cytokine-induced memory-like NK cells). In any aspect or embodiment described herein, a target is a coronavirus infected cell (e.g., a SARS-CoV-2 infected cell). In any aspect or embodiment described herein, a target is a coronavirus infected cell in a subject.

As described herein, immune cells (such as pre-activated memory-like NK cells (e.g., cytokine-induced memory-like NK cells)) and compounds/agents (such as ARMs) are administered such that a subject can be exposed to both. In any aspect or embodiment described herein, immune cells (or a dose thereof) are administered prior to a compound/ARM agent (or a dose thereof). In any aspect or embodiment described herein, immune cells (or a dose thereof) are administered subsequent to a compound/ARM agent (or a dose thereof). In any aspect or embodiment described herein, immune cells (or a dose thereof) are administered concurrently with a compound/ARM agent (or a dose thereof). When administered concurrently, immune cells and compounds/ARMs may be administered in one composition or as separate compositions. In any aspect or embodiment described herein, immune cells and compounds/ARMs are administered within 0, 1, 2, 3, 4, 5, 6, 7, 8, 9, 10, 11, 12, 13, 14, 15, 16, 17, 18, 19, 20, 21, 22, 23 or 24 hours, or 1, 2, 3, 4, 5, 6, 7, 8, 9, 10 days, or 1, 2, 3, 4, 5, 6, 7, 8, 9, 10, 11, 12, 13, 14, 15, 16, 17, 18, 19, 20 weeks, of each other. Immune cells and agents (e.g., compounds/ARMs of the present disclosure) may be independently administered as a single dose or multiple doses. In any aspect or embodiment described herein, administered immune cells (e.g., pre-activated memory-like NK cells) form complexes with administered compounds/ARMs together with targets (e.g., coronavirus infected cells) and antibodies (or fragments thereof) in a subject, wherein the antibodies (or fragments thereof) interact with the immune cells and compounds/ARMs, and the compounds/ARMs interact with the antibodies (or fragments thereof) and the targets (e.g., coronavirus infected cells).

In any aspect or embodiment described herein, “treating” or “treatment” as used herein includes reversing, alleviating, delaying the onset of, or inhibiting the progress of a condition, disorder or disease, or one or more symptoms thereof (such as, associated with or result from a coronavirus (e.g. SARS-CoV-2) infection). In any aspect or embodiment described herein, the condition, disorder, or disease is associated with or result from a coronavirus infection (e.g., a SARS-CoV-2 infection). In any aspect or embodiment described herein, treatment may be administered after one or more symptoms have developed. In any aspect or embodiment described herein, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In any aspect or embodiment described herein, the present invention provides a method for treating one or more disorders, diseases, and/or conditions wherein the disorder, disease, or condition is associated with or result from a coronavirus infection (e.g., a SARS-CoV-2 infection). Among other things, provided technologies (e.g., compounds, compositions, methods, etc.) are particularly useful for preventing and/or treating conditions, disorder, or diseases associated with or that result from a coronavirus infection (e.g., a SARS-CoV infection).

In any aspect or embodiment described herein, prior to administration of the present therapy (e.g., exposure to both administered pre-activated, memory-like NK cells and an ARM), a subject may have been treated with another therapy. In any aspect or embodiment described herein, a subject is susceptible to or suffering from relapsed or refractory coronavirus infection.

In any aspect or embodiment described herein, the present disclosure provides a method for inducing cell death, inhibiting cell growth, and/or reducing the number of cells in a system, the method comprising administering to the system one or more (e.g., 1, 2, 3, 4, 5, or more) compounds of the present disclosure, or a pharmaceutically acceptable salt thereof, wherein the number of cells in the system is reduced compared to the absence of the compound.

In any aspect or embodiment described herein, the method further comprises administering a plurality of natural killer cells, wherein the system is exposed to the plurality of natural killer cells and the compound, and the number of cells in the system is reduced compared to the absence of the natural killer cells and the compound.

In any aspect or embodiment described herein, the present disclosure provides a method of treating a coronavirus infection, the method comprising administering to a subject in need thereof one or more (e.g., 1, 2, 3, 4, 5, or more) compound of the present disclosure, or a pharmaceutically acceptable salt thereof, wherein the number of coronavirus infected cells is reduced as compared to the absence of the compound.

In any aspect or embodiment described herein, further comprises administering to the subject in need thereof a plurality of natural killer cells, wherein the subject is exposed to the plurality of natural killer cells and the compound, and the number of coronavirus infected cells in the subject is reduced as compared to the absence of the natural killer cells and the compound.

In any aspect or embodiment described herein, the system or the subject is a human, a non-human animal, or a non-human animal useful as a disease model.

In any aspect or embodiment described herein, the compound is administered prior to, concurrent with, and/or subsequent to the plurality of natural killer cells.

In any aspect or embodiment described herein, the plurality of natural killer cells are expanded natural killer cells, enriched natural killer cells, and/or pre-activated natural killer cells.

### Combination Therapies and Administration

In any aspect or embodiment described herein, provided technologies (e.g., those comprising immune cells (such as, memory-like NK cells) and compounds/ARMs) are administered in combination with one or more other therapies. In any aspect or embodiment described herein, in addition to immune cells (e.g., memory-like NK cells) and a compound/ARM agent, a subject is administered another therapy. In any aspect or embodiment described herein, a subject is suffering from a coronavirus infection, and such another therapy is anti-viral therapy (e.g., a therapy for a coronavirus infections, such as SARS-CoV-2).

Among other things, technologies of the present disclosure comprising immune cells (e.g., memory-like NK cells) and compounds/ARMs can be employed in combination with other therapies, that is, such technologies can be administered concurrently with, prior to, or subsequent to, one or more other therapeutic agents or medical procedures, particularly for treatment of various coronavirus infections.

When used in combination, compositions can be administered alone or in combination with one or more other therapeutic agents. Those additional agents may be administered separately, e.g., as part of a multiple dosage regimen. Alternatively, those agents may be part of a single dosage form, mixed together with a composition comprising immune cells and/or compounds/ARMs. Agents may be administered simultaneously, sequentially or within a period of time, e.g., 1, 2, 3, 4, 5, 6, 7, 8, 9, 10, 11, 12, 13, 14, 15, 16, 17, 18, 19, 20, 21, 22, 23, 24, or 25 hours, or 1, 2, 3, 4, 5, 6, 7, 8, 9, 10 days, or 1, 2, 3, 4, 5, 6, 7, 8, 9, 10 weeks, from one another. In any aspect or embodiment described herein, subjects are exposed to all agents (e.g., immune cells, compounds/ARMs of the present disclosure, and an additional agent) in a combination at the same time.

In any aspect or embodiment described herein, additional therapeutic agent and technologies comprising immune cells and compounds ARMs of the present disclosure act synergistically. Therefore, the amount of additional therapeutic agent in such combinations may be less than that required in a monotherapy utilizing only the therapeutic agent.

### EXEMPLIFICATION

Various technologies are available for preparing, characterizing, assessing and/or utilizing immune cells (e.g., memory-like NK cells) and ARMs for utilization in accordance with the present disclosure. Certain such technologies are described below. It will be appreciated that many other technologies are available and may be utilized.

### General Methods for Synthesis of Compounds of the Present Disclosure

As appreciated by those skilled in the art, many chemistry technologies are available in the art for preparing and characterizing compounds of the present disclosure. Certain compounds of the present disclosure are or comprise peptide entities. In any aspect or embodiment described herein, such peptides were prepared using standard, automated fluoroenylmethyloxycarbonyl (Fmoc) solid phase peptide synthesis procedures.

### Peptide Synthesis

In any aspect or embodiment described herein, peptides were synthesized using standard Fmoc chemistry. An exemplary procedure is described below.
1) Add DCM to a vessel containing CTC Resin (0.1 mmol) and Fmoc-Trp(Boc)-OH (42.5 mg, 0.08 mmol, 0.80 eq) with N₂ bubbling.
2) Add DIEA (4.0 eq) dropwise and mix for 2 hours.
3) Add MeOH (0.1 mL) and mix for 30 min.
4) Drain and wash with DMF for 5 times.
5) Add 20% piperidine/DMF and react on 30 min.
6) Drain and wash with DMF for 5 times.
7) Add Fmoc-amino acid solution and mix 30 seconds, then add activation buffer, N₂ bubbling for about 1 hour.
8) Repeat step 4 to 7 for next amino acid coupling.

In some embodiments, biotin-PEG8-CH₂CH₂COOH (1.5 eq) was coupled using HATU (1.5 eq) and DIEA (3.0 eq). In some embodiments, amino acids were coupled by 3 equivalent using HBTU (2.85 eq) and DIEA (6 eq). In some embodiments, 20% piperidine in DMF was used for Fmoc deprotection for a suitable period of time, e.g., 30 min. Coupling reactions were typically monitored by ninhydrin test. Resin was typically washed with DMF for 5 times.

### Peptide Cleavage and Purification

An exemplary procedure for peptide cleavage and purification is described below.
1) Add cleavage buffer (95% TFA/2.5% TIS/2.5% H₂O) to a flask containing the side chain protected peptide at room temperature and stir for 3 hours.
2) The peptide was precipitated with cold Isopropyl ether and centrifuged (3 min at 3000 rpm).
3) Isopropyl ether washes two additional times.
4) Dry the crude peptide under vacuum 2 hours.
5) Purify the crude peptide by Prep-HPLC (A: 0.1% TFA in H₂O, B: ACN) and lyophilized to give the final product (64.3 mg, 52.3% yield).

### Purification Conditions

In an exemplary procedure, reverse phase HPLC (Gilson 281) was carried out on Luna C18 (200×25 mm; 10 um) and Gemini C18 (150*30 mm; 5 um) in series. Solvent A: water with 0.075% trifluoroacetic acid; Solvent B: acetonitrile. Gradient: at room temperature, 15% of B to 45% of B within 60 min at 20 mL/min; then 90% B at 20 mL/min over 10 min, UV detection (wave length=215 nm). The peptide was lyophilized to give the desired product (64.3 mg, 52.3% yield) as a white solid.

All final compounds showed the correct mass for the desired compound.

### Abbreviations

The following abbreviations are used in the examples below.

| | | | |
|---|---|---|---|
| Alloc | Allyloxycarbonyl | HATU | Hexafluorophosphate |
| | | | Azabenzotriazole |
| | | | Tetramethyl |
| | | | Uronium |
| ACN | Acetonitrile | HBTU | Hexafluorophosphate |
| | | | Benzotriazole |
| | | | Tetramethyl |
| | | | Uronium |
| AcOH | Acetyl Alcohol | HFIP | Hexafluoro-2-propanol |
| CTC | 2-Chlorotrinitryl resin | HOBt | Hydroxybenzotriazole |
| Resin | | | |
| DCM | Dichloromethane | MeOH | Methanol |
| DIC | Diisopropylcarbodiimide | NMM | N-Methylmorpholine |
| DIEA | N,N- | Pbf | 2,2,4,6,7- |
| | Diisopropylethylamine | | Pentamethyl-2,3- |
| | | | dihydrobenzofuran- |
| | | | 5-sulfonyl |
| DMF | Dimethylformamide | TIS/TIPS | Triisoproyl Silane |
| EDCI | 1-Ethyl-3-(3'-dimehtyl | TFA | Trifluoroacetic acid |
| | aminopropyl)carbodiimide | | |
| Fmoc | Fluorenylmethyloxycarbonyl | | |

### Example 1. Procedure for Preparation of Compound 102 (Linear Peptide)

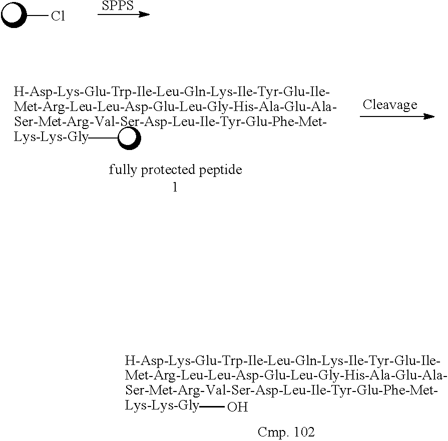

Compound 102 was synthesized using standard Fmoc chemistry (CTC resin).
1) Resin preparation: To the vessel containing CTC Resin (1.0 mmol, 1.0 g, 1.00 mmol/g) and Fmoc-Gly-OH (297.0 mg, 1.0 mmol, 1.00 eq) in DCM (10 mL) was added DIEA (4.00 eq) dropwise and mixed for 2 hrs with N₂ bubbling at 20° C. Then MeOH (1.0 mL) was added and bubbled with N₂ for another 30 mins. The resin was washed with DMF (20 mL)*5, followed by adding 20% piperidine in DMF (20 mL) and bubbled with N₂ for 30 mins at 20° C. for Fmoc deprotection. The mixture was filtered and the resin was washed with DMF (20 mL)*5 before proceeding to next step.
2) Coupling: A solution of Fmoc-Lys(Boc)-OH (1.41 g, 3.00 eq), HBTU (1.08 g, 2.85 eq) in DMF (10 mL) was added to the resin with N₂ bubbling. Then DIEA (6.00 eq) was added to the mixture dropwise and bubbled with N₂ for 30 mins at 20° C. The coupling reaction was monitored by ninhydrin test, if it showed colorless, the coupling was completed. The resin was then washed with DMF (20 mL) *5.
3) Deprotection: 20% piperidine in DMF (20 mL) was added to the resin and the mixture was bubbled with N₂ for 30 mins at 20° C. The deprotection reaction was monitored by ninhydrin test, if it showed blue or other brownish red, the reaction was completed. The resin was then washed with DMF (20 mL)*5.
4) Steps 2 and 3 were repeated for the following amino acids: number #3-12, in Table 1.
5) Coupling: A solution of Fmoc-Arg(Pbf)-OH (1.95 g, 3.00 eq), HATU (1.08 g, 2.85 eq) in DMF (10 mL) was added to the resin with N₂ bubbling for 30 mins at 20° C. Then DIEA (6.00 eq) was added to the mixture dropwise and bubbled with N₂ for 30 mins at 20° C. The coupling reaction was monitored by ninhydrin test, if it showed colorless, the coupling was completed. The resin was then washed with DMF (30 mL)*5.
6) Step 3 was repeated for Fmoc deprotection.
7) Steps 5 and 6 were repeated for amino acid numbers #14-39 in TABLE 1.
8) After the last position completed, the resin was then washed with DMF (20 mL)*5, MeOH (20 mL)*5, and then dried under vacuum to afford peptide resin intermediate 1 (SEQ ID NO:1) (CTC resin, 1 mmol, 9.5 g).

TABLE 1

| # | Materials | Coupling reagents |
|---|---|---|
| 1 | Fmoc-Gly-OH (1.00 eq) | DIEA (4.00 eq) |
| 2 | Fmoc-Lys(Boc)-OH (3.00 eq) | HBTU (2.85 eq) and DIEA (6.00 eq) |
| 3 | Fmoc-Lys(Boc)-OH (3.00 eq) | HBTU (2.85 eq) and DIEA (6.00 eq) |
| 4 | Fmoc-Met-OH (3.00 eq) | HBTU (2.85 eq) and DIEA (6.00 eq) |
| 5 | Fmoc-Phe-OH (3.00 eq) | HBTU (2.85 eq) and DIEA (6.00 eq) |
| 6 | Fmoc-Glu(OfBu)-OH (3.00 eq) | HBTU (2.85 eq) and DIEA (6.00 eq) |
| 7 | Fmoc-Tyr((Bu)-OH (3.00 eq) | HBTU (2.85 eq) and DIEA (6.00 eq) |
| 8 | Fmoc-Ile-OH (3.00 eq) | HBTU (2.85 eq) and DIEA (6.00 eq) |
| 9 | Fmoc-Leu-OH (3.00 eq) | HBTU (2.85 eq) and DIEA (6.00 eq) |
| 10 | Fmoc-Asp(OfBu)-OH (3.00 eq) | HBTU (2.85 eq) and DIEA (6.00 eq) |
| 11 | Fmoc-Ser(Bu)-OH (3.00 eq) | HBTU (2.85 eq) and DIEA (6.00 eq) |
| 12 | Fmoc-Val-OH (3.00 eq) | HBTU (2.85 eq) and DIEA (6.00 eq) |
| 13 | Fmoc-Arg(Pbf)-OH (3.00 eq) | HATU (2.85 eq) and DIEA (6.00 eq) |
| 14 | Fmoc-Met-OH (3.00 eq) | HATU (2.85 eq) and DIEA (6.00 eq) |
| 15 | Fmoc-Ser(Bu)-OH (3.00 eq) | HATU (2.85 eq) and DIEA (6.00 eq) |
| 16 | Fmoc-Ala-OH (3.00 eq) | HATU (2.85 eq) and DIEA (6.00 eq) |
| 17 | Fmoc-Glu(OtBu)-OH (3.00 eq) | HATU (2.85 eq) and DIEA (6.00 eq) |
| 18 | Fmoc-Ala-OH (3.00 eq) | HATU (2.85 eq) and DIEA (6.00 eq) |
| 19 | Fmoc-His(Trt)-OH (3.00 eq) | HATU (2.85 eq) and DIEA (6.00 eq) |
| 20 | Fmoc-Gly-OH (3.00 eq) | HATU (2.85 eq) and DIEA (6.00 eq) |
| 21 | Fmoc-Leu-OH (3.00 eq) | HATU (2.85 eq) and DIEA (6.00 eq) |
| 22 | Fmoc-Glu(OtBu)-OH (3.00 eq) | HATU (2.85 eq) and DIEA (6.00 eq) |
| 23 | Fmoc-Asp(OfBu)-OH (3.00 eq) | HATU (2.85 eq) and DIEA (6.00 eq) |
| 24 | Fmoc-Leu-OH (3.00 eq) | HATU (2.85 eq) and DIEA (6.00 eq) |
| 25 | Fmoc-Leu-OH (3.00 eq) | HATU (2.85 eq) and DIEA (6.00 eq) |
| 26 | Fmoc-Arg(Pbf)-OH (3.00 eq) | HATU (2.85 eq) and DIEA (6.00 eq) |
| 27 | Fmoc-Met-OH (3.00 eq) | HATU (2.85 eq) and DIEA (6.00 eq) |
| 28 | Fmoc-Ile-OH (3.00 eq) | HATU (2.85 eq) and DIEA (6.00 eq) |
| 29 | Fmoc-Glu(OtBu)-OH (3.00 eq) | HATU (2.85 eq) and DIEA (6.00 eq) |
| 30 | Fmoc-Tyr((Bu)-OH (3.00 eq) | HATU (2.85 eq) and DIEA (6.00 eq) |
| 31 | Fmoc-Ile-OH (3.00 eq) | HATU (2.85 eq) and DIEA (6.00 eq) |
| 32 | Fmoc-Lys(Boc)-OH (3.00 eq) | HATU (2.85 eq) and DIEA (6.00 eq) |
| 33 | Fmoc-Gln(Trt)-OH (3.00 eq) | HATU (2.85 eq) and DIEA (6.00 eq) |
| 34 | Fmoc-Leu-OH (3.00 eq) | HATU (2.85 eq) and DIEA (6.00 eq) |
| 35 | Fmoc-Ile-OH (3.00 eq) | HATU (2.85 eq) and DIEA (6.00 eq) |
| 36 | Fmoc-Trp-OH (3.00 eq) | HATU (2.85 eq) and DIEA (6.00 eq) |
| 37 | Fmoc-Glu(OfBu)-OH (3.00 eq) | HATU (2.85 eq) and DIEA (6.00 eq) |
| 38 | Fmoc-Lys(Boc)-OH (3.00 eq) | HATU (2.85 eq) and DIEA (6.00 eq) |
| 39 | Fmoc-Asp(OfBu)-OH (3.00 eq) | HATU (2.85 eq) and DIEA (6.00 eq) |

### Peptide Cleavage and Purification:

1) Cleavage buffer (95% TFA/2.5% TIS/2.5% H₂O, 20 mL) was added to the flask containing the side chain protected peptide resin (Intermediate 1 (SEQ ID NO:1), 0.2 mmol, 1.9 g) at room temperature and stirred for 2 hrs.
2) After filtration, the filtrate was collected.
3) The peptide was precipitated with cold isopropyl ether (100 mL) and centrifuged (3 mins at 3000 rpm).
4) Washed with isopropyl ether two additional times, and the crude peptide was dried under vacuum for 2 hrs to afford Compound 102 (1.2 g, crude, SEQ ID NO:41) as a white solid. 5) The crude was purified by prep-HPLC (acid condition, TFA), followed by lyophilization to afford Compound 102 (35.4 mg, 91.5% purity, 3.4500 yield) as a white solid. LCMS: RT=1.12 min, MS cal.: 4700.46, [M/3+H]⁺=1568.17, [M/4+H]⁺=1175.85, [M/5+H]⁺=941.32.

The dashed lines at the beginning and end of each line of peptide structure indicate a single covalent bond. E.g., in the structure below the methionine a-carbon is covalently bound to the carbonyl compound of the Arginine at the start of the next line of structure.

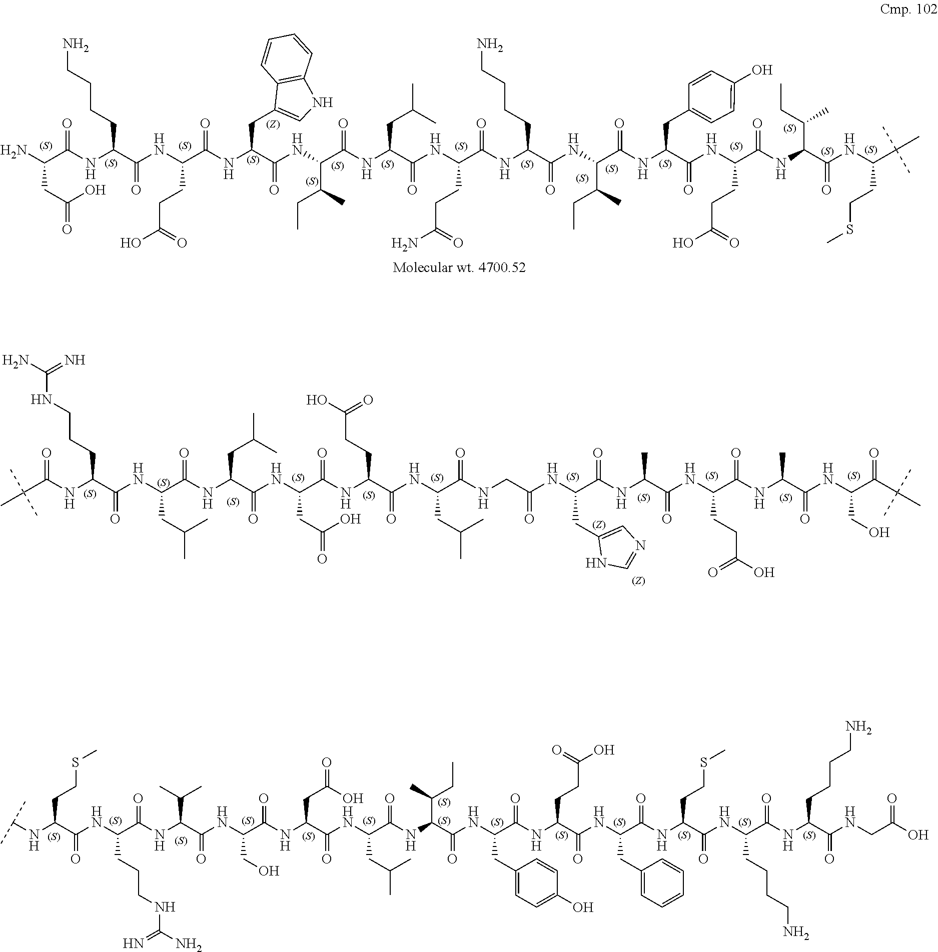

### Example 2. Procedure for Preparation of Compound 103 (Linear Peptide with Ac)

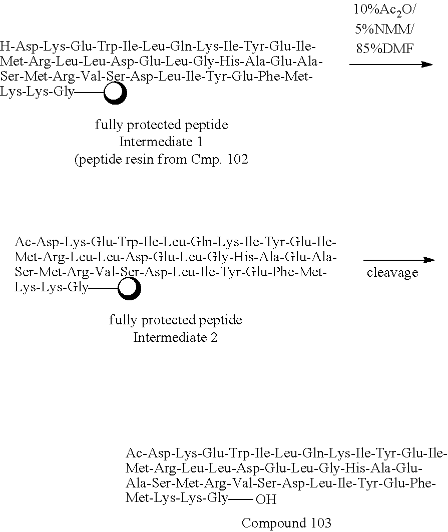

Peptide was synthesized using standard Fmoc chemistry, and SPPS was identical as in Cmp. 102.

Acetylation: A solution of 10% Ac₂O/5% NMM/85% DMF (30 mL) was added to resin (Intermediate 1, 0.2 mmol, 1.9 g, SEQ ID NO:1) and the mixture was bubbled with N₂ for 20 mins. The acetylation reaction was monitored by ninhydrin test; if it showed colorless, the coupling was completed. The resin was then washed with DMF (60 mL)*5, MeOH (20 mL)*5, and then dried under vacuum to afford peptide resin Intermediate 2 (SEQ ID NO:42) (CTC resin, 0.2 mmol, 1.9 g).

### Peptide Cleavage and Purification:

1) Cleavage buffer (95% TFA/2.5% TIS/2.5% H₂O, 20 mL) was added to the flask containing the side chain protected peptide resin (Intermediate 2, 1.9 g, SEQ ID NO:42) at room temperature and stirred for 2 hrs.
2) The filtrate was collected.
3) The peptide was precipitated with cold isopropyl ether (100 mL) and centrifuged (3 mins at 3000 rpm).
4) The peptide was washed with isopropyl ether two additional times, and the crude peptide was dried under vacuum for 2 hrs. to afford Compound 103 (SEQ ID NO:2) (1.23 g, crude) as a white solid.
5) The crude was purified by prep-HPLC (acidic conditions, TFA), followed by lyophilization to afford Cmp. 103 (SEQ ID NO:2) (32.8 mg, 91.8% purity, 3.17% yield) as a white solid. LCMS: RT=1.15 min, MS cal.: 4742.49, [2M/5+H]⁺=1897.64, [M/3+H]⁺=1581.47, [M/4+H]⁺=1186.47, [M/5+H]⁺=949.23.

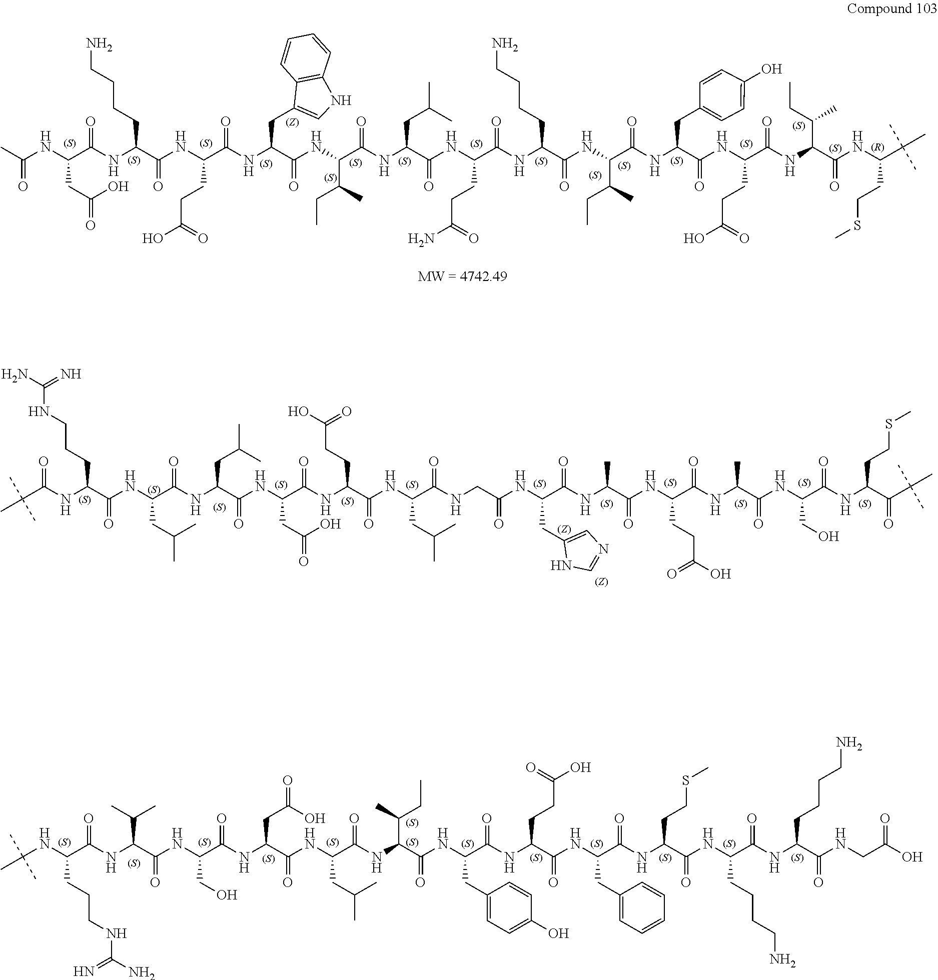

Compounds 104 and 105 were synthesized using the same procedure of peptide synthesis as for compounds 101-103, standard Fmoc chemistry, peptide cleavage and purification to afford the linear peptide.

0.5 mmol resin afforded compound 104 (SEQ ID NO:3), obtained (47.8 mg, 87.5% purity, 1.08% yield) as a white solid. LCMS: RT=1.13 min, MS cal.: 7736.79, [M/6+H]⁺=1290.52, [M/7+H]⁺=1106.14, [M/8+H]⁺=967.60.

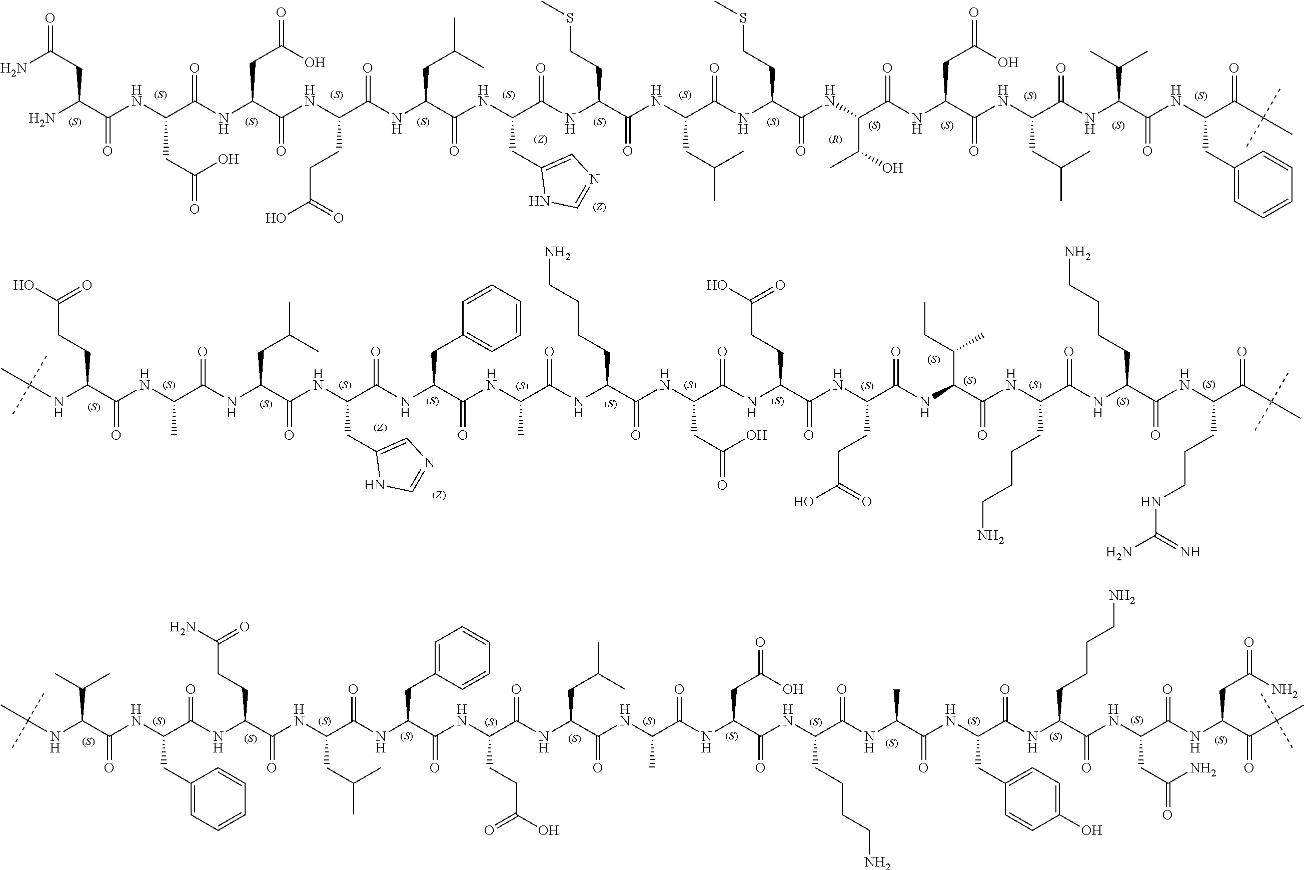

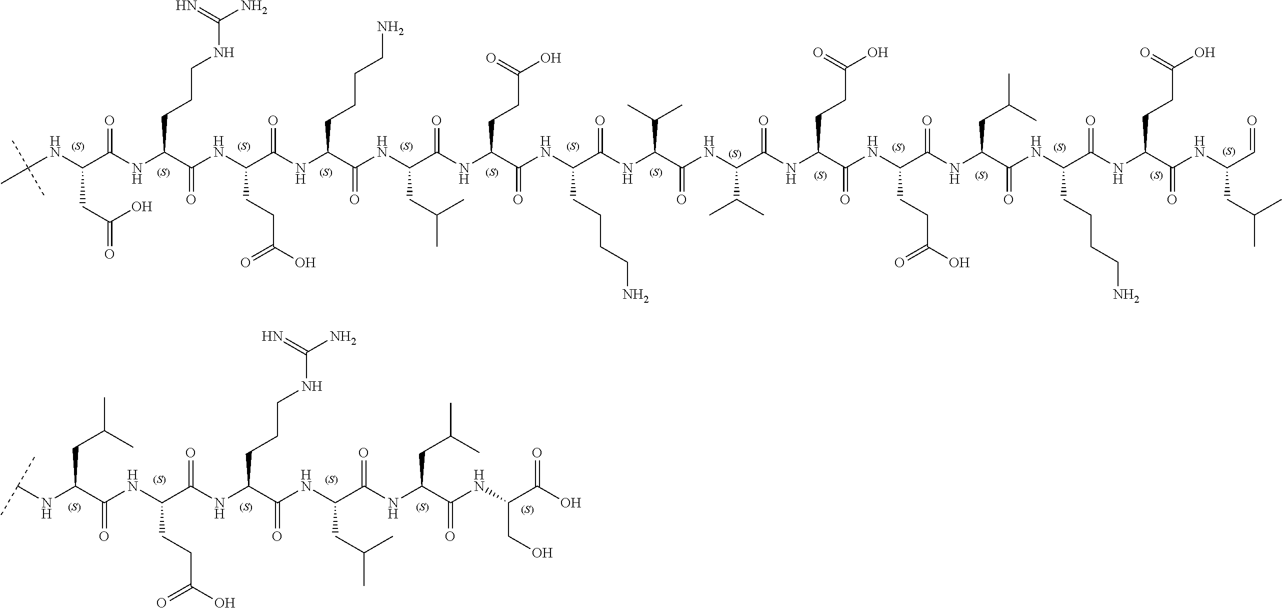

(Cmp. 104)
(SEQ ID NO: 3)
H2N-NDDELHMLMTDLVYEALHFAKDEEIKKRVFQLFELADKAYKNNDRQ
KLEKVVEELKELLERLLS-CO2

0.5 mmol resin afforded compound 105 (SEQ ID NO:4), obtained (40.5 mg, 95.0% purity, 1.08% yield) as a white solid. LCMS: RT=1.21 min, MS cal.: 6851.79, [M/5+H]⁺=1371.45, [M/6+H]⁺=1143.05, [M/7+H]⁺=979.59.

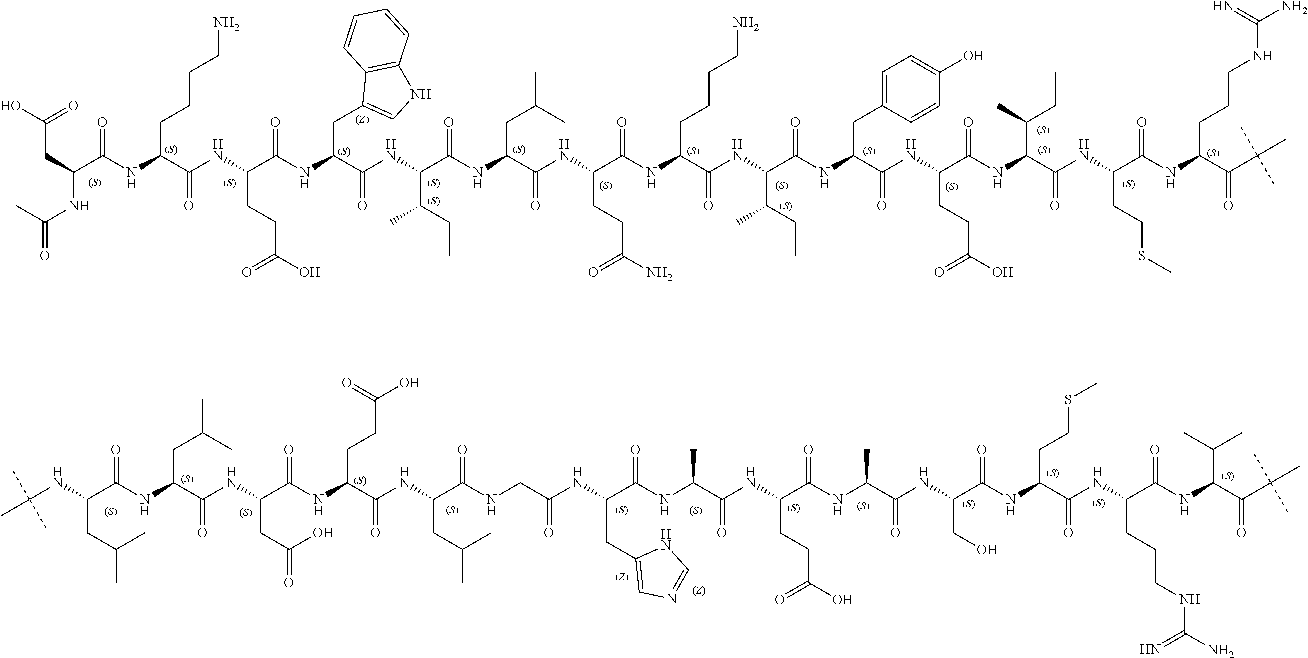

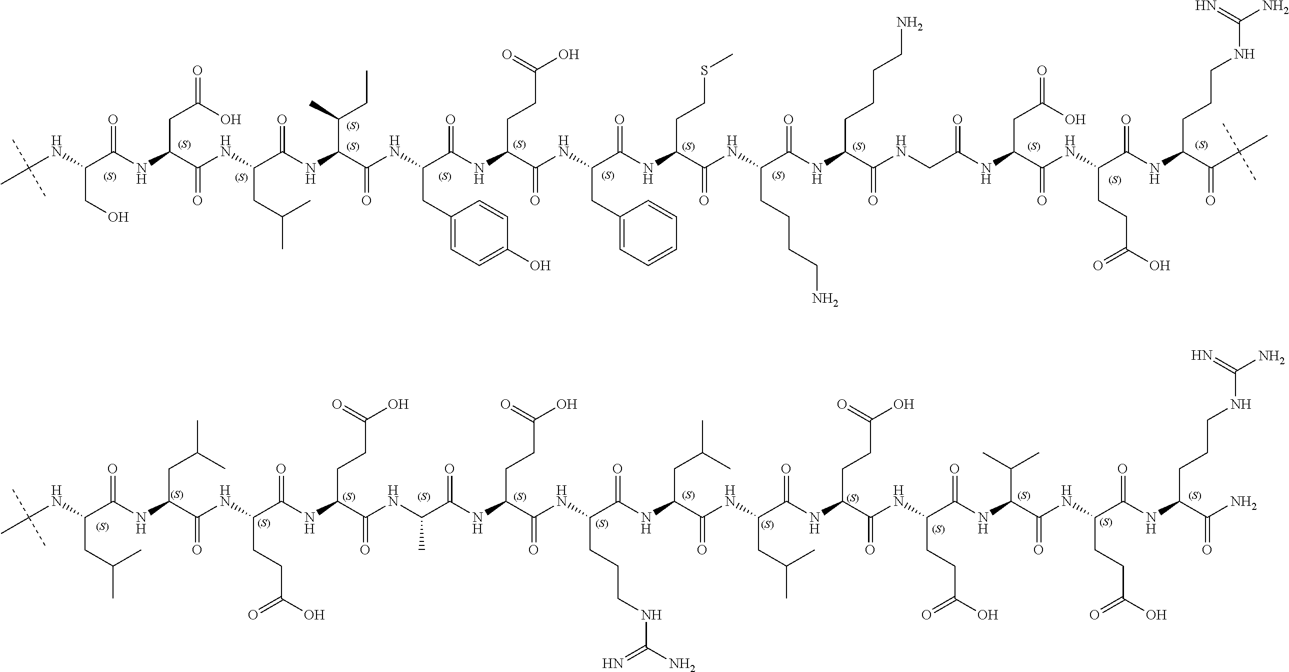

(Cmp. 105)
(SEQ ID NO: 4)
Ace-DKEWILQKIYEIMRLLDELGHAEASMRVSDLIYEFMKKGDERLLEE
AERLLEEVER-NH2

### Example 3. Procedure for Preparation of Compound 106 (Single SPPS to Form Disulfide Peptide)

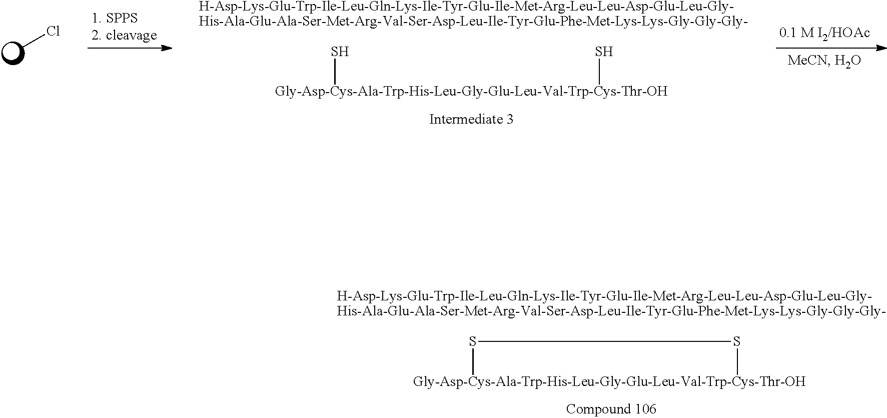

### Preparation of Intermediate 3

Intermediate 3 (SEQ ID NO:5) was synthesized using standard peptide synthesis Fmoc chemistry and peptide cleavage (see synthesis of cmpd. 102, Example 1). 0.5 mmol resin afforded Intermediate 3 (SEQ ID NO:5) (3.0 g, crude) as a white solid.

### Preparation of Compound 106:

To a mixture of Intermediate 3 (SEQ ID NO:5) (3.0 g, crude) in ACN/H₂O (3:7, 500 mL) was added 0.1 M I₂/AcOH dropwise until a yellow color persisted, then the mixture was stirred at 20° C. for 5 mins. The mixture was quenched with 0.1 M Na₂S₂O₃ dropwise until the yellow color disappeared. After filtration, the filtrate was purified by prep-HPLC (acidic conditions, TFA). Some less pure fractions were re-purified by prep-HPLC (NH₄OAc conditions), followed by lyophilization to afford Compound 106 (SEQ ID NO:6) (Batch1: 47.9 mg, 84.1% purity, TFA salt; Batch2: 15.2 mg, 97.2% purity, NH₄OAc salt) as a white solid. LCMS: RT=1.05 min, MS cal.: 6384.32, [M/4+H]⁺=196.70, [M/5+H]⁺=1277.81, [M/6+H]⁺=1264.90.

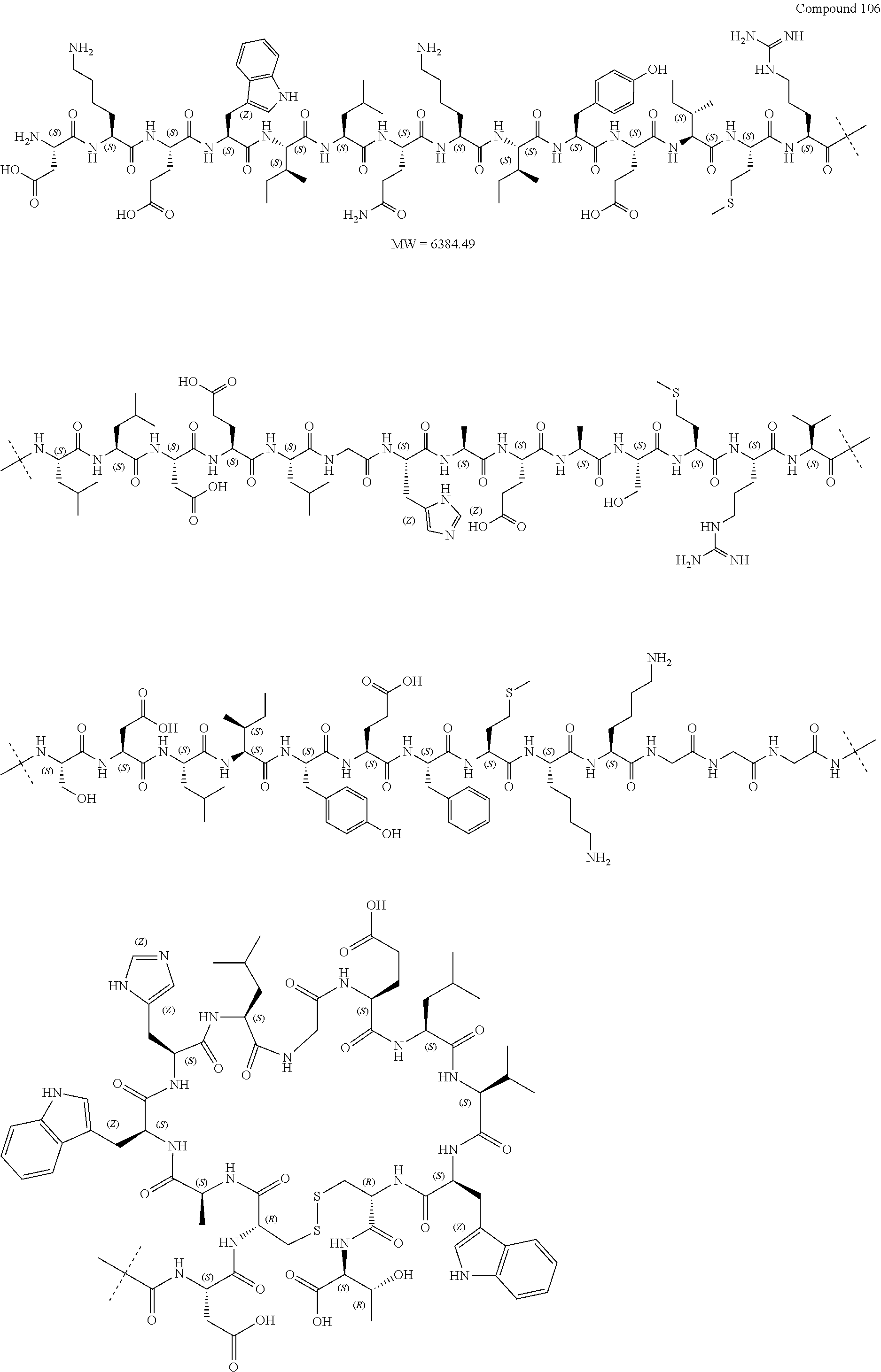

Disulfide peptides, Compounds 107, 108, 109, 110, 111, 112, 113, 114, 115, 116, 117, 118, 119, 120, 121, 122, and 123, were synthesized using the same procedure as Compound 106.

0.5 mmol resin afforded Compound 107 (Batch1: 30.0 mg, 81.6% purity, TFA salt; batch2: 10.4 mg, 96.0% purity, NH₄OAc salt) as a white solid. LCMS: RT=1.05 min, MS cal.: 6426.36, [M/4+H]⁺=1607.60, [M/5+H]⁺=1286.80, [M/6+H]⁺=1071.75.

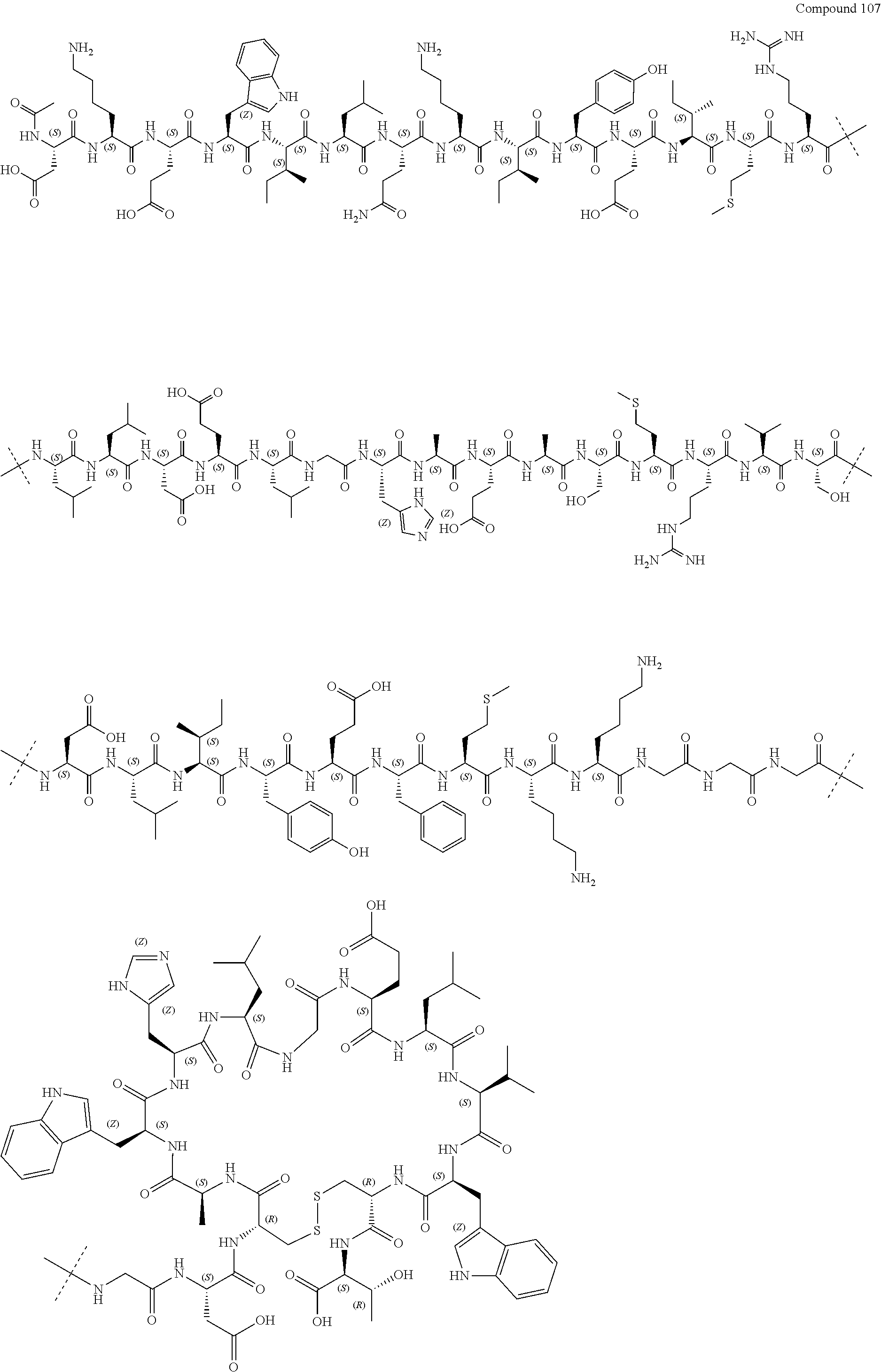

0.5 mmol resin afforded Compound 108 (batch 1: 24.0 mg, 85.7% purity, TFA salt; batch 2: 11.1 mg, 96.6% purity, NH₄OAc salt) as a white solid. LCMS: RT=1.05 min, MS cal.: 6509.49, [M/4+H]⁺=1628.19, [M/5+H]⁺=1302.98, [M/6+H]⁺=1085.58.

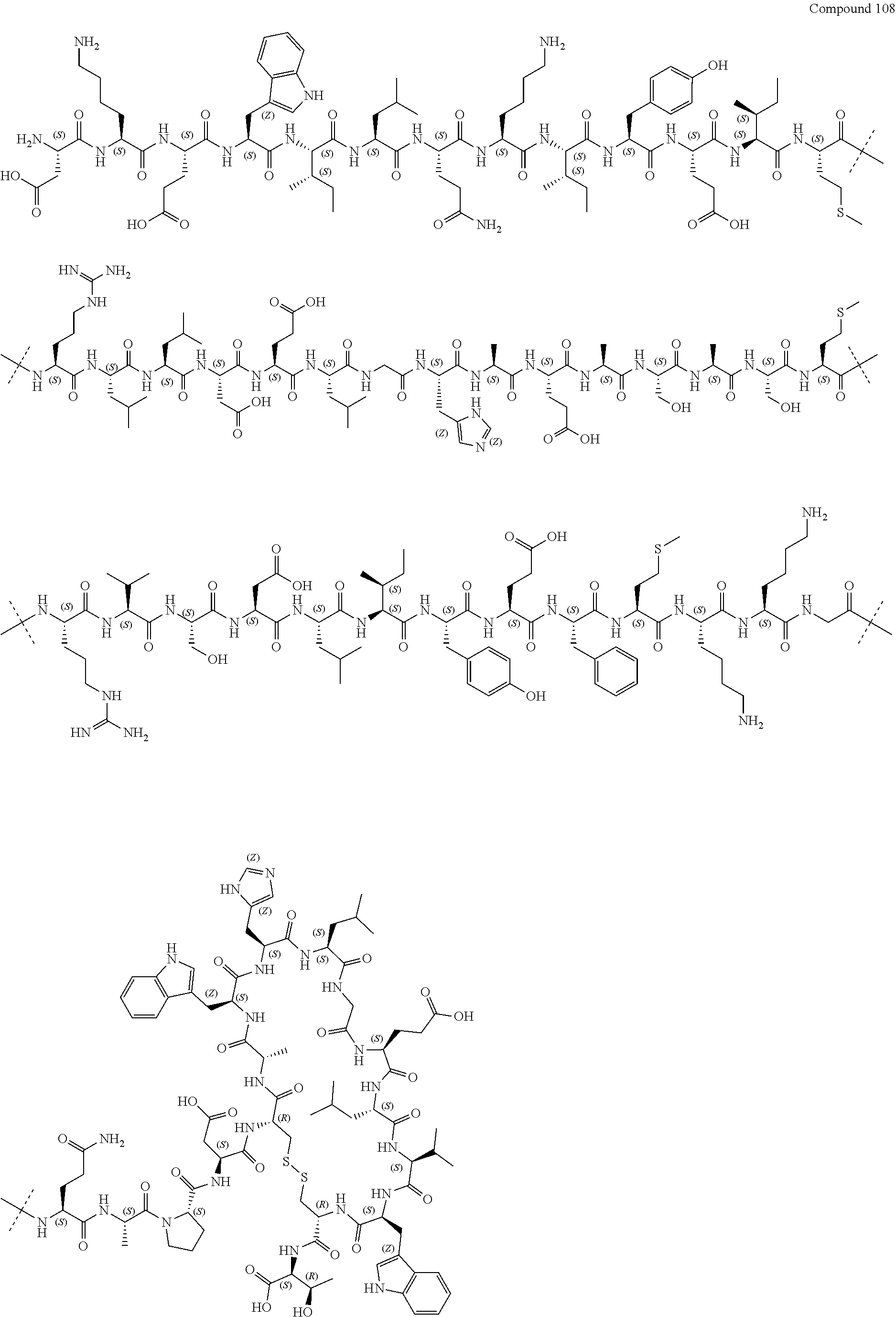

0.5 mmol resin afforded Compound 109 (Batch1: 38.5 mg, 85.9% purity, TFA salt; batch2: 38.0 mg, 90.6% purity, NH₄OAc salt) as a white solid. LCMS: RT=1.05 min, MS cal.: 6551.53, [M/4+H]⁺=1638.42, [M/5+H]⁺=1311.10, [M/6+H]⁺=1092.70.

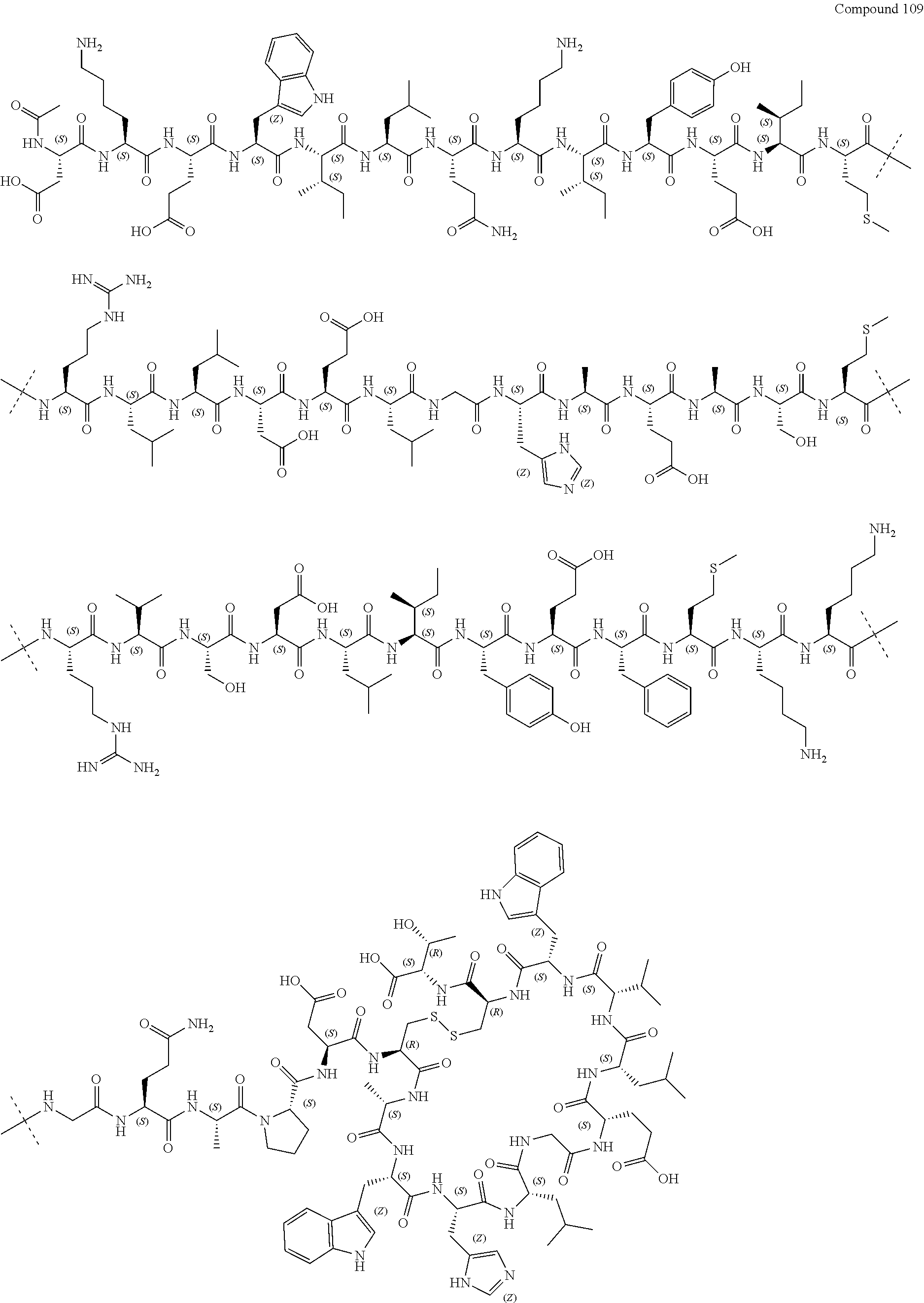

0.5 mmol resin afforded Compound 110 (batch 1: 14.5 mg, 88.1% purity; batch 2: 40.7 mg, 91.1% purity; batch 3: 21.8 mg, 93.4% purity) as a white solid. LCMS: RT=2.35 min, MS cal.: 8365.49, [M/5+H]⁺=1674.00, [M/6+H]⁺=1395.30, [M/7+H]⁺=1196.10.

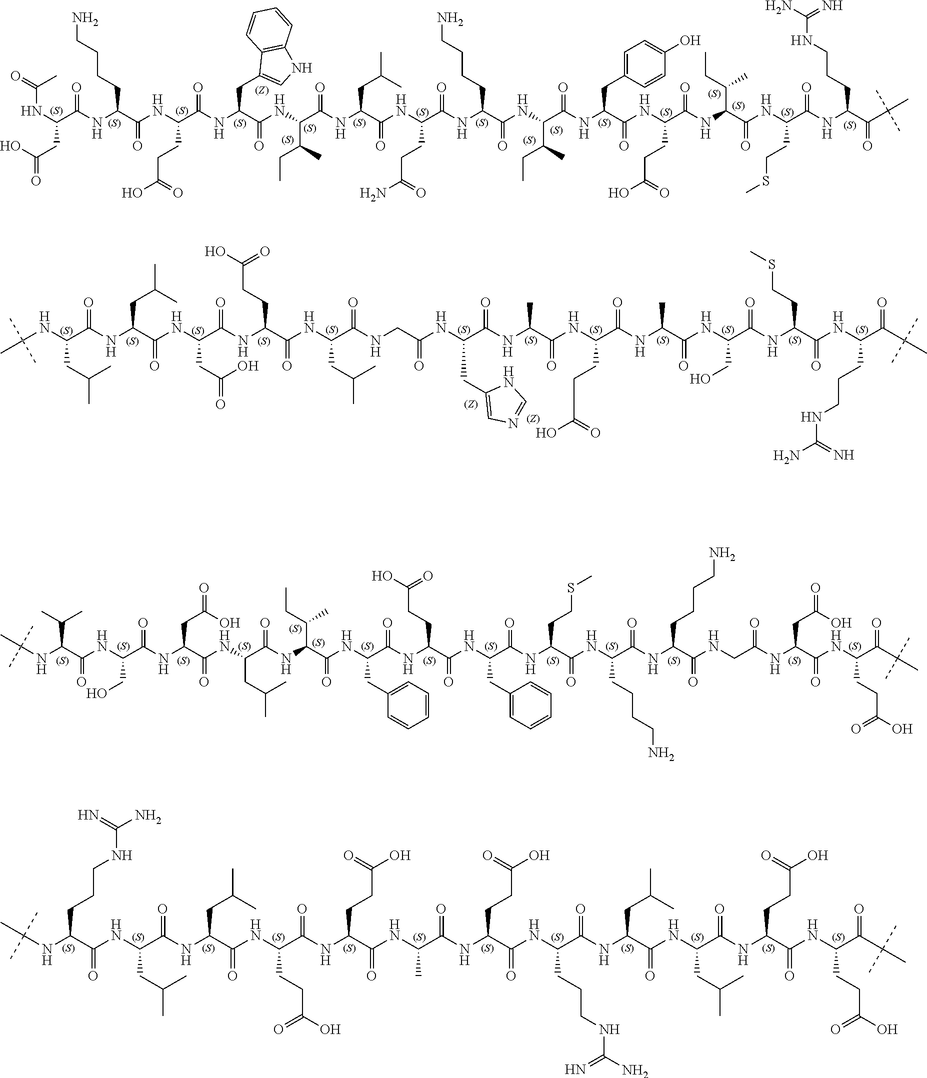

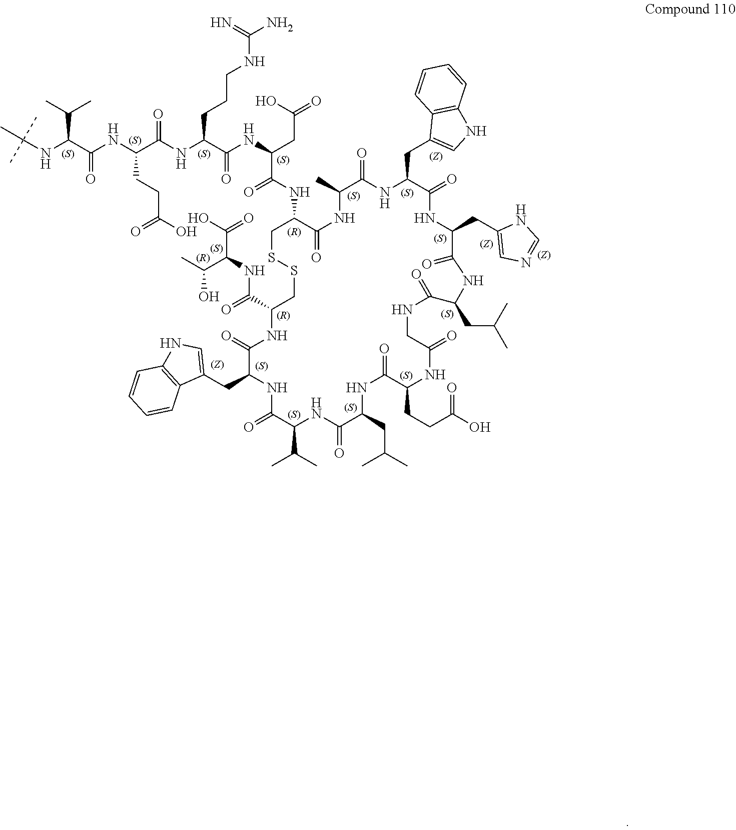

(SEQ ID NO: 18)
Ac-DKEWILQKIYEIMRLLDELGHAEASMRVSDLIYEFMKKGDERLLEEA
ERLLEEVER-D[CAWHLGELVWC]T-CO2

0.5 mmol resin afforded Compound 111 (batch 1: 43.5 mg, 94.1% purity; batch2: 15.8 mg, 87.0% purity) as a white solid. LCMS: RT=2.31 min, MS cal.: 8323.45, [M/5+H]⁺=1665.60, [M/6+H]⁺=1387.80, [M/7+H]⁺=1189.90.

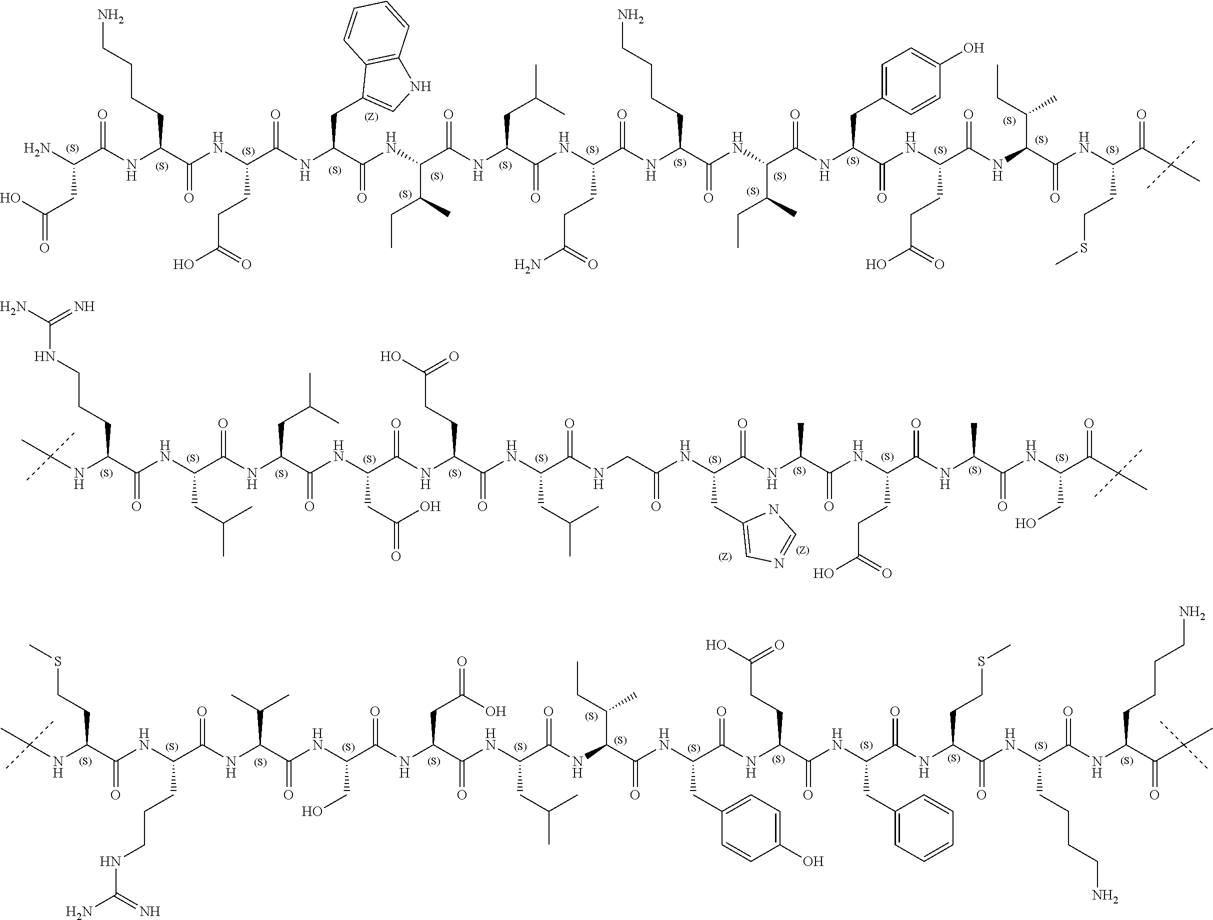

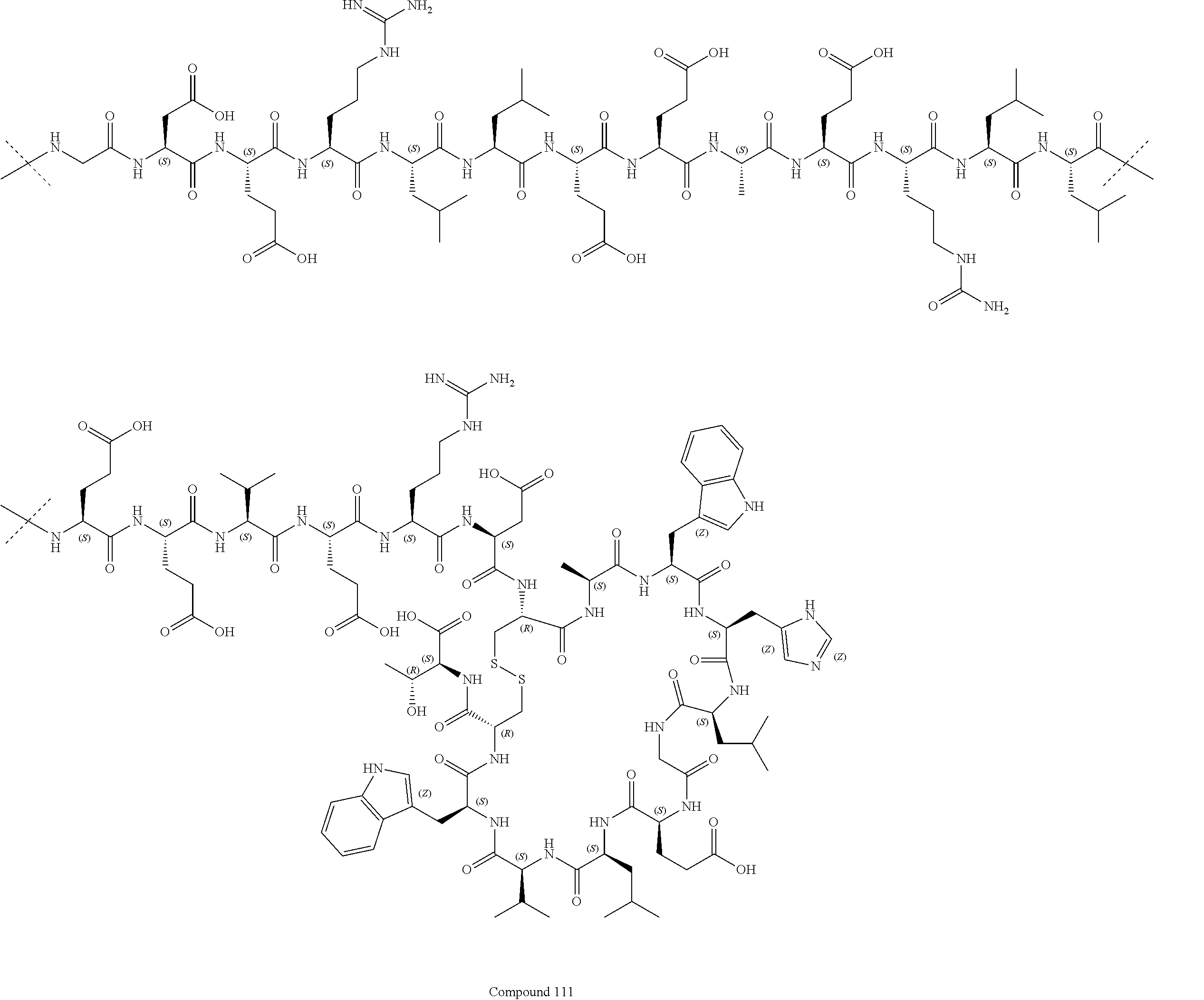

0.5 mmol resin afforded Compound 112 (16.4 mg, 91.2% purity) as a white solid. LCMS: RT=1.10 min, MS cal.: 6255.20, [M/4+H]⁺=1564.29, [M/5+H]⁺=1251.65.

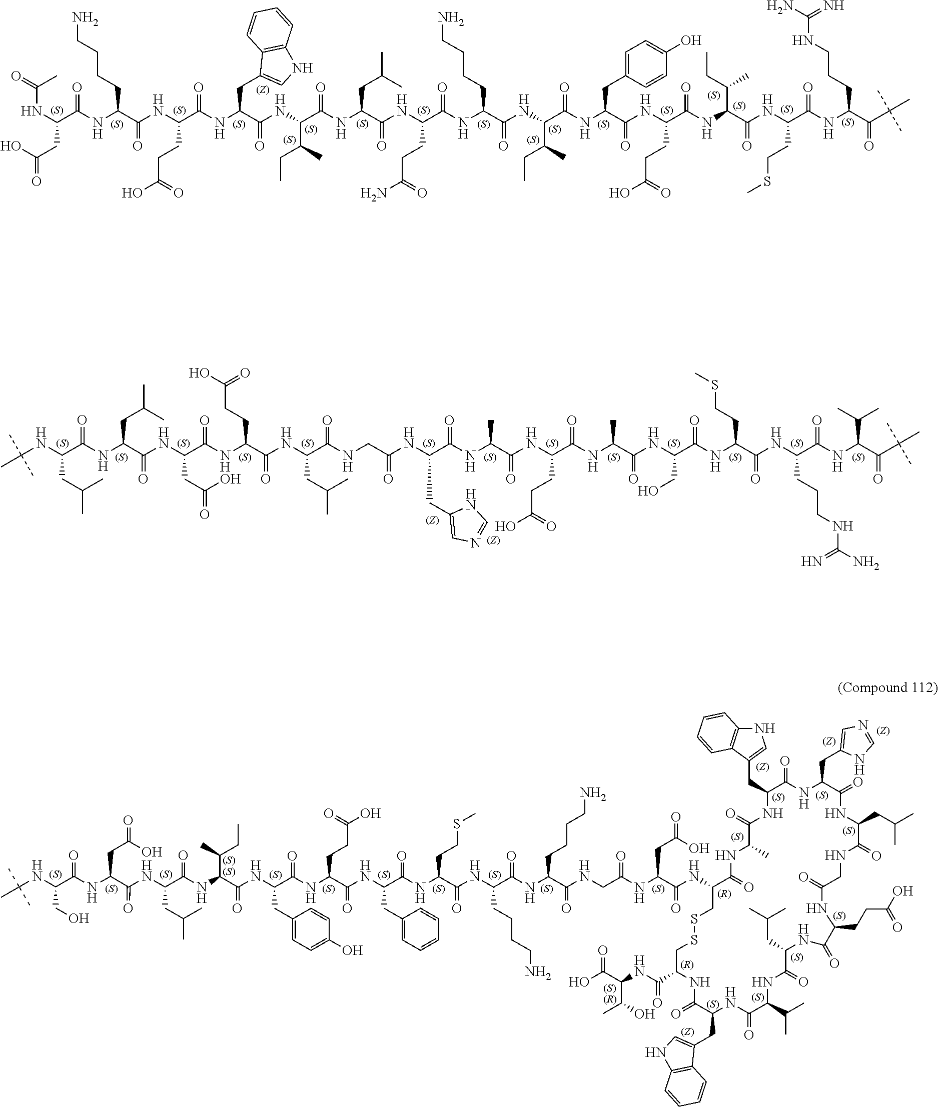

0.5 mmol resin afforded Compound 113 (36.2 mg, 85.0% purity) as a white solid. LCMS: RT=1.05 min, MS cal.: 6213.17, [M/4+H]⁺=1554.19.

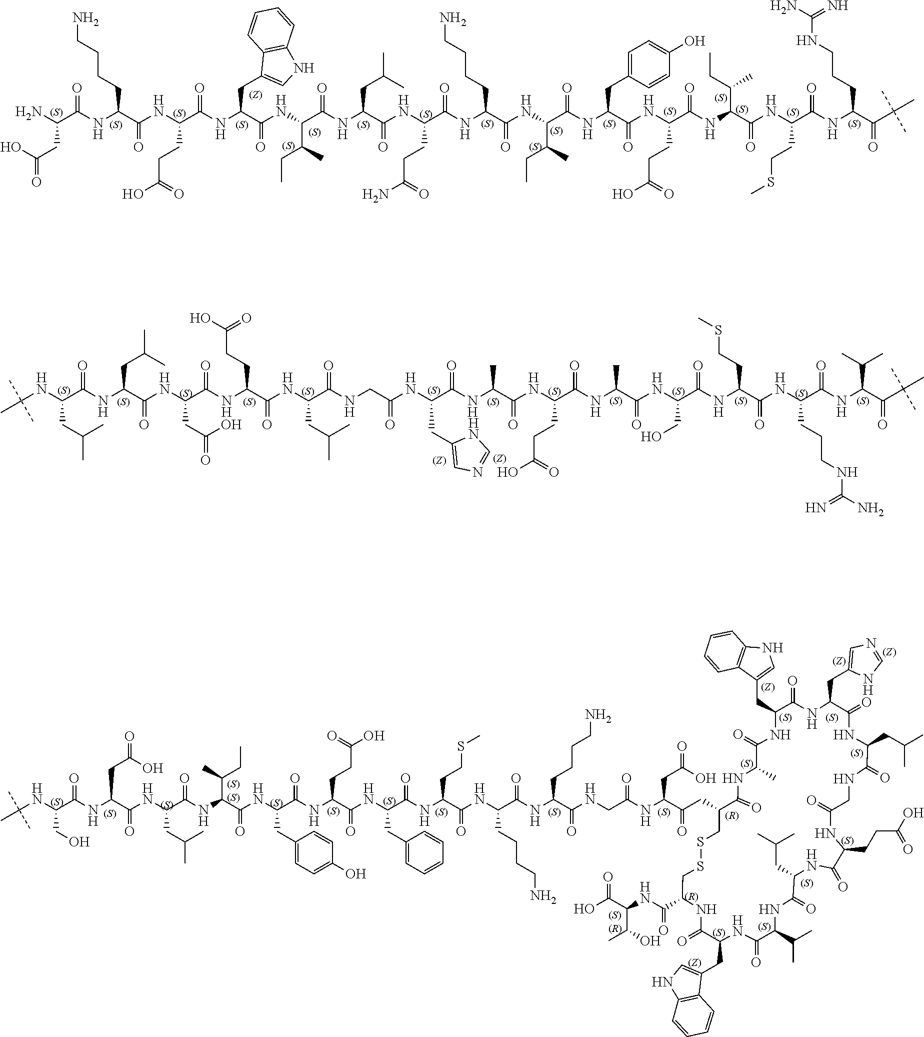

0.5 mmol resin afforded Compound 114 (46.1 mg, 91.9% purity) as a white solid. LCMS: RT=1.95 min, MS cal.: 6198.15, [M/4+H]⁺=1551.20, [M/5+H]⁺=1241.10, [M/6+H]⁺=1034.50.

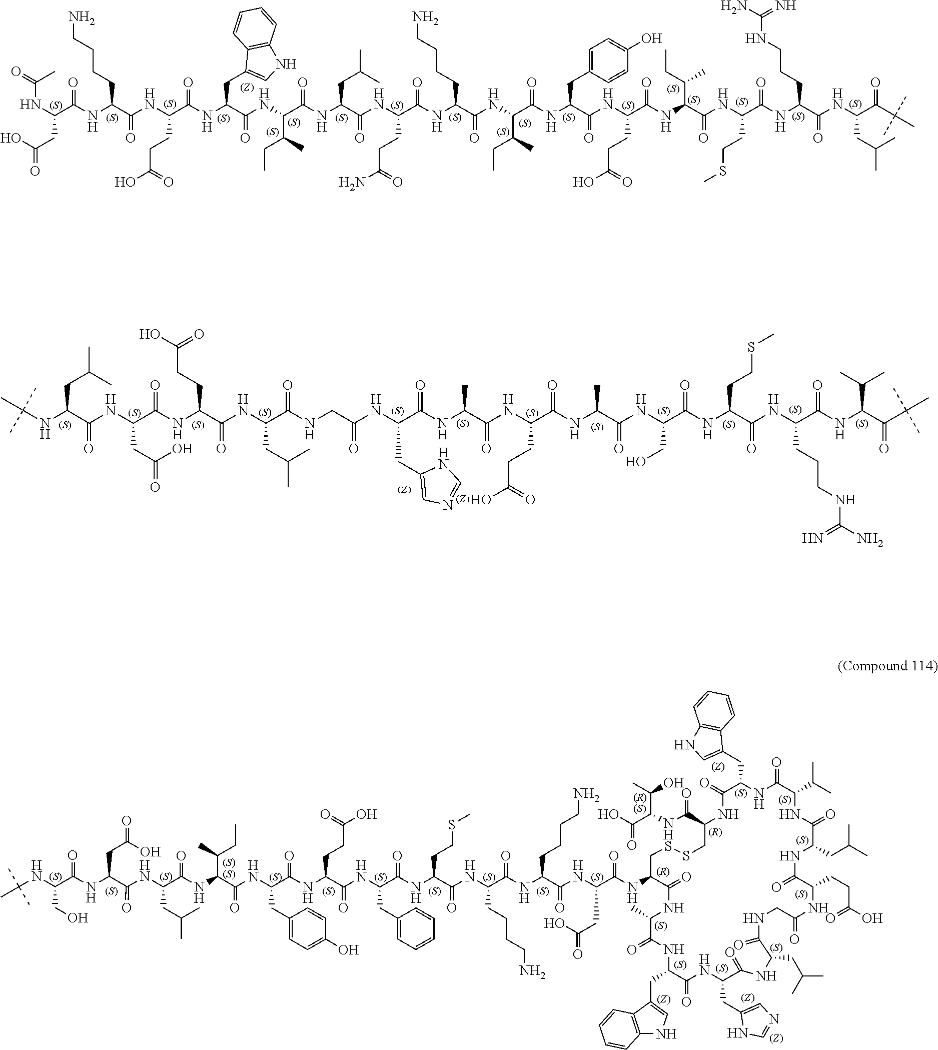

0.5 mmol resin afforded Compound 115 (51.5 mg, 85.1% purity) as a white solid. LCMS: RT=1.02 min, MS cal.: 6156.12, [M/4+H]⁺=1539.81, [M/5+H]⁺=1232.09.

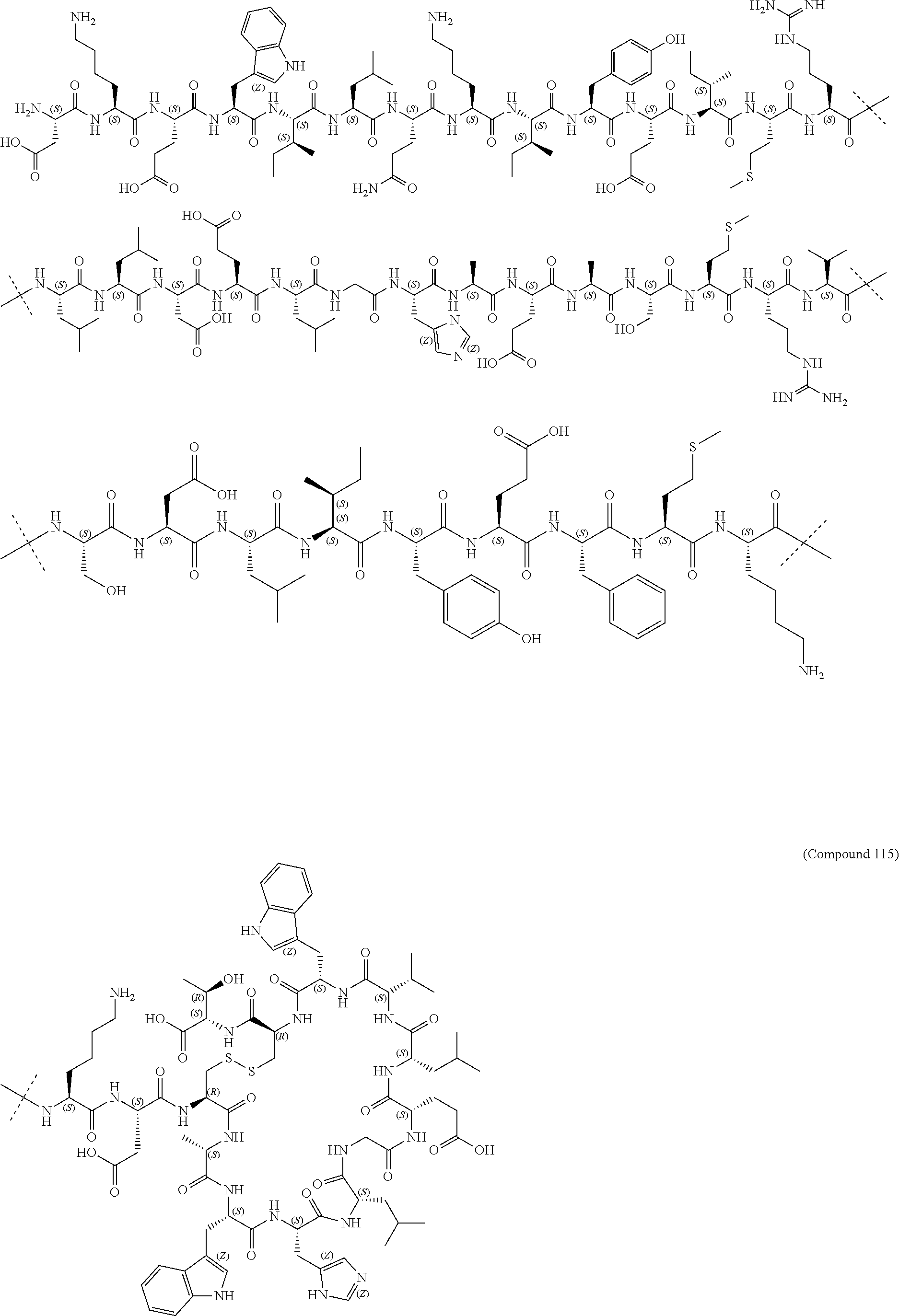

0.5 mmol resin afforded Compound 116 (1.3 mg, 90.0% purity) as a white solid. LCMS: RT=2.36 min, MS cal.: 5696.62, [M/3+H]⁺=1899.3, [M/4+H]⁺=1424.7, [M/5+H]⁺=1141.0.

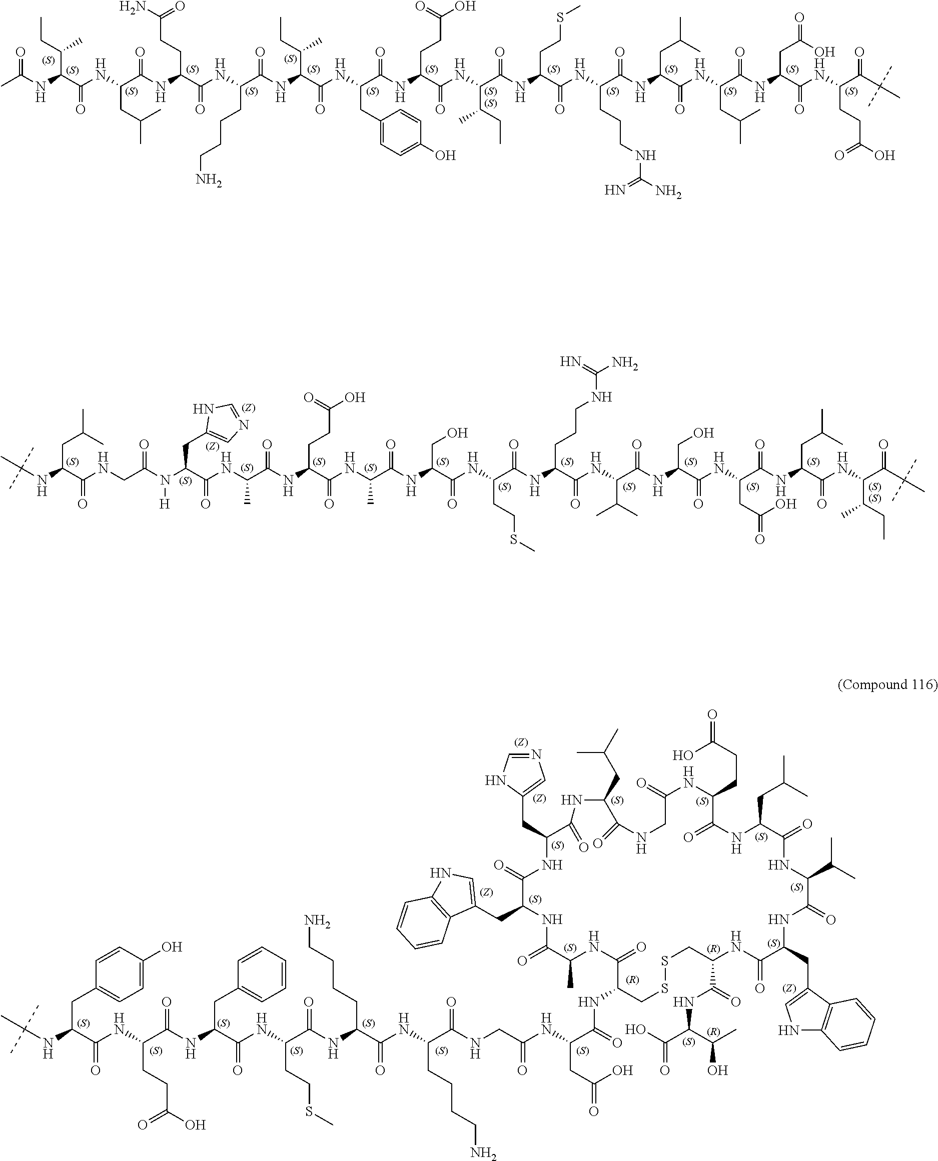

0.5 mmol resin afforded Compound 117 obtains (Batch1: 44.0 mg, 86.5% purity, TFA salt; batch2: 8.5 mg, 90% purity, NH₄OAc salt) as a white solid. LCMS: RT=2.36 min, MS cal.: 5696.62, [M/3+H]⁺=1899.3, [M/4+H]⁺=1424.7, [M/5+H]⁺=1141.0.

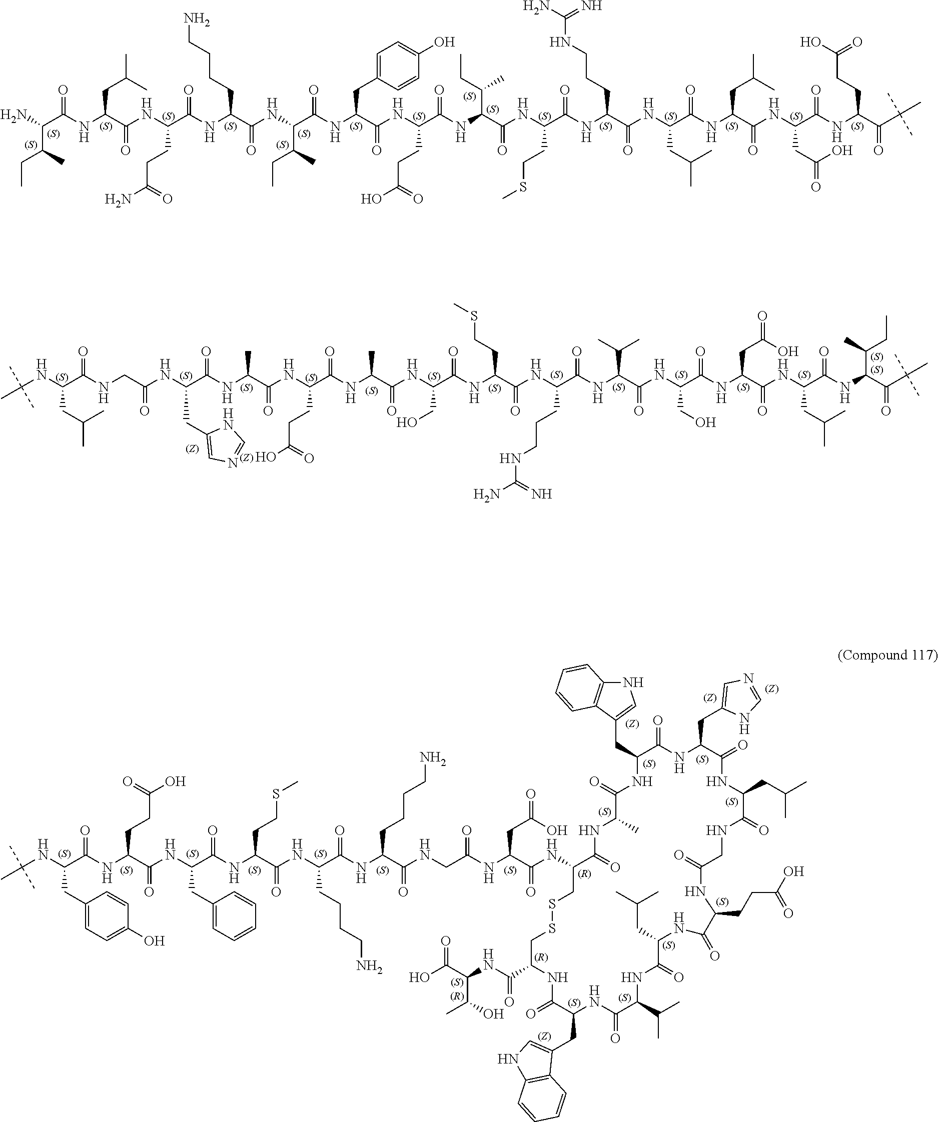

0.5 mmol resin afforded Compound 118 (24.8 mg, 91.1% purity) as a white solid. LCMS: RT=1.12 min, MS cal.: 5639.57, [M/4+H]⁺=1410.68, [M/5+H]⁺=1128.83, [M/6+H]⁺=941.00.

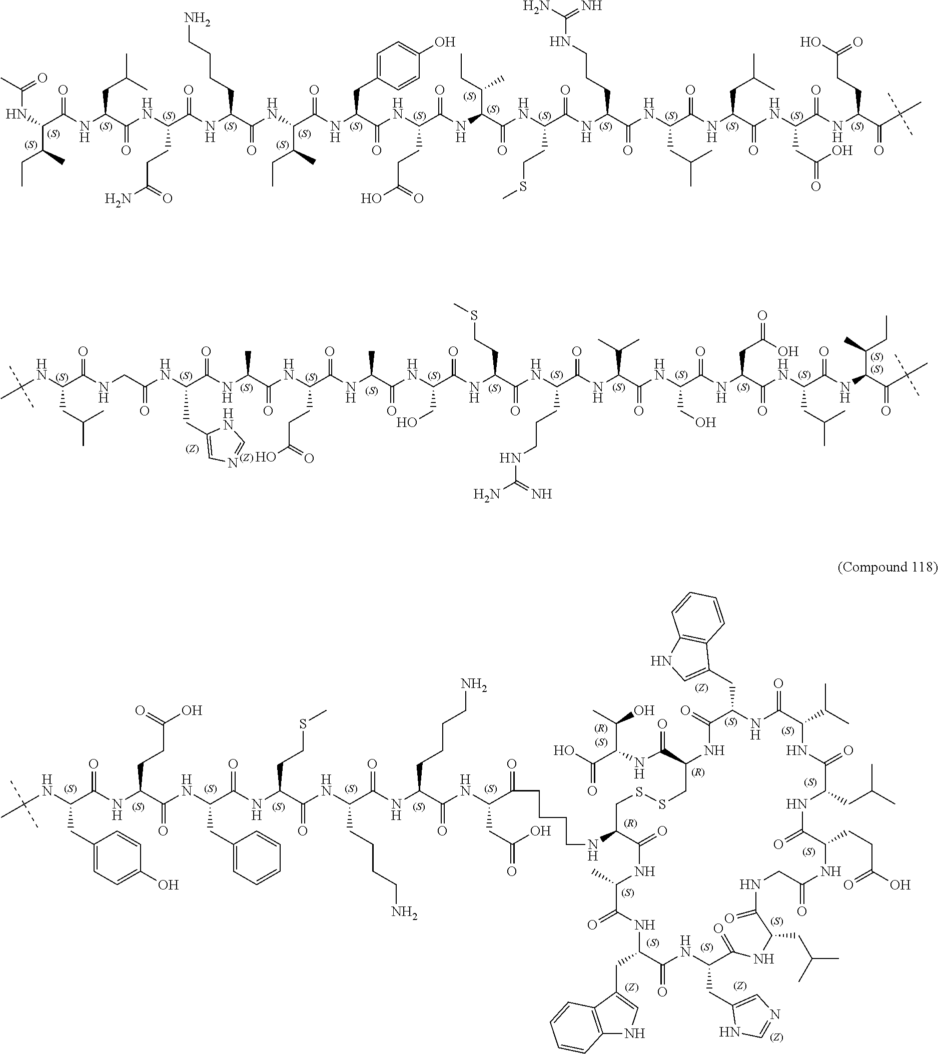

0.5 mmol resin afforded Compound 119 (30.3 mg, 90.1% purity) as a white solid. LCMS: RT=1.84 min, MS cal.: 5597.53, [M/3+H]⁺=1867.20, [M/4+H]⁺=1400.90, [M/5+H]⁺=1120.90.

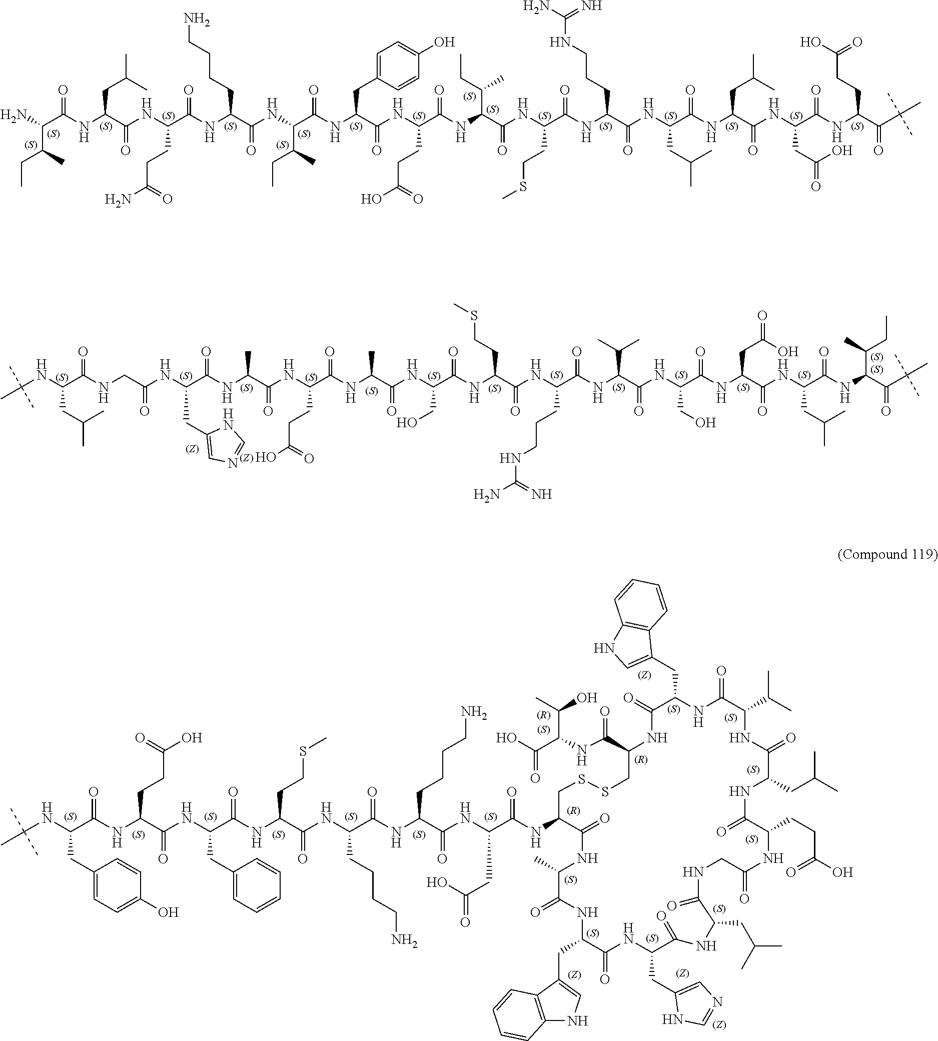

0.5 mmol resin afforded Compound 120 (Batch1: 28.4 mg, 93.0% purity; batch2: 24.5 mg, 94.7% purity) as a white solid. LCMS: RT=2.35 min, MS cal.: 8365.49, [M/5+H]⁺=1573.80, [M/6+H]=1395.30, [M/7+H]⁺=1195.90.

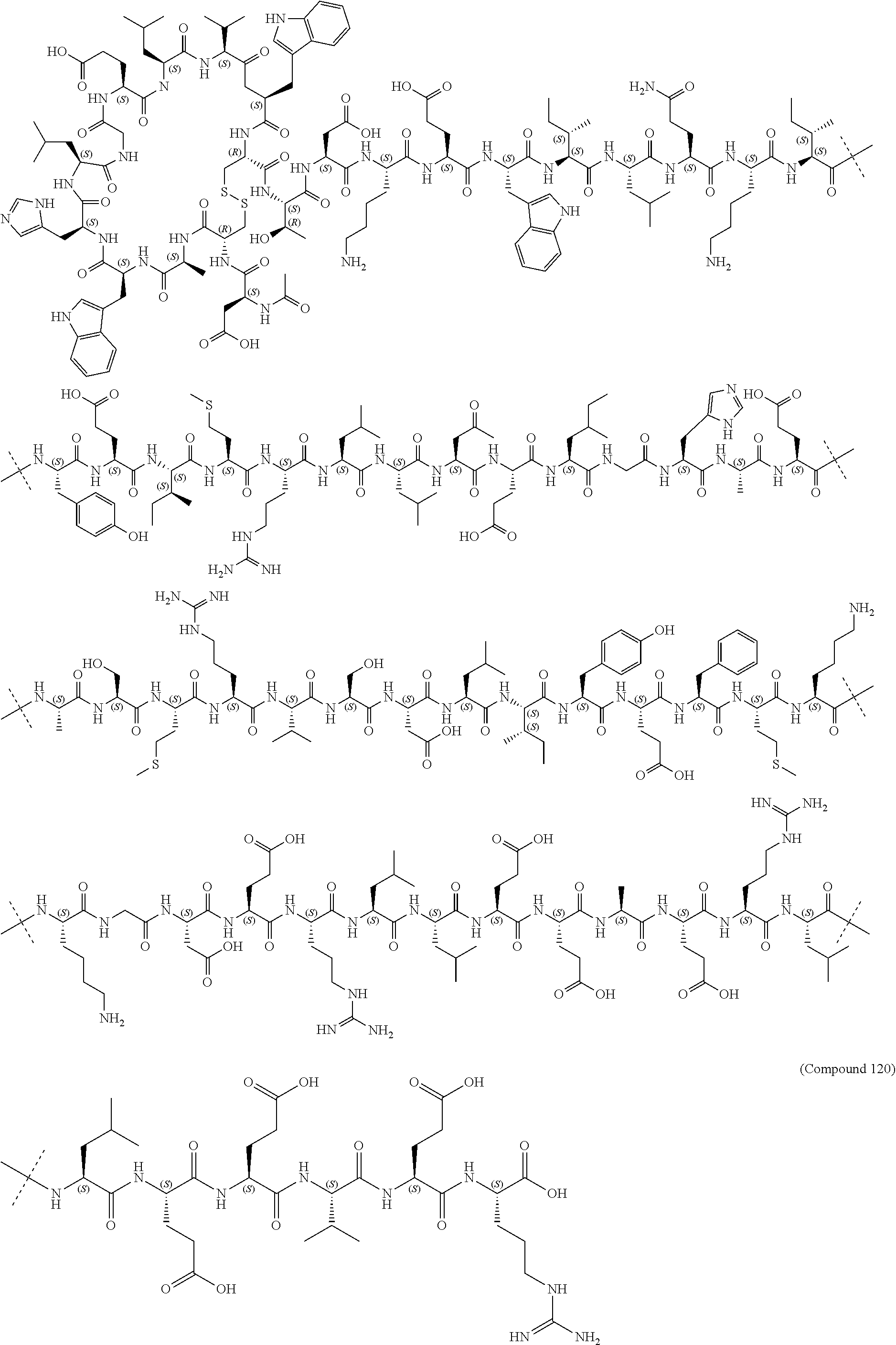

(SEQ ID NO: 7)
uABT-DCAWILQKIYEIMRLLDELGHAEASMRVSDLIYEFMKKGDERLLE
EAERLLEEVER-CO2

0.5 mmol resin afforded Compound 121 (10.1 mg, 85.5% purity) as a white solid. LCMS: RT=2.24 min, MS cal.: 6198.15, [M/4+H]⁺=1549.90, [M/5+H]⁺=1240.40, [M/6+H]⁺=1033.90.

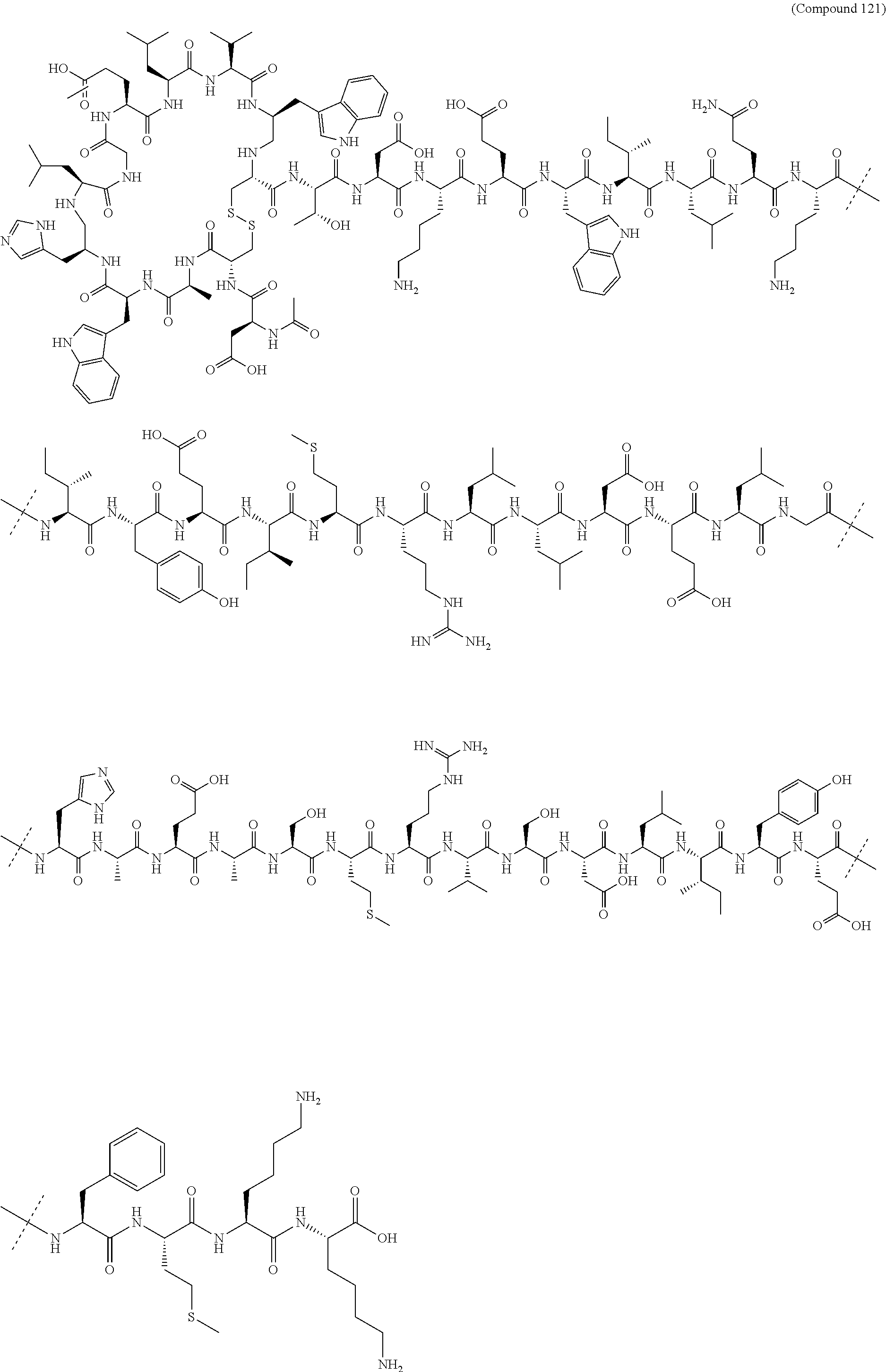

0.5 mmol resin afforded Compound 122 (Batch1: 9.6 mg, 90.2% purity; batch2: 8.8 mg, 91.2% purity) as a white solid. LCMS: RT=2.45 min, MS cal.: 5639.57, [M/3+H]⁺=1880.60, [M/4+H]⁺=1410.70, [M/5+H]⁺=1128.70.

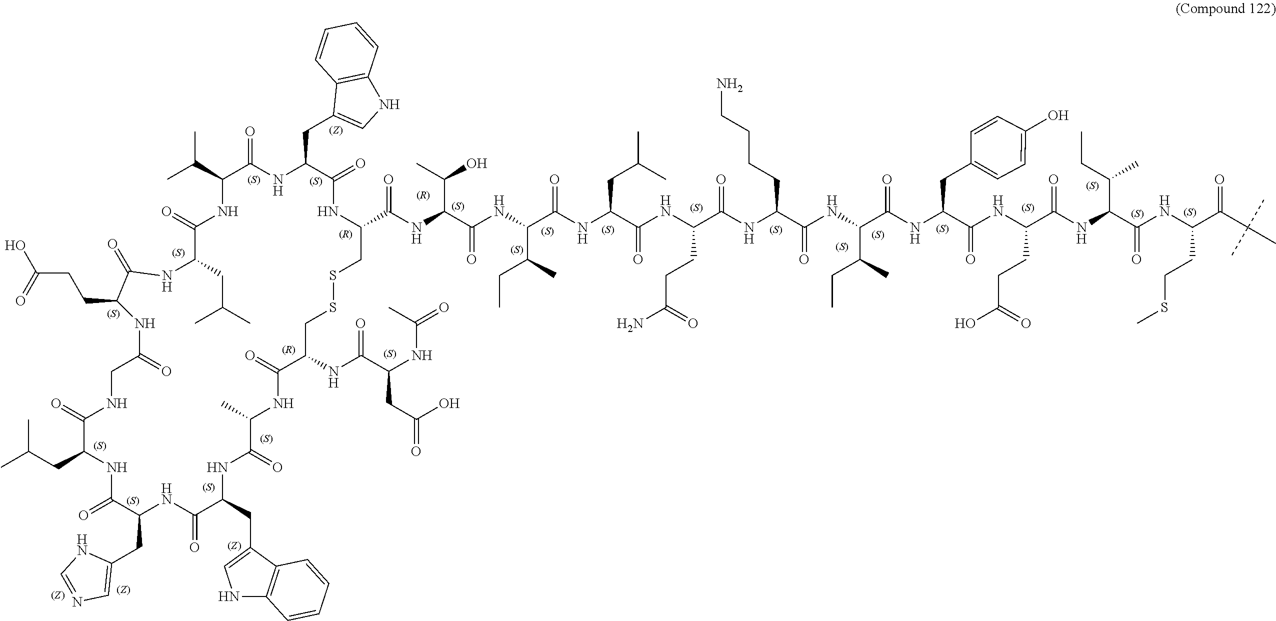

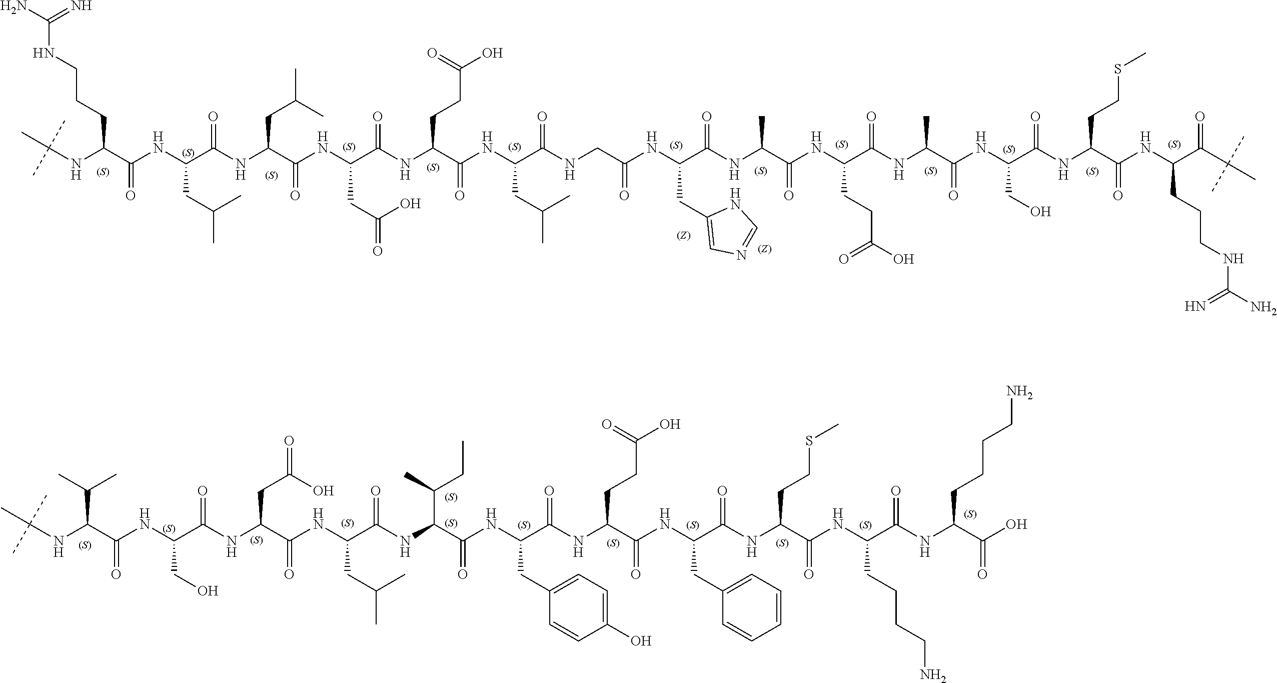

0.5 mmol resin afforded Compound 123 (batch1: 24.1 mg, 82.6% purity, TFA salt; batch2: 27.8 mg, 90.5% purity, NH₄OAc salt) as a white solid. LCMS: RT=1.10 min, MS cal.: 9169.46, [M/5+H]⁺=1834.85, [M/7+H]⁺=1310.71, [M/8+H]⁺=1146.91.

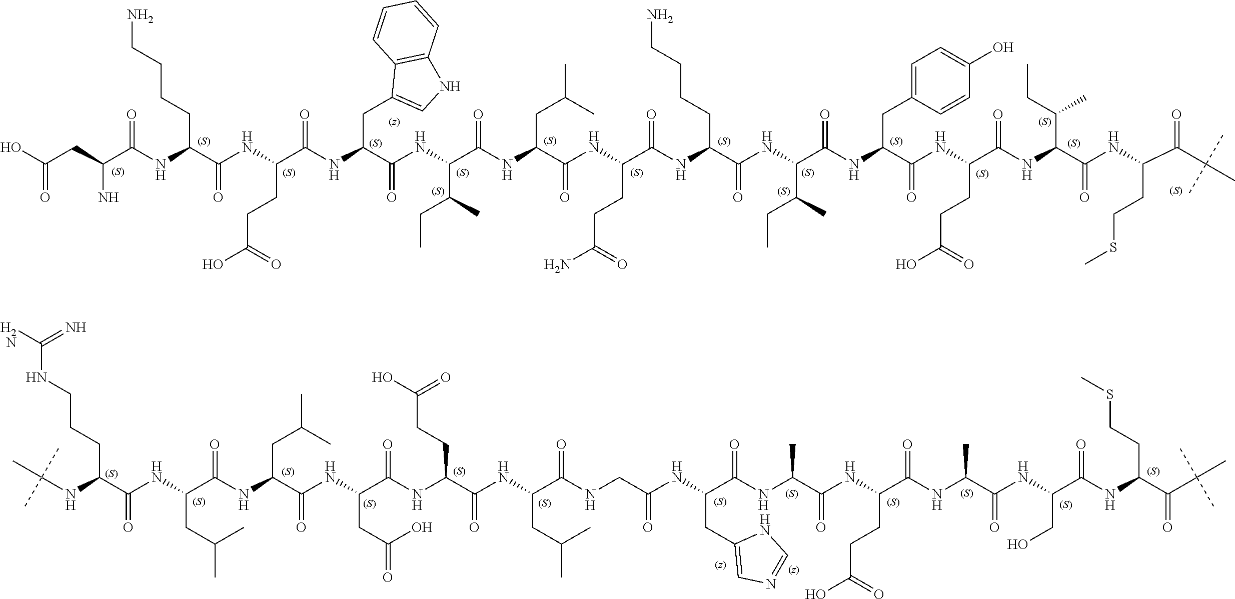

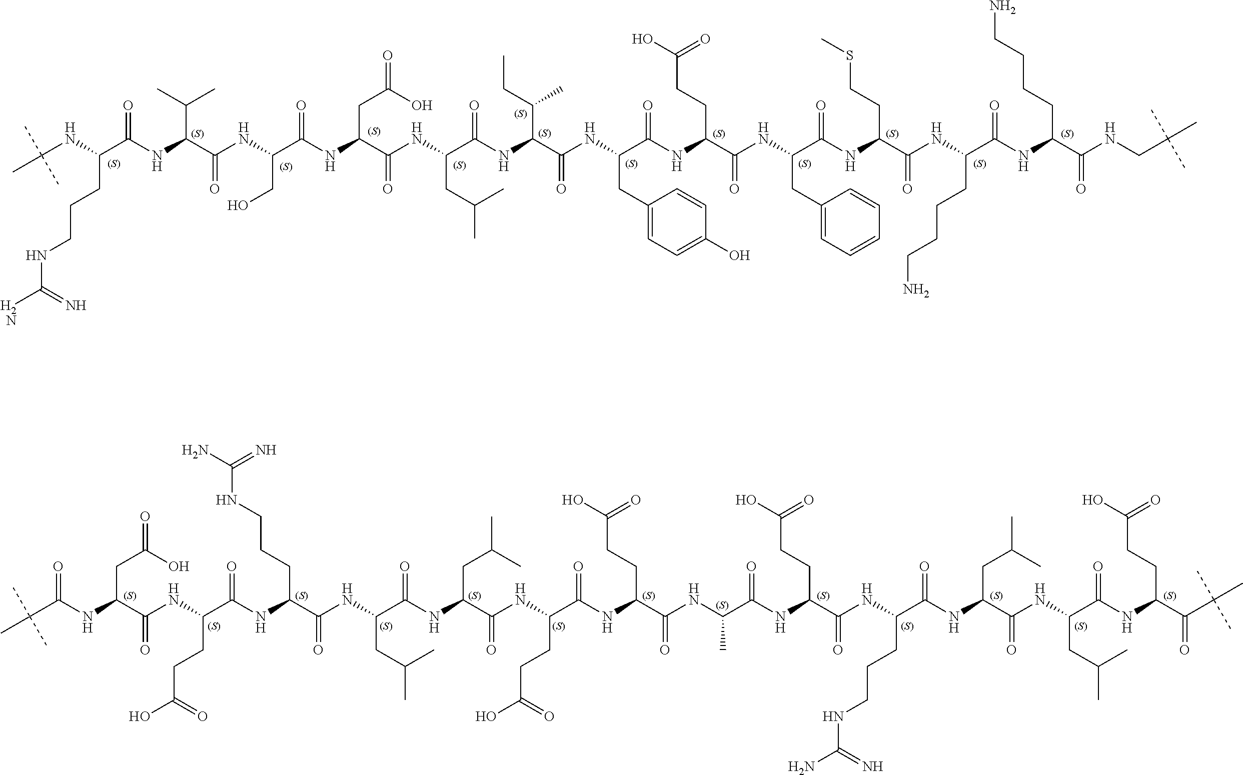

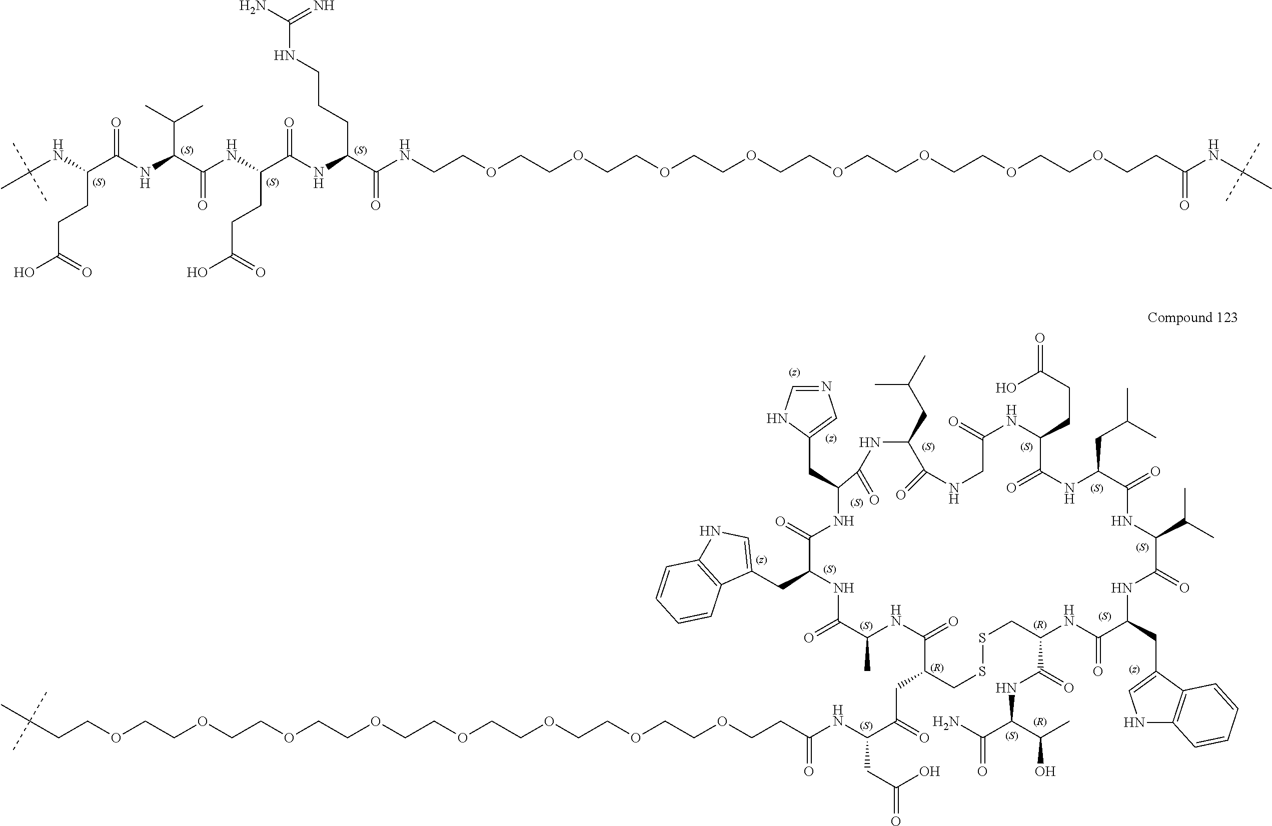

(SEQ ID NO: 8)
Ace-DKEWILQKIYEIMRLLDELGHAEASMRVSDLIYEFMKKGDERLLEE
AERLLEEVER-Peg8-Peg8-uABT

0.5 mmol resin afforded Compound 124 (47.9 mg, 92.5% purity, NH₄OAc salt) as a white solid. LCMS: RT=1.12 min, MS cal.: 9211.50, [M/5+H]⁺=1842.94, [M/7+H]⁺=1316.80, [M/8+H]⁺=1152.15.

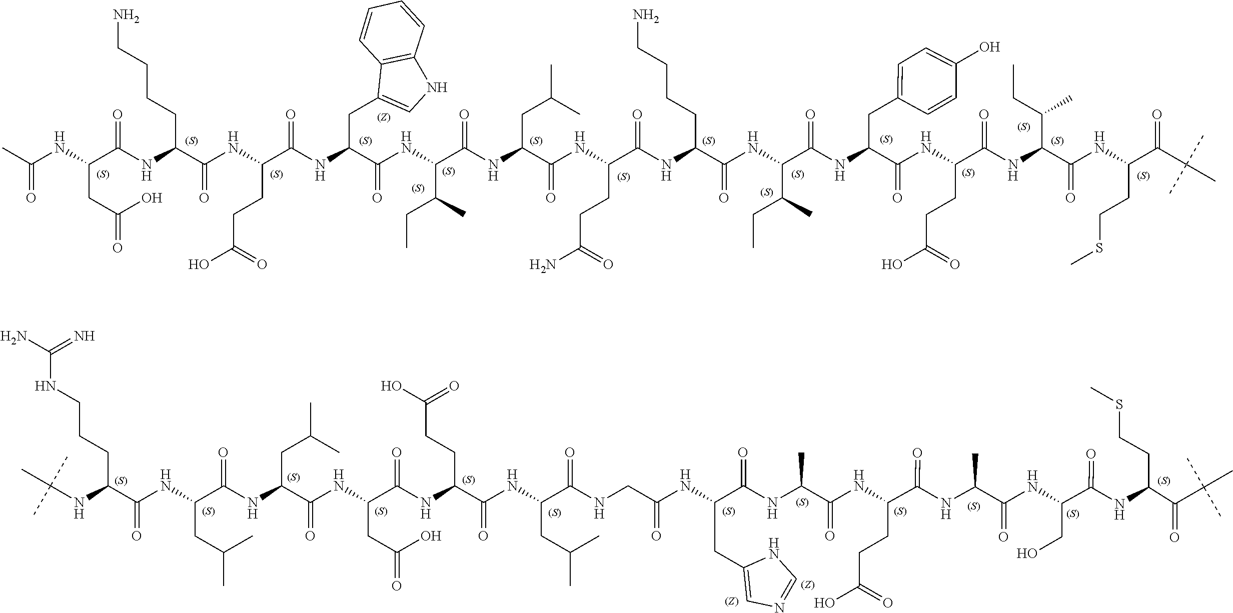

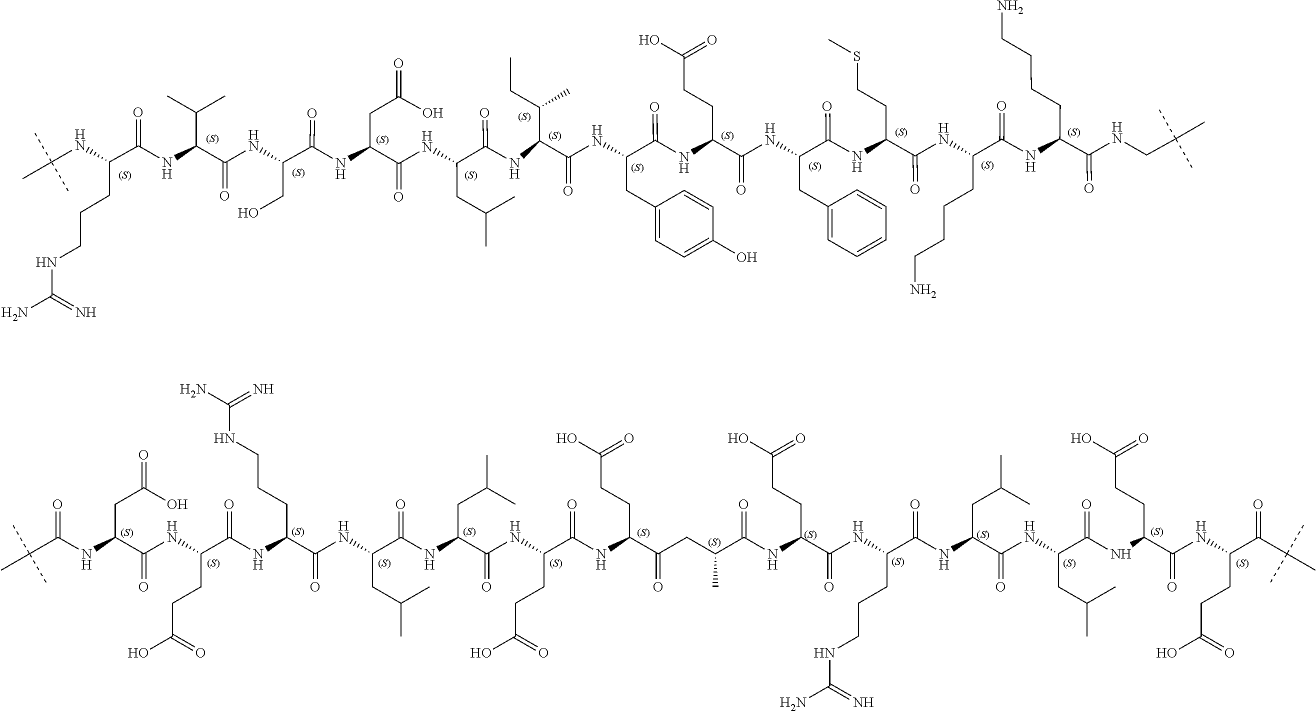

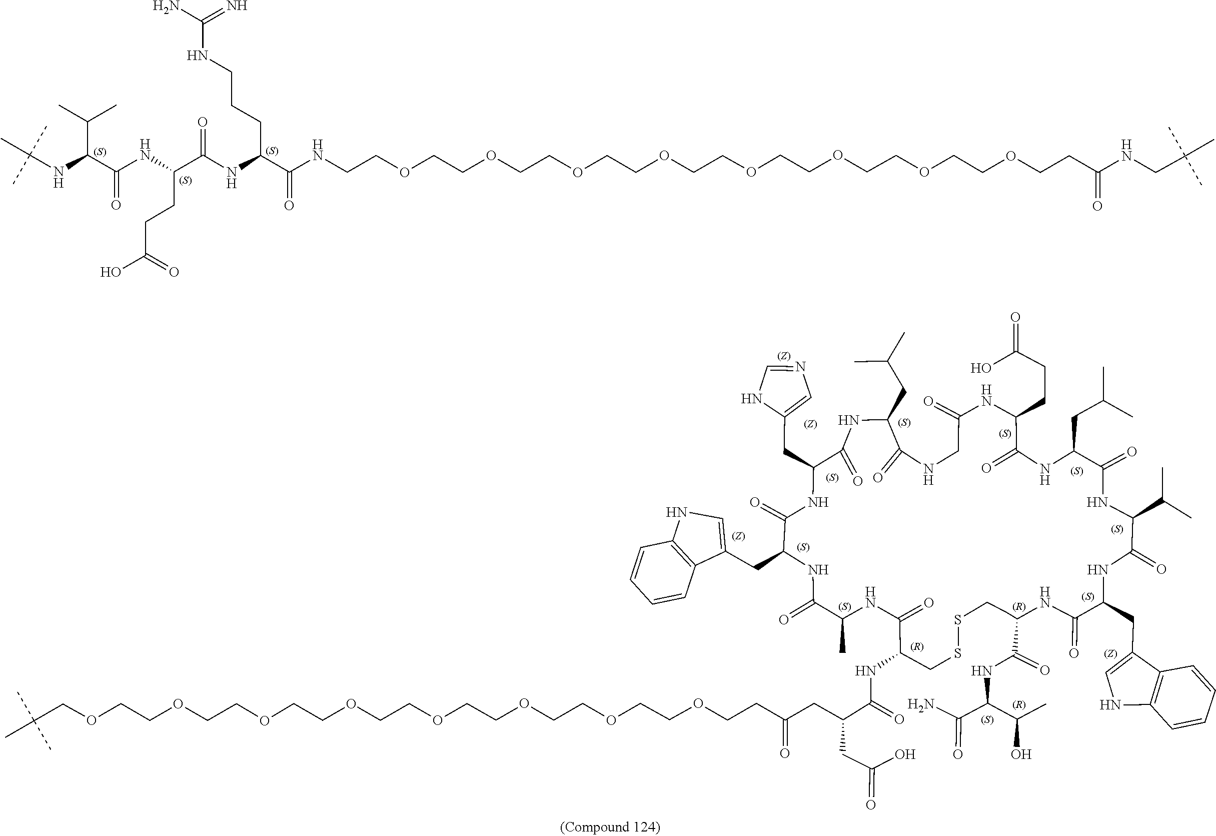

(SEQ ID NO: 9)
Ac-DKEWILQKIYEIMRLLDELGHAEASMRVSDLIYEFMKKGDERLLEEA
ERLLEEVER-[NH-PEG8-C═O]₂-D[CAWHLGELVWC]T-NH2

0.5 mmol resin afforded Compound 125 (Q1: 10.0 mg, 95.3% purity, TFA salt; Q2, 38.3 mg, 94.4% purity, NH₄OAc salt) as a white solid. LCMS: RT=1.12 min, MS cal.: 10095.51, [M/6+H]⁺=1683.47, [M/7+H]⁺=1443.11, [M/8+H]⁺=1262.66.

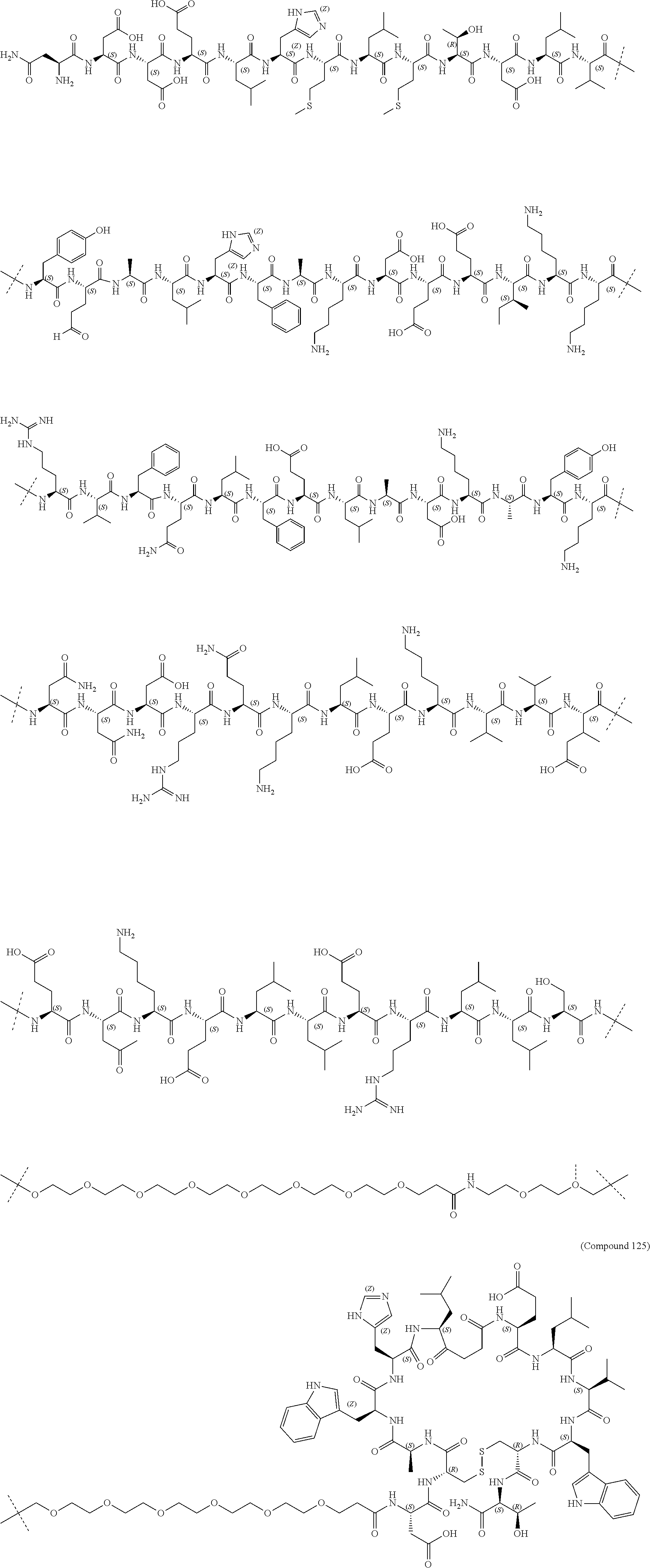

H2N-NDDELHMLMTDLVYEALHFAKDEEIKKRVFQLFELADKAYKNNDR
QKLEKVVEELKELLERLLS-Peg-uABT[(SEQ ID NO:10)-Peg-
uABT]

0.5 mmol resin afforded Compound 126 (Q1: 162.3 mg, 89.5% purity, TFA salt; Q2, 48.0 mg, 93.6% purity, NH₄OAc salt) as a white solid. LCMS: RT=1.12 min, MS cal.: 10137.54, [M/6+H]⁺=1683.47, [M/7+H]⁺=1443.11, [M/8+H]⁺=1262.66.

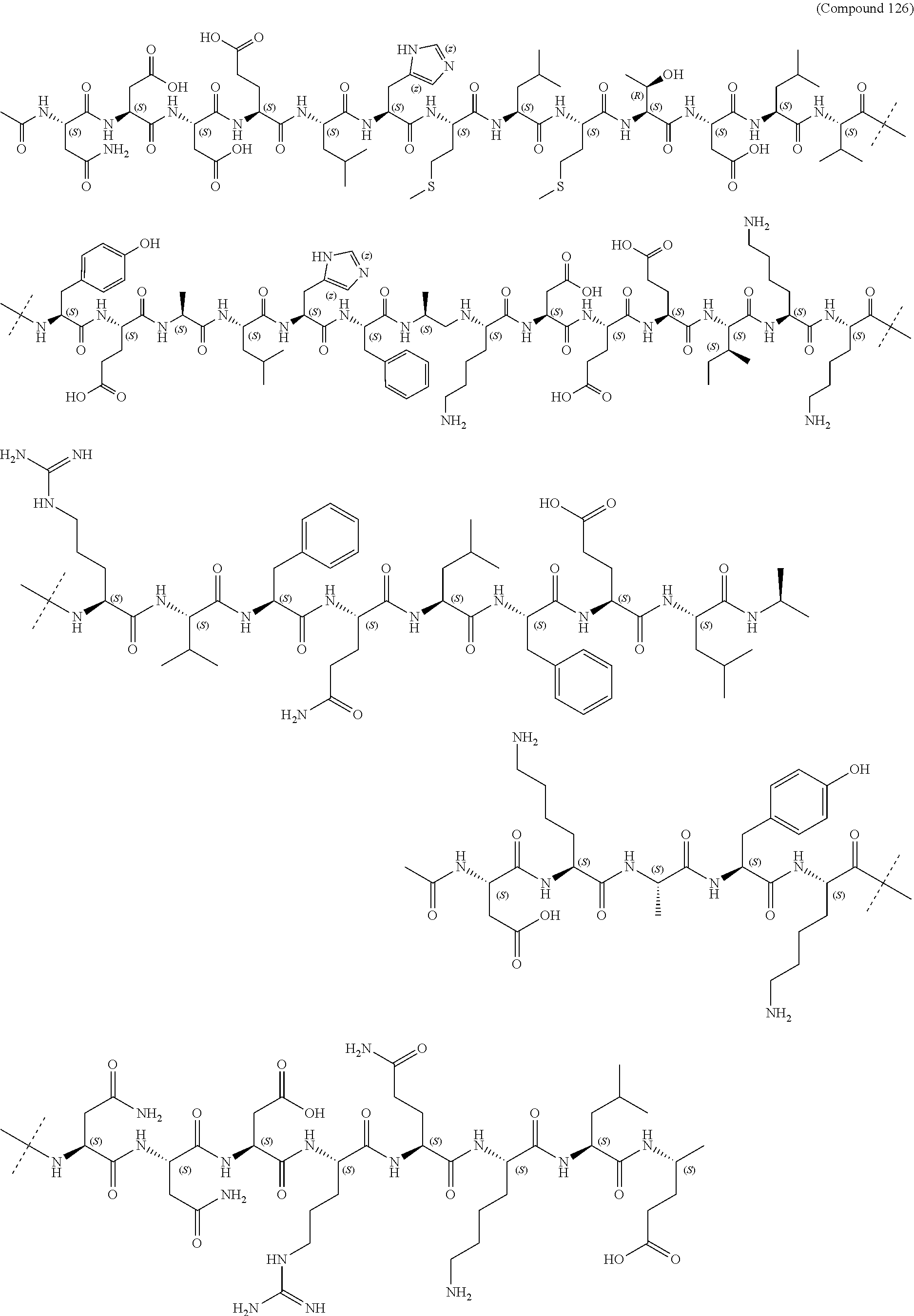

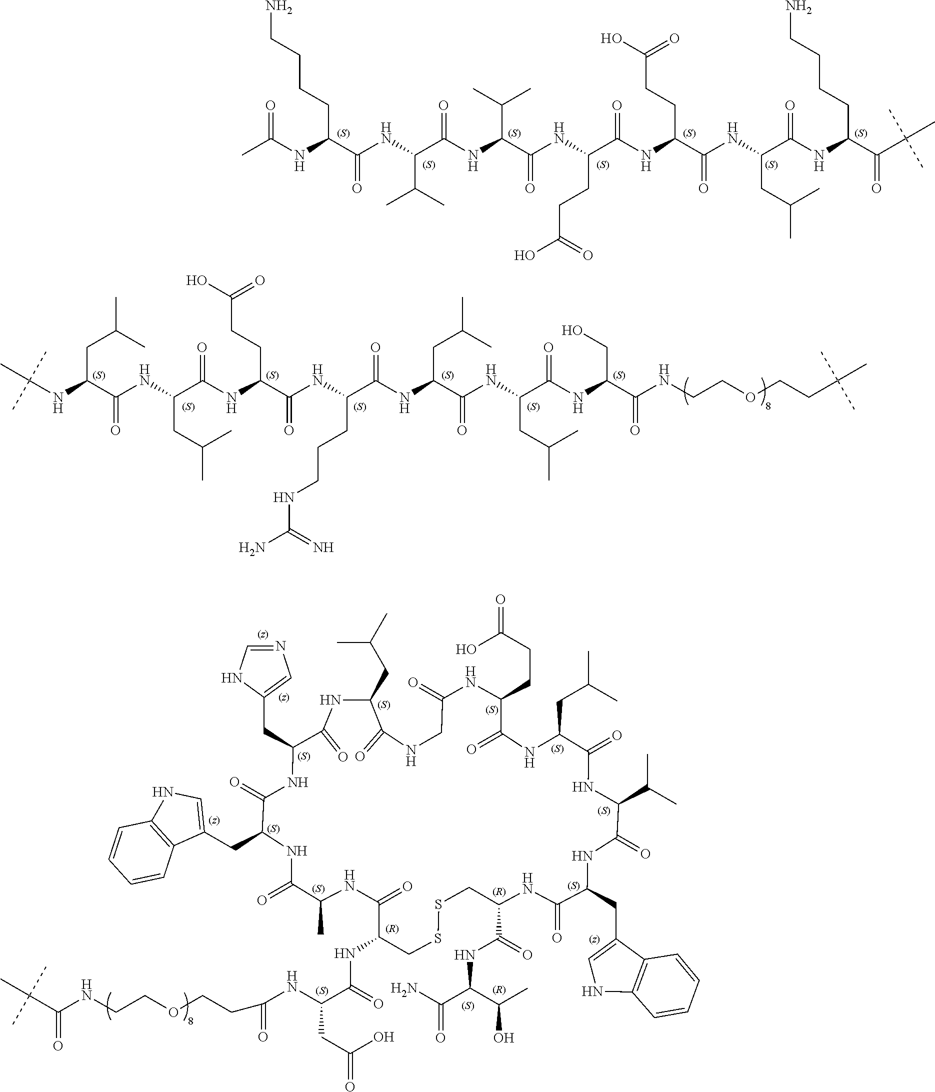

(SEQ ID NO: 11)
Ace-NDDELHMLMTDLVYEALHFAKDEEIKKRVFQLFELADKAYKNNDRQ
KLEKVVEELKELLERLLS-Peg8-Peg8-uABT

### Example 3. Procedure for Preparation of Compound 127 (TFP Fragment Coupling Method to Conjugate with Disulfide Cyclic Peptide)

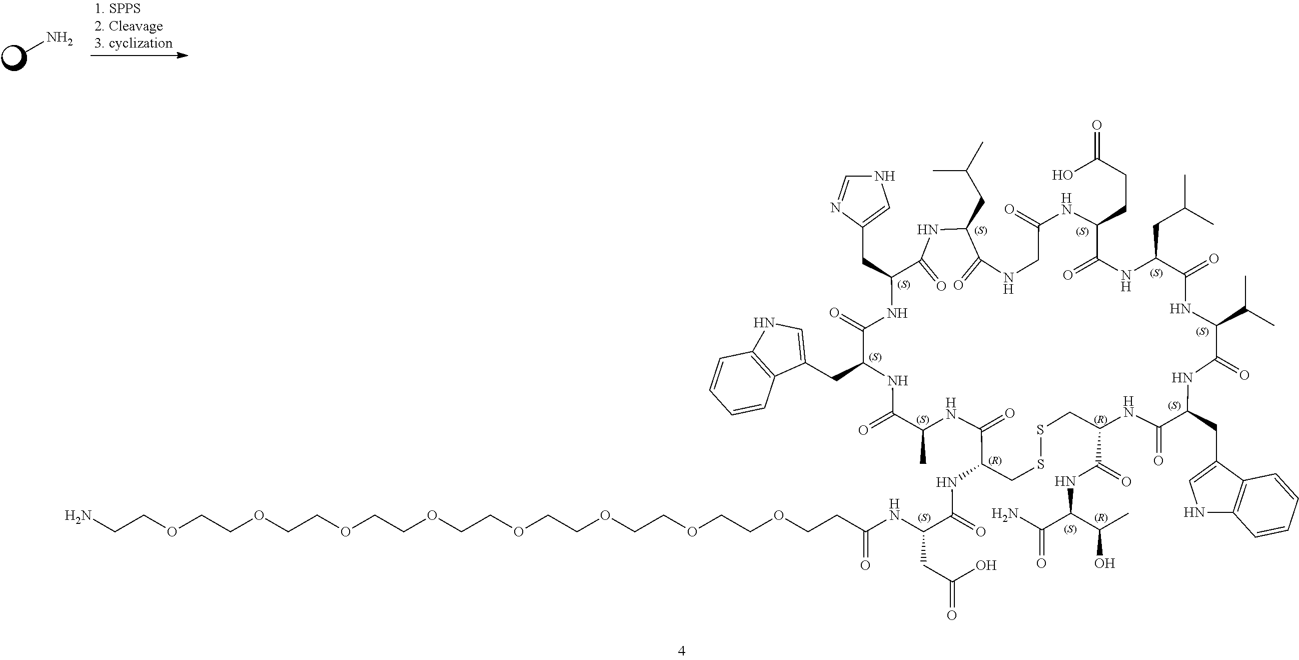

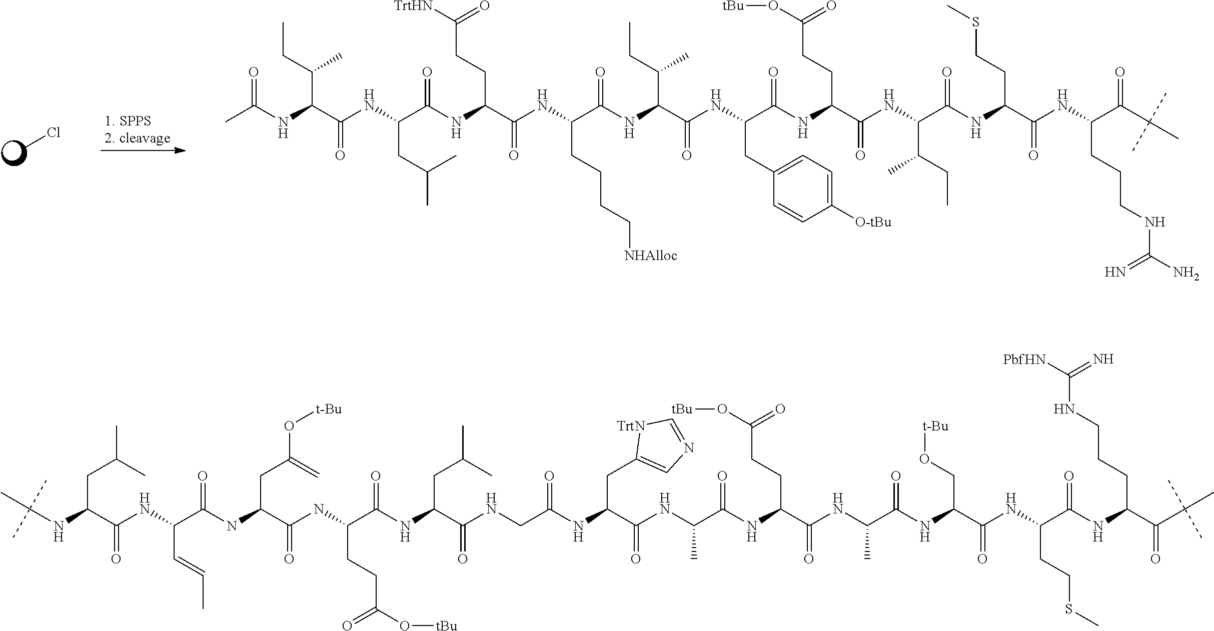

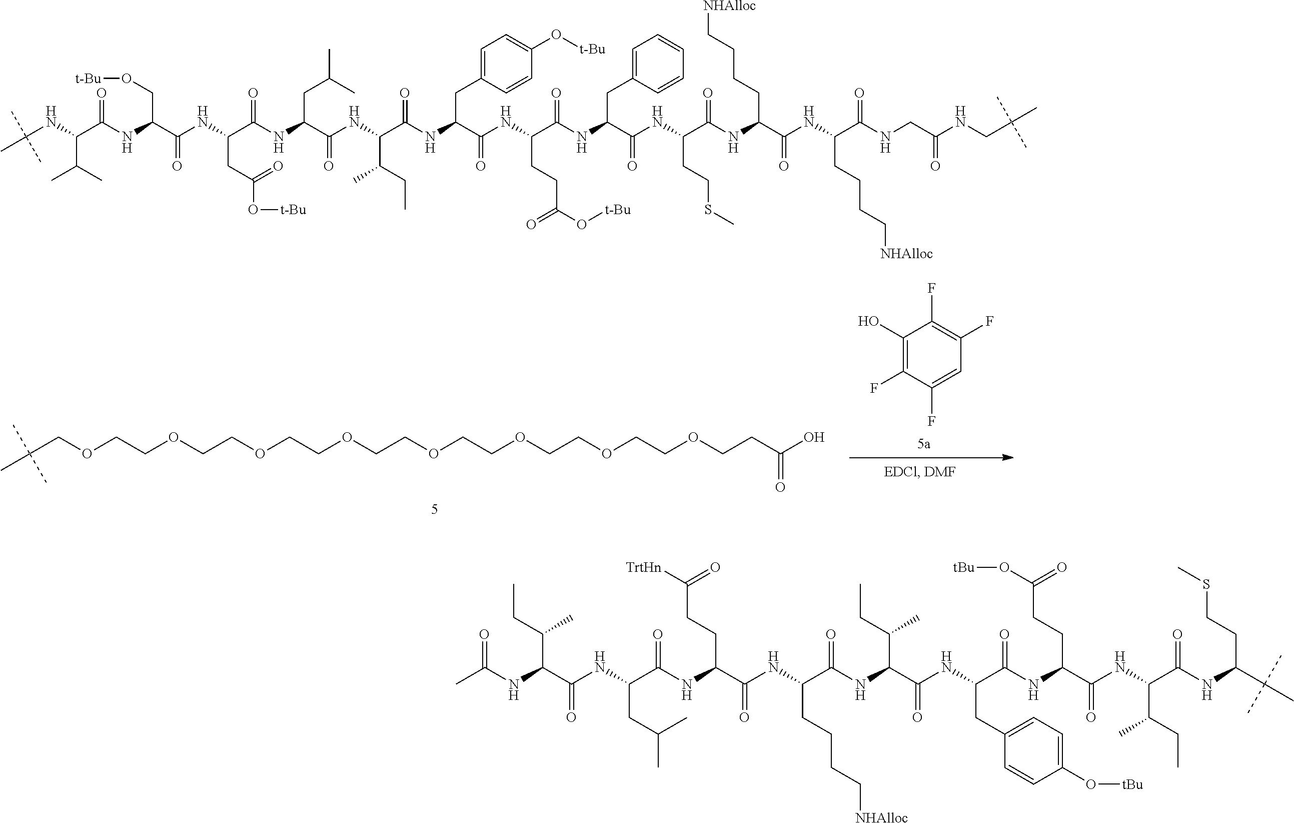

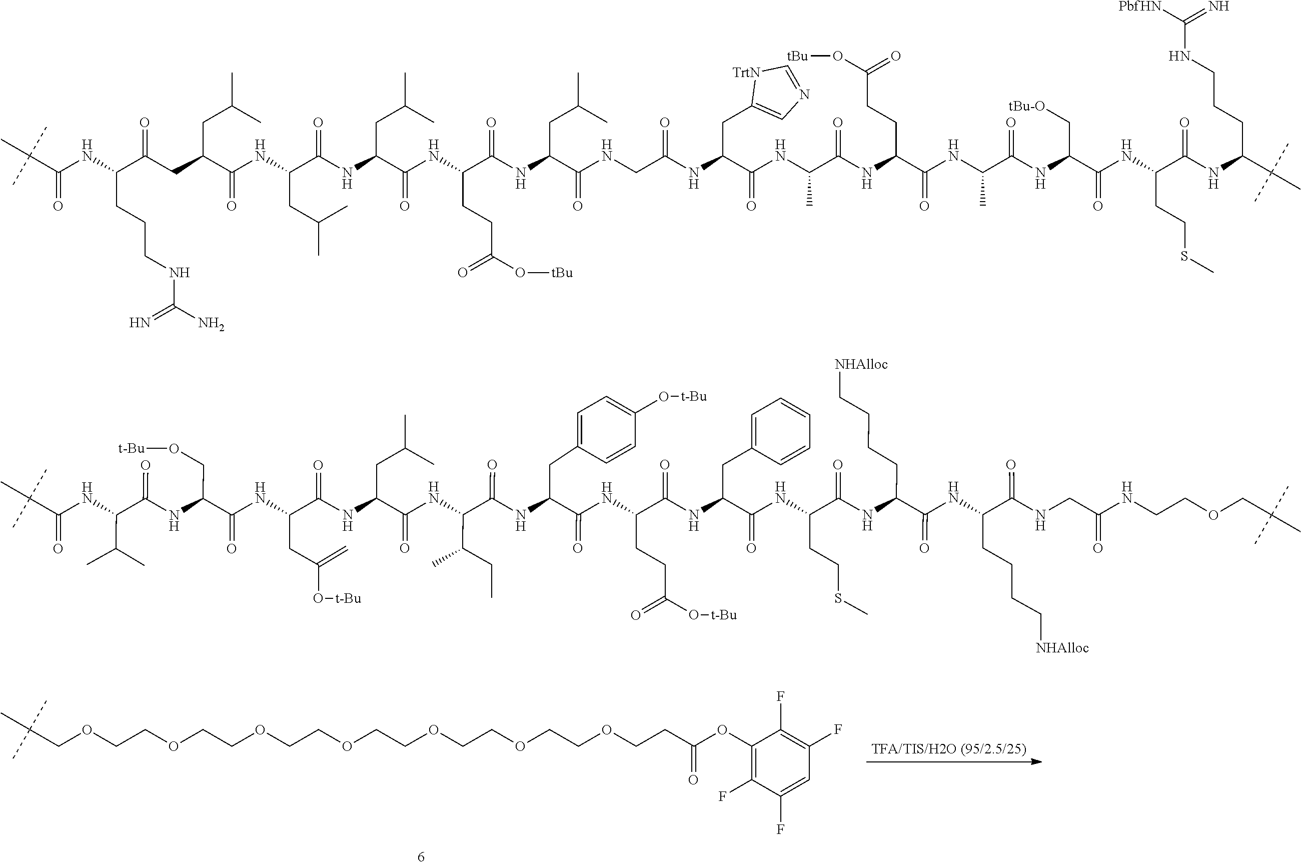

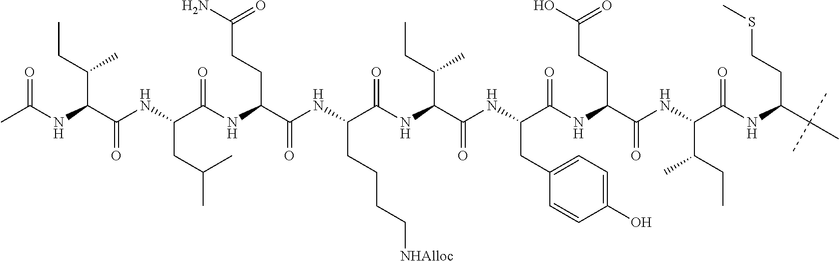

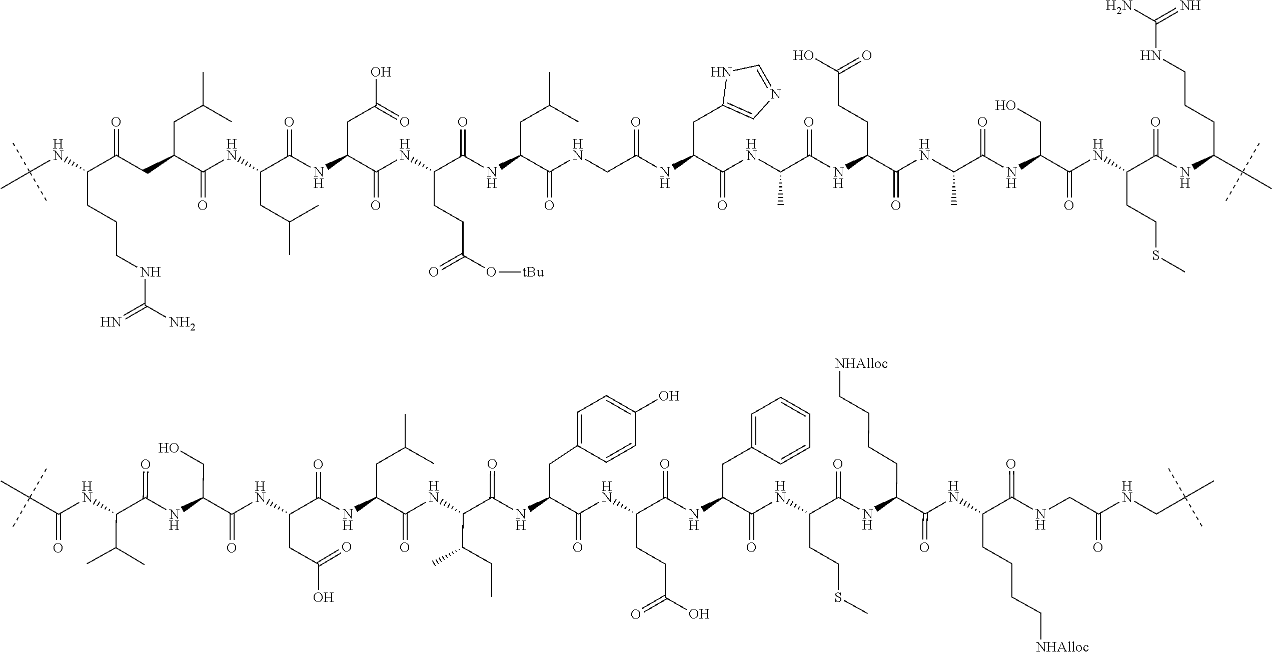

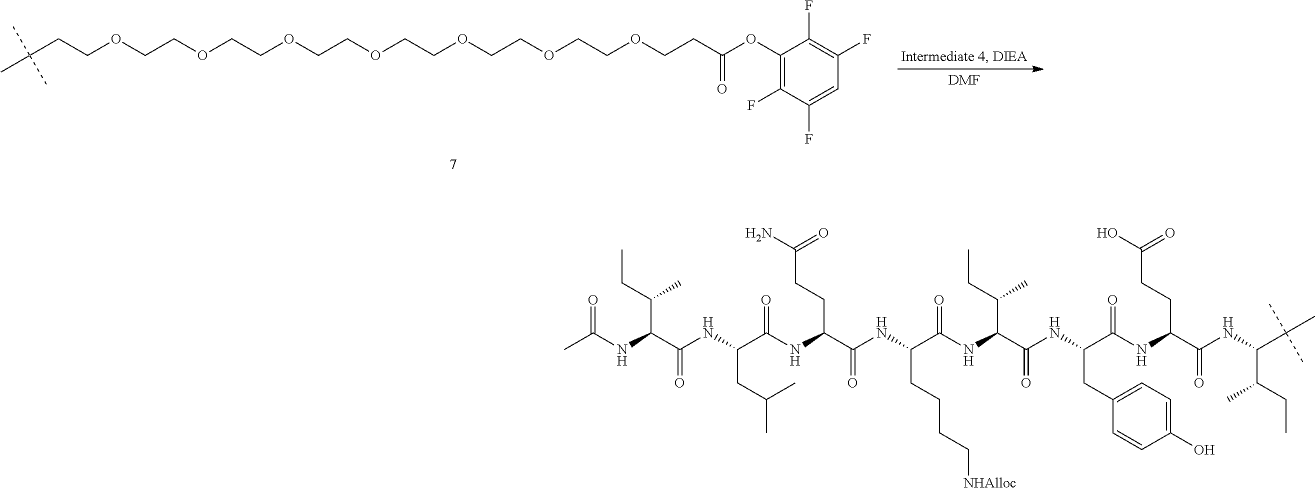

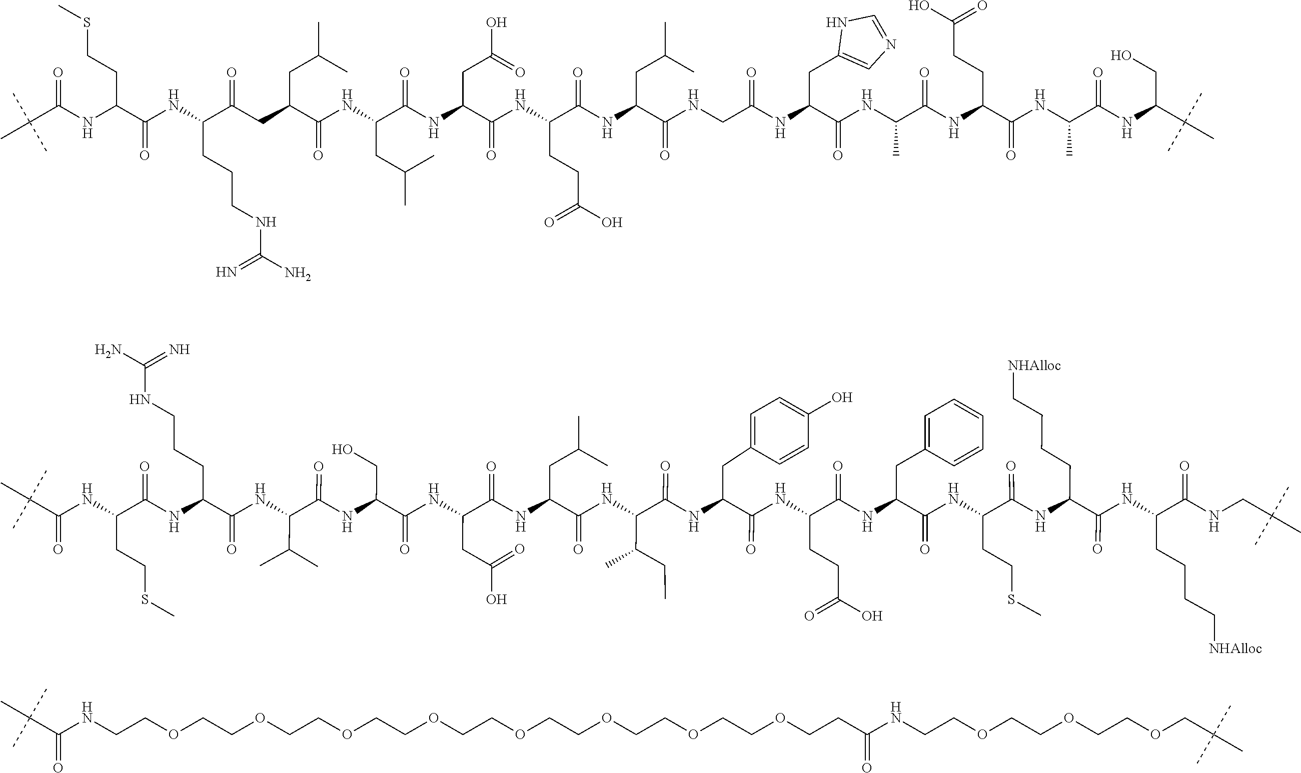

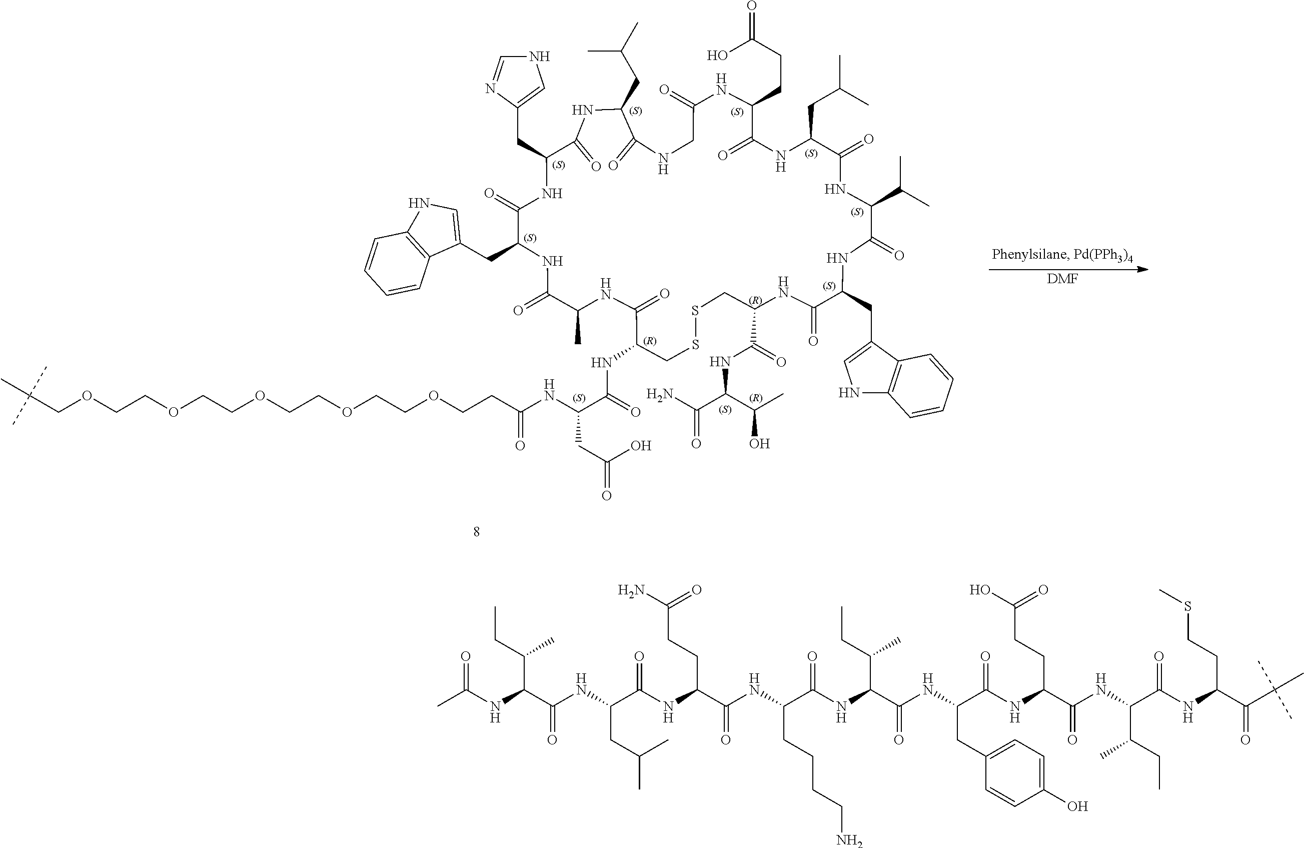

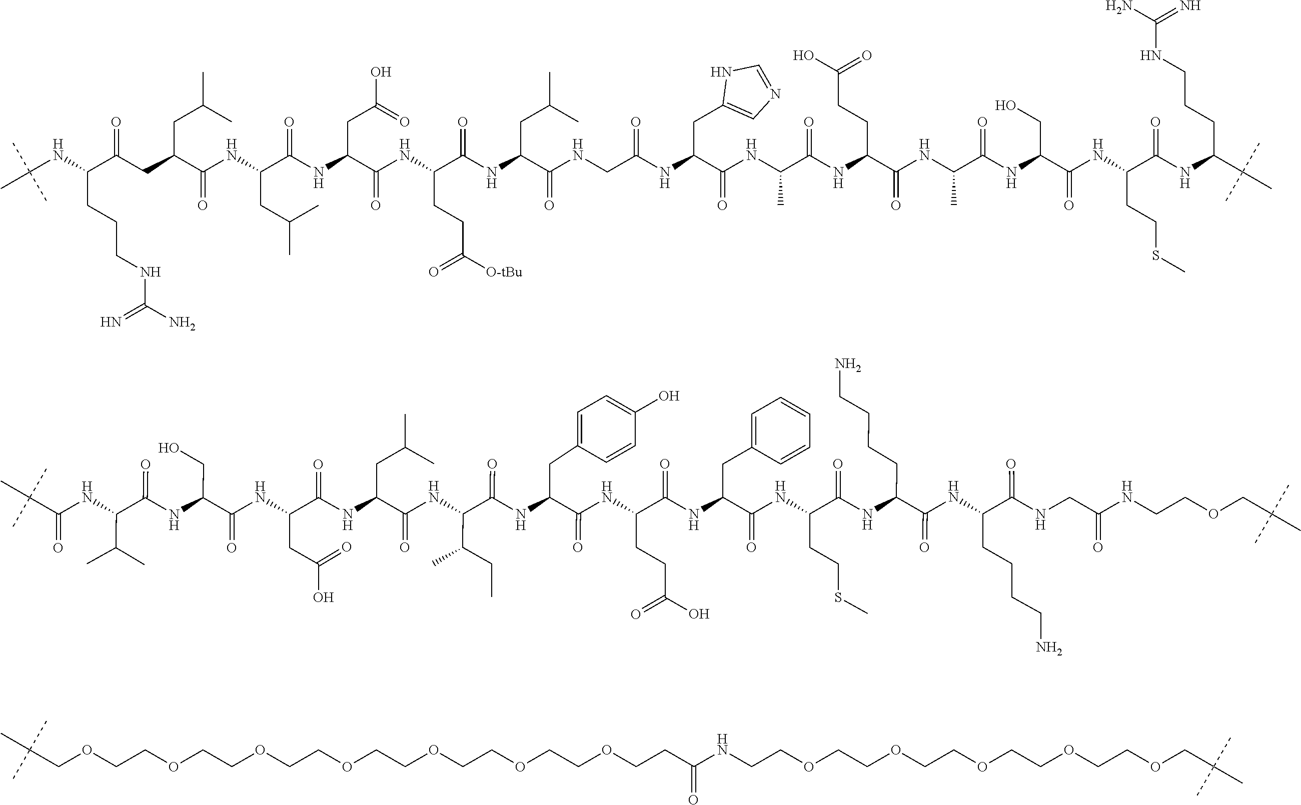

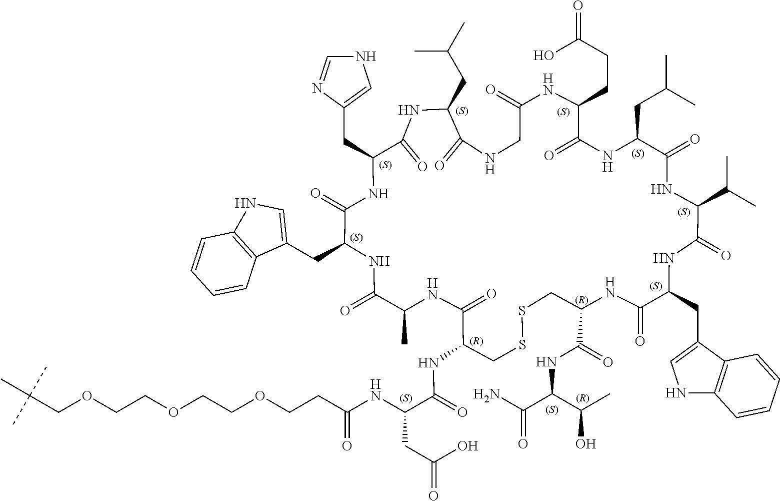

### Preparation of Intermediate 4:

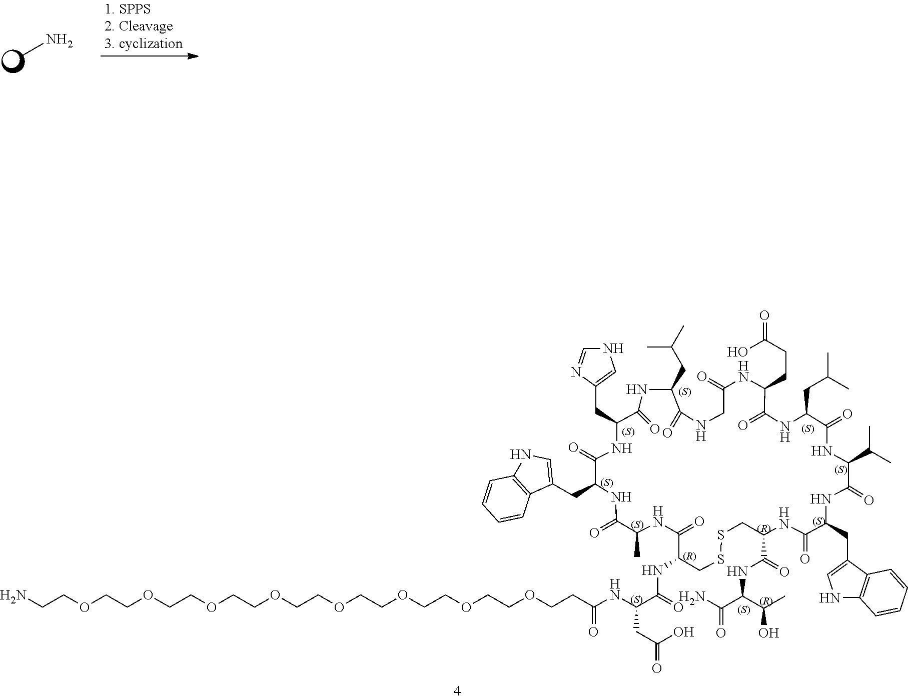

Peptide was synthesized using standard Fmoc chemistry (Rink amide AM resin). Resin preparation: Peptide was added to the vessel containing Rink Amide AM (3.0 mmol, 9.38 g, 0.32 mmol/g) and DMF (30 mL) for 2 hrs with N₂ bubbling at 20° C. Then 20% piperidine in DMF (60 mL) was added and the mixture was bubbled with N₂ for 30 mins at 20° C. The mixture was filtered to obtain the resin. The resin was washed with DMF (60 mL)*5 before proceeding to next step.

Coupling: A solution of Fmoc-Thr(tBu)-OH (3.57 g, 3.00 eq), HBTU (3.25 g, 2.85 eq) in DMF (30 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. The resin was then washed with DMF (60 mL) *5.

Deprotection: 20% piperidine in DMF (60 mL) was added to the resin and the mixture was bubbled with N₂ for 30 mins at 20° C. The deprotection reaction was monitored by ninhydrin test, if it showed blue or other brownish red, the reaction was completed. The resin was then washed with DMF (60 mL)*5.

The coupling and deprotection steps were repeated for the following amino acids: number #3-13, in Table 2.

Coupling: A solution of Fmoc-PEGs-CH₂CH₂COOH (2.98 g, 1.50 eq), HATU (1.64 g, 1.42 eq) in DMF (30 mL) was added to the resin with N₂ bubbling. Then DIEA (1.16 g, 3.00 eq) was added to the mixture dropwise and bubbled with N₂ for 30 mins at 15° C. The coupling reaction was monitored by ninhydrin test, if it showed colorless, the coupling was completed. The resin was then washed with DMF (60 mL)*5.

Repeat the deprotection step for Fmoc removal.

After the last position is completed, the resin was then washed with DMF (60 mL)*5, MeOH (60 mL)*5, and then dried under vacuum to afford peptide resin (AM resin, 3 mmol, 15.5 g).

TABLE 2

| # | Materials | Coupling reagents |
|---|---|---|
| 1 | Fmoc-Thr(OtBu)-OH (3.00 eq) | HBTU (2.85 eq) and DIEA (6.00 eq) |
| 2 | Fmoc-Cys(Trt)-OH (3.00 eq) | HBTU (2.85 eq) and DIEA (6.00 eq) |
| 3 | Fmoc-Trp-OH (3.00 eq) | HBTU (2.85 eq) and DIEA (6.00 eq) |
| 4 | Fmoc-val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | Fmoc-His(Trt)-OH (3.00 eq) | HBTU (2.85 eq) and DIEA (6.00 eq) |
| 10 | Fmoc-Trp-OH (3.00 eq) | HBTU (2.85 eq) and DIEA (6.00 eq) |
| 11 | Fmoc-Ala-OH (3.00 eq) | HBTU (2.85 eq) and DIEA (6.00 eq) |
| 12 | Fmoc-Cys(Trt)-OH (3.00 eq) | HBTU (2.85 eq) and DIEA (6.00 eq) |
| 13 | Fmoc-Asp(OtBu)-OH (3.00 eq) | HBTU (2.85 eq) and DIEA (6.00 eq) |
| 14 | Fmoc-PEG8-CH2CH2COOH | HATU (1.42 eq) and DIEA (3.00 eq) |
| | (1.50 eq) | |

Peptide Cleavage, cyclization and purification Cleavage buffer (95% TFA/2.5% TIS/2.5% H₂O, 150 mL) was added to the flask containing the side chain protected peptide resin at room temperature (20° C.) and stirred for 2 hrs. After filtration, the filtrate was collected. The peptide was precipitated with cold isopropyl ether (1000 mL).

After filtration, the solid was dried under vacuum for 2 hrs to afford crude peptide (6.0 g, crude) as a white solid.

The crude peptide was dissolved in MeCN/H₂O (3:7, 3000 mL), then the mixture was added 0.1 M I₂/HOAc dropwise until the yellow color persisted, then the mixture was stirred at 20° C. for 5 mins. The mixture was quenched with 0.1 M Na₂S₂O₃ dropwise until yellow color disappeared. After filtration, the filtrate was purified by prep-HPLC (acidic conditions, TFA) directly, followed by lyophilization to afford Intermediate 4 (1.20 g, 93.2% purity, 19.1% yield) as a white solid. LCMS: RT=1.52 min, MS cal.: 1953.24, [M/2+H]⁺=977.17, [M/3+H]⁺=651.80, [M/6+H]⁺=1133.36.

### Preparation of Intermediate 5:

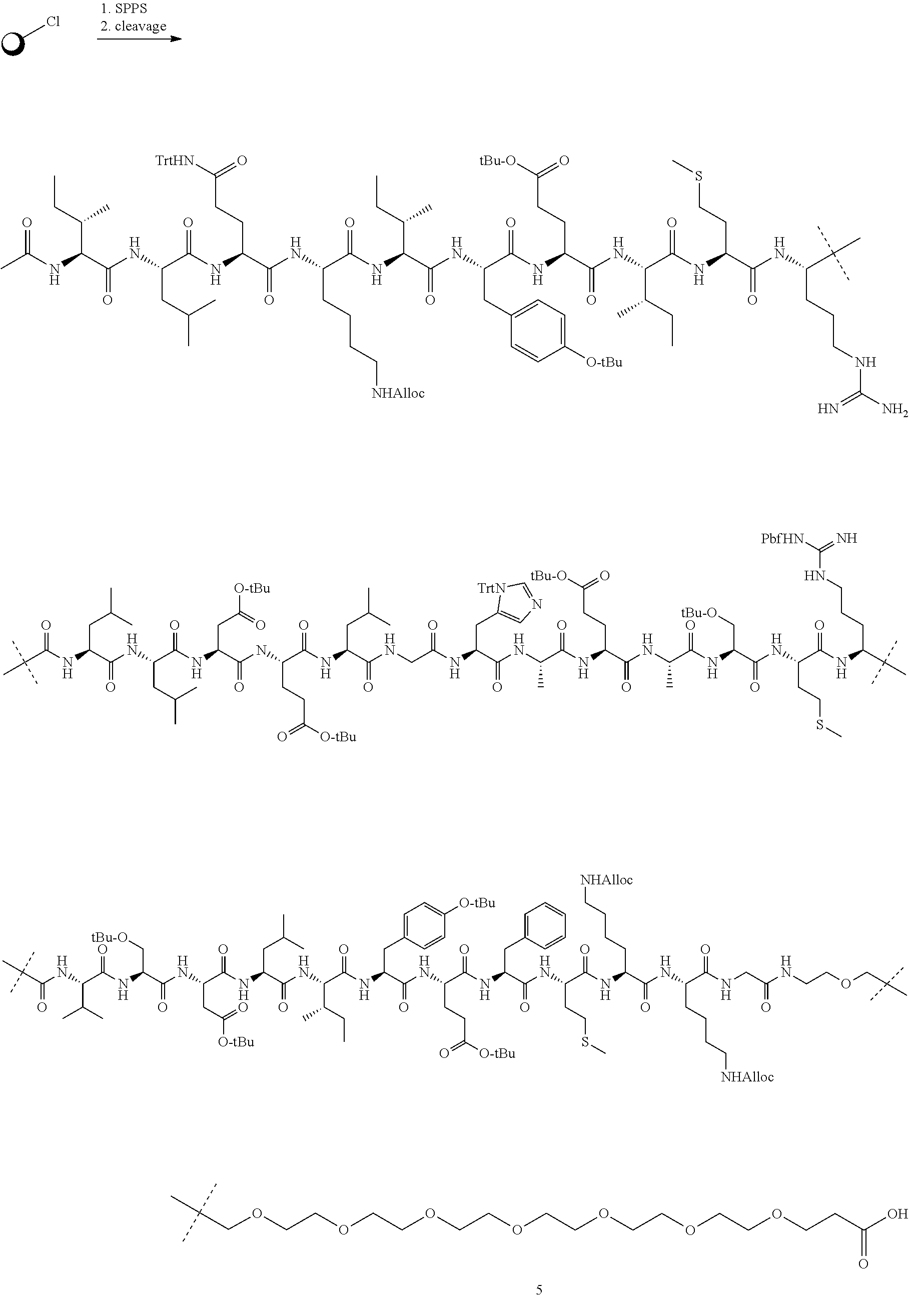

The fully protected resin was synthesized using standard Fmoc chemistry (e.g. see Example 1).

Peptide Cleavage and Purification: Cleavage buffer (20% HFIP/DCM, 20 mL) was added to the flask containing the side chain protected peptide resin (0.5 mmol, 3.5 g) at room temperature and stirred for 1 hr. After filtration, the filtrate was collected. Repeat the cleavage step twice. The combined filtrates were concentrated under reduced pressure and the residue was dried under lyophilization to afford Intermediate 5 (2.5 g, crude) as a white solid.

### Preparation of Intermediate 6:

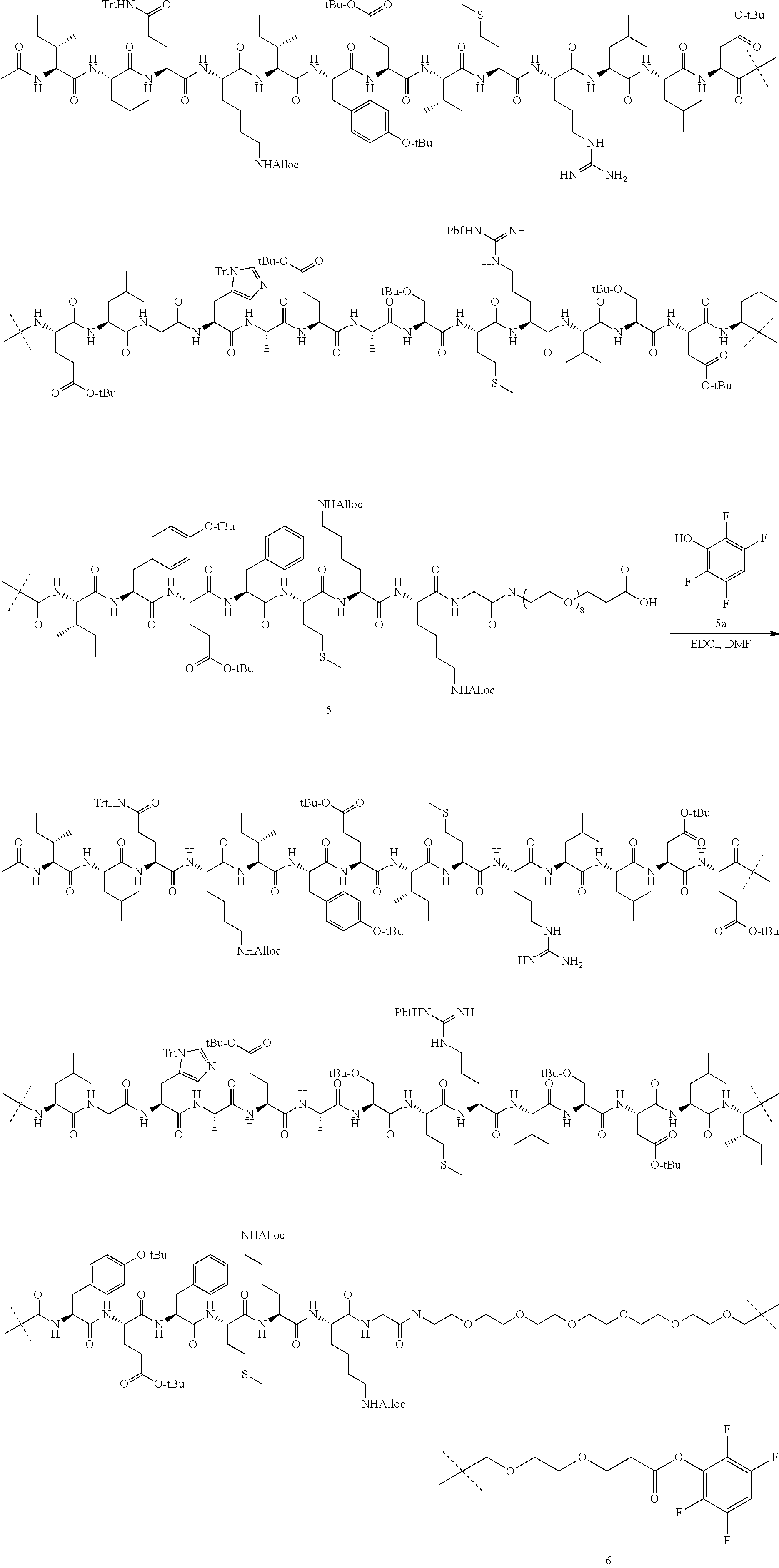

To a mixture of Intermediate 5 (2.5 g, crude), Intermediate 5a (401.9 mg, 6.00 eq) in DMF (20 mL) was added EDCI (232.6 mg, 3.00 eq) at 20° C. The mixture was stirred at 20° C. for 3 hrs. The mixture was precipitated with 0.1 M HCl (cold, 200 mL). After filtration, the solid was dried by lyophilization to afford Intermediate 6 (2.5 g, crude) as a white solid.

### Preparation of Intermediate 7:

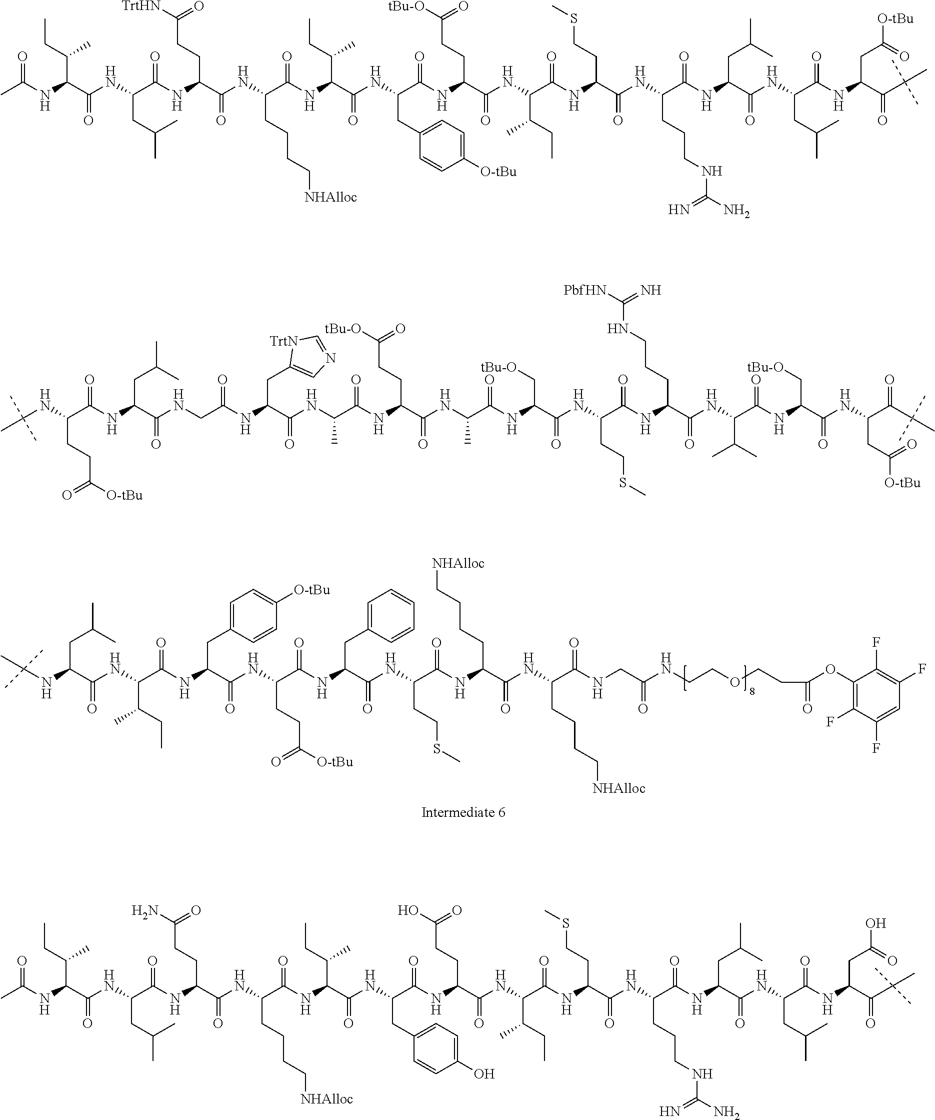

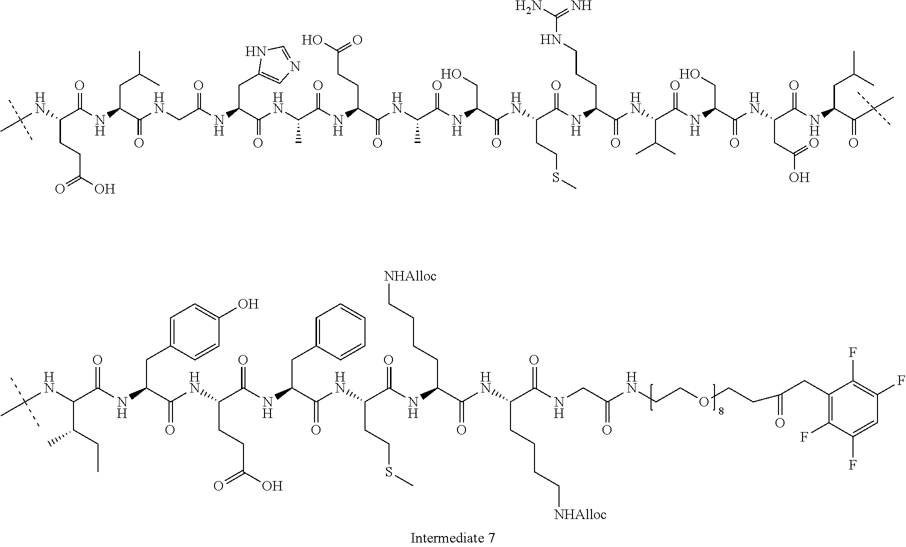

A mixture of Intermediate 6 (2.5 g, crude) in TFA/TIS/H₂O (95/2.5/2.5, 50 mL) was stirred at 20° C. for 1 h. The mixture was precipitated with cold isopropyl ether (100 mL) and centrifuged (3 mins at 3000 rpm). Washed with Isopropyl ether two additional times, and the crude peptide was dried under vacuum for 2 hrs. The crude peptide was purified by prep-HPLC (acidic conditions, TFA), followed by lyophilization to afford Intermediate 7 (900 mg, 91.5% purity) as a white solid. LCMS: RT=1.35 min, MS cal.: 5007.69, [M/3+H]⁺=1670.14, [M/4+H]⁺=1252.77, [M/5+H]⁺=1002.3.

### Preparation of Intermediate 8:

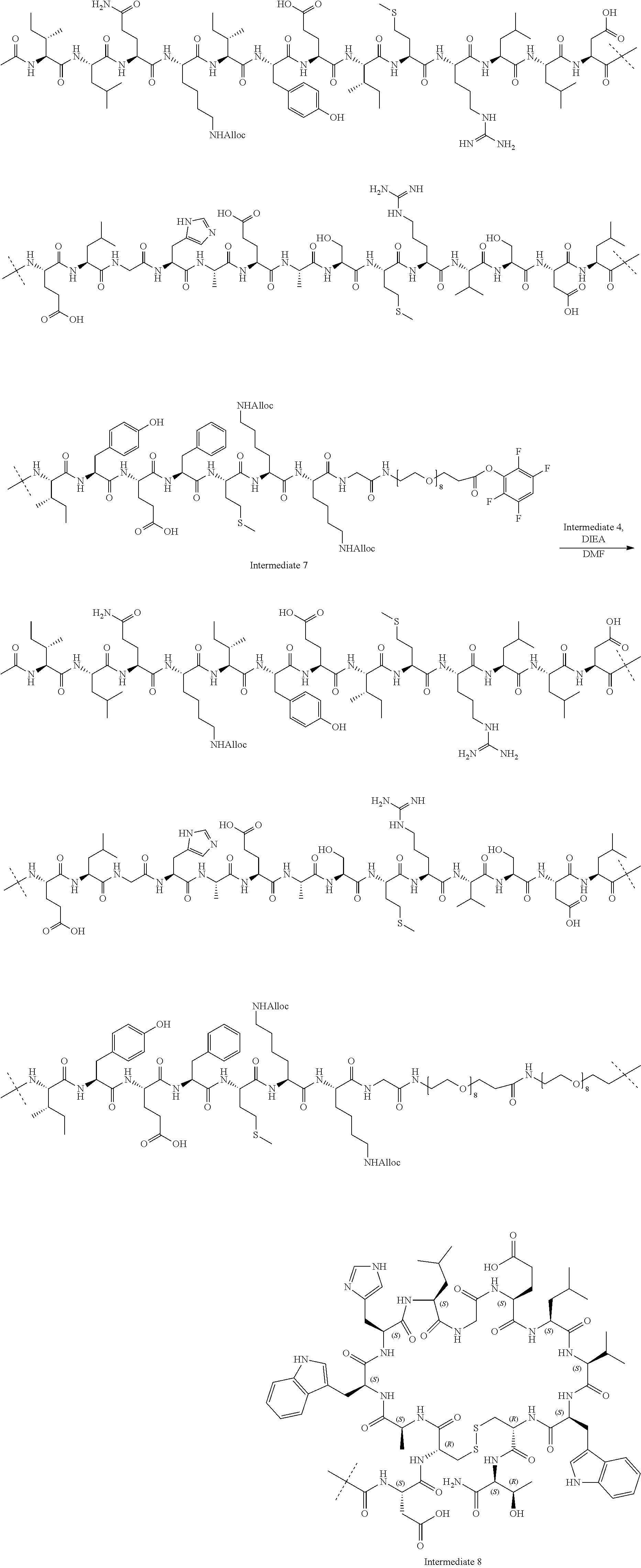

To a solution of Intermediate 7 (153.7 mg, 30.7 μmol, 1.20 eq), Intermediate 4 (50.0 mg, 25.5 μmol, 1.00 eq) in DMF (1 mL) was added DIEA (19.84 mg, 153.51 μmol, 26.74 uL, 6.00 eq) at 20° C. The mixture was stirred at 20° C. for 1 hr. The mixture was then purified by prep-HPLC (TFA condition) to afford Intermediate 8 (76.0 mg, 88.8% purity, 38.8% yield) as a white solid. TOF: RT=1.52 min, MS cal.: 6794.85, [M/4+H]⁺=1699.80, [M/5+H]⁺=1359.64, [M/6+H]⁺=1133.36.

### Preparation of Compound 127:

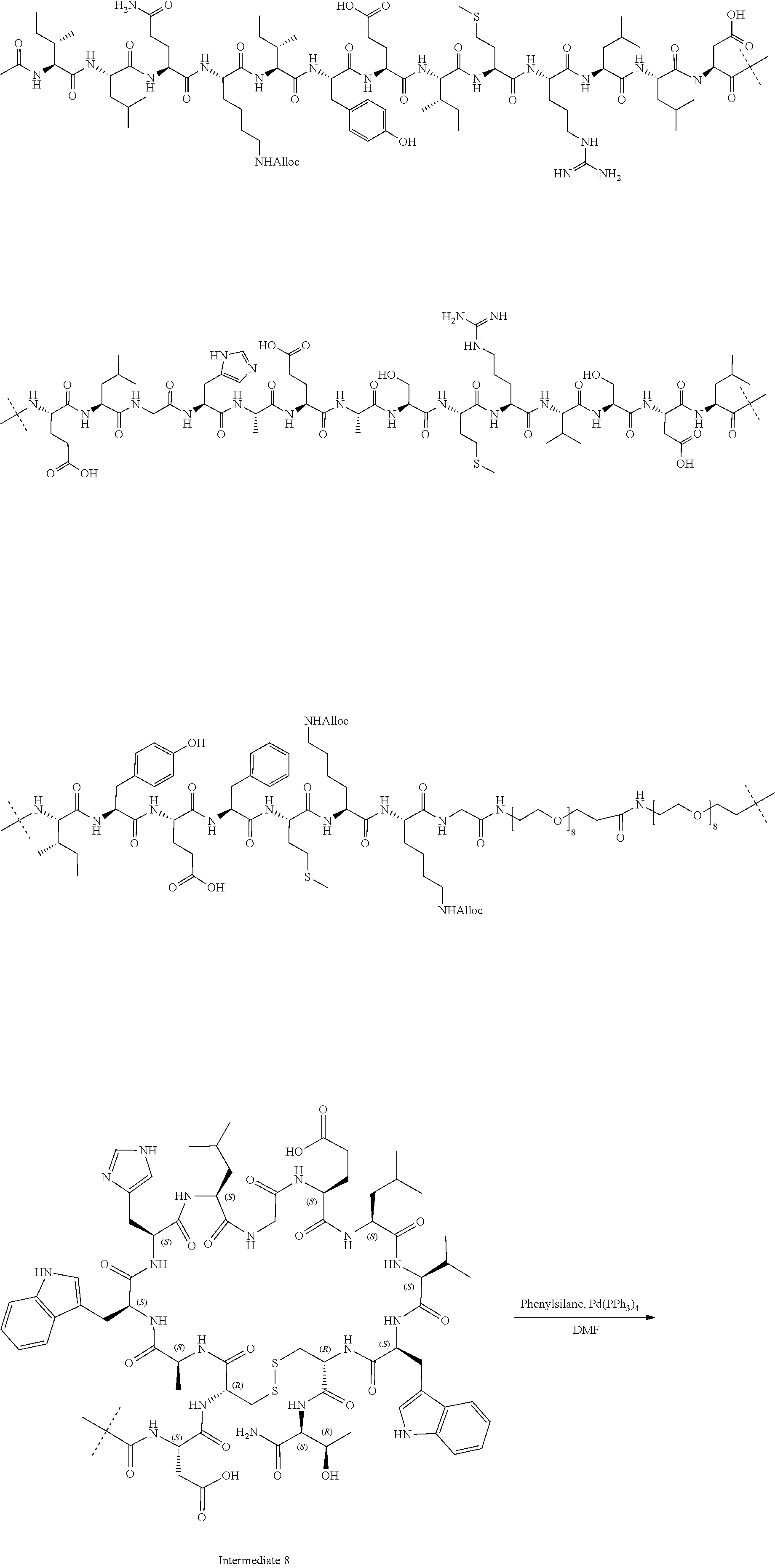

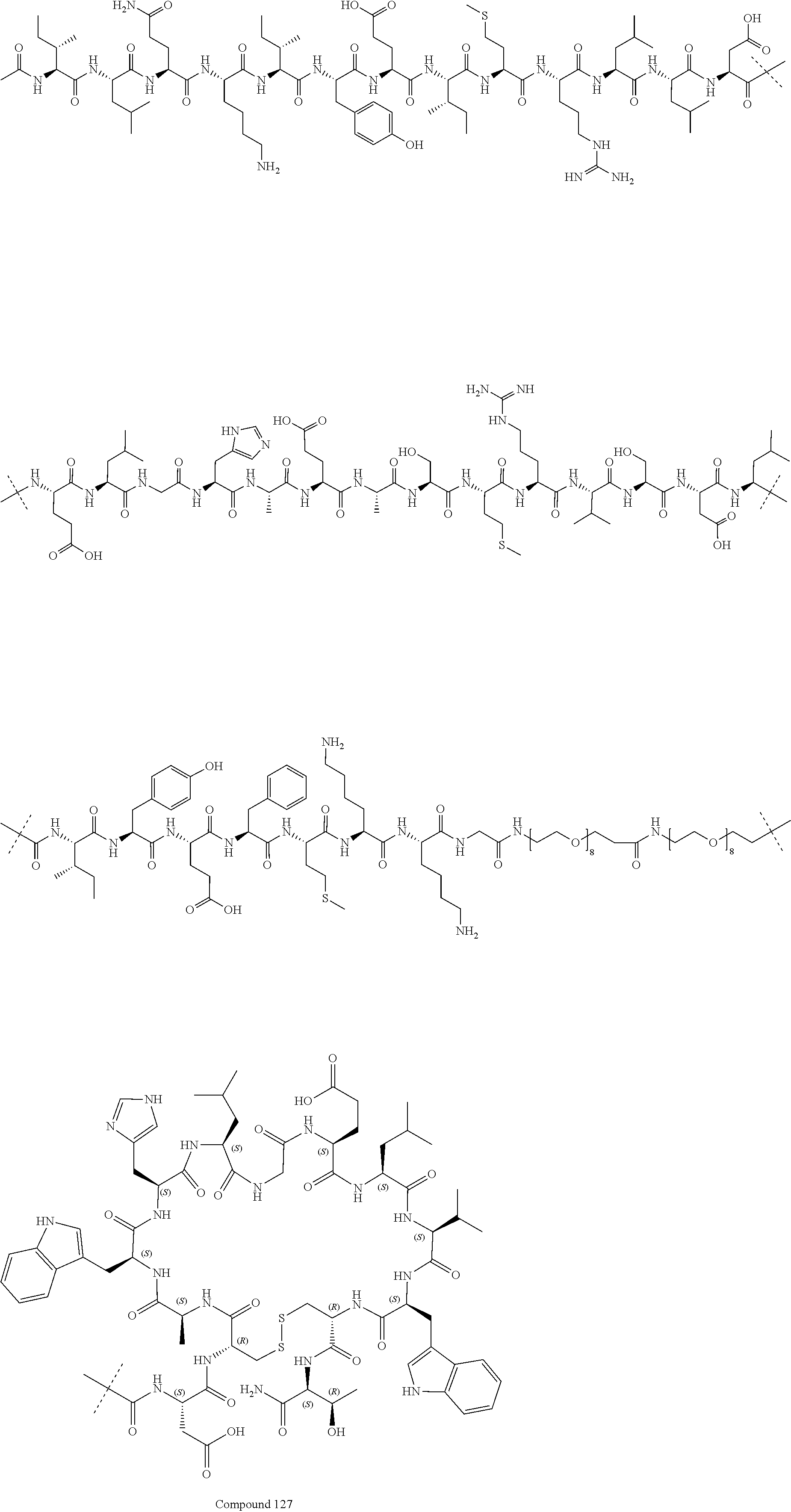

To a solution of Intermediate 8 (76.0 mg, 11.18 μmol, 1.00 eq), phenylsilane (12.1 mg, 111.8 μmol, 13.8 μL, 10.00 eq), Pd(PPh₃)₄ (6.4 mg, 5.5 μmol 1, 0.50 eq) in DMF (1 mL) was stirred at 20° C. under N₂ atmosphere for 1 hr. The mixture was purified by prep-HPLC (TFA condition) directly to afford Compound 127 (31.8 mg, 4.75 μmol, 97.7% purity, 42.4% yield) as a white solid. LCMS: RT=2.26 min, MS cal.: 6542.63, [M/4+H]⁺=1636.3, [M/5+H]⁺=1309.2, [M/6+H]⁺=1091.2.

### Example 4. Procedure for Preparation of Compound 128 (Bi-TFP Fragment Coupling Method to Conjugate Linear Fragment with Disulfide Cyclic Peptide)

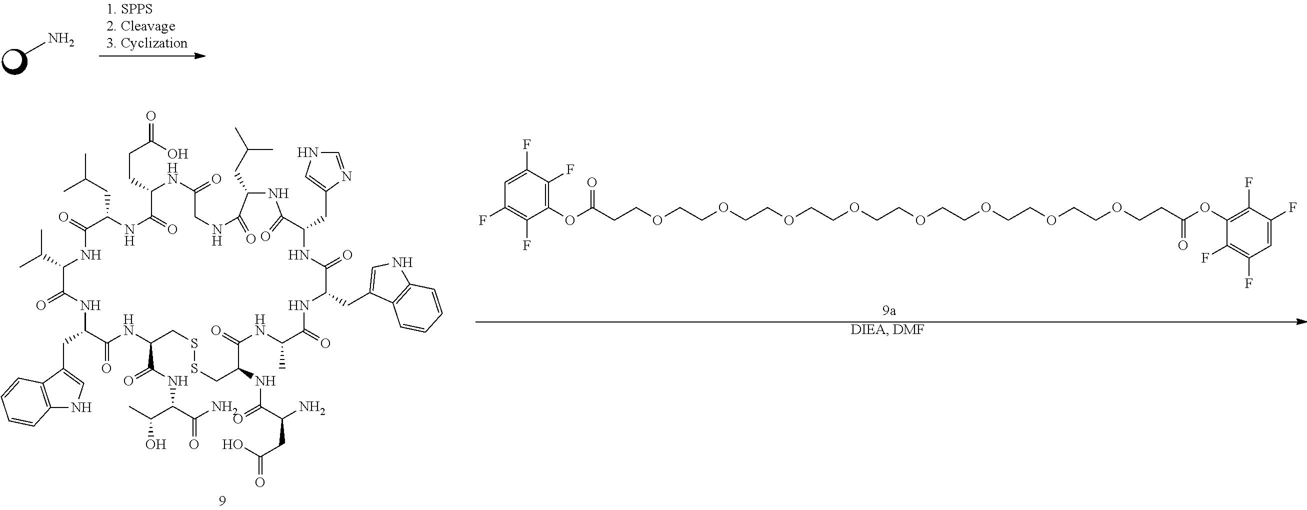

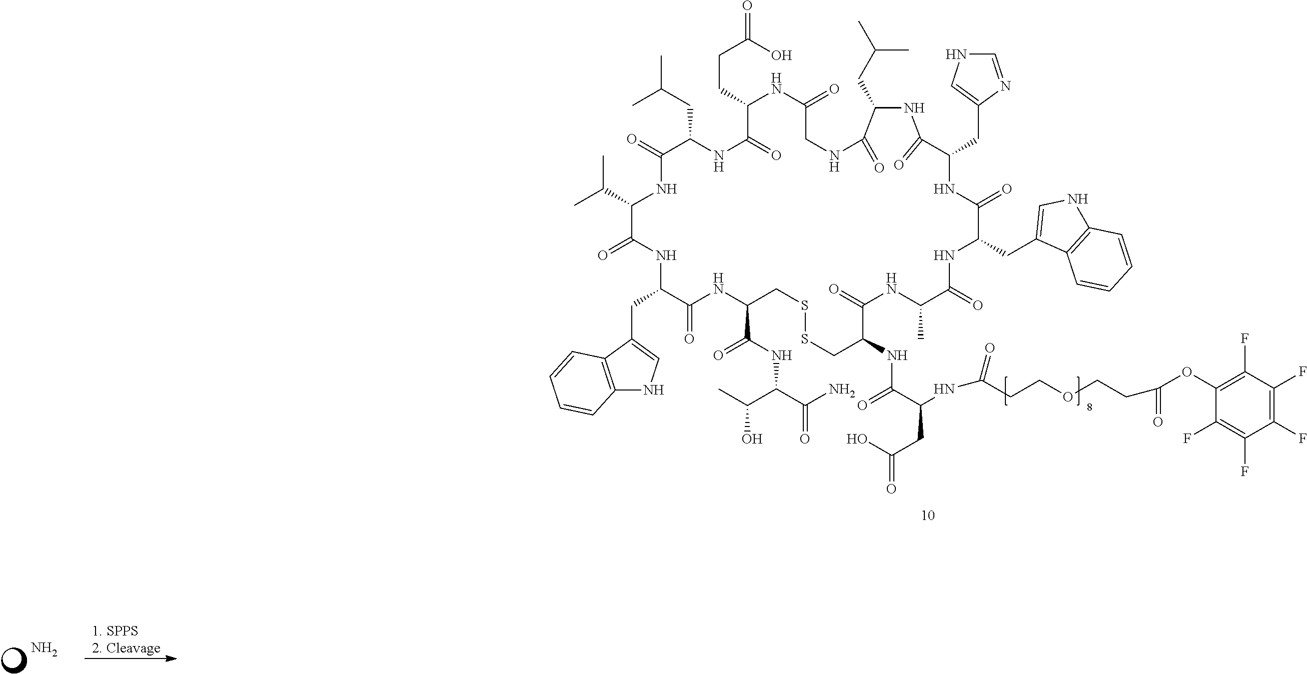

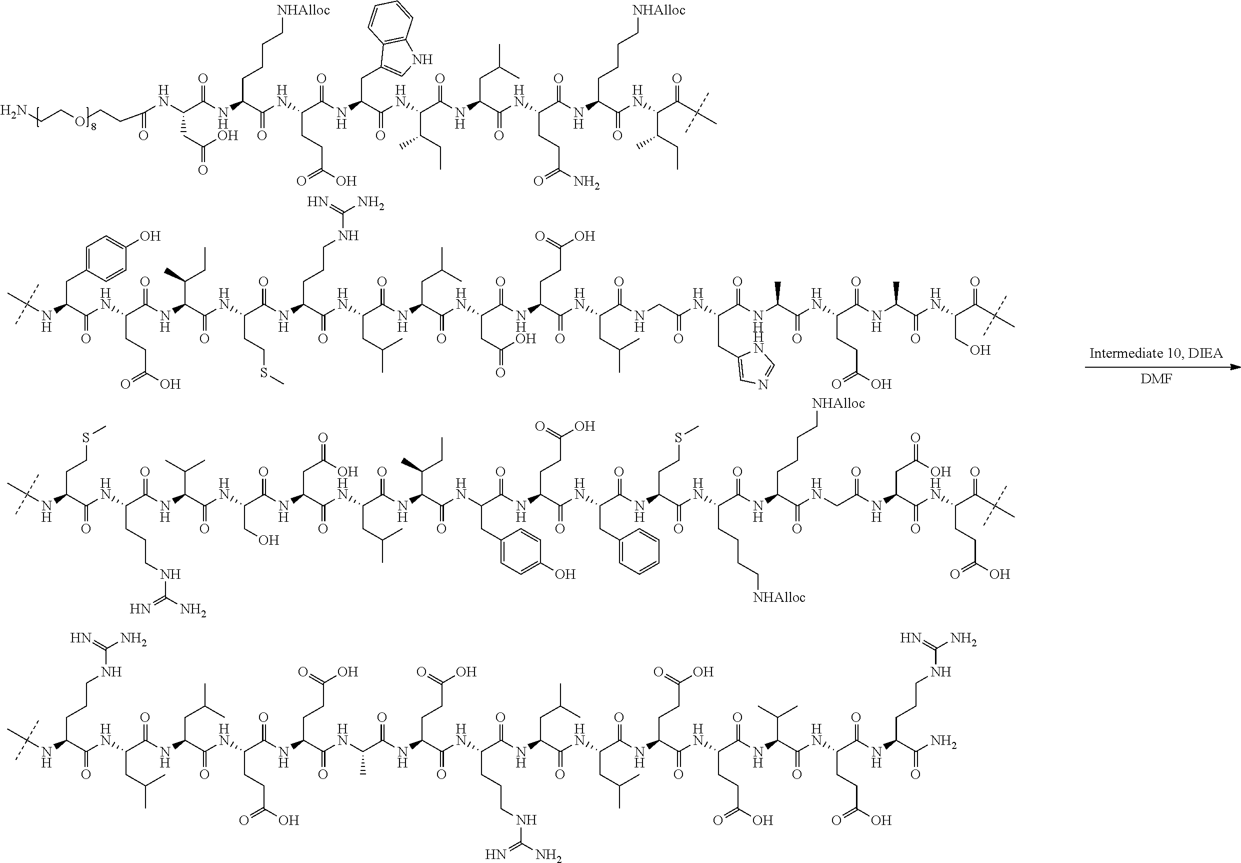

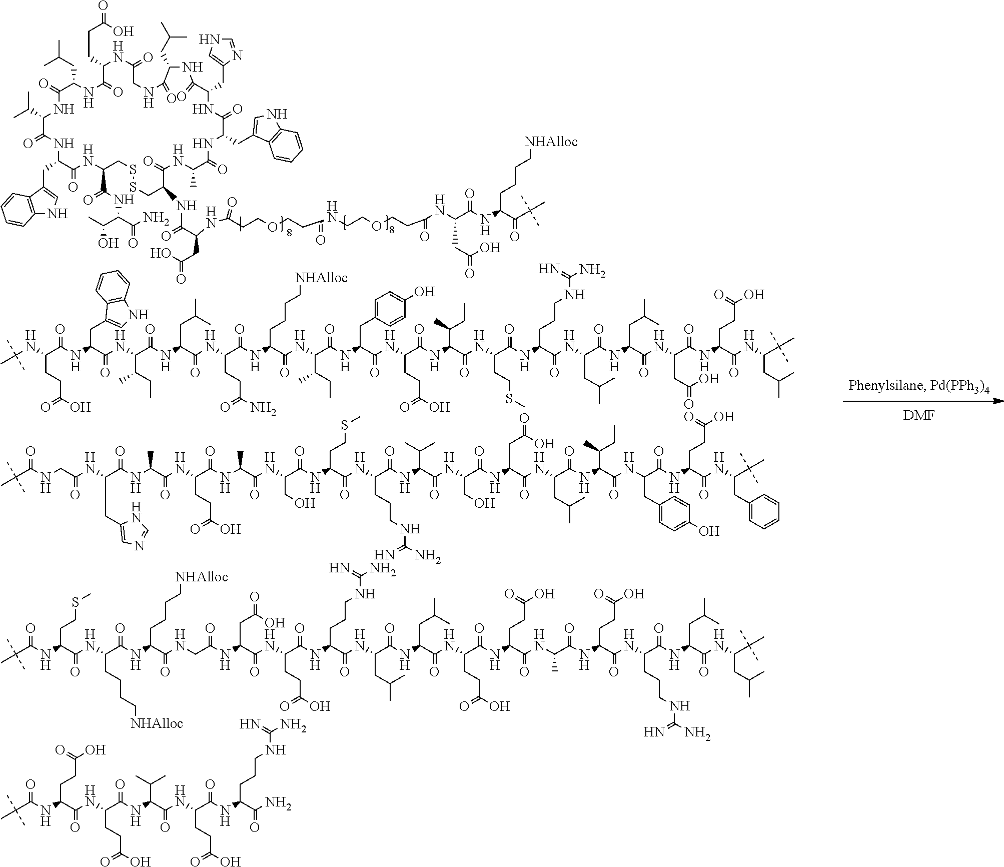

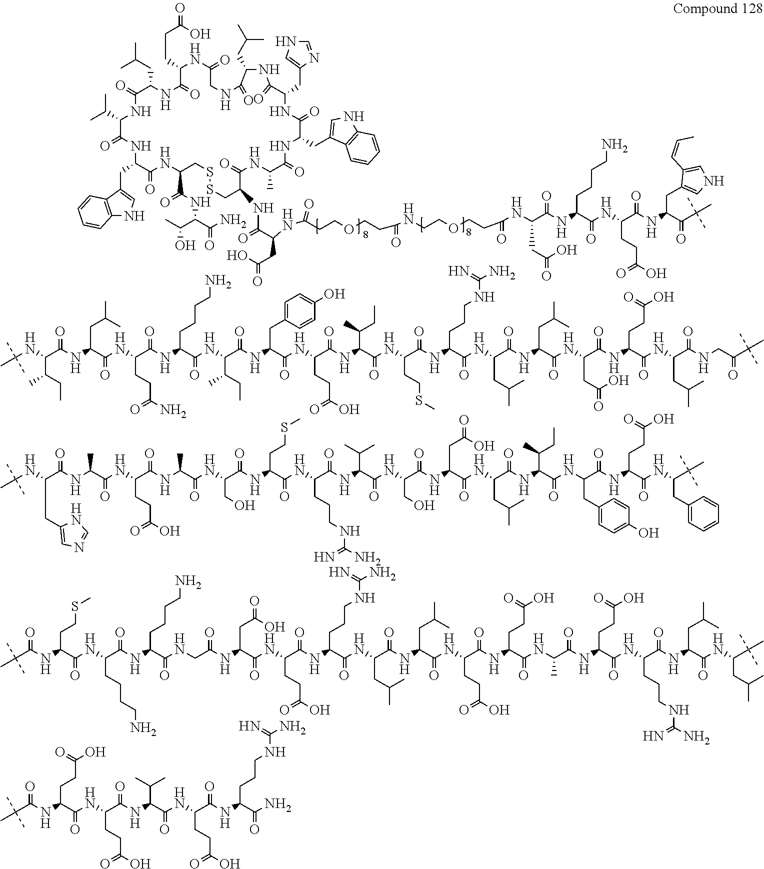

### Preparation of Intermediate 9:

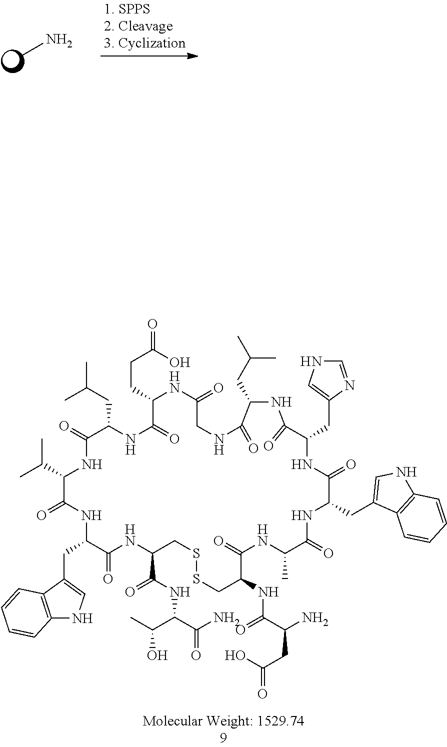

Intermediate 9 was synthesized using the same procedure (SPPS, Cleavage, Cyclization) as Intermediate 4 on page (14-15). 3 mmol rink amide AM resin afforded (1.15 g, 85.8% purity) as a white solid. LCMS: RT=1.59 min, MS cal.: 1529.74, [M+H]⁺=1530.6, [M/2+H]⁺=765.4.

### Preparation of Intermediate 10:

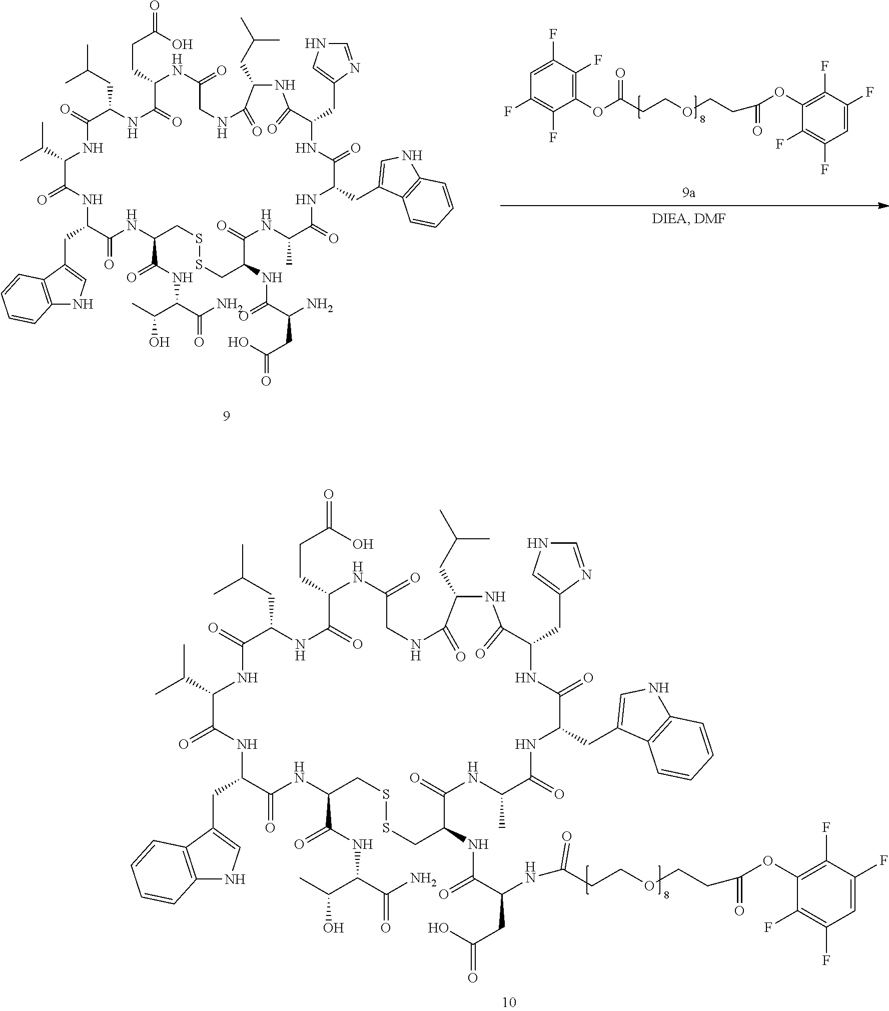

To a mixture of Intermediate 9a (501.15 mg, 653.71 μmol, 5.00 eq) in DMF (5 mL) was added a mixture of Intermediate 9 (200.0 mg, 130.74 μmol, 1.00 eq), DIEA (84.49 mg, 653.71 μmol, 113.86 uL, 5.00 eq) in DMF (3 mL) at 0° C. The reaction mixture was then stirred at 20° C. for 0.5h. The mixture was purified by prep-HPLC (TFA conditions) to afford Intermediate 10 (151 mg, 95.1% purity, 51.5% yield). LCMS: RT=1.10 min, MS cal.: 2130.29, [M/2+H]⁺=1065.51, [M/2+H]⁺=1082.14.

### Preparation of Intermediate 11:

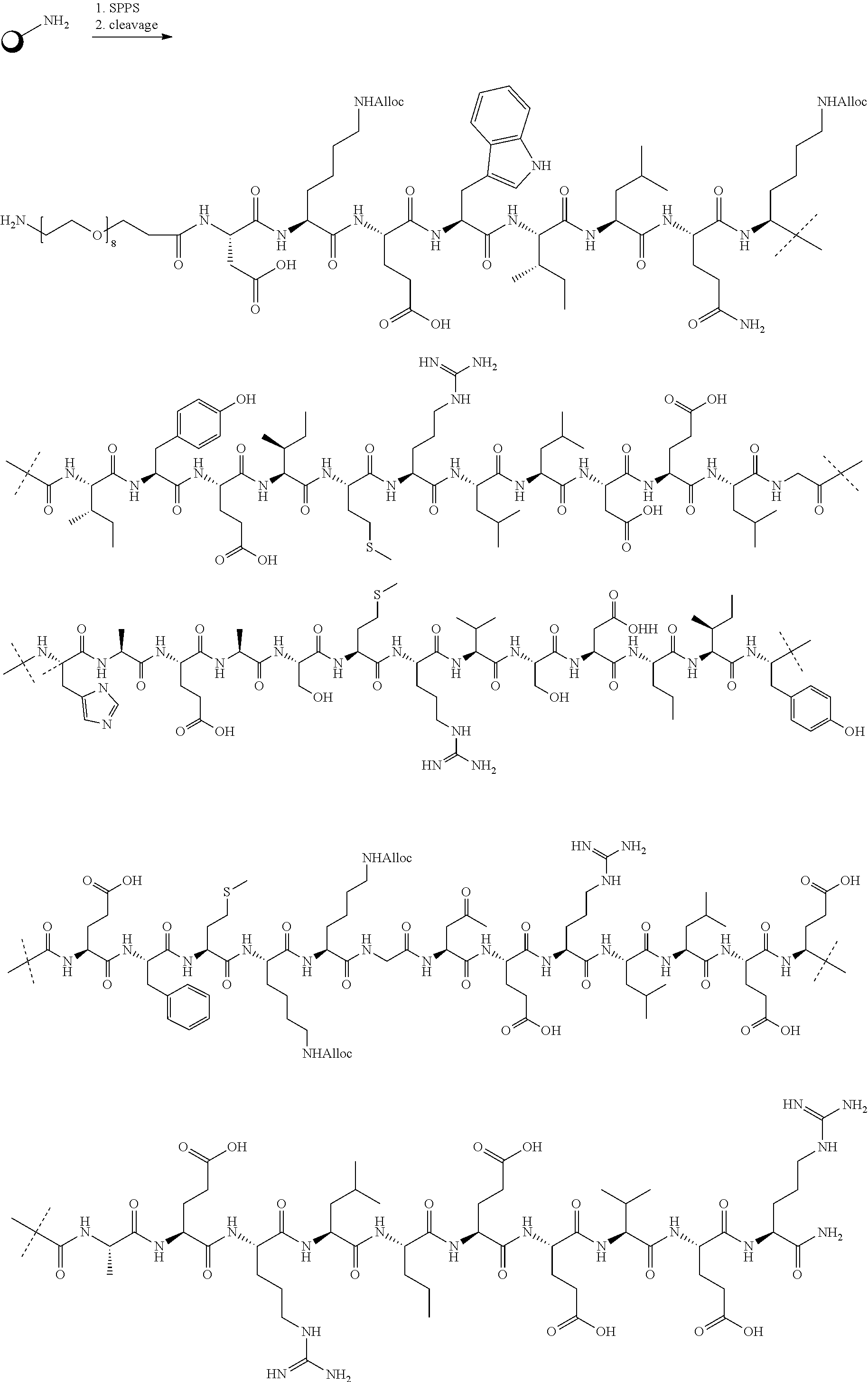

Intermediate 11 was synthesized using the same procedure (SPPS, Cleavage) as Intermediate 4 on page (14-15). 0.5 mmol rink amide AM resin afforded (200 mg, 85.8% purity, 4.53% yield) as a white solid. LCMS: RT=1.40 min, MS cal.: 7569.55, [M/6+H]⁺=1262.43, [M/7+H]⁺=1082.14.

### Preparation of Intermediate 12:

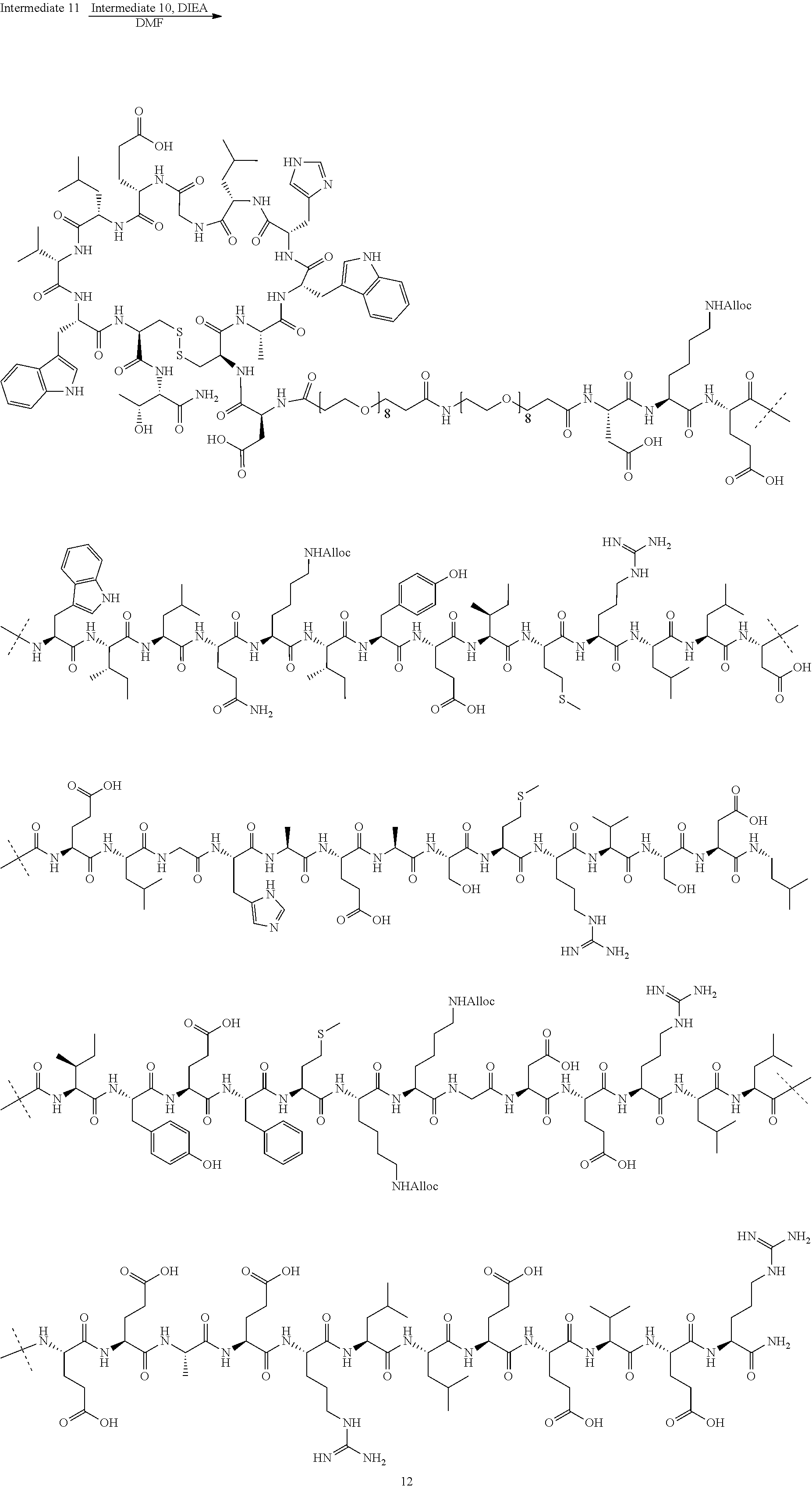

A mixture of Intermediate 10 (35.0 mg, 4.62 μmol, 1.00 eq), Intermediate 11 (29.55 mg, 13.87 μmol, 3.00 eq), DIEA (2.99 mg, 23.12 μmol, 4.03 μL, 5.00 eq) in DMSO (0.5 mL) was stirred at 20° C. for 1 h. Then the mixture was purified by prep-HPLC (TFA conditions) to afford Intermediate 12 (15.0 mg, 91.1% purity, 31.0% yield) as a white solid. LCMS: RT=1.30 min, MS cal.: 9533.76, [M/5+H]⁺=1907.40, [M/6+H]⁺=1589.80, [M/7+H]⁺=1362.70.

### Preparation of Compound 128:

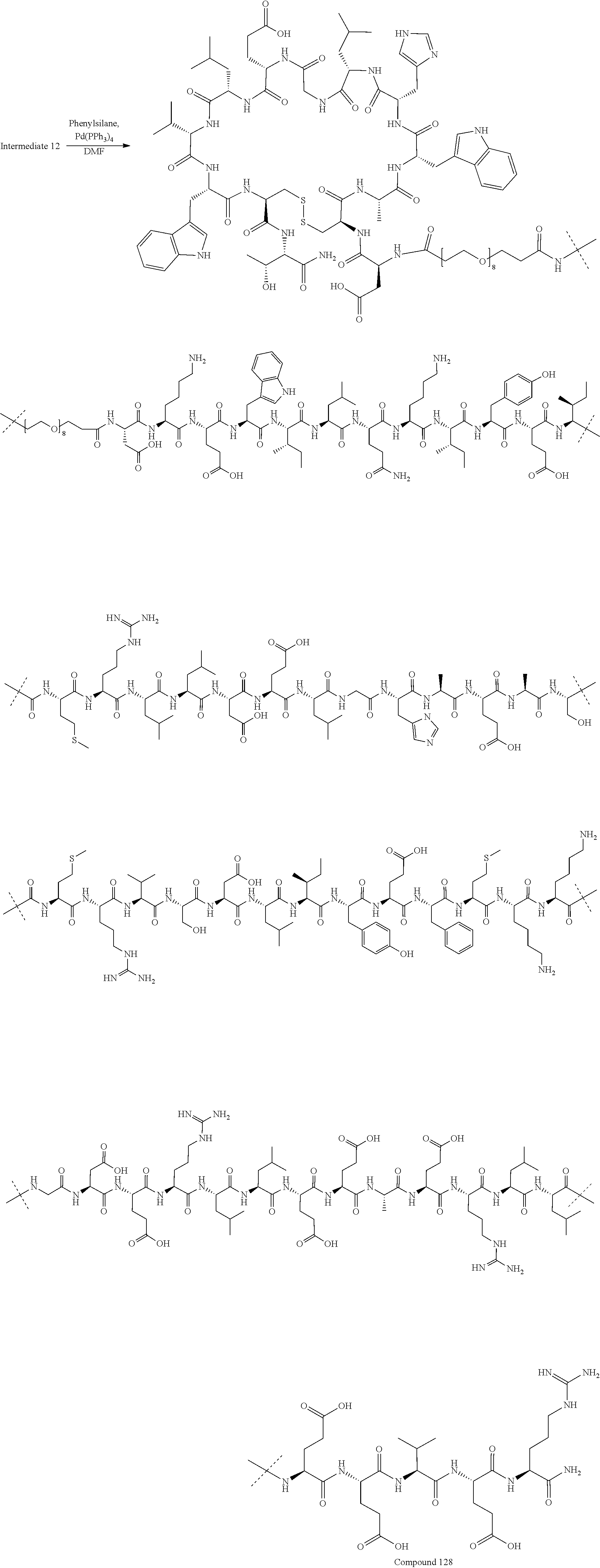

A mixture of Intermediate 12 (15.0 mg, 1.57 μmol, 1.00 eq), Pd(PPh₃)₄ (909.0 μg, 7.87e-1μμ, 0.50 eq), phenylsilane (1.70 mg, 15.73μ, 1.94 μL, 10.00 eq) in DMF (1 mL) was stirred at 20° C. for 1 hr. The mixture was purified by prep-HPLC (TFA conditions) to afford Compound 128 (5.5 mg, 78.2% purity, 29.72% yield) as a white solid. LCMS: RT=1.14 min, MS cal.: 9197.47, [M/5+H]⁺=1840.12, [M/6+H]⁺=1533.65, [M/7+H]⁺=1314.56.

### Example 5. Procedure for Preparation of Compound 129 (Linear Peptide+uABT)

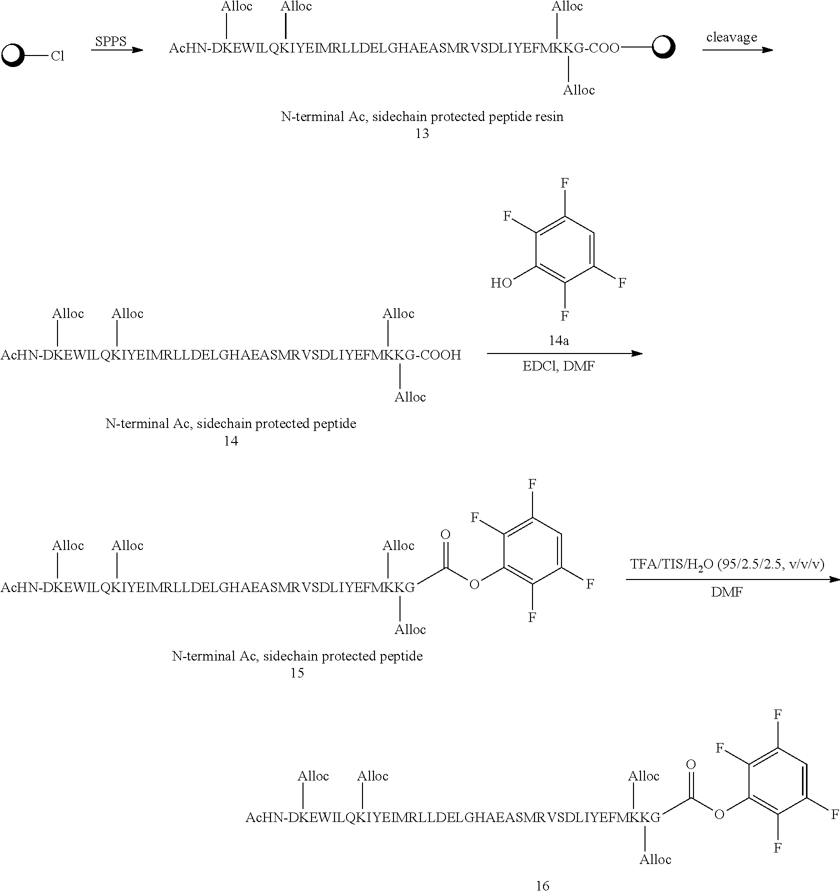

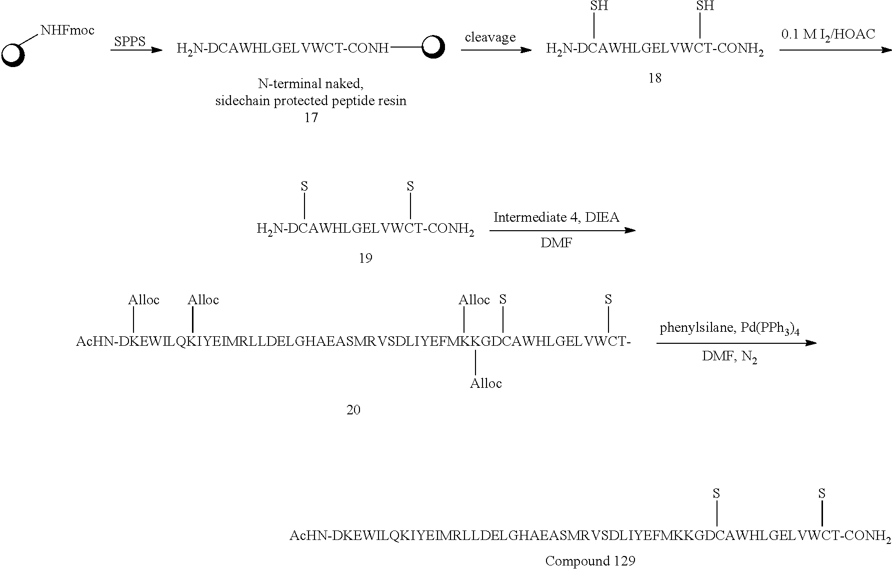

### Preparation of Intermediate 2 (Resin-Bound Peptide).

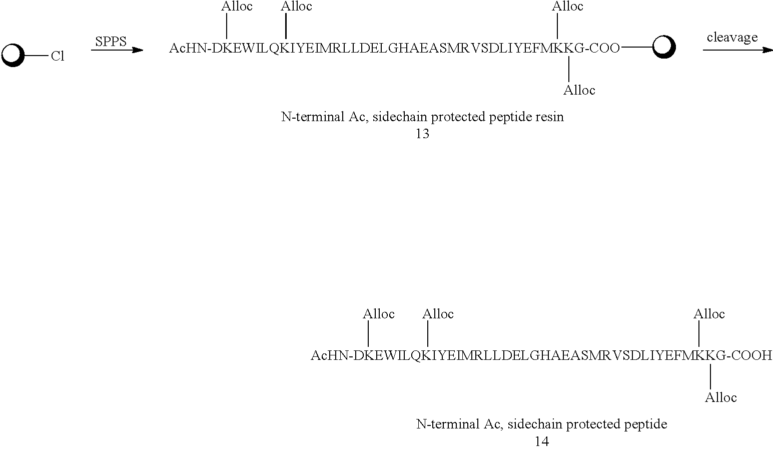

Peptide was synthesized using standard Fmoc chemistry (CTC resin).

Resin preparation: To the vessel containing CTC Resin (2.00 g, 2.00 mmol, 1.00 mmol/g) and Fmoc-Gly-OH (595.0 mg, 2.00 mmol, 1.00 equiv.) in DCM (20 mL) was added DIEA (4.00 equiv.) dropwise and mixed for 2 h with N₂ bubbling at 20° C. Then MeOH (2.0 mL) was added and bubbled with N₂ for another 30 min. The resin was washed with DMF (40 mL)*5, followed by adding 20% piperidine in DMF (40 mL) and bubbled with N₂ for 30 mins at 20° C. for Fmoc deprotection. The mixture was filtered and the resin was washed with DMF (40 mL)*5 before proceeding to next step.

Coupling: A solution of Fmoc-Lys(Alloc)-OH (2.72 g, 3.00 equiv.), HBTU (2.16 g, 2.85 equiv.) in DMF (20 mL) was added to the resin with N₂ bubbling. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (40 mL)*5.

Deprotection: 20% piperidine in DMF (40 mL) was added to the resin and the mixture was bubbled with N₂ for 30 mins at 20° C. The deprotection reaction was monitored by ninhydrin test, if it showed blue or brownish red, the reaction was completed. The resin was then washed with DMF (40 mL) *5.

Coupling and Deprotection steps were repeated for the following amino acids: Number #3-17, Table 3.

Coupling: A solution of Fmoc-Ala-OH (1.87 g, 3.00 equiv.), HATU (2.16 g, 2.85 equiv.) in DMF (20 mL) was added to the resin with N₂ bubbling for 30 mins at 20° C. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (60 mL)*5.

Deprotection step was repeated for Fmoc deprotection.

The coupling and deprotection steps were repeated for the following amino acids elongation: Number #19-39, TABLE 3.

Acetylation: A solution of Ac₂O/NMM/DMF (2/1/17, v/v/v, 40 mL) was added to resin, the mixture was bubbled with N₂ for 20 min. The acetylation reaction was monitored by ninhydrin test. The resin was then washed with DMF (40 mL)*5, MeOH (40 mL)*3, then dried under reduced pressure.

TABLE 3
The list of amino acids and the corresponding reagents used on SPPS.

| # | Materials | Coupling reagents |
|---|---|---|
| 1 | Fmoc-Gly-OH (1.00 equiv.) | DIEA (4.00 equiv.) |
| 2 | Fmoc-Lys(Alloc)-OH | HBTU (2.85 equiv.) and |
| | (3.00 equiv.) | DIEA (6.00 equiv.) |
| 3 | Fmoc-Lys(Alloc)-OH | HBTU (2.85 equiv.) and |
| | (3.00 equiv.) | DIEA (6.00 equiv.) |
| 4 | Fmoc-Met-OH | HBTU (2.85 equiv.) and |
| | (3.00 equiv.) | DIEA (6.00 equiv.) |
| 5 | Fmoc-Phe-OH | HBTU (2.85 equiv.) and |
| | (3.00 equiv.) | DIEA (6.00 equiv.) |
| 6 | Fmoc-Glu(OtBu)-OH | HBTU (2.85 equiv.) and |
| | (3.00 equiv.) | DIEA (6.00 equiv.) |
| 7 | Fmoc-Tyr(tBu)-OH | HBTU (2.85 equiv.) and |
| | (3.00 equiv.) | DIEA (6.00 equiv.) |
| 8 | Fmoc-Ile-OH | HBTU (2.85 equiv.) and |
| | (3.00 equiv.) | DIEA (6.00 equiv.) |
| 9 | Fmoc-Leu-OH | HBTU (2.85 equiv.) and |
| | (3.00 equiv.) | DIEA (6.00 equiv.) |
| 10 | Fmoc-Asp(OtBu)-OH | HBTU (2.85 equiv.) and |
| | (3.00 equiv.) | DIEA (6.00 equiv.) |
| 11 | Fmoc-Ser(tBu)-OH | HBTU (2.85 equiv.) and |
| | (3.00 equiv.) | DIEA (6.00 equiv.) |
| 12 | Fmoc-Val-OH | HBTU (2.85 equiv.) and |
| | (3.00 equiv.) | DIEA (6.00 equiv.) |
| 13 | Fmoc-Arg(Pbf)-OH | HBTU (2.85 equiv.) and |
| | (3.00 equiv.) | DIEA (6.00 equiv.) |
| 14 | Fmoc-Met-OH | HBTU (2.85 equiv.) and |
| | (3.00 equiv.) | DIEA (6.00 equiv.) |
| 15 | Fmoc-Ser(tBu)-OH | HBTU (2.85 equiv.) and |
| | (3.00 equiv.) | DIEA (6.00 equiv.) |
| 16 | Fmoc-Ala-OH | HBTU (2.85 equiv.) and |
| | (3.00 equiv.) | DIEA (6.00 equiv.) |
| 17 | Fmoc-Glu(OtBu)-OH | HBTU (2.85 equiv.) and |
| | (3.00 equiv.) | DIEA (6.00 equiv.) |
| 18 | Fmoc-Ala-OH | HATU (2.85 equiv.) and |
| | (3.00 equiv.) | DIEA (6.00 equiv.) |
| 19 | Fmoc-His(Trt)-OH | HATU (2.85 equiv.) and |
| | (3.00 equiv.) | DIEA (6.00 equiv.) |
| 20 | Fmoc-Gly-OH | HATU (2.85 equiv.) and |
| | (3.00 equiv.) | DIEA (6.00 equiv.) |
| 21 | Fmoc-Leu-OH | HATU (2.85 equiv.) and |
| | (3.00 equiv.) | DIEA (6.00 equiv.) |
| 22 | Fmoc-Glu(OtBu)-OH | HATU (2.85 equiv.) and |
| | (3.00 equiv.) | DIEA (6.00 equiv.) |
| 23 | Fmoc-Asp(OtBu)-OH | HATU (2.85 equiv.) and |
| | (3.00 equiv.) | DIEA (6.00 equiv.) |
| 24 | Fmoc-Leu-OH | HATU (2.85 equiv.) and |
| | (3.00 equiv.) | DIEA (6.00 equiv.) |
| 25 | Fmoc-Leu-OH | HATU (2.85 equiv.) and |
| | (3.00 equiv.) | DIEA (6.00 equiv.) |
| 26 | Fmoc-Arg(Pbf)-OH | HATU (2.85 equiv.) and |
| | (3.00 equiv.) | DIEA (6.00 equiv.) |
| 27 | Fmoc-Met-OH | HATU (2.85 equiv.) and |
| | (3.00 equiv.) | DIEA (6.00 equiv.) |
| 28 | Fmoc-Ile-OH | HATU (2.85 equiv.) and |
| | (3.00 equiv.) | DIEA (6.00 equiv.) |
| 29 | Fmoc-Glu(OtBu)-OH | HATU (2.85 equiv.) and |
| | (3.00 equiv.) | DIEA (6.00 equiv.) |
| 30 | Fmoc-Tyr(tBu)-OH | HATU (2.85 equiv.) and |
| | (3.00 equiv.) | DIEA (6.00 equiv.) |
| 31 | Fmoc-Ile-OH | HATU (2.85 equiv.) and |
| | (3.00 equiv.) | DIEA (6.00 equiv.) |
| 32 | Fmoc-Lys(Alloc)-OH | HATU (2.85 equiv.) and |
| | (3.00 equiv.) | DIEA (6.00 equiv.) |
| 33 | Fmoc-Gln(Trt)-OH | HATU (2.85 equiv.) and |
| | (3.00 equiv.) | DIEA (6.00 equiv.) |
| 34 | Fmoc-Leu-OH | HATU (2.85 equiv.) and |
| | (3.00 equiv.) | DIEA (6.00 equiv.) |
| 35 | Fmoc-Ile-OH | HATU (2.85 equiv.) and |
| | (3.00 equiv.) | DIEA (6.00 equiv.) |
| 36 | Fmoc-Trp-OH | HATU (2.85 equiv.) and |
| | (3.00 equiv.) | DIEA (6.00 equiv.) |
| 37 | Fmoc-Glu(OtBu)-OH | HATU (2.85 equiv.) and |
| | (3.00 equiv.) | DIEA (6.00 equiv.) |
| 38 | Fmoc-Lys(Alloc)-OH | HATU (2.85 equiv.) and |
| | (3.00 equiv.) | DIEA (6.00 equiv.) |
| 39 | Fmoc-Asp(OtBu)-OH | HATU (2.85 equiv.) and |
| | (3.00 equiv.) | DIEA (6.00 equiv.) |
| 40 | Acetylation | Ac2O/NMM/DMF |
| | | (2/1/17, v/v/v) |

Peptide Cleavage: Cleavage solution (HFIP/DCM, 1/4, v/v, 100 mL) was added to the flask containing the side-chain protected resin-bound peptide (intermediate 13 (SEQ ID NO: 12), 15.1 g, 2.00 mmol) at room temperature and stirred for 1 h. After filtration, the filtrate was collected. The filtrate was concentrated under reduced pressure. The residue was added MeCN/H₂O (4/6, v/v, 500 mL) and lyophilized to afford intermediate 14 (SEQ ID NO:12) (12.0 g, crude) as a white solid.

### Preparation of Intermediate 15

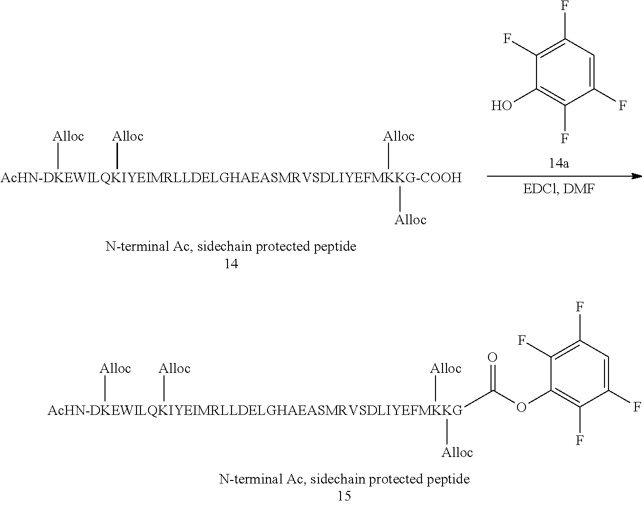

To a mixture of intermediate 14 (SEQ ID NO:12) (12.00 g, crude, 1.00 equiv.), intermediate 14a (3.32 g, 20.00 mmol, 10.00 equiv.) in DMF (100 mL) was added EDCI (2.30 g, 12 mmol, 6.00 equiv.) at 20° C. The reaction was stirred at 20° C. for 12 h. Then the mixture was precipitated with 0.1 M aq. HCl (cold, 1000 mL). After filtration, the solid was dried by lyophilization to afford intermediate 15 (SEQ ID NO:13) (18.30 g, crude) as a white solid.

### Preparation of Intermediate 16 (SEQ ID NO:43)

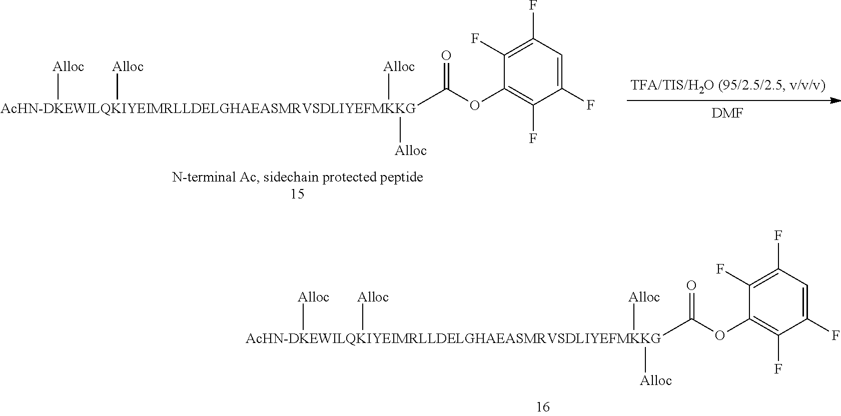

A solution of intermediate 15 (SEQ ID NO:13) (18.30 g, crude) in TFA/TIS/H₂O (95/2.5/2.5, v/v/v, 200 mL) was stirred at 20° C. for 2 h. The mixture was precipitated with cold isopropyl ether (800 mL). After filtration, the solid was washed with isopropyl ether (500 mL) twice, dried under reduced pressure at room temperature. The solid was purified by prep-HPLC (acidic condition, A: 0.075% TFA/H₂O; B: MeCN), followed by lyophilization to afford intermediate 16 (SEQ ID NO:43) (1.08 g, 74.0% purity, 7.64% yield, containing peptide with C-terminal free that TFP hydrolyzed) as a white solid. LCMS: RT=1.34 min, MS cal.: 5226.85, mass observed: [M+3H]³⁺=1743.14, [M+4H]⁴⁺+=1307.75.

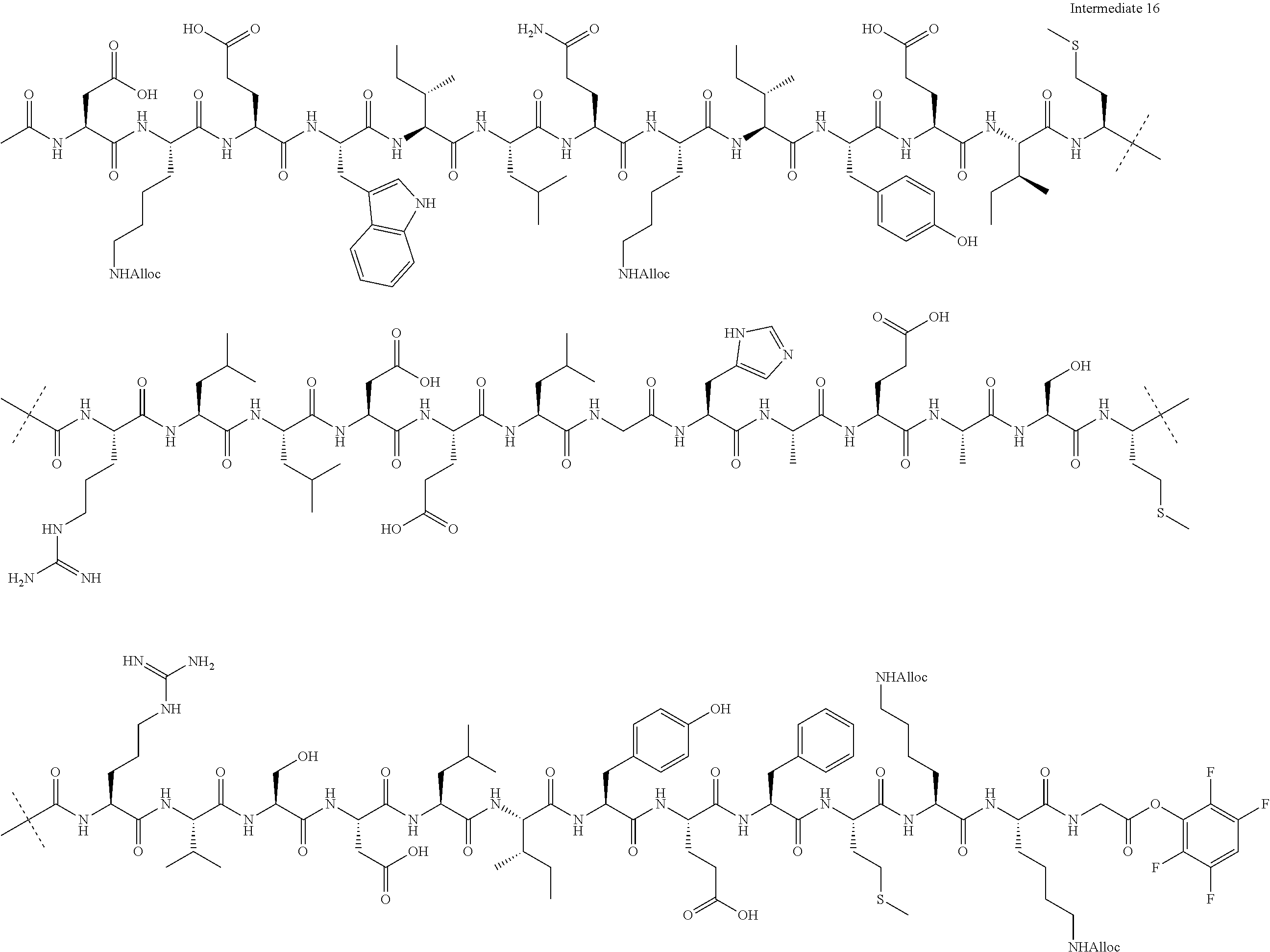

Preparation of Intermediate 19 (uABT)

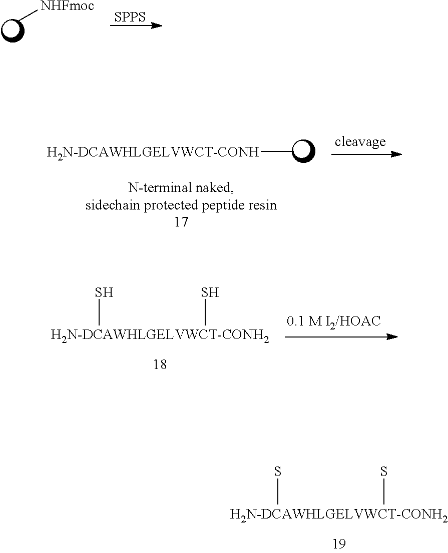

Peptide was synthesized using standard Fmoc chemistry (Rink AM resin).

Resin preparation: To the vessel containing Rink Amide AM resin (6.25 g, 2.00 mmol, 0.32 mmol/g) and DMF (80 mL) was bubbled with N₂ for 2 h at 20° C. Then 20% piperidine in DMF (80 mL) was added and the mixture was bubbled with N₂ for 30 min at 20° C. The mixture was filtered and washed with DMF (40 mL)*5 before proceeding to next step.

Coupling: A solution of Fmoc-Thr(tBu)-OH (2.38 g, 6.00 mmol, 3.00 equiv.), HBTU (2.19 g, 5.70 mmol, 2.85 equiv.) in DMF (40 mL) was added to the resin with N₂ bubbling. Then DIEA (1.54 g, 12.00 mmol, 6.00 equiv.) was added to the mixture dropwise and bubbled with N₂ for 30 min at 20° C. The coupling reaction was monitored by ninhydrin test. The resin was then washed with DMF (80 mL)*5.

Deprotection: 20% piperidine in DMF (80 mL) was added to the resin and the mixture was bubbled with N₂ for 30 min at 20° C. The deprotection reaction was monitored by ninhydrin test. The resin was then washed with DMF (80 mL)*5.

Coupling and Deprotection steps were repeated for the following amino acids elongation: Number #2-12, Table 4.

After all the steps were completed, the resin was washed with DMF (80 mL)*5, MeOH (80 mL)*5, then dried under reduced pressure to afford resin-bound peptide intermediate 19 (Rink AM resin, 12.72 g, 2.00 mmol).

TABLE 4

| # | Materials | Coupling reagents |
|---|---|---|
| 1 | Fmoc-Thr(OtBu)-OH (3.00 equiv.) | HBTU (2.85 equiv.) and DIEA (6.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-His(Trt)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |

Peptide Cleavage, Cyclization and Purification: Cleavage solution (TFA/TIS/H₂O, 95/2.5/2.5, v/v/v, 240 mL) was added to the flask containing the side-chain protected resin-bound peptide intermediate 17 [(SEQ ID NO:14), protected], (Rink AM resin, 12.72 g, 2.00 mmol) at room temperature and stirred for 2 h. After filtration, the filtrate was collected.

The filtrate was precipitated with cold isopropyl ether (1.5 L). After filtration, the solid was washed with isopropyl ether (500 mL) twice, and the crude peptide was dried under reduced pressure for 2 h to afford intermediate 18 (SEQ ID NO:33)(4.52 g, crude) as a white solid. To a mixture of intermediate 18 (4.52 g, crude) in HOAc/MeCN/H₂O (4/3/3, v/v/v, 2.0 L) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (0.075% TFA/H₂O, MeCN), followed by lyophilization to afford intermediate 19 (SEQ ID NO:15) (688.0 mg, 85.8% purity, 22.5% yield) as a white solid. LCMS: RT=1.60 min, MS cal.: 1529.74, mass observed: [M+H]⁺=1530.6, [M+2H]²⁺=765.9.

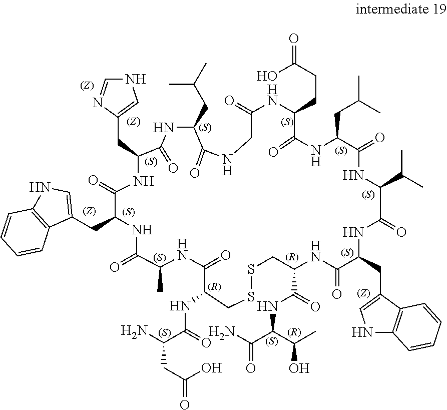

### Preparation of Intermediate 20 (Fragments Coupling)

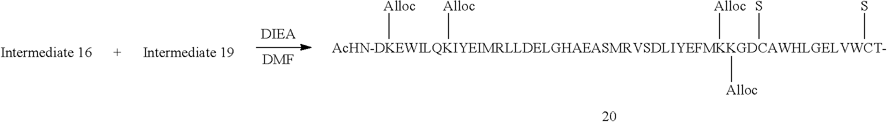

To a mixture of intermediate 16 (SEQ ID NO:43) (300.0 mg, 57.4 mol, 1.00 equiv.) and intermediate 19 (SEQ ID NO:15) (87.8 mg, 57.4 mol, 1.00 equiv.) in DMF (3 mL) was added DIEA (74.1 mg, 573.9 mol, 10.0 equiv.) at 20° C. The mixture was stirred at 20° C. for 2 h. Monitoring LCMS found intermediate 19 was consumed completely. The mixture was acidified by 1 M HCl to pH=5, purified by prep-HPLC (A: 0.075% TFA/H₂O, B: MeCN) directly, then lyophilized to afford intermediate 20 (SEQ ID NO:16) (120.0 mg, 97.6% purity, 31.7% yield) as a white solid. TOF: RT=2.18 min, MS cal.: 6590.51, mass observed: [M+4H]⁴⁺=1648.33, [M+5H]⁵⁺=1318.66, [M+6H]⁶⁺=1099.05.

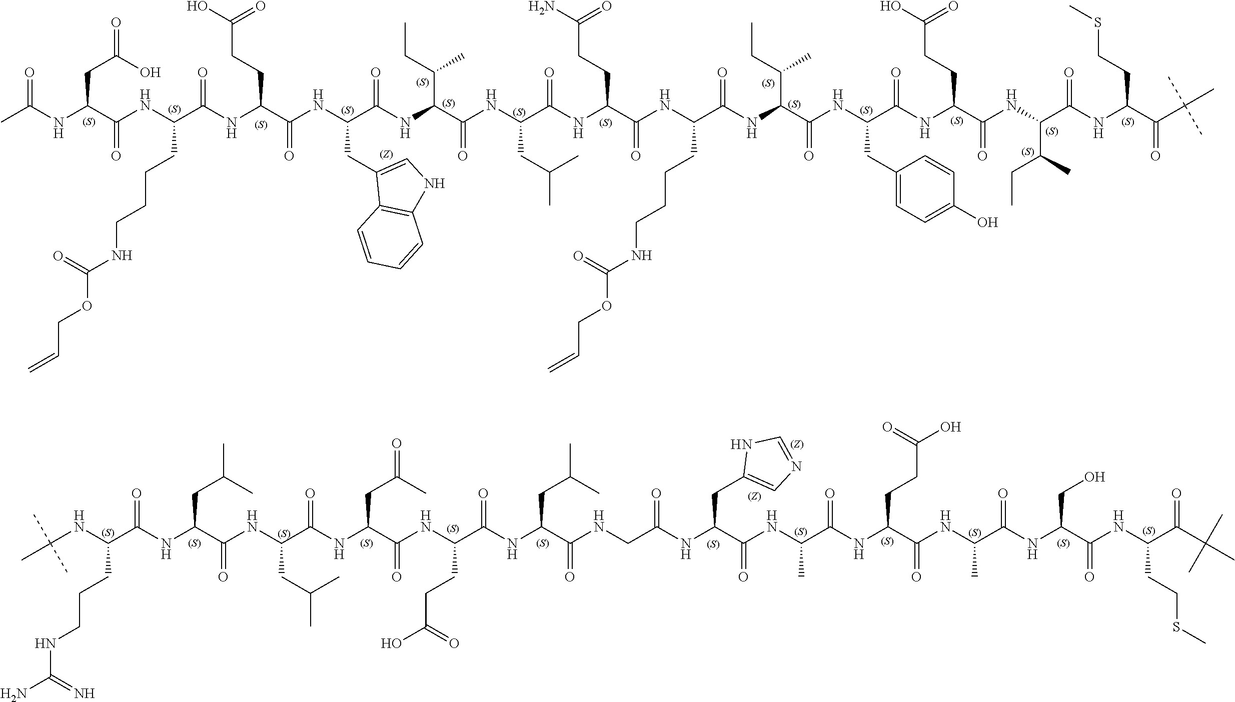

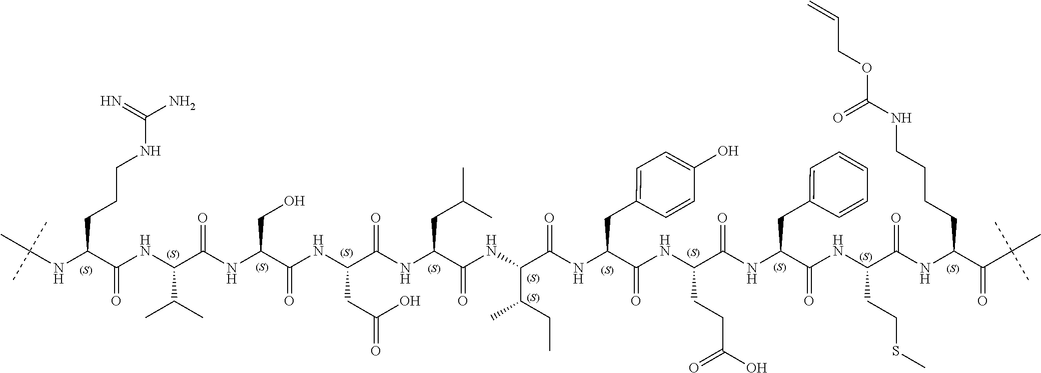

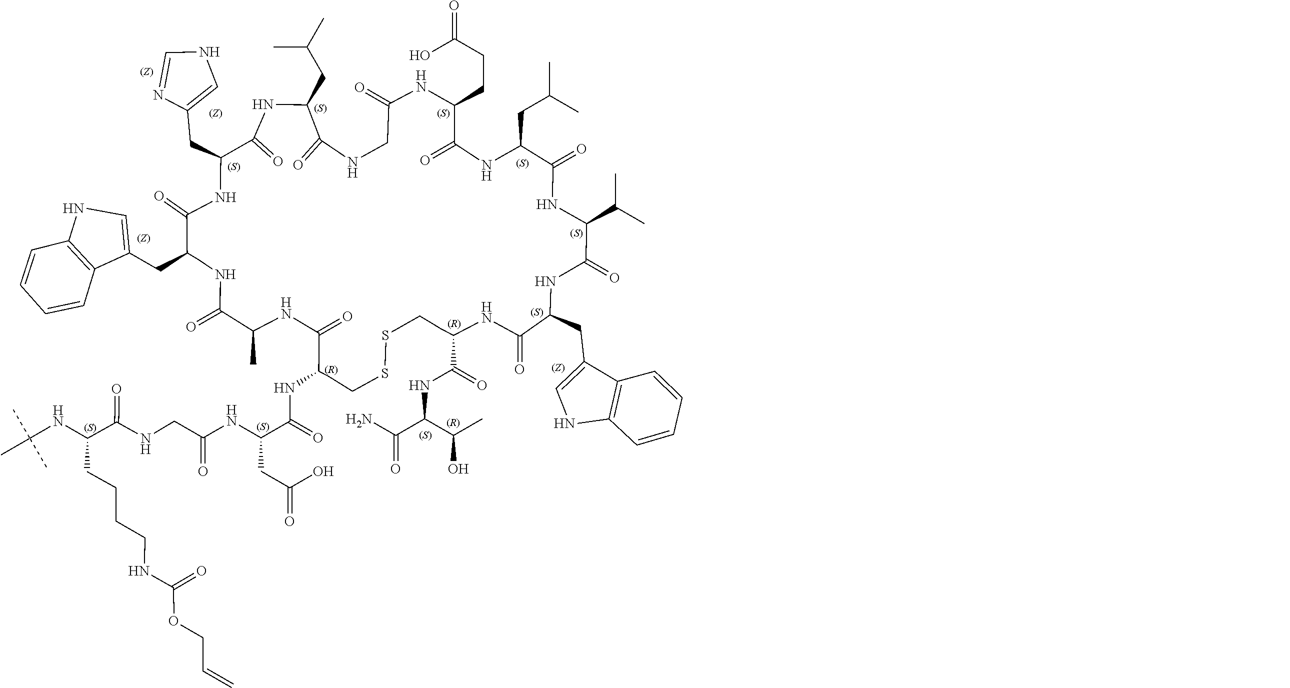

Preparation of Compound 129 (de-Alloc)

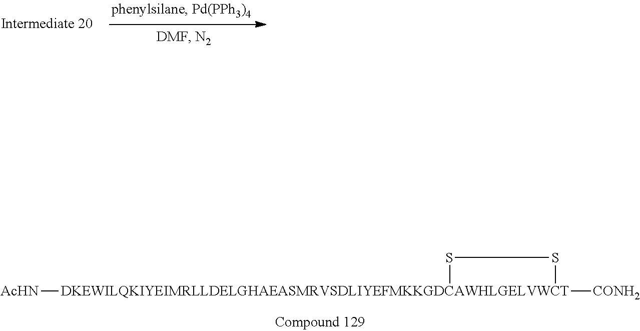

Intermediate 20 (120.0 mg, 18.2 mol, 1.00 equiv.), phenylsilane (19.6 mg, 182.00 mol, 21.8 uL, 10.00 equiv.), Pd(PPh₃)₄ (10.5 mg, 9.10 μmol, 0.50 equiv.) in DMF (1 mL) was stirred under N₂ atmosphere at 20° C. for 0.5 h. The mixture was purified by prep-HPLC (A: 0.075% TFA/H₂O, B: MeCN) directly, then lyophilized to afford Compound 129 (SEQ ID NO:17) (29.9 mg, 94.5% purity, 24.8% yield) as a white solid. LCMS: RT=1.07 min, MS cal.: 6254.22, mass observed: [M+4H]⁴⁺=1564.53, [M+5H]⁵⁺=1251.29, [M+6H]⁶⁺=1043.20.

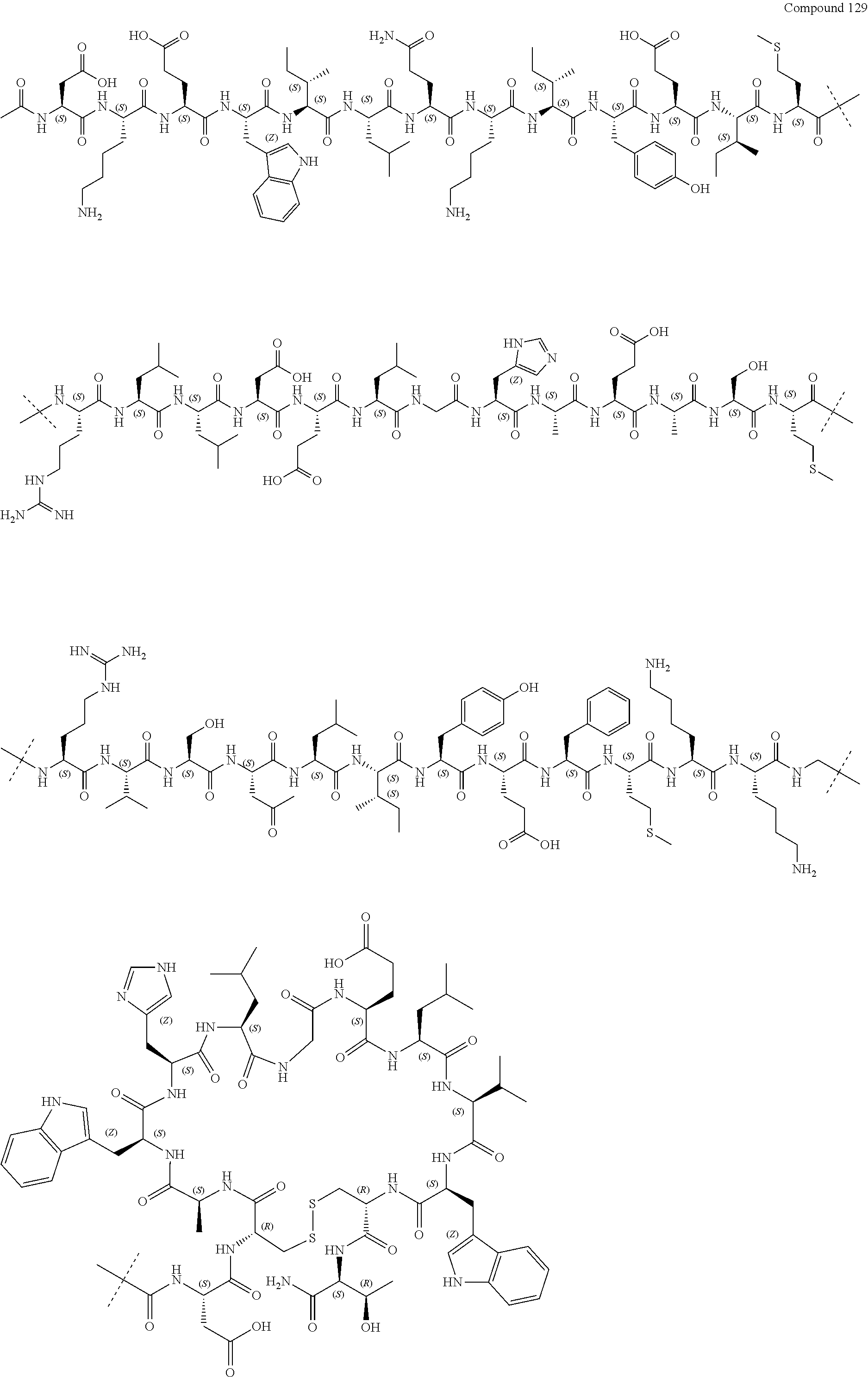

### Example 6. Procedure for Preparation of Compound 130

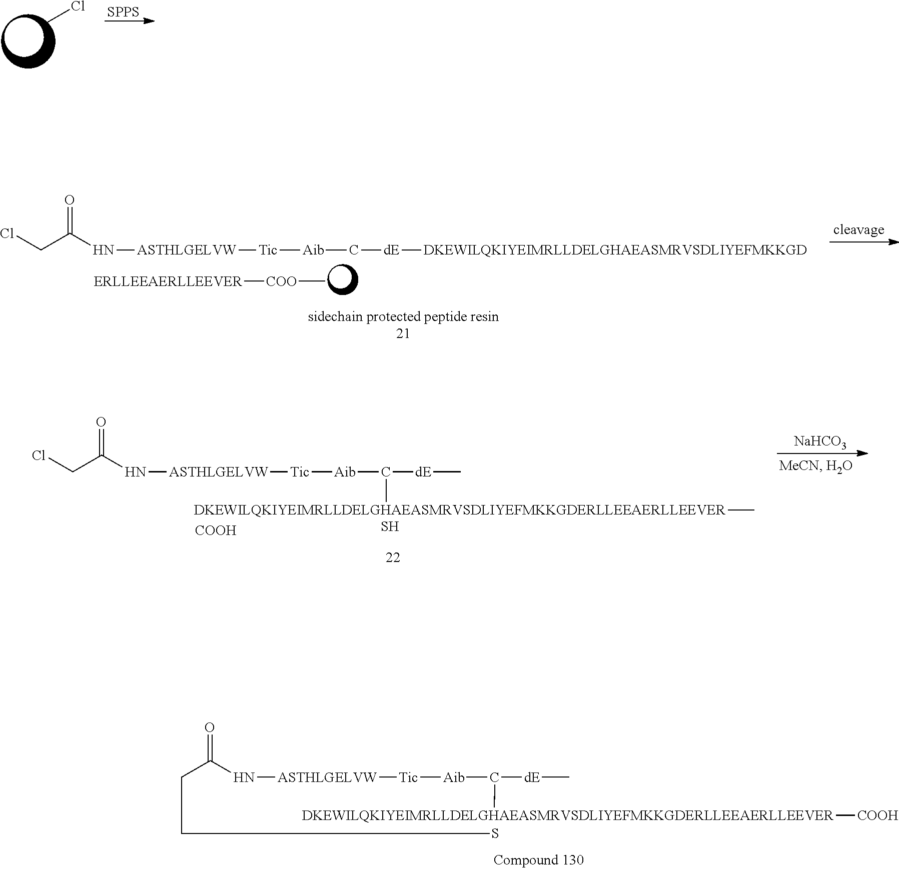

Peptide was synthesized using standard Fmoc chemistry (CTC resin).

Resin preparation: To the vessel containing CTC Resin (0.50 g, 0.50 mmol, 1.00 mmol/g) and Fmoc-Arg(Pbf)-OH (324.0 mg, 0.50 mmol, 1.00 equiv.) in DCM (5 mL) was added DIEA (4.00 equiv.) dropwise and mixed for 2 h with N₂ bubbling at 20° C. Then MeOH (0.5 mL) was added and bubbled with N₂ for another 30 min. The resin was washed with DMF (10 mL)*5, followed by adding 20% piperidine in DMF (10 mL) and bubbled with N₂ for 30 mins at 20° C. for Fmoc deprotection. The mixture was filtered and the resin was washed with DMF (10 mL)*5 before proceeding to next step.

Coupling: A solution of Fmoc-Glu(OtBu)-OH (638.20 mg, 1.50 mmol, 3.00 equiv.), HBTU (546.7 mg, 1.42 mmol, 2.85 equiv.) in DMF (5 mL) was added to the resin with N₂ bubbling. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (10 mL)*5.

Deprotection: 20% piperidine in DMF (10 mL) was added to the resin and the mixture was bubbled with N₂ for 30 mins at 20° C. The deprotection reaction was monitored by ninhydrin test, if it showed blue or brownish red, the reaction was completed. The resin was then washed with DMF (10 mL) *5.

Steps 2 and 3 were repeated for the following amino acids elongation: Number #3-18, Table 5.

Coupling: A solution of Fmoc-Lys(Boc)-OH (702.0 mg, 3.00 equiv.), HATU (548.6 mg, 2.85 equiv.) in DMF (5 mL) was added to the resin with N₂ bubbling for 30 mins at 20° C. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (10 mL)*5.

Step 3 was repeated for Fmoc deprotection.

Steps 5 and 6 were repeated for the following amino acids: Number #19-70, Table 5.

Coupling N-(Chloroacetoxy)succinimide: A solution of N-(Chloroacetoxy)succinimide (573.0 mg, 6.00 equiv.), DIEA (774.0 mg, 12.00 equiv.) in DMF (5 mL) was added to resin, the mixture was bubbled with N₂ for 20 min. The acetylation reaction was monitored by ninhydrin test. The resin was then washed with DMF (40 mL)*5, MeOH (40 mL)*3, then dried under reduced pressure to afford intermediate 21 (SEQ ID NO:19) (CTC resin, 7.50 g, 0.50 mmol).

TABLE 5
The list of amino acids and the corresponding reagents used on SPPS.

| # | Materials | Coupling reagents |
|---|---|---|
| 1 | Fmoc-Arg(Pbf)-OH (1.00 equiv.) | DIEA (4.00 equiv.) |
| 2 | Fmoc-Glu(OtBu)-OH (3.00 equiv.) | HBTU (2.85 equiv.) and DIEA (6.00 equiv.) |
| 3 | Fmoc-Val-OH (3.00 equiv.) | HBTU (2.85 equiv.) and DIEA (6.00 equiv.) |
| 4 | Fmoc-Glu(OtBu)-OH (3.00 equiv.) | HBTU (2.85 equiv.) and DIEA (6.00 equiv.) |
| 5 | Fmoc-Glu(OtBu)-OH (3.00 equiv.) | HBTU (2.85 equiv.) and DIEA (6.00 equiv.) |
| 6 | Fmoc-Leu-OH (3.00 equiv.) | HBTU (2.85 equiv.) and DIEA (6.00 equiv.) |
| 7 | Fmoc-Leu-OH (3.00 equiv.) | HBTU (2.85 equiv.) and DIEA (6.00 equiv.) |
| 8 | Fmoc-Arg(Pbf)-OH (3.00 equiv.) | HBTU (2.85 equiv.) and DIEA (6.00 equiv.) |
| 9 | Fmoc-Glu(OtBu)-OH (3.00 equiv.) | HBTU (2.85 equiv.) and DIEA (6.00 equiv.) |
| 10 | Fmoc-Ala-OH (3.00 equiv.) | HBTU (2.85 equiv.) and DIEA (6.00 equiv.) |
| 11 | Fmoc-Glu(OtBu)-OH (3.00 equiv.) | HBTU (2.85 equiv.) and DIEA (6.00 equiv.) |
| 12 | Fmoc-Glu(OtBu)-OH (3.00 equiv.) | HBTU (2.85 equiv.) and DIEA (6.00 equiv.) |
| 13 | Fmoc-Leu-OH (3.00 equiv.) | HBTU (2.85 equiv.) and DIEA (6.00 equiv.) |
| 14 | Fmoc-Leu-OH (3.00 equiv.) | HBTU (2.85 equiv.) and DIEA (6.00 equiv.) |
| 15 | Fmoc-Arg(Pbf)-OH (3.00 equiv.) | HBTU (2.85 equiv.) and DIEA (6.00 equiv.) |
| 16 | Fmoc-Glu(OtBu)-OH (3.00 equiv.) | HBTU (2.85 equiv.) and DIEA (6.00 equiv.) |
| 17 | Fmoc-Asp(OtBu)-OH (3.00 equiv.) | HBTU (2.85 equiv.) and DIEA (6.00 equiv.) |
| 18 | Fmoc-Gly-OH (3.00 equiv.) | HBTU (2.85 equiv.) and DIEA (6.00 equiv.) |
| 19 | Fmoc-Lys(Boc)-OH (3.00 equiv.) | HATU (2.85 equiv.) and DIEA (6.00 equiv.) |
| 20 | Fmoc-Lys(Boc)-OH (3.00 equiv.) | HATU (2.85 equiv.) and DIEA (6.00 equiv.) |
| 21 | Fmoc-Met-OH (3.00 equiv.) | HATU (2.85 equiv.) and DIEA (6.00 equiv.) |
| 22 | Fmoc-Phe-OH (3.00 equiv.) | HATU (2.85 equiv.) and DIEA (6.00 equiv.) |
| 23 | Fmoc-Glu(OtBu)-OH (3.00 equiv.) | HATU (2.85 equiv.) and DIEA (6.00 equiv.) |
| 24 | Fmoc-Tyr(tBu)-OH (3.00 equiv.) | HATU (2.85 equiv.) and DIEA (6.00 equiv.) |
| 25 | Fmoc-Ile-OH (3.00 equiv.) | HATU (2.85 equiv.) and DIEA (6.00 equiv.) |
| 26 | Fmoc-Leu-OH (3.00 equiv.) | HATU (2.85 equiv.) and DIEA (6.00 equiv.) |
| 27 | Fmoc-Asp(OtBu)-OH (3.00 equiv.) | HATU (2.85 equiv.) and DIEA (6.00 equiv.) |
| 28 | Fmoc-Ser(OtBu)-OH (3.00 equiv.) | HATU (2.85 equiv.) and DIEA (6.00 equiv.) |
| 29 | Fmoc-Val-OH (3.00 equiv.) | HATU (2.85 equiv.) and DIEA (6.00 equiv.) |
| 30 | Fmoc-Arg(Pbf)-OH (3.00 equiv.) | HATU (2.85 equiv.) and DIEA (6.00 equiv.) |
| 31 | Fmoc-Met-OH (3.00 equiv.) | HATU (2.85 equiv.) and DIEA (6.00 equiv.) |
| 32 | Fmoc-Ser(tBu)-OH (3.00 equiv.) | HATU (2.85 equiv.) and DIEA (6.00 equiv.) |
| 33 | Fmoc-Ala-OH (3.00 equiv.) | HATU (2.85 equiv.) and DIEA (6.00 equiv.) |
| 34 | Fmoc-Glu(OtBu)-OH (3.00 equiv.) | HATU (2.85 equiv.) and DIEA (6.00 equiv.) |
| 35 | Fmoc-Ala-OH (3.00 equiv.) | HATU (2.85 equiv.) and DIEA (6.00 equiv.) |
| 36 | Fmoc-His(Trt)-OH (3.00 equiv.) | HATU (2.85 equiv.) and DIEA (6.00 equiv.) |
| 37 | Fmoc-Gly-OH (3.00 equiv.) | HATU (2.85 equiv.) and DIEA (6.00 equiv.) |
| 38 | Fmoc-Leu-OH (3.00 equiv.) | HATU (2.85 equiv.) and DIEA (6.00 equiv.) |
| 39 | Fmoc-Glu(OtBu)-OH (3.00 equiv.) | HATU (2.85 equiv.) and DIEA (6.00 equiv.) |
| 40 | Fmoc-Asp(OtBu)-OH (3.00 equiv.) | HATU (2.85 equiv.) and DIEA (6.00 equiv.) |
| 41 | Fmoc-Leu-OH (3.00 equiv.) | HATU (2.85 equiv.) and DIEA (6.00 equiv.) |
| 42 | Fmoc-Leu-OH (3.00 equiv.) | HATU (2.85 equiv.) and DIEA (6.00 equiv.) |
| 43 | Fmoc-Arg(Pbf)-OH (3.00 equiv.) | HATU (2.85 equiv.) and DIEA (6.00 equiv.) |
| 44 | Fmoc-Met-OH (3.00 equiv.) | HATU (2.85 equiv.) and DIEA (6.00 equiv.) |
| 45 | Fmoc-Ile-OH (3.00 equiv.) | HATU (2.85 equiv.) and DIEA (6.00 equiv.) |
| 46 | Fmoc-Glu(OtBu)-OH (3.00 equiv.) | HATU (2.85 equiv.) and DIEA (6.00 equiv.) |
| 47 | Fmoc-Tyr(tBu)-OH (3.00 equiv.) | HATU (2.85 equiv.) and DIEA (6.00 equiv.) |
| 48 | Fmoc-Ile-OH (3.00 equiv.) | HATU (2.85 equiv.) and DIEA (6.00 equiv.) |
| 49 | Fmoc-Lys(Boc)-OH (3.00 equiv.) | HATU (2.85 equiv.) and DIEA (6.00 equiv.) |
| 50 | Fmoc-Gln(Trt)-OH (3.00 equiv.) | HATU (2.85 equiv.) and DIEA (6.00 equiv.) |
| 51 | Fmoc-Leu-OH (6.00 equiv.) | HATU (5.70 equiv.) and DIEA (12.00 equiv.) |
| 52 | Fmoc-Ile-OH (6.00 equiv.) | HATU (5.70 equiv.) and DIEA (12.00 equiv.) |
| 53 | Fmoc-Trp(Boc)-OH (6.00 equiv.) | HATU (5.70 equiv.) and DIEA (12.00 equiv.) |
| 54 | Fmoc-Glu(OtBu)-OH (6.00 equiv.) | HATU (5.70 equiv.) and DIEA (12.00 equiv.) |
| 55 | Fmoc-Lys(Boc)-OH (6.00 equiv.) | HATU (5.70 equiv.) and DIEA (12.00 equiv.) |
| 56 | Fmoc-Asp(OtBu)-OH (6.00 equiv.) | HATU (5.70 equiv.) and DIEA (12.00 equiv.) |
| 57 | Fmoc-D-Glu(OtBu)-OH (6.00 equiv.) | HATU (5.70 equiv.) and DIEA (12.00 equiv.) |
| 58 | Fmoc-Cys(Trt)-OH (6.00 equiv.) | HATU (5.70 equiv.) and DIEA (12.00 equiv.) |
| 59 | Fmoc-Aib-OH (6.00 equiv.) | HATU (5.70 equiv.) and DIEA (12.00 equiv.) |
| 60 | Fmoc-Tic-OH (6.00 equiv.) | HATU (5.70 equiv.) and DIEA (12.00 equiv.) |
| 61 | Fmoc-Trp(Boc)-OH (6.00 equiv.) | HATU (5.70 equiv.) and DIEA (12.00 equiv.) |
| 62 | Fmoc-Val-OH (6.00 equiv.) | HATU (5.70 equiv.) and DIEA (12.00 equiv.) |
| 63 | Fmoc-Leu-OH (6.00 equiv.) | HATU (5.70 equiv.) and DIEA (12.00 equiv.) |
| 64 | Fmoc-Glu(OtBu)-OH (6.00 equiv.) | HATU (5.70 equiv.) and DIEA (12.00 equiv.) |
| 65 | Fmoc-Gly-OH (6.00 equiv.) | HATU (5.70 equiv.) and DIEA (12.00 equiv.) |
| 66 | Fmoc-Leu-OH (6.00 equiv.) | HATU (5.70 equiv.) and DIEA (12.00 equiv.) |
| 67 | Fmoc-His(Trt)-OH (6.00 equiv.) | HATU (5.70 equiv.) and DIEA (12.00 equiv.) |
| 68 | Fmoc-Tyr(tBu)-OH (6.00 equiv.) | HATU (5.70 equiv.) and DIEA (12.00 equiv.) |
| 69 | Fmoc-Ser(tBu)-OH (6.00 equiv.) | HATU (5.70 equiv.) and DIEA (12.00 equiv.) |
| 70 | Fmoc-Ala-OH (6.00 equiv.) | HATU (5.70 equiv.) and DIEA (12.00 equiv.) |
| 71 | N-(Chloroacetoxy)succinimide (6.00 | DIEA (12.00 equiv.) |
| | equiv.) | |

Peptide cleavage, cyclization and purification: Cleavage solution (TFA/TIS/H₂O, 95/2.5/2.5, v/v/v, 20 mL) was added to the flask containing the side-chain protected resin-bound peptide (intermediate 21, 7.50 g, 0.50 mmol) at room temperature and stirred for 1 h.

After filtration, the filtrate was collected. The filtrate was precipitated with cold isopropyl ether (100 mL). After filtration, the solid was washed with isopropyl ether (50 mL) twice, and the crude peptide was dried under reduced pressure for 2 h to afford intermediate 22 (4.55 g, crude) as a white solid.

To a mixture of intermediate 22 (SEQ ID NO:34) (4.55 g, crude) in MeCN/H₂O (5/5, v/v, 500 mL) was added NaHCO₃ to pH=8, then the mixture was stirred at 20° C. for 5 h. After filtration, the filtrate was purified by prep-HPLC (0.075% TFA/H₂O, MeCN) directly, followed by lyophilization to afford Compound 130 (SEQ ID NO:20) (30.8 mg, 94.7% purity, 0.68% yield) as a white solid. LCMS: RT=2.34 min, MS cal.: 8483.60, mass observed: [M+5H]⁺=1697.60, [M+6H]⁶⁺=1415.40, [M+7H]⁷=1212.70.

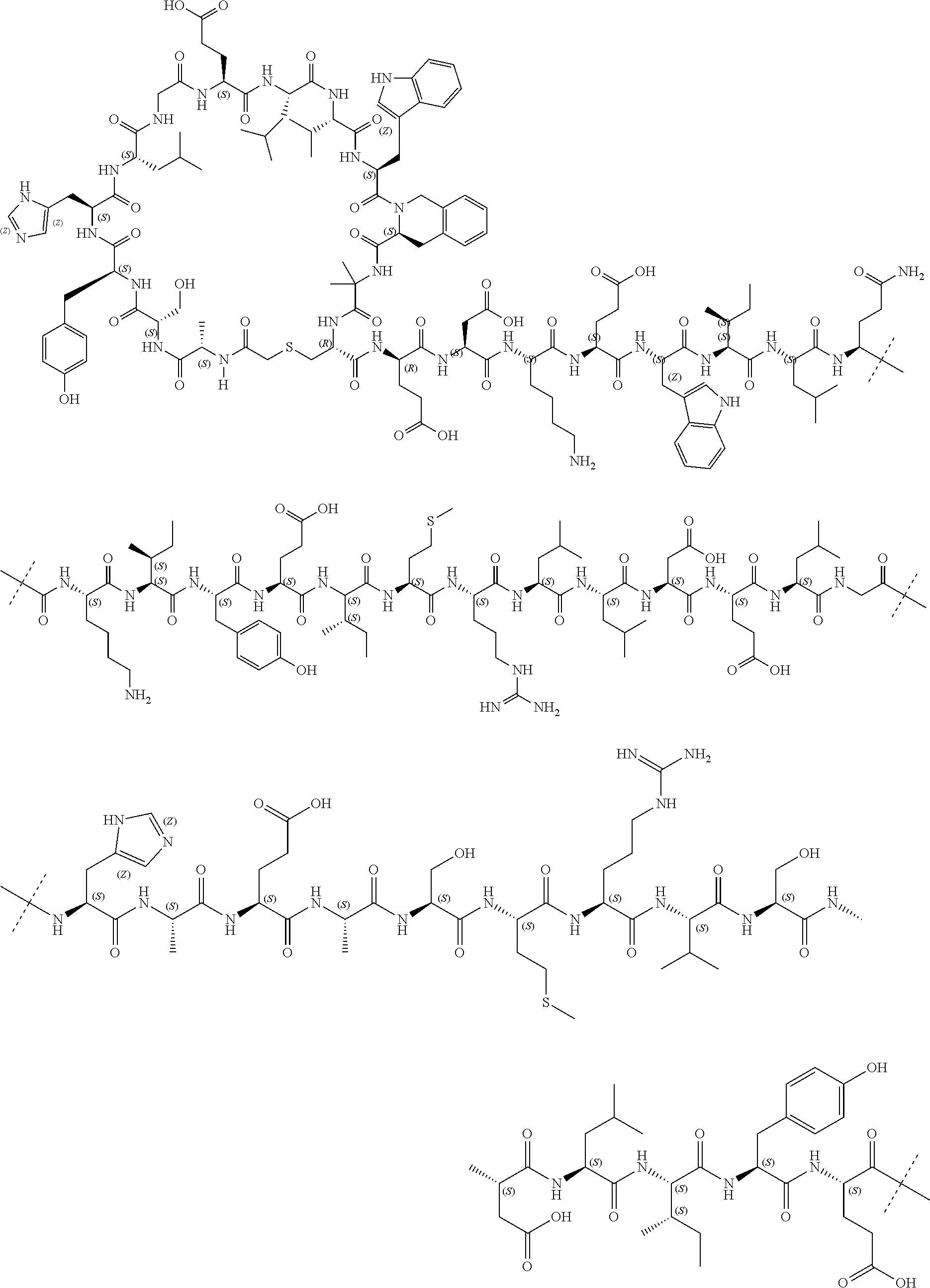

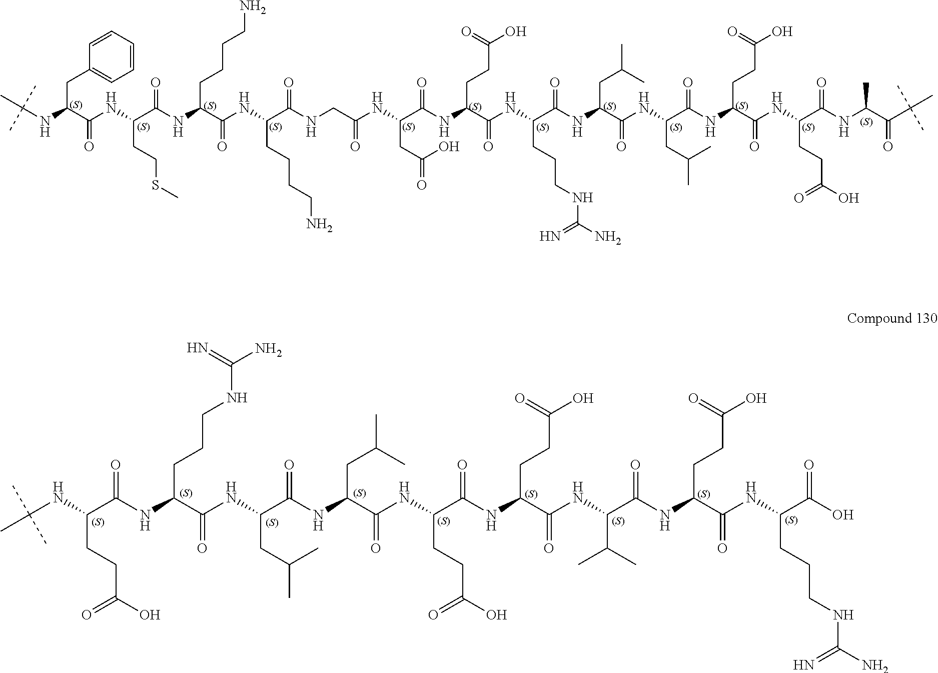

Compound 131 was synthesized using the same method as Compound 130 (SEQ ID NO:20). 0.50 mmol resin afforded Compound 131 (21.4 mg, 92.3% purity, 0.76% yield) as a white solid. LCMS: RT=2.52 min, MS cal.: 5757.68, [M+3H]³⁺=1919.60, [M+4H]⁴⁺+=1440.20, [M+5H]⁵⁺=1152.30, [M+6H]⁶⁺=960.40

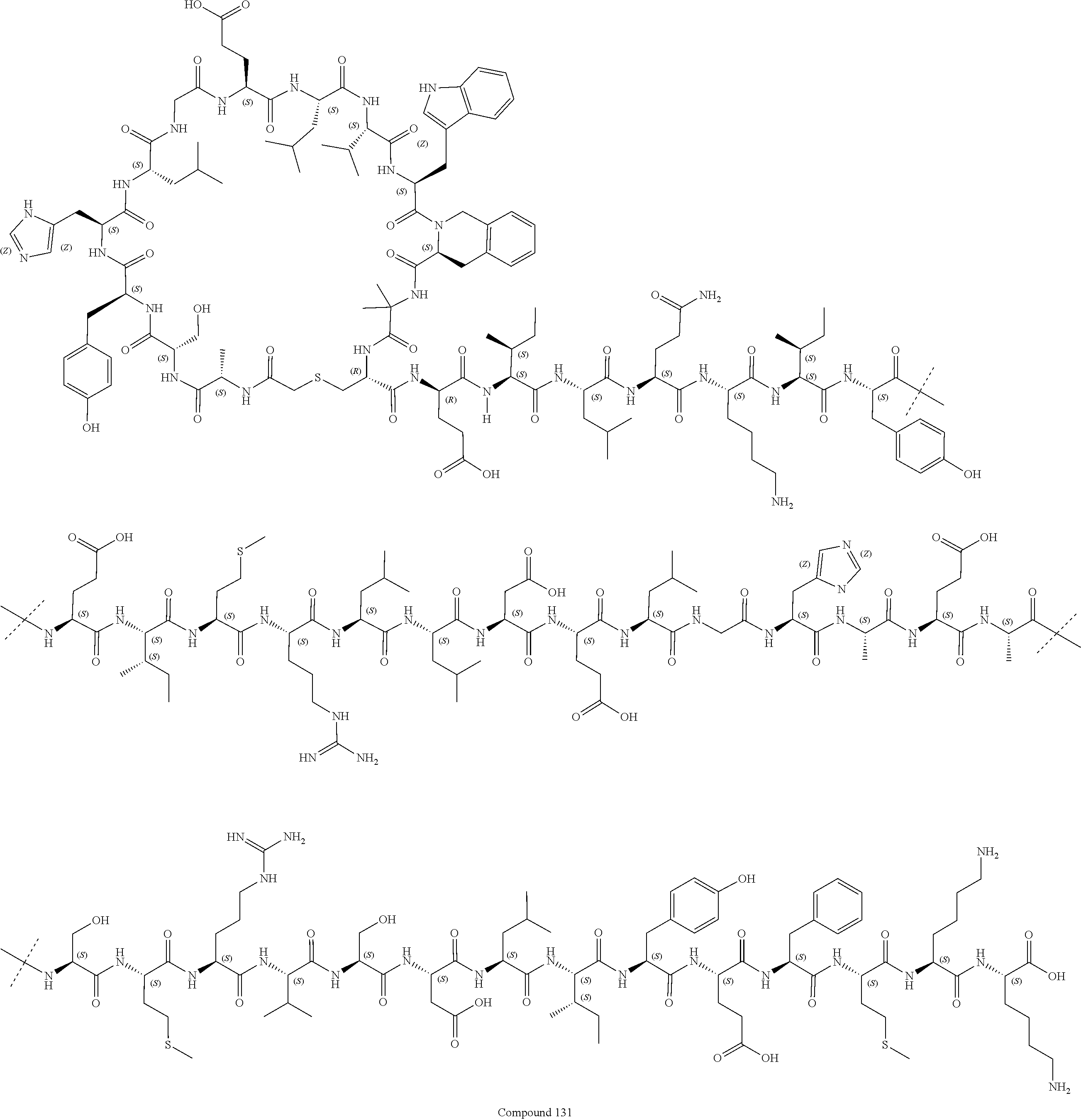

### Example 7. Alternate Procedure for Preparation of Compound 121 (Disulfide Cyclic Peptide)

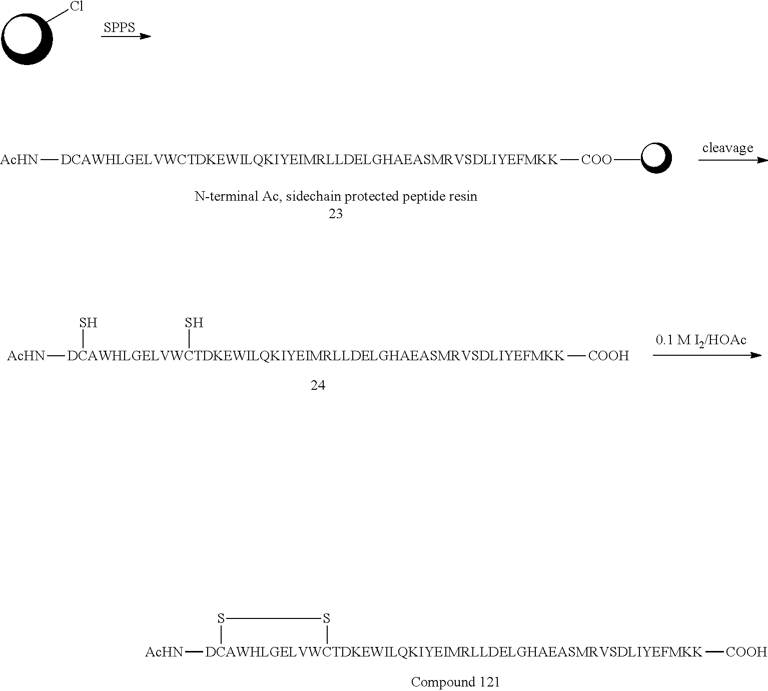

Resin-bound peptide (intermediate 23 (SEQ ID NO:21), CTC resin, 0.50 mmol) was synthesized using standard Fmoc chemistry.

### Peptide Cleavage, Cyclization and Purification:

Cleavage solution (TFA/TIS/H2O, 95/2.5/2.5, v/v/v, 20 mL) was added to the flask containing the side-chain protected resin-bound peptide (intermediate 23, CTC resin, 0.50 mmol) at room temperature and stirred for 2 h. The filtrate was collected.

The peptide was precipitated with cold isopropyl ether (100 mL) and centrifuged (3 min at 3000 rpm). The precipitate was washed with isopropyl ether (50 mL) twice, and the crude peptide was dried under reduced pressure for 2 h to afford intermediate 24 (SEQ ID NO:35)(4.30 g, crude) as a white solid.

To a mixture of intermediate 24 (4.30 g, crude) in AcOH/MeCN/H₂O (4/3/3, v/v/v, 500 mL) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (0.075% TFA/H₂O, MeCN), followed by lyophilization to afford Compound 121 (SEQ ID NO:22) (Batch1: 10.1 mg, 85.8% purity; batch2: 7.8 mg, 78.9% purity; 0.47% yield) as a white solid. LCMS: RT=2.24 min, MS cal.: 6198.15, mass observed: [M+4H]⁴⁺+=1550.40, [M+5H]⁵⁺=1240.80, [M+6H]⁶⁺=1033.90.

Disulfide cyclic peptides, Compounds 132, 133, 134, 135, 136, 137, and 138 were synthesized using the same method, which was performed by following the procedure for synthesis of intermediate 4.

0.50 mmol resin afforded Compound 132 (Batch1: 18.3 mg, 81.8% purity; batch2: 40.9 mg, 90.8% purity; 2.60% yield) as a white solid. LCMS: RT=1.77 min, MS cal.: 3889.50, [M+2H]²⁺=1945.20, [M+3H]³⁺=1297.2, [M+4H]⁴⁺=973.3.

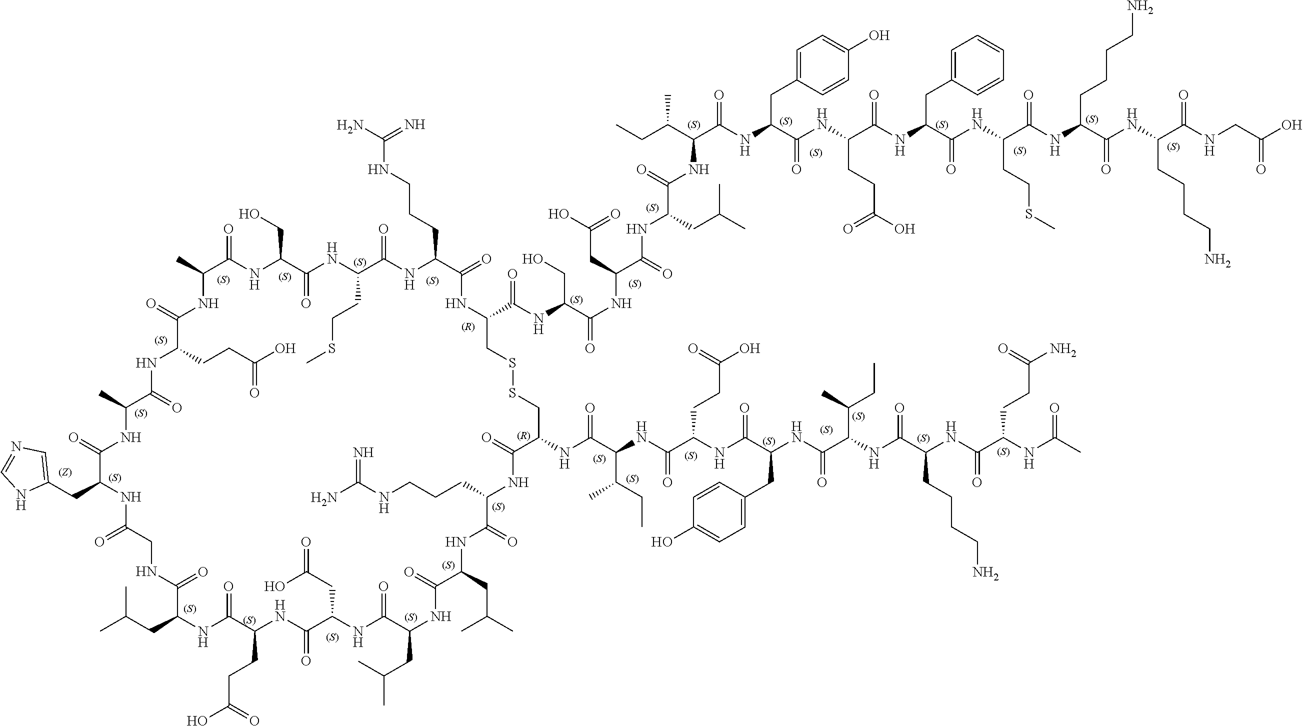

0.50 mmol resin afforded Compound 133 (Batch1: 18.7 mg, 93.2% purity; batch2: 37.5 mg, 96.0% purity; batch3: 58.3 mg, 96.9% purity; 5.47% yield) as a white solid. LCMS: RT=1.80 min, MS cal.: 3931.54, [M+2H]²⁺=1966.20, [M+3H]³⁺=1311.2, [M+4H]⁴⁺+=983.7.

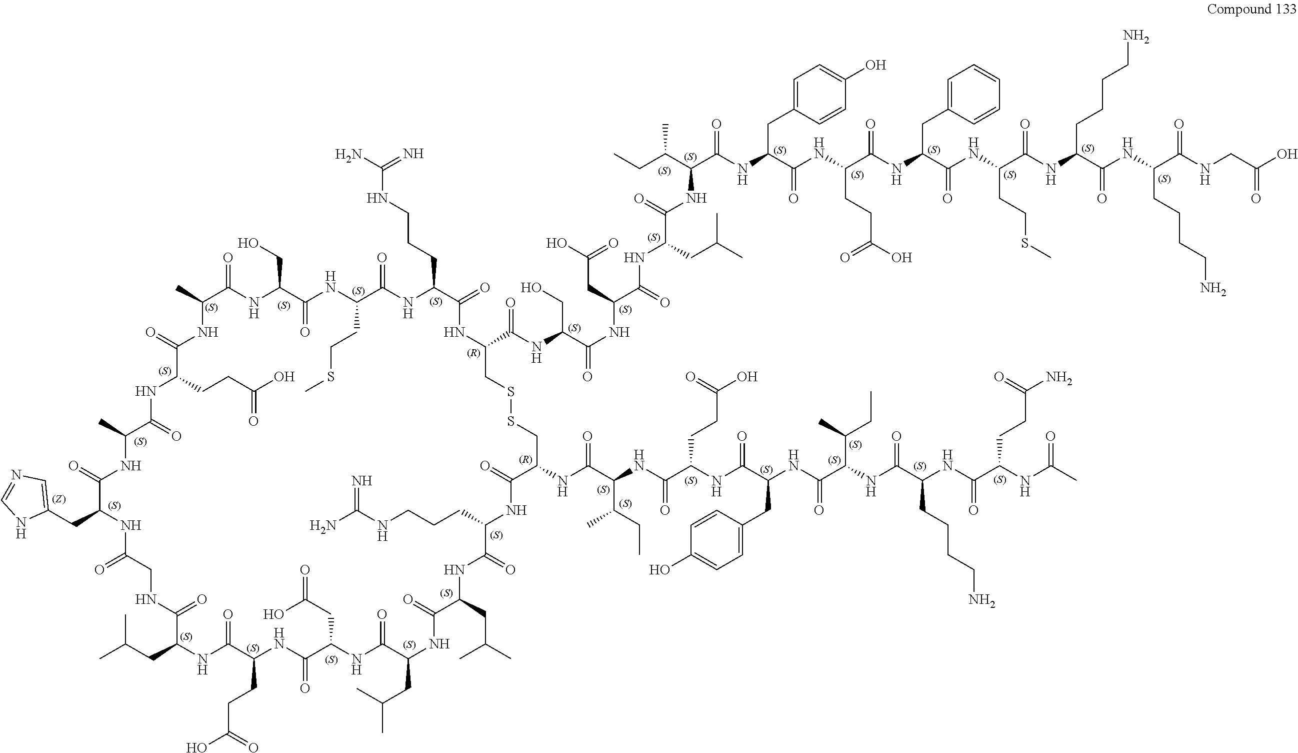

0.50 mmol resin afforded Compound 134 (batch 1: 29.2 mg, 91.9% purity; batch 2: 62.4 mg, 96.8% purity; 4.19% yield) as a white solid. LCMS: RT=1.80 min, MS cal.: 4115.82, [M+3H]³⁺=1372.70, [M+4H]⁴⁺+=1030.00.

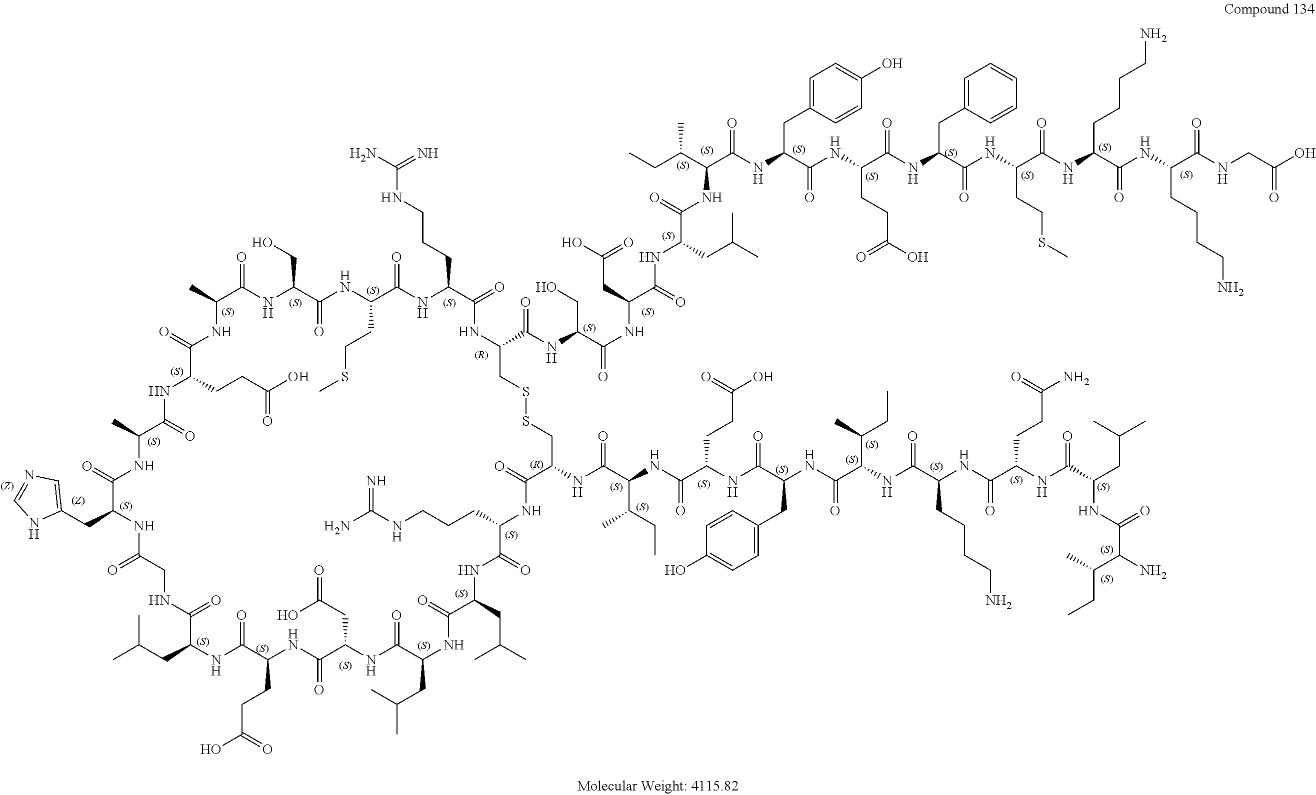

0.50 mmol resin afforded Compound 135 (batch 1: 26.6 mg, 91.2% purity; batch 2: 61.6 mg, 95.2% purity; 3.93% yield) as a white solid. LCMS: RT=1.92 min, MS cal.: 4157.85, [M+3H]³⁺=1386.80, [M+4H]⁴⁺=1040.20.

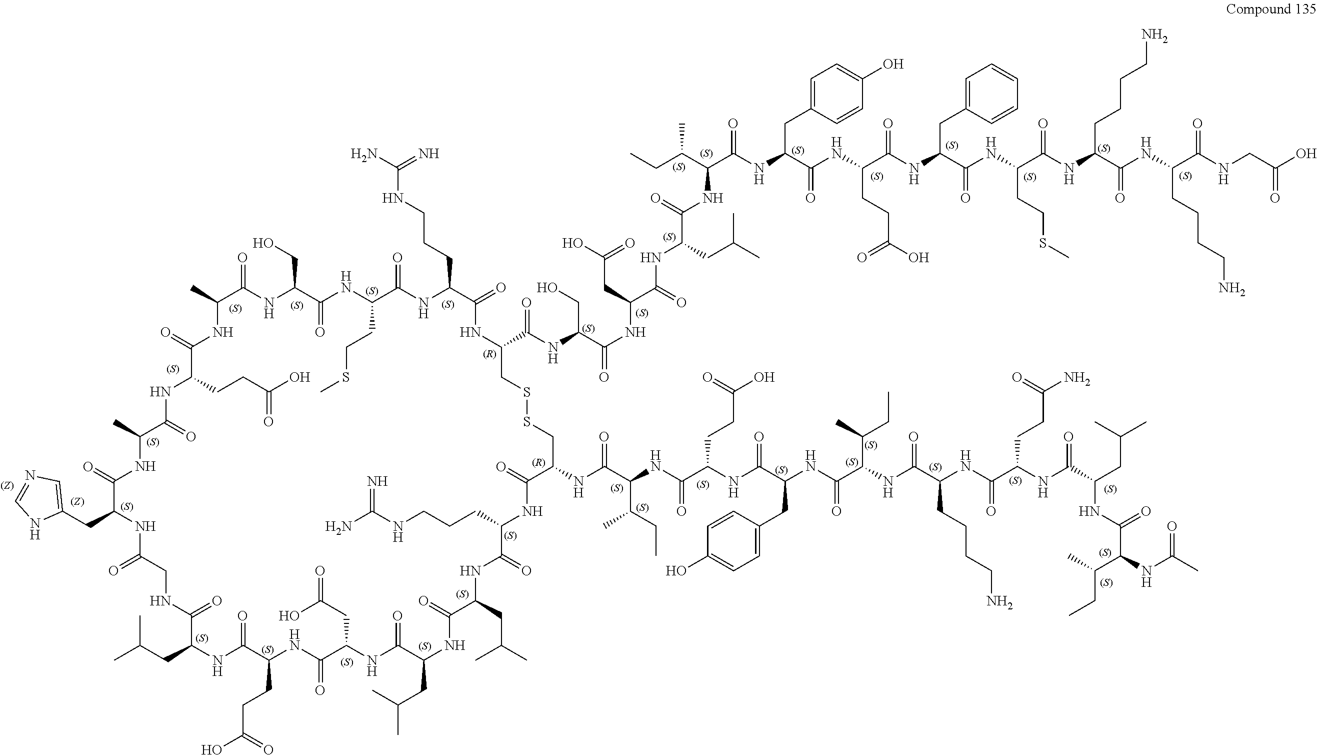

0.20 mmol resin afforded Compound 136 (Batch1: 11.8 mg, 85.4% purity; batch2: 5.9 mg, 95.3% purity; 1.6% yield in total) as a white solid. LCMS: RT=1.01 min, MS cal.: 4674.40, observed mass: [M+3H]³⁺=1559.06, [M+4H]⁴⁺+=1169.42.

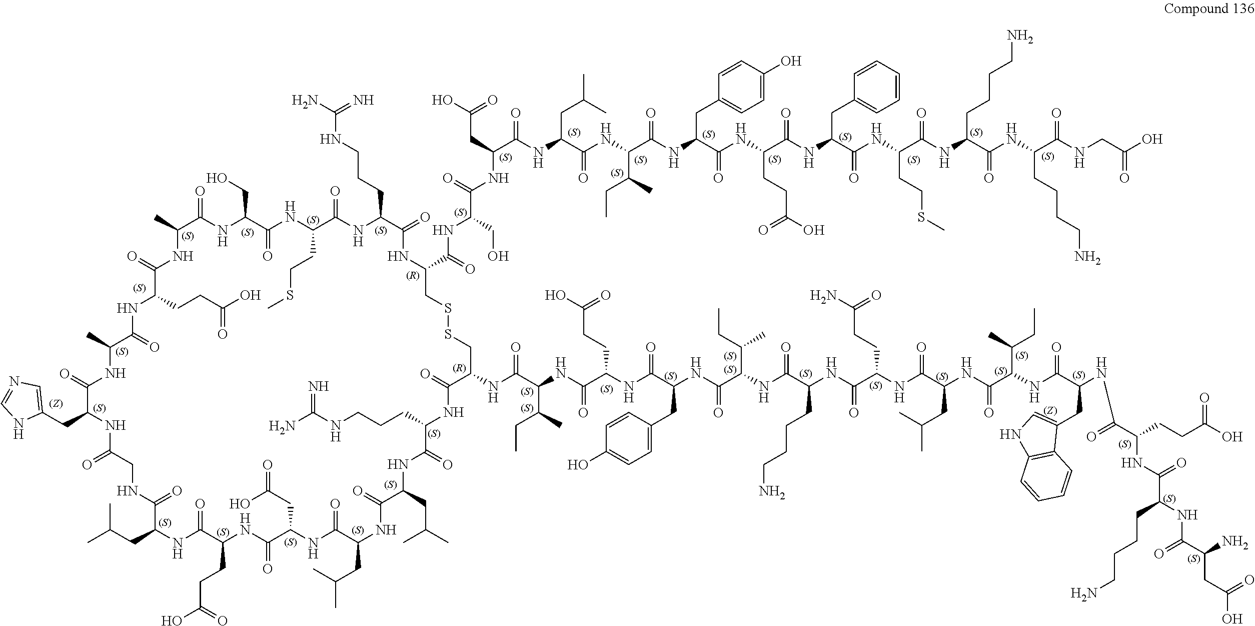

0.2 mmol resin afforded compound 137 (35.6 mg, 90.9% purity, 3.4% yield) as a white solid. TOF: RT=0.582 min, MS cal.: 4716.44, observed mass: [M+H]⁺=4716.2, [M+3H]³⁺=1572.77, [M+4H]⁴⁺=1180.08.

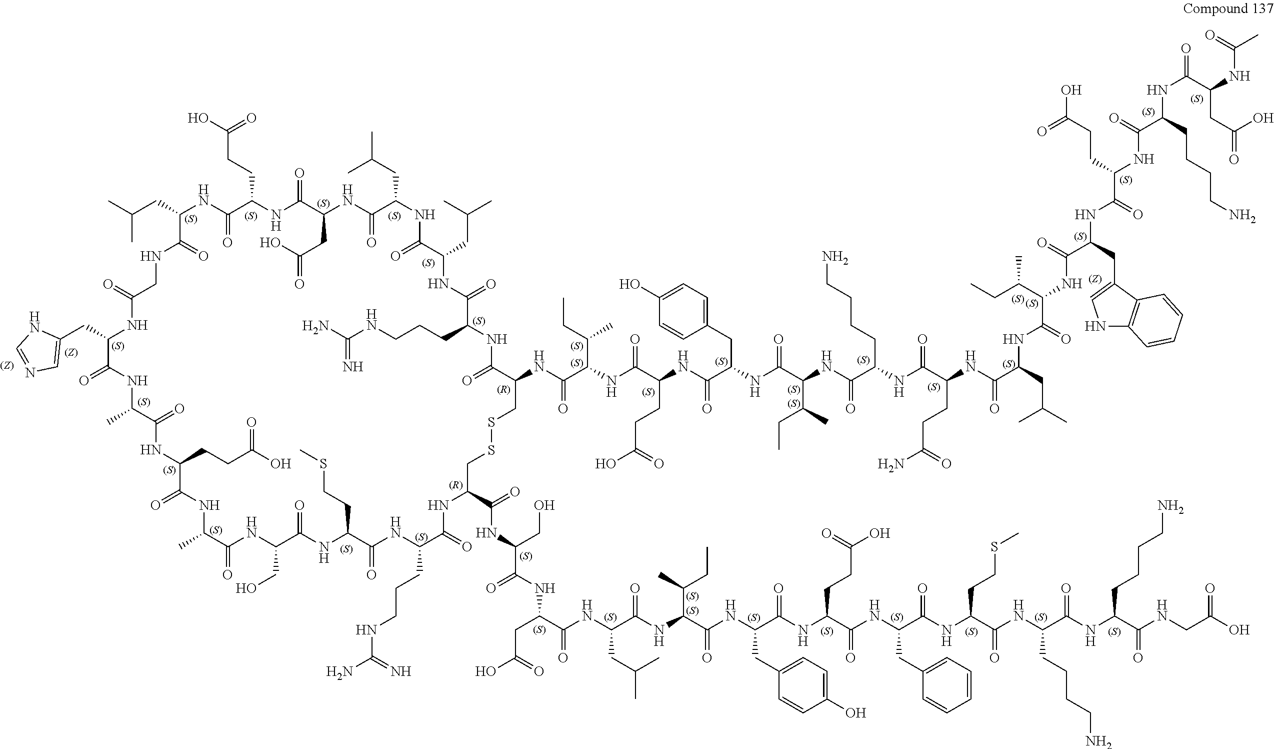

Disulfide cyclic peptide, Compound 138, was synthesized using the same method as intermediate 19. 0.20 mmol resin afforded Compound 138 (27.1 mg, 95.1% purity, 2.74% yield) as a white solid. LCMS: RT=1.04 min, MS cal.: 4703.51, observed mass: [M+3H]³⁺=1568.44, [M+4H]⁴⁺+=1176.63, [M+5H]⁵⁺=941.65.

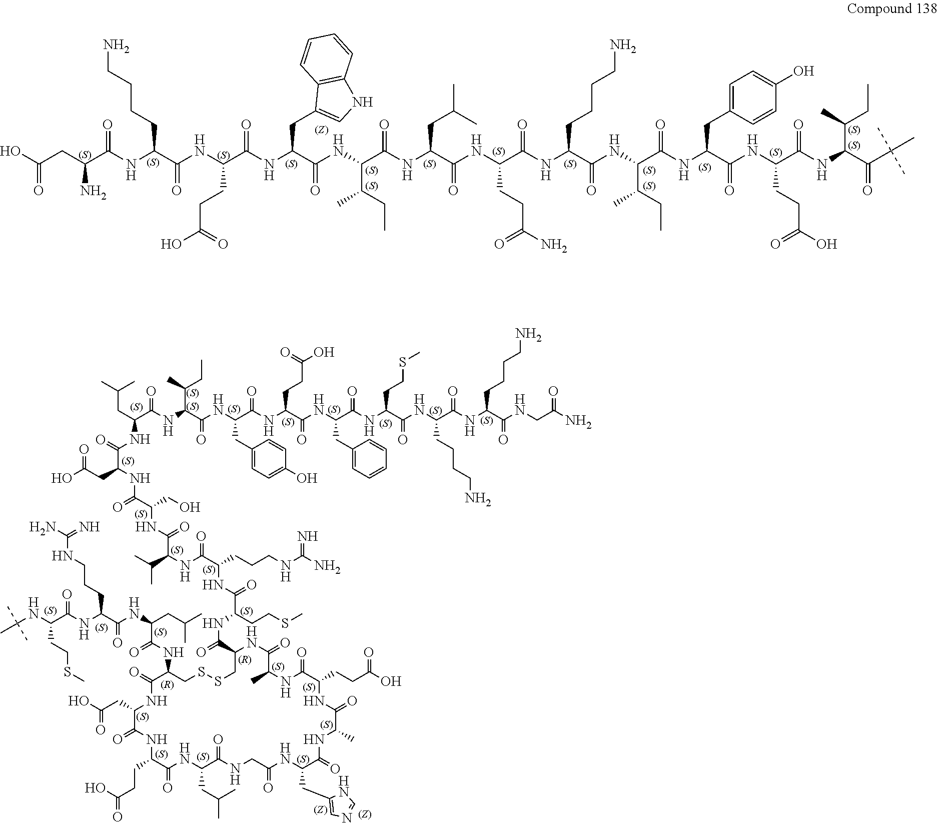

(SEQ ID NO: 23)

H2N-DKEWILQKIYEIMRLCDELGHAEACMRVSDLIYEFMKKG-NH2

### Example 8. Procedure for Preparation of Compound 139 (Bis-Disulfide Cyclic Peptide)

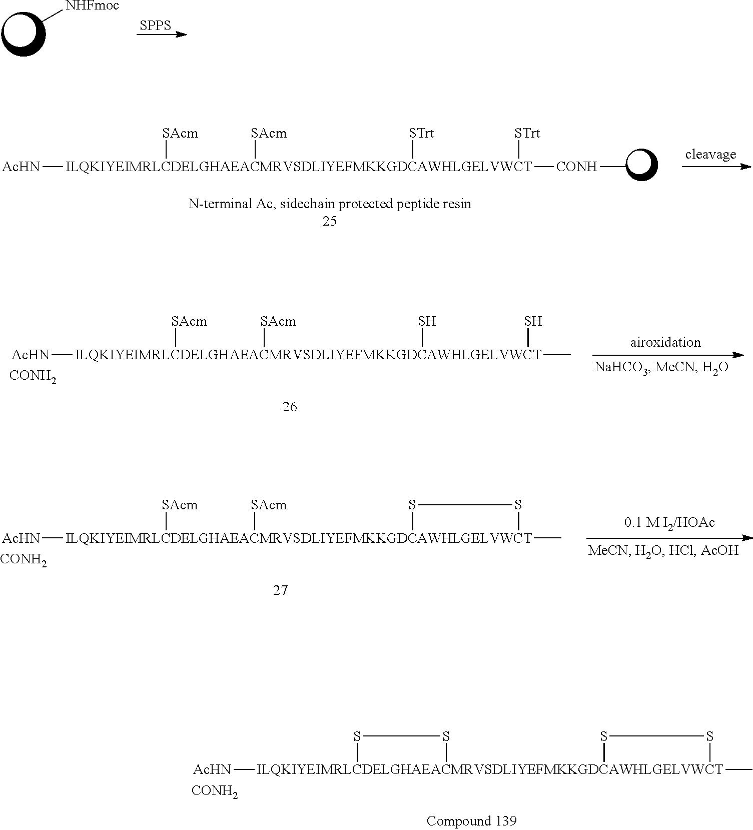

Peptide was synthesized using standard Fmoc chemistry (Rink amide resin).

Resin preparation: To the vessel containing Rink Amide AM resin (1.56 g, 0.50 mmol, 0.32 mmol/g) and DMF (20 mL) was bubbled with N₂ for 2 h at 20° C. Then 20% piperidine in DMF (40 mL) was added and the mixture was bubbled with N₂ for 30 min at 20° C. The mixture was filtered and washed with DMF (20 mL)*5 before proceeding to next step.
2) Coupling: A solution of Fmoc-Thr(tBu)-OH (0.60 g, 3.00 equiv.), HBTU (0.54 g, 2.85 equiv.) in DMF (20 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (40 mL)*5.
3) Deprotection: 20% piperidine in DMF (40 mL) was added to the resin and the mixture was bubbled with N₂ for 30 min at 20° C. The deprotection reaction was monitored by ninhydrin test. The resin was then washed with DMF (40 mL)*5.
4) The coupling and deprotection steps were repeated for the following amino acids elongation: Number #2-13, Table 6.
5) Coupling: A solution of Fmoc-Gly-OH (0.89 g, 6.00 equiv.), HBTU (1.09 g, 5.70 equiv.) in DMF (20 mL) was added to the resin with N₂ bubbling. Then DIEA (12.00 equiv.) was added to the mixture dropwise and bubbled with N₂ for 30 min at 20° C. The coupling reaction was monitored by ninhydrin test, if it showed colorless, the coupling was completed. The resin was then washed with DMF (40 mL)*5.
6) Deprotection: Step 3 was repeated for Fmoc deprotection.
7) Steps 5 and 6 were repeated for the following amino acids elongation: Number #15-48, Table 6.

Acetylation: A solution of Ac₂O/NMM/DMF (2/1/17, v/v/v, 20 mL) was added to resin, the mixture was bubbled with N₂ for 20 min. The acetylation reaction was monitored by ninhydrin test. The resin was then washed with DMF (20 mL)*5, MeOH (20 mL)*3, then dried under reduced pressure to afford intermediate 25 (SEQ ID NO:24) (Rink AM resin, 0.5 mmol, 4.50 g).

TABLE 6
The list of amino acids and the corresponding reagents used on SPPS.

| # | Materials | Coupling reagents |
|---|---|---|
| 1 | Fmoc-Thr(tBu)-OH (3.00 equiv.) | HBTU (2.85 equiv.) and DIEA (6.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-His(Trt)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |
| 14 | Fmoc-Gly-OH (6.00 equiv.) | HATU (5.70 equiv.) and DIEA (12.00 equiv.) |
| 15 | Fmoc-Lys(Boc)-OH (6.00 equiv.) | HATU (5.70 equiv.) and DIEA (12.00 equiv.) |
| 16 | Fmoc-Lys(Boc)-OH (6.00 equiv.) | HATU (5.70 equiv.) and DIEA (12.00 equiv.) |
| 17 | Fmoc-Met-OH (6.00 equiv.) | HATU (5.70 equiv.) and DIEA (12.00 equiv.) |
| 18 | Fmoc-Phe-OH (6.00 equiv.) | HATU (5.70 equiv.) and DIEA (12.00 equiv.) |
| 19 | Fmoc-Glu(OtBu)-OH (6.00 equiv.) | HATU (5.70 equiv.) and DIEA (12.00 equiv.) |
| 20 | Fmoc-Tyr(tBu)-OH (6.00 equiv.) | HATU (5.70 equiv.) and DIEA (12.00 equiv.) |
| 21 | Fmoc-Ile-OH (6.00 equiv.) | HATU (5.70 equiv.) and DIEA (12.00 equiv.) |
| 22 | Fmoc-Leu-OH (6.00 equiv.) | HATU (5.70 equiv.) and DIEA (12.00 equiv.) |
| 23 | Fmoc-Asp(OtBu)-OH (6.00 equiv.) | HATU (5.70 equiv.) and DIEA (12.00 equiv.) |
| 24 | Fmoc-Ser(tBu)-OH (6.00 equiv.) | HATU (5.70 equiv.) and DIEA (12.00 equiv.) |
| 25 | Fmoc-Val-OH (6.00 equiv.) | HATU (5.70 equiv.) and DIEA (12.00 equiv.) |
| 26 | Fmoc-Arg(Pbf)-OH (6.00 equiv.) | HATU (5.70 equiv.) and DIEA (12.00 equiv.) |
| 27 | Fmoc-Met-OH (6.00 equiv.) | HATU (5.70 equiv.) and DIEA (12.00 equiv.) |
| 28 | Fmoc-Cys(Acm)-OH (6.00 equiv.) | HATU (5.70 equiv.) and DIEA (12.00 equiv.) |
| 29 | Fmoc-Ala-OH (6.00 equiv.) | HATU (5.70 equiv.) and DIEA (12.00 equiv.) |
| 30 | Fmoc-Glu(OtBu)-OH (6.00 equiv.) | HATU (5.70 equiv.) and DIEA (12.00 equiv.) |
| 31 | Fmoc-Ala-OH (6.00 equiv.) | HATU (5.70 equiv.) and DIEA (12.00 equiv.) |
| 32 | Fmoc-His(Trt)-OH (6.00 equiv.) | HATU (5.70 equiv.) and DIEA (12.00 equiv.) |
| 33 | Fmoc-Gly-OH (6.00 equiv.) | HATU (5.70 equiv.) and DIEA (12.00 equiv.) |
| 34 | Fmoc-Leu-OH (6.00 equiv.) | HATU (5.70 equiv.) and DIEA (12.00 equiv.) |
| 35 | Fmoc-Glu(OtBu)-OH (6.00 equiv.) | HATU (5.70 equiv.) and DIEA (12.00 equiv.) |
| 36 | Fmoc-Asp(OtBu)-OH (6.00 equiv.) | HATU (5.70 equiv.) and DIEA (12.00 equiv.) |
| 37 | Fmoc-Cys(Acm)-OH (6.00 equiv.) | HATU (5.70 equiv.) and DIEA (12.00 equiv.) |
| 38 | Fmoc-Leu-OH (6.00 equiv.) | HATU (5.70 equiv.) and DIEA (12.00 equiv.) |
| 39 | Fmoc-Arg(Pbf)-OH (6.00 equiv.) | HATU (5.70 equiv.) and DIEA (12.00 equiv.) |
| 40 | Fmoc-Met-OH (6.00 equiv.) | HATU (5.70 equiv.) and DIEA (12.00 equiv.) |
| 41 | Fmoc-Ile-OH (6.00 equiv.) | HATU (5.70 equiv.) and DIEA (12.00 equiv.) |
| 42 | Fmoc-Glu(OtBu)-OH (6.00 equiv.) | HATU (5.70 equiv.) and DIEA (12.00 equiv.) |
| 43 | Fmoc-Tyr(tBu)-OH (6.00 equiv.) | HATU (5.70 equiv.) and DIEA (12.00 equiv.) |
| 44 | Fmoc-Ile-OH (6.00 equiv.) | HATU (5.70 equiv.) and DIEA (12.00 equiv.) |
| 45 | Fmoc-Lys(Boc)-OH (6.00 equiv.) | HATU (5.70 equiv.) and DIEA (12.00 equiv.) |
| 46 | Fmoc-Gln(Trt)-OH (6.00 equiv.) | HATU (5.70 equiv.) and DIEA (12.00 equiv.) |
| 47 | Fmoc-Leu-OH (6.00 equiv.) | HATU (5.70 equiv.) and DIEA (12.00 equiv.) |
| 48 | Fmoc-Ile-OH (6.00 equiv.) | HATU (5.70 equiv.) and DIEA (12.00 equiv.) |
| 49 | Acetylation | NMM/Ac2O/DMF (1/2/17, 20 mL) |

Peptide Cleavage, cyclization and purification: Cleavage solution (TFA/TIS/H₂O, 95/2.5/2.5, v/v/v, 80 mL) was added to the flask containing the side-chain protected resin-bound peptide intermediate 25 (SEQ ID NO:24) (Rink AM resin, 0.5 mmol, 4.50 g) at room temperature and stirred for 2h.

After filtration, the filtrate was collected. The filtrate was precipitated with cold isopropyl ether (800 mL). After filtration, the solid was washed with isopropyl ether (200 mL) twice, and the crude peptide was dried under reduced pressure for 2 h to afford intermediate 26 (SEQ ID NO:25) (3.10 g, crude) as a white solid.

To a mixture of intermediate 26 (3.10 g, crude) in MeCN/H₂O (4/6, v/v, 500 mL) was added NaHCO₃ to pH=8 at 20° C. Then the mixture was open to the air and stirred at 20° C. for 24 h. after filtration, the mixture was purified by prep-HPLC (A: 0.075% TFA/H₂O, B: MeCN) to afford intermediate 27 (SEQ ID NO:26) (92.8 mg, 90.0% purity, 3.26% yield) as a white solid.

To a mixture of intermediate 27 (92.8 mg, 91.5% purity) in MeCN/H₂O (4/6, v/v, 50 mL) was added 1 M aq. HCl (1.5 mL), AcOH (3.0 mL) to pH=1. The mixture was added 1 M I₂/AcOH dropwise until the yellow color persisted, then the mixture was stirred at 20° C. for 1 h. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford Compound 139 (SEQ ID NO:27) (Batch1: 10.2 mg, 93.0% purity; batch2: 20.5 mg, 89.3% purity; 33.1% yield) as a white solid. LCMS: RT=2.19 min, MS cal.: 5700.66, mass observed: [M+3H]³⁺=1900.10, [M+4H]⁴⁺+=1425.80, [M+5H]⁵⁺=1140.60.

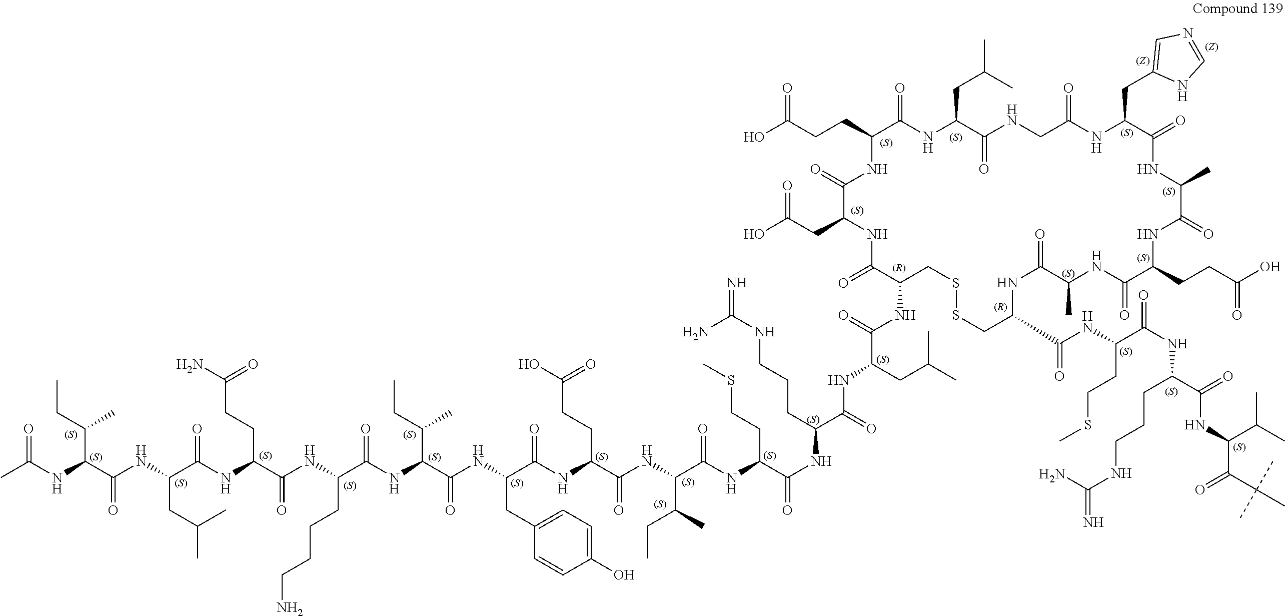

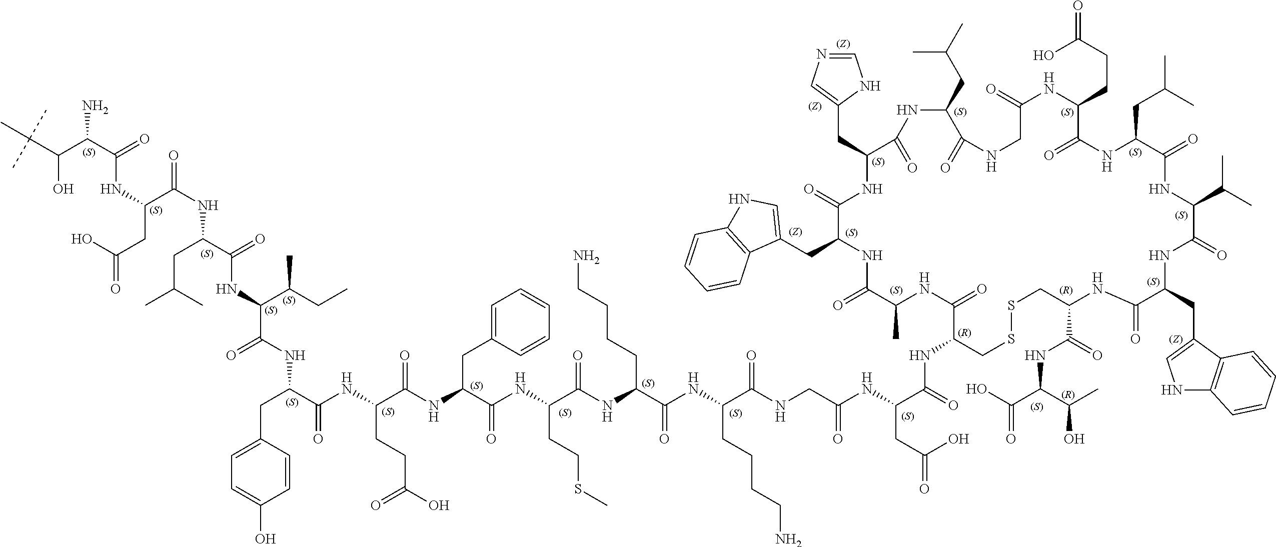

(SEQ ID NO: 36)

Ace-ILQKIYEIMRLCDELGHAEACMRVSDLIYEFMKKG-uABT-NH2

Compounds 140 and 141 were synthesized using the same method as Compound 139 (SEQ ID NO:27).

0.50 mmol resin afforded Compound 140 (Batch1: 5.4 mg, 87.6% purity; batch2: 16.6 mg, 94.1% purity; 0.73% yield) as a white solid. LCMS: RT=1.93 min, MS cal.: 5443.26, observed mass: [M+3H]³⁺=1814.60, [M+4H]⁴⁺=1361.60, [M+5H]⁵⁺=1089.2.

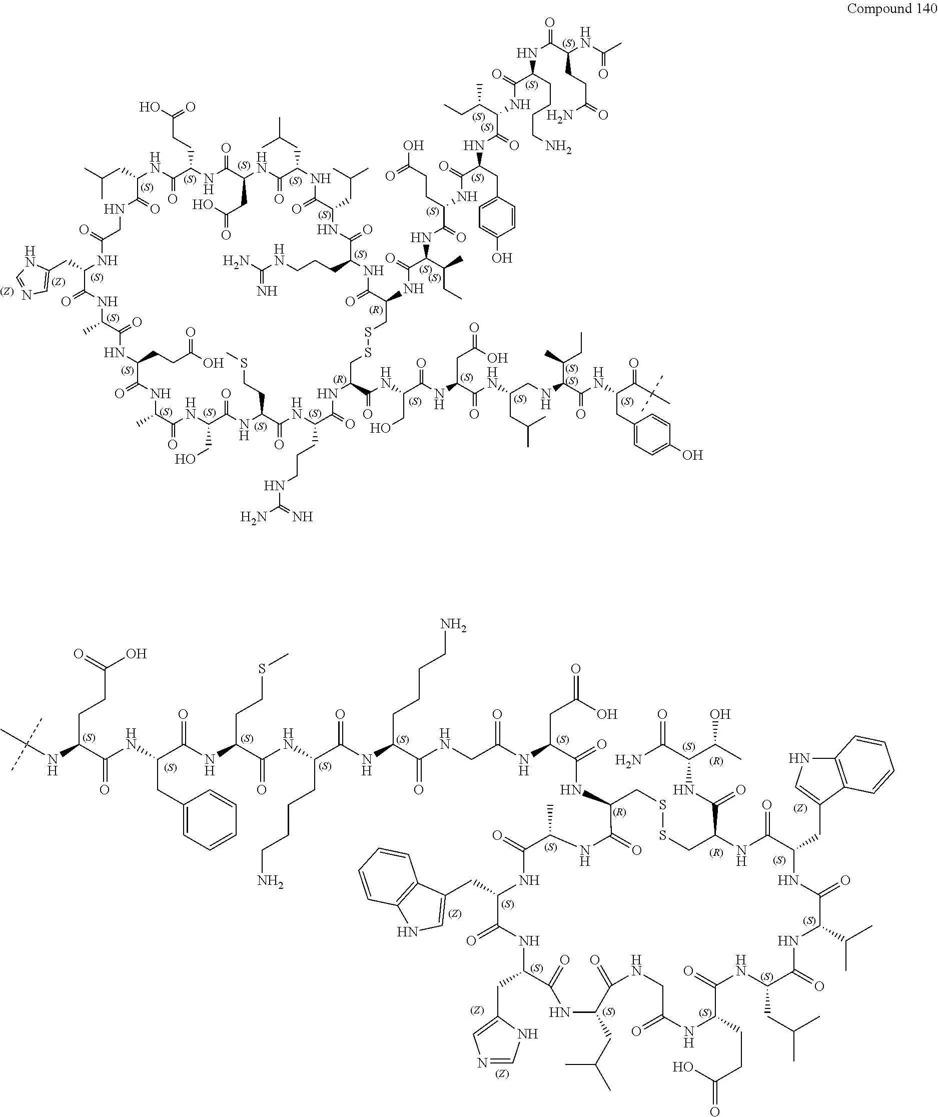

0.5 mmol resin afforded Compound 141 (16.70 mg, 97.6% purity, 1.08% yield) as a white solid. LCMS: RT=2.01 min, MS cal.: 5669.58, observed mass: [M+3H]³⁺=1890.20, [M+4H]⁴⁺+=1418.10, [M+5H]⁵⁺=1134.4.

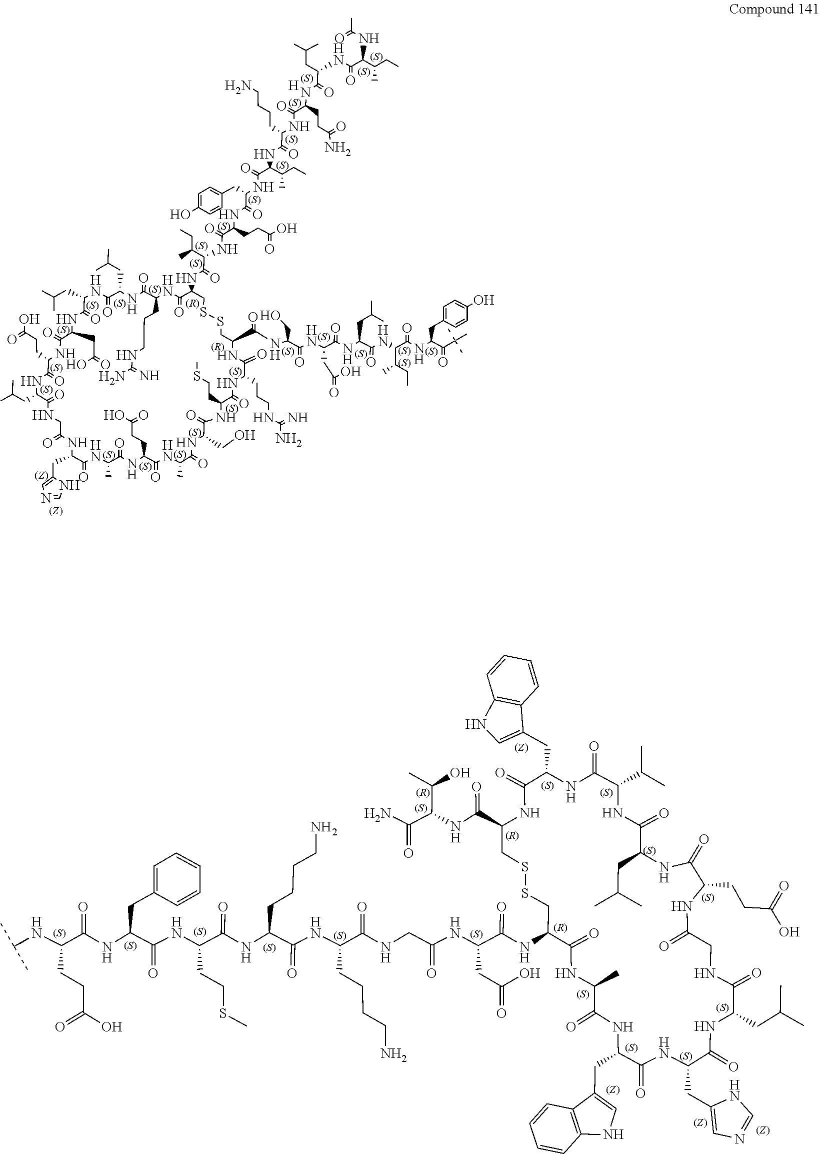

### Example 9. Procedure for Preparation of Compound 142 (uABT Disulfide Cyclic Peptide)

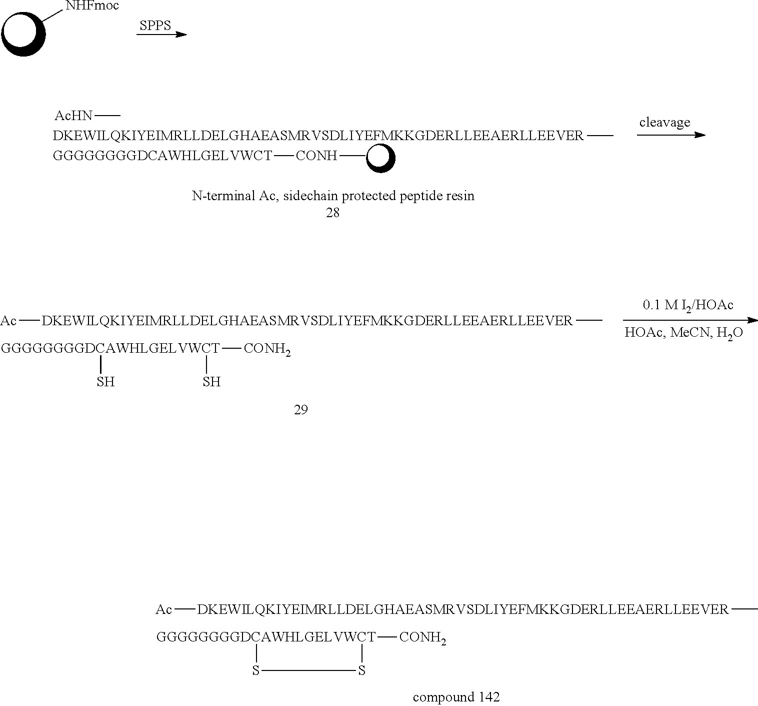

Peptide was synthesized using standard Fmoc chemistry (Rink amide resin).

Resin preparation: To the vessel containing Rink Amide AM resin (1.56 g, 0.50 mmol, 0.32 mmol/g) and DMF (20 mL) was bubbled with N₂ for 2 h at 20° C. Then 20% piperidine in DMF (40 mL) was added and the mixture was bubbled with N₂ for 30 min at 20° C. The mixture was filtered and washed with DMF (20 mL)*5 before proceeding to next step.

Coupling: A solution of Fmoc-Thr(tBu)-OH (595.5 mg, 1.50 mmol, 3.00 equiv.), DIC (189.0 mg, 5.70 mmol, 3.00 equiv.), HOBt (202.5 mg, 1.50 mmol, 3.00 equiv.) in DMF (20 mL) was added to the resin with N₂ bubbling. The mixture was bubbled with N₂ for 1 h at 20° C. The coupling reaction was monitored by ninhydrin test. The resin was then washed with DMF (40 mL)*5.

Deprotection: 20% piperidine in DMF (40 mL) was added to the resin and the mixture was bubbled with N₂ for 30 min at 20° C. The deprotection reaction was monitored by ninhydrin test. The resin was then washed with DMF (40 mL)*5.

The coupling and deprotection steps were repeated for the following amino acids elongation: Number #2-72, Table 7.

Acetylation: A solution of Ac₂O/NMM/DMF (2/1/17, v/v/v, 20 mL) was added to resin, the mixture was bubbled with N₂ for 20 min. The acetylation reaction was monitored by ninhydrin test. The resin was then washed with DMF (20 mL)*5, MeOH (20 mL)*3, then dried under reduced pressure to afford intermediate 28 (SEQ ID NO:28) (Rink AM resin, 0.5 mmol, 5.30 g).

TABLE 7
The list of amino acids and the corresponding reagents used on SPPS.

| # | Materials | Coupling reagents |
|---|---|---|
| 1 | Fmoc-Thr(tBu)-OH (3.00 equiv.) | DIC (3.00 equiv.) and HOBt (3.00 equiv.) |
| 2 | Fmoc-Cys(Trt)-OH (3.00 equiv.) | DIC (3.00 equiv.) and HOBt (3.00 equiv.) |
| 3 | Fmoc-Trp-OH (3.00 equiv.) | DIC (3.00 equiv.) and HOBt (3.00 equiv.) |
| 4 | Fmoc-Val-OH (3.00 equiv.) | DIC (3.00 equiv.) and HOBt (3.00 equiv.) |
| 5 | Fmoc-Leu-OH (3.00 equiv.) | DIC (3.00 equiv.) and HOBt (3.00 equiv.) |
| 6 | Fmoc-Glu(OtBu)-OH (3.00 equiv.) | DIC (3.00 equiv.) and HOBt (3.00 equiv.) |
| 7 | Fmoc-Gly-OH (3.00 equiv.) | DIC (3.00 equiv.) and HOBt (3.00 equiv.) |
| 8 | Fmoc-Leu-OH (3.00 equiv.) | DIC (3.00 equiv.) and HOBt (3.00 equiv.) |
| 9 | Fmoc-His(Trt)-OH (3.00 equiv.) | DIC (3.00 equiv.) and HOBt (3.00 equiv.) |
| 10 | Fmoc-Trp-OH (3.00 equiv.) | DIC (3.00 equiv.) and HOBt (3.00 equiv.) |
| 11 | Fmoc-Ala-OH (3.00 equiv.) | DIC (3.00 equiv.) and HOBt (3.00 equiv.) |
| 12 | Fmoc-Cys(Trt)-OH (3.00 equiv.) | DIC (3.00 equiv.) and HOBt (3.00 equiv.) |
| 13 | Fmoc-Asp(OtBu)-OH (3.00 equiv.) | DIC (3.00 equiv.) and HOBt (3.00 equiv.) |
| 14 | Fmoc-Gly-Gly-OH (6.00 equiv.) | DIC (6.00 equiv.) and HOBt (6.00 equiv.) |
| 15 | Fmoc-Gly-Gly-Gly-OH (6.00 equiv.) | DIC (6.00 equiv.) and HOBt (6.00 equiv.) |
| 16 | Fmoc-Gly-Gly-Gly-OH (6.00 equiv.) | DIC (6.00 equiv.) and HOBt (6.00 equiv.) |
| 17 | Fmoc-Arg(Pbf)-OH (6.00 equiv.) | DIC (6.00 equiv.) and HOBt (6.00 equiv.) |
| 18 | Fmoc-Glu(OtBu)-OH (6.00 equiv.) | DIC (6.00 equiv.) and HOBt (6.00 equiv.) |
| 19 | Fmoc-Val-OH (6.00 equiv.) | DIC (6.00 equiv.) and HOBt (6.00 equiv.) |
| 20 | Fmoc-Glu(OtBu)-OH (6.00 equiv.) | DIC (6.00 equiv.) and HOBt (6.00 equiv.) |
| 21 | Fmoc-Glu(OtBu)-OH (6.00 equiv.) | DIC (6.00 equiv.) and HOBt (6.00 equiv.) |
| 22 | Fmoc-Leu-OH (6.00 equiv.) | DIC (6.00 equiv.) and HOBt (6.00 equiv.) |
| 23 | Fmoc-Leu-OH (6.00 equiv.) | DIC (6.00 equiv.) and HOBt (6.00 equiv.) |
| 24 | Fmoc-Arg(Pbf)-OH (6.00 equiv.) | DIC (6.00 equiv.) and HOBt (6.00 equiv.) |
| 25 | Fmoc-Glu(OtBu)-OH (6.00 equiv.) | DIC (6.00 equiv.) and HOBt (6.00 equiv.) |
| 26 | Fmoc-Ala-OH (6.00 equiv.) | DIC (6.00 equiv.) and HOBt (6.00 equiv.) |
| 27 | Fmoc-Glu(OtBu)-OH (6.00 equiv.) | DIC (6.00 equiv.) and HOBt (6.00 equiv.) |
| 28 | Fmoc-Glu(OtBu)-OH (6.00 equiv.) | DIC (6.00 equiv.) and HOBt (6.00 equiv.) |
| 29 | Fmoc-Leu-OH (6.00 equiv.) | DIC (6.00 equiv.) and HOBt (6.00 equiv.) |
| 30 | Fmoc-Leu-OH (6.00 equiv.) | DIC (6.00 equiv.) and HOBt (6.00 equiv.) |
| 31 | Fmoc-Arg(Pbf)-OH (6.00 equiv.) | DIC (6.00 equiv.) and HOBt (6.00 equiv.) |
| 32 | Fmoc-Glu(OtBu)-OH (6.00 equiv.) | DIC (6.00 equiv.) and HOBt (6.00 equiv.) |
| 33 | Fmoc-Asp(OtBu)-OH (3.00 equiv.) | DIC (6.00 equiv.) and HOBt (6.00 equiv.) |
| 34 | Fmoc-Gly-OH (6.00 equiv.) | DIC (6.00 equiv.) and HOBt (6.00 equiv.) |
| 35 | Fmoc-Lys(Boc)-OH (6.00 equiv.) | DIC (6.00 equiv.) and HOBt (6.00 equiv.) |
| 36 | Fmoc-Lys(Boc)-OH (6.00 equiv.) | DIC (6.00 equiv.) and HOBt (6.00 equiv.) |
| 37 | Fmoc-Met-OH (6.00 equiv.) | DIC (6.00 equiv.) and HOBt (6.00 equiv.) |
| 38 | Fmoc-Phe-OH (6.00 equiv.) | DIC (6.00 equiv.) and HOBt (6.00 equiv.) |
| 39 | Fmoc-Glu(OtBu)-OH (6.00 equiv.) | DIC (6.00 equiv.) and HOBt (6.00 equiv.) |
| 40 | Fmoc-Tyr(tBu)-OH (9.00 equiv.) | DIC (9.00 equiv.) and HOBt (9.00 equiv.) |
| 41 | Fmoc-Ile-OH (9.00 equiv.) | DIC (9.00 equiv.) and HOBt (9.00 equiv.) |
| 42 | Fmoc-Leu-OH (9.00 equiv.) | DIC (9.00 equiv.) and HOBt (9.00 equiv.) |
| 43 | Fmoc-Asp(OtBu)-OH (9.00 equiv.) | DIC (9.00 equiv.) and HOBt (9.00 equiv.) |
| 44 | Fmoc-Ser(tBu)-OH (9.00 equiv.) | DIC (9.00 equiv.) and HOBt (9.00 equiv.) |
| 45 | Fmoc-Val-OH (9.00 equiv.) | DIC (9.00 equiv.) and HOBt (9.00 equiv.) |
| 46 | Fmoc-Arg(Pbf)-OH (9.00 equiv.) | DIC (9.00 equiv.) and HOBt (9.00 equiv.) |
| 47 | Fmoc-Met-OH (9.00 equiv.) | DIC (9.00 equiv.) and HOBt (9.00 equiv.) |
| 48 | Fmoc-Ser(tBu)-OH (9.00 equiv.) | DIC (9.00 equiv.) and HOBt (9.00 equiv.) |
| 49 | Fmoc-Ala-OH (9.00 equiv.) | DIC (9.00 equiv.) and HOBt (9.00 equiv.) |
| 50 | Fmoc-Glu(OtBu)-OH (9.00 equiv.) | DIC (9.00 equiv.) and HOBt (9.00 equiv.) |
| 51 | Fmoc-Ala-OH (9.00 equiv.) | DIC (9.00 equiv.) and HOBt (9.00 equiv.) |
| 52 | Fmoc-His(Trt)-OH (9.00 equiv.) | DIC (9.00 equiv.) and HOBt (9.00 equiv.) |
| 53 | Fmoc-Gly-OH (9.00 equiv.) | DIC (9.00 equiv.) and HOBt (9.00 equiv.) |
| 54 | Fmoc-Leu-OH (9.00 equiv.) | DIC (9.00 equiv.) and HOBt (9.00 equiv.) |
| 55 | Fmoc-Glu(OtBu)-OH (9.00 equiv.) | DIC (9.00 equiv.) and HOBt (9.00 equiv.) |
| 56 | Fmoc-Asp(OtBu)-OH (9.00 equiv.) | DIC (9.00 equiv.) and HOBt (9.00 equiv.) |
| 57 | Fmoc-Leu-OH (9.00 equiv.) | DIC (9.00 equiv.) and HOBt (9.00 equiv.) |
| 58 | Fmoc-Leu-OH (9.00 equiv.) | DIC (9.00 equiv.) and HOBt (9.00 equiv.) |
| 59 | Fmoc-Arg(Pbf)-OH (9.00 equiv.) | DIC (9.00 equiv.) and HOBt (9.00 equiv.) |
| 60 | Fmoc-Met-OH (9.00 equiv.) | DIC (9.00 equiv.) and HOBt (9.00 equiv.) |
| 61 | Fmoc-Ile-OH (9.00 equiv.) | DIC (9.00 equiv.) and HOBt (9.00 equiv.) |
| 62 | Fmoc-Glu(OtBu)-OH (9.00 equiv.) | DIC (9.00 equiv.) and HOBt (9.00 equiv.) |
| 63 | Fmoc-Tyr(tBu)-OH (9.00 equiv.) | DIC (9.00 equiv.) and HOBt (9.00 equiv.) |
| 64 | Fmoc-Ile-OH (9.00 equiv.) | DIC (9.00 equiv.) and HOBt (9.00 equiv.) |
| 65 | Fmoc-Lys(Boc)-OH (9.00 equiv.) | DIC (9.00 equiv.) and HOBt (9.00 equiv.) |
| 66 | Fmoc-Gln(Trt)-OH (9.00 equiv.) | DIC (9.00 equiv.) and HOBt (9.00 equiv.) |
| 67 | Fmoc-Leu-OH (9.00 equiv.) | DIC (9.00 equiv.) and HOBt (9.00 equiv.) |
| 68 | Fmoc-Ile-OH (9.00 equiv.) | DIC (9.00 equiv.) and HOBt (9.00 equiv.) |
| 69 | Fmoc-Trp-OH (9.00 equiv.) | DIC (9.00 equiv.) and HOBt (9.00 equiv.) |
| 70 | Fmoc-Glu(OtBu)-OH (9.00 equiv.) | DIC (9.00 equiv.) and HOBt (9.00 equiv.) |
| 71 | Fmoc-Lys(Boc)-OH (9.00 equiv.) | DIC (9.00 equiv.) and HOBt (9.00 equiv.) |
| 72 | Fmoc-Asp(OtBu)-OH (9.00 equiv.) | DIC (9.00 equiv.) and HOBt (9.00 equiv.) |
| 73 | Acetylation | NMM/Ac20/DMF (1/2/17, v/v/v, 20 mL) |

Peptide cleavage, cyclization and purification: Cleavage solution (TFA/TIS/H₂O, 95/2.5/2.5, v/v/v, 100 mL) was added to the flask containing the side-chain protected resin-bound peptide intermediate 28 (SEQ ID NO:28) (Rink AM resin, 0.5 mmol, 5.30 g) at room temperature and stirred for 2h.

After filtration, the filtrate was collected. The filtrate was precipitated with cold isopropyl ether (1.0 L). After filtration, the solid was washed with isopropyl ether (200 mL) twice, and the crude peptide was dried under reduced pressure for 2 h to afford intermediate 17 (3.82 g, crude) as a white solid.

To a mixture of intermediate 29 (SEQ ID NO:29) (3.82 g, crude) in HOAc/MeCN/H₂O (4/3/3, v/v/v, 2.0 L) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (0.075% TFA/H₂O, MeCN), followed by lyophilization to afford Compound 142 (SEQ ID NO:30) (batch 1: 3.0 mg, 88.6% purity; batch2: 6.6 mg, 83.7% purity; 0.22% yield) as a white solid. LCMS: RT=2.29 min, MS cal.: 8820.91, mass observed: [M+5H]⁵⁺=1765.50, [M+6H]⁶⁺=1471.0 [M+7H]⁷⁺=1261.30, [M+8H]⁸⁺=1103.70, [M+9H]⁹⁺=981.30.

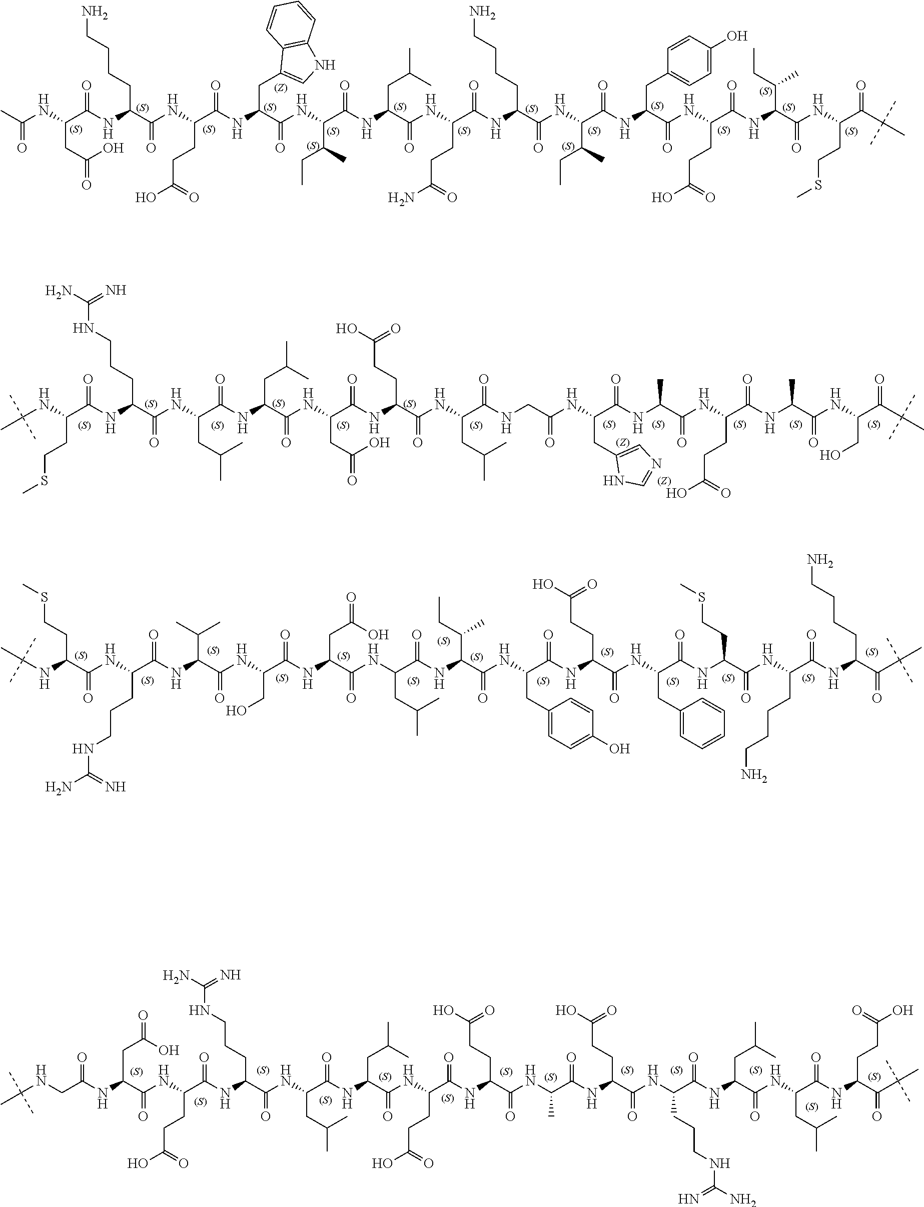

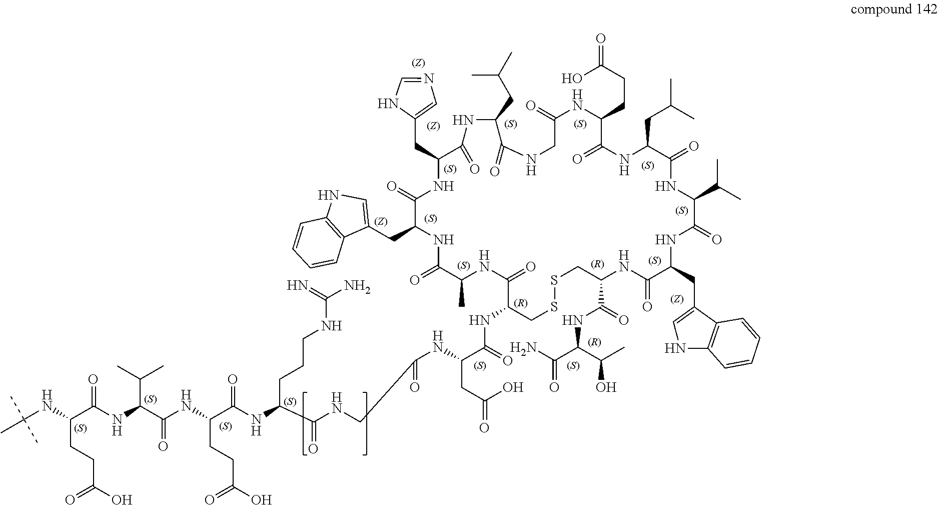

(SEQ ID NO: 37)
Ac-DKEWILQKIYEIMRLLDELGHAEASMRVSDLIYEFMKKGDERLLEEA
ERLLEEVER-GGGGGGGG-uABT-NH2

uABT disulfide cyclic peptides, Compounds 143, 144, 145, 146, and 147 were synthesized using the same method with compound 142.

0.50 mmol resin afforded compound 143 (Batch1: 11.2 mg, 83.9% purity; batch2: 8.10 mg, 76.4% purity; 0.44% yield) as a white solid. LCMS: RT=2.30 min, MS cal.: 8706.81, [M+5H]⁵⁺=1742.50, [M+6H]⁶⁺=1451.90, [M+7H]⁷⁺=1244.80, [M+8H]⁸⁺=1089.70, [M+9H]⁹⁺=968.40, [M+10H]¹⁰⁺=871.70.

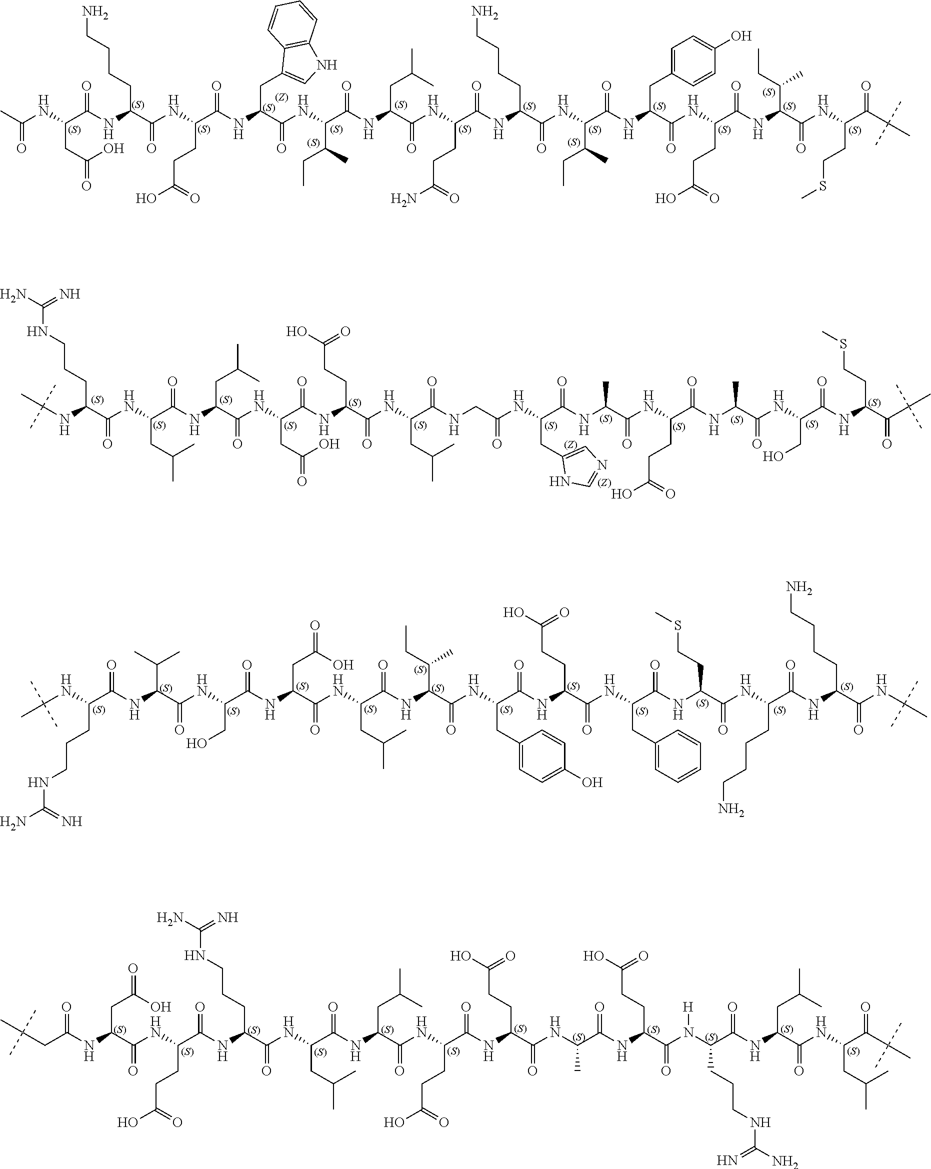

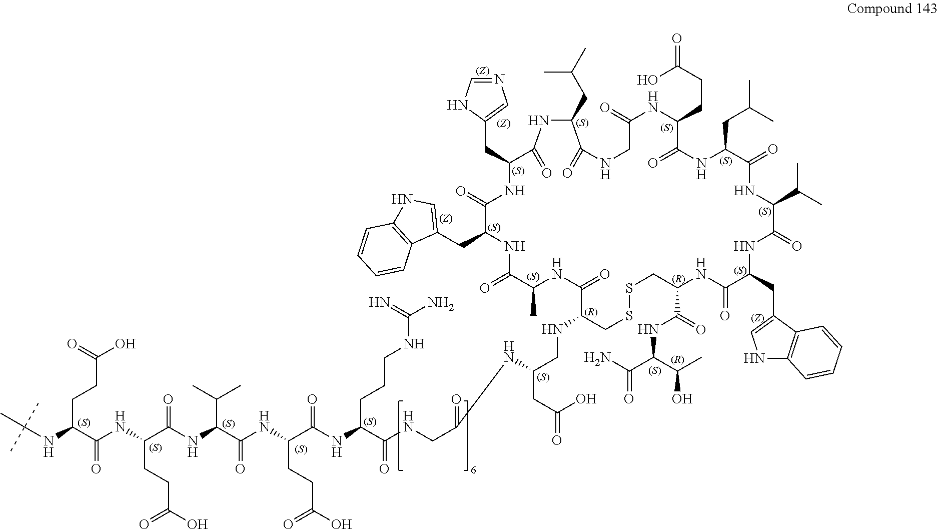

(SEQ ID NO: 31)
Ac-DKEWILQKIYEIMRLLDELGHAEASMRVSDLIYEFMKKGDERLLEEA
ERLLEEVER-GGGGGG-uABT-NH2

0.50 mmol resin afforded Compound 144 (19.7 mg, 84.3% purity, 0.46% yield) as a white solid. LCMS: RT=2.30 min, MS cal.: 8592.71, [M+5H]⁵⁺=1719.50, [M+6H]⁶⁺=1433.00, [M+7H]⁷⁺=1228.50, [M+8H]⁸⁺=1075.10, [M+9H]⁹⁺=955.80, [M+10H]¹⁰⁺=860.40, [M+11H]¹¹⁺=782.30

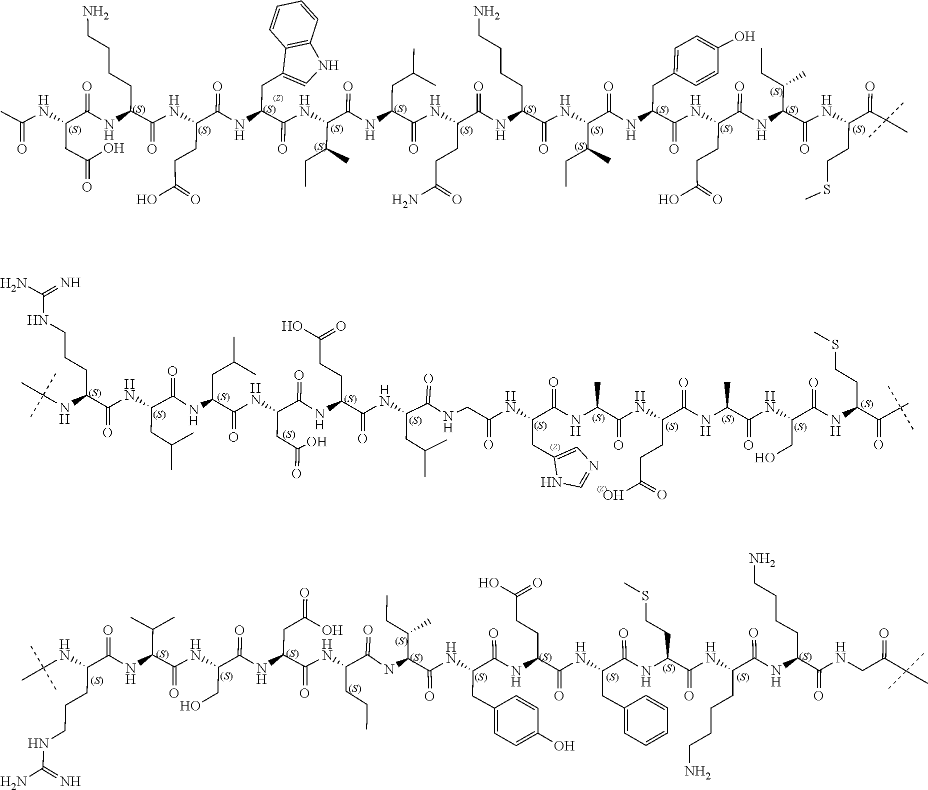

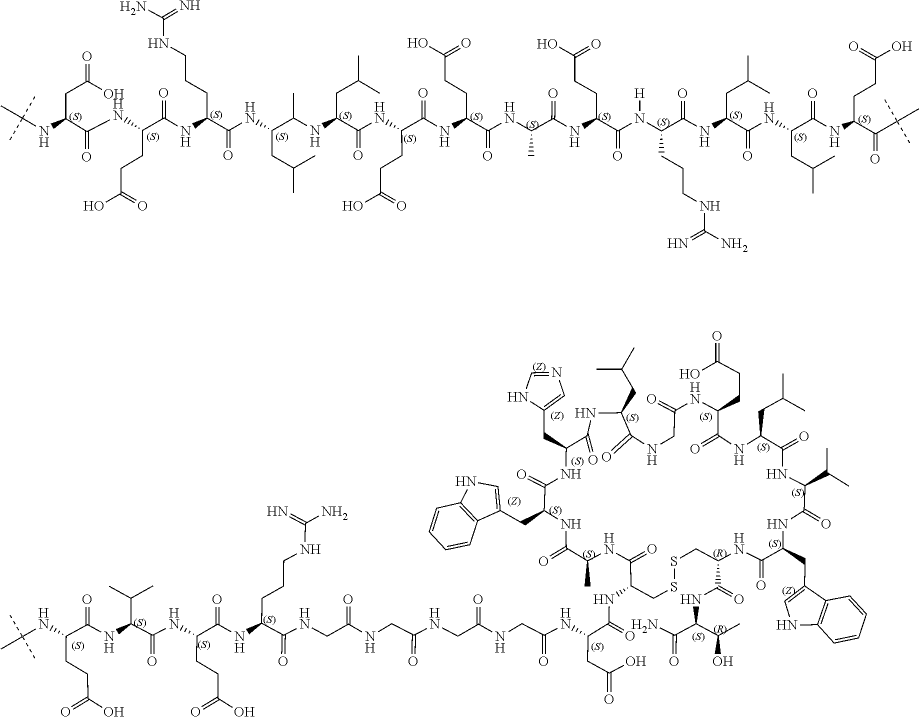

(SEQ ID NO: 32)
Ac-DKEWILQKIYEIMRLLDELGHAEASMRVSDLIYEFMKKGDERLLEEA
ERLLEEVER-GGGG-uABT-NH2

76.2% purity; 0.21% yield) as a white solid. LCMS: RT=2.30 min, MS cal.: 8478.60, [M+5H]⁵⁺=1696.90, [M+6H]⁶⁺=1414.30, [M+7H]⁷⁺=1212.30, [M+8H]⁸⁺=1060.90, [M+9H]⁹⁺=943.00, [M+10H]¹⁰⁺=848.80.

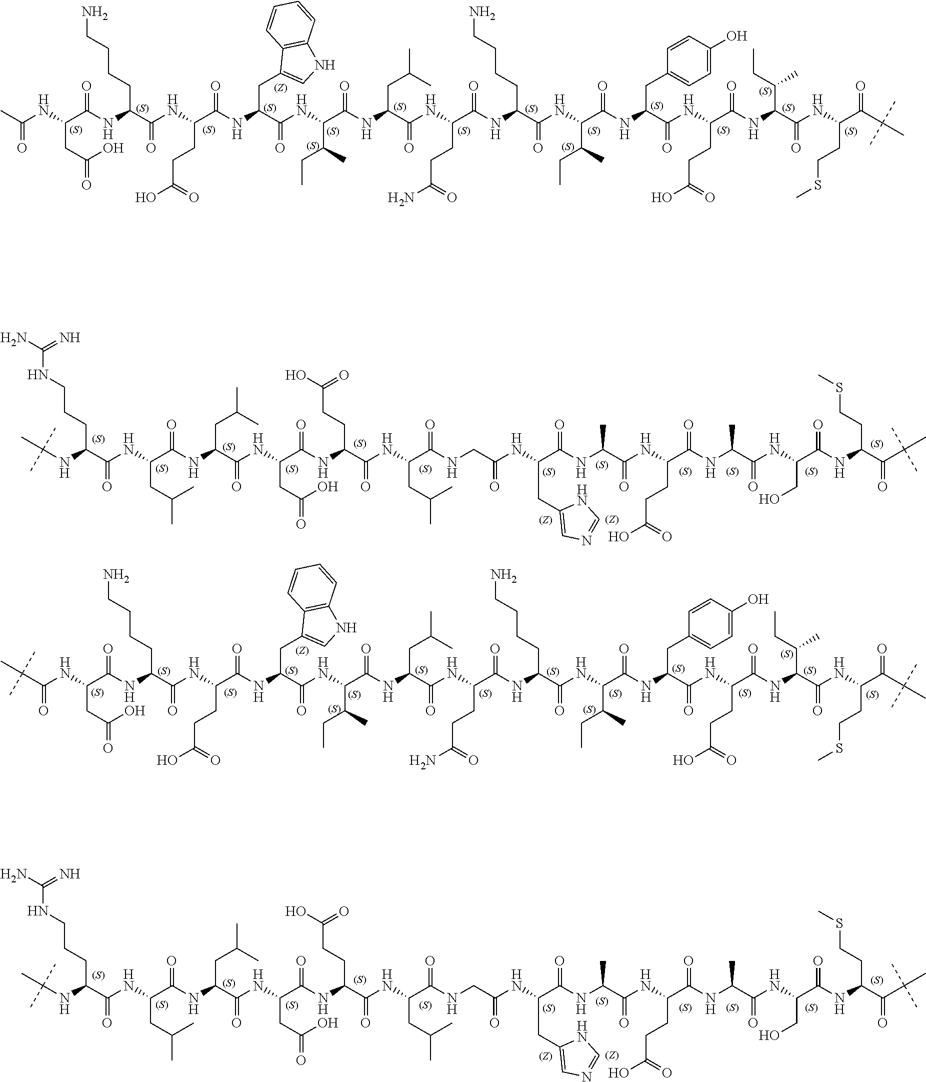

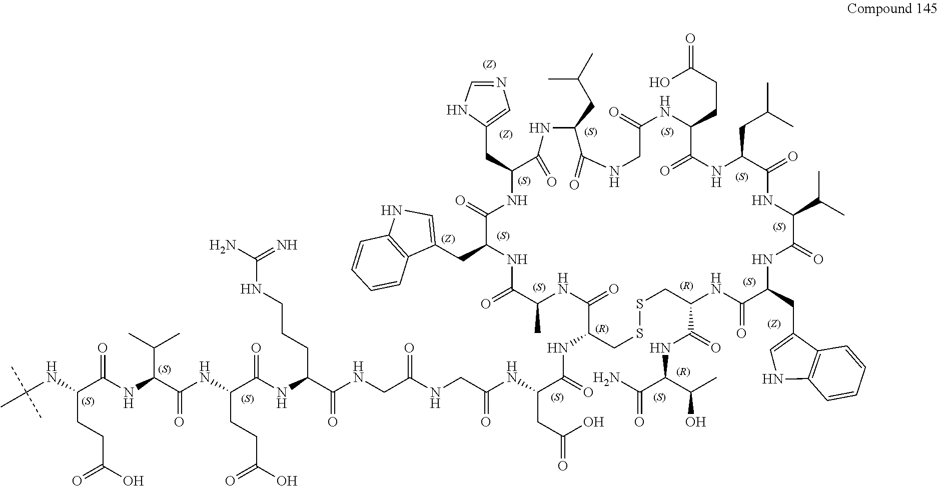

(SEQ ID NO: 38)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gly-Asp-
Cyclo[Cys-Ala-Trp-His-Leu-Gly-Glu-Leu-Val-Trp-
Cys]-Thr-NH2

0.50 mmol resin afforded compound 146 (SEQ ID NO:39) (Batch1: 6.2 mg, 75.6% purity; batch2: 8.5 mg, 79.1% purity; 0.32% yield) as a white solid. LCMS: RT=2.27 min, MS cal.: 9305.54, [M+5H]⁵⁺=1862.40, [M+6H]⁶⁺=1551.60, [M+7H]⁷⁺=1330.50, [M+8H]⁸⁺=1164.20, [M+9H]⁹⁺=1035.10, [M+10H]¹⁰⁺=931.50.

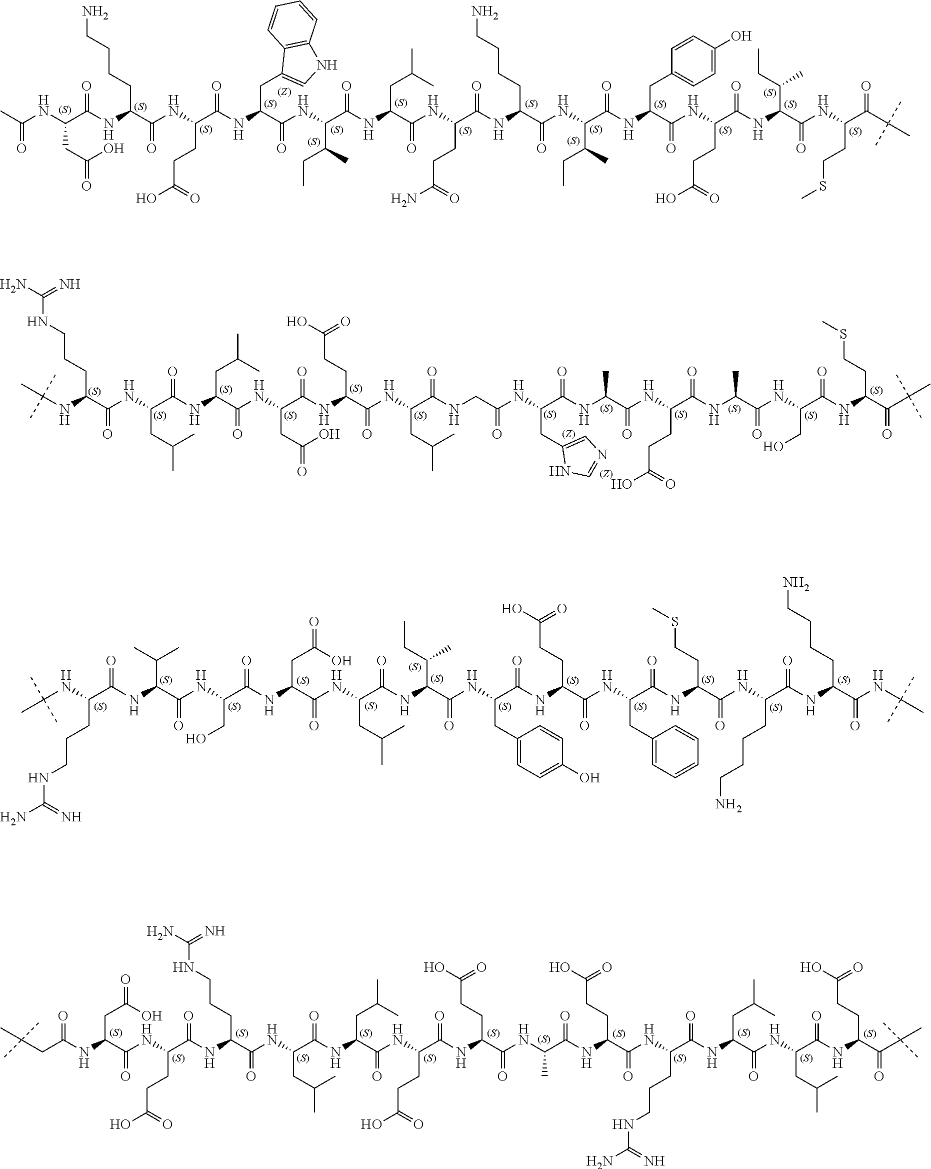

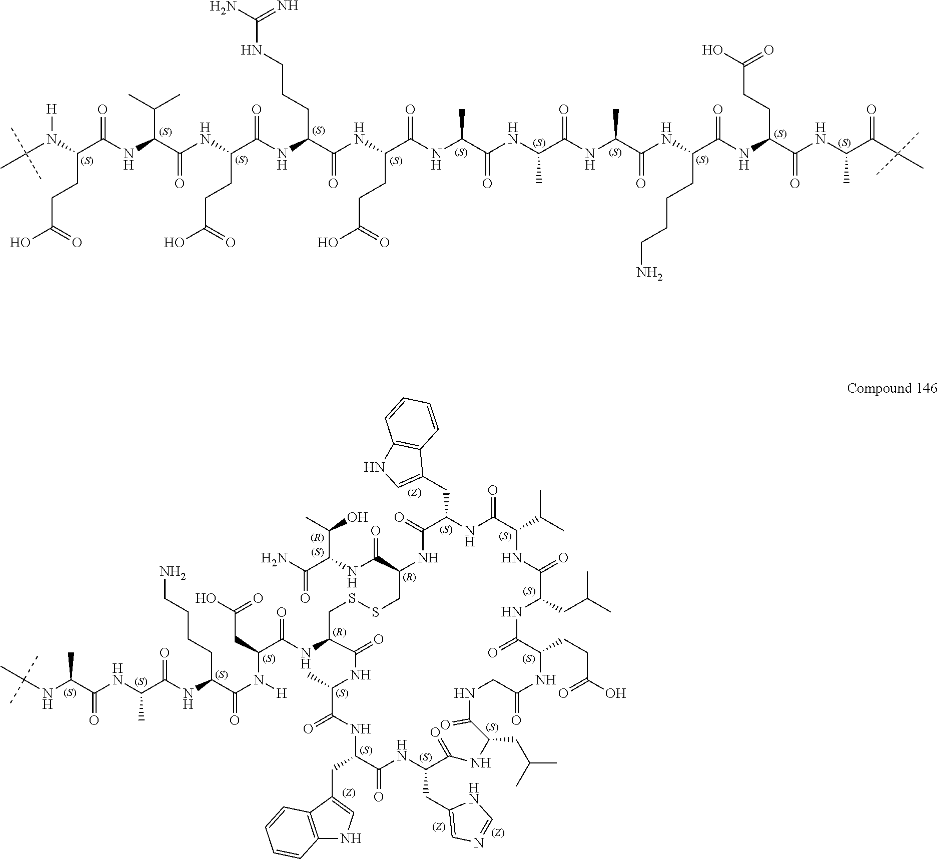

(SEQ ID NO: 39)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Glu-Ala-Ala-
Ala-Lys-Glu-Ala-Ala-Ala-Lys-Asp-Cyclo[Cys-Ala-Trp-
His-Leu-Gly-Glu-Leu-Val-Trp-Cys]-Thr-NH2

0.50 mmol resin afforded Compound 147 (SEQ ID NO:40) (Batch1: 14.8 mg, 83.3% purity; batch2: 10.8 mg, 63.10% purity; 0.56% yield) as a white solid. LCMS: RT=2.27 min, MS cal.: 9071.25, [M+5H]⁵⁺=1815.60, [M+6H]⁶⁺=1512.80, [M+7H]⁷⁺=1297.00, [M+8H]⁸⁺=1134.90, [M+9H]⁹⁺=1009.00, [M+10H]¹⁰⁺=908.10.

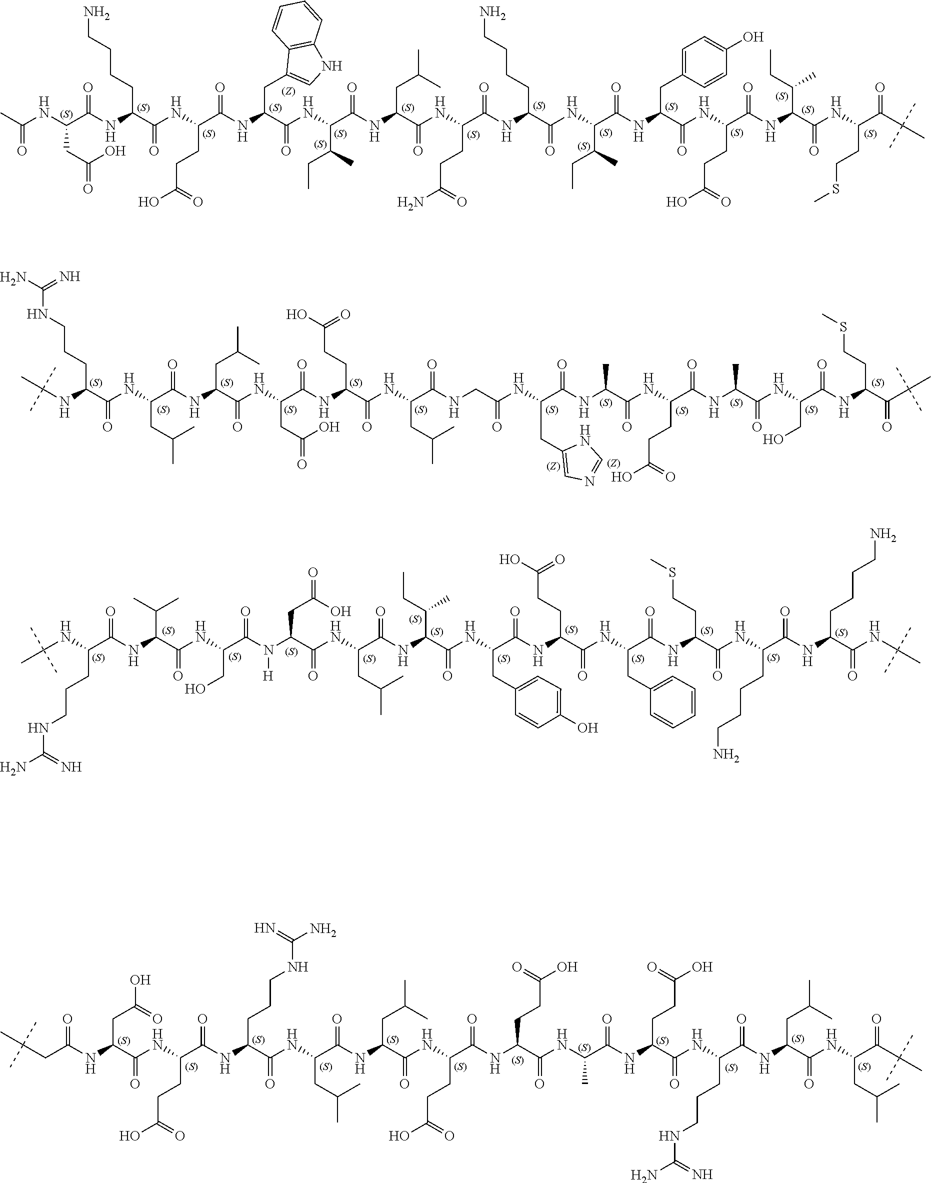

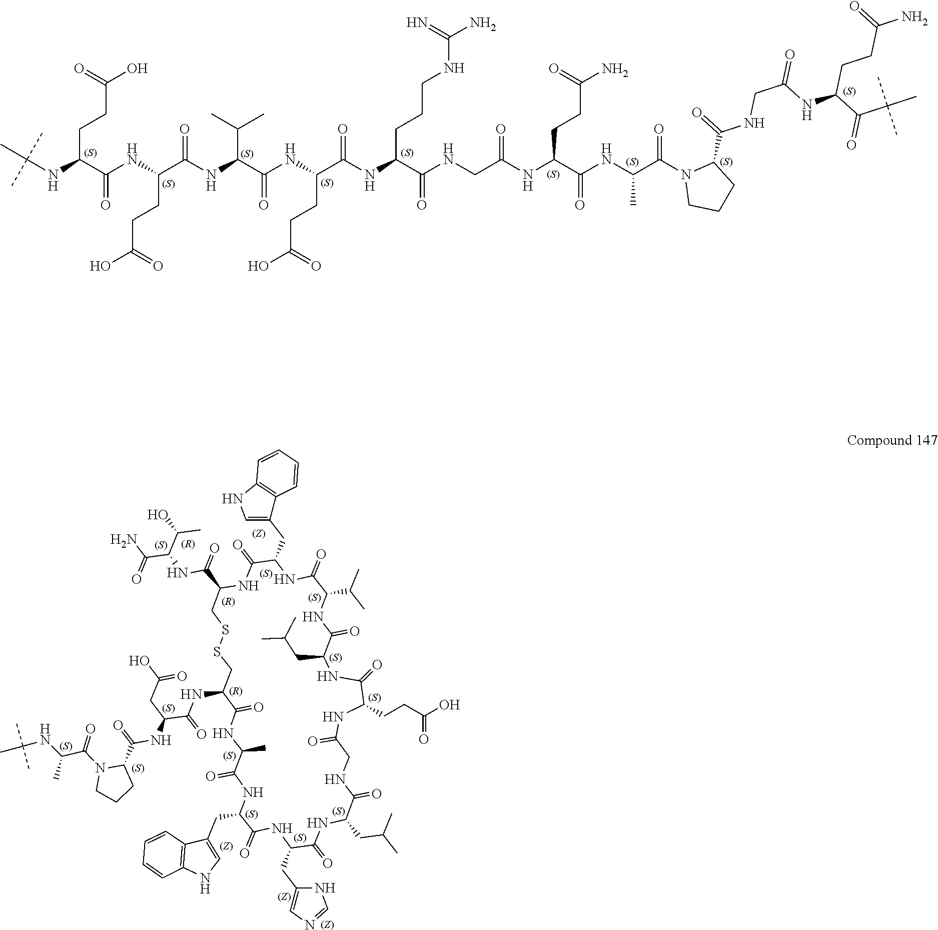

(SEQ ID NO: 40)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gln-Ala-
Pro-Gly-Gln-Ala-Pro-Asp-Cyclo[Cys-Ala-Trp-His-Leu-
Gly-Glu-Leu-Val-Trp-Cys]-Thr-NH2

### Example 10. Procedure for Preparation of Compound 148 (Linear Peptide+uABT)

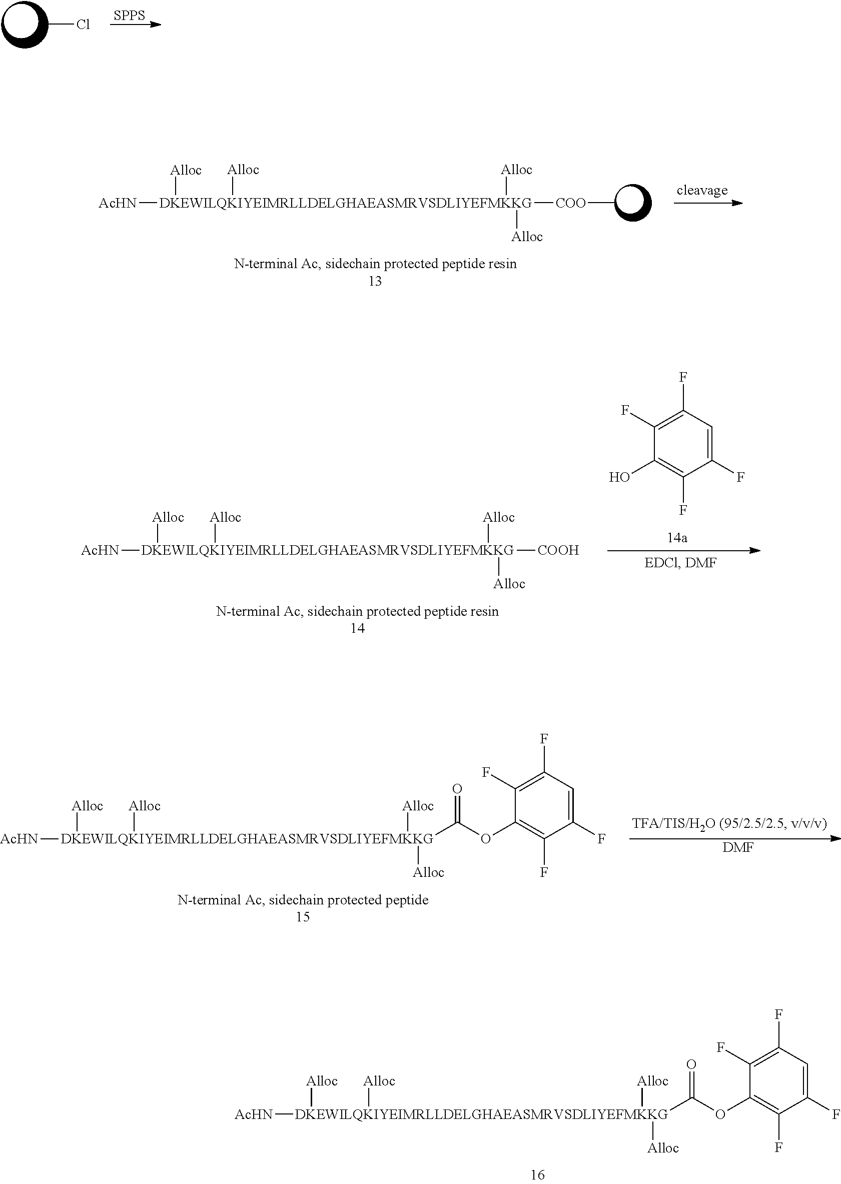

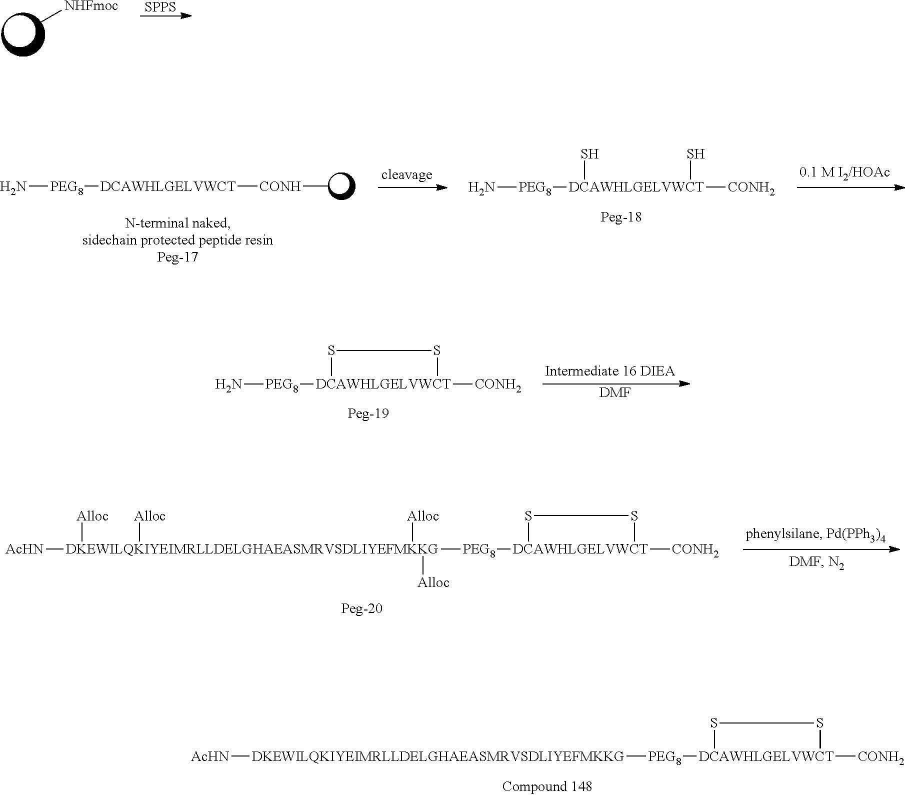

Preparation of Intermediate 14 (resin-bound peptide).

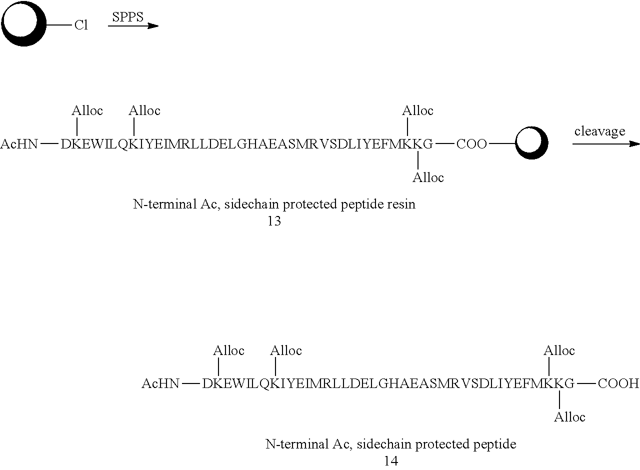

Peptide was synthesized using standard Fmoc chemistry (CTC resin).

Resin preparation: To the vessel containing CTC Resin (2.00 g, 2.00 mmol, 1.00 mmol/g) and Fmoc-Gly-OH (595.0 mg, 2.00 mmol, 1.00 equiv.) in DCM (20 mL) was added DIEA (4.00 equiv.) dropwise and mixed for 2 h with N₂ bubbling at 20° C. Then MeOH (2.0 mL) was added and bubbled with N₂ for another 30 min. The resin was washed with DMF (40 mL)*5, followed by the addition of 20% piperidine in DMF (40 mL) and bubbled with N₂ for 30 min at 20° C. for Fmoc deprotection. The mixture was filtered and the resin was washed with DMF (40 mL)*5 before proceeding to next step.

Coupling: A solution of Fmoc-Lys(Alloc)-OH (2.72 g, 3.00 equiv.), HBTU (2.16 g, 2.85 equiv.) in DMF (20 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (40 mL)*5.

Deprotection: 20% piperidine in DMF (40 mL) was added to the resin and the mixture was bubbled with N₂ for 30 min at 20° C. The deprotection reaction was monitored by ninhydrin test, if it showed blue or brownish red, the reaction was completed. The resin was then washed with DMF (40 mL) *5.

Coupling and Deprotection steps were repeated for the following amino acids elongation: Table 8.

Coupling: A solution of Fmoc-Ala-OH (1.87 g, 3.00 equiv.), HATU (2.16 g, 2.85 equiv.) in DMF (20 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (60 mL)*5.

The first coupling step was repeated for Fmoc deprotection.

The Coupling and Deprotection steps were repeated for the following amino acids elongation: Table 8.

Acetylation: A solution of Ac₂O/NMM/DMF (2/1/17, v/v/v, 40 mL) was added to the resin, the mixture was bubbled with N₂ for 20 min. The acetylation reaction was monitored by ninhydrin test. The resin was then washed with DMF (40 mL)*5, MeOH (40 mL)*3, then dried under reduced pressure.

TABLE 8
The list of amino acids and the corresponding reagents used on SPPS.

| # | Materials | Coupling reagents |
|---|---|---|
| 1 | Fmoc-Gly-OH (1.00 equiv.) | DIEA (4.00 equiv.) |
| 2 | Fmoc-Lys(Alloc)-OH (3.00 equiv.) | HBTU (2.85 equiv.) and DIEA (6.00 equiv.) |
| 3 | Fmoc-Lys(Alloc)-OH (3.00 equiv.) | HBTU (2.85 equiv.) and DIEA (6.00 equiv.) |
| 4 | Fmoc-Met-OH (3.00 equiv.) | HBTU (2.85 equiv.) and DIEA (6.00 equiv.) |
| 5 | Fmoc-Phe-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Tyr((Bu)-OH (3.00 equiv.) | HBTU (2.85 equiv.) and DIEA (6.00 equiv.) |
| 8 | Fmoc-Ile-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-Asp(OtBu)-OH (3.00 equiv.) | HBTU (2.85 equiv.) and DIEA (6.00 equiv.) |
| 11 | Fmoc-Ser(fBu)-OH (3.00 equiv.) | HBTU (2.85 equiv.) and DIEA (6.00 equiv.) |
| 12 | Fmoc-Val-OH (3.00 equiv.) | HBTU (2.85 equiv.) and DIEA (6.00 equiv.) |
| 13 | Fmoc-Arg(Pbf)-OH (3.00 equiv.) | HBTU (2.85 equiv.) and DIEA (6.00 equiv.) |
| 14 | Fmoc-Met-OH (3.00 equiv.) | HBTU (2.85 equiv.) and DIEA (6.00 equiv.) |
| 15 | Fmoc-Ser(tBu)-OH (3.00 equiv.) | HBTU (2.85 equiv.) and DIEA (6.00 equiv.) |
| 16 | Fmoc-Ala-OH (3.00 equiv.) | HBTU (2.85 equiv.) and DIEA (6.00 equiv.) |
| 17 | Fmoc-Glu(OtBu)-OH (3.00 equiv.) | HBTU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-His(Trt)-OH (3.00 equiv.) | HATU (2.85 equiv.) and DIEA (6.00 equiv.) |
| 20 | Fmoc-Gly-OH (3.00 equiv.) | HATU (2.85 equiv.) and DIEA (6.00 equiv.) |
| 21 | Fmoc-Leu-OH (3.00 equiv.) | HATU (2.85 equiv.) and DIEA (6.00 equiv.) |
| 22 | Fmoc-Glu(OtBu)-OH (3.00 equiv.) | HATU (2.85 equiv.) and DIEA (6.00 equiv.) |
| 23 | Fmoc-Asp(OtBu)-OH (3.00 equiv.) | HATU (2.85 equiv.) and DIEA (6.00 equiv.) |
| 24 | Fmoc-Leu-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Arg(Pbf)-OH (3.00 equiv.) | HATU (2.85 equiv.) and DIEA (6.00 equiv.) |
| 27 | Fmoc-Met-OH (3.00 equiv.) | HATU (2.85 equiv.) and DIEA (6.00 equiv.) |
| 28 | Fmoc-Ile-OH (3.00 equiv.) | HATU (2.85 equiv.) and DIEA (6.00 equiv.) |
| 29 | Fmoc-Glu(OtBu)-OH (3.00 equiv.) | HATU (2.85 equiv.) and DIEA (6.00 equiv.) |
| 30 | Fmoc-Tyr(fBu)-OH (3.00 equiv.) | HATU (2.85 equiv.) and DIEA (6.00 equiv.) |
| 31 | Fmoc-Ile-OH (3.00 equiv.) | HATU (2.85 equiv.) and DIEA (6.00 equiv.) |
| 32 | Fmoc-Lys(Alloc)-OH (3.00 equiv.) | HATU (2.85 equiv.) and DIEA (6.00 equiv.) |
| 33 | Fmoc-Gln(Trt)-OH (3.00 equiv.) | HATU (2.85 equiv.) and DIEA (6.00 equiv.) |
| 34 | Fmoc-Leu-OH (3.00 equiv.) | HATU (2.85 equiv.) and DIEA (6.00 equiv.) |
| 35 | Fmoc-Ile-OH (3.00 equiv.) | HATU (2.85 equiv.) and DIEA (6.00 equiv.) |
| 36 | Fmoc-Trp-OH (3.00 equiv.) | HATU (2.85 equiv.) and DIEA (6.00 equiv.) |
| 37 | Fmoc-Glu(OtBu)-OH (3.00 equiv.) | HATU (2.85 equiv.) and DIEA (6.00 equiv.) |
| 38 | Fmoc-Lys(Alloc)-OH (3.00 equiv.) | HATU (2.85 equiv.) and DIEA (6.00 equiv.) |
| 39 | Fmoc-Asp(OtBu)-OH (3.00 equiv.) | HATU (2.85 equiv.) and DIEA (6.00 equiv.) |
| 40 | Acetylation | Ac2O/NMM/DMF (2/1/17, v/v/v) |

Peptide Cleavage. Cleavage solution (HFIP/DCM, 1/4, v/v, 200 mL) was added to the flask containing the side-chain protected resin-bound peptide (intermediate 13 (SEQ ID NO:12), 15.1 g, 2.00 mmol) at room temperature and stirred for 1 h.

After filtration, the filtrate was collected. The filtrate was concentrated under reduced pressure.

The residue was dissolved in MeCN/H₂O (4/6, v/v, 500 mL) and lyophilized to afford intermediate 14 (SEQ ID NO:12) (12.0 g, crude) as a white solid.

### Preparation of Intermediate 15 (SEQ ID NO:13).

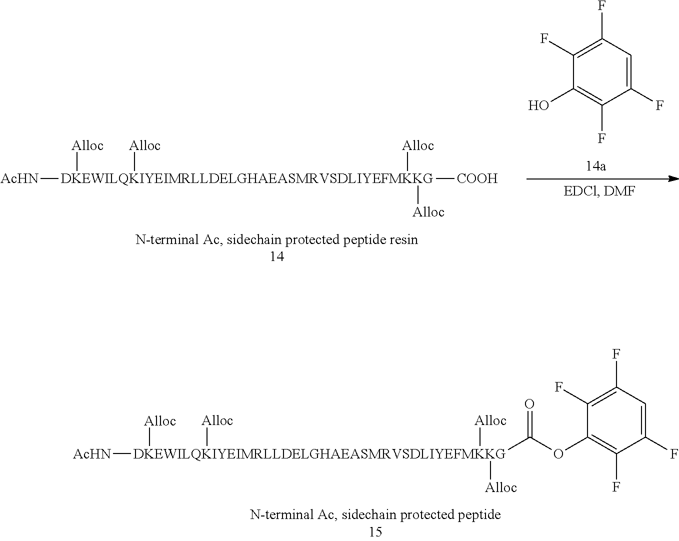

To a mixture of intermediate 14 (12.00 g, crude, 1.00 equiv.) and 14a (3.32 g, 20.00 mmol, 10.00 equiv.) in DMF (100 mL) was added EDCI (2.30 g, 12 mmol, 6.00 equiv.) at 20° C. The reaction mixture was stirred at 20° C. for 12 h. Then the mixture was precipitated with 0.1 M aq. HCl (cold, 1000 mL). After filtration, the solid was dried by lyophilization to afford intermediate 15 (SEQ ID NO:13) (18.30 g, crude) as a white solid.

Preparation of intermediate 16 (SEQ ID NO:43).

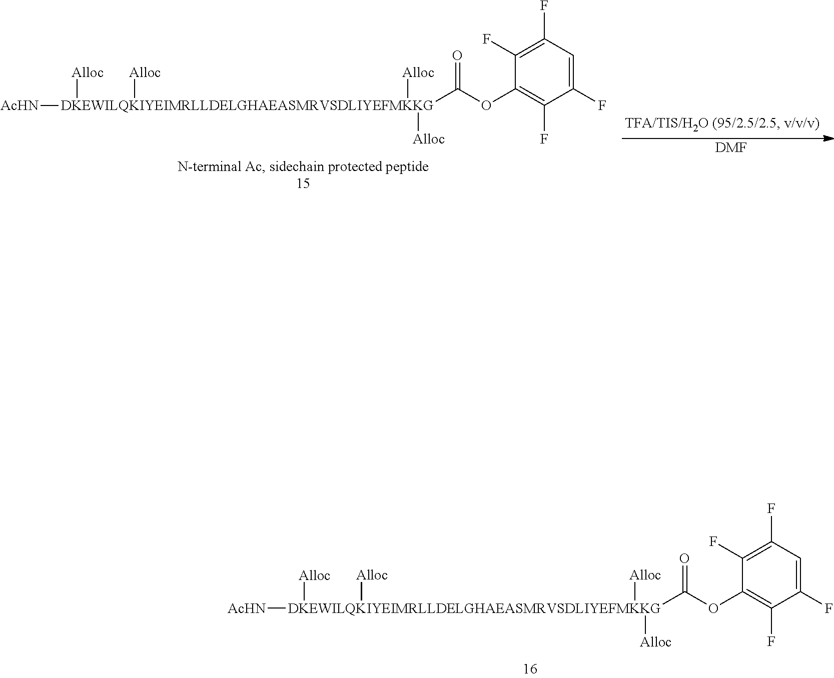

A solution of intermediate 15 (SEQ ID NO:13) (18.30 g, crude) in TFA/TIS/H₂O (95/2.5/2.5, v/v/v, 200 mL) was stirred at 20° C. for 2 h. The mixture was precipitated with cold isopropyl ether (800 mL). After filtration, the solid was washed with isopropyl ether (500 mL) twice, dried under reduced pressure at room temperature. The solid was purified by prep-HPLC (A: 0.075% TFA/H₂O; B: MeCN), followed by lyophilization to afford intermediate 16 (SEQ ID NO:43) (1.08 g, 74.0% purity, 7.64% yield, impurity came from the hydrolysis of intermediate 16) as a white solid. LCMS: RT=1.33 min, MS cal.: 5226.85, mass observed: [M+3H]³⁺=1743.14, [M+4H]⁴⁺=1307.75.

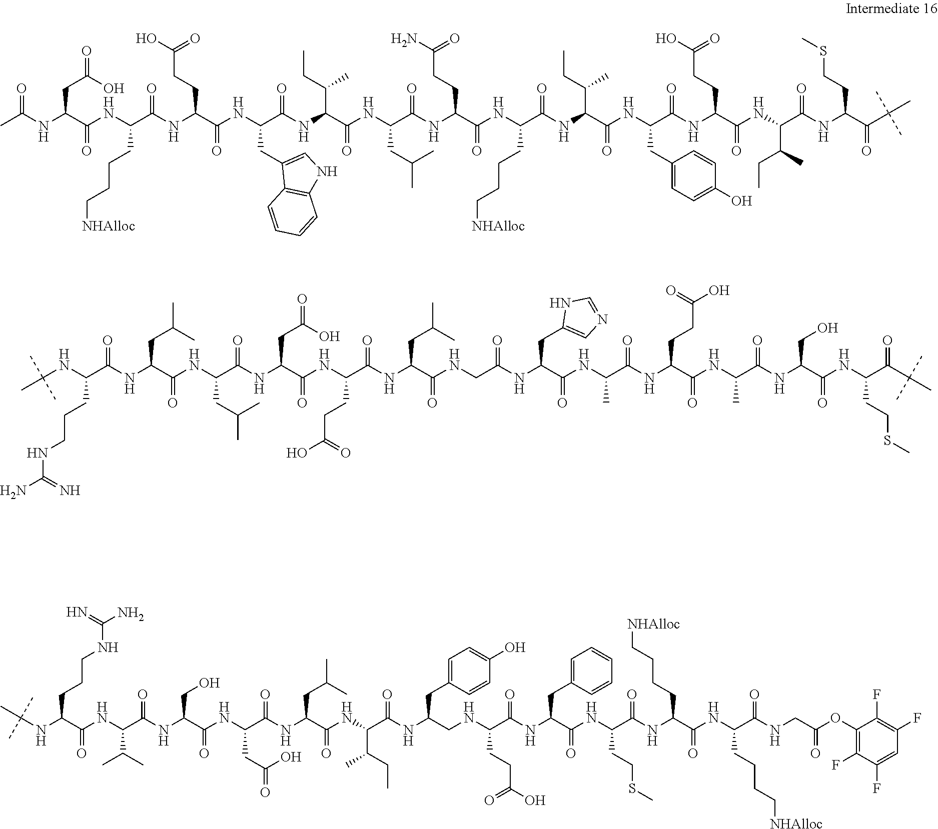

Preparation of Peg-19 (disulfide cyclic peptide).

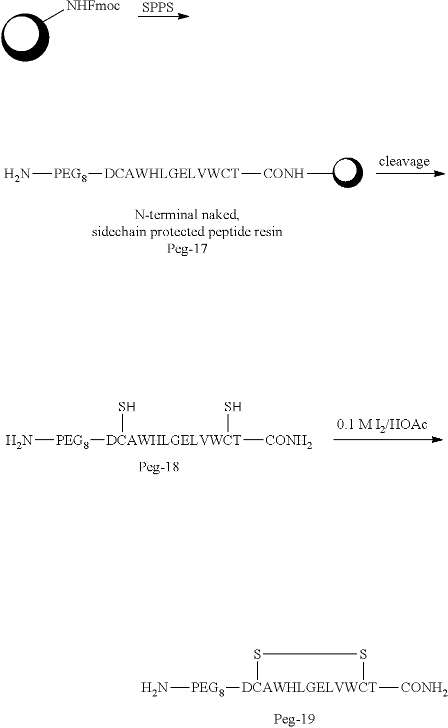

Peptide was synthesized using standard Fmoc chemistry (Rink AM resin).

Resin preparation: To the vessel containing Rink Amide AM resin (6.25 g, 2.00 mmol, 0.32 mmol/g) and DMF (80 mL) was bubbled with N₂ for 2 h at 20° C. Then 20% piperidine in DMF (80 mL) was added and the mixture was bubbled with N₂ for 30 min at 20° C. The mixture was filtered and washed with DMF (40 mL)*5 before proceeding to next step.

Coupling: A solution of Fmoc-Thr(tBu)-OH (2.38 g, 6.00 mmol, 3.00 equiv.), HBTU (2.19 g, 5.70 mmol, 2.85 equiv.) in DMF (40 mL) was added to the resin with N₂ bubbling. Then DIEA (1.54 g, 12.00 mmol, 6.00 equiv.) was added to the mixture dropwise and bubbled with N₂ for 30 min at 20° C. The coupling reaction was monitored by ninhydrin test. The resin was then washed with DMF (80 mL)*5.

Deprotection: 20% piperidine in DMF (80 mL) was added to the resin and the mixture was bubbled with N₂ for 30 min at 20° C. The deprotection reaction was monitored by ninhydrin test. The resin was then washed with DMF (80 mL)*5.

The Coupling and Deprotection steps were repeated for the following amino acids elongation: Number #2-12, Table 2.

After all the steps were completed, the resin was washed with DMF (80 mL)*5, MeOH (80 mL)*5, then dried under reduced pressure to afford resin-bound peptide intermediate Peg-17 (SEQ ID NO:44) (Rink AM resin, 12.72 g, 2.00 mmol).

TABLE 9
The list of amino acids and the corresponding reagents used on SPPS.

| # | Materials | Coupling reagents |
|---|---|---|
| 1 | Fmoc-Thr(OtBu)-OH (3.00 equiv.) | HBTU (2.85 equiv.) and DIEA (6.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-His(Trt)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |
| 14 | Fmoc-PEG8-CH2CH2COOH (1.50 equiv.) | HBTU (1.42 equiv.) and DIEA (3.00 equiv.) |

Peptide cleavage, cyclization and purification. Cleavage solution (TFA/TIS/H₂O, 95/2.5/2.5, v/v/v, 240 mL) was added to the flask containing the side-chain protected resin-bound peptide intermediate 5 (Rink AM resin, 12.72 g, 2.00 mmol) at room temperature and stirred for 2 h.

After filtration, the filtrate was collected. The filtrate was precipitated with cold isopropyl ether (1.5 L). After filtration, the solid was washed with isopropyl ether (500 mL) twice, and the crude peptide was dried under reduced pressure for 2 h to afford intermediate Peg-18 (SEQ ID NO:45) (4.52 g, crude) as a white solid.

To a mixture of intermediate Peg-18 (SEQ ID NO:45) (4.52 g, crude) in HOAc/MeCN/H₂O (4/3/3, v/v/v, 2.0 L) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford intermediate Peg-19 (SEQ ID NO:46) (720.0 mg, 96.0% purity, 18.4% yield) as a white solid. LCMS: RT=0.90 min, MS cal.: 1953.24, mass observed: [M+2H]²⁺=977.04, [M+3H]³⁺=651.6.

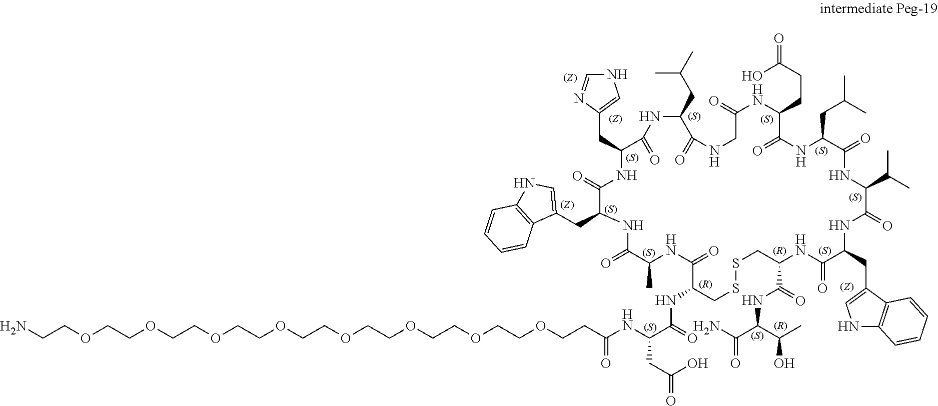

Preparation of intermediate Peg-20 (fragments coupling).

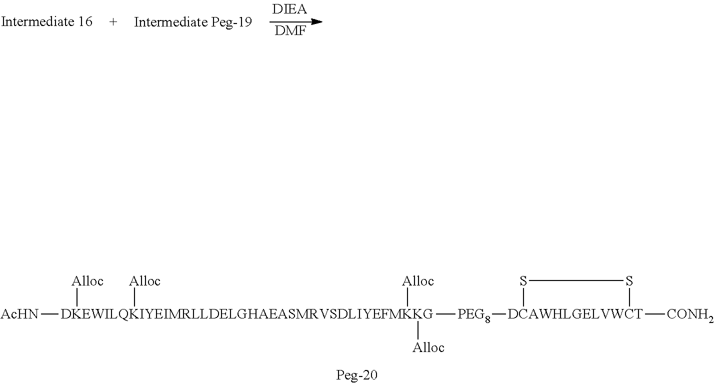

To a mixture of intermediate 16 (SEQ ID NO:43) (110.0 mg, 21.0 mol, 1.00 equiv.) and intermediate Peg-19 (SEQ ID NO:46) (87.8 mg, 57.4 mol, 1.00 equiv.) in DMF (3 mL) was added DIEA (74.1 mg, 573.9 mol, 10.0 equiv.) at 20° C. The mixture was stirred at 20° C. for 2 h. Monitored by LCMS until intermediate Peg-19 was consumed. The mixture was acidified by 1 M HCl to pH=5 and purified by prep-HPLC (A: 0.075% TFA/H₂O, B: MeCN) directly, then lyophilized to afford intermediate Peg-20 (SEQ ID NO:47) (70.0 mg, 86.7% purity, 41.1% yield) as a white solid. TOF: RT=1.57 min, MS cal.: 7014.01, mass observed: [M+4H]⁴⁺=1754.07, [M+5H]⁵⁺=1403.66, [M+6H]⁶⁺=1169.88.

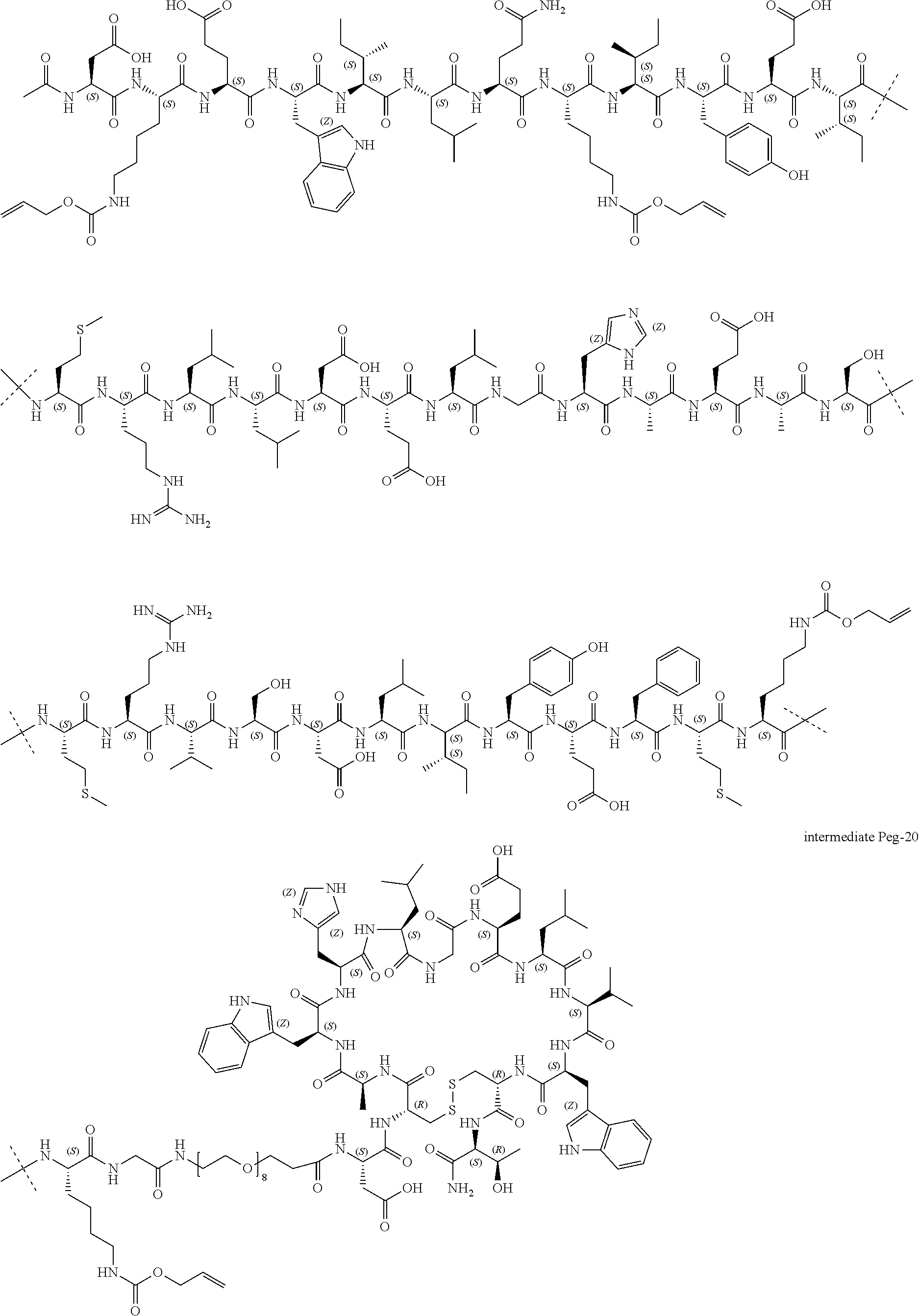

Preparation of Compound 148 (de-Alloc).

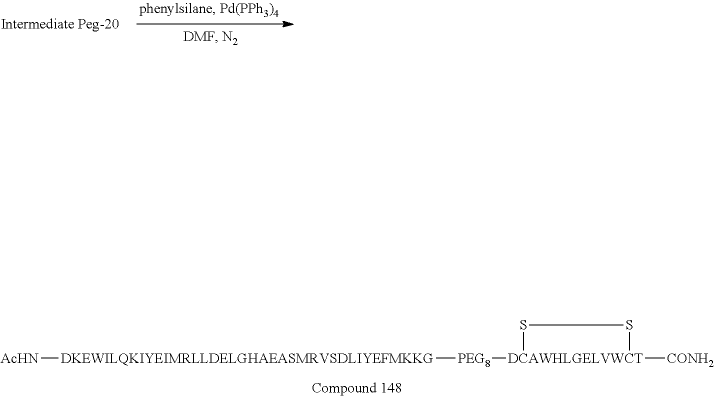

A solution of intermediate Peg-20 (SEQ ID NO:47) (70.0 mg, 9.98 mol, 1.00 equiv.), phenylsilane (10.8 mg, 99.8 mol, 12.0 uL, 10.00 equiv.), Pd(PPh₃)₄ (5.76 mg, 4.99 mol, 0.50 equiv.) in DMF (1 mL) was stirred under N₂ atmosphere at 20° C. for 0.5 h. The mixture was purified by prep-HPLC (A: 0.075% TFA/H₂O, B: MeCN) directly, then lyophilized to afford Compound 148 (SEQ ID NO:48) (24 mg, 86.5% purity, 31.1% yield) as a white solid. LCMS: RT=1.02 min, MS cal.: 6677.72, mass observed: [M+4H]⁴⁺+=1670.10, [M+5H]⁵⁺=1136.50, [M+6H]⁶⁺=1114.00.

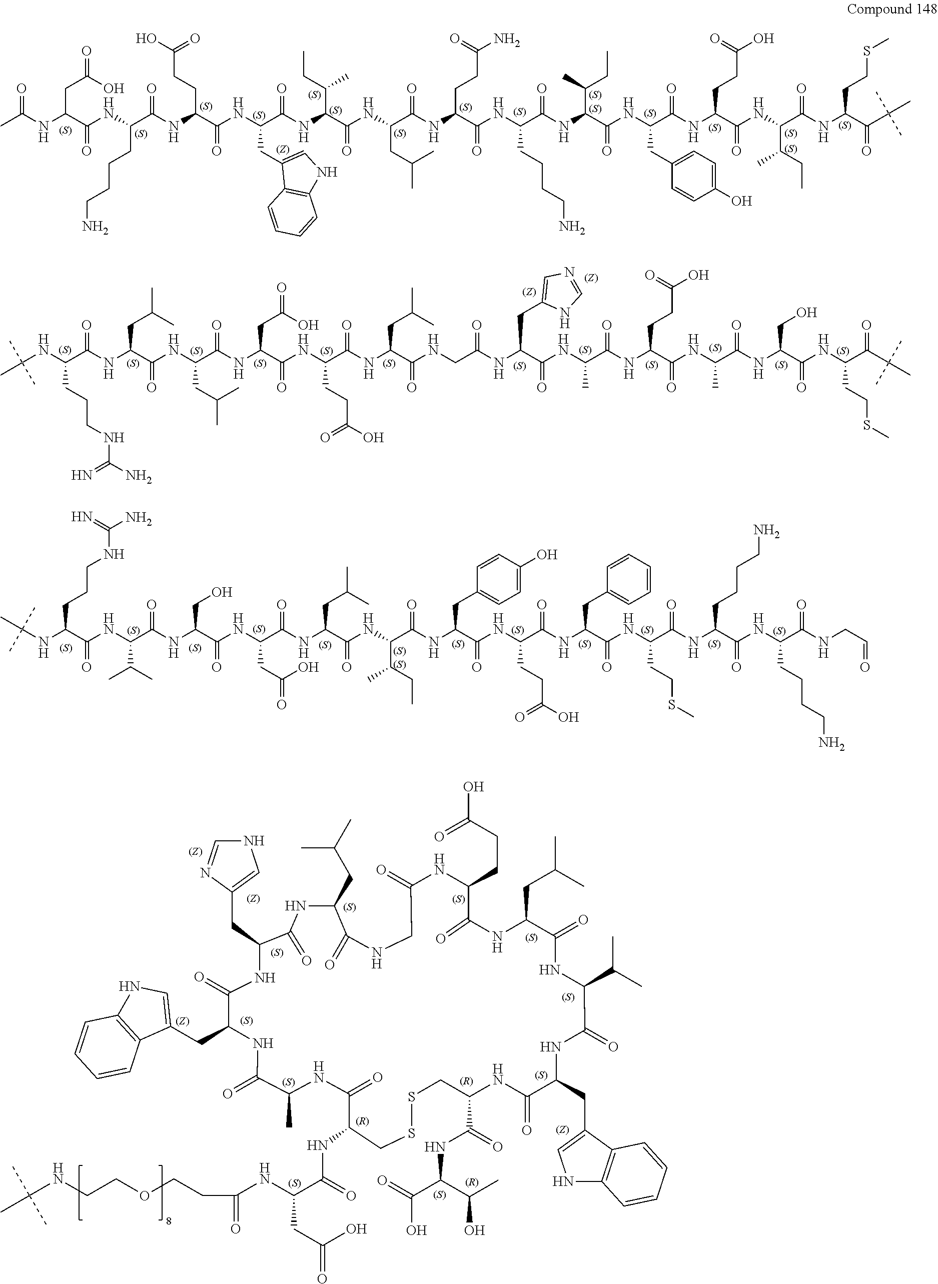

Compounds 149, 150, 151, 152, 153, 154, 155, and 156 were synthesized using the same method as Compound 148 (SEQ ID NO:48).

0.20 mmol resin afforded Compound 149 (34.0 mg, 96.1% purity, 2.3% total yield) as a white solid. LCMS: RT=1.11 min, MS cal.: 7101.22, mass observed: [M+4H]⁴⁺=1776.24, [M+5H]⁵⁺=1421.17, [M+6H]⁶⁺=1184.40, [M+7H]⁷⁺=1015.27.

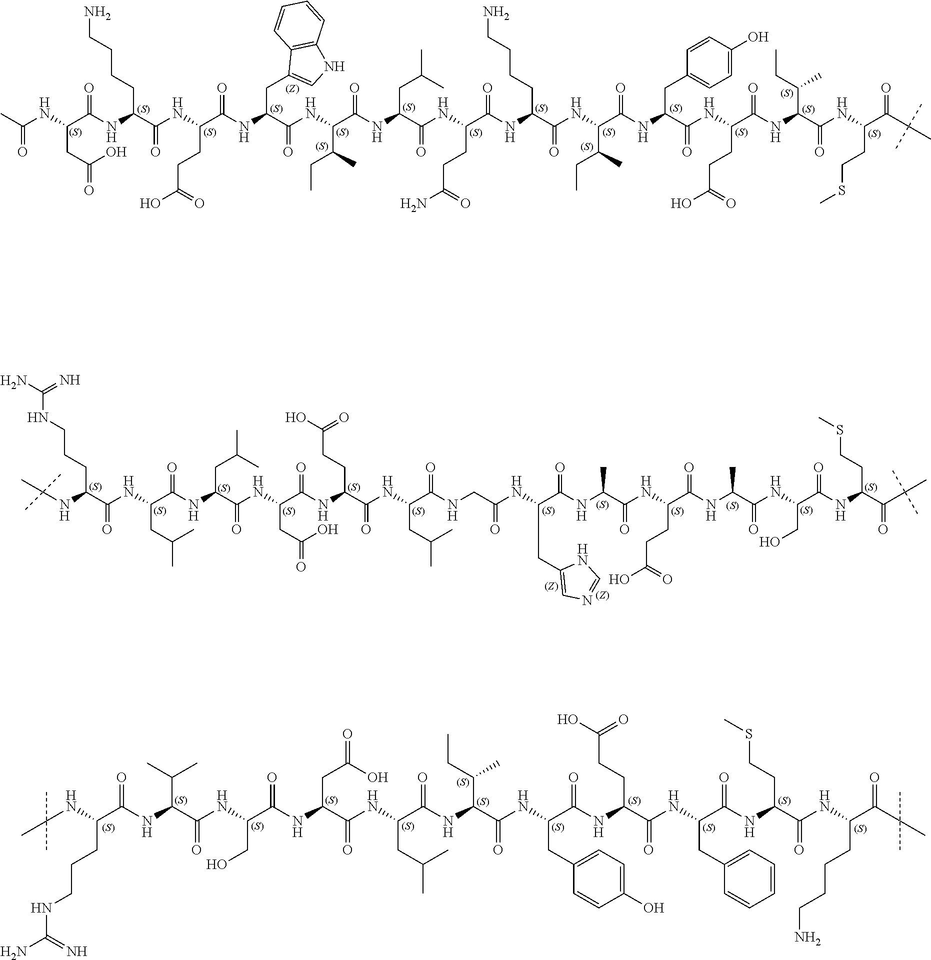

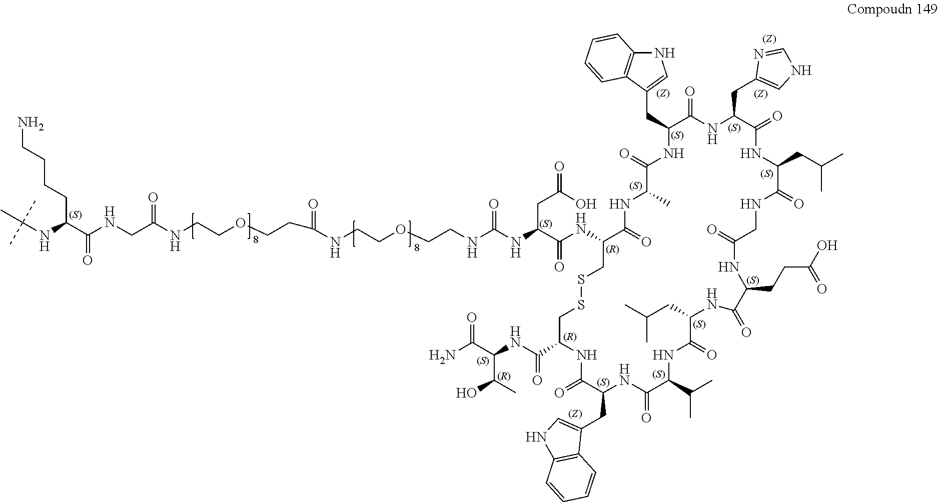

0.20 mmol resin afforded Compound 150 (7.1 mg, 95.1% purity, 0.49% total yield) as a white solid. LCMS: RT=1.03 min, MS cal.: 7186.32, mass observed: [M+4H]⁴⁺=1797.23, [M+5H]⁵⁺=1438.06, [M+6H]⁶⁺=1198.71, [M+7H]⁷⁺=1027.18.

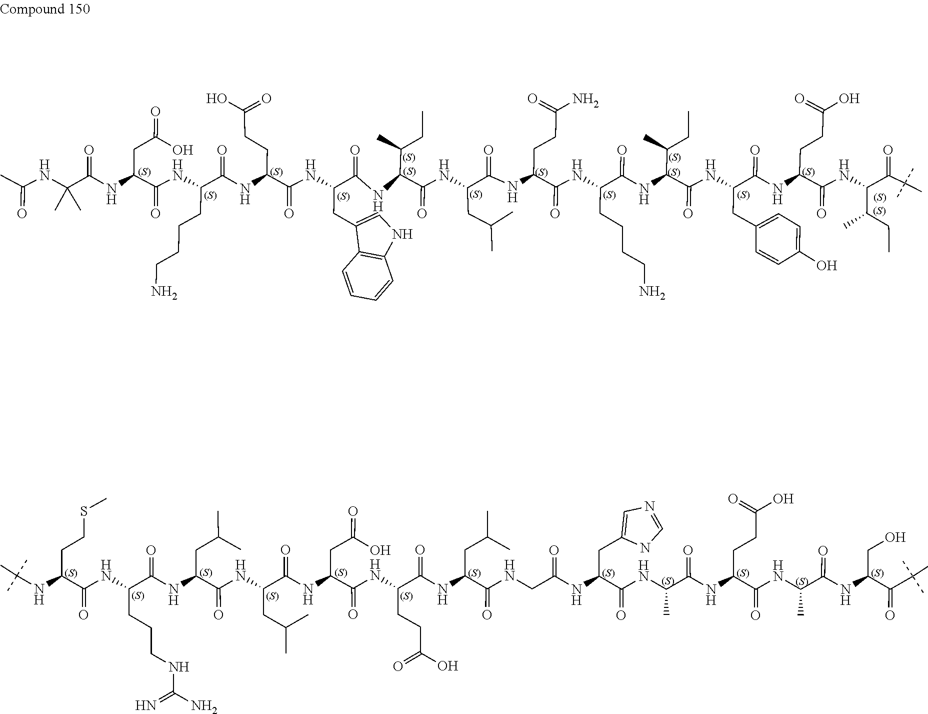

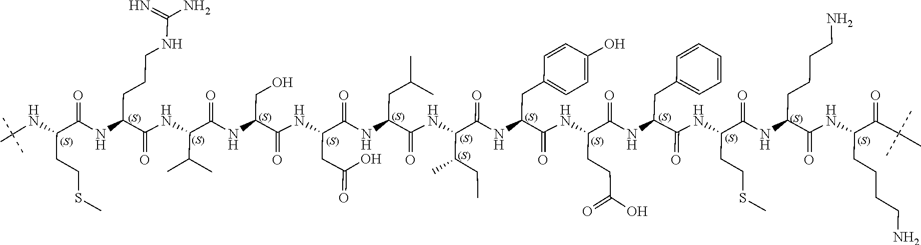

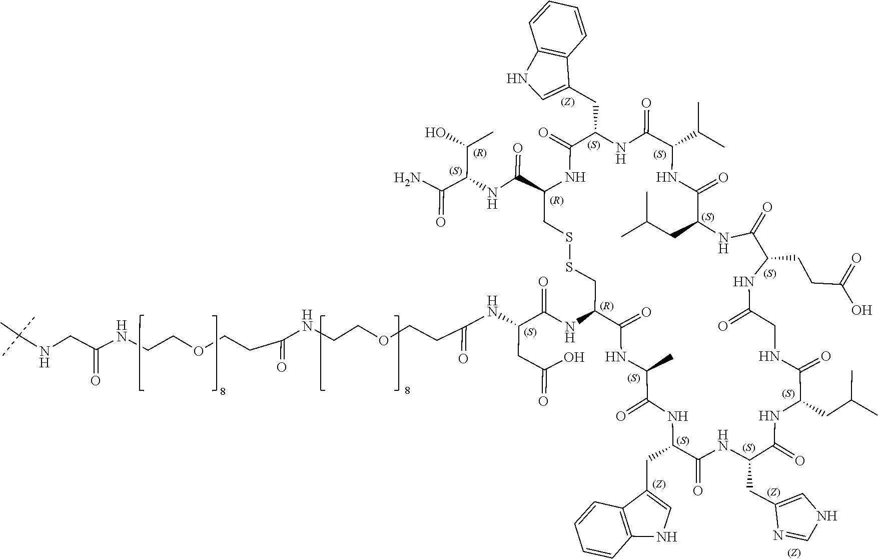

0.20 mmol resin afforded Compound 151 (17.8 mg, 93.3% purity, 1.81% total yield) as a white solid. LCMS: RT=1.03 min, MS cal.: 4906.68, mass observed: [M+3H]³⁺=1636.17, [M+4H]⁴⁺+=1227.36, [M+5H]⁵⁺=982.06, [M+6H]⁶⁺=818.42.

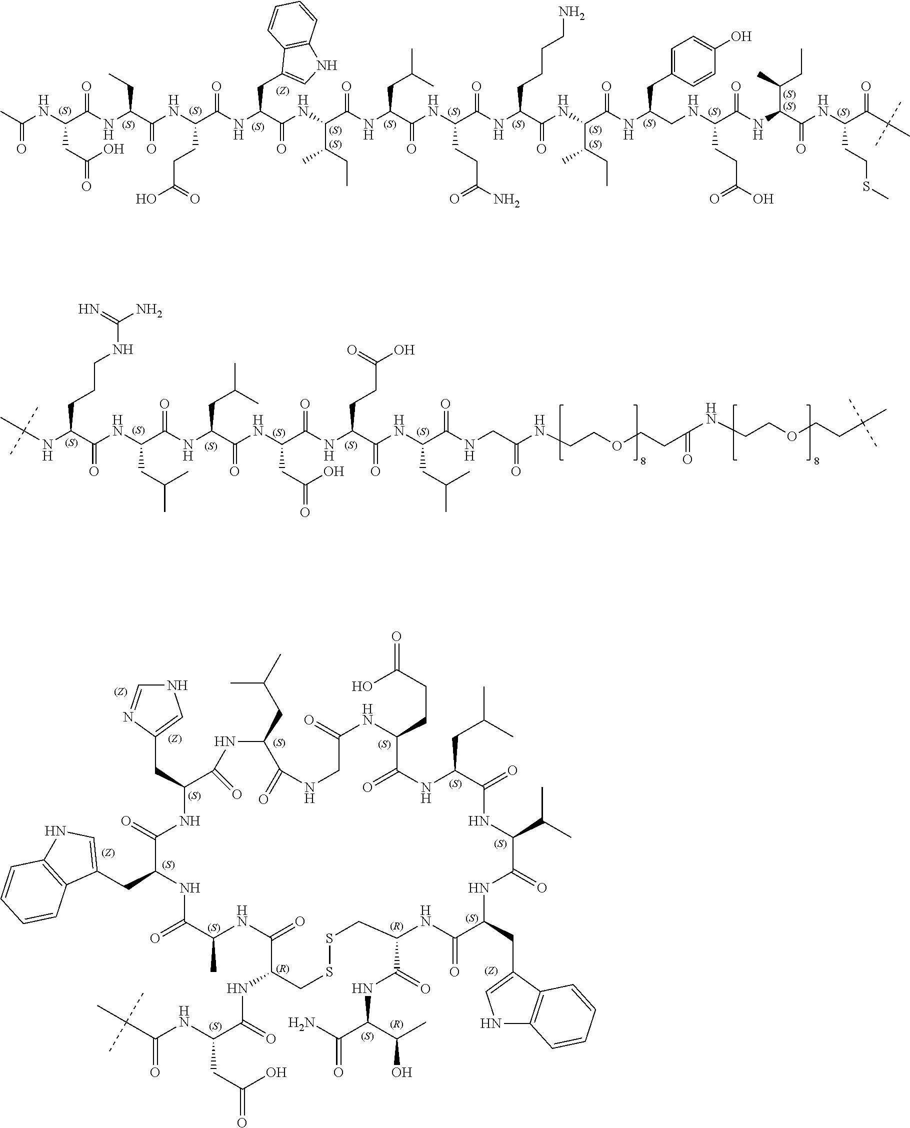

0.20 mmol resin afforded Compound 152 (17.8 mg, 93.3% purity, 1.81% total yield) as a white solid. LCMS: RT=1.03 min, MS cal.: 5402.17, mass observed: [M+3H]³⁺=1801.63, [M+4H]⁴⁺+=1351.39, [M+5H]⁵⁺=1801.39, [M+6H]⁶⁺=901.16.

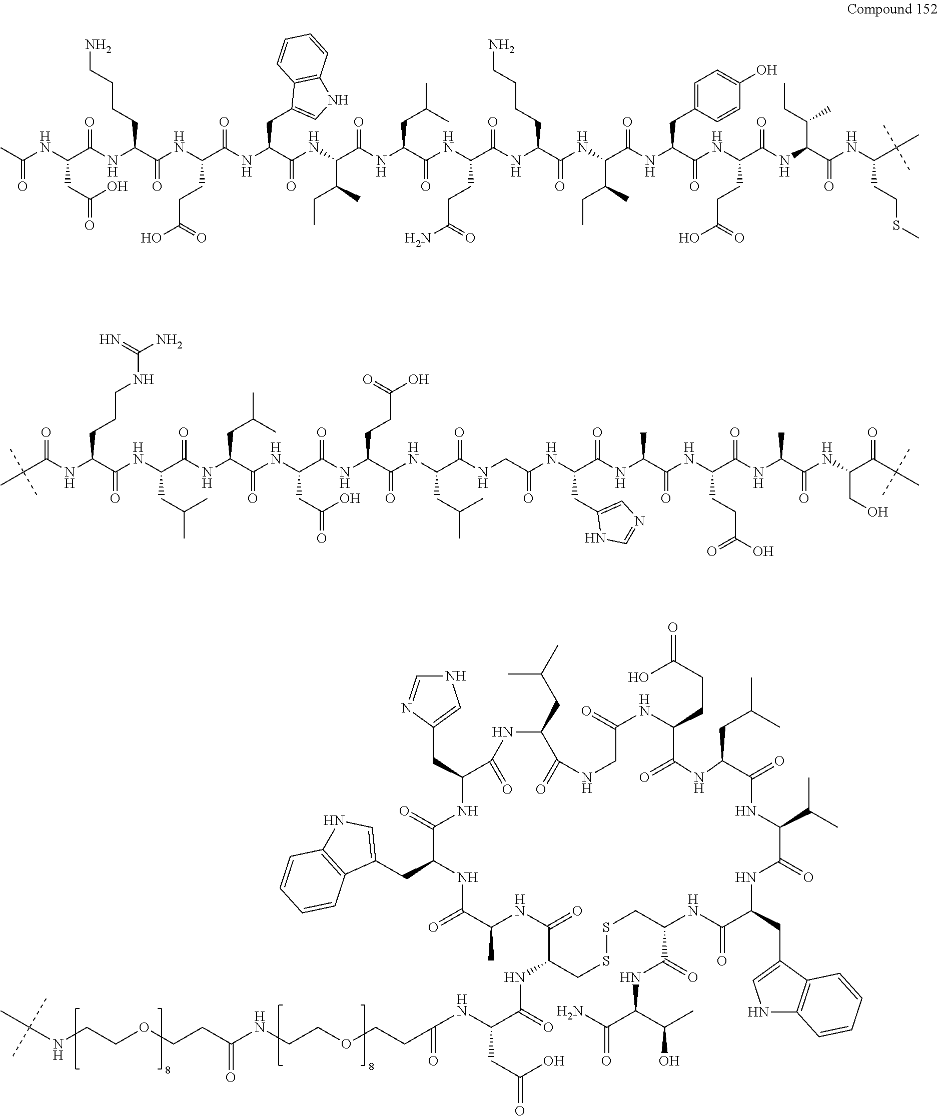

0.20 mmol resin afforded Compound 153 (18.1 mg, 87.4% purity, 1.81% yield in total) as a white solid. LCMS: RT=1.03 min, MS cal.: 5875.76, mass observed: [M+3H]³⁺=1959.32, [M+4H]⁴⁺+=1469.49, [M+5H]⁵⁺=1176.04, [M+6H]⁶⁺=979.99.

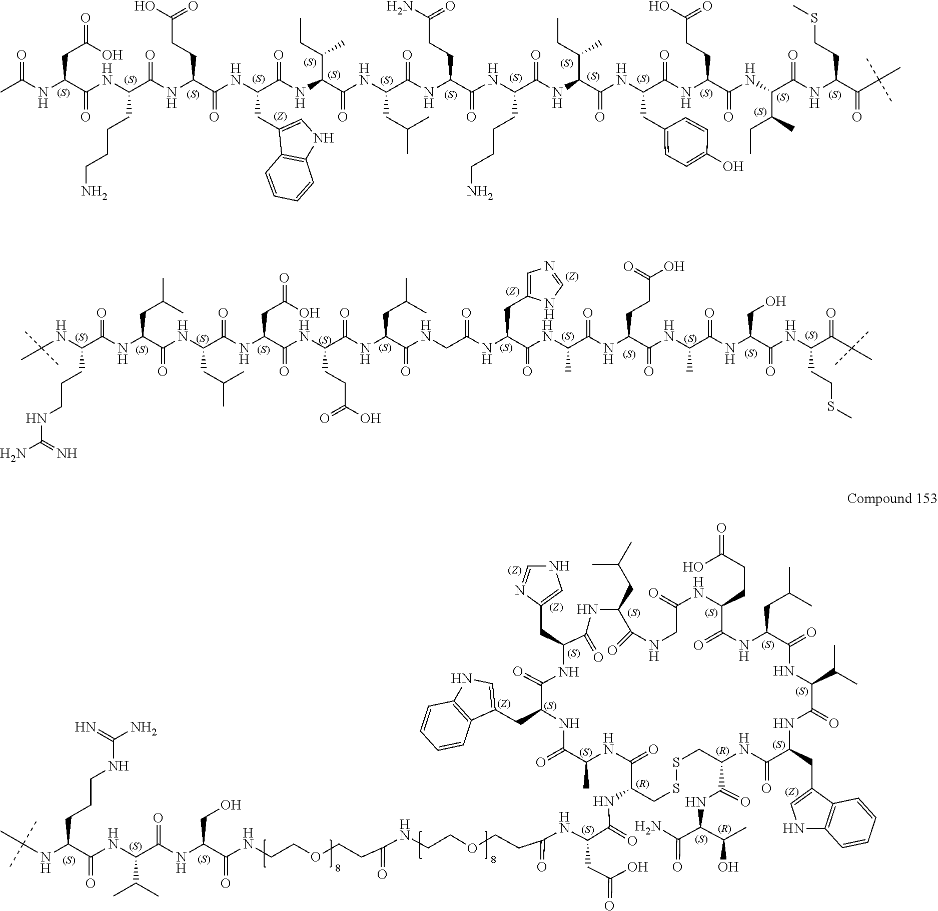

0.20 mmol resin afforded Compound 154 (Batch 1, 10.7 mg, 93.7% purity; Batch 2, 10.9 mg, 92.1% purity; 1.6% total yield) as a white solid. LCMS: RT=1.03 min, MS cal.: 6509.45, mass observed: [M+4H]⁴⁺+=1628.04, [M+6H]⁶⁺=1085.91, [M+7H]⁷⁺=930.43.

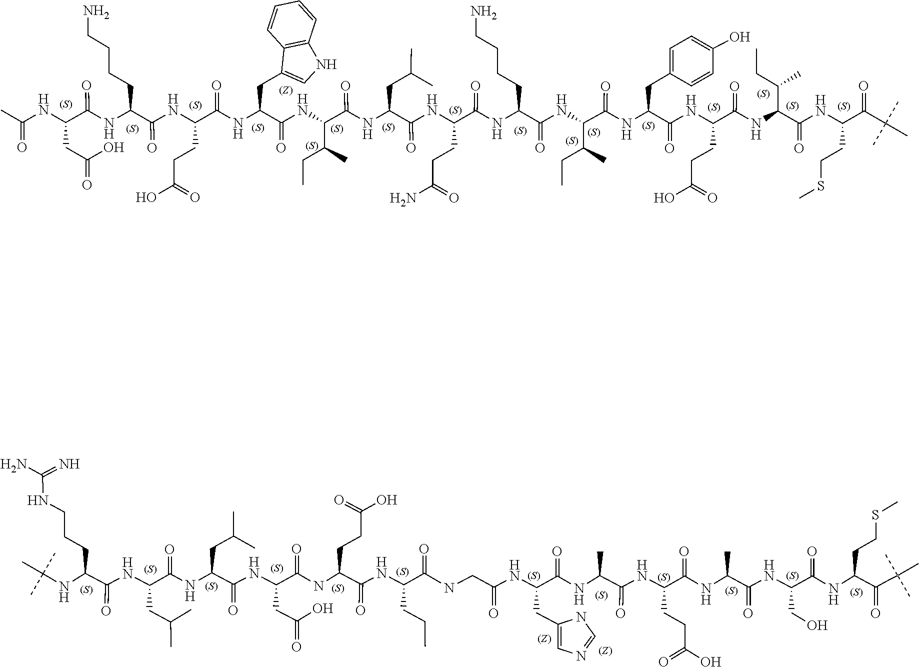

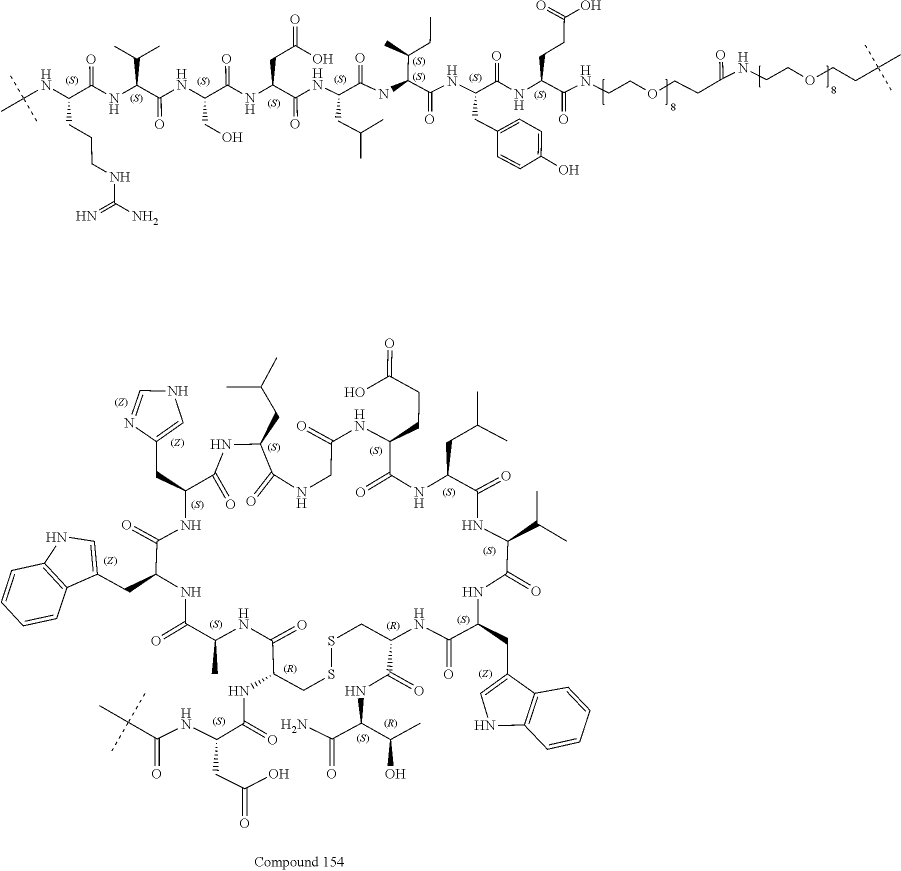

0.20 mmol resin afforded Compound 155 (61.9 mg, 94.0% purity, 4.7% total yield) as a white solid. LCMS: RT=1.06 min, MS cal.: 6542.63, mass observed: [M+4H]⁴⁺=1636.32, [M+6H]⁶⁺=1309.56, [M+7H]⁺=1091.34.

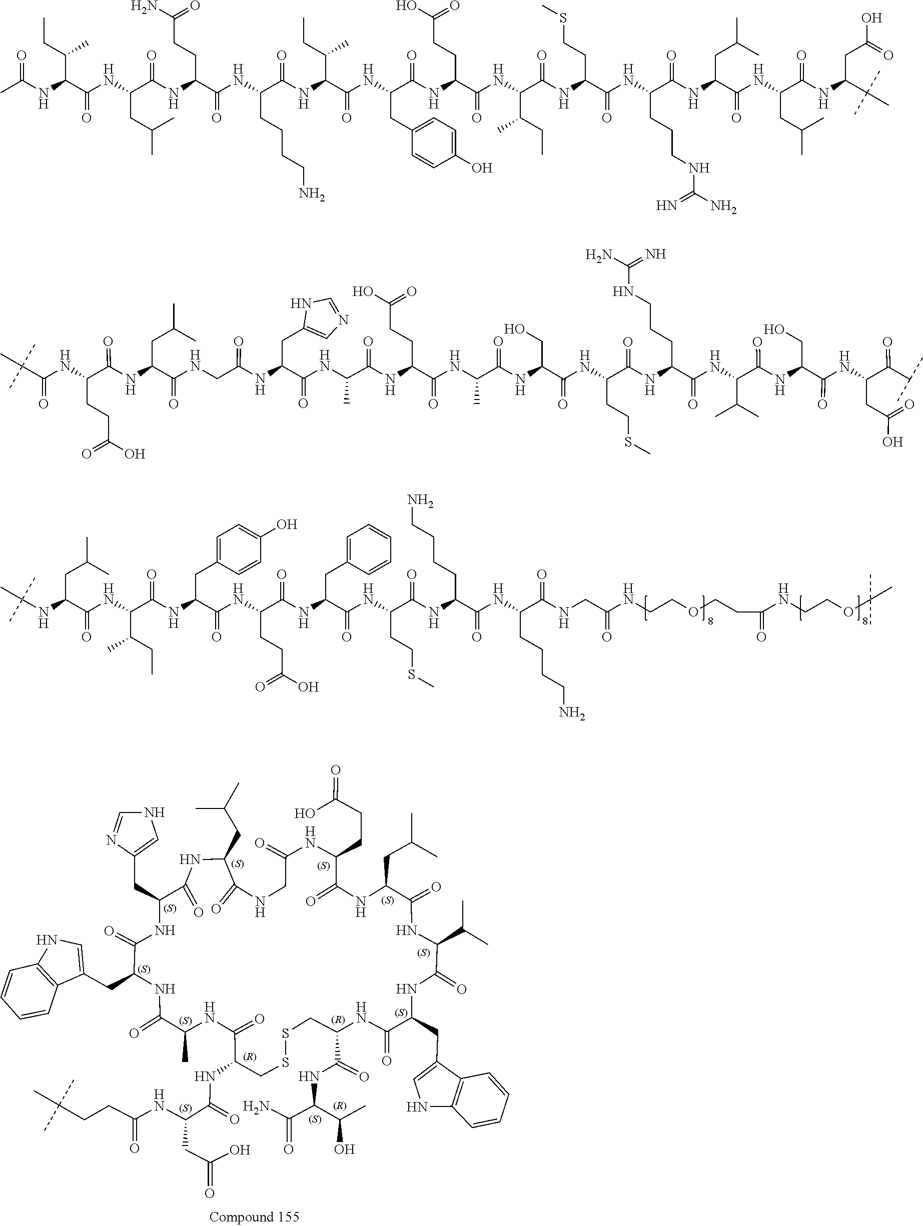

0.20 mmol resin afforded Compound 156 (59.6 mg, 95.7% purity, 4.9% yield in total) as a white solid. LCMS: RT=1.03 min, MS cal.: 6060.02, mass observed: [M+4H]⁴⁺=1516.00, [M+5H]⁵⁺=1218.80, [M+6H]⁶⁺=1010.84.

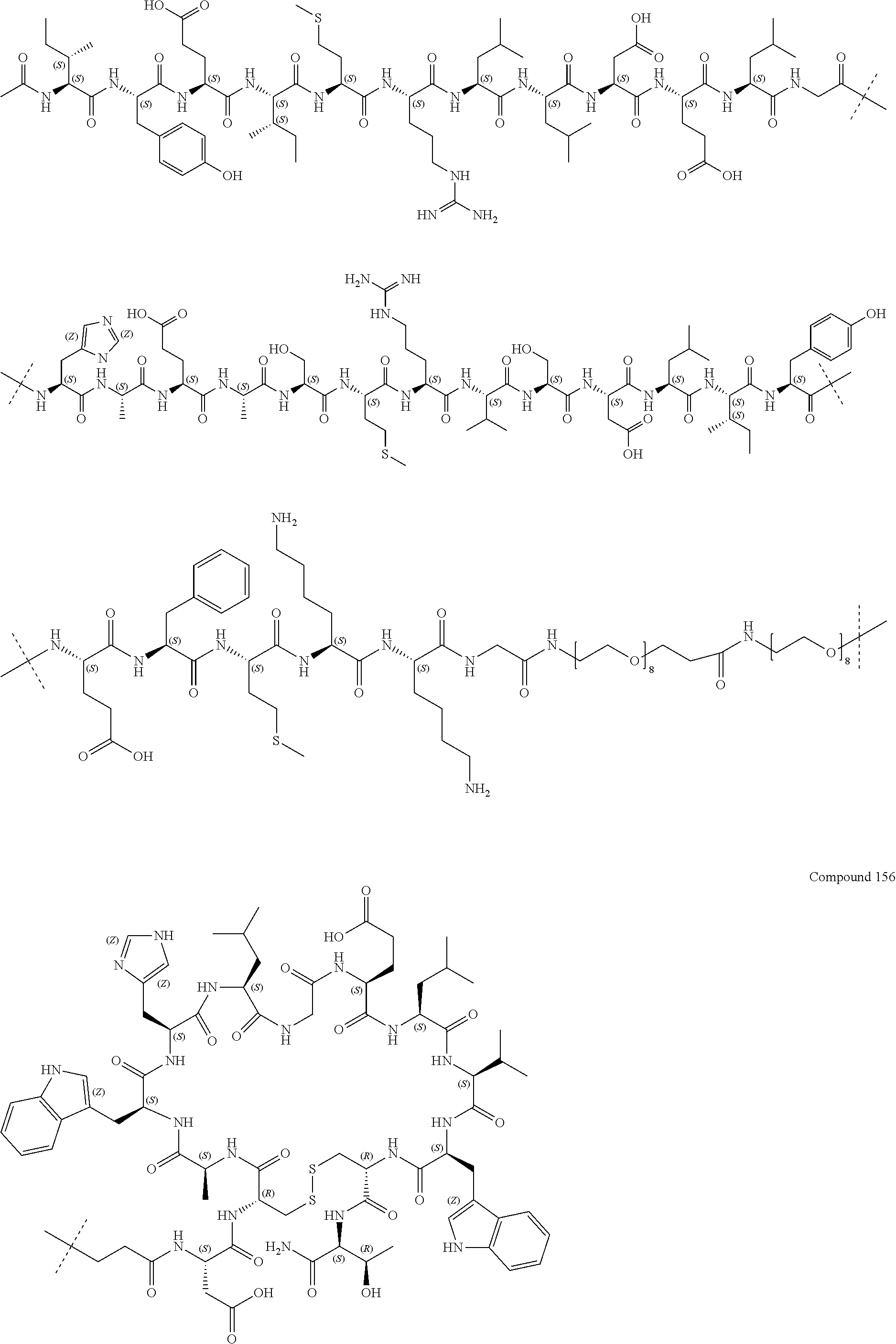

### Example 11. Procedure for Preparation of Compound 157

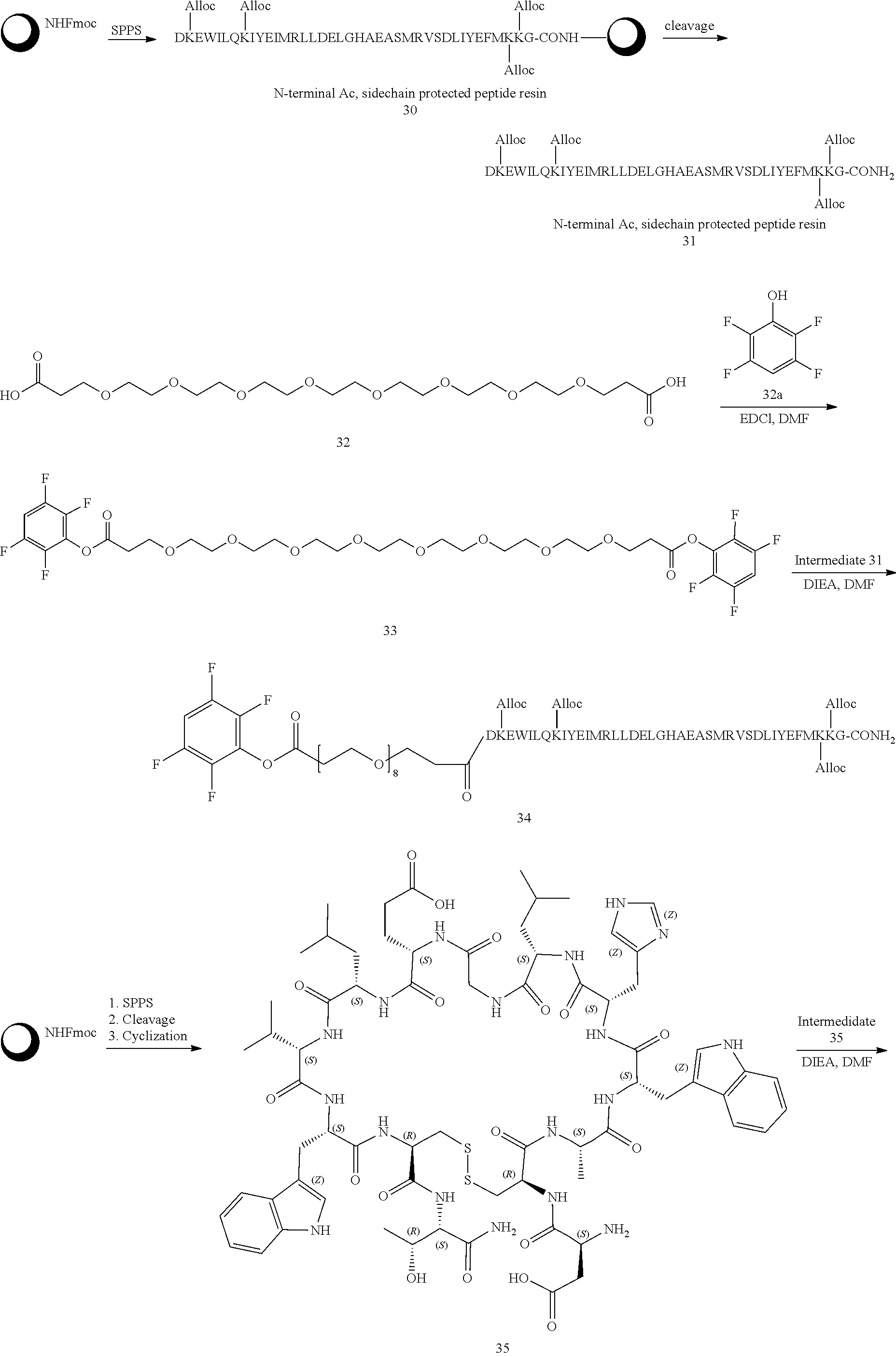

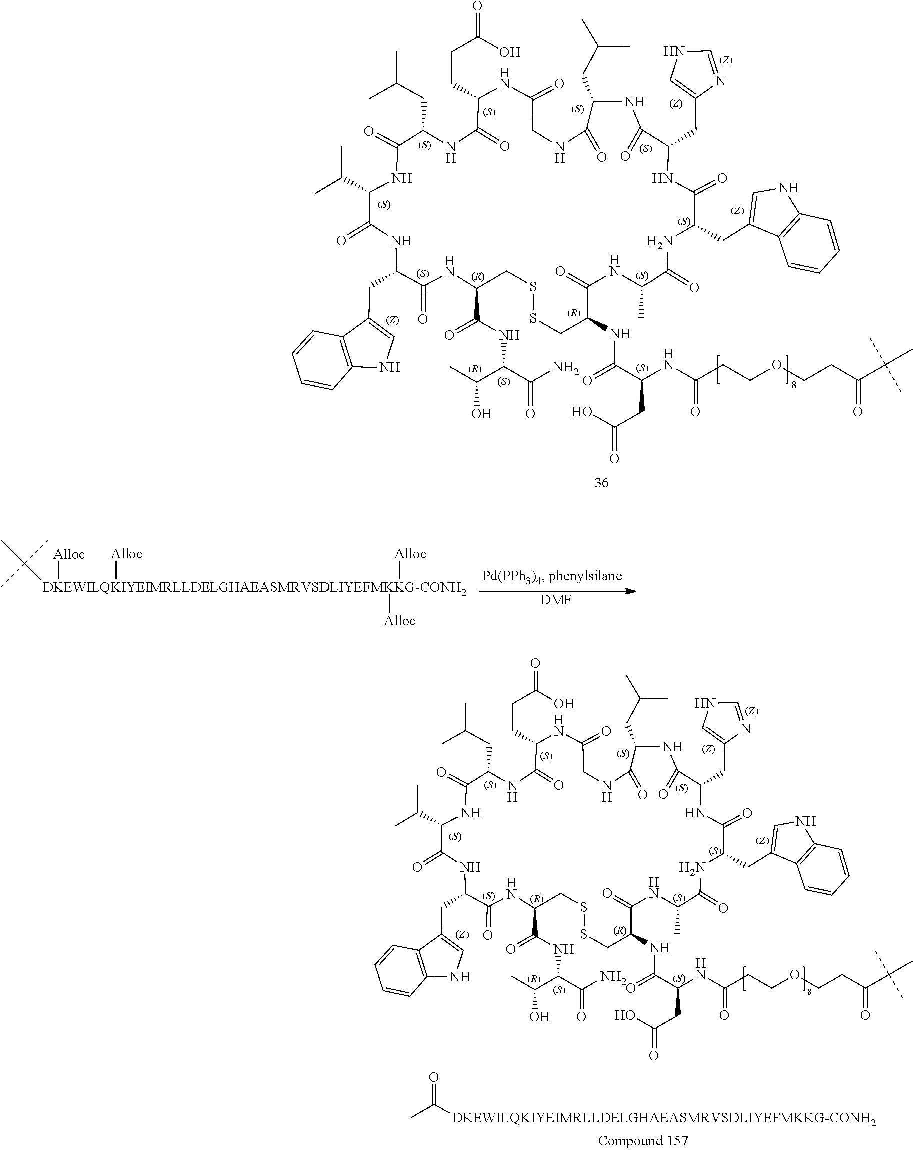

### Preparation of Intermediate 30.

Resin-bound peptide (intermediate 30 (SEQ ID NO:49), Rink amide AM resin, 0.20 mmol) was synthesized using standard Fmoc chemistry.

### Preparation of Intermediate 31 (SEQ ID NO:49).

Peptide cleavage, cyclization and purification.
1) Cleavage solution (TFA/TIS/H₂O, 95/2.5/2.5, v/v/v, 30 mL) was added to the flask containing the side-chain protected resin-bound peptide intermediate 30 (Rink AM resin, 0.20 mmol) at room temperature and stirred for 2 h.
2) After filtration, the filtrate was collected.
3) The filtrate was precipitated with cold isopropyl ether (100 mL). After filtration, the solid was washed with isopropyl ether (50 mL) twice, and the crude peptide was dried under reduced pressure for 2 h to afford intermediate 30 (3.50 g, crude) as a white solid.
4) The residue was purified by prep-HPLC (A: 0.075% TFA/H₂O, B: MeCN) to afford intermediate 31 (310.1 mg, 90% purity, 29.6% yield) as a white solid. LCMS: RT=0.99 min, MS cal.: 5035.77, mass observed: [M+4H]⁴⁺+=1529.7, [M+5H]⁵⁺=1008.0, [M+6H]⁶⁺=840.6.

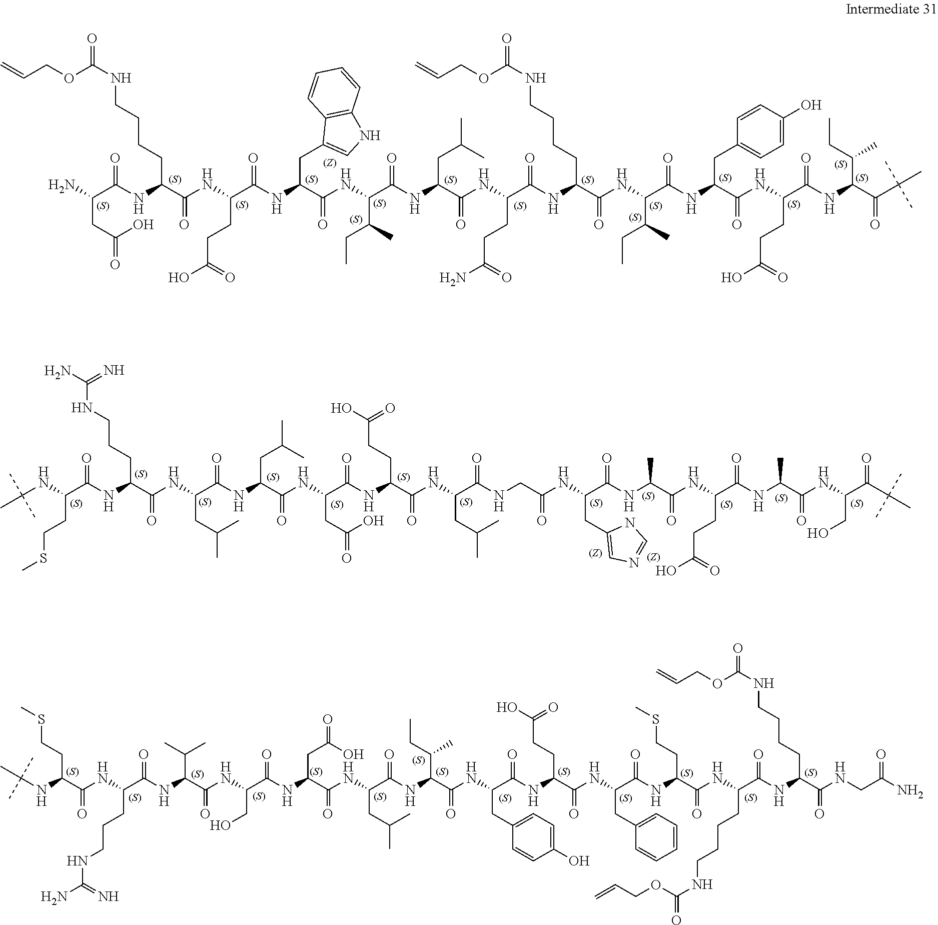

### Preparation of Intermediate 33.

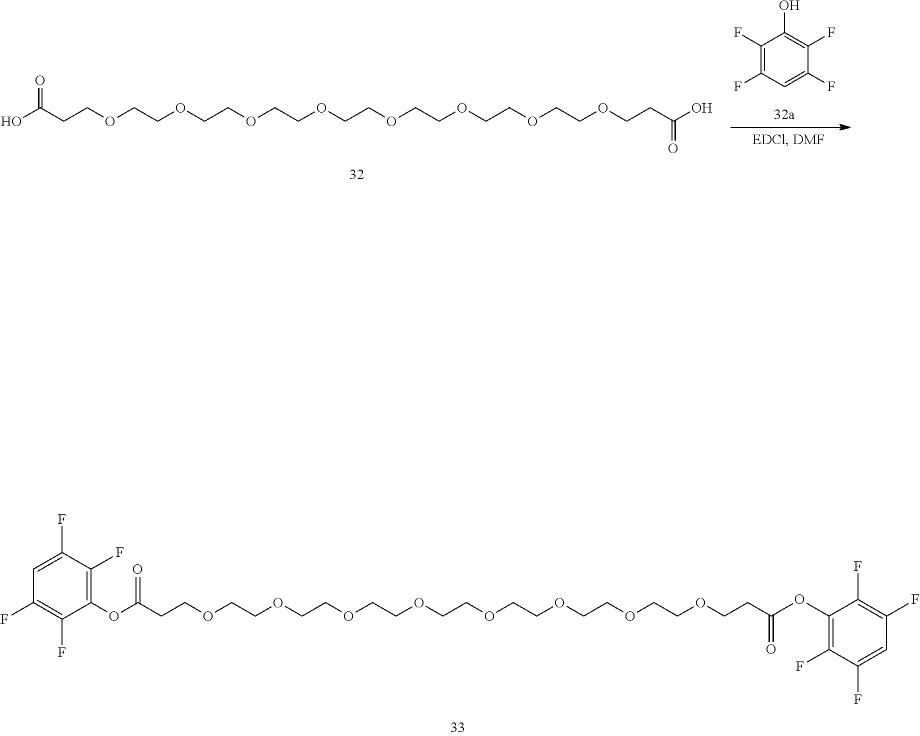 [SMILES: O=C(O)CCOCCOCCOCCOCCOCCOCCOCCOCCC(=O)O ; Oc1c(F)c(F)cc(F)c1F ; O=C(CCOCCOCCOCCOCCOCCOCCOCCOCCC(=O)Oc1c(F)c(F)cc(F)c1F)Oc1c(F)c(F)cc(F)c1F]

To a mixture of intermediate 32 (10.0 g, 21.25 mmol, 1.00 equiv.), EDCI (16.30 g, 85.01 mmol, 4.00 equiv.) in DMF (100 mL) was added 2,3,5,6-tetrafluorophenol (21.18 g, 127.52 mmol, 6.00 equiv.) at 0° C. Then the mixture was stirred at 20° C. for 16 h. The mixture was purified by prep-HPLC (A: 0.075% TFA/H₂O, B: MeCN) directly to afford intermediate 33 (14.0 g, 95% purity, 81.63% yield) as colorless oil. LCMS: RT=1.29 min, MS cal.: 766.62, mass observed: [M+H]⁺=767.5, [M+H₂O+H]+=785.5.

### Preparation of Intermediate 34 (SEQ ID NO:50).

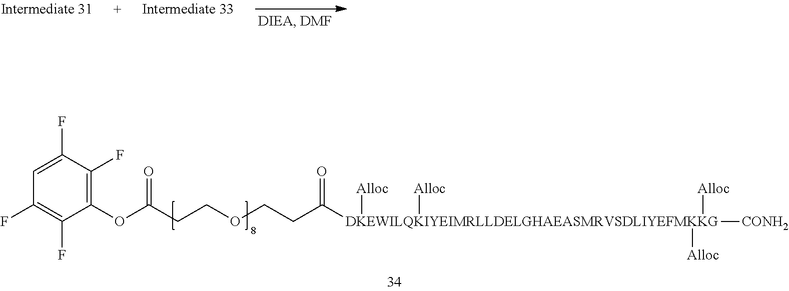

To a mixture of intermediate 33 (228.3 mg, 297.8 mol, 5.00 equiv.) and intermediate 31 (300 mg, 59.6 mol, 1.00 equiv.) in DMF (5 mL) was added DIEA (77.0 mg, 595.7 mol, 10.0 equiv.) at 0° C. The mixture was stirred at 20° C. for 2 h. Monitored by LCMS until intermediate 31 was consumed. The mixture was acidified by 1 M HCl to pH=5, purified by prep-HPLC (A: 0.075% TFA/H₂O, B: MeCN) directly, then lyophilized to afford intermediate 34 (SEQ ID NO:50) (120 mg, 91% purity, 32.5% yield) as a white solid. TOF: RT=1.38 min, MS cal.: 5636.32, mass observed: [M+3H]³⁺=1879.31, [M+4H]⁴⁺=1409.66. [M+5H]⁵⁺=127.82. [M+6H]⁶⁺=912.59.

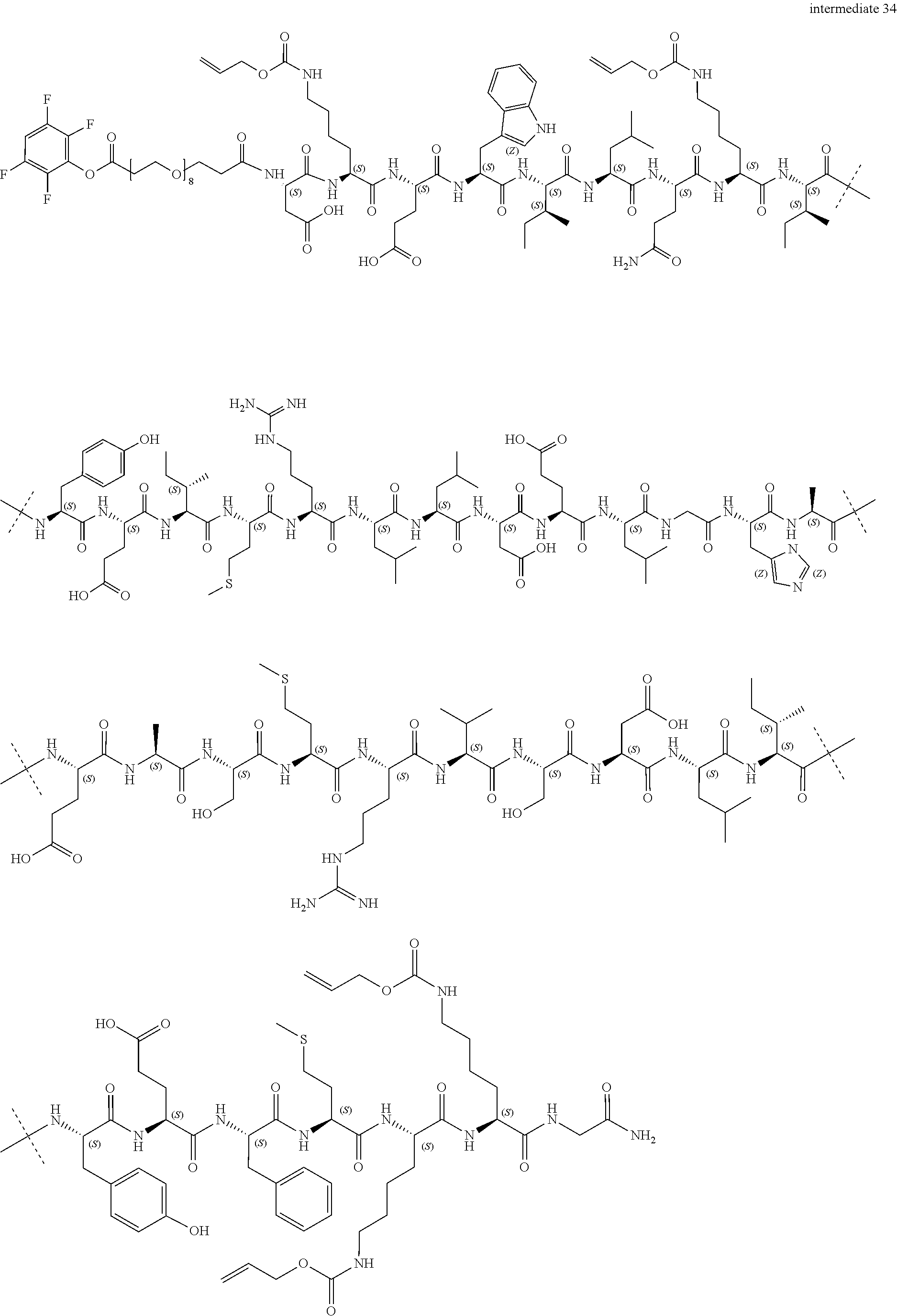

Preparation of intermediate 35 (disulfide cyclic peptide).

Intermediate 35 (disulfide cyclic peptide) was synthesized using the same method as intermediate 19, which was performed by following the procedure mentioned in [[0310)-[0315]. 2.00 mmol rink amide AM resin afforded intermediate 35 (688.0 mg, 85.8% purity, 22.5% yield) as a white solid. LCMS: RT=1.60 min, MS cal.: 1529.74, mass observed: [M+H]⁺=1530.6, [M+2H]²⁺=765.9.

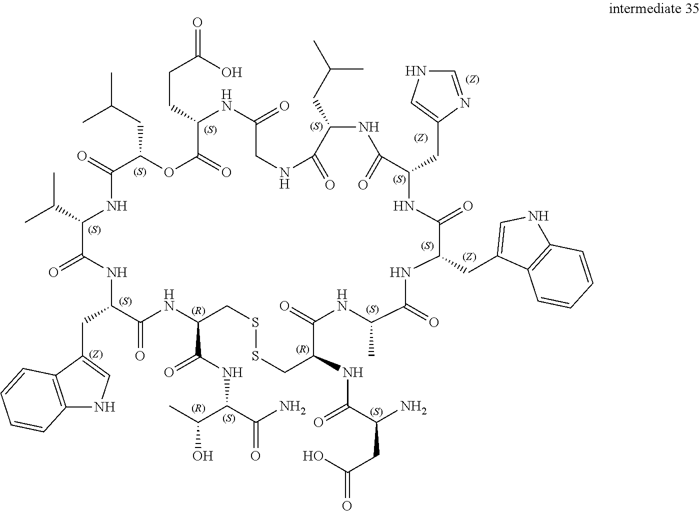

### Preparation of Intermediate 36.

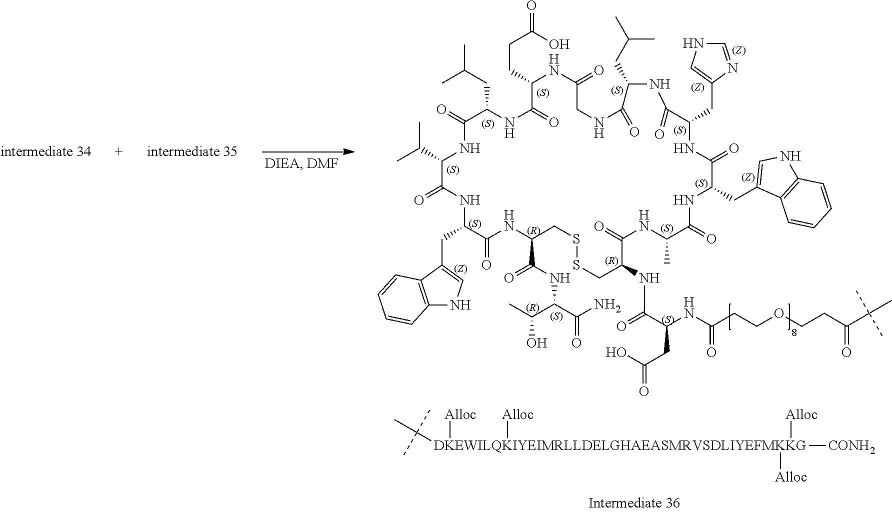

To a mixture of intermediate 35 (32.5 mg, 21.3 μmol, 1.00 equiv.) and intermediate 34 (120 mg, 21.3 mol, 1.00 equiv.) in DMF (1 mL) was added DIEA (2.0 mg, 213.0 mol, 10.0 equiv.) at 0° C. The mixture was stirred at 20° C. for 2 h. Monitored by LCMS until intermediate 34 was consumed. The mixture was acidified by 1 M HCl to pH=5, purified by prep-HPLC (A: 0.075% TFA/H₂O, B: MeCN) directly, then lyophilized to afford intermediate 36 (SEQ ID NO:51) (85.0 mg, 95.9% purity, 54.7% yield) as a white solid. TOF: RT=1.38 min, MS cal.: 6999.98, mass observed: [M+4H]⁴⁺+=1750.24. [M+5H]⁵⁺=1400.39. [M+6H]⁶⁺=1167.16.

### Preparation of Compound 157.

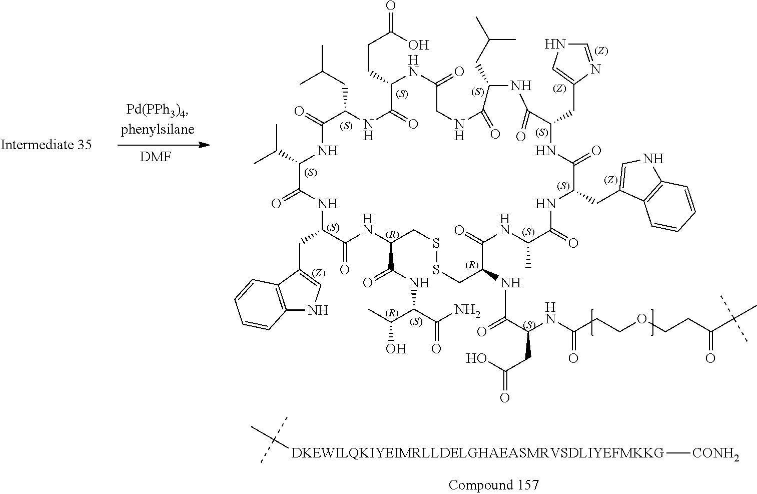

Intermediate 36 (SEQ ID NO:51) (85.0 mg, 12.1 μmol, 1.00 equiv.), phenylsilane (10.8 mg, 242 mol, 12.0 uL, 20.00 equiv.), Pd(PPh₃)₄ (6.98 mg, 6.05 μmol, 0.50 equiv.) in DMF (1 mL) was stirred under N₂ atmosphere at 20° C. for 0.5 h. The mixture was purified by prep-HPLC (A: 0.075% TFA/H₂O, B: MeCN) directly, then lyophilized to afford Compound 157 (SEQ ID NO:52) (27.9 mg, 97.6% purity, 32.9% yield) as a white solid. LCMS: RT=1.02 min, MS cal.: 6663.69, mass observed: [M+4H]⁴⁺+=1666.50, [M+5H]⁵⁺=1333.50, [M+6H]⁶⁺=1111.24, [M+8H]⁸⁺=833.90, [M+9H]⁹⁺=740.89.

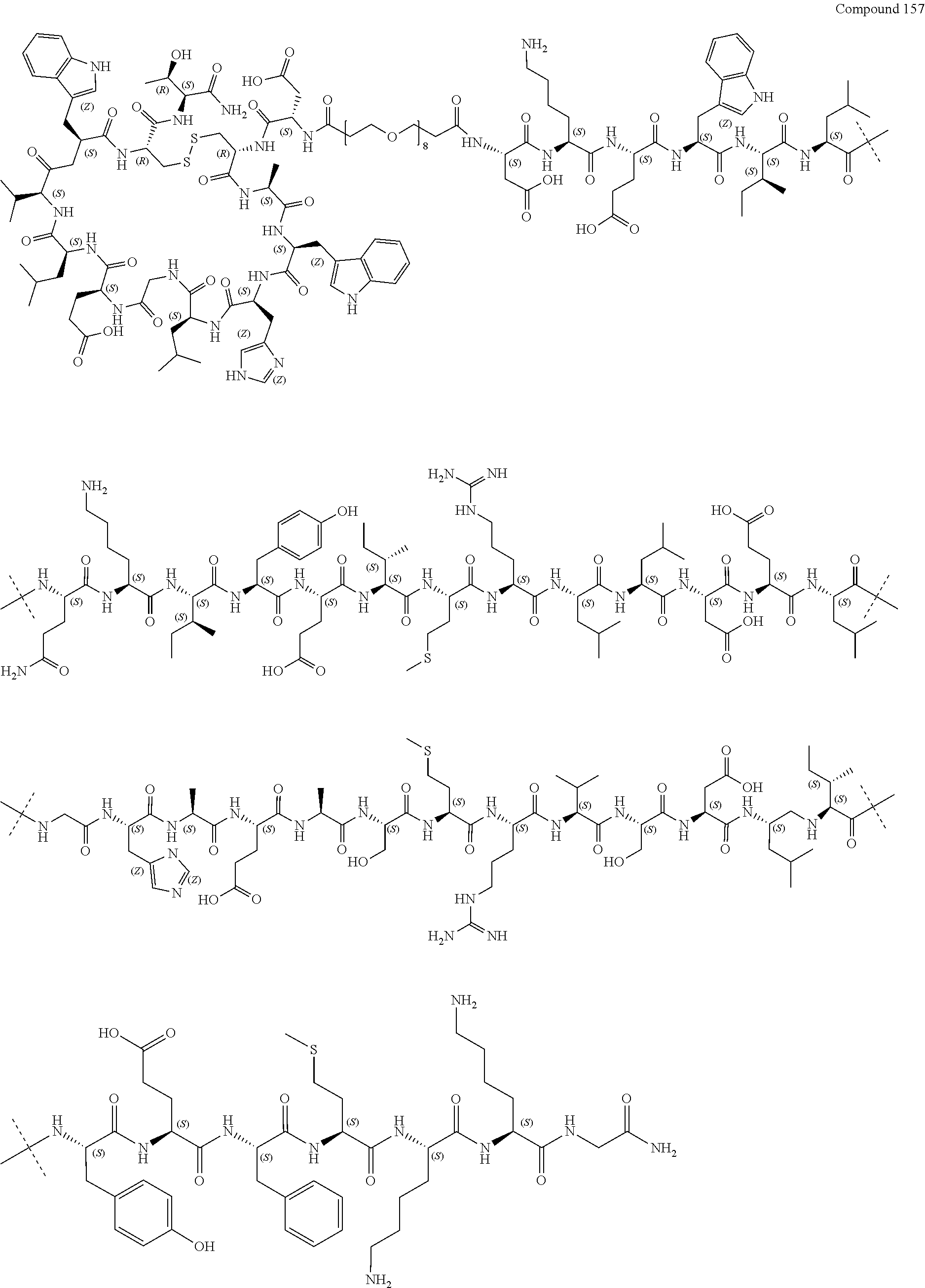

Compound 128, Compound 158 were synthesized using the same method as Compound 157.

0.20 mmol resin afforded Compound 158 (5.5 mg, 78.2% purity, 0.3% total yield) as a white solid. LCMS: RT=1.13 min, MS cal.: 9197.47, mass observed: [M+4H]⁴⁺+=1840.12, [M+5H]⁵⁺=1553.65, [M+6H]⁶⁺=1314.16.

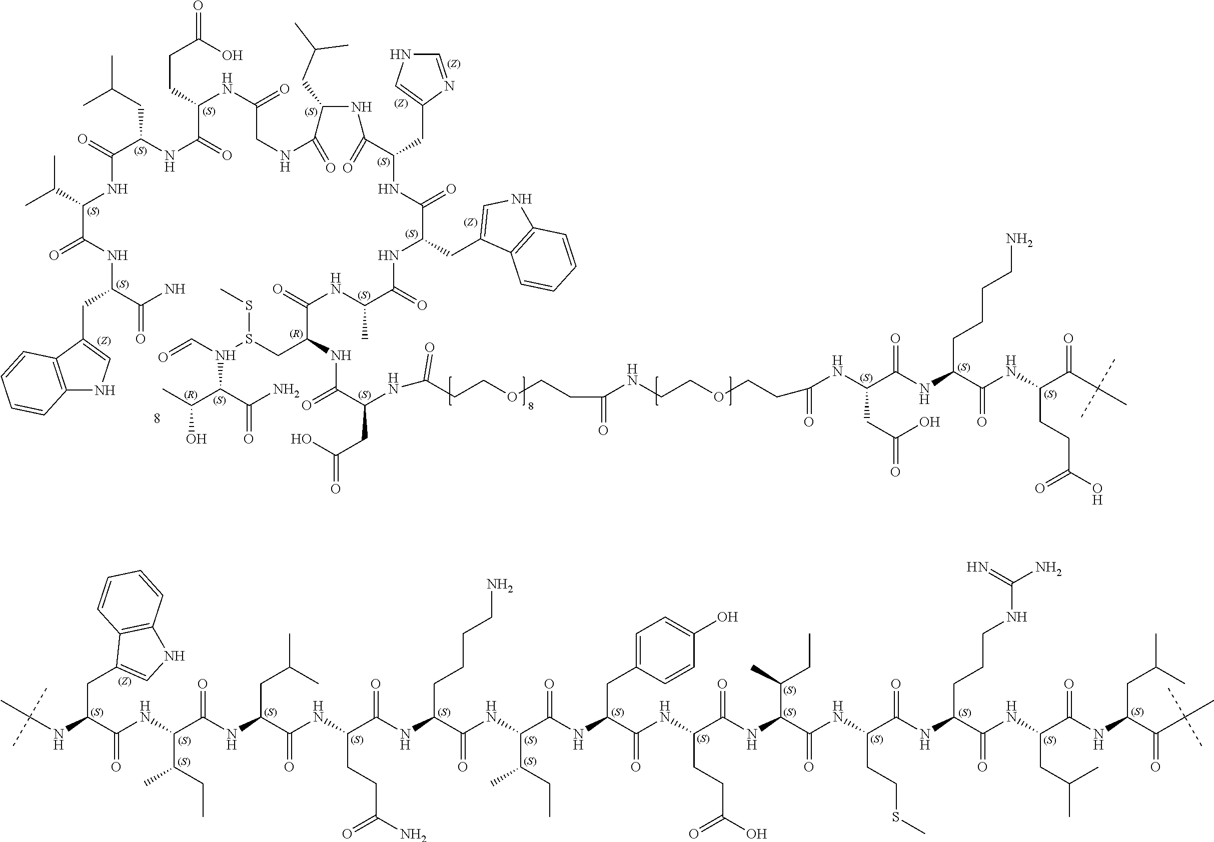

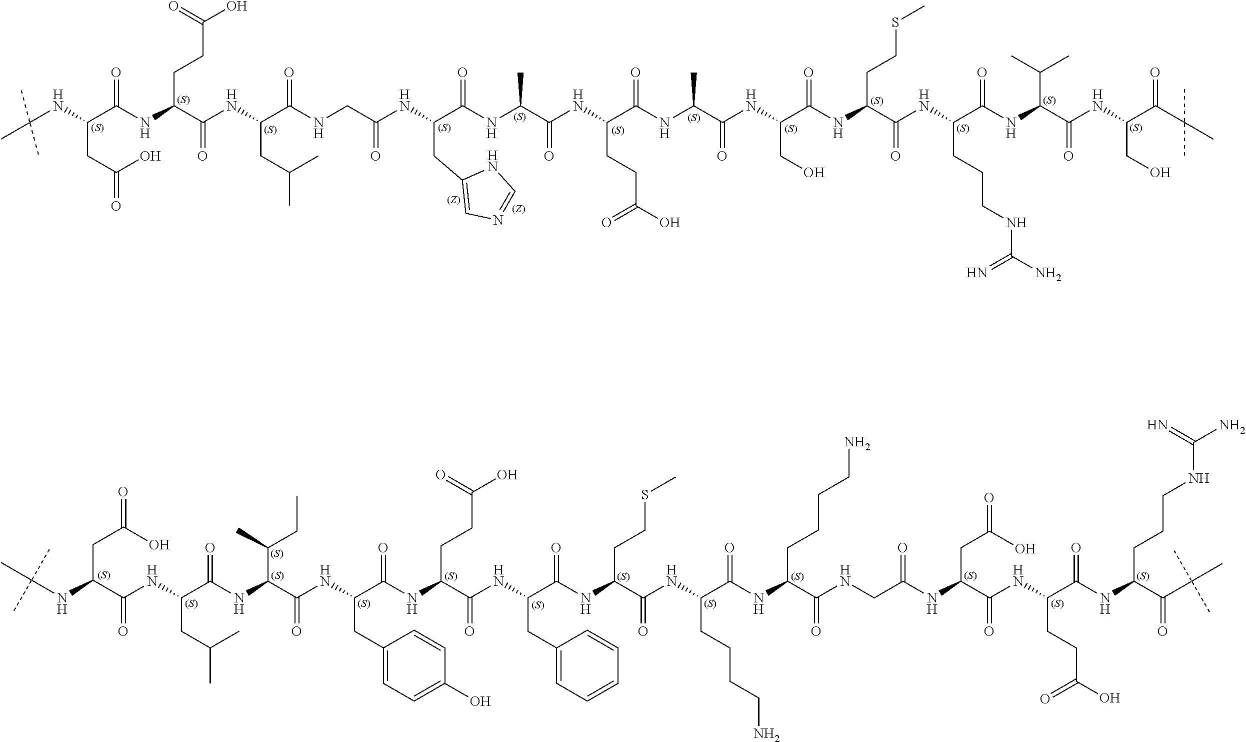

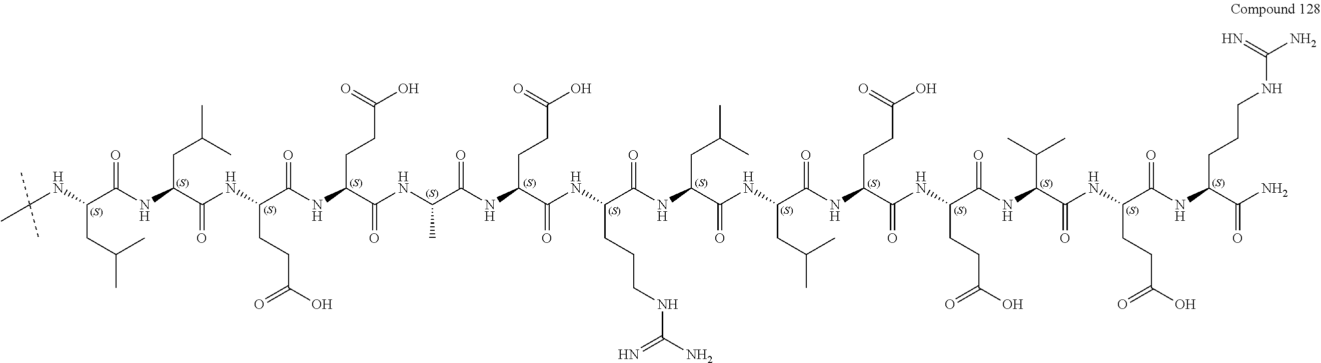

0.20 mmol resin afforded Compound 158 (11.7 mg, 97.1% purity, 4.9% total yield) as a white solid. LCMS: RT=1.15 min, MS cal.: 9200.49, mass observed: [M+5H]⁵⁺=1841.24, [M+7H]⁷⁺=1315.75, [M+8H]⁸⁺=1151.11.

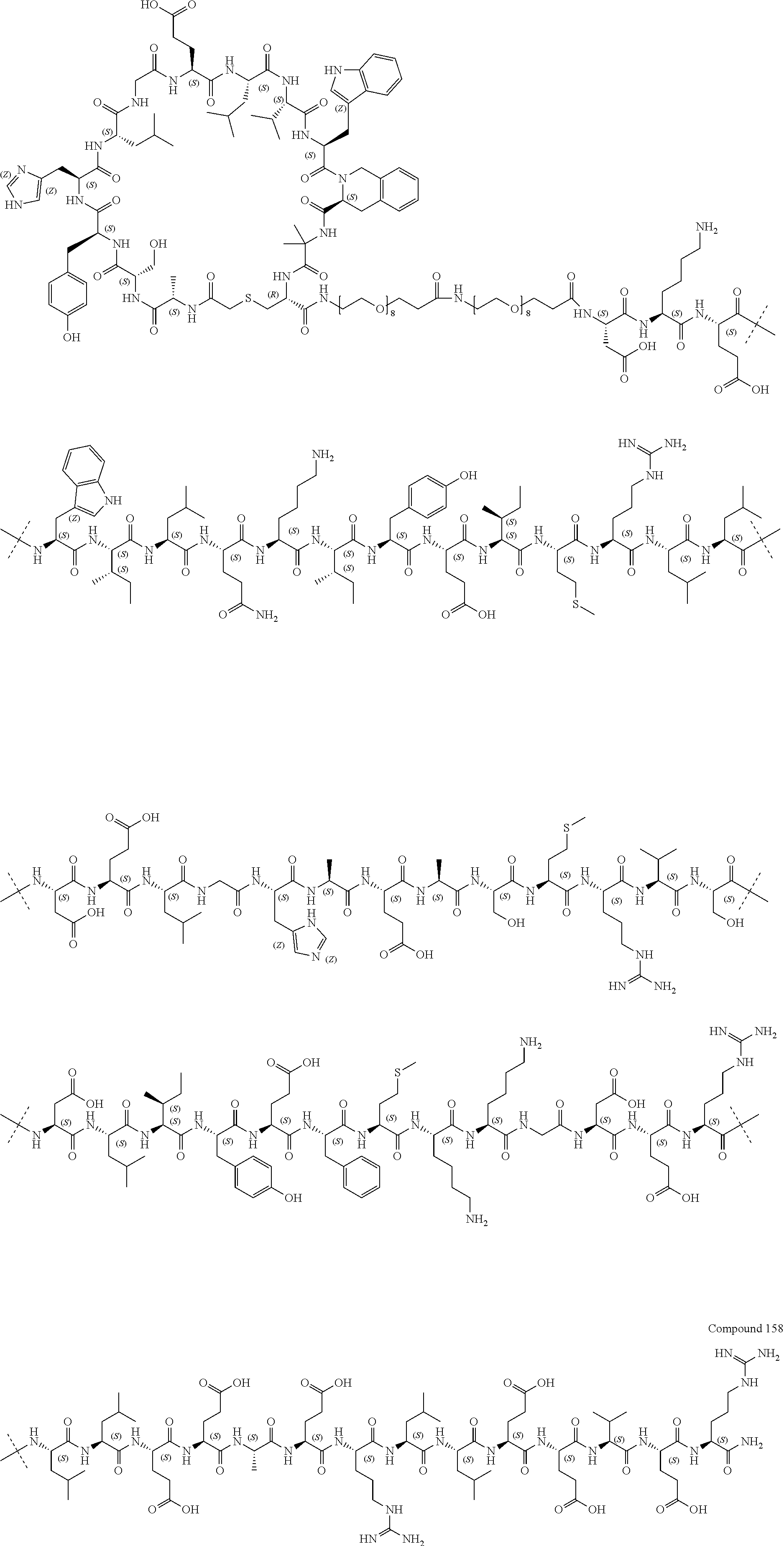

### Example 12. Procedure for Preparation of Compound 159

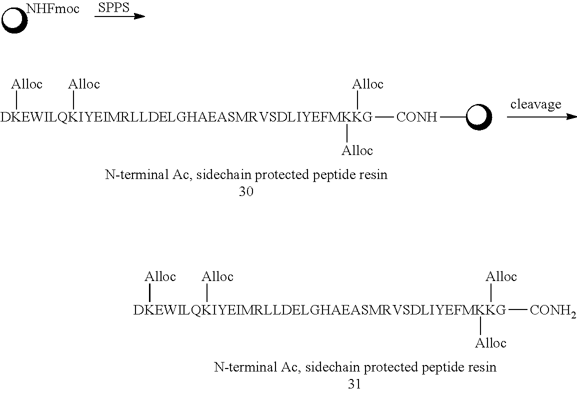

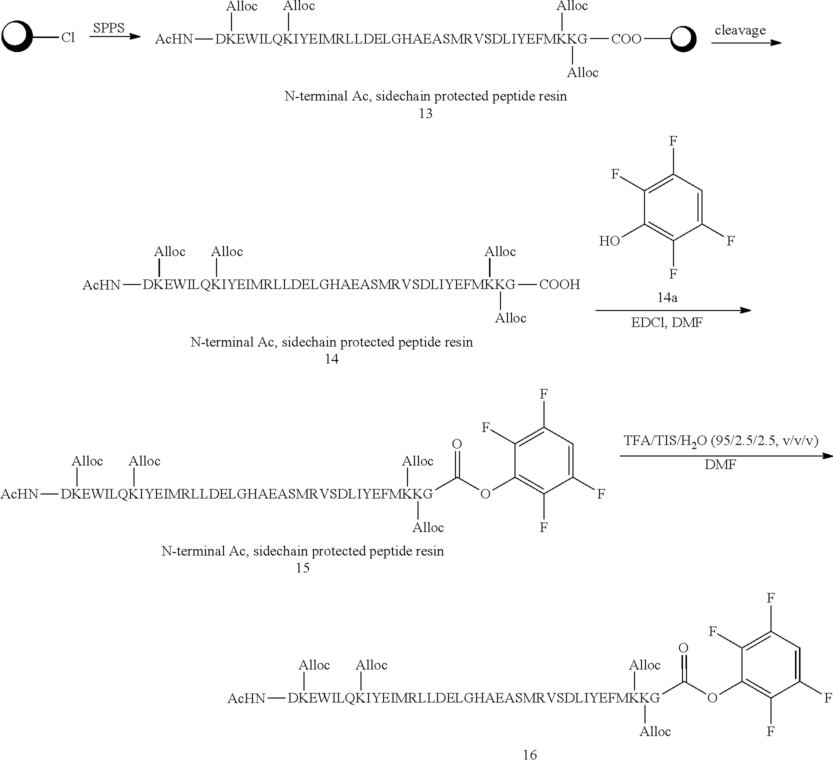

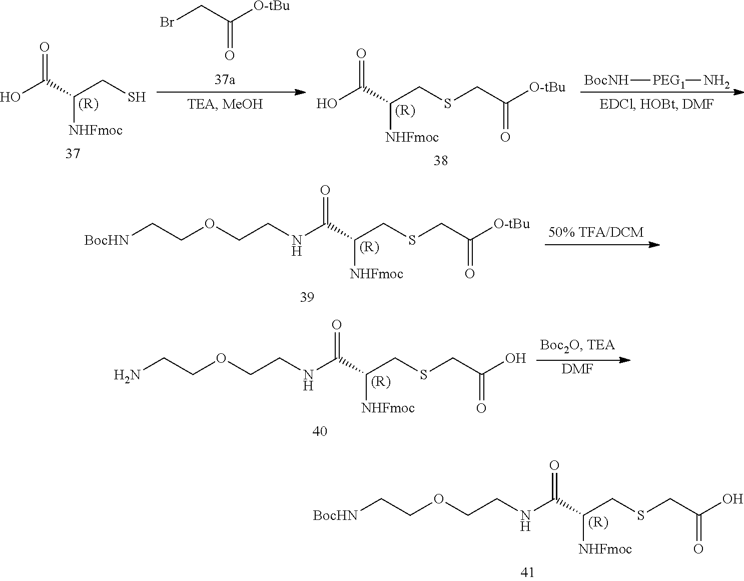

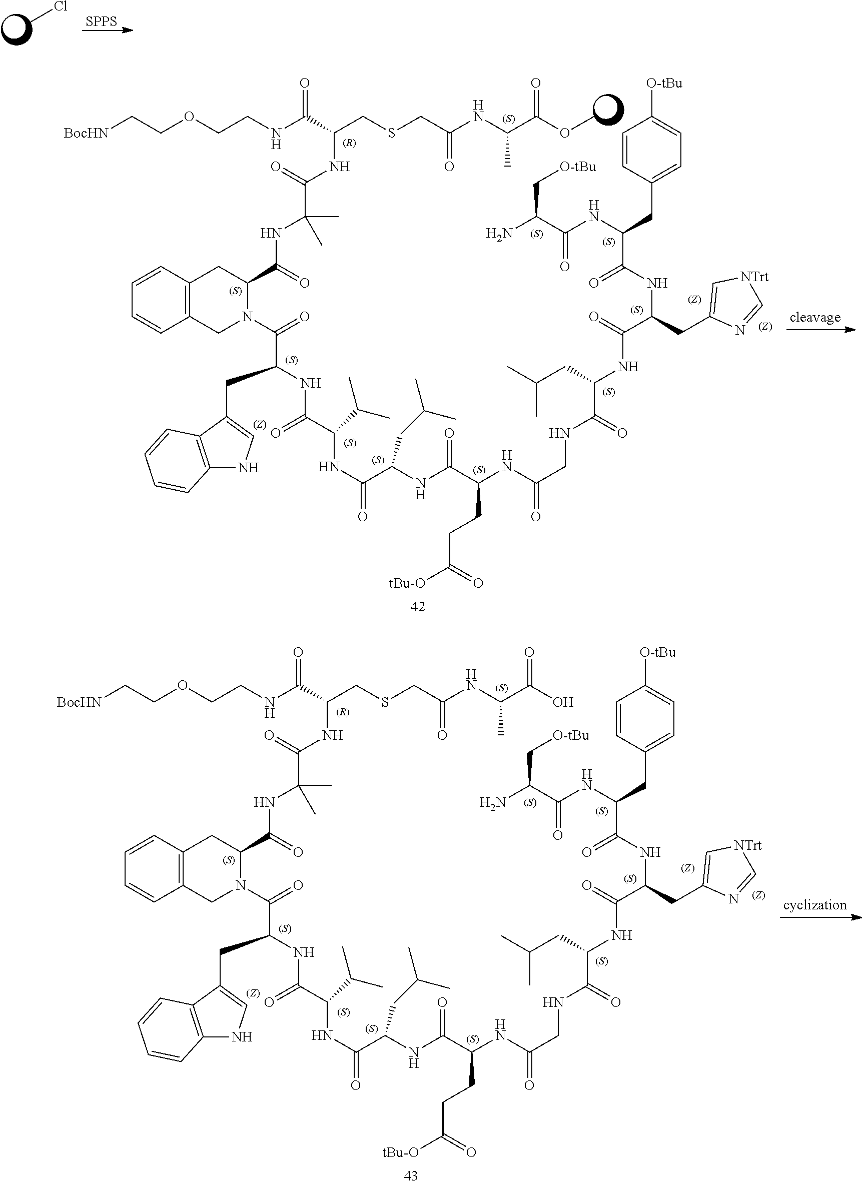

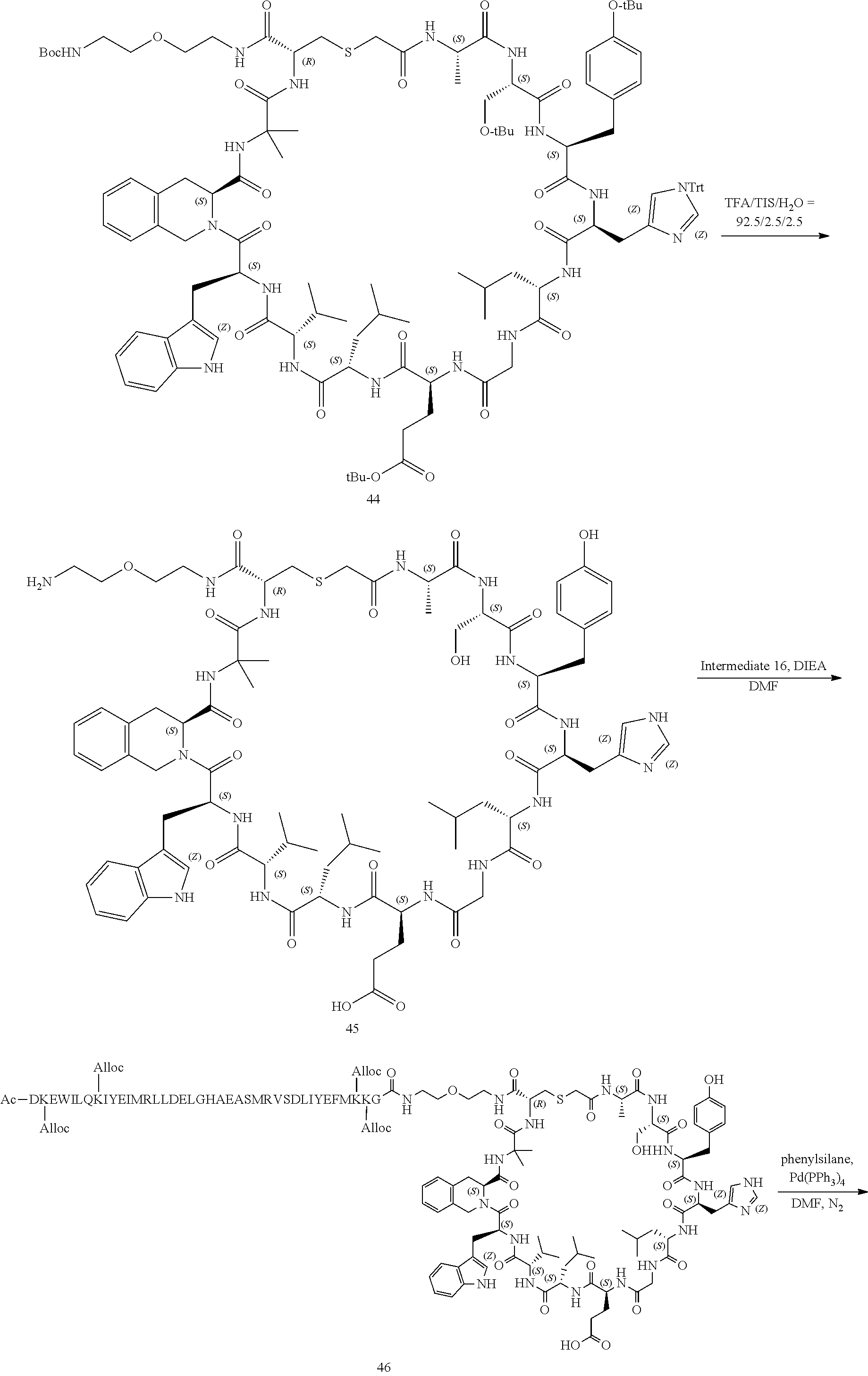

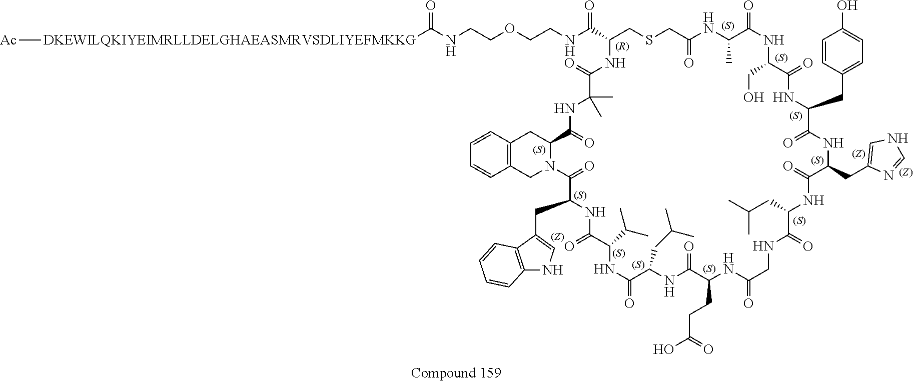

Intermediate 16 was synthesized using standard Fmoc chemistry, which was performed by following the procedure mentioned in Example 10.

### Preparation of Intermediate 38.

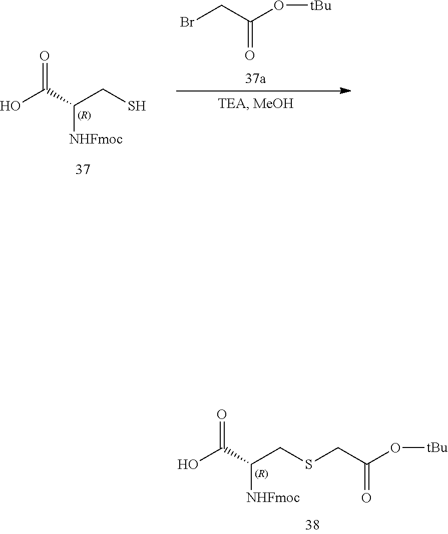

To a mixture of intermediate 37 (10.0 g, 29.12 mmol, 1.00 equiv.), DIEA (7.53 g, 58.24 mmol, 10.14 mL, 2.00 equiv.) in MeOH (300 mL) was added a solution of 37a (5.68 g, 29.12 mmol, 4.30 mL, 1.00 equiv.) in MeOH (50 mL) dropwise at 20° C. under N₂. Then the solution was stirred at 20° C. for 3 h. The mixture was acidified by 1 M HCl to pH=6. Then the solvent was removed under reduced pressure and the residue was diluted with DCM (500 mL), washed with H₂O (200 mL), brine (200 mL), dried over anhydrous Na₂SO₄, and concentrated under reduced pressure to get intermediate 38 (11.4 g, crude) as colorless oil.

### Preparation of Intermediate 39.

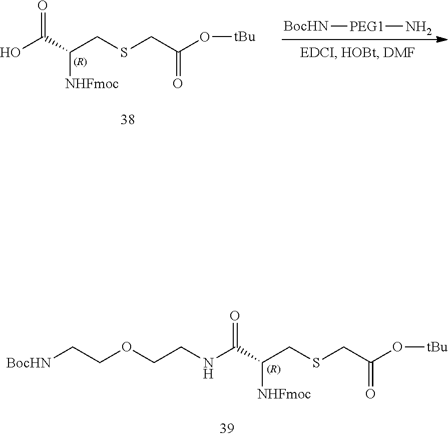

To a solution of intermediate 38 (3.00 g, 6.56 mmol, 1.00 equiv.) and BocHN-PEG1-NH₂ (1.34 g, 6.56 mmol, 1.00 equiv.) in DMF (30 mL) was added EDCI (2.51 g, 13.11 mmol, 2.00 equiv.) and HOBt (1.33 g, 9.84 mmol, 1.50 equiv.) at 0° C. Then the reaction mixture was stirred at 20° C. for 2 h. The DMF was removed under reduced pressure. The residue was diluted with DCM (200 mL), washed with 0.5 M HCl (50 mL), H₂O (50 mL), brine (50 mL), dried over anhydrous Na₂SO₄, and concentrated under reduced pressure to afford intermediate 39 (2.80 g, crude) as a white solid.

### Preparation of Intermediate 40.

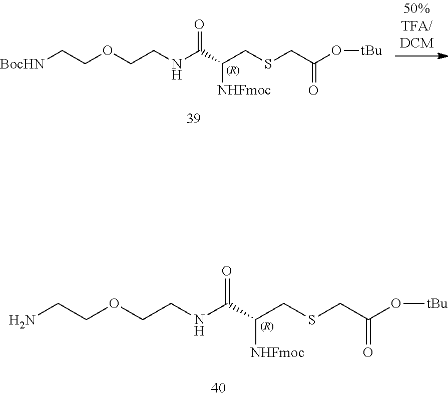

A mixture of intermediate 39 (2.8 g, 4.35 mmol) in TFA/DMF (1/1, v/v, 30 mL) was stirred at 20° C. for 1 h. Then the solvent was removed under reduced pressure. The residue was purified by prep-HPLC (C18, A: 0.075 TFA/H₂O, B: MeCN), lyophilized to afford intermediate 40 (1.80 g, 3.69 mmol, 84.8% yield) as colorless oil.

### Preparation of Intermediate 41.

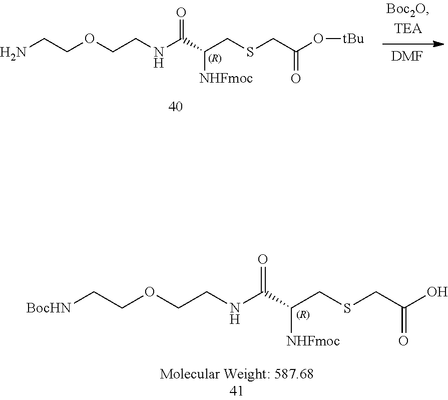

To a solution of intermediate 40 (1.80 g, 3.69 mmol, 1.00 equiv.) in DMF (20 mL) was added Boc₂O (966.8 mg, 4.43 mmol, 1.02 mL, 1.20 equiv.) and TEA (747.1 mg, 7.38 mmol, 1.03 mL, 2.00 equiv.) at 20° C. Then the reaction mixture was stirred at 20° C. for 2 h. The mixture was purified by prep-HPLC (A: 0.075% TFA/H₂O, B: MeCN), lyophilized to afford intermediate 41 (0.90 g, 86.7% purity, 41.48% yield) as white solid. LCMS: RT=0.953 min, MS cal.: 587.68, mass observed: [M+H]⁺=588.1, [M-Boc+H]⁺=488.1

### Preparation of Intermediate 45.

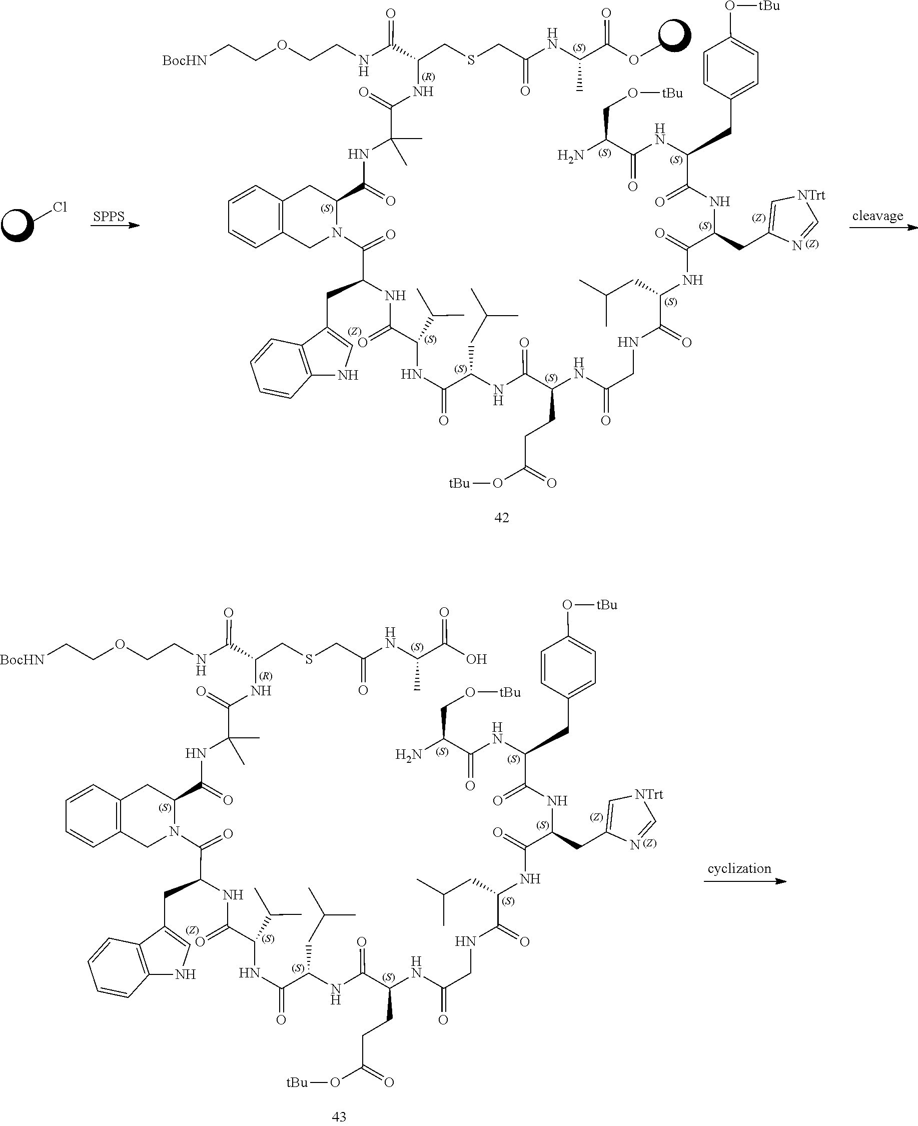

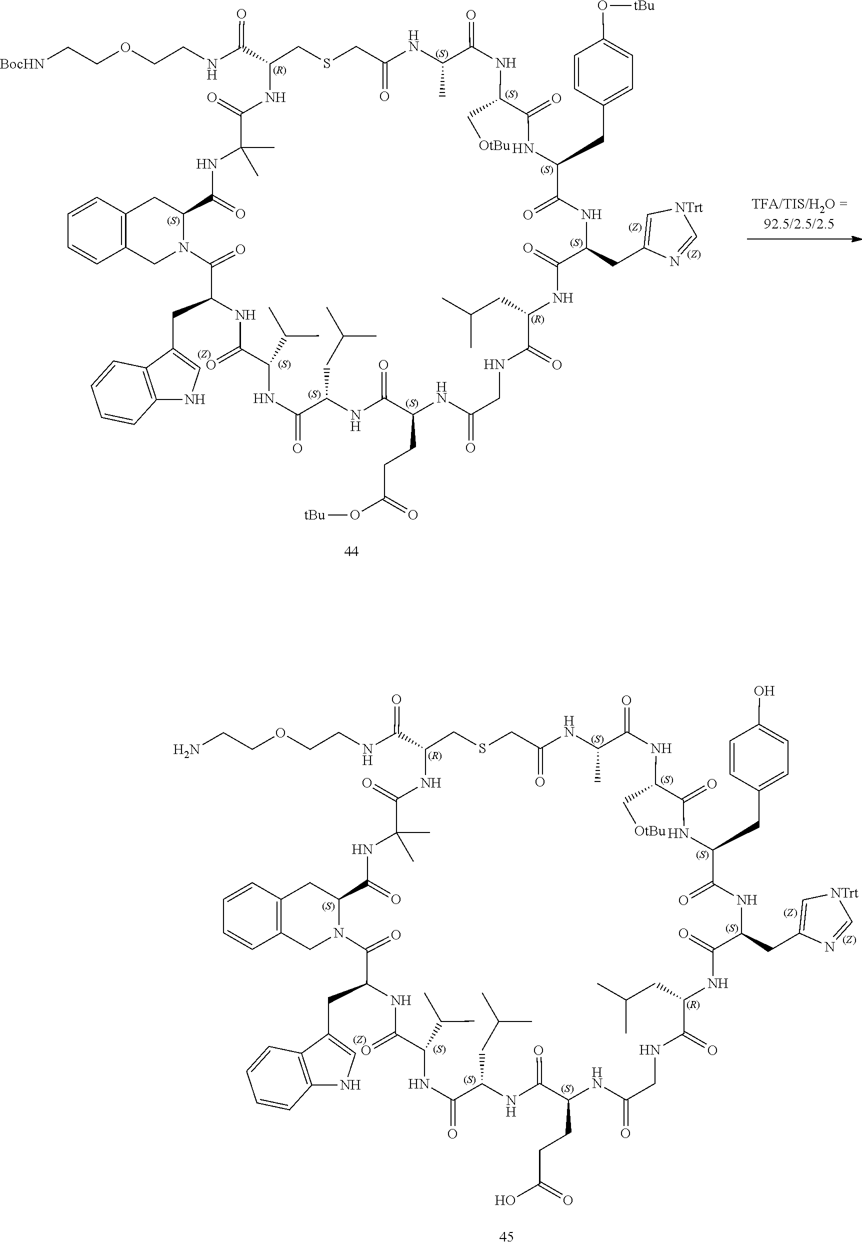

Peptide was synthesized using standard Fmoc chemistry (CTC resin).

Resin preparation: To the vessel containing CTC Resin (1.50 g, 1.50 mmol, 1.50 mmol/g) and intermediate 41 (880.5 mg, 1.50 mmol, 1.00 equiv.) in DCM (15 mL) was added DIEA (4.00 equiv.) dropwise and mixed for 2 h with N₂ bubbling at 20° C. Then MeOH (1.5 mL) was added and bubbled with N₂ for another 30 mN. The resin was washed with DMF (15 mL)*5, followed by the addition of 20% (piperidine in DMF (30 mL) and bubbled with N₂ for 30 min at 20° C. for Fmoc deprotection. The mixture was filtered and the resin was washed with DMF (30 mL)*5 before proceeding to next step.

Coupling: A solution of Fmoc-Aib-OH (1.46 g, 3.00 equiv.), HBTU (1.62 g, 2.85 equiv.) in DMF (15 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (30 mL)*5.

Deprotection: 20% piperidine in DMF (30 mL) was added to the resin and the mixture was bubbled with N₂ for 30 min at 20° C. The deprotection reaction was monitored by ninhydrin test, if it showed blue or brownish red, the reaction was completed. The resin was then washed with DMF (30 mL) *5.

The coupling and deprotection steps were repeated for the following amino acids elongation: Number #3-12, Table 10.

TABLE 10
The list of amino acids and the corresponding reagents used on SPPS.

| # | Materials | Coupling reagents |
|---|---|---|
| 1 | Intermediate 41 (1.00 equiv.) | DIEA (4.00 equiv.) |
| 2 | Fmoc-Aib-OH (3.00 equiv.) | HBTU (2.85 equiv.) and DIEA (6.00 equiv.) |
| 3 | Fmoc-Tic-OH (3.00 equiv.) | HBTU (2.85 equiv.) and DIEA (6.00 equiv.) |
| 4 | Fmoc-Trp-OH (3.00 equiv.) | HBTU (2.85 equiv.) and DIEA (6.00 equiv.) |
| 5 | Fmoc-Val-OH (3.00 equiv.) | HBTU (2.85 equiv.) and DIEA (6.00 equiv.) |
| 6 | Fmoc-Leu-OH (3.00 equiv.) | HBTU (2.85 equiv.) and DIEA (6.00 equiv.) |
| 7 | Fmoc-Glu(OtBu)-OH (3.00 equiv.) | HBTU (2.85 equiv.) and DIEA (6.00 equiv.) |
| 8 | Fmoc-Gly-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-His(Trt)-OH (3.00 equiv.) | HBTU (2.85 equiv.) and DIEA (6.00 equiv.) |
| 11 | Fmoc-Tyr(+Bu)-OH (3.00 equiv.) | HBTU (2.85 equiv.) and DIEA (6.00 equiv.) |
| 12 | Fmoc-Ser(+Bu)-OH (3.00 equiv.) | HBTU (2.85 equiv.) and DIEA (6.00 equiv.) |

Peptide cleavage, cyclization, TFA de-protection and purification.

Cleavage solution (HFIP/DCM, 1/5, v/v, 50 mL) was added to the flask containing the side-chain protected resin-bound peptide intermediate 42 (CTC resin, 3.5 g, 1.50 mmol) at room temperature and stirred for 2 h. After filtration, the filtrate was collected. The filtrate was concentrated under reduced pressure. The residue was added MeCN/H₂O (1/1, v/v, 50 mL), lyophilized to afford intermediate 43 (1.30 g, crude) as a white solid. To a mixture of intermediate 43 (1.30 g, 0.60 mmol, 1.00 equiv.) in DCM (600 mL) was added HATU (460.0 mg, 2.00 equiv.) and DIEA (309.6 mg, 4.00 equiv.) at 20° C. Then the mixture was stirred at 20° C. for 1 h. The mixture was washed with 0.5 M HCl (100 mL), brine (100 mL), dried over anhydrous Na₂SO₄, and concentrated under reduced pressure to afford intermediate 44 (1.30 g, crude) as a white solid.

A mixture of intermediate 44 (1.30 g, crude) in TFA/TIS/H₂O (92.5/2.5/2.5, v/v/v, 30 mL) was stirred at 20° C. for 1 h. The mixture was precipitated with cold isopropyl ether (150 mL). After filtration, the solid was washed with isopropyl ether (50 mL) twice, and the crude peptide was dried under reduced pressure. The crude peptide was purified by prep-HPLC (A: 0.075% TFA/H₂O, B: MeCN), followed by lyophilization to afford intermediate 45 (140.0 mg, 80% purity, 6.30% yield) as a white solid. LCMS: RT=0.90 min, MS cal.: 1647.89, mass observed: [M+2H]²⁺=824.55.

Preparation of Intermediate 46 (fragments coupling) (SEQ ID NO:53).

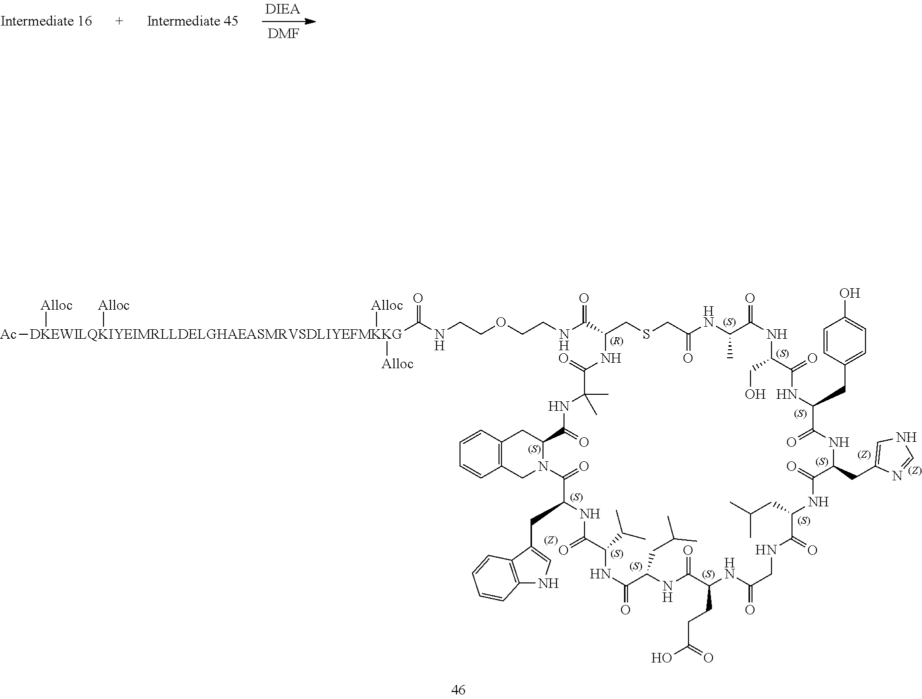

To a mixture of intermediate 16 (185.24 mg, 35.4 mol, 1.00 equiv.) and intermediate 45 58.4 mg, 35.4 μmol, 1.00 equiv.) in DMF (0.5 mL) was added DIEA (45.8 mg, 354 μmol, 10.0 equiv.) at 20° C. The mixture was stirred at 20° C. for 2 h. Monitored by LCMS until intermediate 16 was consumed. The mixture was acidified by 1 M HCl to pH=5, purified by prep-HPLC (A: 0.075% TFA/H₂O, B: MeCN) directly, then lyophilized to afford intermediate 46 (97.0 mg, 80% purity, 32.0% yield) as a white solid. TOF: RT=2.22 min, MS cal.: 6708.67, mass observed: [M+4H]⁴⁺+=1677.82.

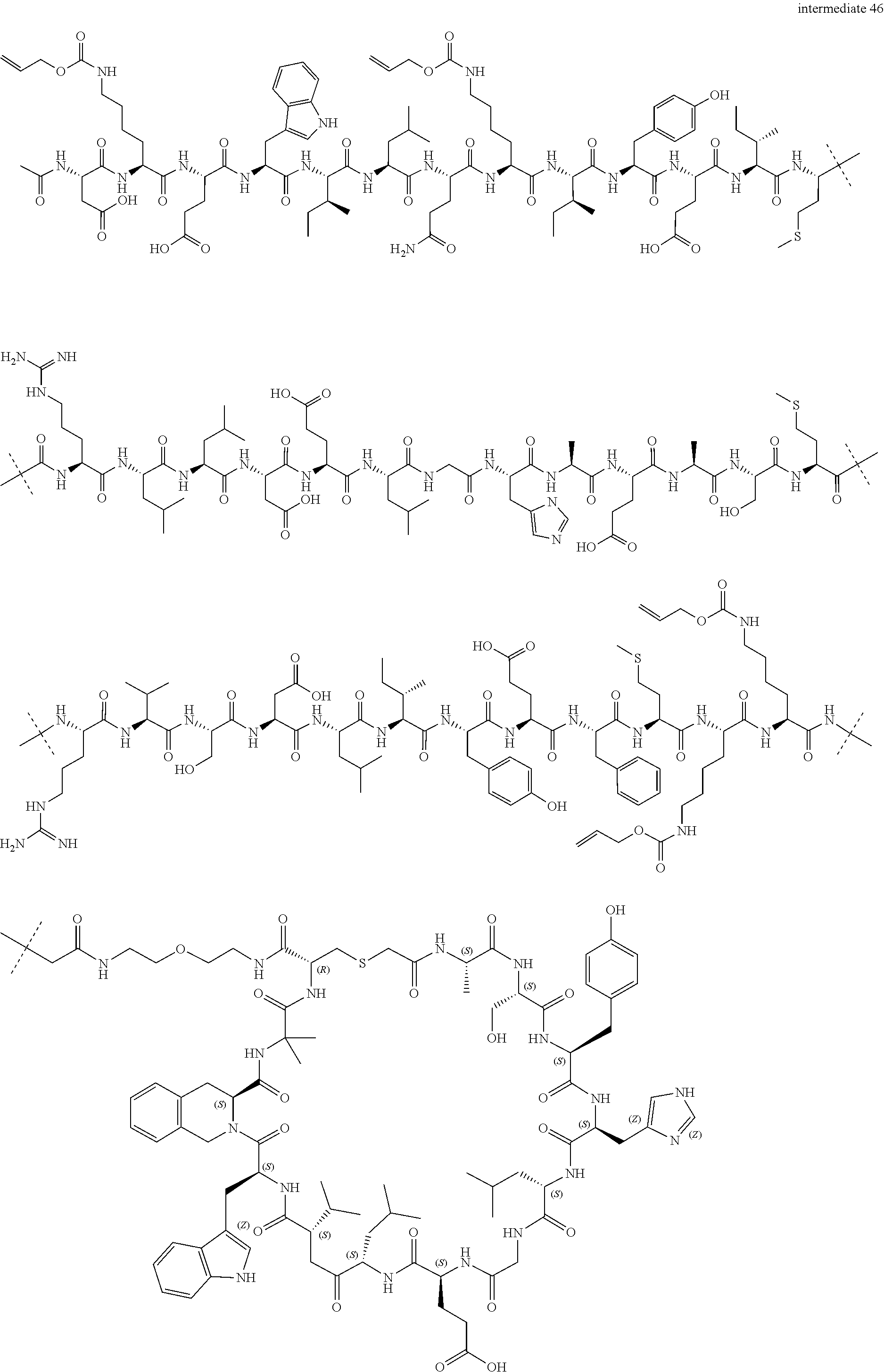

Preparation of Compound 159 (de-Alloc).

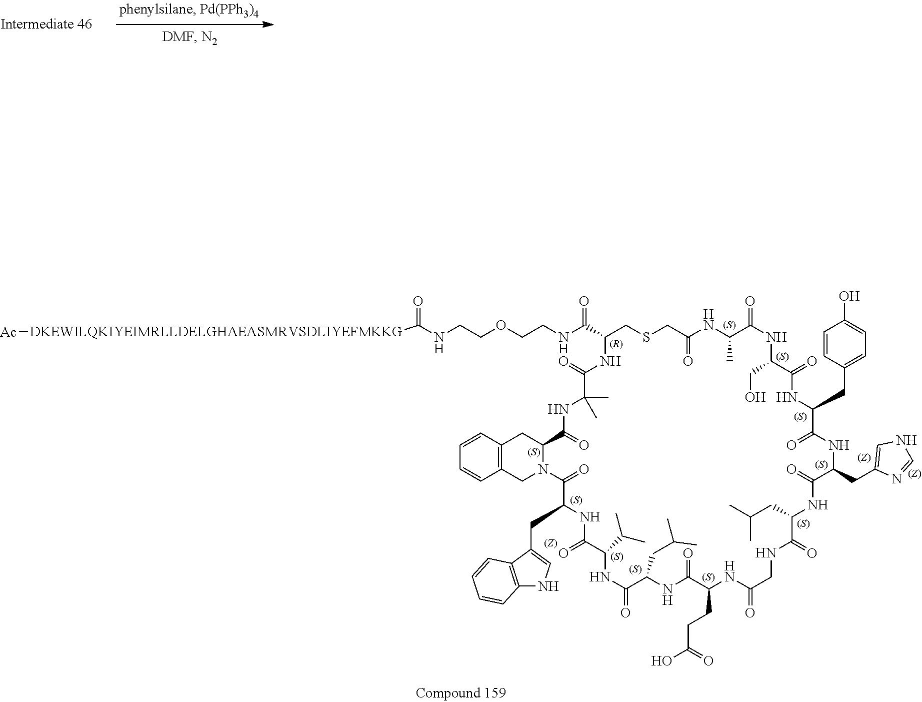

A mixture of intermediate 46 (97.0 mg, 14.46 mol, 1.00 equiv.) (SEQ ID NO:53), phenylsilane (31.29 mg, 289.18 mol, 35.6 uL, 20.00 equiv.), Pd(PPh₃)₄ (8.35 mg, 7.23 mol, 0.50 equiv.) in DMF (1 mL) was stirred under N₂ atmosphere at 20° C. for 0.5 h. The mixture was purified by prep-HPLC (A: 0.075% TFA/H₂O, B: MeCN) directly, then lyophilized to afford Compound 159 (28.7 mg, 91.2% purity, 28.4% yield) (SEQ ID NO:54) as a white solid. LCMS: RT=1.03 min, MS cal.: 6372.37, mass observed: [M+4H]⁴⁺+=1593.80, [M+5H]⁵⁺=1275.08, [M+6H]⁶⁺=1063.04.

Compound 160, Compound 161) were synthesized using the same method as Compound 159 (SEQ ID NO:54).

0.30 mmol resin afforded Compound 160 (Batch 1: 12.2 mg, 81.7% purity; Batch 2: 10.6 mg, 90.4% purity; 1.9% yield in total) as a white solid. LCMS: RT=1.02 min, MS cal.: 7104.24, [M+4H]⁴⁺+=1776.60, [M+5H]⁵⁺=1421.68, [M+6H]⁶⁺=1184.80.

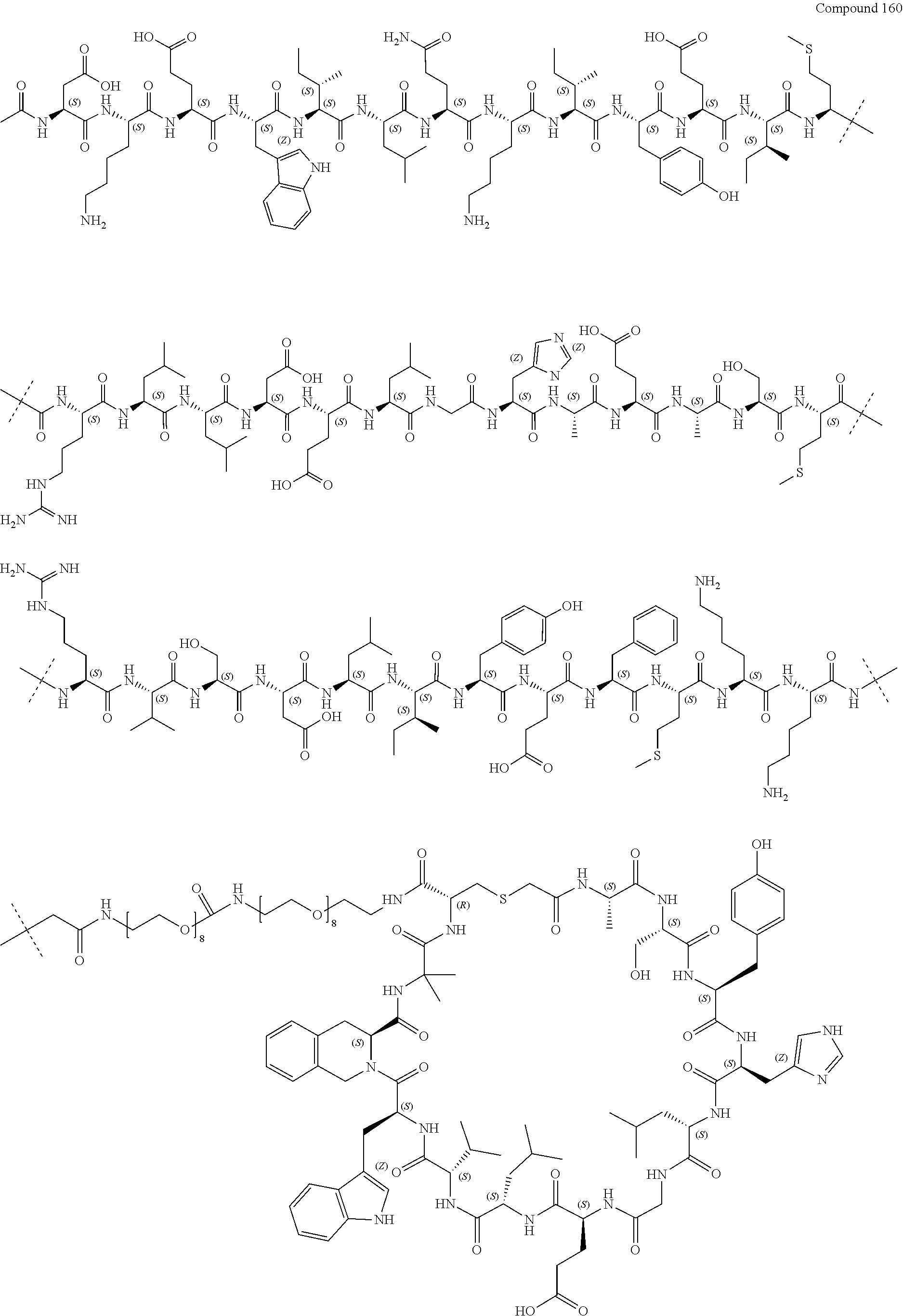

0.30 mmol resin afforded Compound 161 (Batch 1: 6.3 mg, 96.7% purity; Batch 2: 31.5 mg, 92.6% purity; 1.9% total yield) as a white solid. LCMS: RT=1.05 min, MS cal.: 6680.74, [M+4H]⁴⁺+=1670.90, [M+5H]⁵⁺=1137.05, [M+6H]⁶⁺=1114.49.

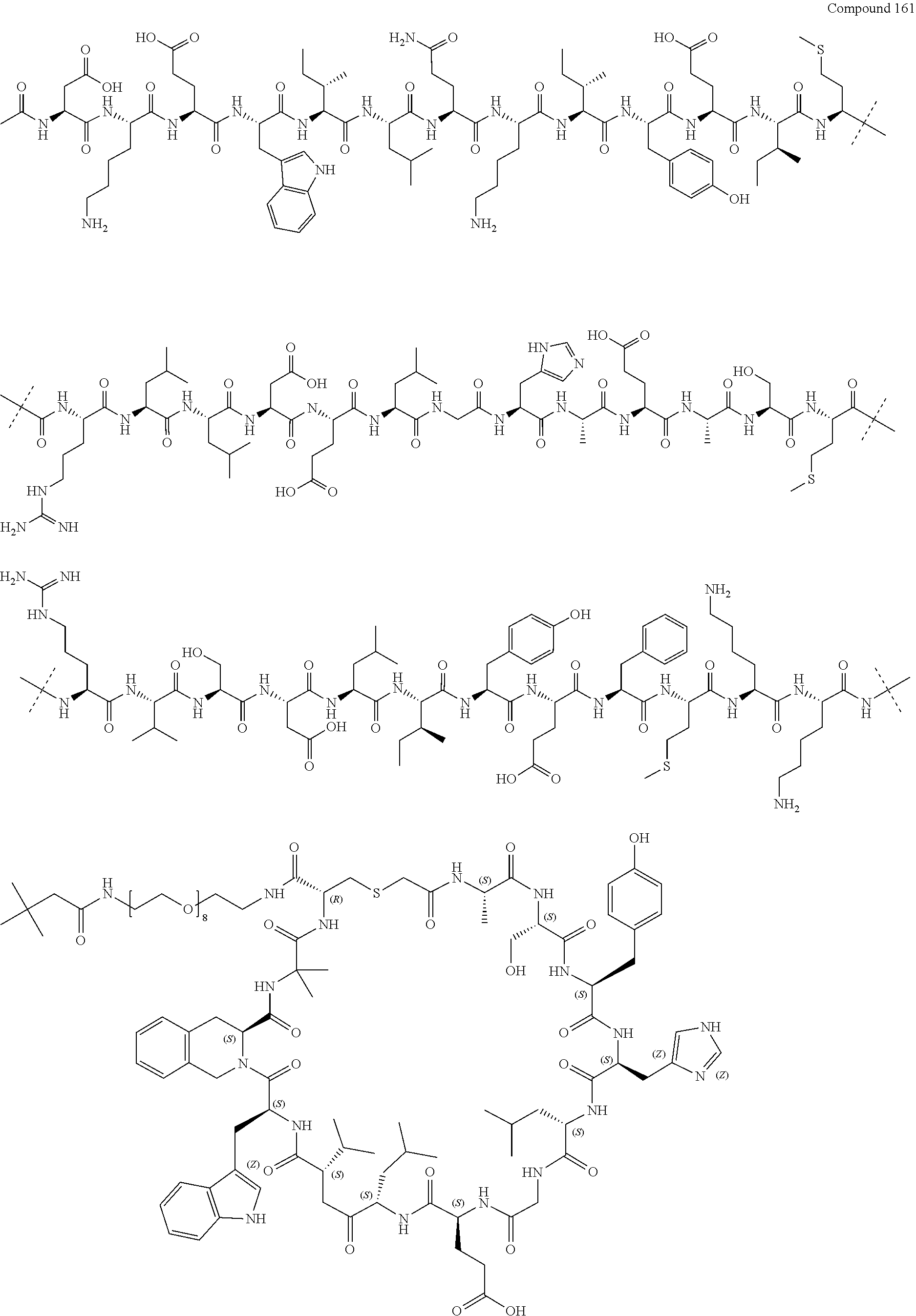

### Example 13. Procedure for Preparation of Compound 162 (Thioether Cyclic Peptide)

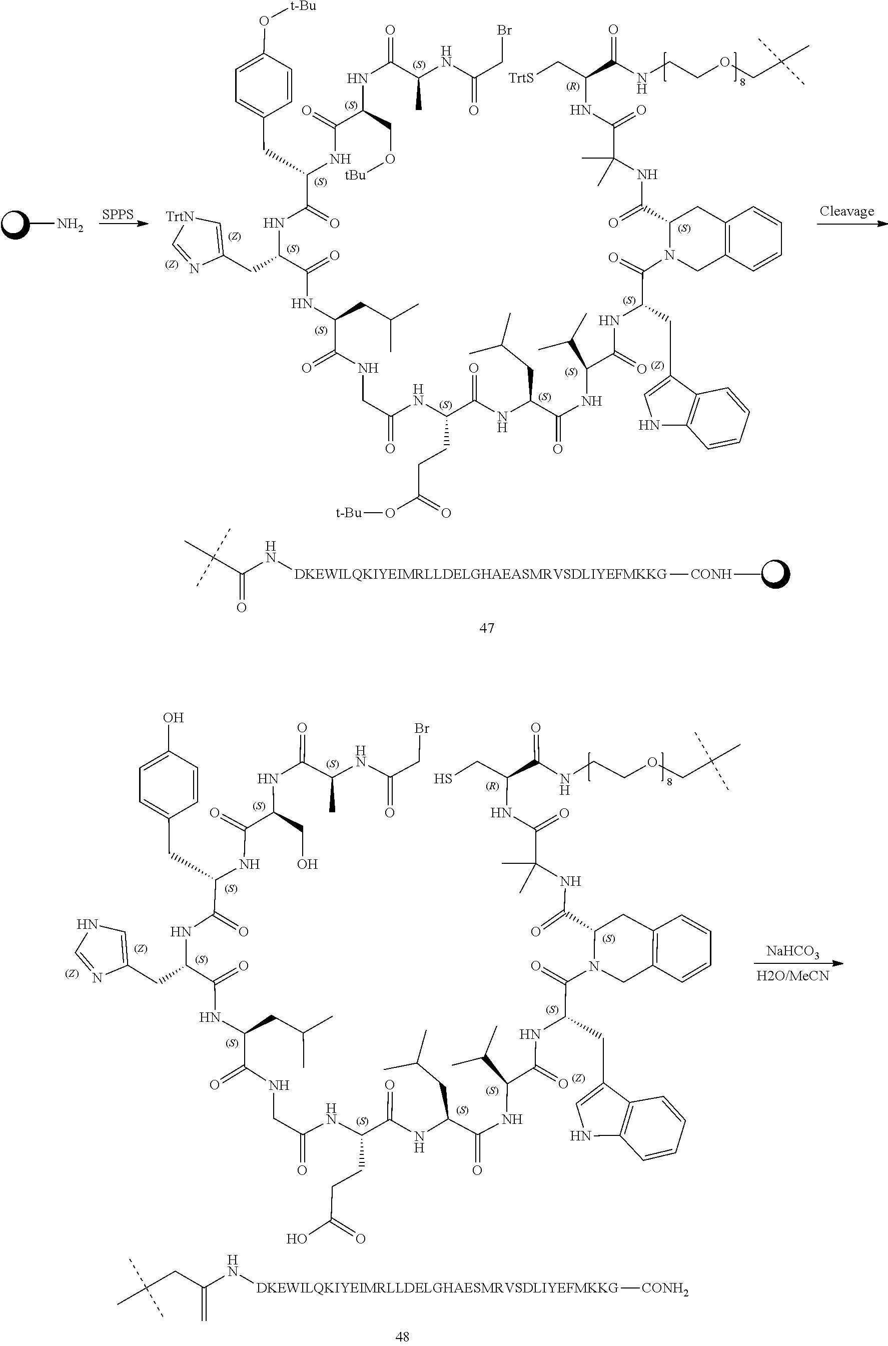

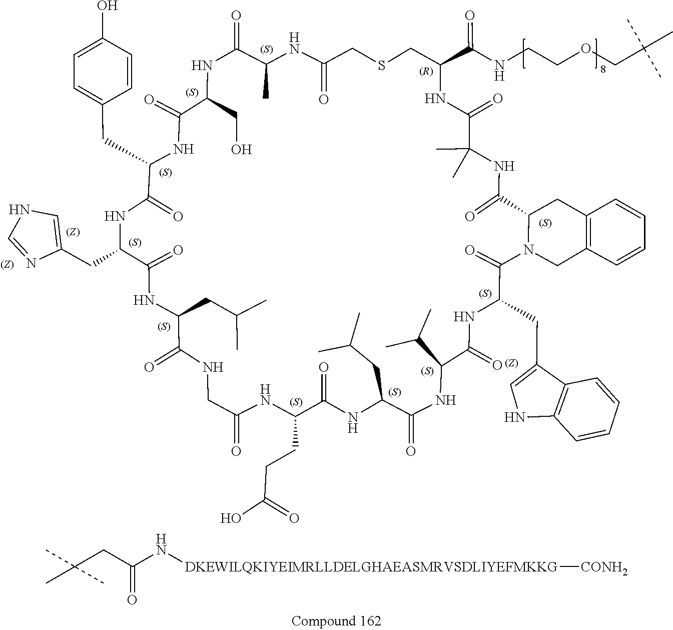

Peptide was synthesized using standard Fmoc chemistry (Rink AM resin).
1) Resin preparation: To the vessel containing Rink Amide AM resin (1.56 g, 0.50 mmol, 0.32 mmol/g) and DMF (20 mL) was bubbled with N₂ for 2 h at 20° C. Then 20% piperidine in DMF (40 mL) was added and the mixture was bubbled with N₂ for 30 min at 20° C. The mixture was filtered and washed with DMF (20 mL)*5 before proceeding to next step.
2) Coupling: A solution of Fmoc-Gly-OH (0.45 g, 3.00 equiv.), HBTU (0.54 g, 2.85 equiv.) in DMF (20 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (40 mL)*5.
3) Deprotection: 20% piperidine in DMF (40 mL) was added to the resin and the mixture was bubbled with N₂ for 30 min at 20° C. The deprotection reaction was monitored by ninhydrin test, if it showed blue or brownish red, the reaction was completed. The resin was then washed with DMF (40 mL)*5. 4) Steps 2 and 3 were repeated for the following amino acids elongation: Number #3□17, Table 11.
5) Coupling: A solution of Fmoc-Ala-OH (0.46 g, 3.00 equiv.), HATU (0.54 g, 2.85 equiv.) in DMF (20 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (60 mL)*5.
6) Step 3 was repeated for Fmoc deprotection. 7) Steps 5 and 6 were repeated for the following amino acids elongation: Number #19-40, Table 11. 8) The resin was then washed with DMF (40 mL)*5, MeOH (40 mL)*3, then dried under reduced pressure.

TABLE 11
The list of amino acids and the corresponding reagents used on SPPS

| # | Materials | Coupling reagents |
|---|---|---|
| 1 | Fmoc-Gly-OH (1.00 equiv.) | HBTU (2.85 equiv.) and DIEA (6.00 equiv.) |
| 2 | Fmoc-Lys(Boc)-OH (3.00 equiv.) | HBTU (2.85 equiv.) and DIEA (6.00 equiv.) |
| 3 | Fmoc-Lys(Boc)-OH (3.00 equiv.) | HBTU (2.85 equiv.) and DIEA (6.00 equiv.) |
| 4 | Fmoc-Met-OH (3.00 equiv.) | HBTU (2.85 equiv.) and DIEA (6.00 equiv.) |
| 5 | Fmoc-Phe-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Tyr(/Bu)-OH (3.00 equiv.) | HBTU (2.85 equiv.) and DIEA (6.00 equiv.) |
| 8 | Fmoc-Ile-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-Asp(OrBu)-OH (3.00 equiv.) | HBTU (2.85 equiv.) and DIEA (6.00 equiv.) |
| 11 | Fmoc-Ser(tBu)-OH (3.00 equiv.) | HBTU (2.85 equiv.) and DIEA (6.00 equiv.) |
| 12 | Fmoc-Val-OH (3.00 equiv.) | HBTU (2.85 equiv.) and DIEA (6.00 equiv.) |
| 13 | Fmoc-Arg(Pbf)-OH (3.00 equiv.) | HBTU (2.85 equiv.) and DIEA (6.00 equiv.) |
| 14 | Fmoc-Met-OH (3.00 equiv.) | HBTU (2.85 equiv.) and DIEA (6.00 equiv.) |
| 15 | Fmoc-Ser(?Bu)-OH (3.00 equiv.) | HBTU (2.85 equiv.) and DIEA (6.00 equiv.) |
| 16 | Fmoc-Ala-OH (3.00 equiv.) | HBTU (2.85 equiv.) and DIEA (6.00 equiv.) |
| 17 | Fmoc-Glu(OtBu)-OH (3.00 equiv.) | HBTU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-His(Trt)-OH (3.00 equiv.) | HATU (2.85 equiv.) and DIEA (6.00 equiv.) |
| 20 | Fmoc-Gly-OH (3.00 equiv.) | HATU (2.85 equiv.) and DIEA (6.00 equiv.) |
| 21 | Fmoc-Leu-OH (3.00 equiv.) | HATU (2.85 equiv.) and DIEA (6.00 equiv.) |
| 22 | Fmoc-Glu(OtBu)-OH (3.00 equiv.) | HATU (2.85 equiv.) and DIEA (6.00 equiv.) |
| 23 | Fmoc-Asp(OtBu)-OH (3.00 equiv.) | HATU (2.85 equiv.) and DIEA (6.00 equiv.) |
| 24 | Fmoc-Leu-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Arg(Pbf)-OH (3.00 equiv.) | HATU (2.85 equiv.) and DIEA (6.00 equiv.) |
| 27 | Fmoc-Met-OH (3.00 equiv.) | HATU (2.85 equiv.) and DIEA (6.00 equiv.) |
| 28 | Fmoc-Ile-OH (3.00 equiv.) | HATU (2.85 equiv.) and DIEA (6.00 equiv.) |
| 29 | Fmoc-Glu(OtBu)-OH (3.00 equiv.) | HATU (2.85 equiv.) and DIEA (6.00 equiv.) |
| 30 | Fmoc-Tyr((Bu)-OH (3.00 equiv.) | HATU (2.85 equiv.) and DIEA (6.00 equiv.) |
| 31 | Fmoc-Ile-OH (3.00 equiv.) | HATU (2.85 equiv.) and DIEA (6.00 equiv.) |
| 32 | Fmoc-Lys(Boc)-OH (3.00 equiv.) | HATU (2.85 equiv.) and DIEA (6.00 equiv.) |
| 33 | Fmoc-Gln(Trt)-OH (3.00 equiv.) | HATU (2.85 equiv.) and DIEA (6.00 equiv.) |
| 34 | Fmoc-Leu-OH (3.00 equiv.) | HATU (2.85 equiv.) and DIEA (6.00 equiv.) |
| 35 | Fmoc-Ile-OH (3.00 equiv.) | HATU (2.85 equiv.) and DIEA (6.00 equiv.) |
| 36 | Fmoc-Trp-OH (3.00 equiv.) | HATU (2.85 equiv.) and DIEA (6.00 equiv.) |
| 37 | Fmoc-Glu(OrBu)-OH (3.00 equiv.) | HATU (2.85 equiv.) and DIEA (6.00 equiv.) |
| 38 | Fmoc-Lys(Boc)-OH (3.00 equiv.) | HATU (2.85 equiv.) and DIEA (6.00 equiv.) |
| 39 | Fmoc-Asp(OtBu)-OH (3.00 equiv.) | HATU (2.85 equiv.) and DIEA (6.00 equiv.) |
| 40 | Fmoc-PEG8-CH2CH2COOH (3.00 | HATU (1.42 equiv.) and DIEA (3.00 equiv.) |
| | equiv.) | |
| 41 | Fmoc-Cys(Trt)-OH (3.00 equiv.) | HATU (2.85 equiv.) and DIEA (6.00 equiv.) |
| 42 | Fmoc-Aib-OH (3.00 equiv.) | HATU (2.85 equiv.) and DIEA (6.00 equiv.) |
| 43 | Fmoc-Tic-OH (3.00 equiv.) | HATU (2.85 equiv.) and DIEA (6.00 equiv.) |
| 44 | Fmoc-Trp-OH (3.00 equiv.) | HATU (2.85 equiv.) and DIEA (6.00 equiv.) |
| 45 | Fmoc-Val-OH (3.00 equiv.) | HATU (2.85 equiv.) and DIEA (6.00 equiv.) |
| 46 | Fmoc-Leu-OH (3.00 equiv.) | HATU (2.85 equiv.) and DIEA (6.00 equiv.) |
| 47 | Fmoc-Glu(OtBu)-OH (3.00 equiv.) | HATU (2.85 equiv.) and DIEA (6.00 equiv.) |
| 48 | Fmoc-Gly-OH (3.00 equiv.) | HATU (2.85 equiv.) and DIEA (6.00 equiv.) |
| 49 | Fmoc-Leu-OH (3.00 equiv.) | HATU (2.85 equiv.) and DIEA (6.00 equiv.) |
| 50 | Fmoc-His(Trt)-OH (3.00 equiv.) | HATU (2.85 equiv.) and DIEA (6.00 equiv.) |
| 51 | Fmoc-Tyr(tBu)-OH (3.00 equiv.) | HATU (2.85 equiv.) and DIEA (6.00 equiv.) |
| 52 | Fmoc-Ser(+Bu)-OH (3.00 equiv.) | HATU (2.85 equiv.) and DIEA (6.00 equiv.) |
| 53 | Fmoc-Ala-OH (3.00 equiv.) | HATU (2.85 equiv.) and DIEA (6.00 equiv.) |
| 54 | 2-bromoacetic acid (6.00 equiv.) | DIC (3.00 equiv.) |

Peptide cleavage, cyclization, and purification.

Cleavage solution (TFA/TIS/H₂O, 95/2.5/2.5, v/v/v, 20 mL) was added to the flask containing the side-chain protected resin-bound peptide (intermediate 47 (SEQ ID NO:55), CTC resin, 0.50 mmol) at room temperature and stirred for 2 h. The filtrate was collected. The peptide was precipitated with cold isopropyl ether (100 mL) and centrifuged (3 min at 3000 rpm).

The precipitate was washed with isopropyl ether (50 mL) twice, and the crude peptide was dried under reduced pressure for 2 h to afford intermediate 48 (SEQ ID NO:56) (2.5 g, crude) as a white solid.

To a mixture of intermediate 48 (2.5 g, crude) in MeCN/H₂O (5/5, v/v, 500 mL) was added NaHCO₃ to adjust pH=8, then the mixture was stirred at 20° C. for 1 h. The mixture was quenched by addition of 1 M HCl dropwise until the pH=6. After lyophilization, the crude was purified by prep-HPLC (0.075% TFA/H₂O, MeCN), followed by lyophilization to afford Compound 162 (SEQ ID NO:56) (batch 1: 17.1 mg, 95.7% purity; batch 2: 14.6 mg, 81.6% purity; 0.91% yield) as a white solid. LCMS: RT=1.06 min, MS cal.: 6666.71, mass observed: [M+4H]⁴⁺=1667.29, [M+5H]⁵⁺=1134.14, [M+6H]⁶⁺=1112.00, [M+7H]⁷⁺=952.90, [M+8H]⁸⁺=834.15.00, [M+9H]⁹⁺=741.47.

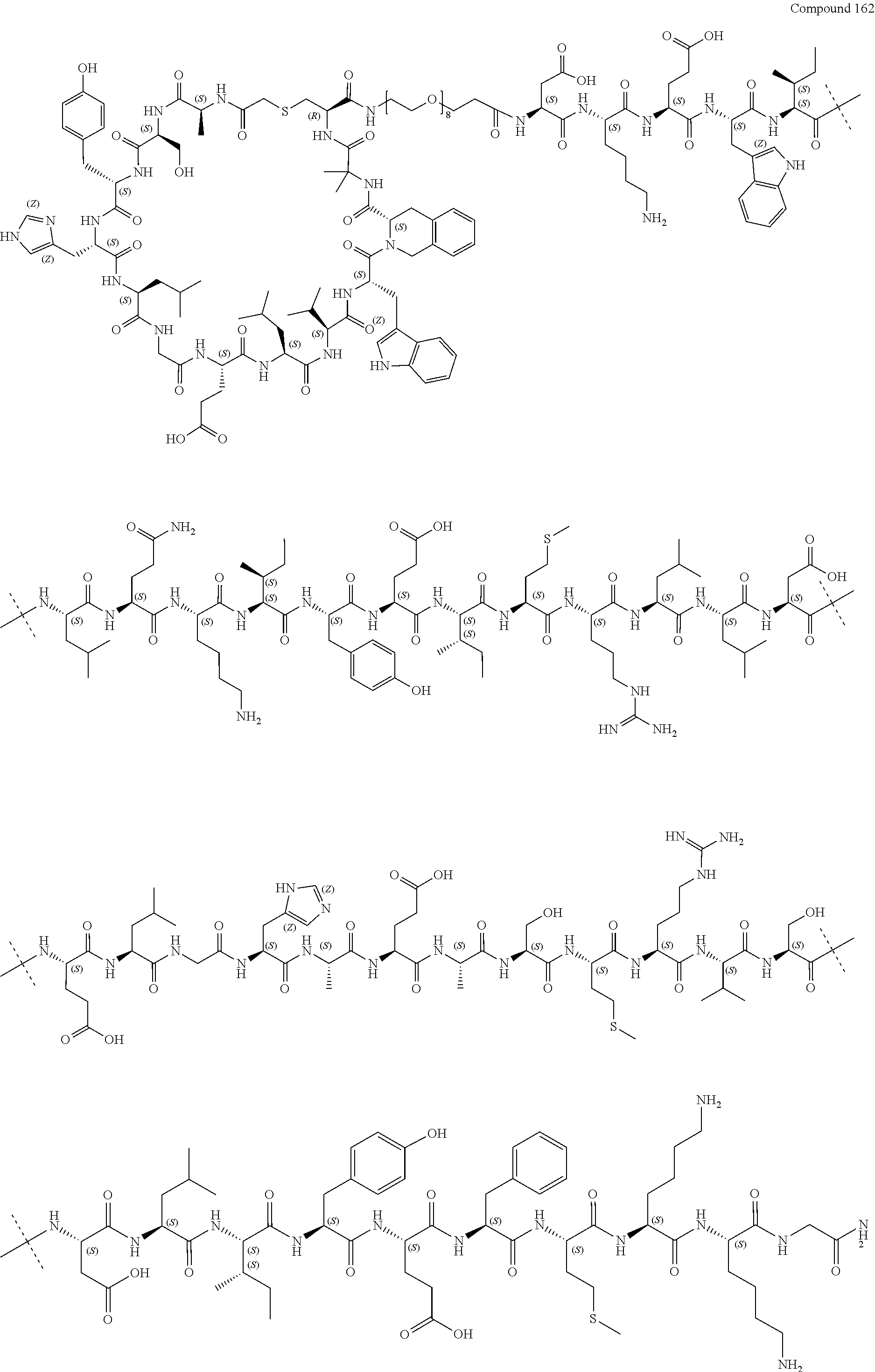

Thioether cyclic peptide, Compound 163 (compound 1174), was synthesized using the same method as Compound 162.

0.50 mmol resin afforded Compound 163 (12.4 mg, 82.8% purity, 0.32% yield) as a white solid. LCMS: RT=1.04 min, MS cal.: 6328.32, [M+4H]⁴⁺+=1582.93, [M+5H]⁵⁺=1266.66, [M+6H]⁶⁺=1055.30, [M+7H]⁷⁺=904.78, [M+8H]⁸⁺=791.70.

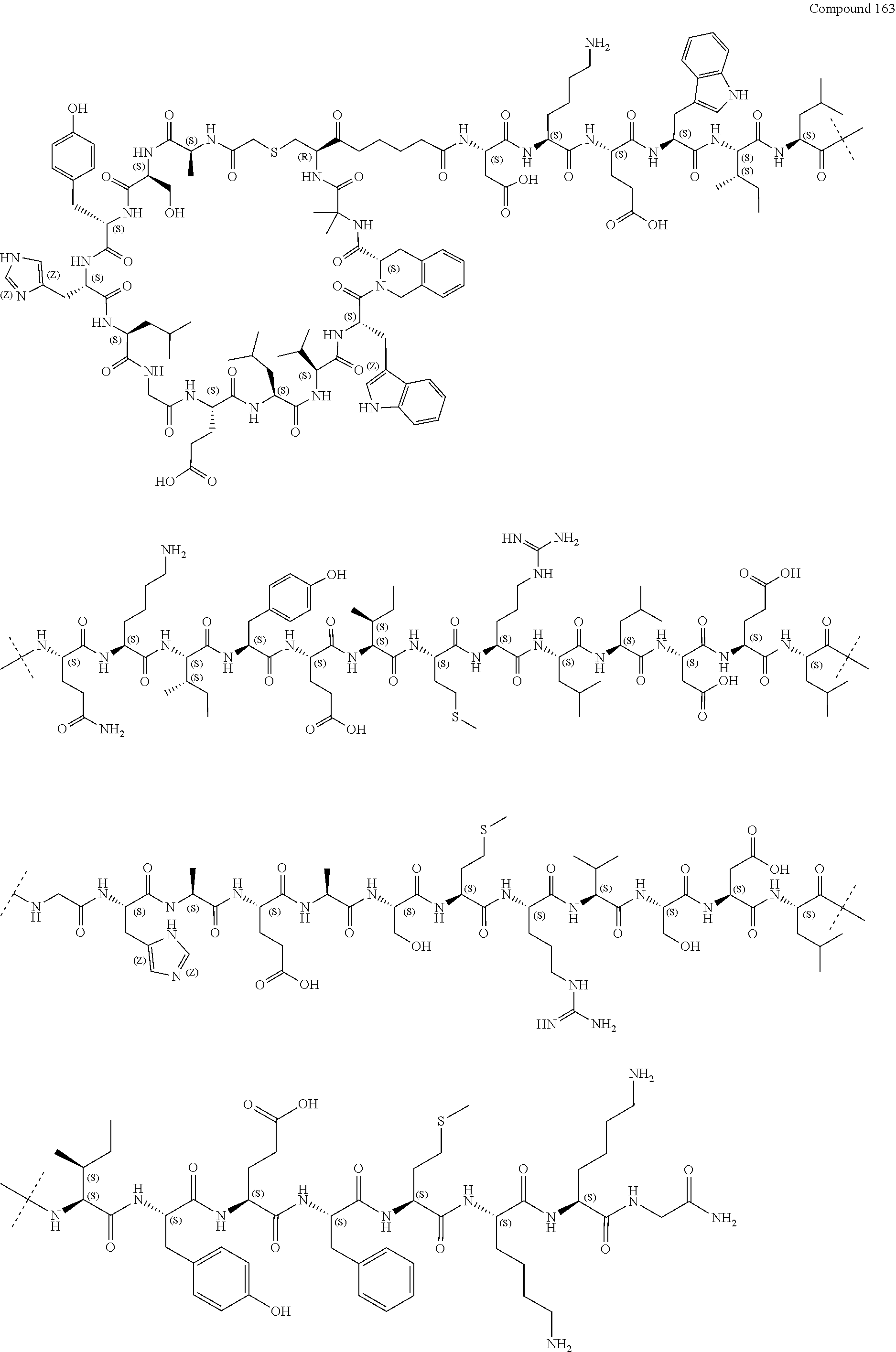

### Example 14. Preparation of Compound 164

Compound 164 was synthesized using the same method as Compound 139, which was performed by following the procedure mentioned in Example 8.

0.50 mmol rink amide AM resin afforded Compound 164 (Batch 1: 11.1 mg, 91.5% purity; Batch 2: 5.3 mg, 87.5% purity; 0.6% yield) as a white solid. LCMS: RT=2.03 min, MS cal.: 5473.36, observed mass: [M+3H]³⁺=1825.00, [M+4H]⁴⁺+=1369.10, [M+5H]⁵⁺=1095.6, [M+6H]⁶⁺=913.00.

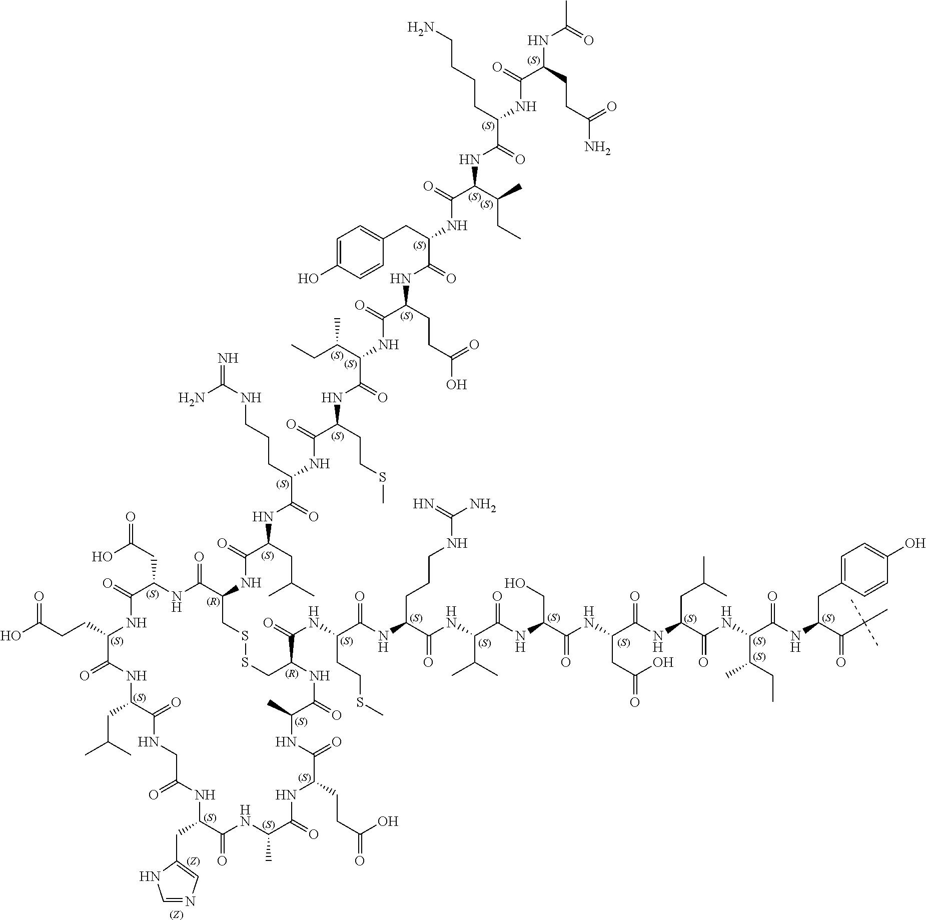

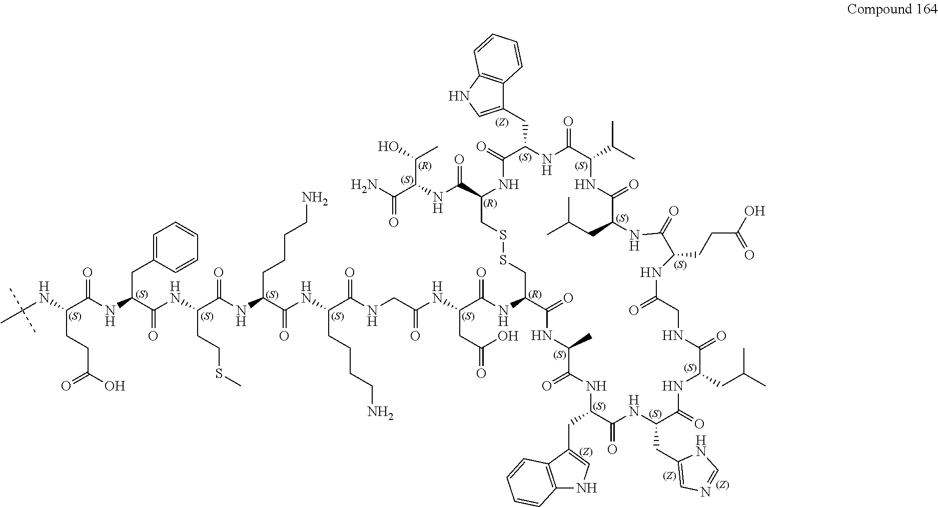

(SEQ ID NO: 57)
Ac-QKIYEIMRLCDELGHAEACMRVSDLIYEFMKKG-uABT-NH2

### Example 15. Procedure for Preparation of Compound 165

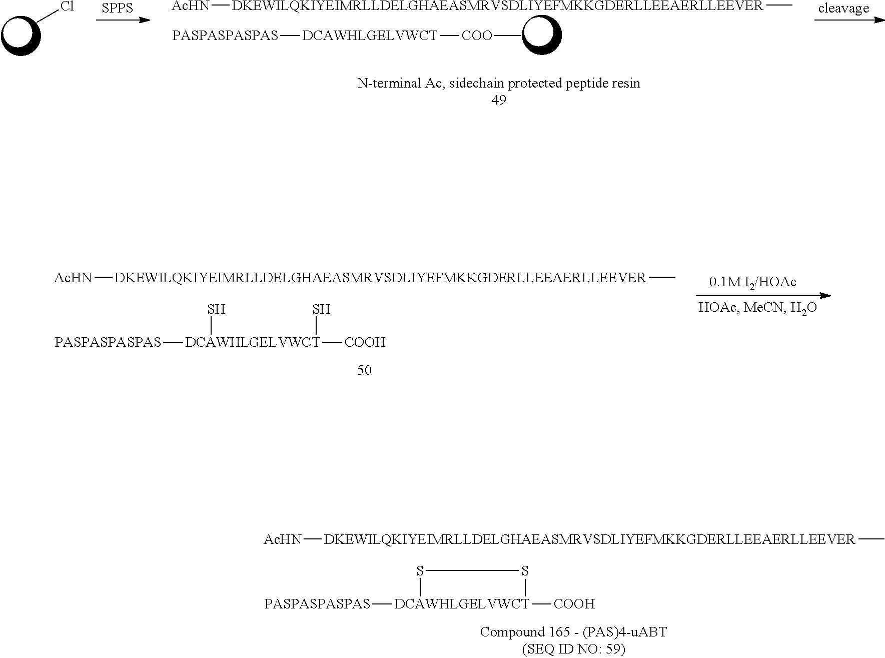

Peptide was synthesized using standard Fmoc chemistry (CTC resin).
1) Resin preparation: To the vessel containing CTC Resin (0.50 g, 0.50 mmol, 1.00 mmol/g) and Fmoc-Thr(tBu)-OH (198.0 mg, 0.50 mmol, 1.00 equiv.) in DCM (5 mL) was added DIEA (4.00 equiv.) dropwise and mixed for 2 h with N₂ bubbling at 20° C. Then MeOH (0.5 mL) was added and bubbled with N₂ for another 30 min. The resin was washed with DMF (10 mL)*5, followed by the addition of 20% piperidine in DMF (10 mL) and bubbled with N₂ for 30 min at 20° C. for Fmoc deprotection. The mixture was filtered and the resin was washed with DMF (10 mL)*5 before proceeding to next step.
2) Coupling: A solution of Fmoc-Cys(Trt)-OH (0.88 g, 3.00 equiv.), HBTU (0.54 g, 2.85 equiv.) in DMF (5 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (10 mL)*5.
3) Deprotection: 20% piperidine in DMF (10 mL) was added to the resin and the mixture was bubbled with N₂ for 30 min at 20° C. The deprotection reaction was monitored by ninhydrin test, if it showed blue or brownish red, the reaction was completed. The resin was then washed with DMF (10 mL)*5.
4) Steps 2 and 3 were repeated for the following amino acids elongation: Number #3-13, Table 12.
5) Coupling: A solution of Fmoc-Ser(tBu)-OH (0.58 g, 3.00 equiv.), HATU (0.54 g, 2.85 equiv.) in DMF (5 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (20 mL)*5.
6) Step 3 was repeated for Fmoc deprotection.
7) Steps 5 and 6 were repeated for the following amino acids elongation: Number #14-82, Table 12.
8) Acetylation: A solution of Ac₂O/NMM/DMF (2/1/17, v/v/v, 40 mL) was added to resin, the mixture was bubbled with N₂ for 20 min. The acetylation reaction was monitored by ninhydrin test. The resin was then washed with DMF (20 mL)*5, MeOH (20 mL)*3, then dried under reduced pressure.

TABLE 12
The list of amino acids and the corresponding reagents used on SPPS.

| # | Materials | Coupling reagents |
|---|---|---|
| 1 | Fmoc-Thr(+Bu)-OH (1.00 equiv.) | DIEA (4.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-His(Trt)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |
| 14 | Fmoc-Ser(/Bu)-OH (3.00 equiv.) | HATU (2.85 equiv.) and DIEA (6.00 equiv.) |
| 15 | Fmoc-Ala-OH (3.00 equiv.) | HATU (2.85 equiv.) and DIEA (6.00 equiv.) |
| 16 | Fmoc-Pro-OH (3.00 equiv.) | HATU (2.85 equiv.) and DIEA (6.00 equiv.) |
| 17 | Fmoc-Ser(tBu)-OH (3.00 equiv.) | HATU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-Pro-OH (3.00 equiv.) | HATU (2.85 equiv.) and DIEA (6.00 equiv.) |
| 20 | Fmoc-Ser(?Bu)-OH (3.00 equiv.) | HATU (2.85 equiv.) and DIEA (6.00 equiv.) |
| 21 | Fmoc-Ala-OH (3.00 equiv.) | HATU (2.85 equiv.) and DIEA (6.00 equiv.) |
| 22 | Fmoc-Pro-OH (3.00 equiv.) | HATU (2.85 equiv.) and DIEA (6.00 equiv.) |
| 23 | Fmoc-Ser(/Bu)-OH (3.00 equiv.) | HATU (2.85 equiv.) and DIEA (6.00 equiv.) |
| 24 | Fmoc-Ala-OH (3.00 equiv.) | HATU (2.85 equiv.) and DIEA (6.00 equiv.) |
| 25 | Fmoc-Pro-OH (3.00 equiv.) | HATU (2.85 equiv.) and DIEA (6.00 equiv.) |
| 26 | Fmoc-Arg(Pbf)-OH (1.00 equiv.) | HATU (2.85 equiv.) and DIEA (6.00 equiv.) |
| 27 | Fmoc-Glu(OtBu)-OH (3.00 equiv.) | HATU (2.85 equiv.) and DIEA (6.00 equiv.) |
| 28 | Fmoc-Val-OH (3.00 equiv.) | HATU (2.85 equiv.) and DIEA (6.00 equiv.) |
| 29 | Fmoc-Glu(OtBu)-OH (3.00 equiv.) | HATU (2.85 equiv.) and DIEA (6.00 equiv.) |
| 30 | Fmoc-Glu(OtBu)-OH (3.00 equiv.) | HATU (2.85 equiv.) and DIEA (6.00 equiv.) |
| 31 | Fmoc-Leu-OH (3.00 equiv.) | HATU (2.85 equiv.) and DIEA (6.00 equiv.) |
| 32 | Fmoc-Leu-OH (3.00 equiv.) | HATU (2.85 equiv.) and DIEA (6.00 equiv.) |
| 33 | Fmoc-Arg(Pbf)-OH (3.00 equiv.) | HATU (2.85 equiv.) and DIEA (6.00 equiv.) |
| 34 | Fmoc-Glu(OtBu)-OH (3.00 equiv.) | HATU (2.85 equiv.) and DIEA (6.00 equiv.) |
| 35 | Fmoc-Ala-OH (3.00 equiv.) | HATU (2.85 equiv.) and DIEA (6.00 equiv.) |
| 36 | Fmoc-Glu(OtBu)-OH (3.00 equiv.) | HATU (2.85 equiv.) and DIEA (6.00 equiv.) |
| 37 | Fmoc-Glu(OtBu)-OH (3.00 equiv.) | HATU (2.85 equiv.) and DIEA (6.00 equiv.) |
| 38 | Fmoc-Leu-OH (3.00 equiv.) | HATU (2.85 equiv.) and DIEA (6.00 equiv.) |
| 39 | Fmoc-Leu-OH (3.00 equiv.) | HATU (2.85 equiv.) and DIEA (6.00 equiv.) |
| 40 | Fmoc-Arg(Pbf)-OH (3.00 equiv.) | HATU (2.85 equiv.) and DIEA (6.00 equiv.) |
| 41 | Fmoc-Glu(OtBu)-OH (3.00 equiv.) | HATU (2.85 equiv.) and DIEA (6.00 equiv.) |
| 42 | Fmoc-Asp(OtBu)-OH (3.00 equiv.) | HATU (2.85 equiv.) and DIEA (6.00 equiv.) |
| 43 | Fmoc-Gly-OH (1.00 equiv.) | HATU (2.85 equiv.) and DIEA (6.00 equiv.) |
| 44 | Fmoc-Lys(Alloc)-OH (3.00 equiv.) | HATU (2.85 equiv.) and DIEA (6.00 equiv.) |
| 45 | Fmoc-Lys(Alloc)-OH (3.00 equiv.) | HATU (2.85 equiv.) and DIEA (6.00 equiv.) |
| 46 | Fmoc-Met-OH (3.00 equiv.) | HATU (2.85 equiv.) and DIEA (6.00 equiv.) |
| 47 | Fmoc-Phe-OH (3.00 equiv.) | HATU (2.85 equiv.) and DIEA (6.00 equiv.) |
| 48 | Fmoc-Glu(OtBu)-OH (3.00 equiv.) | HATU (2.85 equiv.) and DIEA (6.00 equiv.) |
| 49 | Fmoc-Tyr(/Bu)-OH (3.00 equiv.) | HATU (2.85 equiv.) and DIEA (6.00 equiv.) |
| 50 | Fmoc-Ile-OH (3.00 equiv.) | HATU (2.85 equiv.) and DIEA (6.00 equiv.) |
| 51 | Fmoc-Leu-OH (3.00 equiv.) | HATU (2.85 equiv.) and DIEA (6.00 equiv.) |
| 52 | Fmoc-Asp(OtBu)-OH (3.00 equiv.) | HATU (2.85 equiv.) and DIEA (6.00 equiv.) |
| 53 | Fmoc-Ser(tBu)-OH (3.00 equiv.) | HATU (2.85 equiv.) and DIEA (6.00 equiv.) |
| 54 | Fmoc-Val-OH (3.00 equiv.) | HATU (2.85 equiv.) and DIEA (6.00 equiv.) |
| 55 | Fmoc-Arg(Pbf)-OH (3.00 equiv.) | HATU (2.85 equiv.) and DIEA (6.00 equiv.) |
| 56 | Fmoc-Met-OH (3.00 equiv.) | HATU (2.85 equiv.) and DIEA (6.00 equiv.) |
| 57 | Fmoc-Ser(Bu)-OH (3.00 equiv.) | HATU (2.85 equiv.) and DIEA (6.00 equiv.) |
| 58 | Fmoc-Ala-OH (3.00 equiv.) | HATU (2.85 equiv.) and DIEA (6.00 equiv.) |
| 59 | Fmoc-Glu(OrBu)-OH (3.00 equiv.) | HATU (2.85 equiv.) and DIEA (6.00 equiv.) |
| 60 | Fmoc-Ala-OH (3.00 equiv.) | HATU (2.85 equiv.) and DIEA (6.00 equiv.) |
| 61 | Fmoc-His(Trt)-OH (3.00 equiv.) | HATU (2.85 equiv.) and DIEA (6.00 equiv.) |
| 62 | Fmoc-Gly-OH (3.00 equiv.) | HATU (2.85 equiv.) and DIEA (6.00 equiv.) |
| 63 | Fmoc-Leu-OH (3.00 equiv.) | HATU (2.85 equiv.) and DIEA (6.00 equiv.) |
| 64 | Fmoc-Glu(OtBu)-OH (3.00 equiv.) | HATU (2.85 equiv.) and DIEA (6.00 equiv.) |
| 65 | Fmoc-Asp(OtBu)-OH (3.00 equiv.) | HATU (2.85 equiv.) and DIEA (6.00 equiv.) |
| 66 | Fmoc-Leu-OH (3.00 equiv.) | HATU (2.85 equiv.) and DIEA (6.00 equiv.) |
| 67 | Fmoc-Leu-OH (3.00 equiv.) | HATU (2.85 equiv.) and DIEA (6.00 equiv.) |
| 68 | Fmoc-Arg(Pbf)-OH (3.00 equiv.) | HATU (2.85 equiv.) and DIEA (6.00 equiv.) |
| 69 | Fmoc-Met-OH (3.00 equiv.) | HATU (2.85 equiv.) and DIEA (6.00 equiv.) |
| 70 | Fmoc-Ile-OH (3.00 equiv.) | HATU (2.85 equiv.) and DIEA (6.00 equiv.) |
| 71 | Fmoc-Glu(OrBu)-OH (3.00 equiv.) | HATU (2.85 equiv.) and DIEA (6.00 equiv.) |
| 72 | Fmoc-Tyr((Bu)-OH (3.00 equiv.) | HATU (2.85 equiv.) and DIEA (6.00 equiv.) |
| 73 | Fmoc-Ile-OH (3.00 equiv.) | HATU (2.85 equiv.) and DIEA (6.00 equiv.) |
| 74 | Fmoc-Lys(Alloc)-OH (3.00 equiv.) | HATU (2.85 equiv.) and DIEA (6.00 equiv.) |
| 75 | Fmoc-Gln(Trt)-OH (3.00 equiv.) | HATU (2.85 equiv.) and DIEA (6.00 equiv.) |
| 76 | Fmoc-Leu-OH (3.00 equiv.) | HATU (2.85 equiv.) and DIEA (6.00 equiv.) |
| 77 | Fmoc-Ile-OH (3.00 equiv.) | HATU (2.85 equiv.) and DIEA (6.00 equiv.) |
| 78 | Fmoc-Trp-OH (3.00 equiv.) | HATU (2.85 equiv.) and DIEA (6.00 equiv.) |
| 79 | Fmoc-Glu(OtBu)-OH (3.00 equiv.) | HATU (2.85 equiv.) and DIEA (6.00 equiv.) |
| 80 | Fmoc-Lys(Alloc)-OH (3.00 equiv.) | HATU (2.85 equiv.) and DIEA (6.00 equiv.) |
| 81 | Fmoc-Asp(OtBu)-OH (3.00 equiv.) | HATU (2.85 equiv.) and DIEA (6.00 equiv.) |
| 82 | Acetylation | Ac2O/NMM/DMF (2/1/17, v/v/v) |

Peptide cleavage, cyclization and purification.
1) Cleavage solution (TFA/TIS/H₂O, 95/2.5/2.5, v/v/v, 170 mL) was added to the flask containing the side-chain protected resin-bound peptide intermediate 49 (SEQ ID NO: 58) (CTC resin, 8.72 g, 0.50 mmol) at room temperature and stirred for 2 h.
2) After filtration, the filtrate was collected. The filtrate was precipitated with cold isopropyl ether (800 mL). After filtration, the solid was washed with isopropyl ether (800 mL) twice, and the crude peptide was dried under reduced pressure for 2 h to afford intermediate 50 (SEQ ID NO:58) (4.52 g, crude) as a white solid.
3) To a mixture of intermediate 50 (4.52 g, crude) in MeCN/H₂O (1/1, v/v, 0.5 L) was added 0.1 M I₂/AcOH dropwise until a yellow color persisted, then the mixture was stirred at 20° C. for 5 min. The mixture was quenched by addition of 0.1 M aq. Na₂S₂O₃ dropwise until the yellow color disappeared. After filtration, the filtrate was purified by prep-HPLC (0.075% TFA/H₂O, MeCN), followed by lyophilization to afford Compound 165 (batch 1: 11.0 mg, 71.0% purity; batch 2: 31.5 mg, 59.5% purity; 0.91% yield) as a white solid. LCMS: RT=2.29 min, MS cal.: 9386.56, mass observed: [M+5H]⁵⁺=1878.6, [M+6H]⁶⁺=1565.3, [M+7H]⁷⁺=1342.2, [M+8H]⁸⁺=1174.6, [M+9H]⁹⁺=1044.0, [M+10H]¹⁰⁺=939.8, [M+11H]¹¹⁺=854.3, [M+12H]¹²⁺=783.7.

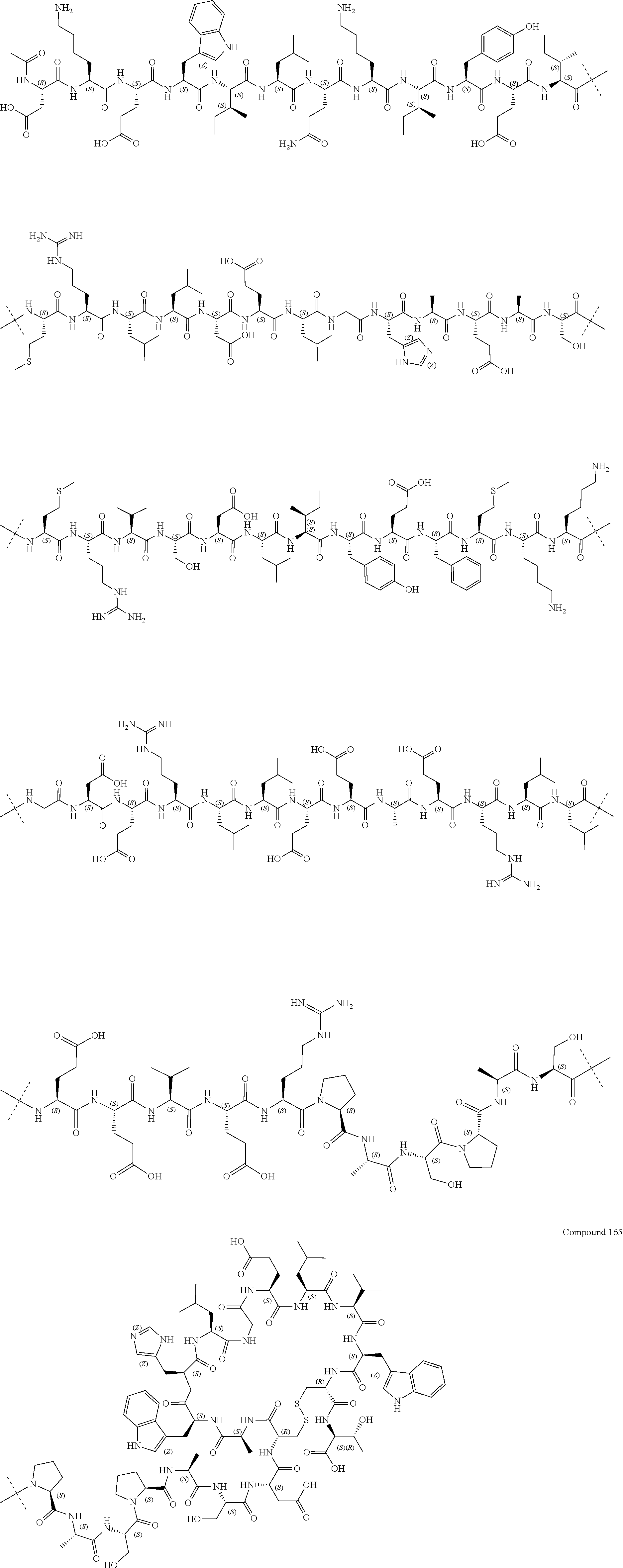

Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Pro-Ala-Ser-
Pro-Ala-Ser-Pro-Ala-Ser-Pro-Ala-Ser-Asp-Cyclo[Cys-
Ala-Trp-His-Leu-Gly-Glu-Leu-Val-Trp-Cys]-Thr-COOH

uABT disulfide cyclic peptides, Compounds 166, 167, and 168, were synthesized using the same method with Compound 165.

0.50 mmol resin afforded Compound 166 (batch 1: 12.0 mg, 85.2% purity; batch 2: 24.5 mg, 81.7% purity; 1.08% yield) as a white solid. LCMS: RT=2.16 min, MS cal.: 6711.62, mass observed: [M+4H]⁴⁺+=1678.7, [M+5H]⁵⁺=1343.3, [M+6H]⁶⁺=1119.8, [M+7H]⁷⁺=959.7, [M+8H]⁸⁺=840.4.

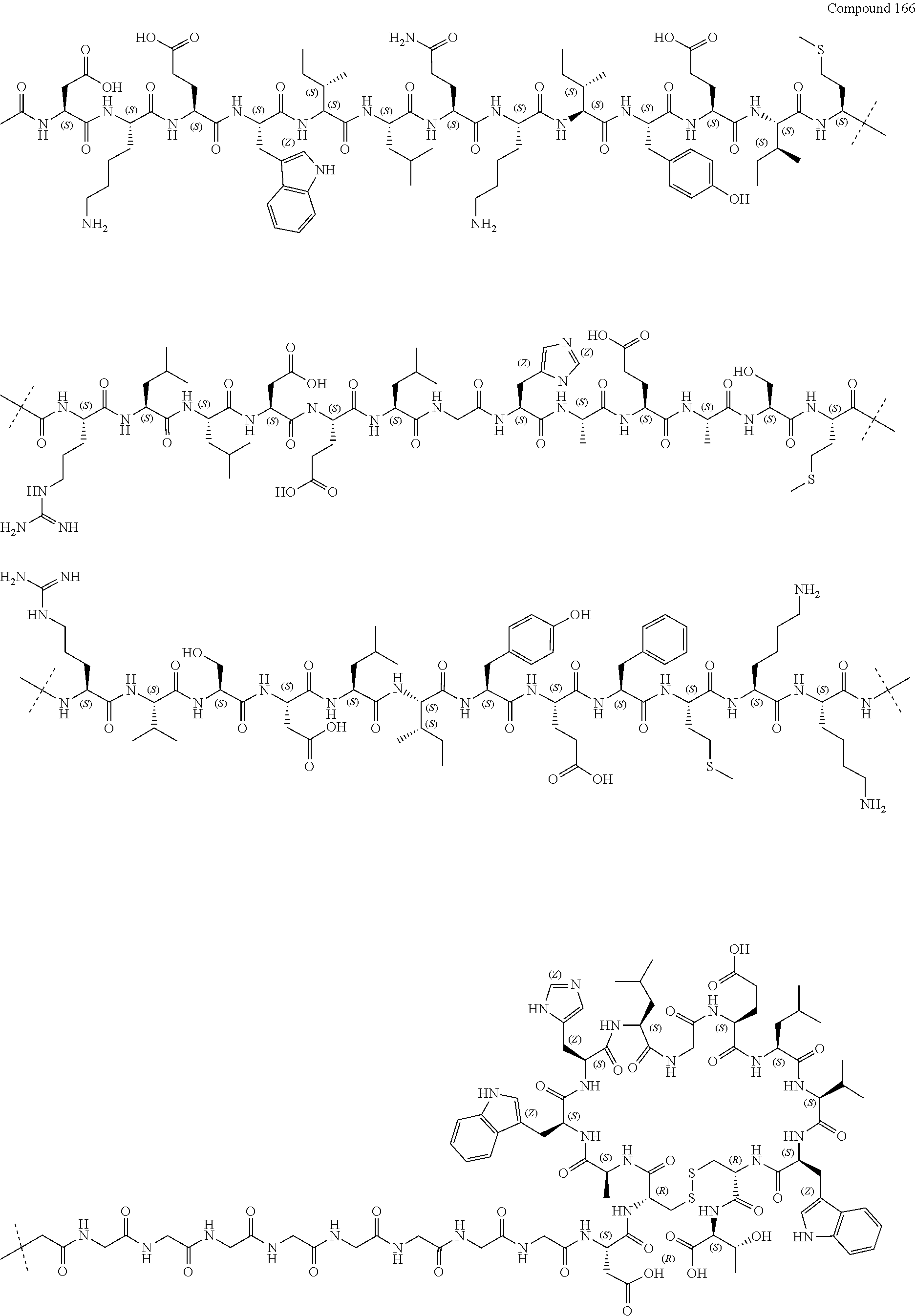

(SEQ ID NO: 60)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Gly-Gly-Gly-Gly-Gly-Gly-Gly-Gly-
Asp-Cyclo[Cys-Ala-Trp-His-Leu-Gly-Glu-Leu-Val-Trp-
Cys]-Thr-COOH

0.50 mmol resin afforded Compound 167 (44 mg, 95.4% purity, 1.20% yield) as a white solid. LCMS: RT=2.15 min, MS cal.: 7276.28, [M+4H]⁴⁺+=1820.0, [M+5H]⁵⁺=1456.2, [M+6H]⁶⁺=1213.6, [M+7H]⁷⁺=1040.4, [M+8H]⁸⁺=910.7, [M+9H]⁹⁺=809.2.

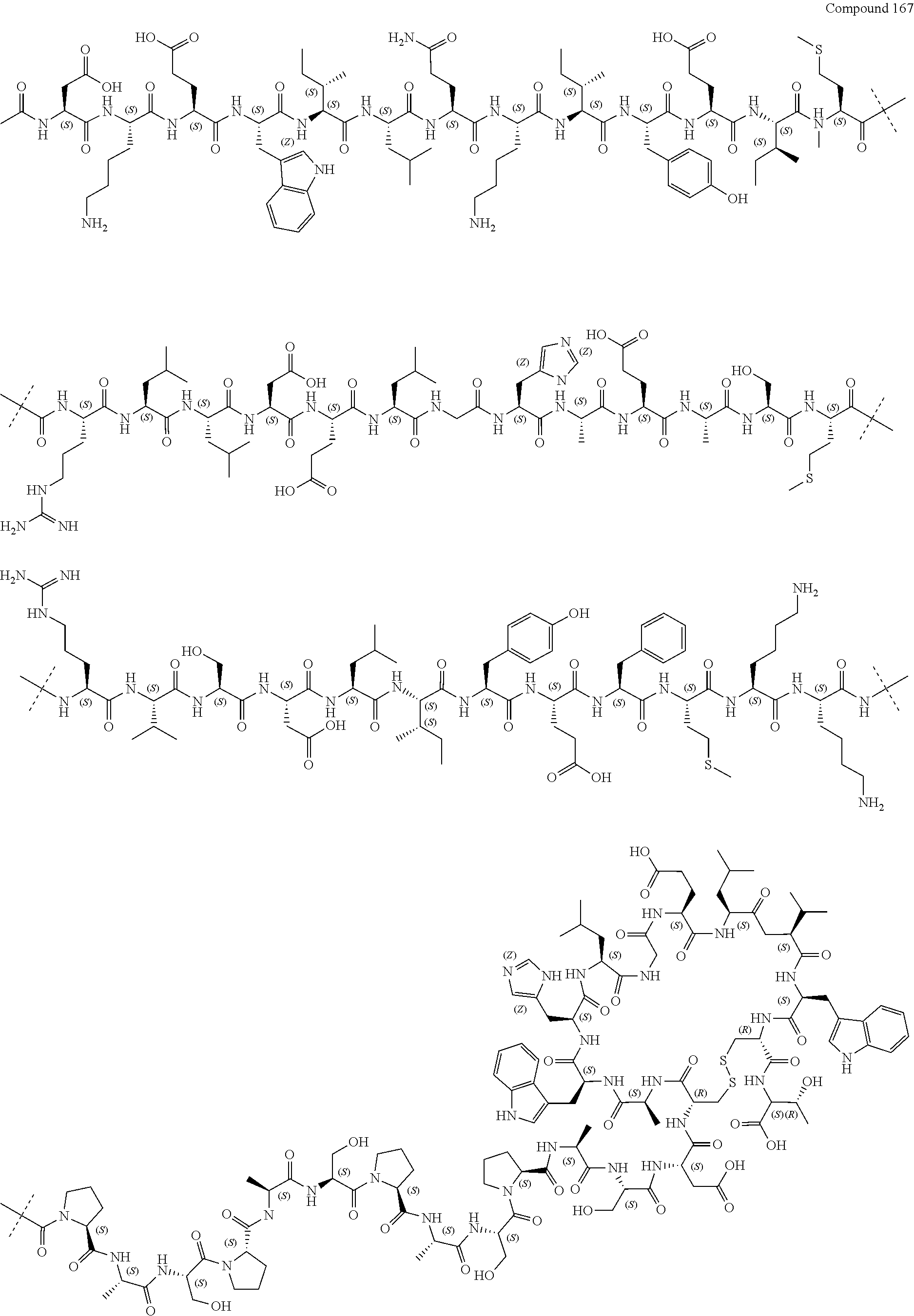

(SEQ ID NO: 61)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Pro-Ala-Ser-Pro-Ala-Ser-Pro-Ala-
Ser-Pro-Ala-Ser-Asp-Cyclo[Cys-Ala-Trp-His-Leu-Gly-
Glu-Leu-Val-Trp-Cys]-Thr-COOH

0.50 mmol resin afforded Compound 168 (126 mg, 90.9% purity, 3.34% yield) as a white solid. LCMS: RT=2.30 min, MS cal.: 7525.69, [M+5H]⁵⁺=1696.90, [M+6H]⁶⁺=1414.30, [M+7H]7=1212.30, [M+8H]⁸⁺=1060.90, [M+9H]⁹⁺=943.00, [M+10H]¹⁰⁺=848.80.

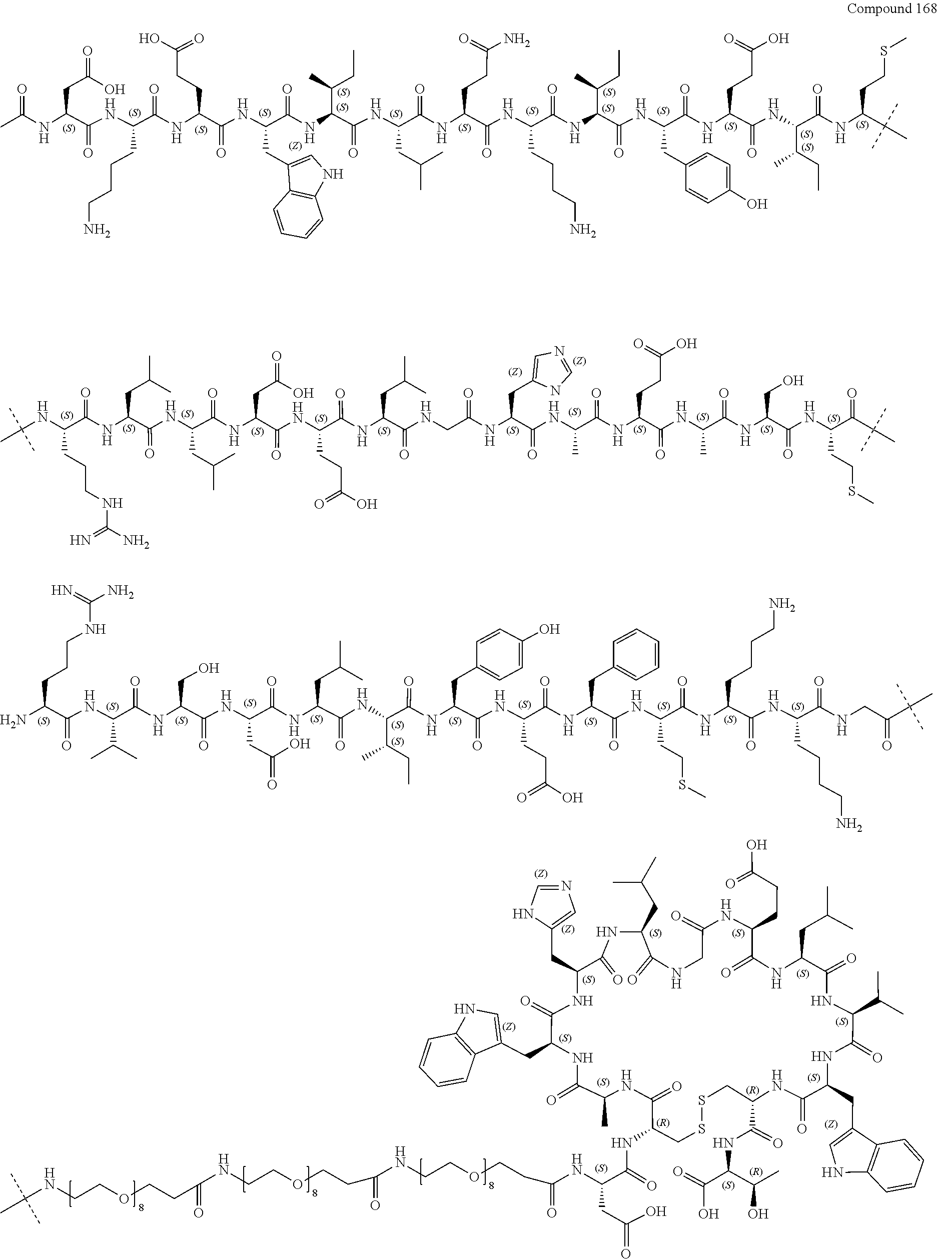

(SEQ ID NO: 62)
Ac-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-PEG8-PEG8-PEG8-Asp-Cyclo[Cys-Ala-
Trp-His-Leu-Gly-Glu-Leu-Val-Trp-Cys]-Thr-COOH

uABT disulfide cyclic peptides, Compounds 169, 170, 171, 172, 173, and 174 were synthesized using the same procedure as Compound 142. which was performed by following the procedure mentioned in Example 9.

0.5 mmol resin afforded Compound 169 (batch 1: 3.7 mg, 76.1% purity; batch 2: 8.0 mg, 72.0% purity; 0.27% yield) as a white solid. LCMS: RT=2.26 min, MS cal.: 8778.88, [M+5H]⁵⁺=1757.10, [M+6H]⁶⁺=1464.20, [M+7H]⁷⁺=1254.90, [M+8H]⁸⁺=1098.40, [M+9H]⁹⁺=976.50, [M+10H]¹⁰⁺=879.00.

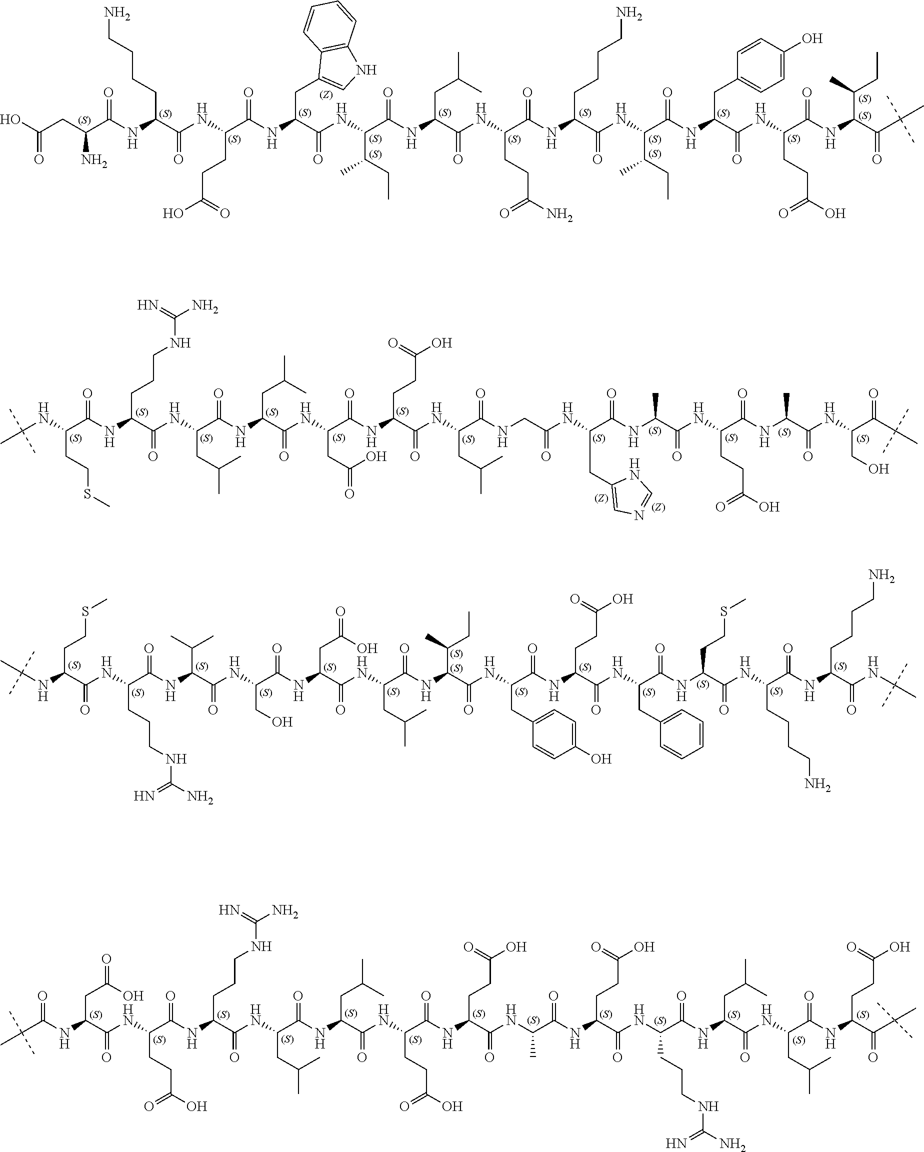

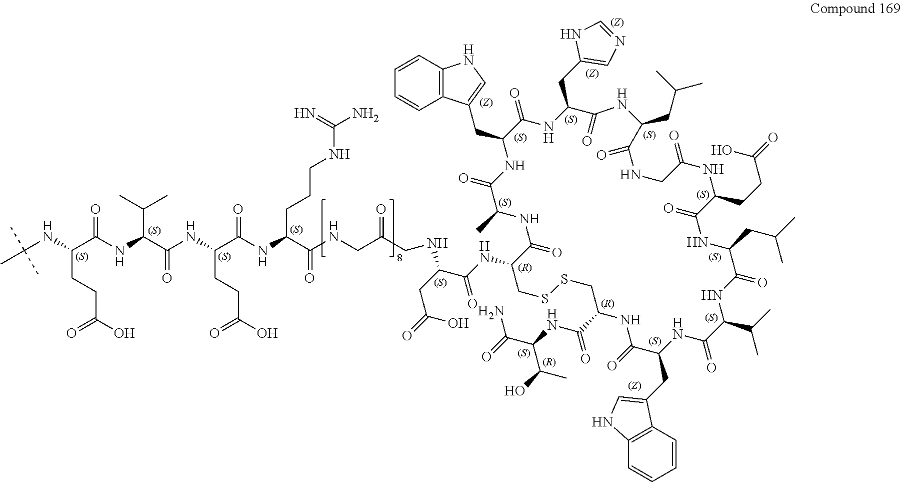

(SEQ ID NO: 63)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gly-Gly-
Gly-Gly-Gly-Gly-Gly-Asp-Cyclo[Cys-Ala-Trp-His-
Leu-Gly-Glu-Leu-Val-Trp-Cys]-Thr-NH2

0.5 mmol resin afforded Compound 170 (16.7 mg, 88.3% purity; 0.39% yield) as a white solid. LCMS: RT=2.28 min, MS cal.: 8664.77, [M+5H]⁵⁺=1734.10, [M+6H]⁶⁺=1445.20, [M+7H]⁷⁺=1238.90, [M+8H]⁸⁺=1083.90, [M+9H]⁹⁺=963.80, [M+10H]¹⁰⁺=867.70, [M+11H]¹¹⁺+=788.90.

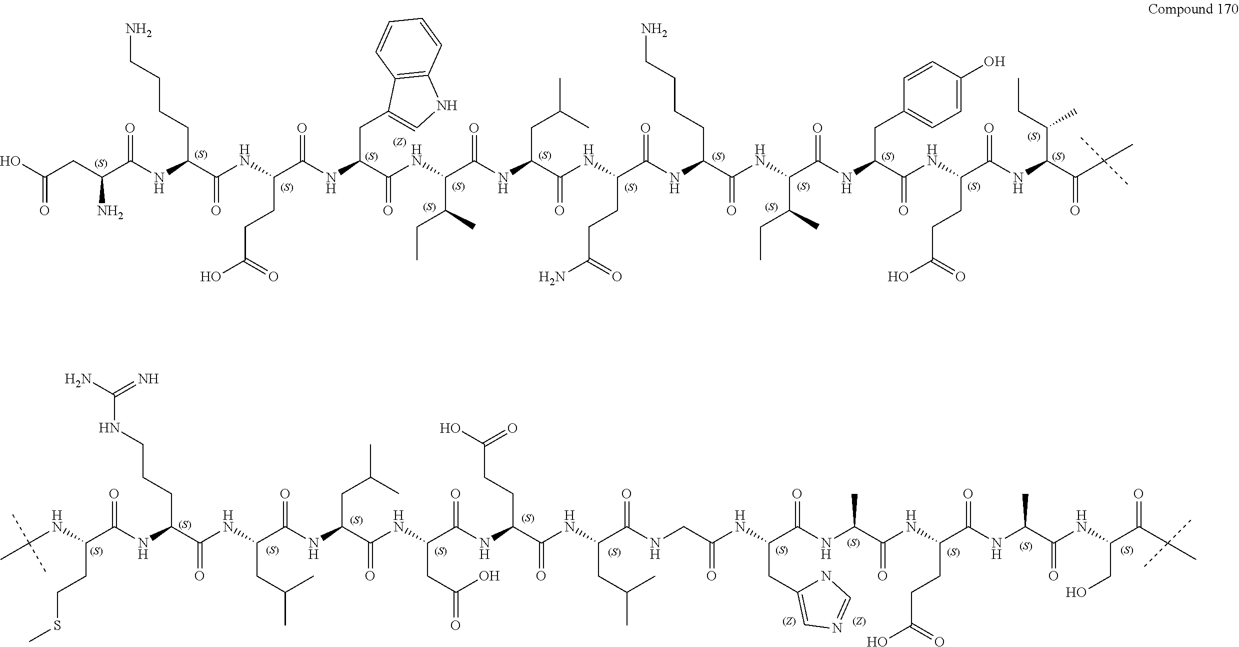

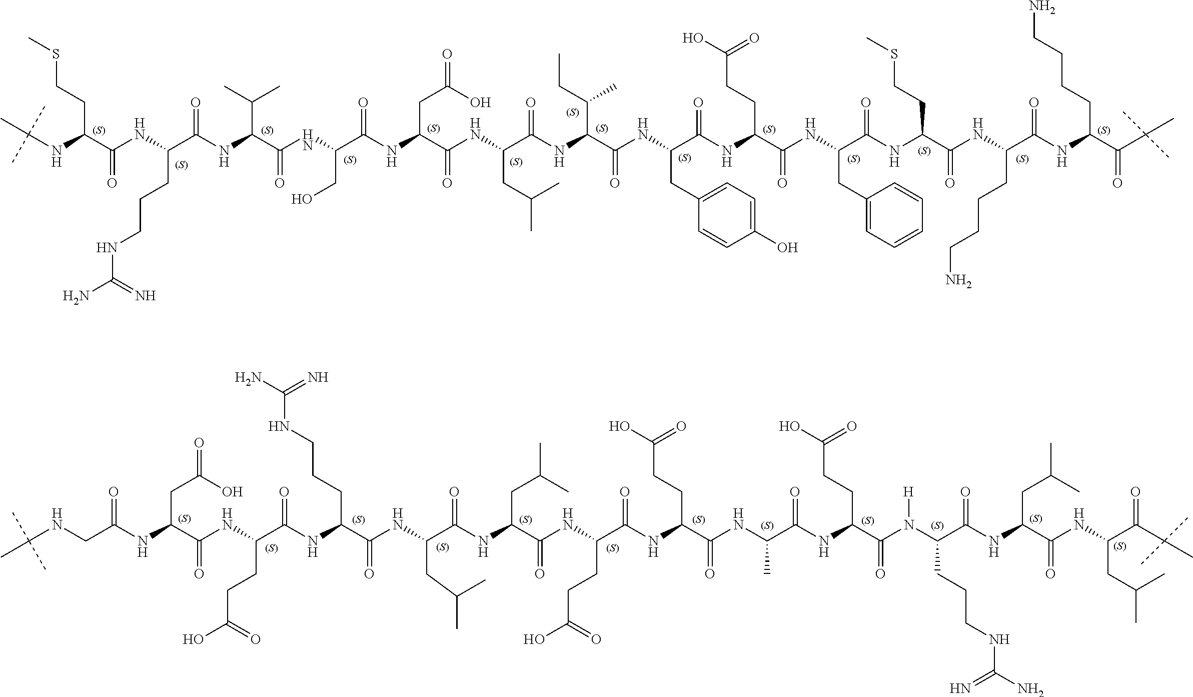

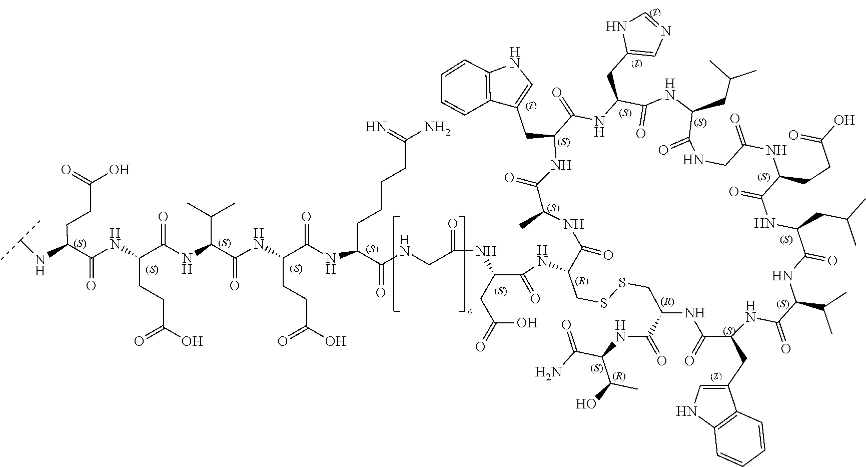

(SEQ ID NO: 64)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gly-Gly-
Gly-Gly-Gly-Asp-Cyclo[Cys-Ala-Trp-His-Leu-Gly-Glu-
Leu-Val-Trp-Cys]-Thr-NH2

0.5 mmol resin afforded Compound 171 (batch 1: 14.0 mg, 87.1% purity; batch 2: 16.2 mg, 78.4% purity; 0.35% yield) as a white solid. LCMS: RT=2.28 min, MS cal.: 8550.67, [M+5H]⁵⁺=1711.10, [M+6H]⁶⁺=1426.20, [M+7H]⁷⁺=1222.60, [M+8H]⁸⁺=1069.80, [M+9H]⁹⁺=951.20, [M+10H]¹⁰⁺=856.00, [M+11H]¹¹⁺+=778.80.

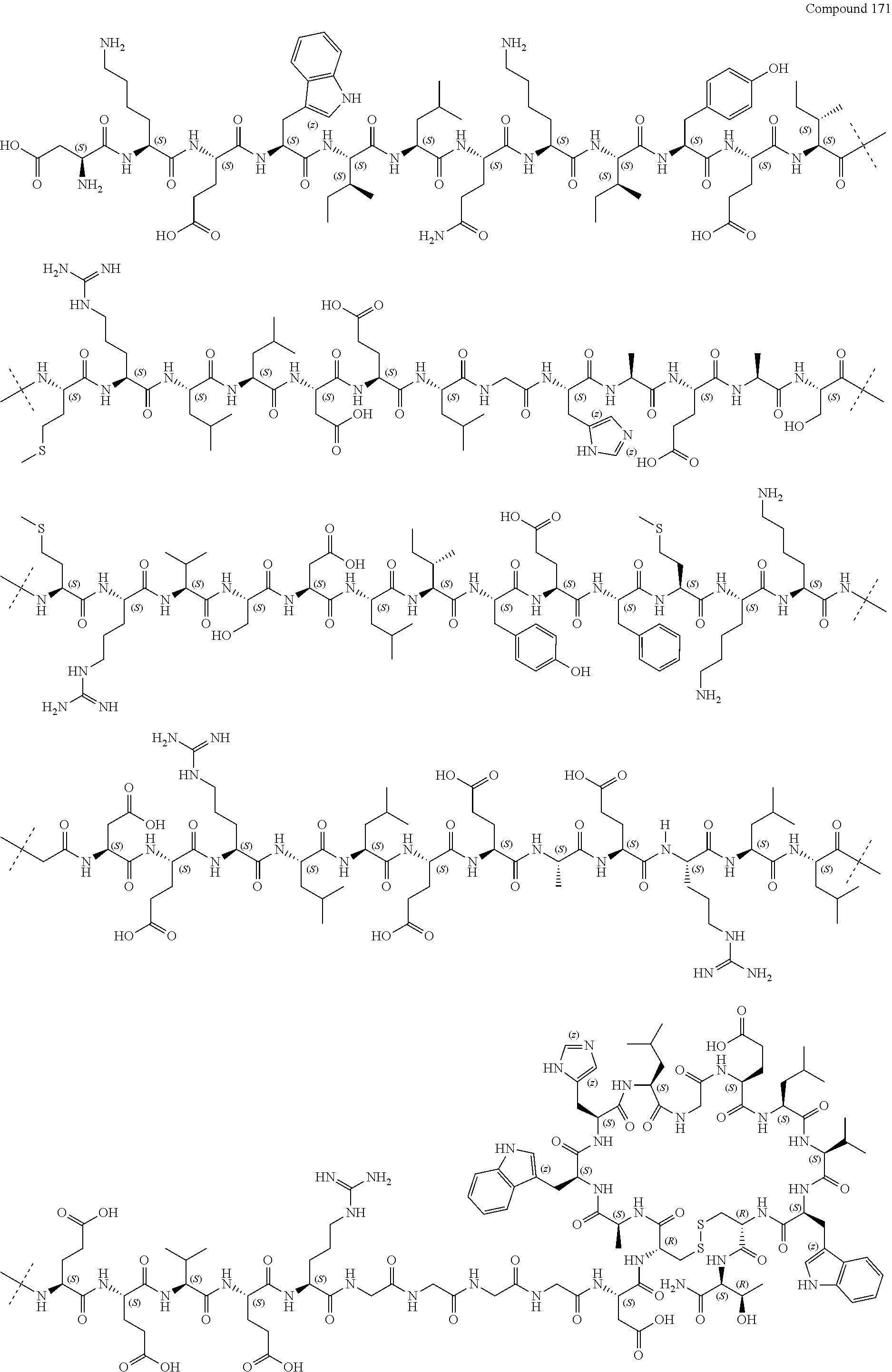

(SEQ ID NO: 65)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gly-Gly-
Gly-Asp-Cyclo[Cys-Ala-Trp-His-Leu-Gly-Glu-Leu-Val-
Trp-Cys]-Thr-NH2

1688.40, [M+6H]⁶⁺=1407.20, [M+7H]⁷⁺=1206.40, [M+8H]⁸⁺=1055.60, [M+9H]⁹⁺=938.70, [M+10H]¹⁰⁺=844.80.

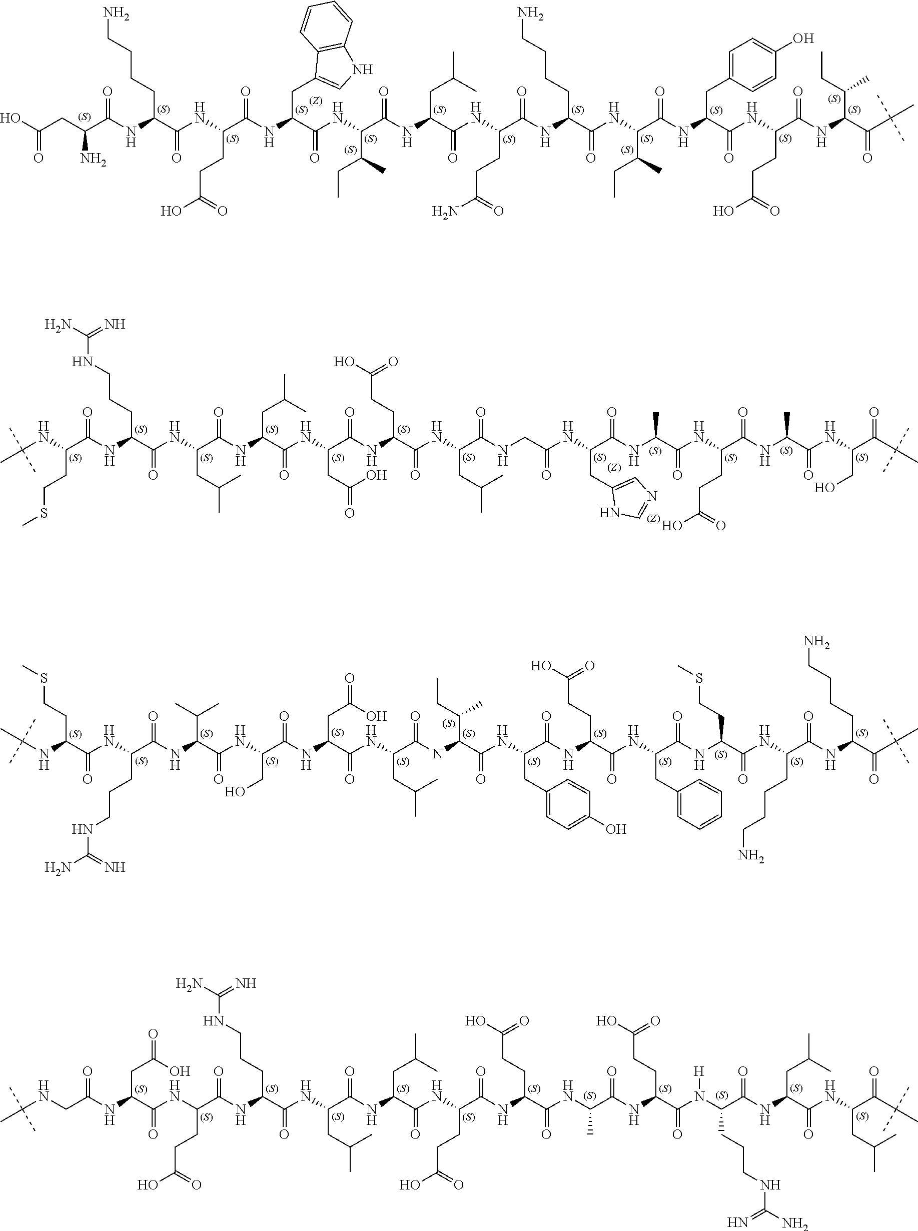

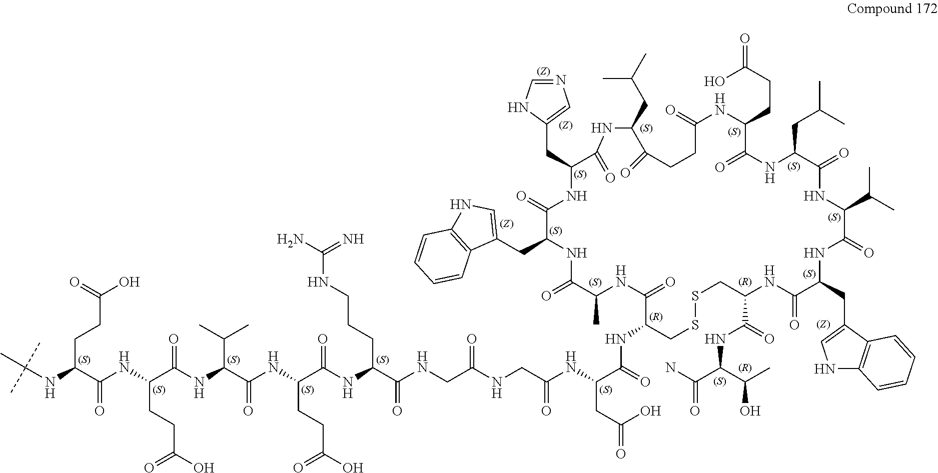

(SEQ ID NO: 66)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Gly-Gly-Asp-
Cyclo[Cys-Ala-Trp-His-Leu-Gly-Glu-Leu-Val-Trp-
Cys]-Thr-NH2

0.5 mmol resin afforded Compound 173 (41.6 mg, 80.8% purity; 0.90% yield) as a white solid. LCMS: RT=2.24 min, MS cal.: 9263.50, [M+5H]⁵⁺=1853.60, [M+6H]⁶⁺=1541.10, [M+7H]⁷⁺=1324.40, [M+8H]⁸⁺=1159.10, [M+9H]⁹⁺=1030.30, [M+10H]¹⁰=927.60, [M+11H]¹¹⁺=843.40, [M+12H]¹²⁺=772.90.

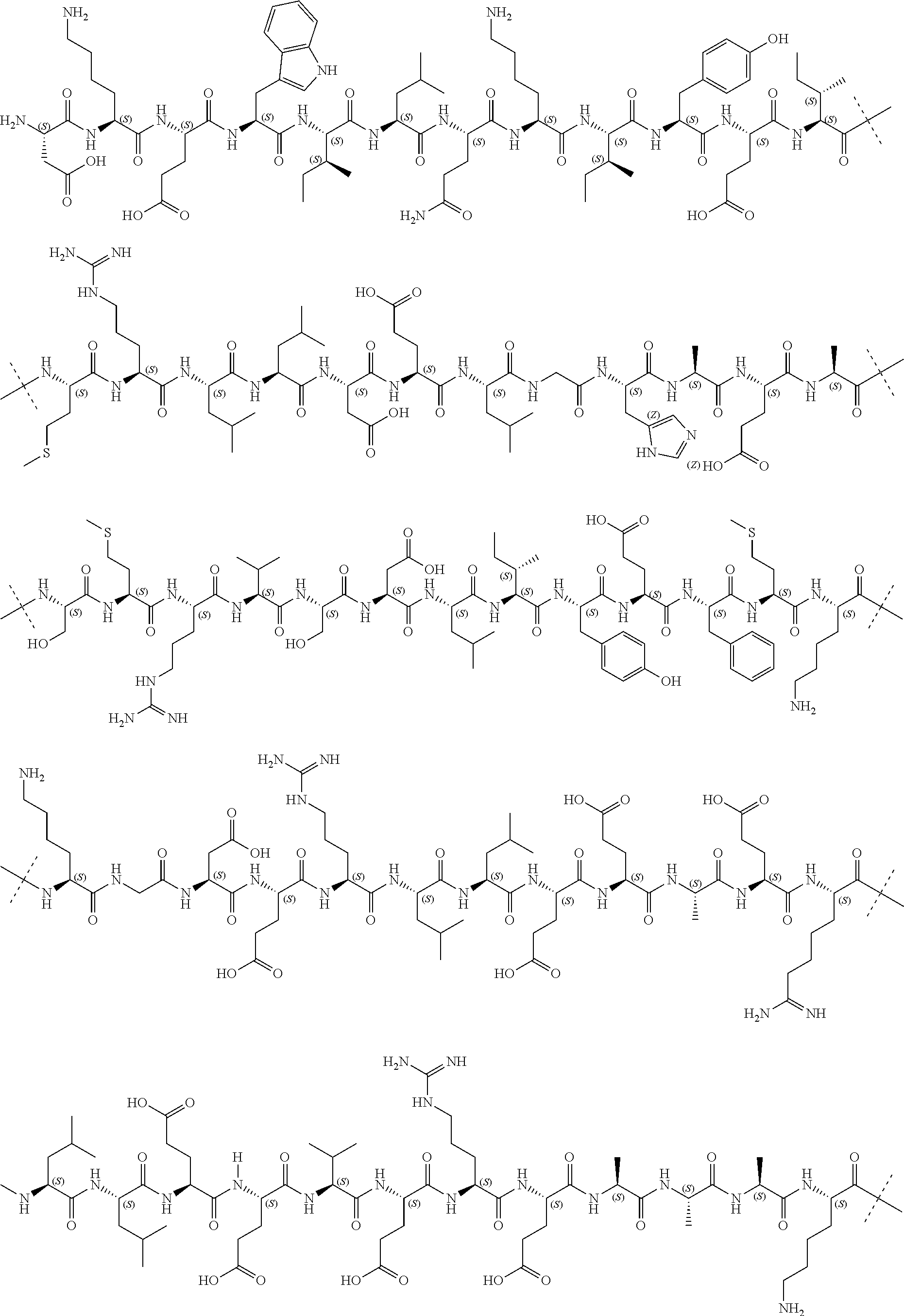

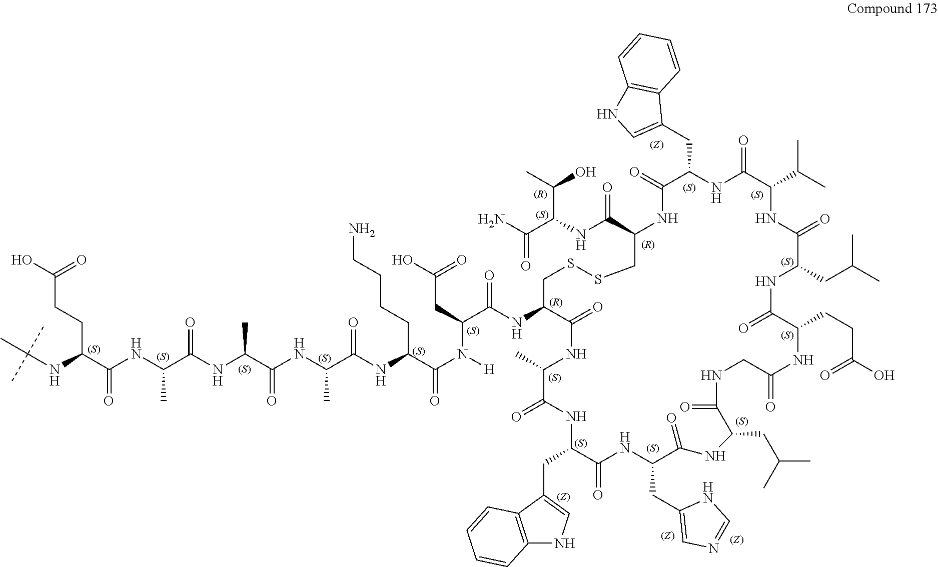

(SEQ ID NO: 67)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Ala-Ser-Met-Arg-Val-Ser-Asp-Leu-Ile-Tyr-Glu-Phe-
Met-Lys-Lys-Gly-Asp-Glu-Arg-Leu-Leu-Glu-Glu-Ala-
Glu-Arg-Leu-Leu-Glu-Glu-Val-Glu-Arg-Glu-Ala-Ala-
Ala-Lys-Glu-Ala-Ala-Ala-Lys-Asp-Cyclo[Cys-Ala-
Trp-His-Leu-Gly-Glu-Leu-Val-Trp-Cys]-Thr-NH2

0.5 mmol resin afforded Compound 174 (batch 1: 2.8 mg, 77.6% purity; batch 2: 29.7 mg, 75.9 purity; 0.72% yield) as a white solid. LCMS: RT=2.24 min, MS cal.: 9029.21, [M+5H]⁵⁺=1806.80, [M+6H]⁶⁺=1505.90, [M+7H]⁷⁺=1290.50, [M+8H]⁸⁺=1129.70, [M+9H]⁹⁺=1004.00, [M+10H]¹⁰⁺=903.80, [M+11H]¹¹⁺+=821.90.

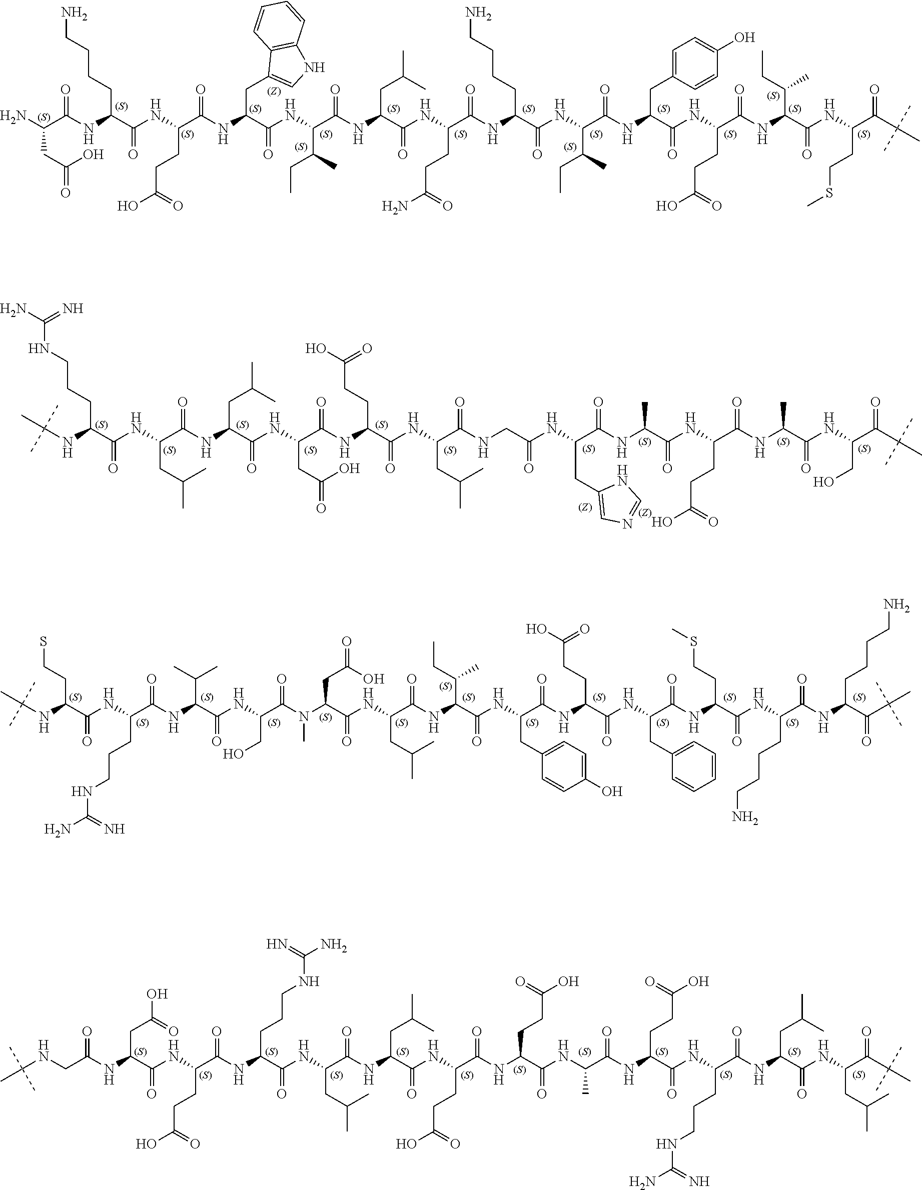

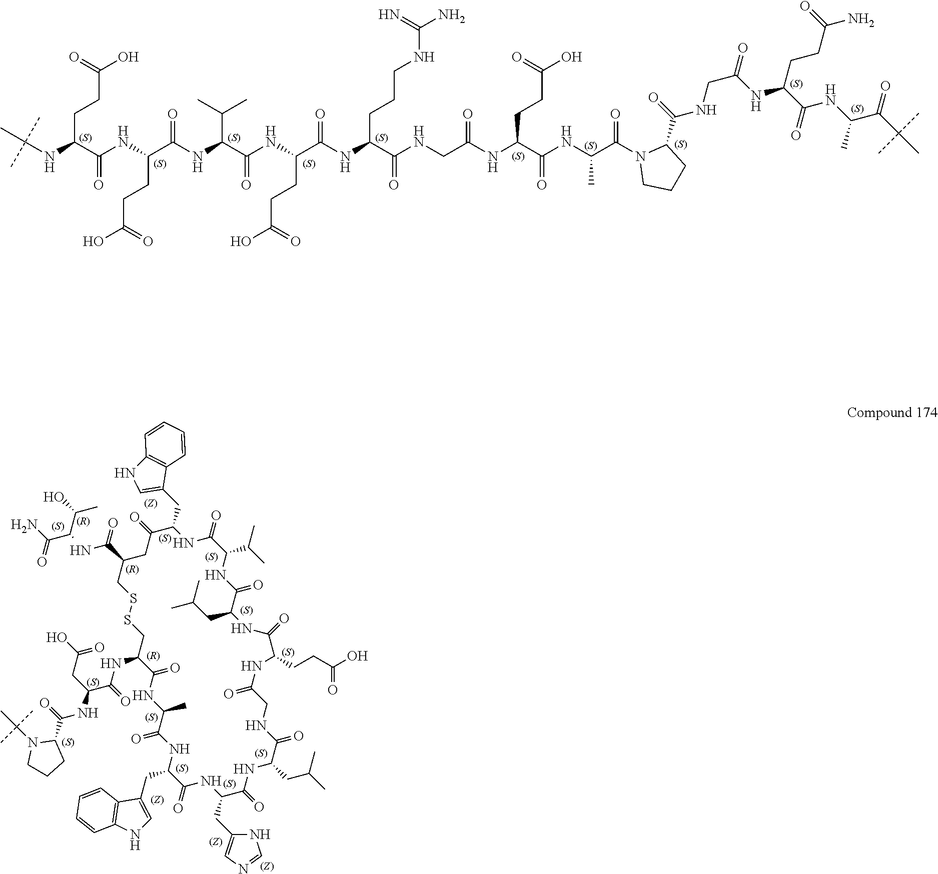

(SEQ ID NO: 68)
NH2-Asp-Lys-Glu-Trp-Ile-Leu-Gln-Lys-Ile-Tyr-Glu-
Ile-Met-Arg-Leu-Leu-Asp-Glu-Leu-Gly-His-Ala-Glu-
Glu-Ala-Ser-Met-Arg-Val-Ser-Asp-Leu-Ile-Tyr-Glu-
Phe-Met-Lys-Lys-Gly-Asp-Glu-Arg-Leu-Leu-Glu-Glu-
Ala-Glu-Arg-Leu-Leu-Glu-Glu-Val-Glu-Arg-Gly-Gln-
Ala-Pro-Gly-Gln-Ala-Pro-Asp-Cyclo[Cys-Ala-Trp-
His-Leu-Gly-Glu-Leu-Val-Trp-Cys]-Thr-NH2

### Example 16. Procedure for Preparation of Compound 175 (One Single SPPS to Form Disulfide Peptide, AM Resin)

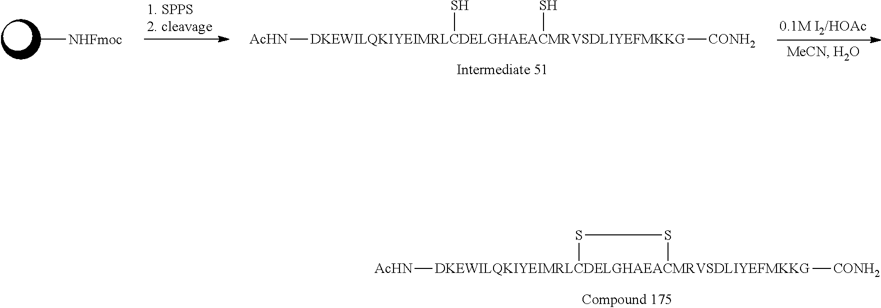

Peptide was synthesized using standard Fmoc chemistry (Rinke amide AM resin).
1) Resin preparation: To the vessel containing Rink Amide AM resin (0.200 mmol, 0.570 g, 0.350 mmol/g) and DMF (5.00 mL) for 2 h with N₂ bubbling at 20° C. Then 20% piperidine in DMF (8.00 mL) was added and the mixture was bubbled with N₂ for 30 min at 20° C. The mixture was filtered to obtain the resin. The resin was washed with DMF (8.00 mL)*5 before proceeding to next step.
2) Coupling: A solution of Fmoc-Gly-OH (0.178 g, 3.00 equiv.), HBTU (0.216 g, 2.85 equiv.) in DMF (5.00 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (8.00 mL)*5.
3) Deprotection: 20% piperidine in DMF (8.00 mL) was added to the resin and the mixture was bubbled with N₂ for 30 min at 20° C. The deprotection reaction was monitored by ninhydrin test, if it showed blue or brownish red, the reaction was completed. The resin was then washed with DMF (8.00 mL)*5.
4) Steps 2 and 3 were repeated for the following amino acids: number #2-12, in Table 13.
5) Coupling: A solution of Fmoc-Arg(Pbf)-OH (0.410 g, 3.00 equiv.), HATU (0.216 g, 2.85 equiv.) in DMF (5.00 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (8.00 mL)*5.
6) Repeat step 3 for Fmoc deprotection.
7) Steps 5 and 6 were repeated for the following amino acids: number #14-38 in Table 13.
8) Acetylation: A solution of Ac₂O/NMM/DMF (10/5/85, v/v/v, 20 mL) was added to resin (0.2 mmol, 1.23 g) and the mixture was bubbled with N₂ for 20 min. The acetylation reaction was monitored by ninhydrin test, if it showed colorless, the coupling was completed.
9) After the above steps completed, the resin was then washed with DMF (8.00 mL)*5, MeOH (8.00 mL)*5, and then dried under vacuum to afford peptide resin (AM resin, 0.200 mmol, 0.950 g).

TABLE 13
The list of amino acids and the corresponding reagents used on SPPS.

| # | Materials | Coupling reagents |
|---|---|---|
| 1 | Fmoc-Gly-OH (3.00 equiv.) | HBTU (2.85 equiv.) and DIEA (6.00 equiv.) |
| 2 | Fmoc-Lys(Boc)-OH (3.00 equiv.) | HBTU (2.85 equiv.) and DIEA (6.00 equiv.) |
| 3 | Fmoc-Lys(Boc)-OH (3.00 equiv.) | HBTU (2.85 equiv.) and DIEA (6.00 equiv.) |
| 4 | Fmoc-Met-OH (3.00 equiv.) | HBTU (2.85 equiv.) and DIEA (6.00 equiv.) |
| 5 | Fmoc-Phe-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Tyr(tBu)-OH (3.00 equiv.) | HBTU (2.85 equiv.) and DIEA (6.00 equiv.) |
| 8 | Fmoc-Ile-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-Asp(OtBu)-OH (3.00 equiv.) | HBTU (2.85 equiv.) and DIEA (6.00 equiv.) |
| 11 | Fmoc-Ser(tBu)-OH (3.00 equiv.) | HBTU (2.85 equiv.) and DIEA (6.00 equiv.) |
| 12 | Fmoc-Val-OH (3.00 equiv.) | HBTU (2.85 equiv.) and DIEA (6.00 equiv.) |
| 13 | Fmoc-Arg(Pbf)-OH (3.00 equiv.) | HATU (2.85 equiv.) and DIEA (6.00 equiv.) |
| 14 | Fmoc-Met-OH (3.00 equiv.) | HATU (2.85 equiv.) and DIEA (6.00 equiv.) |
| 15 | Fmoc-Cys(Trt)-OH (3.00 equiv.) | HATU (2.85 equiv.) and DIEA (6.00 equiv.) |
| 16 | Fmoc-Ala-OH (3.00 equiv.) | HATU (2.85 equiv.) and DIEA (6.00 equiv.) |
| 17 | Fmoc-Glu(OrBu)-OH (3.00 equiv.) | HATU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-His(Trt)-OH (3.00 equiv.) | HATU (2.85 equiv.) and DIEA (6.00 equiv.) |
| 20 | Fmoc-Gly-OH (3.00 equiv.) | HATU (2.85 equiv.) and DIEA (6.00 equiv.) |
| 21 | Fmoc-Leu-OH (3.00 equiv.) | HATU (2.85 equiv.) and DIEA (6.00 equiv.) |
| 22 | Fmoc-Glu(OtBu)-OH (3.00 equiv.) | HATU (2.85 equiv.) and DIEA (6.00 equiv.) |
| 23 | Fmoc-Asp(OtBu)-OH (3.00 equiv.) | HATU (2.85 equiv.) and DIEA (6.00 equiv.) |
| 24 | Fmoc-Cys(Trt)-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Arg(Pbf)-OH (3.00 equiv.) | HATU (2.85 equiv.) and DIEA (6.00 equiv.) |
| 27 | Fmoc-Met-OH (3.00 equiv.) | HATU (2.85 equiv.) and DIEA (6.00 equiv.) |
| 28 | Fmoc-Glu(OtBu)-OH (3.00 equiv.) | HATU (2.85 equiv.) and DIEA (6.00 equiv.) |
| 29 | Fmoc-Ile-OH (3.00 equiv.) | HATU (2.85 equiv.) and DIEA (6.00 equiv.) |
| 30 | Fmoc-Tyr(Bu)-OH (3.00 equiv.) | HATU (2.85 equiv.) and DIEA (6.00 equiv.) |
| 31 | Fmoc-Ile-OH (3.00 equiv.) | HATU (2.85 equiv.) and DIEA (6.00 equiv.) |
| 32 | Fmoc-Lys(Boc)-OH (3.00 equiv.) | HATU (2.85 equiv.) and DIEA (6.00 equiv.) |
| 33 | Fmoc-Gln(Trt)-OH (3.00 equiv.) | HATU (2.85 equiv.) and DIEA (6.00 equiv.) |
| 34 | Fmoc-Leu-OH (3.00 equiv.) | HATU (2.85 equiv.) and DIEA (6.00 equiv.) |
| 35 | Fmoc-Trp-OH (3.00 equiv.) | HATU (2.85 equiv.) and DIEA (6.00 equiv.) |
| 36 | Fmoc-Glu(OtBu)-OH (3.00 equiv.) | HATU (2.85 equiv.) and DIEA (6.00 equiv.) |
| 37 | Fmoc-Lys(Boc)-OH (3.00 equiv.) | HATU (2.85 equiv.) and DIEA (6.00 equiv.) |
| 38 | Fmoc-Asp(OtBu)-OH (3.00 equiv.) | HATU (2.85 equiv.) and DIEA (6.00 equiv.) |
| 39 | Acetylation | Ac2O/NMM/DMF (10/5/85, v/v/v, 20 mL) |

### Peptide Cleavage

1) Cleavage solution (TFA/TIS/H₂O/3-mercaptopropanoic acid, 92.5/2.5/2.5/2.5, 10.0 mL) was added to the flask containing the side chain protected peptide resin (0.200 mmol, 0.900 g) at room temperature and stirred for 2 h.
2) After filtration, the filtrate was collected.
3) The peptide was precipitated with cold isopropyl ether (50.0 mL)*2 and centrifuged (3 min at 3000 rpm).
4) The precipitate was washed with isopropyl ether for two additional times, and the crude peptide was dried under vacuum for 2 h to afford intermediate 51 (SEQ ID NO:69) (0.650 g, crude) as a white solid.

To a mixture of intermediate 51 (0.650 g, crude) in MeCN/H₂O (1/1, v/v, 200 mL) was added 0.1 M I₂/HOAc dropwise until a yellow color persisted, then the mixture was stirred at 20° C. for 5 min. The mixture was quenched by addition of 0.1 M Na₂S₂O₃ dropwise until the yellow color disappeared. After filtration, the filtrate was purified by prep-HPLC (A: 0.075% TFA/H₂O, B: MeCN) directly, followed by lyophilization to afford Compound 175 (SEQ ID NO:70) (40.9 mg, 94.0% purity, 4.05% yield, TFA salt) as a white solid. LCMS: RT=1.06 min, MS cal.: 4745.54, observed mass: [M+3H]³⁺=1582.35, [M+4H]⁴⁺+=1187.64, [M+5H]⁵⁺=949.89

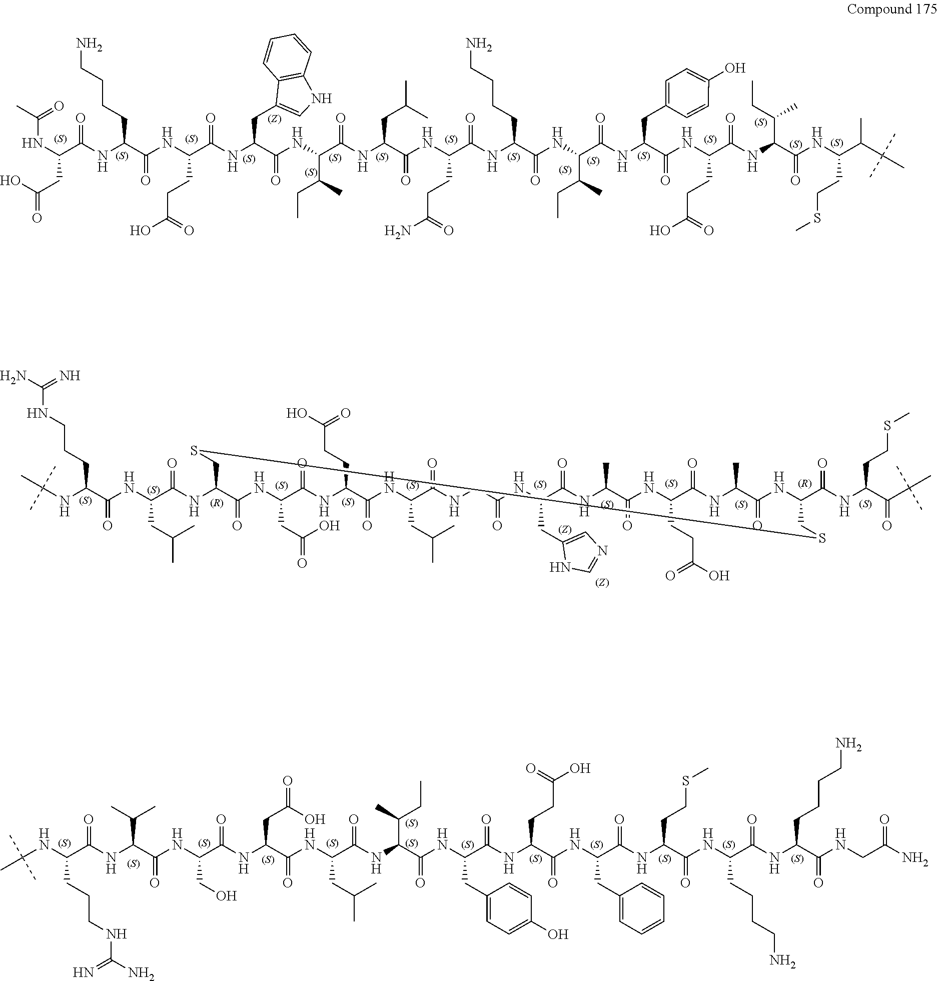

Disulfide peptides, compounds 176, 177, 178, 179, and 180 were synthesized using the same procedure as compound 138.

0.20 mmol resin afforded Compound 176 (46.0 mg, 97.2% purity, 4.62% yield, TFA salt) as a white solid. LCMS: RT=1.06 min, MS cal.: 4832.62, observed mass: [M+3H]³⁺=1612.34, [M+4H]⁴⁺=1209.54, [M+5H]⁵⁺=967.43.

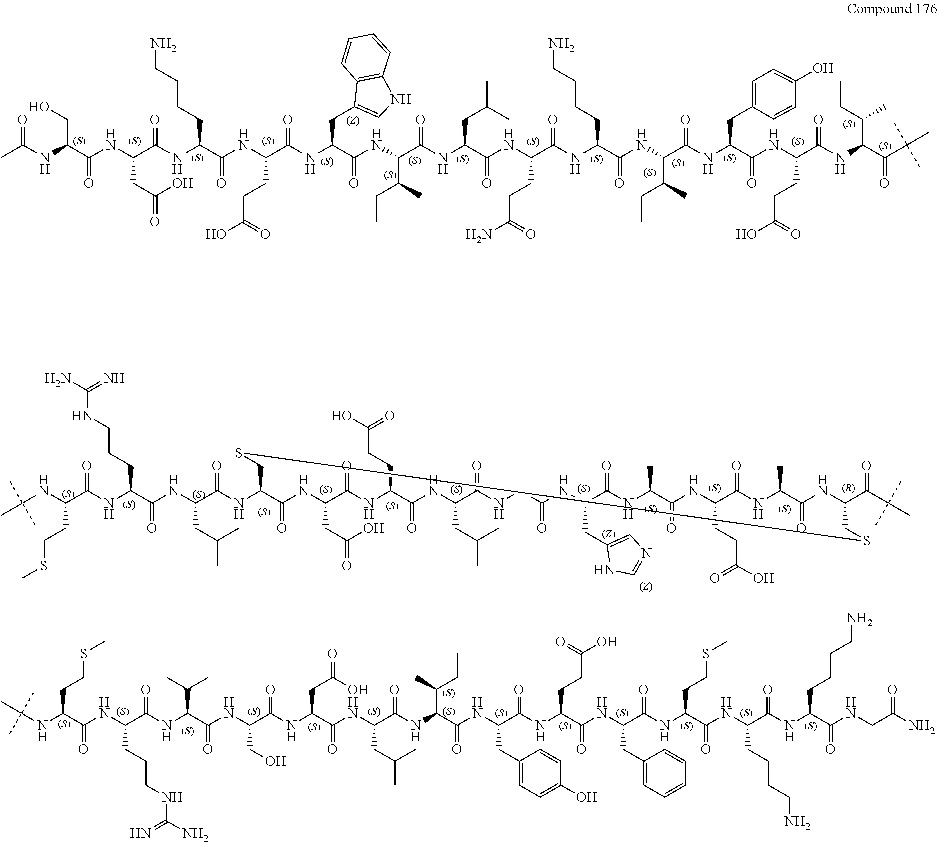

(SEQ ID NO: 71)
Ac-QKIYEIMRL-Cyclo[CDELGHAEAC]MRVSDLIYEFMKKG-NH2

0.50 mmol resin afforded Compound 177 (Batch1: 7.9 mg, 75.5% purity; batch2: 21.6 mg, 90.8% purity, batch3: 6.2 mg, 84.6% purity; batch4: 62.2 mg, 86.5% purity, AcOH salt, 5.00% yield) as a white solid. LCMS: RT=1.86 min, MS cal.: 3918.61, [M+2H]²⁺=1960.10, [M+3H]³⁺=1306.70, [M+4H]⁴⁺+=980.40, [M+5H]⁵⁺=784.90.

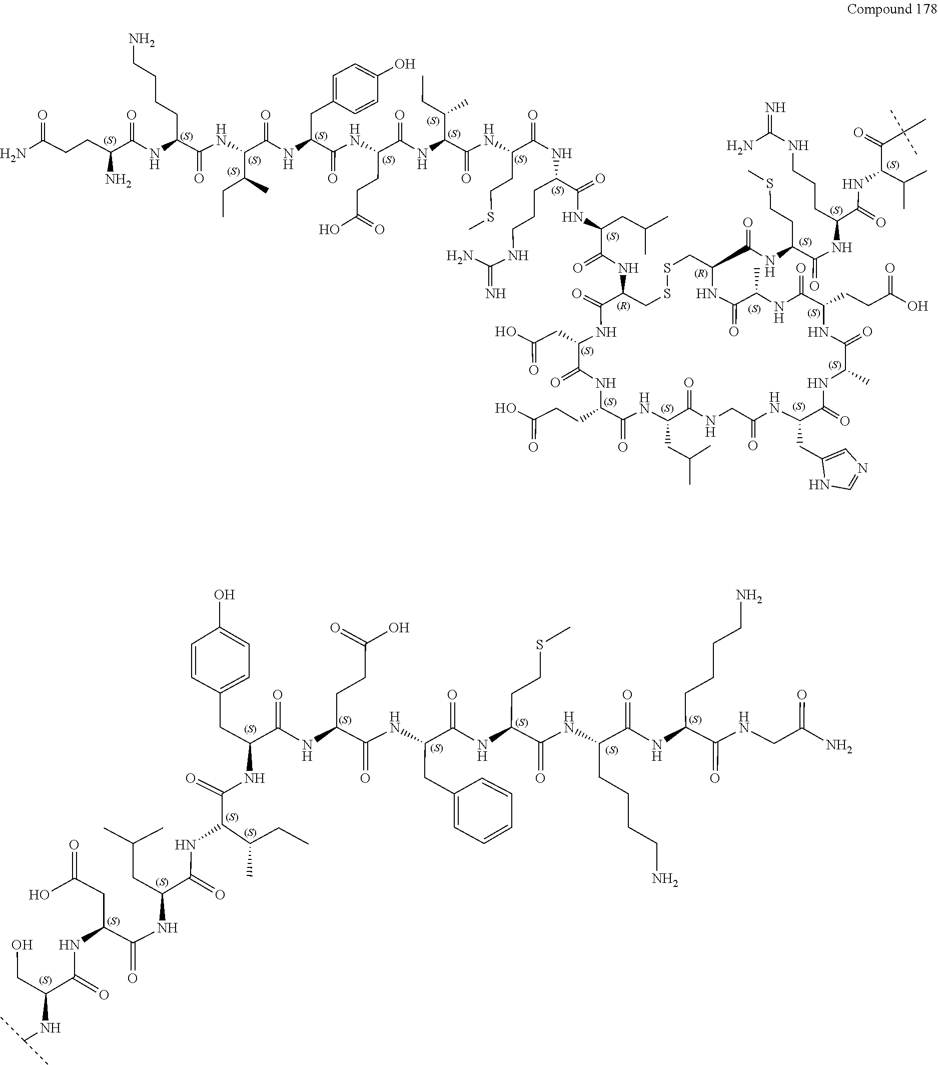

(SEQ ID NO: 72)
QKIYEIMRLCDELGHAEACMRVSDLIYEFMKKG-NH2

0.50 mmol resin afforded Compound 178 (Batch1: 35.5 mg, 91.9% purity; batch2: 38.8 mg, 86.5% purity, 3.75% yield) as a white solid. LCMS: RT=1.90 min, MS cal.: 3960.65, [M+2H]²⁺=1980.90, [M+3H]³⁺=1320.90, [M+4H]⁴⁺+=990.90, [M+5H]⁵⁺=792.80.

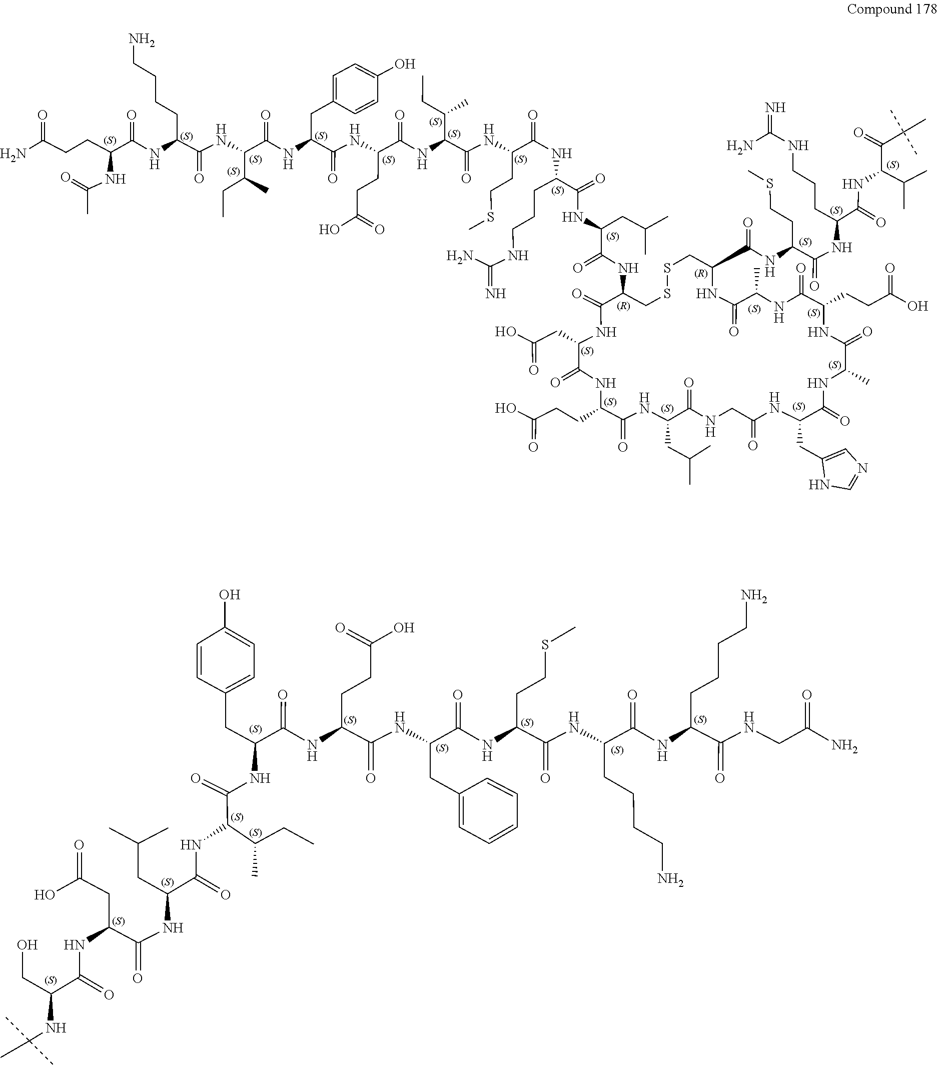

(SEQ ID NO: 73)
Ace-QKIYEIMRLCDELGHAEACMRVSDLIYEFMKKG-NH2

0.50 mmol resin afforded Compound 179 (Batch1: 18.1 mg, 84.2% purity; batch2: 22.4 mg, 96.0% purity; batch3: 44.7 mg, 95.5% purity, AcOH salt, 4.11% yield) as a white solid. LCMS: RT=1.90 min, MS cal.: 4144.92, [M+3H]³⁺=1382.30, [M+4H]⁴⁺+=1036.90, [M+5H]⁵⁺=829.70.

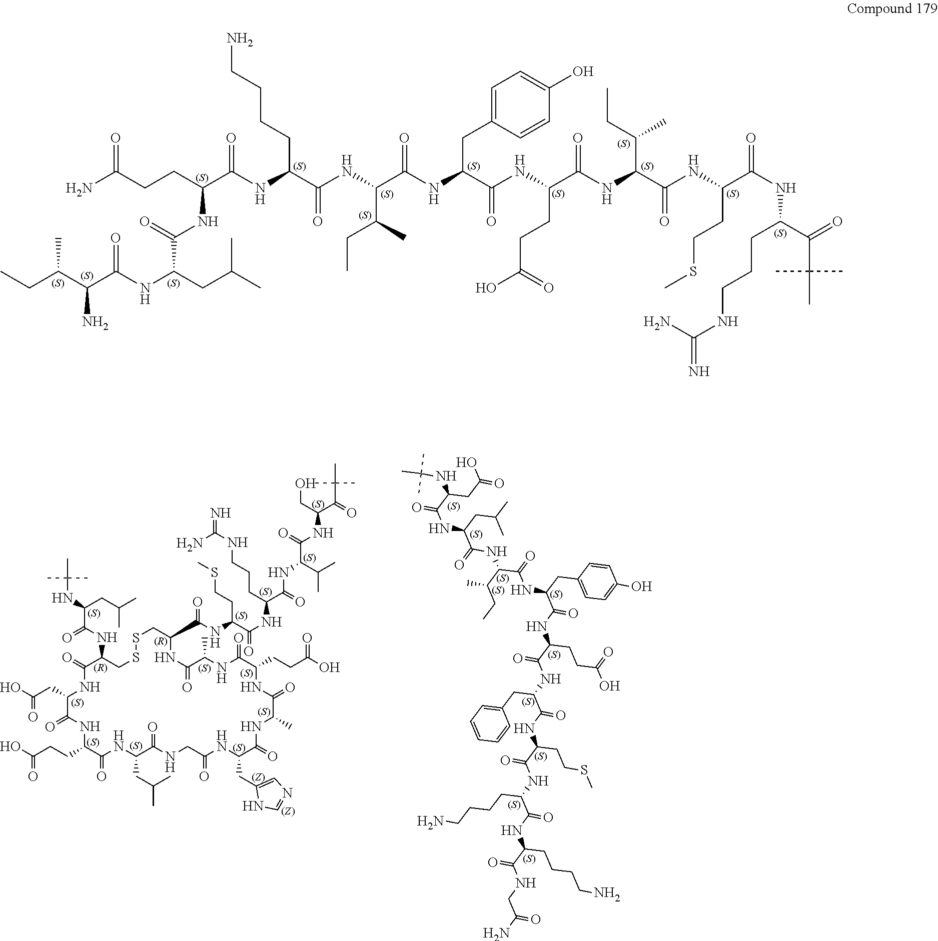

(SEQ ID NO: 74)
ILQKIYEIMRLCDELGHAEACMRVSDLIYEFMKKG-NH2

0.50 mmol resin afforded Compound 180 (Batch1: 30.4 mg, 93.2% purity; batch2: 74.5 mg, 96.2% purity, 5.01% yield) as a white solid. LCMS: RT=1.68 min, MS cal.: 4186.96, [M+3H]³⁺=1396.20, [M+4H]⁴⁺+=1047.70, [M+5H]⁵⁺=838.20.

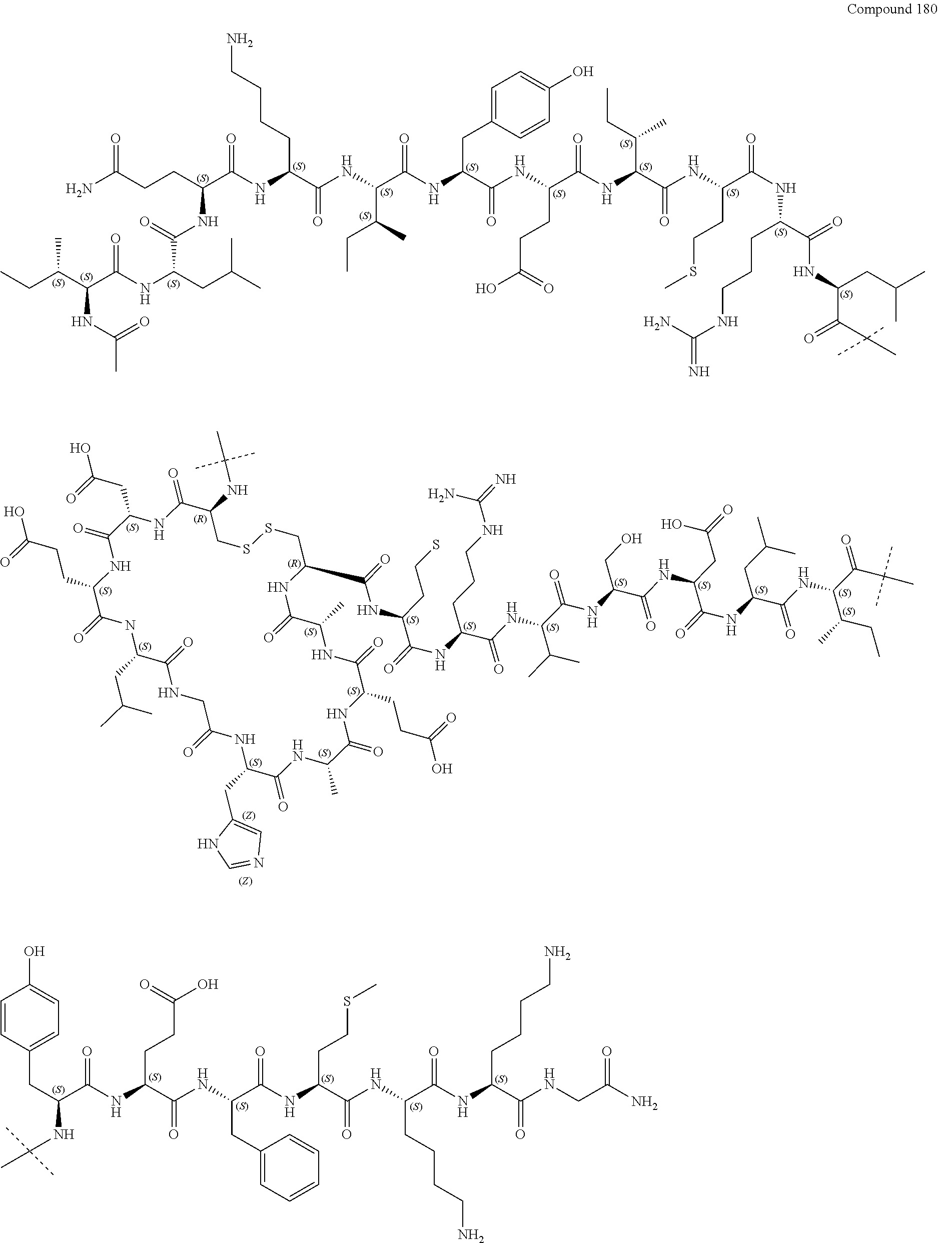

(SEQ ID NO: 75)
Ace-ILQKIYEIMRLCDELGHAEACMRVSDLIYEFMKKG-NH2

### Example 17. Procedure for Preparation of Compound 181 (One Single SPPS to Form Disulfide Peptide, CTC Resin)

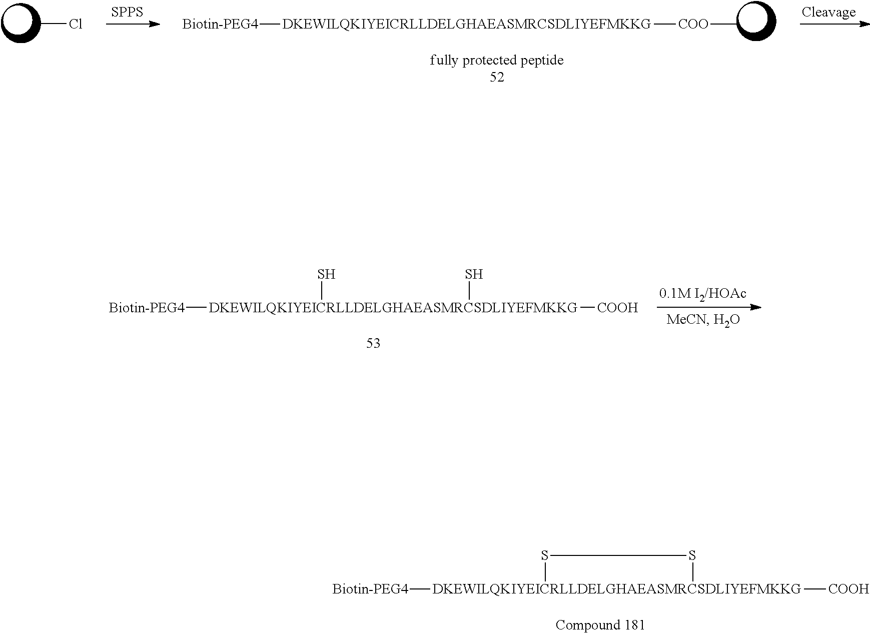

Peptide was synthesized using standard Fmoc chemistry (CTC resin).
1) Resin preparation: To the vessel containing CTC Resin (0.20 mmol, 0.202 g, 0.990 mmol/g) and Fmoc-Gly-OH (0.059 g, 0.20 mmol, 1.00 equiv.) in DCM (5 mL) was added DIEA (4.00 equiv.) dropwise and mixed for 2 h with N₂ bubbling at 20° C. Then MeOH (0.20 mL) was added and bubbled with N₂ for another 30 min. The resin was washed with DMF (8 mL)*5, followed by adding 20% piperidine in DMF (8 mL) and bubbled with N₂ for 30 min at 20° C. for Fmoc deprotection. The mixture was filtered and the resin was washed with DMF (8 mL)*5 before proceeding to next step.
2) Coupling: A solution of Fmoc-Lys(Boc)-OH (0.28 g, 3.00 equiv.), HBTU (0.21 g, 2.85 equiv.) in DMF (5 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (80 mL)*5.
3) Deprotection: 20% piperidine in DMF (8 mL) was added to the resin and the mixture was bubbled with N₂ for 30 min at 20° C. The deprotection reaction was monitored by ninhydrin test, if it showed blue or other brownish red, the reaction was completed. The resin was then washed with DMF (8 mL)*5.
4) Steps 2 and 3 were repeated for the following amino acids: number #3-12, in table 2.
5) Coupling: A solution of Fmoc-Arg(Pbf)-OH (0.39 g, 3.00 equiv.), HATU (0.21 g, 2.85 equiv.) in DMF (5 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (8 mL)*5.
6) Repeat step 3 for Fmoc deprotection.
7) Steps 5 and 6 were repeated for the following amino acids elongation: number #14-40 in Table 14.
8) After the last position completed, the resin was then washed with DMF (100 mL)*5, MeOH (100 mL)*5 and then dried under vacuum to afford peptide resin intermediate 52 (SEQ ID NO:76) (CTC resin, 0.20 mmol, 1.05 g).

TABLE 14
The list of amino acids and the corresponding reagents used on SPPS.

| # | Materials | Coupling reagents |
|---|---|---|
| 1 | Fmoc-Gly-OH (3.00 equiv.) | DIEA (4.00 equiv.) |
| 2 | Fmoc-Lys(Boc)-OH (3.00 equiv.) | HBTU (2.85 equiv.) and DIEA (6.00 equiv.) |
| 3 | Fmoc-Lys(Boc)-OH (3.00 equiv.) | HBTU (2.85 equiv.) and DIEA (6.00 equiv.) |
| 4 | Fmoc-Met-OH (3.00 equiv.) | HBTU (2.85 equiv.) and DIEA (6.00 equiv.) |
| 5 | Fmoc-Phe-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Tyr(tBu)-OH (3.00 equiv.) | HBTU (2.85 equiv.) and DIEA (6.00 equiv.) |
| 8 | Fmoc-Ile-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-Asp(OtBu)-OH (3.00 equiv.) | HBTU (2.85 equiv.) and DIEA (6.00 equiv.) |
| 11 | Fmoc-Ser(tBu)-OH (3.00 equiv.) | HBTU (2.85 equiv.) and DIEA (6.00 equiv.) |
| 12 | Fmoc-Val-OH (3.00 equiv.) | HBTU (2.85 equiv.) and DIEA (6.00 equiv.) |
| 13 | Fmoc-Arg(Pbf)-OH (3.00 equiv.) | HATU (2.85 equiv.) and DIEA (6.00 equiv.) |
| 14 | Fmoc-Met-OH (3.00 equiv.) | HATU (2.85 equiv.) and DIEA (6.00 equiv.) |
| 15 | Fmoc-Cys(Trt)-OH (3.00 equiv.) | HATU (2.85 equiv.) and DIEA (6.00 equiv.) |
| 16 | Fmoc-Ala-OH (3.00 equiv.) | HATU (2.85 equiv.) and DIEA (6.00 equiv.) |
| 17 | Fmoc-Glu(OtBu)-OH (3.00 equiv.) | HATU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-His(Trt)-OH (3.00 equiv.) | HATU (2.85 equiv.) and DIEA (6.00 equiv.) |
| 20 | Fmoc-Gly-OH (3.00 equiv.) | HATU (2.85 equiv.) and DIEA (6.00 equiv.) |
| 21 | Fmoc-Leu-OH (3.00 equiv.) | HATU (2.85 equiv.) and DIEA (6.00 equiv.) |
| 22 | Fmoc-Glu(OtBu)-OH (3.00 equiv.) | HATU (2.85 equiv.) and DIEA (6.00 equiv.) |
| 23 | Fmoc-Asp(OtBu)-OH (3.00 equiv.) | HATU (2.85 equiv.) and DIEA (6.00 equiv.) |
| 24 | Fmoc-Cys(Trt)-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Arg(Pbf)-OH (3.00 equiv.) | HATU (2.85 equiv.) and DIEA (6.00 equiv.) |
| 27 | Fmoc-Met-OH (3.00 equiv.) | HATU (2.85 equiv.) and DIEA (6.00 equiv.) |
| 28 | Fmoc-Glu(OtBu)-OH (3.00 equiv.) | HATU (2.85 equiv.) and DIEA (6.00 equiv.) |
| 29 | Fmoc-Ile-OH (3.00 equiv.) | HATU (2.85 equiv.) and DIEA (6.00 equiv.) |
| 30 | Fmoc-Tyr(tBu)-OH (3.00 equiv.) | HATU (2.85 equiv.) and DIEA (6.00 equiv.) |
| 31 | Fmoc-Ile-OH (3.00 equiv.) | HATU (2.85 equiv.) and DIEA (6.00 equiv.) |
| 32 | Fmoc-Lys(Boc)-OH (3.00 equiv.) | HATU (2.85 equiv.) and DIEA (6.00 equiv.) |
| 33 | Fmoc-Gln(Trt)-OH (3.00 equiv.) | HATU (2.85 equiv.) and DIEA (6.00 equiv.) |
| 34 | Fmoc-Leu-OH (3.00 equiv.) | HATU (2.85 equiv.) and DIEA (6.00 equiv.) |
| 35 | Fmoc-Trp-OH (3.00 equiv.) | HATU (2.85 equiv.) and DIEA (6.00 equiv.) |
| 36 | Fmoc-Glu(OtBu)-OH (3.00 equiv.) | HATU (2.85 equiv.) and DIEA (6.00 equiv.) |
| 37 | Fmoc-Lys(Boc)-OH (3.00 equiv.) | HATU (2.85 equiv.) and DIEA (6.00 equiv.) |
| 38 | Fmoc-Asp(OtBu)-OH (3.00 equiv.) | HATU (2.85 equiv.) and DIEA (6.00 equiv.) |
| 39 | Fmoc-PEG4-CH₂CH₂COOH (3.00 equiv.) | HATU (2.85 equiv.) and DIEA (6.00 equiv.) |
| 40 | Biotin-OSu (3.00 equiv.) | DIEA (6.00 equiv.) |

Peptide Cleavage: 1) Cleavage buffer (TFA/TIS/H₂O/3-Mercaptopropionic acid, 92.5/2.5/2.5/2.5, v/v/v/v, 20 mL) was added to the flask containing the side chain protected peptide resin (intermediate 52, 0.20 mmol, 1.05 g) at room temperature and stirred for 2 h.
2) After filtration, the filtrate was collected.
3) The peptide was precipitated with cold isopropyl ether (50 mL) and centrifuged (3 min at 3000 rpm).
4) Isopropyl ether washed two additional times, and the crude peptide was dried under vacuum for 2 h to afford intermediate 53 (SEQ ID NO:76) (0.86 g, crude) was obtained as a white solid.
5) To a mixture of intermediate 53 (0.86 g, crude) in MeCN/H₂O (3/7, v/v, 200 mL) was added 0.1 M I₂/AcOH dropwise until the yellow color persisted, then the mixture was stirred at 20° C. for 5 min. The mixture was quenched with 0.1 M Na₂S₂O₃ dropwise until yellow color disappeared. After filtration, the filtrate was purified by prep-HPLC (acid condition, TFA), followed by lyophilization to afford Compound 181 (SEQ ID NO:77) (16.7 mg, 92.2% purity, 1.49% yield) as a white solid. LCMS: RT=1.01 min, MS cal.: 5147.98, [M+3H]³⁺=1717.42, [M+4H]⁴⁺+=1288.14, [M+5H]⁵⁺=1030.17.

### Example 18. Procedure for Preparation of Compound 182 (Linear Peptide, AM Resin)

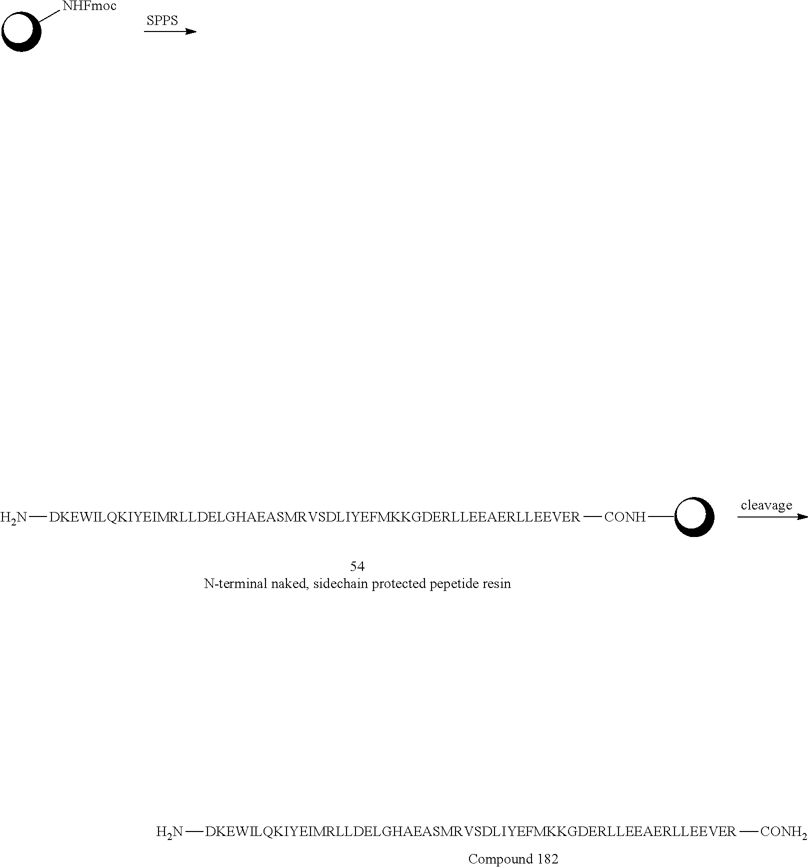

Peptide was synthesized using standard Fmoc chemistry (Rink amide AM resin).
1) Resin preparation: To the vessel containing Rink Amide AM resin (1.56 g, 0.50 mmol, 0.32 mmol/g) and DMF (20 mL) was bubbled with N₂ for 2 h at 20° C. Then 20% piperidine in DMF (40 mL) was added and the mixture was bubbled with N₂ for 30 min at 20° C. The mixture was filtered and washed with DMF (20 mL)*5 before proceeding to next step.
2) Coupling: A solution of Fmoc-Arg(Pbf)-OH (973.2 mg, 1.50 mmol, 3.00 equiv.), HBTU (541.5 mg, 5.70 mmol, 3.00 equiv.) in DMF (40 mL) was added to the resin with N₂ bubbling. Then DIEA (522.9 mL, 3.00 mmol, 6.00 equiv.) was added to the mixture dropwise and bubbled with N₂ for 30 min at 20° C. The coupling reaction was monitored by ninhydrin test. The resin was then washed with DMF (80 mL)*5.
3) Deprotection: 20% piperidine in DMF (80 mL) was added to the resin and the mixture was bubbled with N₂ for 30 min at 20° C. The deprotection reaction was monitored by ninhydrin test. The resin was then washed with DMF (80 mL)*5.
4) Steps 2 and 3 were repeated for the following amino acids elongation: Number #2-12, Table 15.
5) After all the steps were completed, the resin was washed with DMF (80 mL)*5, MeOH (80 mL)*5, then dried under reduced pressure to afford resin-bound peptide intermediate 54 (SEQ ID NO:78) (Rink AM resin, 5.20 g, 0.50 mmol).

TABLE 15
The list of amino acids and the corresponding reagents used on SPPS.

| # | Materials | Coupling reagents |
|---|---|---|
| 1 | Fmoc-Arg(Pbf)-OH (3.00 equiv.) | HBTU (2.85 equiv.) and DIEA (6.00 equiv.) |
| 2 | Fmoc-Glu(OtBu)-OH (3.00 equiv.) | HBTU (2.85 equiv.) and DIEA (6.00 equiv.) |
| 3 | Fmoc-Val-OH (3.00 equiv.) | HBTU (2.85 equiv.) and DIEA (6.00 equiv.) |
| 4 | Fmoc-Glu(OtBu)-OH (3.00 equiv.) | HBTU (2.85 equiv.) and DIEA (6.00 equiv.) |
| 5 | Fmoc-Glu(OtBu)-OH (3.00 equiv.) | HBTU (2.85 equiv.) and DIEA (6.00 equiv.) |
| 6 | Fmoc-Leu-OH (3.00 equiv.) | HBTU (2.85 equiv.) and DIEA (6.00 equiv.) |
| 7 | Fmoc-Leu-OH (3.00 equiv.) | HBTU (2.85 equiv.) and DIEA (6.00 equiv.) |
| 8 | Fmoc-Arg(Pbf)-OH (3.00 equiv.) | HBTU (2.85 equiv.) and DIEA (6.00 equiv.) |
| 9 | Fmoc-Glu(OtBu)-OH (3.00 equiv.) | HBTU (2.85 equiv.) and DIEA (6.00 equiv.) |
| 10 | Fmoc-Ala-OH (3.00 equiv.) | HBTU (2.85 equiv.) and DIEA (6.00 equiv.) |
| 11 | Fmoc-Glu(OtBu)-OH (3.00 equiv.) | HBTU (2.85 equiv.) and DIEA (6.00 equiv.) |
| 12 | Fmoc-Glu(OtBu)-OH (3.00 equiv.) | HBTU (2.85 equiv.) and DIEA (6.00 equiv.) |
| 13 | Fmoc-Leu-OH (3.00 equiv.) | HBTU (2.85 equiv.) and DIEA (6.00 equiv.) |
| 14 | Fmoc-Leu-OH (3.00 equiv.) | HBTU (2.85 equiv.) and DIEA (6.00 equiv.) |
| 15 | Fmoc-Arg(Pbf)-OH (3.00 equiv.) | HBTU (2.85 equiv.) and DIEA (6.00 equiv.) |
| 16 | Fmoc-Glu(OtBu)-OH (3.00 equiv.) | HBTU (2.85 equiv.) and DIEA (6.00 equiv.) |
| 17 | Fmoc-Asp(OtBu)-OH (3.00 equiv.) | HBTU (2.85 equiv.) and DIEA (6.00 equiv.) |
| 18 | Fmoc-Gly-OH (3.00 equiv.) | HBTU (2.85 equiv.) and DIEA (6.00 equiv.) |
| 19 | Fmoc-Lys(Boc)-OH (3.00 equiv.) | HATU (2.85 equiv.) and DIEA (6.00 equiv.) |
| 20 | Fmoc-Lys(Boc)-OH (3.00 equiv.) | HATU (2.85 equiv.) and DIEA (6.00 equiv.) |
| 21 | Fmoc-Met-OH (3.00 equiv.) | HATU (2.85 equiv.) and DIEA (6.00 equiv.) |
| 22 | Fmoc-Phe-OH (3.00 equiv.) | HATU (2.85 equiv.) and DIEA (6.00 equiv.) |
| 23 | Fmoc-Glu(OtBu)-OH (3.00 equiv.) | HATU (2.85 equiv.) and DIEA (6.00 equiv.) |
| 24 | Fmoc-Tyr(tBu)-OH (3.00 equiv.) | HATU (2.85 equiv.) and DIEA (6.00 equiv.) |
| 25 | Fmoc-Ile-OH (3.00 equiv.) | HATU (2.85 equiv.) and DIEA (6.00 equiv.) |
| 26 | Fmoc-Leu-OH (3.00 equiv.) | HATU (2.85 equiv.) and DIEA (6.00 equiv.) |
| 27 | Fmoc-Asp(OtBu)-OH (3.00 equiv.) | HATU (2.85 equiv.) and DIEA (6.00 equiv.) |
| 28 | Fmoc-Ser(tBu)-OH (3.00 equiv.) | HATU (2.85 equiv.) and DIEA (6.00 equiv.) |
| 29 | Fmoc-Val-OH (3.00 equiv.) | HATU (2.85 equiv.) and DIEA (6.00 equiv.) |
| 30 | Fmoc-Arg(Pbf)-OH (3.00 equiv.) | HATU (2.85 equiv.) and DIEA (6.00 equiv.) |
| 31 | Fmoc-Met-OH (3.00 equiv.) | HATU (2.85 equiv.) and DIEA (6.00 equiv.) |
| 32 | Fmoc-Ser(tBu)-OH (3.00 equiv.) | HATU (2.85 equiv.) and DIEA (6.00 equiv.) |
| 33 | Fmoc-Ala-OH (3.00 equiv.) | HATU (2.85 equiv.) and DIEA (6.00 equiv.) |
| 34 | Fmoc-Glu(OtBu)-OH (3.00 equiv.) | HATU (2.85 equiv.) and DIEA (6.00 equiv.) |
| 35 | Fmoc-Ala-OH (3.00 equiv.) | HATU (2.85 equiv.) and DIEA (6.00 equiv.) |
| 36 | Fmoc-His(Trt)-OH (3.00 equiv.) | HATU (2.85 equiv.) and DIEA (6.00 equiv.) |
| 37 | Fmoc-Gly-OH (3.00 equiv.) | HATU (2.85 equiv.) and DIEA (6.00 equiv.) |
| 38 | Fmoc-Leu-OH (3.00 equiv.) | HATU (2.85 equiv.) and DIEA (6.00 equiv.) |
| 39 | Fmoc-Glu(OtBu)-OH (3.00 equiv.) | HATU (2.85 equiv.) and DIEA (6.00 equiv.) |
| 40 | Fmoc-Asp(OtBu)-OH (3.00 equiv.) | HATU (2.85 equiv.) and DIEA (6.00 equiv.) |
| 41 | Fmoc-Leu-OH (3.00 equiv.) | HATU (2.85 equiv.) and DIEA (6.00 equiv.) |
| 42 | Fmoc-Leu-OH (3.00 equiv.) | HATU (2.85 equiv.) and DIEA (6.00 equiv.) |
| 43 | Fmoc-Arg(Pbf)-OH (3.00 equiv.) | HATU (2.85 equiv.) and DIEA (6.00 equiv.) |
| 44 | Fmoc-Met-OH (3.00 equiv.) | HATU (2.85 equiv.) and DIEA (6.00 equiv.) |
| 45 | Fmoc-Ile-OH (3.00 equiv.) | HATU (2.85 equiv.) and DIEA (6.00 equiv.) |
| 46 | Fmoc-Glu(OtBu)-OH (3.00 equiv.) | HATU (2.85 equiv.) and DIEA (6.00 equiv.) |
| 47 | Fmoc-Tyr(tBu)-OH (3.00 equiv.) | HATU (2.85 equiv.) and DIEA (6.00 equiv.) |
| 48 | Fmoc-Ile-OH (3.00 equiv.) | HATU (2.85 equiv.) and DIEA (6.00 equiv.) |
| 49 | Fmoc-Lys(Boc)-OH (3.00 equiv.) | HATU (2.85 equiv.) and DIEA (6.00 equiv.) |
| 50 | Fmoc-Gln(Trt)-OH (3.00 equiv.) | HATU (2.85 equiv.) and DIEA (6.00 equiv.) |
| 51 | Fmoc-Leu-OH (6.00 equiv.) | HATU (5.70 equiv.) and DIEA (12.00 equiv.) |
| 52 | Fmoc-Ile-OH (6.00 equiv.) | HATU (5.70 equiv.) and DIEA (12.00 equiv.) |
| 53 | Fmoc-Trp(Boc)-OH (6.00 equiv.) | HATU (5.70 equiv.) and DIEA (12.00 equiv.) |
| 54 | Fmoc-Glu(OtBu)-OH (6.00 equiv.) | HATU (5.70 equiv.) and DIEA (12.00 equiv.) |
| 55 | Fmoc-Lys(Boc)-OH (6.00 equiv.) | HATU (5.70 equiv.) and DIEA (12.00 equiv.) |
| 56 | Fmoc-Asp(OtBu)-OH (6.00 equiv.) | HATU (5.70 equiv.) and DIEA (12.00 equiv.) |

Peptide cleavage, cyclization and purification:
1) Cleavage solution (TFA/TIS/H₂O, 95/2.5/2.5, v/v/v, 100 mL) was added to the flask containing the side-chain protected resin-bound peptide intermediate 54 (Rink AM resin, 0.5 mmol, 5.20 g) at room temperature and stirred for 2 h.
2) After filtration, the filtrate was collected.
3) The peptide was precipitated with cold isopropyl ether (500 mL) and centrifuged (3 min at 3000 rpm).
4) Isopropyl ether washed two additional times, and the crude peptide was dried under vacuum for 2 h to afford Compound 182 (3.51 g, crude) as a white solid.
5) The crude was purified by prep-HPLC (A: 0.075% TFA/H₂O, B: MeCN), followed by lyophilization to afford Compound 182 (SEQ ID NO:78) (batch1: 44.5 mg, 91.8% purity; batch2: 23.5 mg, 90.4% purity; 2.00% yield) as a white solid. LCMS: RT=1.14 min, MS cal.: 6809.75, [M+4H]⁴⁺=1702.98, [M+5H]⁵⁺=1362.87, [M+6H]⁶⁺=1135.75, [M+7H]⁷⁺=973.76, [M+8H]⁸⁺=852.24, [M+9H]⁹⁺=757.67.

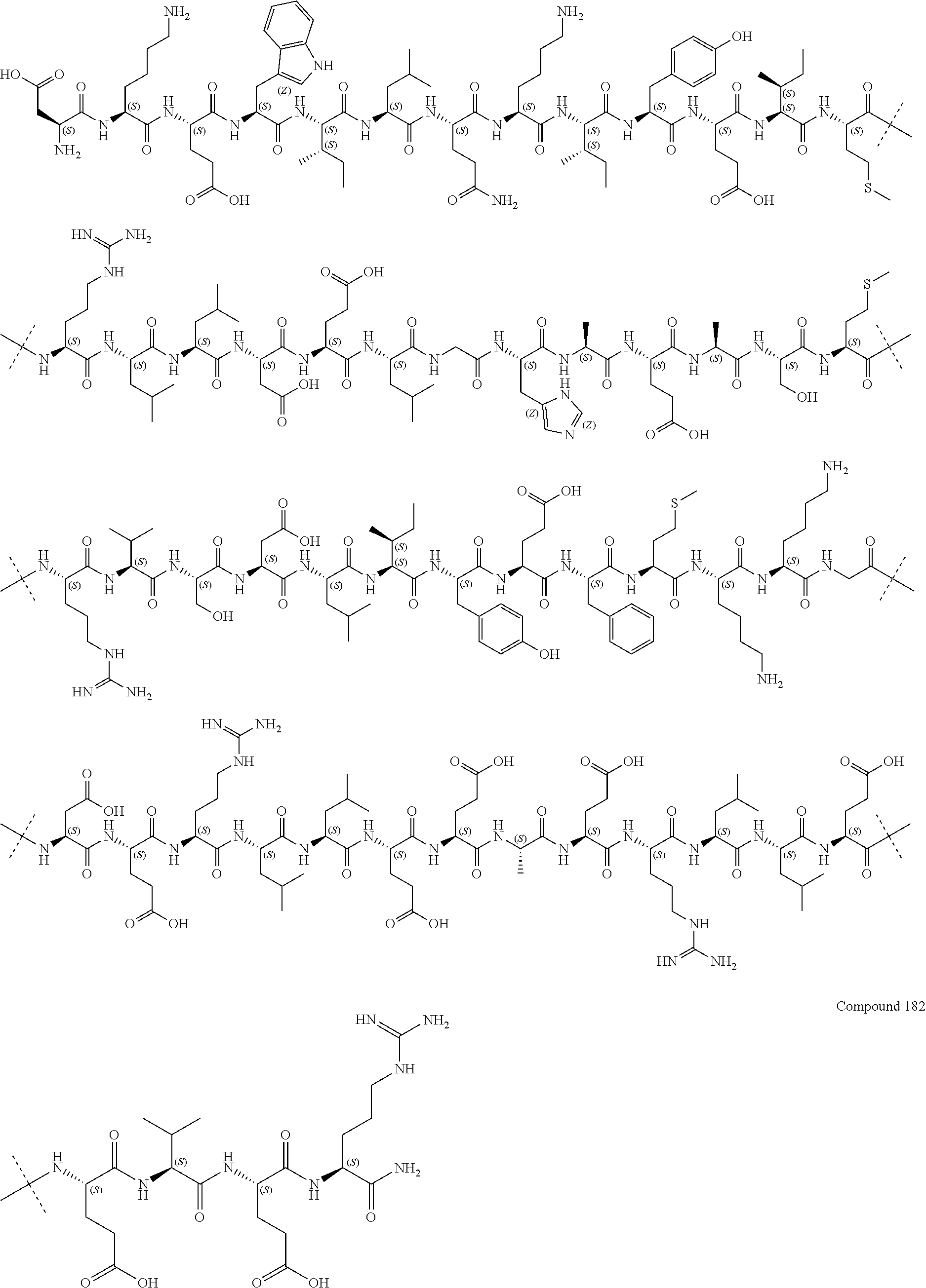

Compounds 183, 184, 185, 186, 187, 188, 189, 190, 191, 192, 193, and 194 were synthesized using the same procedure of peptide synthesis standard Fmoc chemistry, peptide cleavage and purification as Compound 182.

0.50 mmol resin afforded Compound 183 (33.5 mg, 95.1% purity, 0.92% yield) as a white solid. LCMS: RT=1.13 min, MS cal.: 7283.34, [M+4H]⁴⁺=1821.94, [M+5H]⁵⁺=1457.85, [M+6H]⁶⁺=1214.77, [M+7H]⁷⁺=1041.13, [M+9H]⁹⁺=810.53.

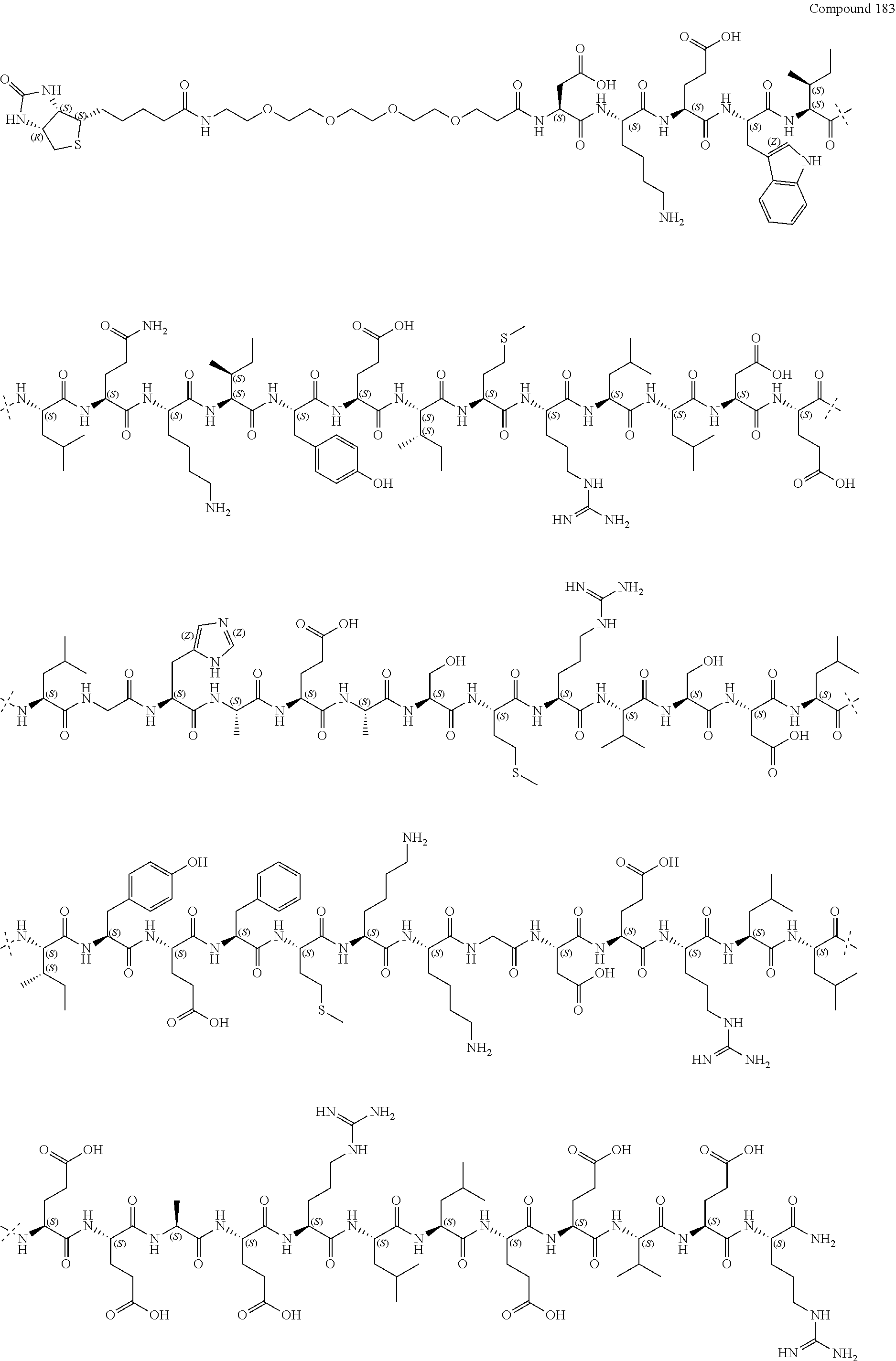

0.50 mmol resin afforded Compound 184 (71.5 mg, 88.7% purity, 1.85% yield) as a white solid. LCMS: RT=1.10 min, MS cal.: 7735.80, [M+5H]⁵⁺=1548.71, [M+6H]⁶⁺=1290.51, [M+7H]⁷⁺=1105.95, [M+8H]⁸⁺=967.82, [M+10H]¹⁰⁺=774.61.

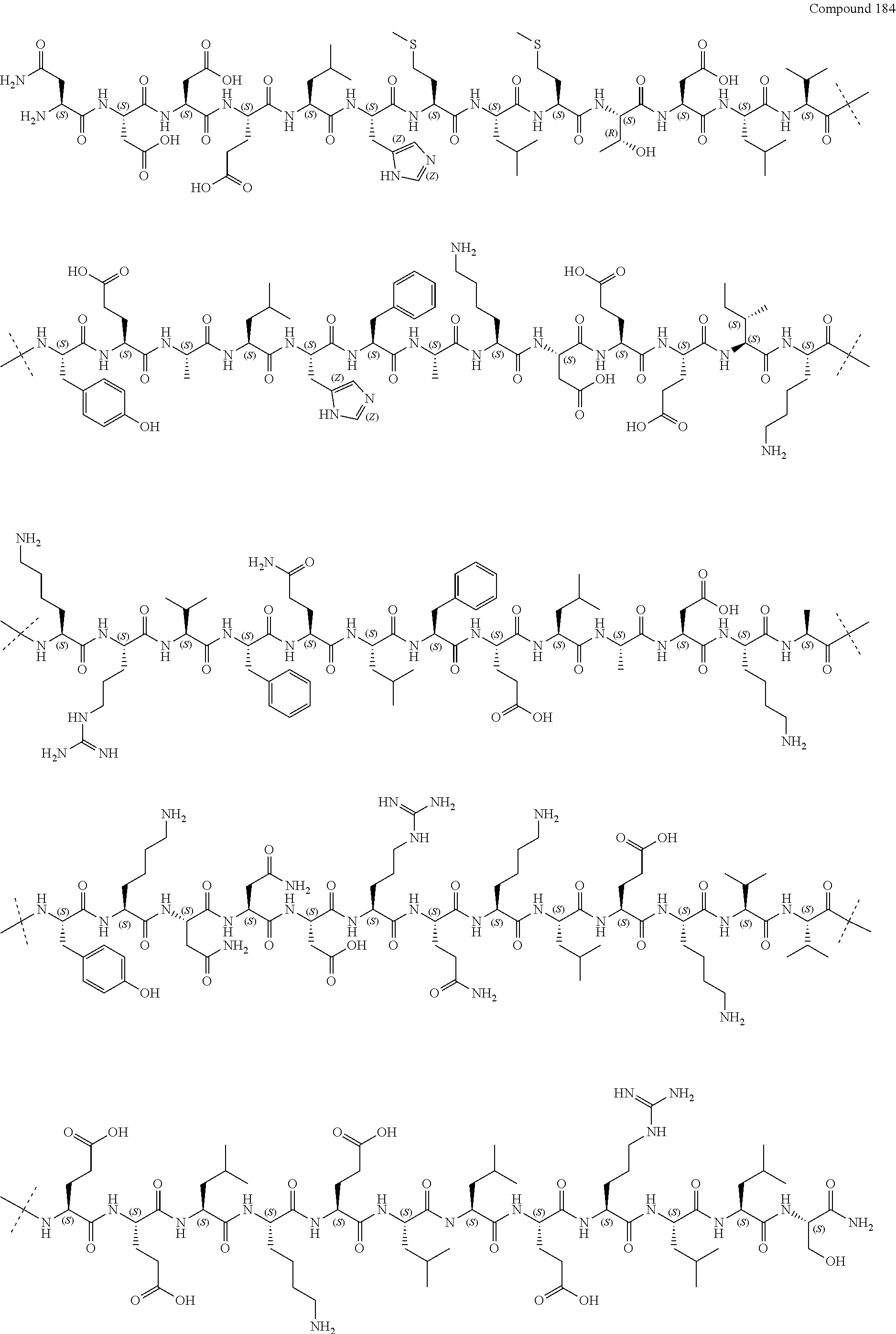

(SEQ ID NO: 79)
H2N-
NDDELHMLMTDLVYEALHFAKDEEIKKRVFQLFELADKAYKNNDRQKLEK
VVEELKELLERLLS-NH2

0.50 mmol resin afforded Compound 185 (73.2 mg, 90.3% purity, 1.78% yield) as a white solid. LCMS: RT=1.10 min, MS cal.: 8209.38, [M+6H]⁶⁺=1369.13, [M+7H]⁷⁺=1173.86, [M+8H]⁸⁺1027.34, [M+10H]¹⁰⁺=821.90.

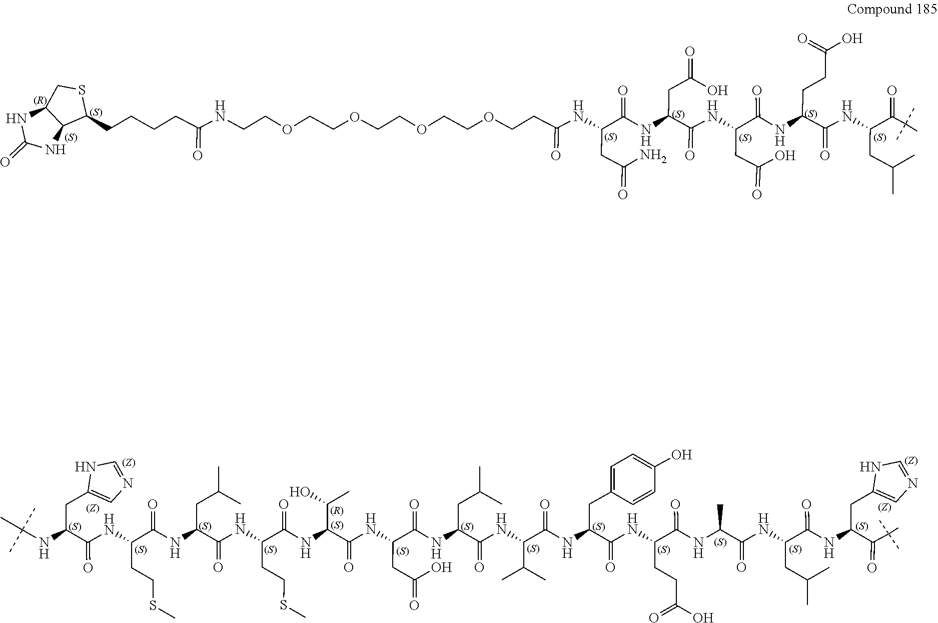

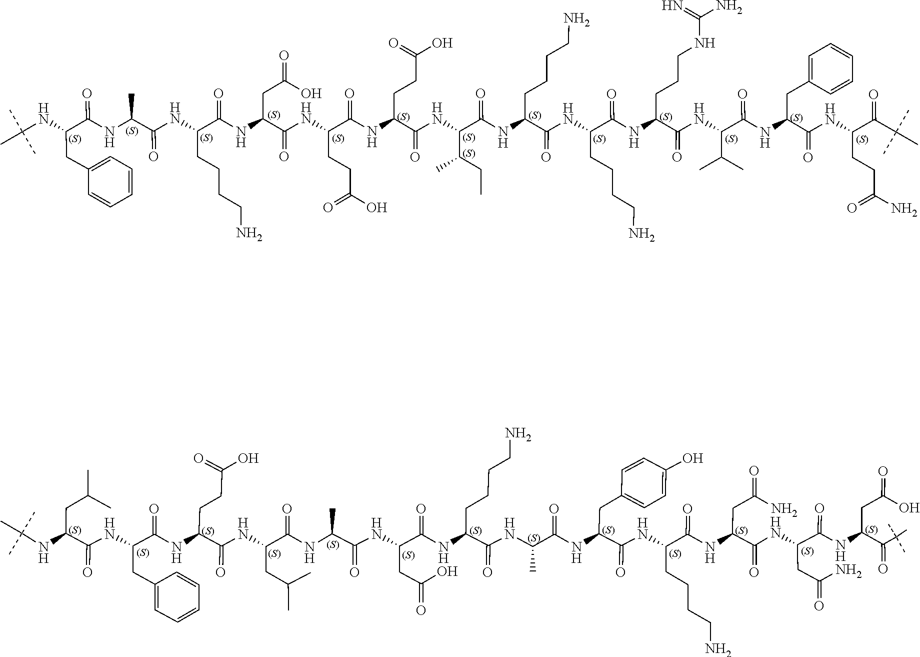

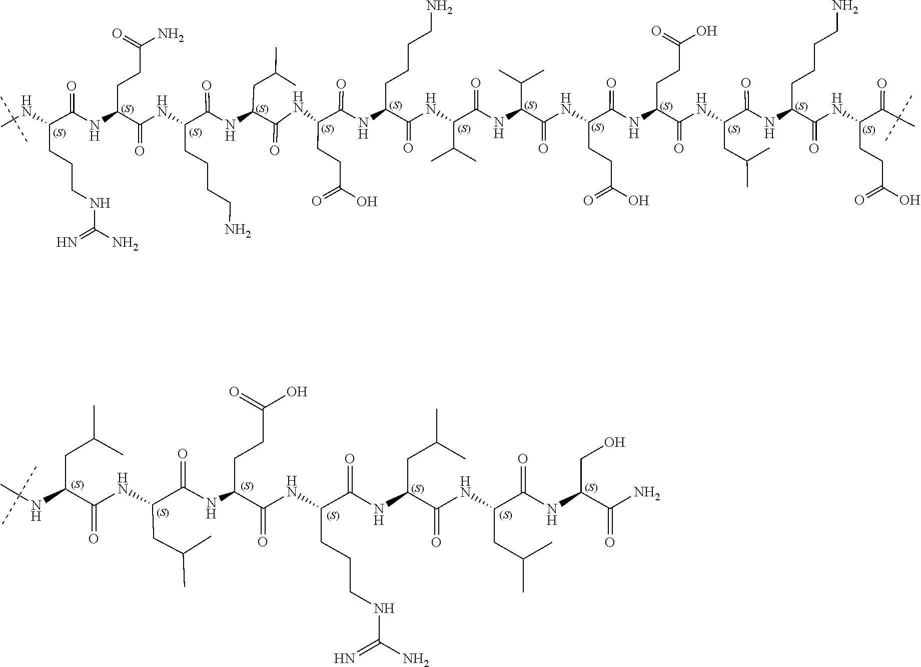

(SEQ ID NO: 80)
Biotin-PEG4-
NDDELHMLMTDLVYEALHFAKDEEIKKRVFQLFELADKAYKNNDRQKLEK
VVEELKELLERLLS-NH2

0.50 mmol resin afforded Compound 186 (25.5 mg, 88.2% purity, 0.75% yield) as a white solid. LCMS: RT=1.17 min, MS cal.: 6773.68, [M+4H]⁴⁺=1694.05, [M+5H]⁵⁺=1355.92, [M+6H]⁶⁺=1129.84, [M+7H]⁷⁺=968.44, [M+9H]⁹⁺=753.48.

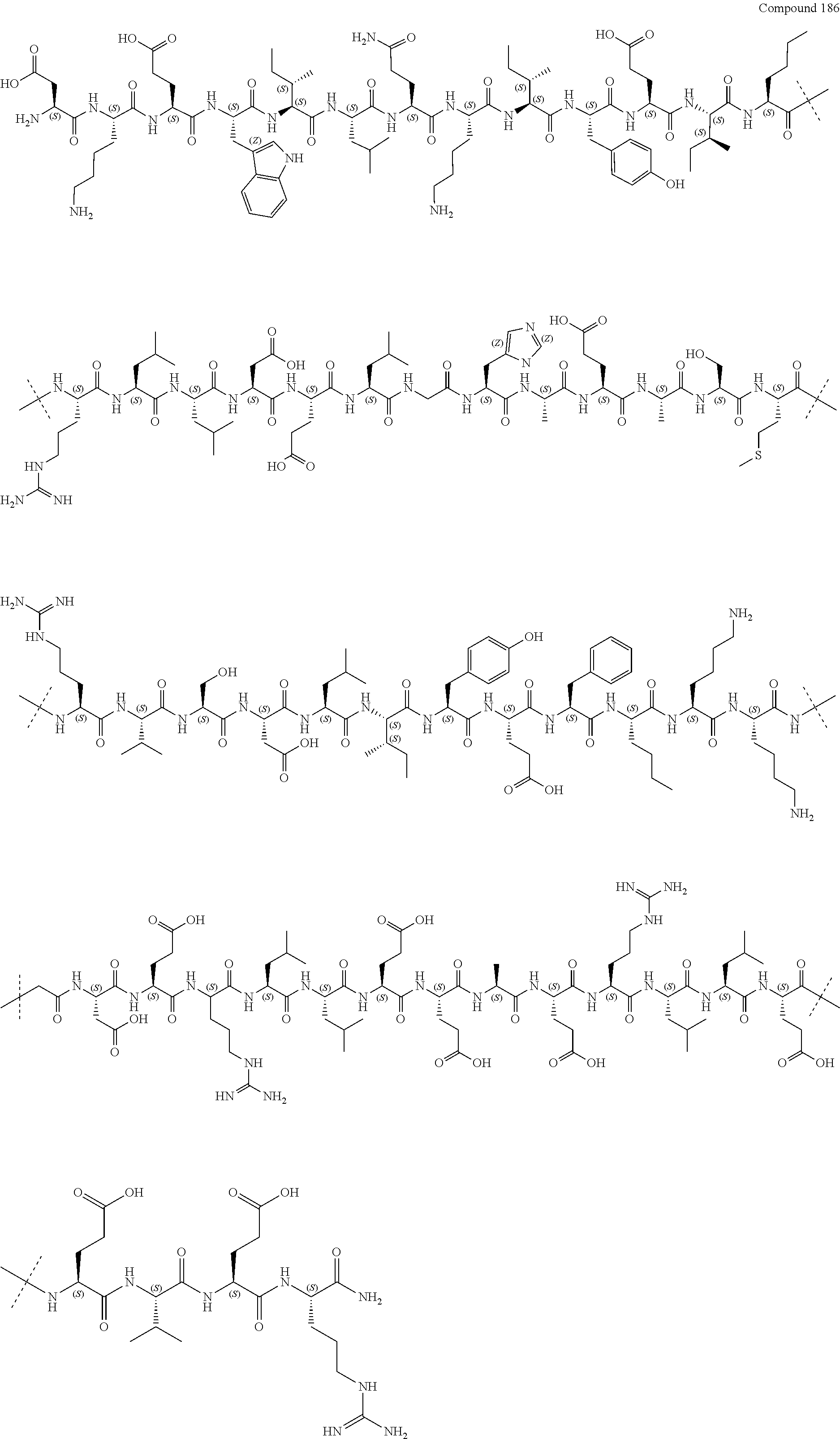

0.20 mmol resin afforded Compound 187 (14.7 mg, 90.5% purity, 0.91% yield) as a white solid. LCMS: RT=1.13 min, MS cal.: 7251.34, [M+6H]⁶⁺=1209.56, [M+7H]⁷⁺=1036.78, [M+9H]⁹⁺=806.57.

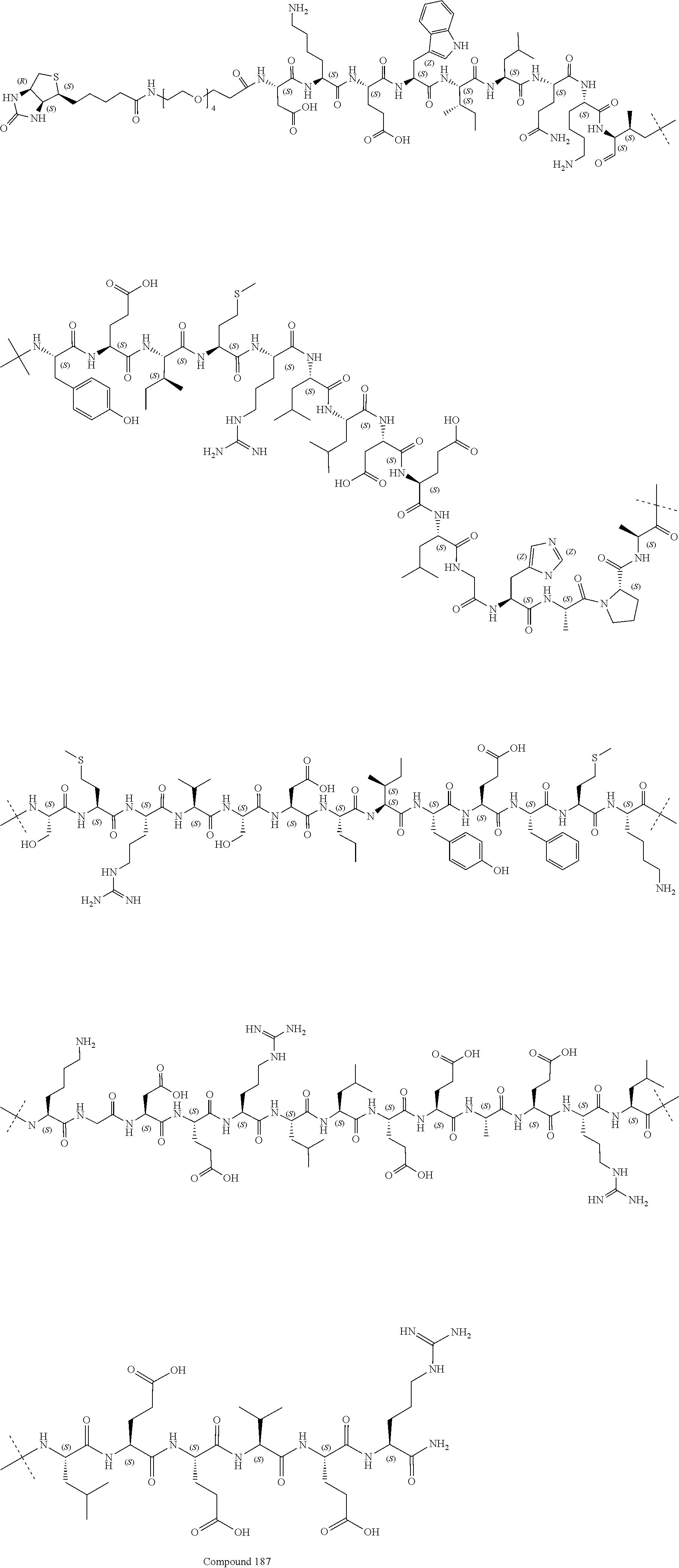

(SEQ ID NO: 81)
Biotin-PEG4-DKEWILQKIYEIMRLLDELGHAPASMRVSDLIYEFMKK
GDERLLEEAERLLEEVER-NH2

0.20 mmol resin afforded Compound 188 (15.3 mg, 87.8% purity, 0.97% yield) as a white solid. LCMS: RT=1.11 min, MS cal.: 6889.84, [M+5H]⁵⁺=1378.45, [M+6H]⁶⁺=1149.04, [M+7H]⁷⁺=985.05, [M+8H]⁸⁺=861.83, [M+9H]⁹⁺=765.97.

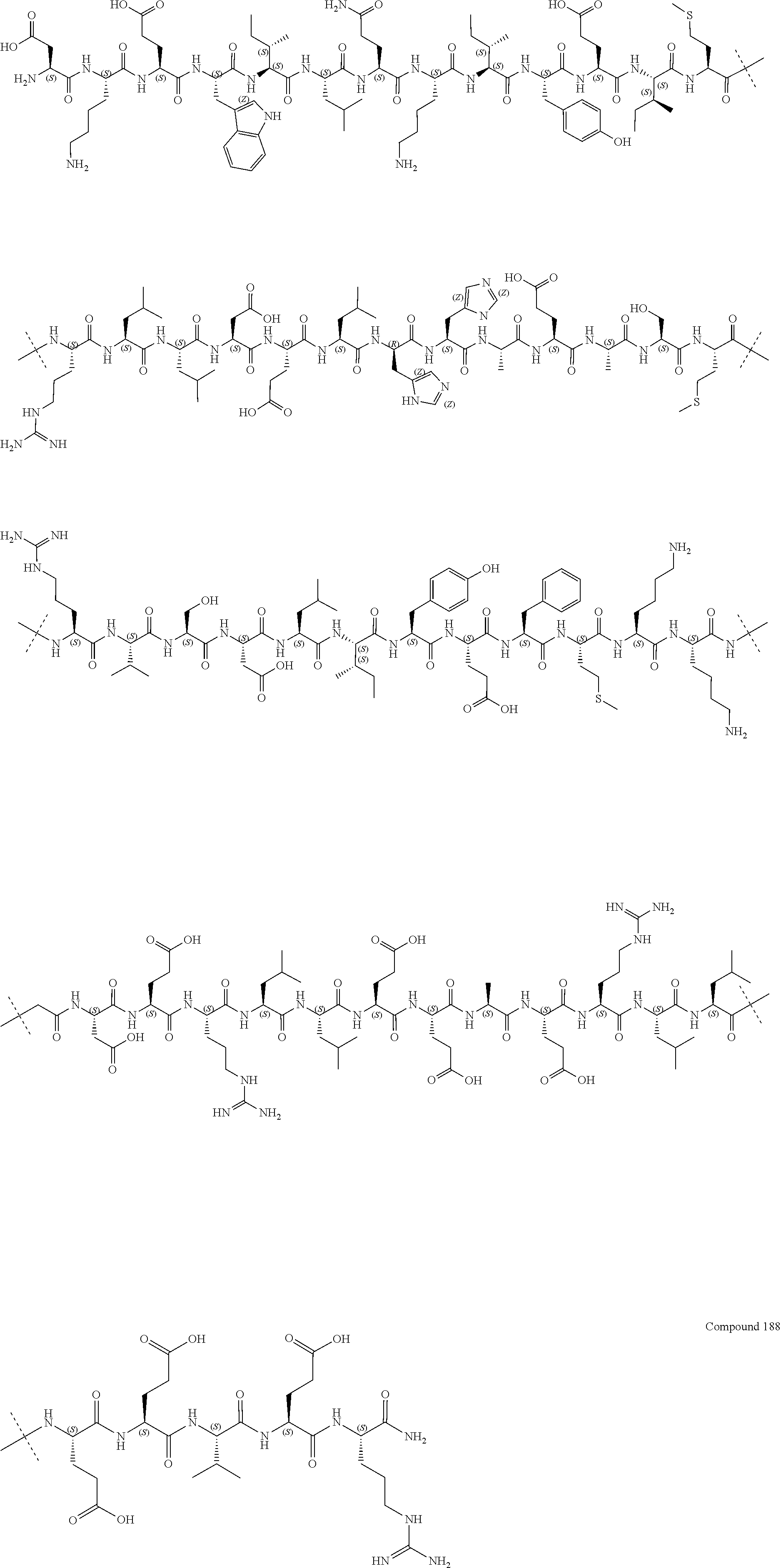

(SEQ ID NO: 82)
DKEWILQKIYEIMRLLDEL[DHis]HAPASMRVSDLIYEFMKKGDERLLE
EAERLLEEVER-NH2

0.20 mmol resin afforded Compound 189 (10.2 mg, 92.1% purity, 0.69% yield) as a white solid. LCMS: RT=1.11 min, MS cal.: 6777.76, [M+5H]⁵⁺=1356.30, [M+6H]⁶⁺=1130.24, [M+7H]⁷⁺=968.90, [M+8H]⁸⁺=847.62, [M+9H]⁹⁺=753.65.

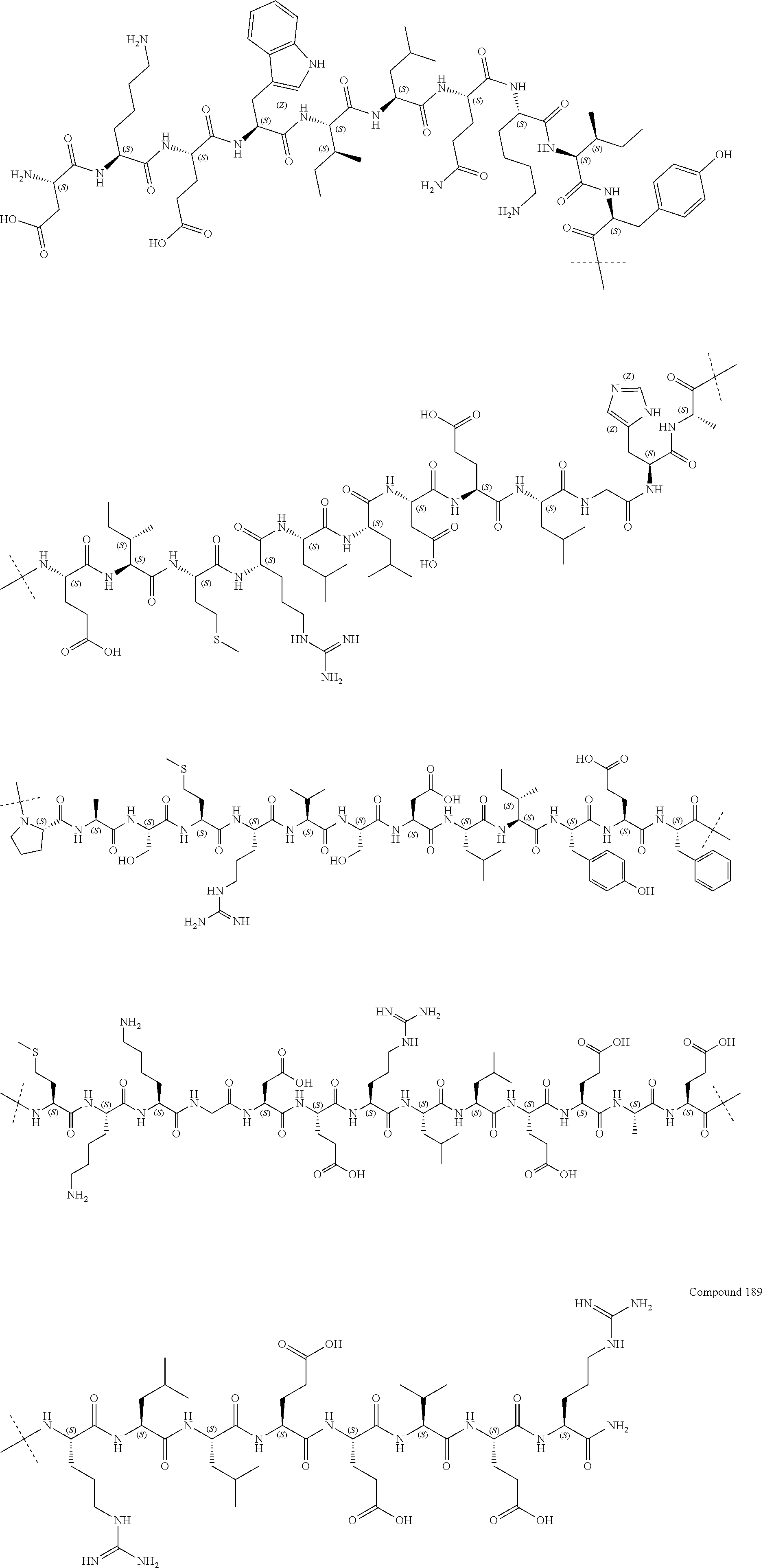

(SEQ ID NO: 83)
DKEWILQKIYEIMRLLDELGHAPASMRVSDLIYEFMKKGDERLLEEAERL
LEEVER-NH2

0.20 mmol resin afforded Compound 190 (67.1 mg, 98.3% purity, 4.71% yield) as a white solid. LCMS: RT=1.11 min, MS cal.: 6994.96, [M+5H]⁵⁺=1399.46, [M+6H]⁶⁺=1166.74, [M+7H]⁷⁺=1000.37, [M+8H]⁸⁺=875.05, [M+9H]⁹⁺=778.16.

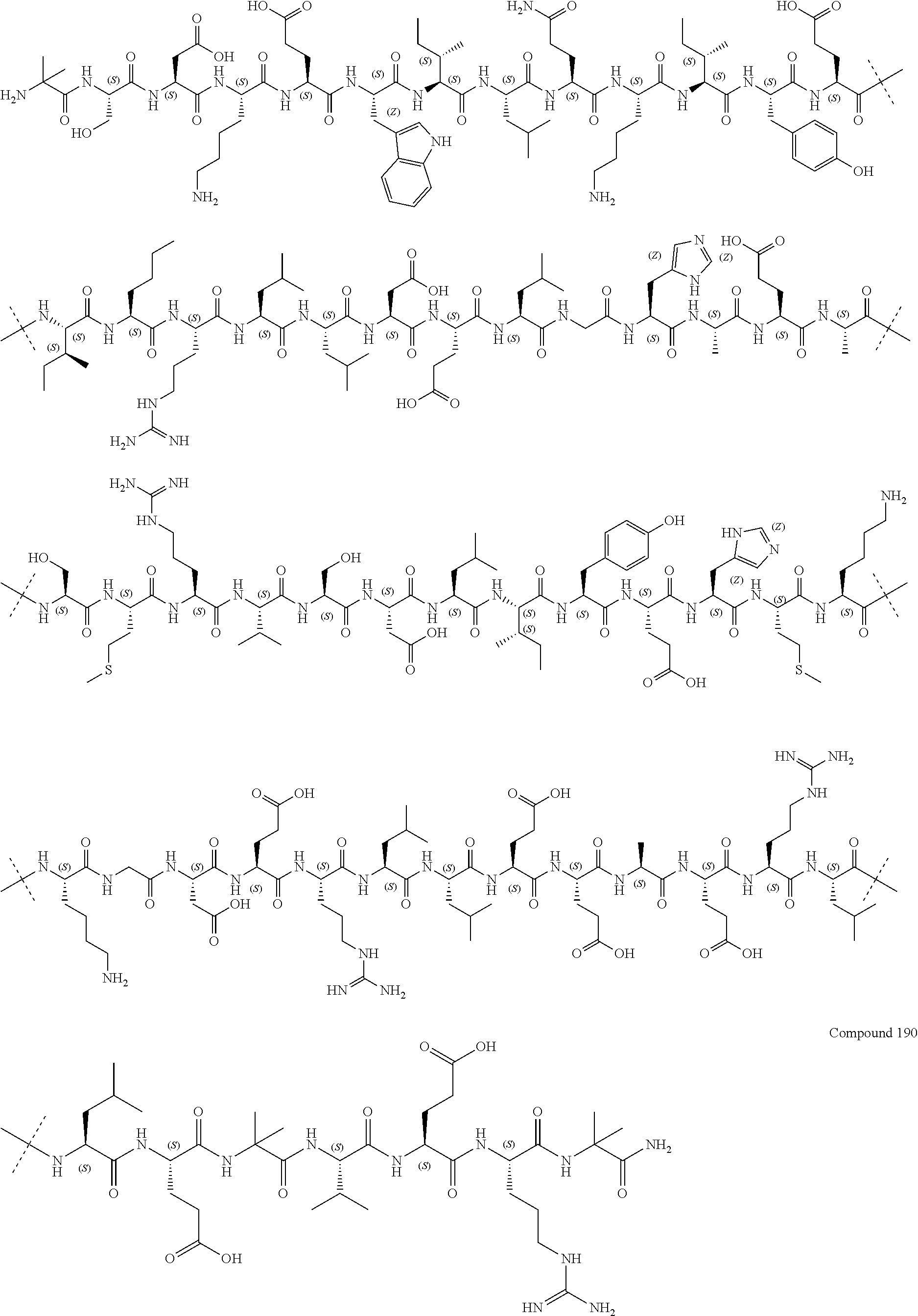

0.20 mmol resin afforded Compound 191 (17.2 mg, 92.2% purity, 4.71% yield) as a white solid. LCMS: RT=1.09 min, MS cal.: 6889.84, [M+4H]⁴⁺=1723.30, [M+5H]⁵⁺=1379.08, [M+6H]⁶⁺=1149.05, [M+7H]⁷⁺=984.51, [M+8H]⁸⁺=861.72, [M+9H]⁹⁺=766.43.

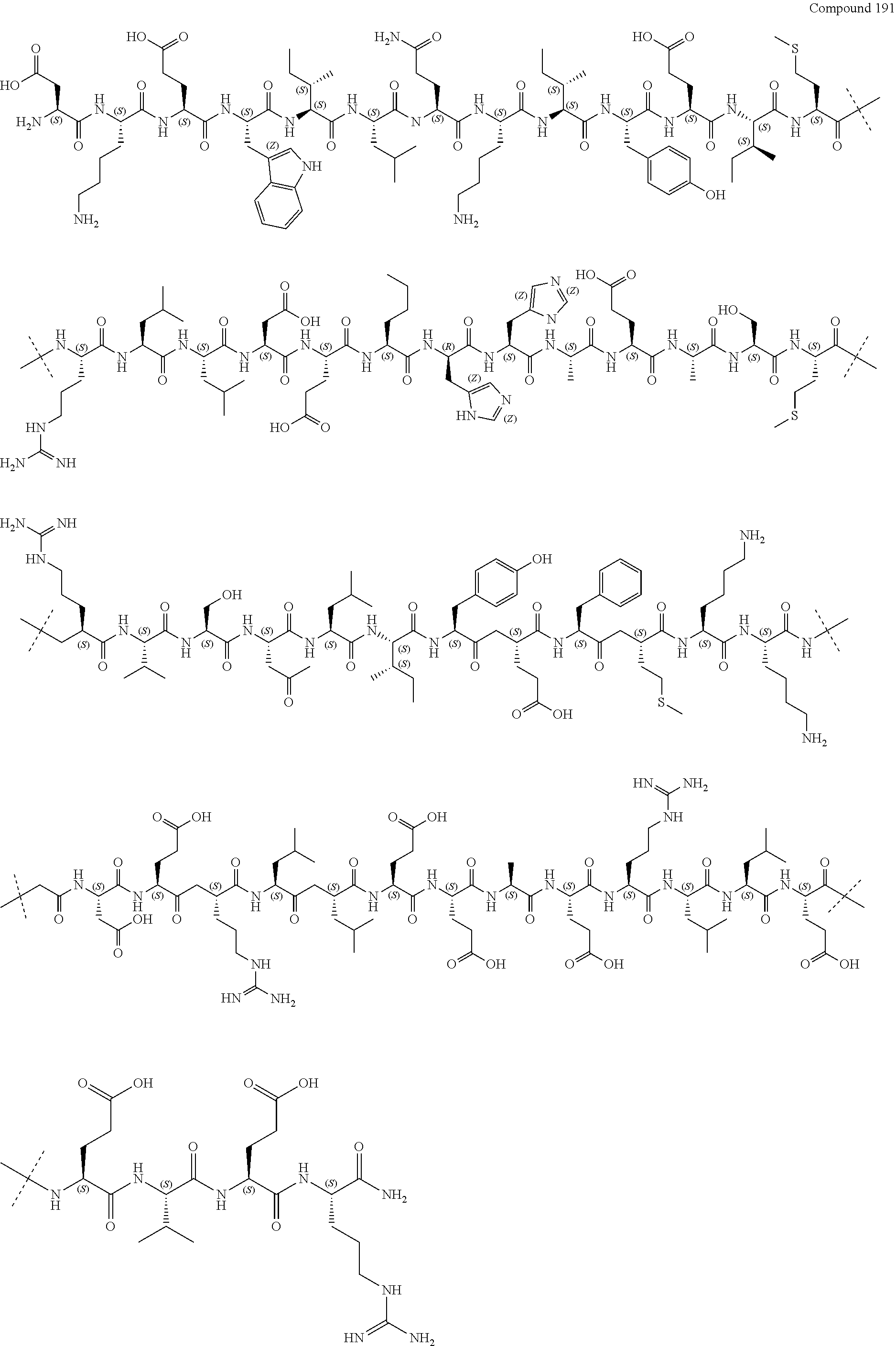

(SEQ ID NO: 84)
H2N-
DKEWILQKIYEIMRLLDE[Nle][DHis]HAEASMRVSDLIYEFMKKGDE
RLLEEAERLLEEVER-NH2

0.20 mmol resin afforded Compound 192 (43.5 mg, 95.1% purity, 4.35% yield) as a white solid. LCMS: RT=1.11 min, MS cal.: 4754.55, [M+3H]³⁺=1585.41, [M+4H]⁴⁺=1189.33, [M+5H]⁵⁺=951.72, [M+6H]⁶⁺=793.18.

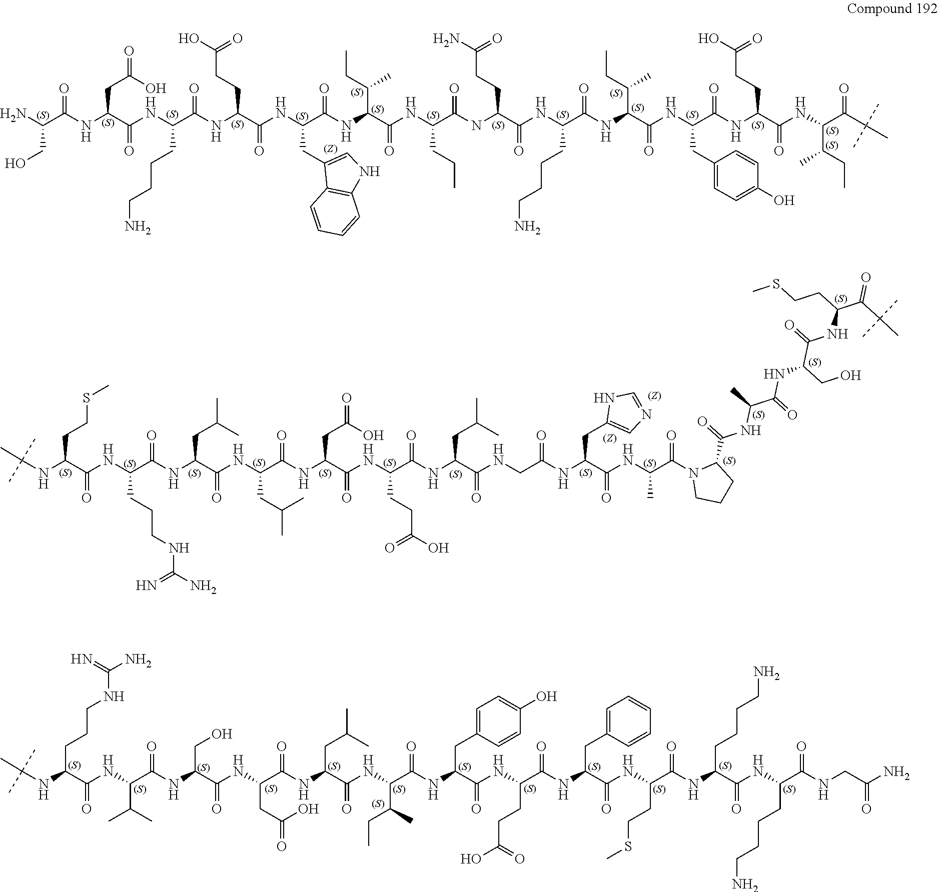

0.20 mmol resin afforded Compound 193 (43.9 mg, 96.4% purity, 4.05% yield) as a white solid. LCMS: RT=1.11 min, MS cal.: 5228.13, [M+3H]³⁺=1743.50, [M+4H]⁴⁺=1307.67, [M+5H]⁵⁺=1046.45, [M+6H]⁶⁺=872.30.

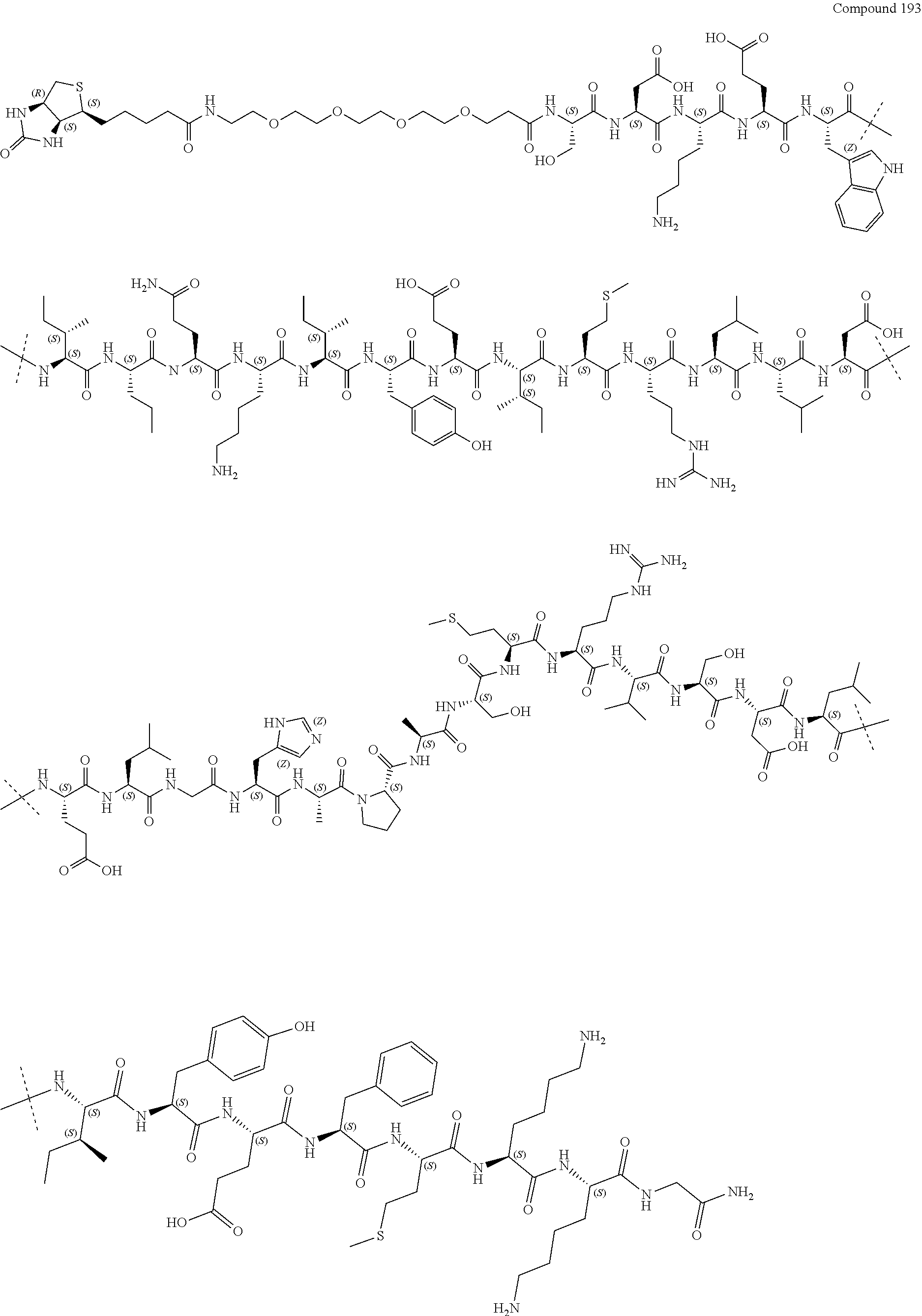

0.20 mmol resin afforded Compound 194 (43.9 mg, 96.4s purity, 4.05% yield) as a white solid. LCMS: RT=1.013 min, MS cal.: 4699.47, [M+3H]³⁺=1567.30, [M+4H]⁴⁺=1175.59, [M+5H]⁵⁺=940.60, [M+6H]⁶⁺=784.02.

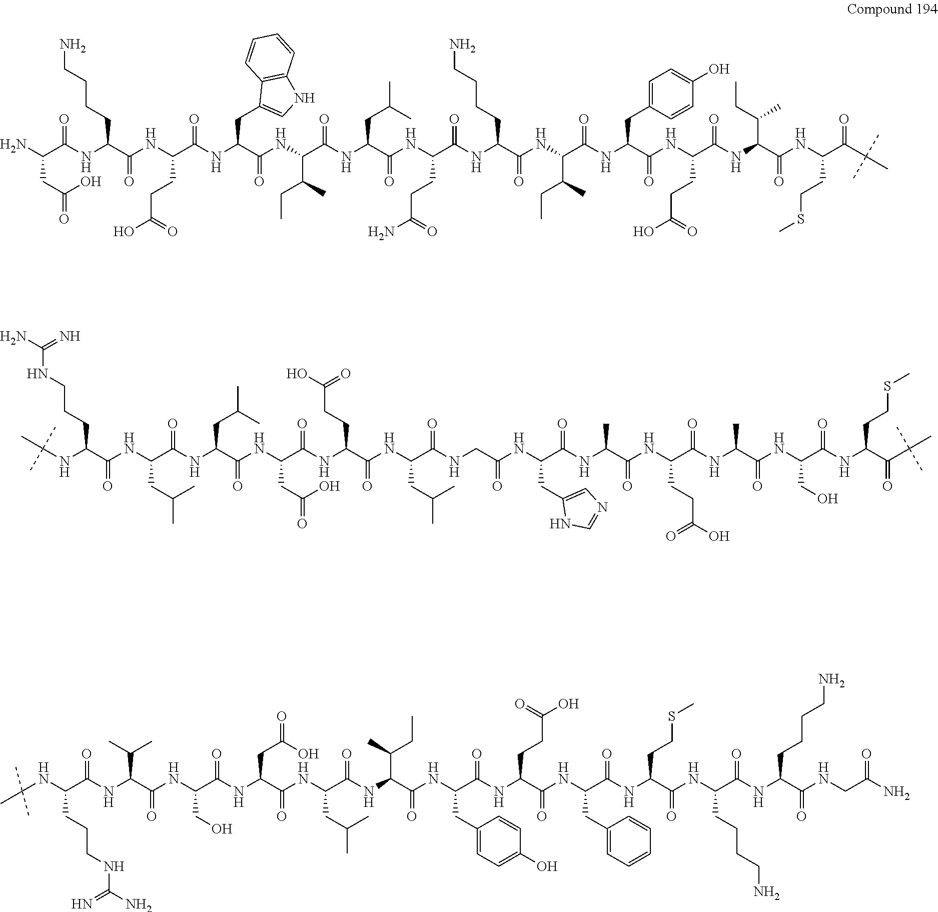

### Example 19. Procedure for Preparation of Compound 105 (Linear Peptide with Ac)

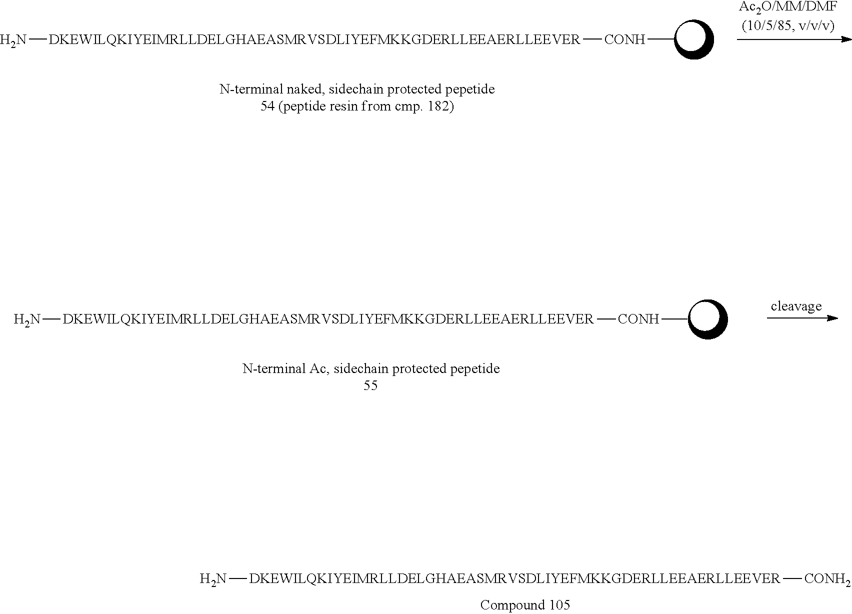

Peptide was synthesized using standard Fmoc chemistry, and SPPS was identical as in Compound 182.

### Preparation of Intermediate 55 (Acetylation).

Acetylation: A solution of Ac₂O/NMM/DMF (10/5/85, v/v/v, 30 mL) was added to resin (intermediate 54, SEQ ID NO:85, 0.5 mmol, 5.0 g) and the mixture was bubbled with N₂ for 20 min. The acetylation reaction was monitored by ninhydrin test, if it showed colorless, the coupling was completed. The resin was then washed with DMF (60 mL)*5, MeOH (20 mL)*5, and then dried under vacuum to afford peptide resin Intermediate 55 (SEQ ID NO:86) (AM resin, 0.5 mmol, 5.0 g). Peptide Cleavage and Purification:
1) Cleavage buffer (TFA/TIS/H₂O, 95/2.5/2.5, v/v/v, 100 mL) was added to the flask containing the side chain protected peptide resin (intermediate 55, 5.0 g) at room temperature and stirred for 2h.
2) The filtrate was collected.
3) The peptide was precipitated with cold isopropyl ether (500 mL) and centrifuged (3 min at 3000 rpm).
4) Isopropyl ether washed two additional times, and the crude peptide was dried under vacuum for 2 h to afford Compound 182 (3.10 g, crude) as a white solid.
5) The crude was purified by prep-HPLC (A: 0.075% TFA/H₂O, B: MeCN), followed by lyophilization to afford 0.50 mmol resin afforded Compound 105 (40.5 mg, 95.0% purity, 1.18% yield) as a white solid. LCMS: RT=1.16 min, MS cal.: 6851.79, [M+5H]⁵⁺=1371.45, [M+6H]⁶⁺=1143.05, [M+7H]⁷⁺=979.59, [M+8H]⁸⁺=857.75, [M+9H]⁹⁺=762.22.

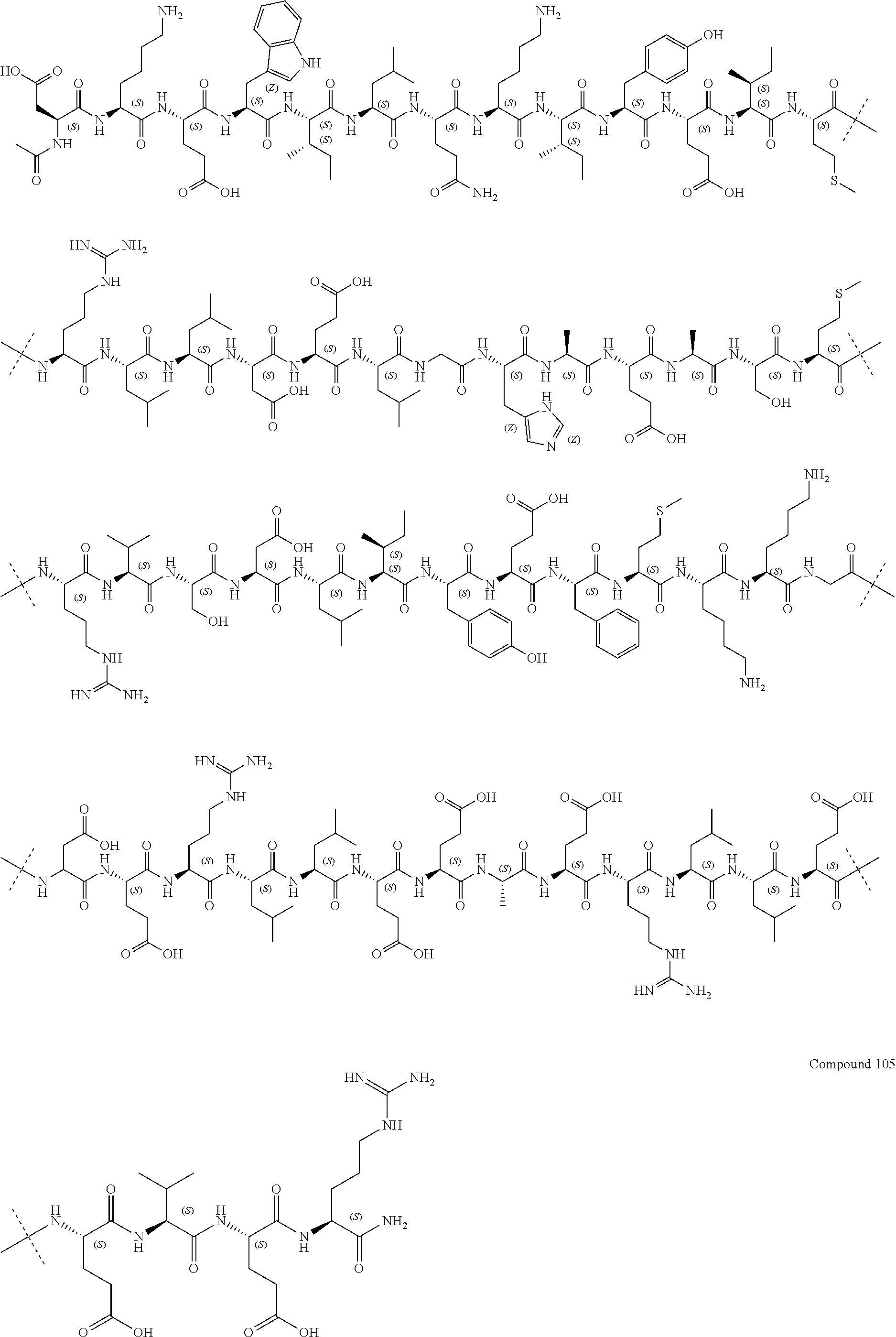

(SEQ ID NO: 86)
Ace-DKEWILQKIYEIMRLLDELGHAEASMRVSDLIYEFMKKGDERLLEE
AERLLEEVER-NH2

Compounds 195, 196, 197, 198, 199, 200, 201, 202 were synthesized using the same procedure of peptide synthesis standard Fmoc chemistry, peptide cleavage and purification as Compound 105.

0.50 mmol resin afforded Compound 195 (69.4 mg, 86.7% purity, 1.78% yield) as a white solid. LCMS: RT=1.10 min, MS cal.: 7777.84, [M+6H]⁶⁺=1297.26, [M+7H]⁷⁺=1112.30, [M+8H]⁸⁺973.41, [M+10H]¹⁰⁺=778.84.

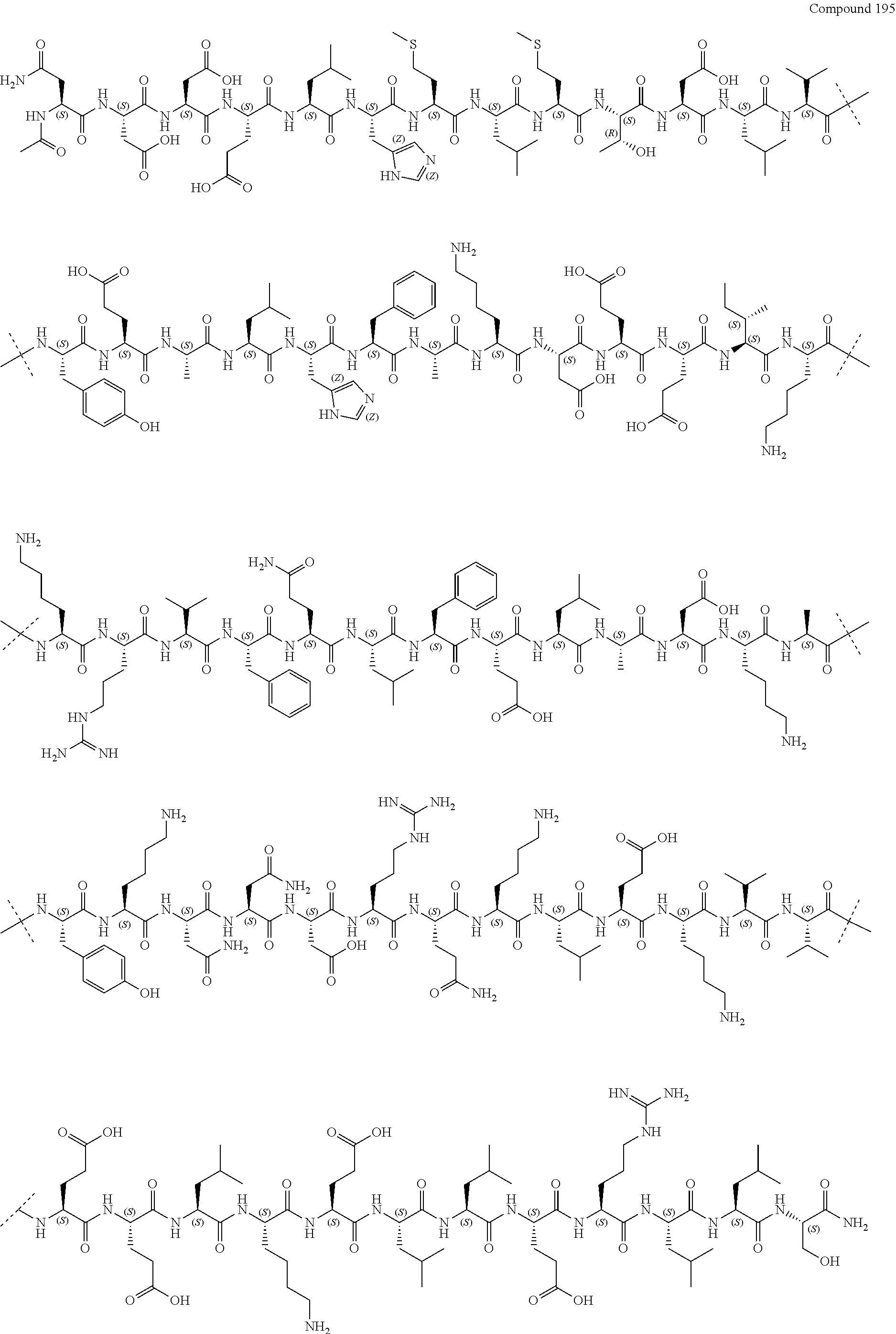

(SEQ ID NO: 87)
Ace-
NDDELHMLMTDLVYEALHFAKDEEIKKRVFQLFELADKAYKNNDRQKLEK
VVEELKELLERLLS-NH2

0.50 mmol resin afforded Compound 196 (28.0 mg, 87.8% purity, 0.82% yield) as a white solid. LCMS: RT=1.25 min, MS cal.: 6815.71, [M+5H]⁵⁺=1363.77, [M+6H]⁶⁺=1136.55, [M+7H]7=974.65, [M+8H]⁸⁺=852.75, [M+9H]⁹⁺=758.10.

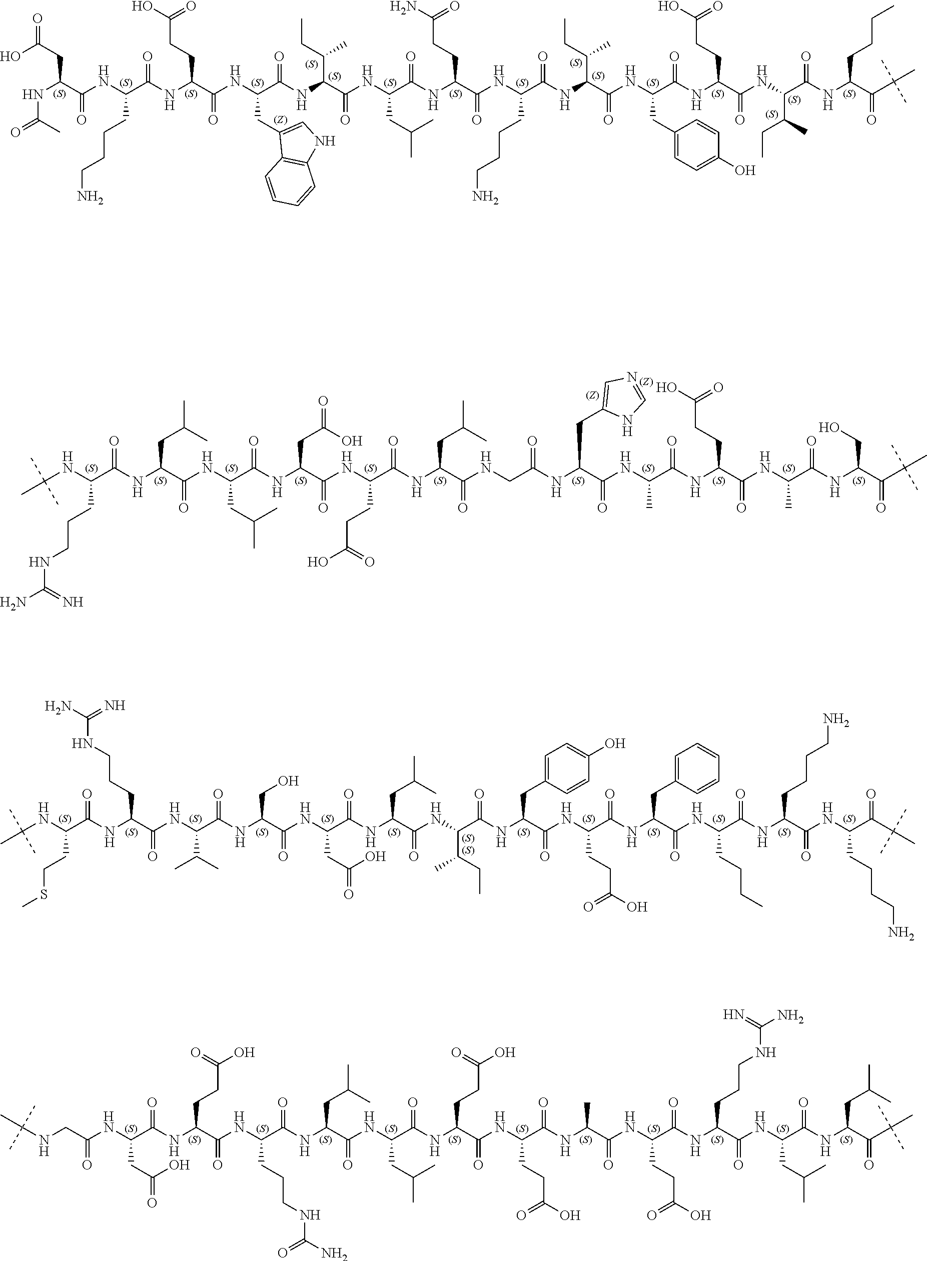

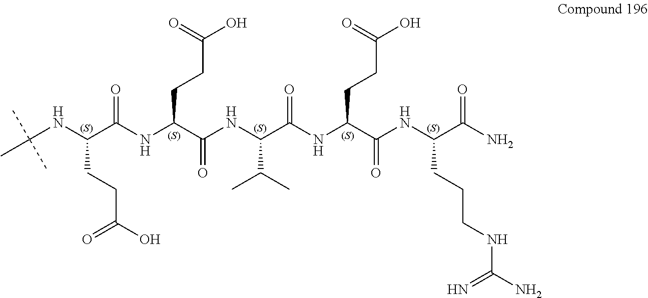

0.50 mmol resin afforded Compound 197 (Batch1: 41.7 mg, 96.1% purity; batch2: 47.4 mg, 93.3% purity; 3.76% yield) as a white solid. LCMS: RT=2.18 min, MS cal.: 4741.51, [M+3H]³⁺=1581.00, [M+4H]⁴⁺+=1186.10, [M+5H]⁵⁺=949.30, [M+6H]⁶⁺=791.10.

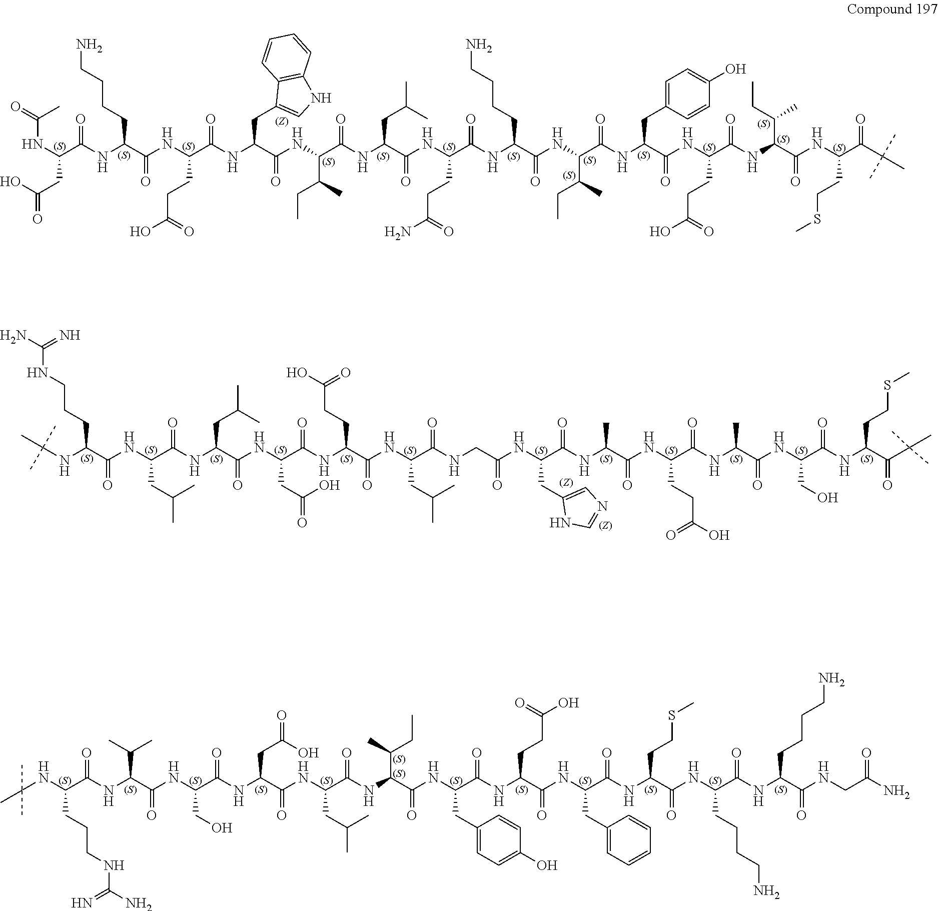

0.20 mmol resin afforded Compound 198 (4.8 mg, 86.7% purity, 0.30% yield) as a white solid. TOF: RT=0.517 min, MS cal.: 6931.88, [M+4H]⁴⁺=1734.14, [M+5H]⁵⁺=1387.11, [M+6H]⁶⁺=1156.09, [M+7H]⁷⁺=991.08.

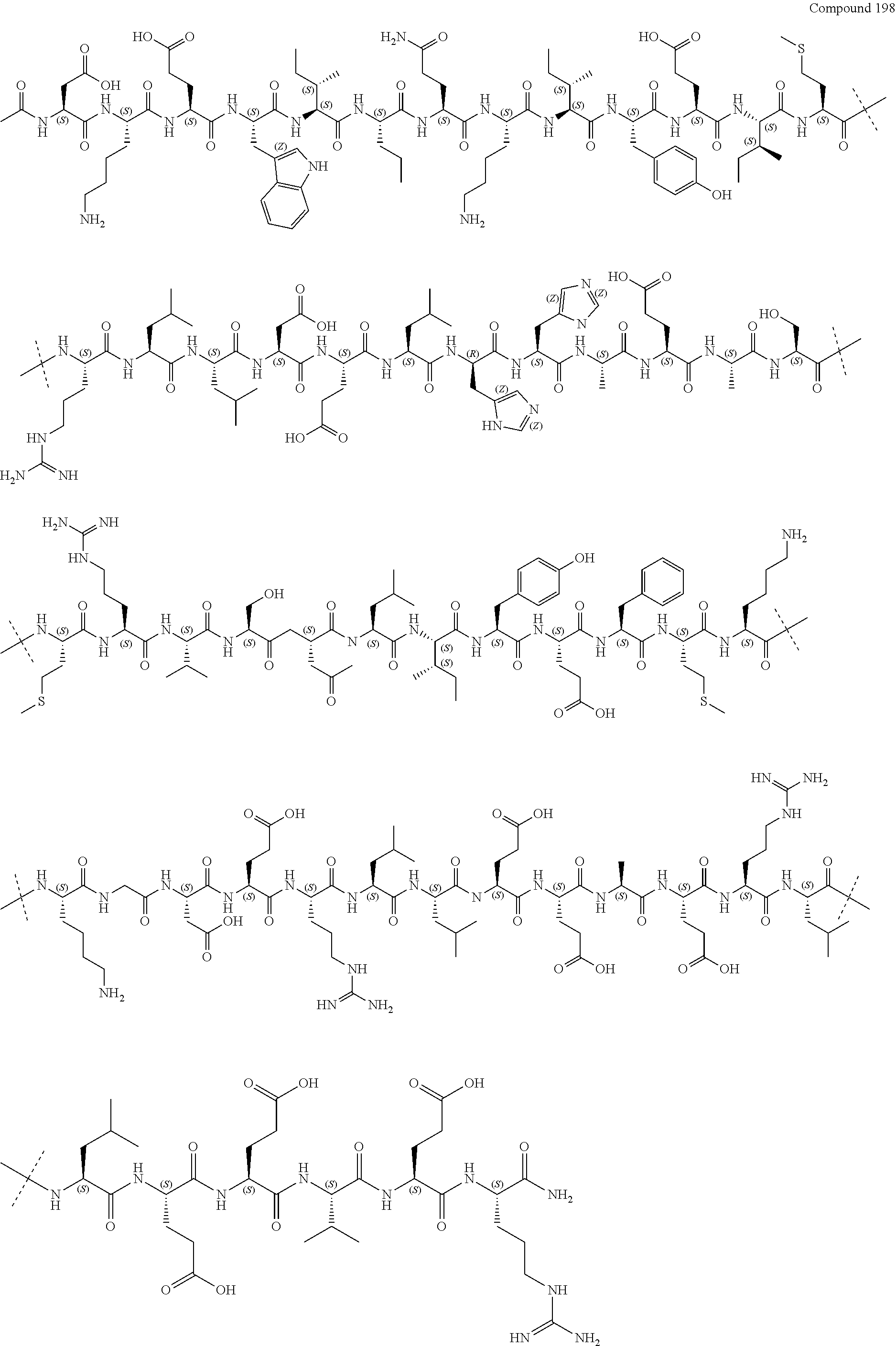

(SEQ ID NO: 88)
Ace-DKEWILQKIYEIMRLLDEL[DHis]HAPASMRVSDLIYEFMKKGDE
RLLEEAERLLEEVER-NH2

0.20 mmol resin afforded Compound 199 (16.3 mg, 92.0% purity, 1.09% yield) as a white solid. LCMS: RT=1.26 min, MS cal.: 6819.79, [M+5H]⁵⁺=1364.85, [M+6H]⁶⁺=1137.16, [M+7H]⁷⁺=975.40, [M+8H]⁸⁺=853.48.

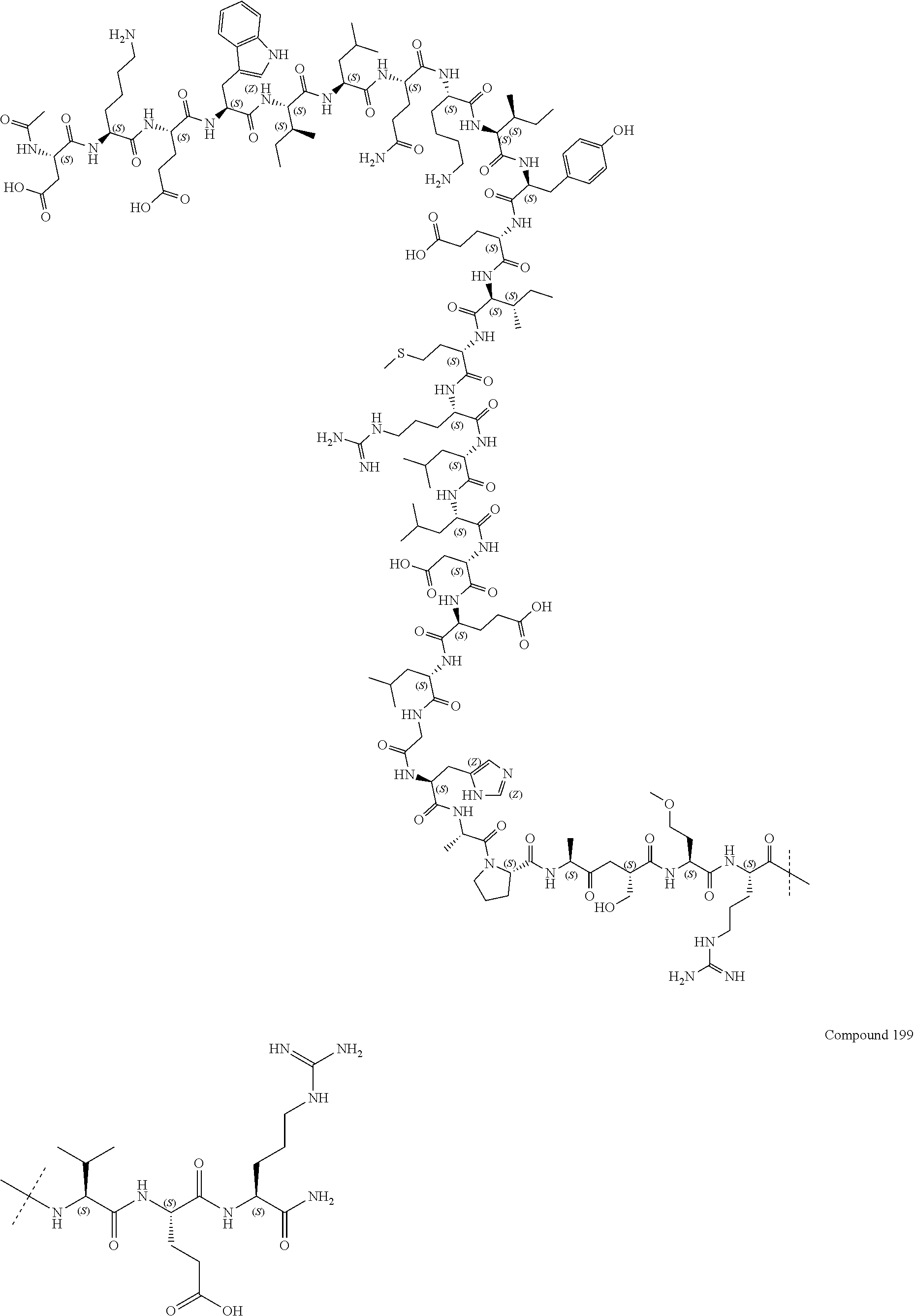

0.20 mmol resin afforded Compound 200 (32.4 mg, 92.3% purity, 2.12% yield) as a white solid. LCMS: RT=0.517 min, MS cal.: 7037.00, [M+5H]⁵⁺=1407.73, [M+6H]⁶⁺=1173.76, [M+7H]⁷+=880.66, [M+8H]⁸⁺=782.74.

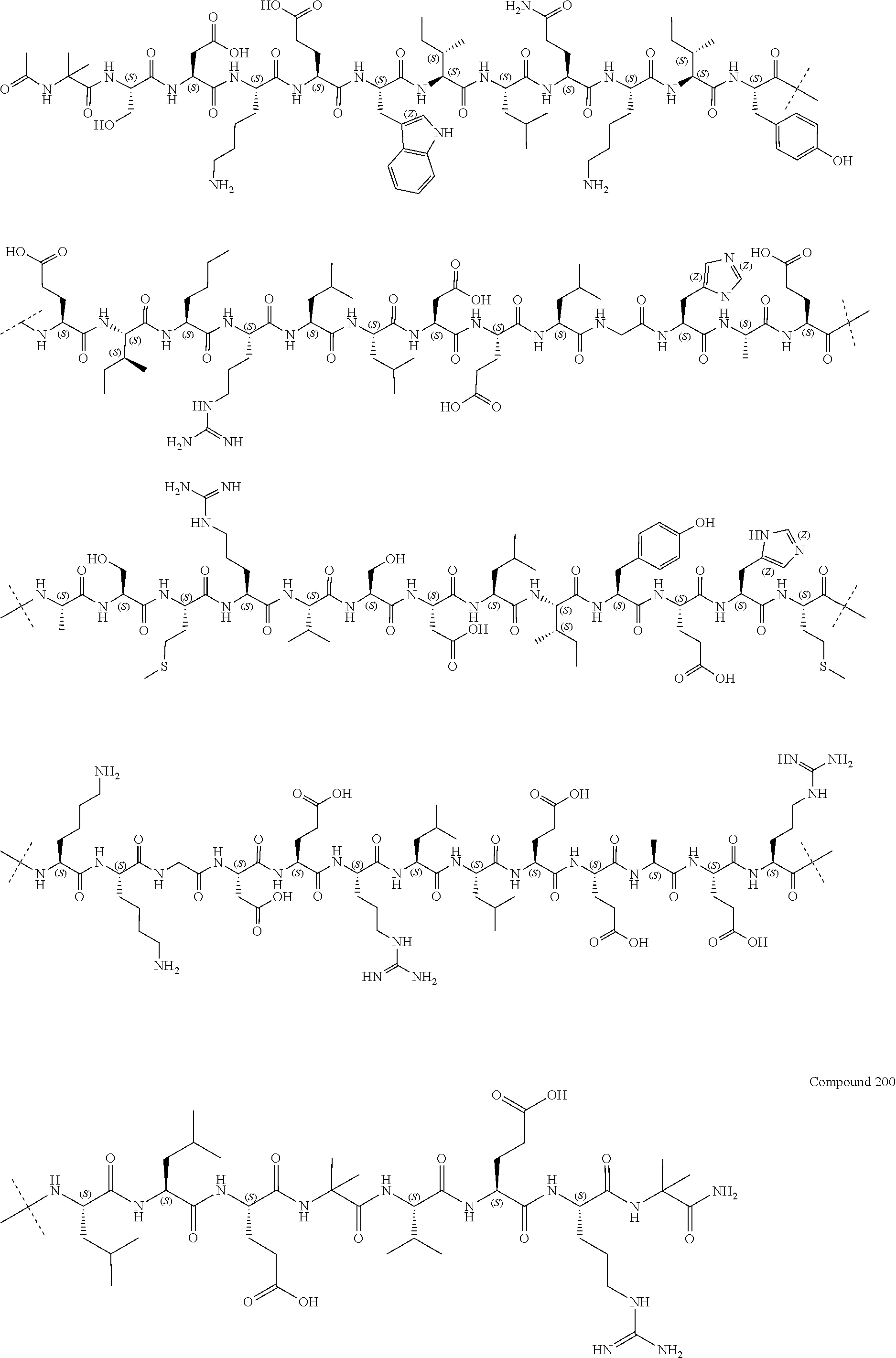

0.20 mmol resin afforded Compound 201 (21.4 mg, 90.7% purity, 1.40% yield) as a white solid. LCMS: RT=1.06 min, MS cal.: 6931.88, [M+4H]⁴⁺+=1733.84, [M+5H]⁵⁺=1387.28, [M+6H]⁶⁺=1156.01, [M+7H]⁷⁺=991.10, [M+8H]⁸⁺=867.19, [M+9H]⁹⁺=770.99.

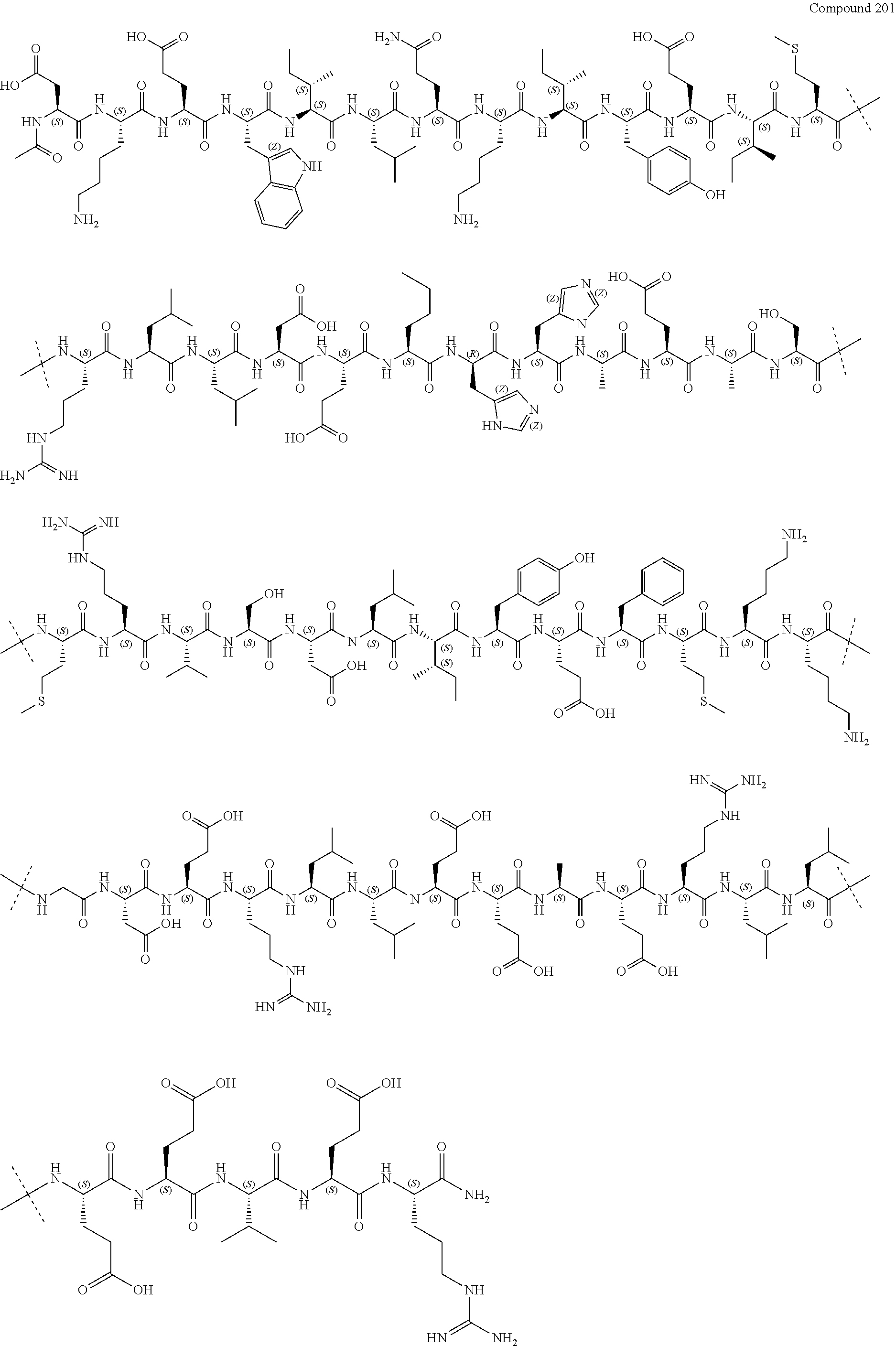

(SEQ ID NO: 89)
Ace-DKEWILQKIYEIMRLLDE[Nle][DHis]HAEASMRVSDLIYEFM
KKGDERLLEEAERLLEEVER-NH2

0.20 mmol resin afforded Compound 202 (76.6 mg, 95.9% purity, 7.66% yield) as a white solid. LCMS: RT=1.11 min, MS cal.: 4796.59, [M+3H]³⁺=1599.30, [M+4H]⁴⁺=1199.76, [M+5H]⁵⁺=960.00, [M+6H]⁶⁺=799.82.

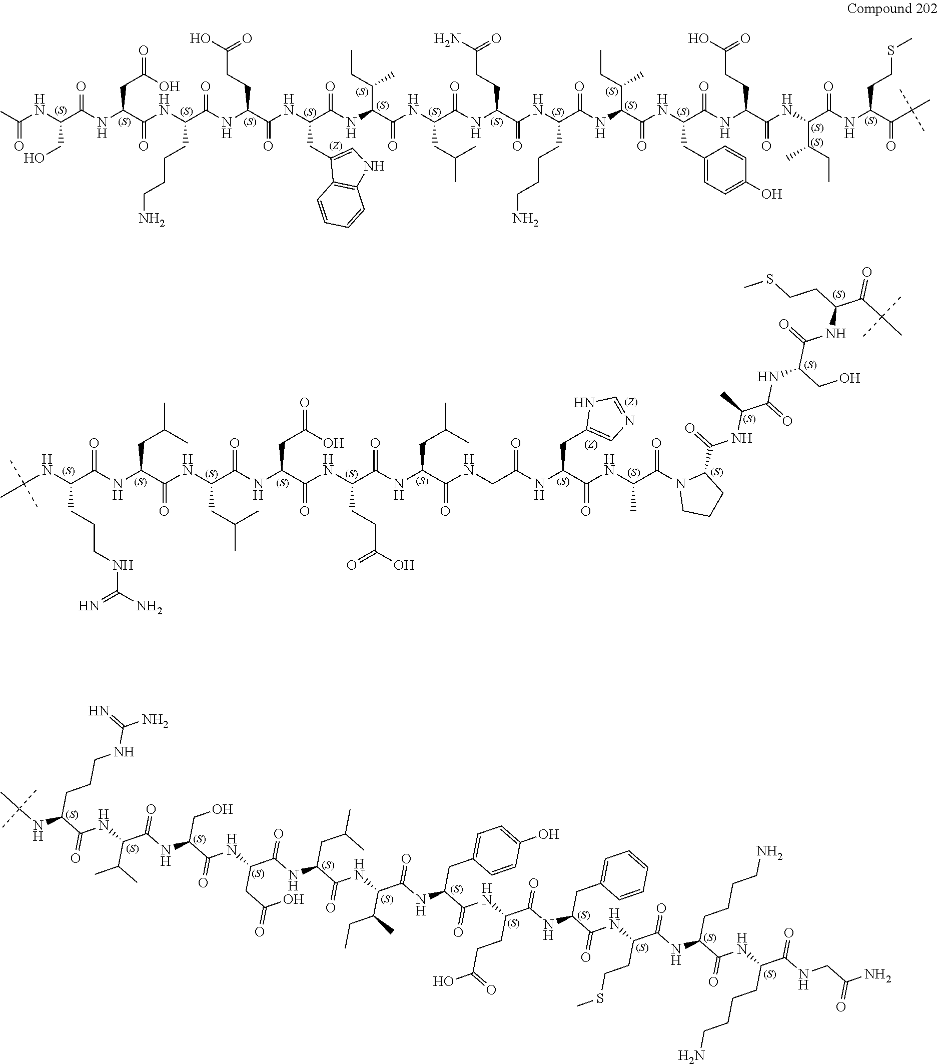

### Example 20. Procedure for Preparation of Compound 203 (12 Families, Linear Peptide, CTC Resin)

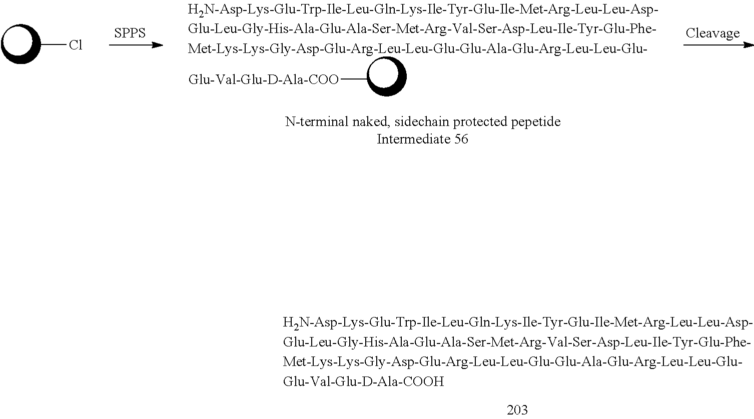

Peptide was synthesized using standard Fmoc chemistry (CTC resin).
1) Resin preparation: To the vessel containing CTC Resin (1.0 mmol, 1.0 g, 1.00 mmol/g) and Fmoc-D-Ala-OH (311.5 mg, 1.0 mmol, 1.00 equiv.) in DCM (10 mL) was added DIEA (4.00 equiv.) dropwise and mixed for 2 h with N₂ bubbling at 20° C. Then MeOH (1.0 mL) was added and bubbled with N₂ for another 30 min. The resin was washed with DMF (20 mL)*5, followed by adding 20% piperidine in DMF (20 mL) and bubbled with N₂ for 30 min at 20° C. for Fmoc deprotection. The mixture was filtered and the resin was washed with DMF (20 mL)*5 before proceeding to next step.
2) Coupling: A solution of Fmoc-Glu(OtBu)-OH (1.28 g, 3.00 equiv.), HBTU (1.08 g, 2.85 equiv.) in DMF (10 mL) was added to the resin with N₂ bubbling. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (20 mL)*5.
3) Deprotection: 20% piperidine in DMF (20 mL) was added to the resin and the mixture was bubbled with N₂ for 30 min at 20° C. The deprotection reaction was monitored by ninhydrin test, if it showed blue or other brownish red, the reaction was completed. The resin was then washed with DMF (20 mL)*5.
4) Step 2 and 3 were repeated for amino acids: (2-13 in Table 16).
5) Coupling: A solution of Fmoc-Leu-OH (1.06 g, 3.00 equiv.), HATU (1.08 g, 2.85 equiv.) in DMF (10 mL) was added to the resin with N₂ bubbling for 30 min at 20° C. Then DIEA (6.00 equiv.) was added to the mixture dropwise and bubbled with N₂ for 30 min at 20° C. The coupling reaction was monitored by ninhydrin test, if it showed colorless, the coupling was completed. The resin was then washed with DMF (30 mL)*5.
6) Repeat step 3 for Fmoc deprotection.
7) Step 5 and 6 were repeated for amino acids (15-56 in Table 16).
8) After the last position completed, the resin was then washed with DMF (200 mL)*5, MeOH (200 mL)*5, and then dried under vacuum to afford peptide resin intermediate 56 (SEQ ID NO:90) (CTC resin, 1 mmol, 12.00 g).

TABLE 16
The list of amino acids and the corresponding reagents used on SPPS.

| # | Materials | Coupling reagents |
|---|---|---|
| 1 | Fmoc-D-Ala-OH (1.00 equiv.) | DIEA (4.00 equiv.) |
| 2 | Fmoc-Glu(OtBu)-OH (3.00 equiv.) | HBTU (2.85 equiv.) and DIEA (6.00 equiv.) |
| 3 | Fmoc-Val-OH (3.00 equiv.) | HBTU (2.85 equiv.) and DIEA (6.00 equiv.) |
| 4 | Fmoc-Glu(OtBu)-OH (3.00 equiv.) | HBTU (2.85 equiv.) and DIEA (6.00 equiv.) |
| 5 | Fmoc-Glu(OtBu)-OH (3.00 equiv.) | HBTU (2.85 equiv.) and DIEA (6.00 equiv.) |
| 6 | Fmoc-Leu-OH (3.00 equiv.) | HBTU (2.85 equiv.) and DIEA (6.00 equiv.) |
| 7 | Fmoc-Leu-OH (3.00 equiv.) | HBTU (2.85 equiv.) and DIEA (6.00 equiv.) |
| 8 | Fmoc-Arg(Pbf)-OH (3.00 equiv.) | HBTU (2.85 equiv.) and DIEA (6.00 equiv.) |
| 9 | Fmoc-Glu(OtBu)-OH (3.00 equiv.) | HBTU (2.85 equiv.) and DIEA (6.00 equiv.) |
| 10 | Fmoc-Ala-OH (3.00 equiv.) | HBTU (2.85 equiv.) and DIEA (6.00 equiv.) |
| 11 | Fmoc-Glu(OtBu)-OH (3.00 equiv.) | HBTU (2.85 equiv.) and DIEA (6.00 equiv.) |
| 12 | Fmoc-Glu(OtBu)-OH (3.00 equiv.) | HBTU (2.85 equiv.) and DIEA (6.00 equiv.) |
| 13 | Fmoc-Leu-OH (3.00 equiv.) | HBTU (2.85 equiv.) and DIEA (6.00 equiv.) |
| 14 | Fmoc-Leu-OH (3.00 equiv.) | HATU (2.85 equiv.) and DIEA (6.00 equiv.) |
| 15 | Fmoc-Arg(Pbf)-OH (3.00 equiv.) | HATU (2.85 equiv.) and DIEA (6.00 equiv.) |
| 16 | Fmoc-Glu(OtBu)-OH (3.00 equiv.) | HATU (2.85 equiv.) and DIEA (6.00 equiv.) |
| 17 | Fmoc-Asp(OtBu)-OH (3.00 equiv.) | HATU (2.85 equiv.) and DIEA (6.00 equiv.) |
| 18 | Fmoc-Gly-OH (3.00 equiv.) | HATU (2.85 equiv.) and DIEA (6.00 equiv.) |
| 19 | Fmoc-Lys(Boc)-OH (3.00 equiv.) | HATU (2.85 equiv.) and DIEA (6.00 equiv.) |
| 20 | Fmoc-Lys(Boc)-OH (3.00 equiv.) | HATU (2.85 equiv.) and DIEA (6.00 equiv.) |
| 21 | Fmoc-Met-OH (3.00 equiv.) | HATU (2.85 equiv.) and DIEA (6.00 equiv.) |
| 22 | Fmoc-Phe-OH (3.00 equiv.) | HATU (2.85 equiv.) and DIEA (6.00 equiv.) |
| 23 | Fmoc-Glu(OtBu)-OH (3.00 equiv.) | HATU (2.85 equiv.) and DIEA (6.00 equiv.) |
| 24 | Fmoc-Tyr(tBu)-OH (3.00 equiv.) | HATU (2.85 equiv.) and DIEA (6.00 equiv.) |
| 25 | Fmoc-Ile-OH (3.00 equiv.) | HATU (2.85 equiv.) and DIEA (6.00 equiv.) |
| 26 | Fmoc-Leu-OH (3.00 equiv.) | HATU (2.85 equiv.) and DIEA (6.00 equiv.) |
| 27 | Fmoc-Asp(OtBu)-OH (3.00 equiv.) | HATU (2.85 equiv.) and DIEA (6.00 equiv.) |
| 28 | Fmoc-Ser(tBu)-OH (3.00 equiv.) | HATU (2.85 equiv.) and DIEA (6.00 equiv.) |
| 29 | Fmoc-Val-OH (3.00 equiv.) | HATU (2.85 equiv.) and DIEA (6.00 equiv.) |
| 30 | Fmoc-Arg(Pbf)-OH (3.00 equiv.) | HATU (2.85 equiv.) and DIEA (6.00 equiv.) |
| 31 | Fmoc-Met-OH (3.00 equiv.) | HATU (2.85 equiv.) and DIEA (6.00 equiv.) |
| 32 | Fmoc-Ser(tBu)-OH (3.00 equiv.) | HATU (2.85 equiv.) and DIEA (6.00 equiv.) |
| 33 | Fmoc-Ala-OH (3.00 equiv.) | HATU (2.85 equiv.) and DIEA (6.00 equiv.) |
| 34 | Fmoc-Glu(OtBu)-OH (3.00 equiv.) | HATU (2.85 equiv.) and DIEA (6.00 equiv.) |
| 35 | Fmoc-Ala-OH (3.00 equiv.) | HATU (2.85 equiv.) and DIEA (6.00 equiv.) |
| 36 | Fmoc-His(Trt)-OH (3.00 equiv.) | HATU (2.85 equiv.) and DIEA (6.00 equiv.) |
| 37 | Fmoc-Gly-OH (3.00 equiv.) | HATU (2.85 equiv.) and DIEA (6.00 equiv.) |
| 38 | Fmoc-Leu-OH (3.00 equiv.) | HATU (2.85 equiv.) and DIEA (6.00 equiv.) |
| 39 | Fmoc-Glu(OtBu)-OH (3.00 equiv.) | HATU (2.85 equiv.) and DIEA (6.00 equiv.) |
| 40 | Fmoc-Asp(OtBu)-OH (3.00 equiv.) | HATU (2.85 equiv.) and DIEA (6.00 equiv.) |
| 41 | Fmoc-Leu-OH (3.00 equiv.) | HATU (2.85 equiv.) and DIEA (6.00 equiv.) |
| 42 | Fmoc-Leu-OH (3.00 equiv.) | HATU (2.85 equiv.) and DIEA (6.00 equiv.) |
| 43 | Fmoc-Arg(Pbf)-OH (3.00 equiv.) | HATU (2.85 equiv.) and DIEA (6.00 equiv.) |
| 44 | Fmoc-Met-OH (3.00 equiv.) | HATU (2.85 equiv.) and DIEA (6.00 equiv.) |
| 45 | Fmoc-Ile-OH (3.00 equiv.) | HATU (2.85 equiv.) and DIEA (6.00 equiv.) |
| 46 | Fmoc-Glu(OtBu)-OH (3.00 equiv.) | HATU (2.85 equiv.) and DIEA (6.00 equiv.) |
| 47 | Fmoc-Tyr(tBu)-OH (3.00 equiv.) | HATU (2.85 equiv.) and DIEA (6.00 equiv.) |
| 48 | Fmoc-Ile-OH (3.00 equiv.) | HATU (2.85 equiv.) and DIEA (6.00 equiv.) |
| 49 | Fmoc-Lys(Boc)-OH (3.00 equiv.) | HATU (2.85 equiv.) and DIEA (6.00 equiv.) |
| 50 | Fmoc-Gln(Trt)-OH (3.00 equiv.) | HATU (2.85 equiv.) and DIEA (6.00 equiv.) |
| 51 | Fmoc-Leu-OH (3.00 equiv.) | HATU (2.85 equiv.) and DIEA (6.00 equiv.) |
| 52 | Fmoc-Ile-OH (3.00 equiv.) | HATU (2.85 equiv.) and DIEA (6.00 equiv.) |
| 53 | Fmoc-Trp-OH (3.00 equiv.) | HATU (2.85 equiv.) and DIEA (6.00 equiv.) |
| 54 | Fmoc-Glu(OtBu)-OH (3.00 equiv.) | HATU (2.85 equiv.) and DIEA (6.00 equiv.) |
| 55 | Fmoc-Lys(Boc)-OH (3.00 equiv.) | HATU (2.85 equiv.) and DIEA (6.00 equiv.) |
| 56 | Fmoc-Asp(OtBu)-OH (3.00 equiv.) | HATU (2.85 equiv.) and DIEA (6.00 equiv.) |

### Peptide Cleavage and Purification:

1) Cleavage buffer (TFA/TIS/H₂O/3-mercaptopropanoic acid, 92.5/2.5/2.5/2.5, v/v/v, 20 mL) was added to the flask containing the side chain protected peptide resin (intermediate 6, 0.1 mmol, 1.20 g) at room temperature and stirred for 2 h.
2) After filtration, the filtrate was collected.
3) The peptide was precipitated with cold isopropyl ether (100 mL) and centrifuged (3 min at 3000 rpm).
4) Isopropyl ether washed two additional times, and the crude peptide was dried under vacuum for 2 h to afford crude peptide (0.80 g, crude) as a white solid.
5) The crude was purified by prep-HPLC (A: 0.075% TFA/H₂O, B: MeCN), followed by lyophilization to afford Compound 203 (SEQ ID NO:90) (17.1 mg, 78.3% purity, 1.99% yield) as a white solid. LCMS: RT=1.18 min, MS cal.: 6725.77, [M+5H]⁵⁺=1346.16, [M+6H]⁶⁺=1121.60, [M+7H]⁷⁺=961.72, [M+8H]⁸⁺=841.34.

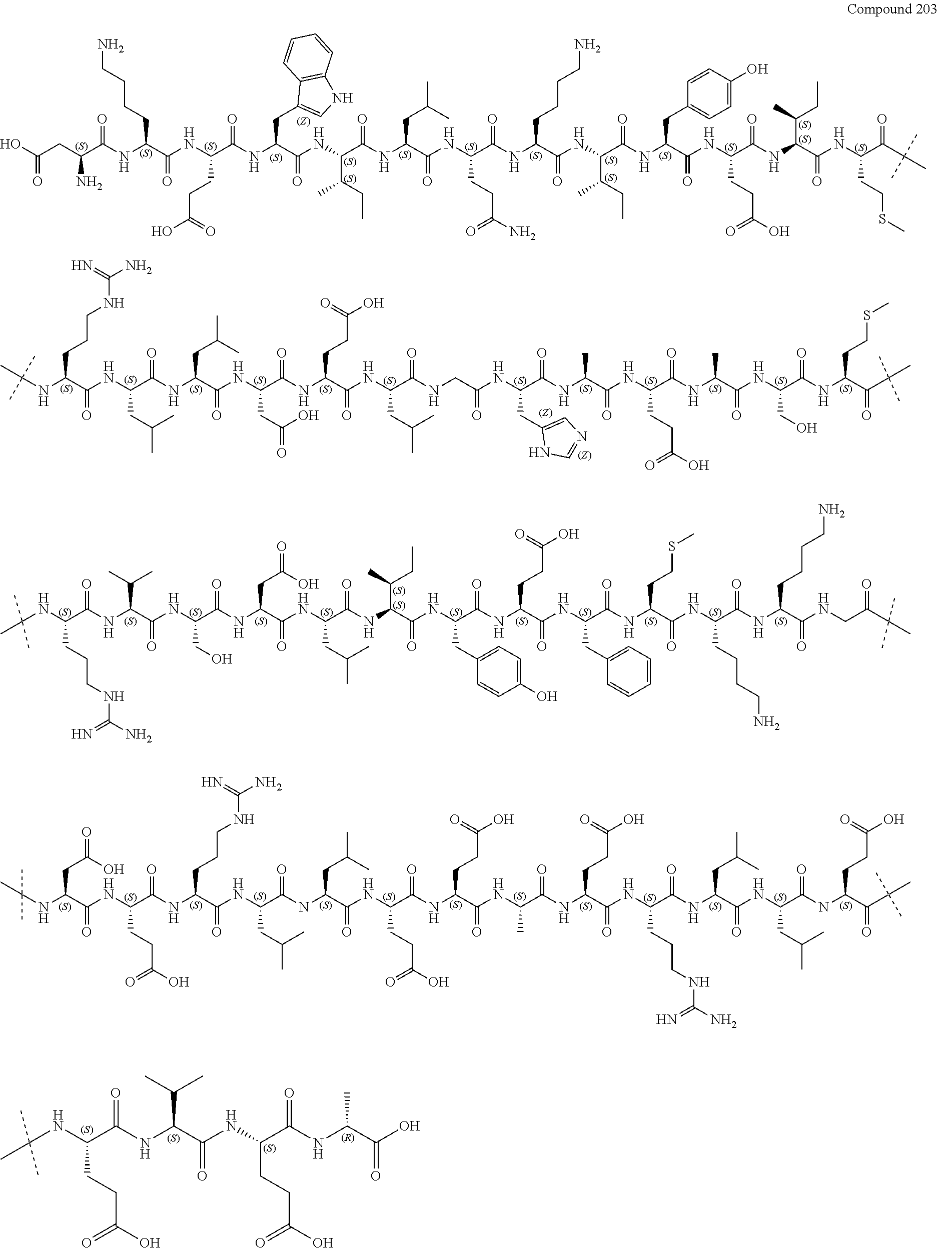

(SEQ ID NO: 91)
DKEWILQKIYEIMRLLDELGHAPASMRVSDLIYEFMKKGDERLLEEAERL
LEEVE[DAla][DAla]

Compound 204), 205, 206, 207, 208, 102, 209, 210, 211, 212, 213, 214, 215, 256, 216, 217, 218, 219, 220, 221, and 222 were synthesized using the same method as Compound 203.

0.10 mmol resin afforded Compound 204 obtaining (23.8 mg, 84.0% purity, 2.95% yield) as a white solid. LCMS: RT=1.20 min, MS cal.: 6766.87, [M+4H]⁴⁺+=1692.46, [M+5H]⁵⁺=1354.03, [M+6H]⁶⁺=1128.65, [M+7H]⁷⁺=967.47, [M+8H]⁸⁺=846.60.

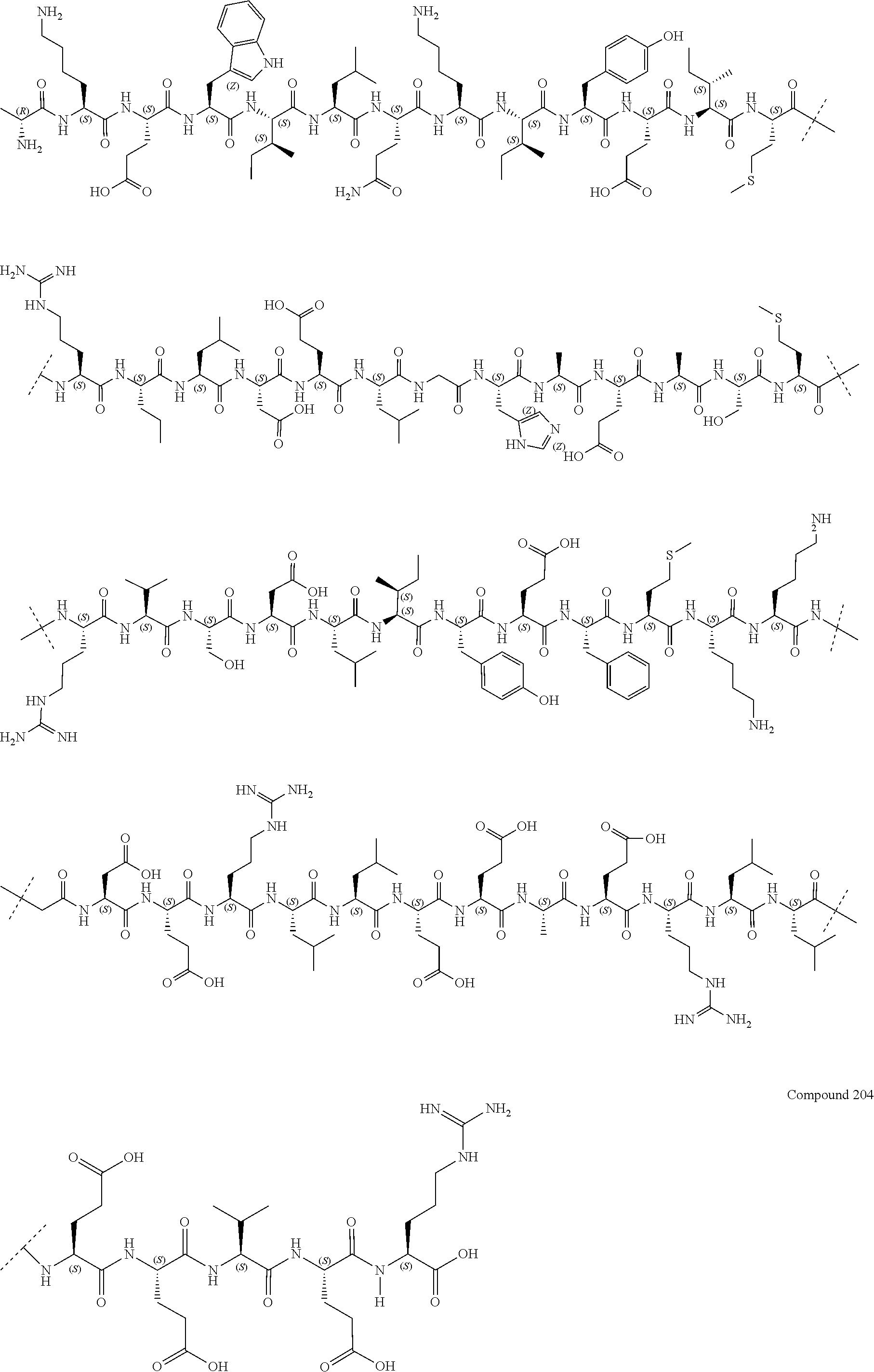

(SEQ ID NO: 92)
[DAla]KEWILQKIYEIMRLLDELGHAPASMRVSDLIYEFMKKGDERLLE
EAERLLEEVER

0.50 mmol resin afforded Compound 205 (20.5 mg, 90.1% purity, 0.60% yield) as a white solid. LCMS: RT=1.13 min, MS cal.: 6810.74, [M+5H]⁵⁺=1363.06, [M+6H]⁶⁺=1136.06, [M+7H]⁷⁺=974.22, [M+8H]⁸⁺=852.62, [M+9H]⁹⁺=757.77.

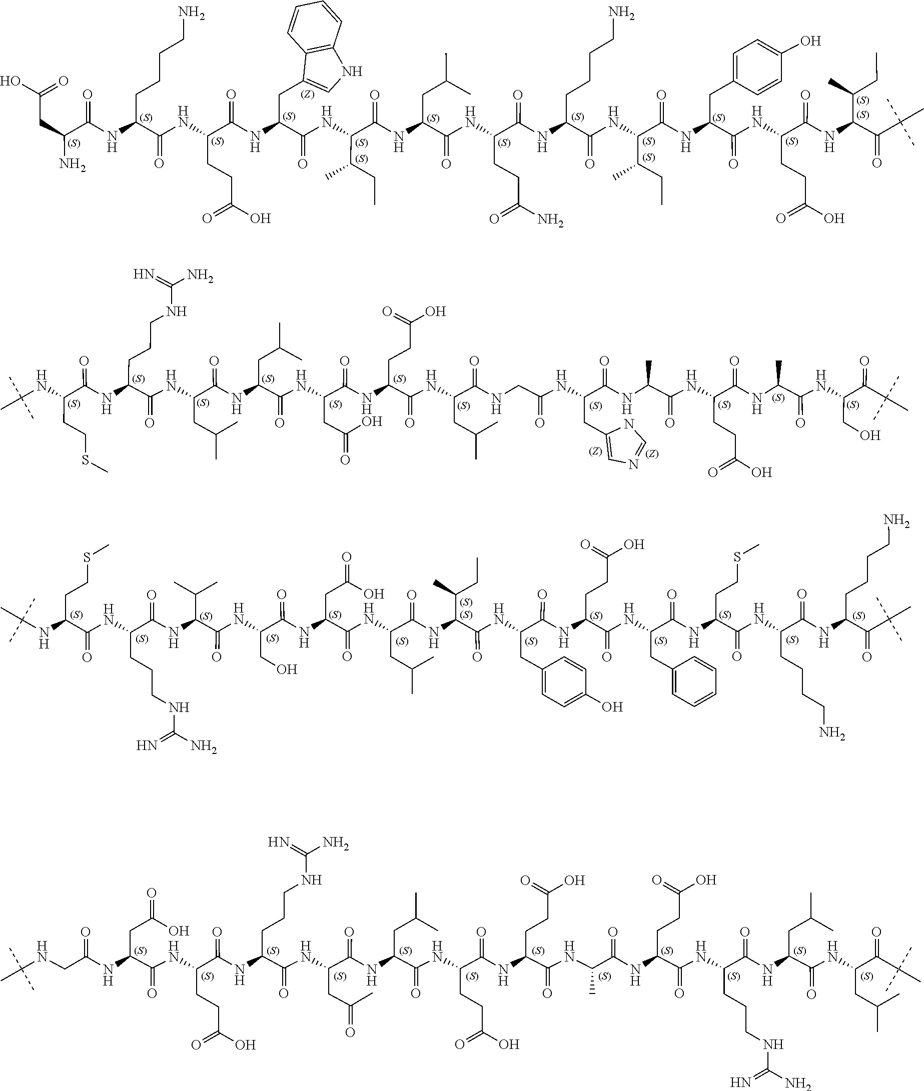

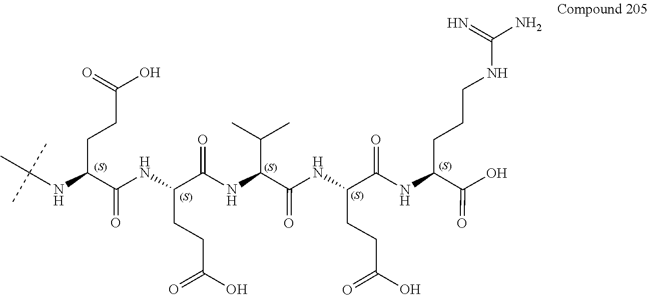

0.50 mmol resin afforded Compound 206 (315.4 mg, 91.4% purity, 8.03% yield) as a white solid. LCMS: RT=2.32 min, MS cal.: 7858.96, [M+4H]⁴⁺=1965.20, [M+5H]*+=1572.50, [M+6H]⁶⁺=1310.60, [M+7H]⁷⁺=1123.50, [M+8H]⁸⁺=983.10, [M+9H]⁹⁺=874.00, [M+10H]^{10+=786.80}, [M+11H]¹¹⁺+=715.50, [M+12H]¹²⁺=655.90.

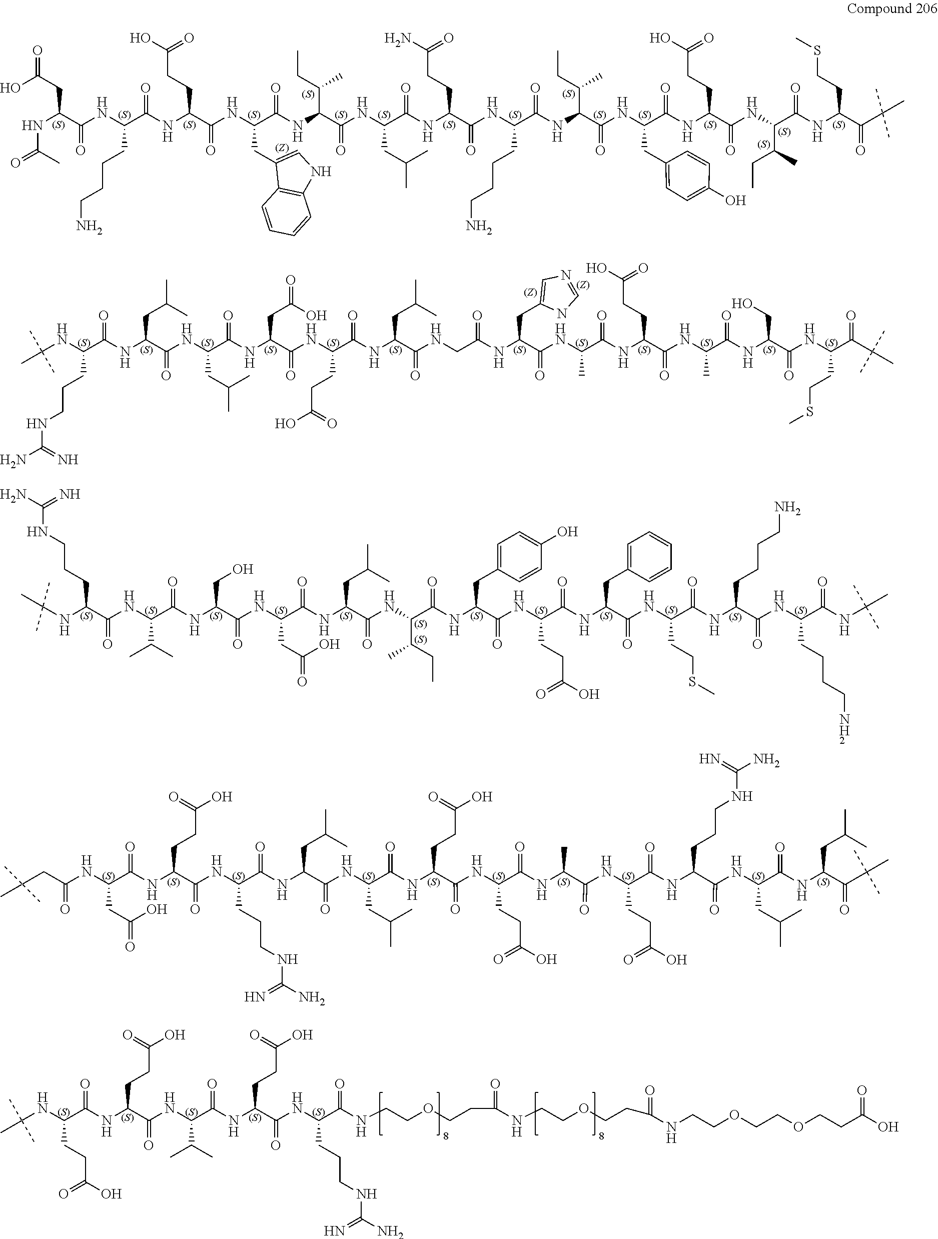

(SEQ ID NO: 93)
Ac-DKEWILQKIYEIMRLLDELGHAEASMRVSDLIYEFMKKGDERLLEEA
ERLLEEVER-PEG8-PEG8-PEG2-COOH

0.50 mmol resin afforded Compound 207 (25.1 mg, 91.1% purity, 0.69% yield) as a white solid. LCMS: RT=1.09 min, MS cal.: 7234.24, [M+4H]⁴⁺+=1809.25, [M+5H]⁵⁺=1447.22, [M+6H]⁶⁺=1206.07, [M+8H]⁸⁺=904.92, [M+9H]⁹⁺=804.48, [M+10H]¹⁰⁺=724.20.

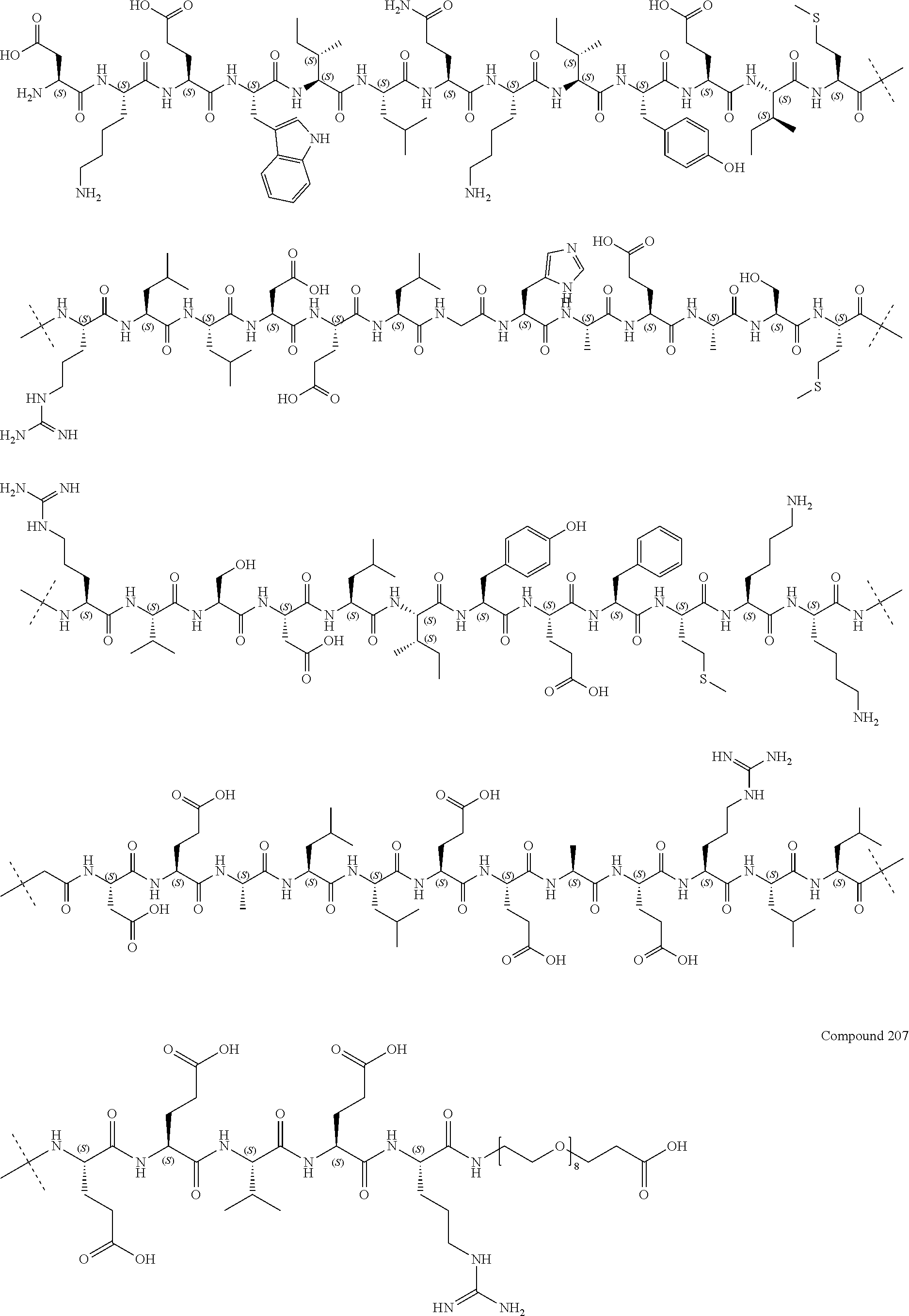

0.50 mmol resin afforded Compound 208 (15.1 mg, 87.1% purity, 0.39% yield) as a white solid. LCMS: RT=1.07 min, MS cal.: 7707.82, [M+4H]⁴⁺+=1927.92, [M+5H]⁵⁺=1542.25, [M+6H]⁶⁺=1285.39, [M+7H]⁷⁺=1102.00, [M+8H]⁸⁺=964.21, [M+9H]⁹⁺=857.26, [M+10H]¹⁰⁺=771.40.

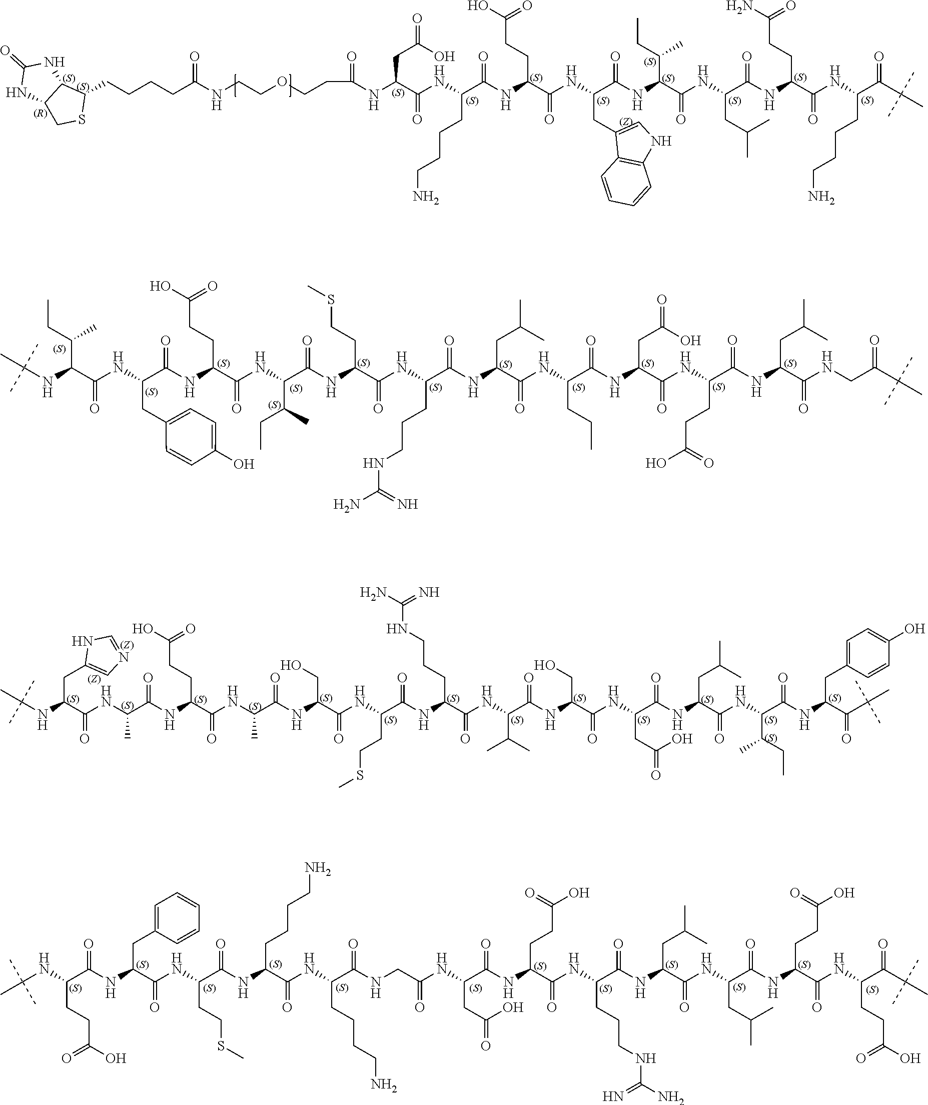

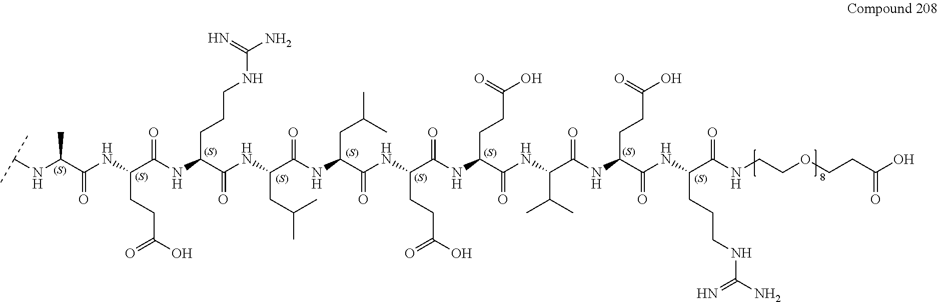

0.20 mmol resin afforded Compound 102 (35.4 mg, 91.5% purity, 3.44% yield) as a white solid. LCMS: RT=1.08 min, MS cal.:4700.46, [M+3H]³⁺=1568.17, [M+4H]⁴⁺=1175.85, [M+6H]⁶⁺=783.98.

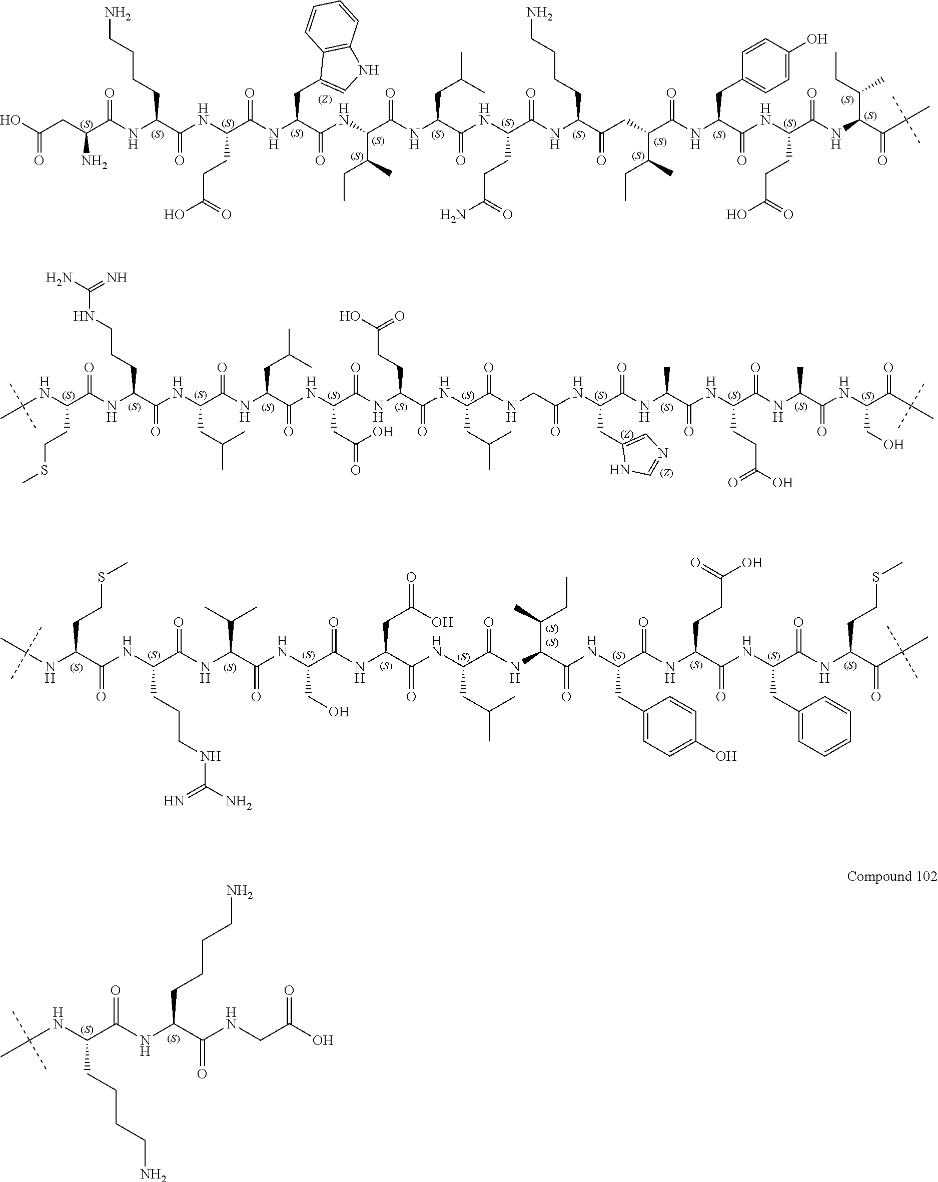

0.20 mmol resin afforded Compound 209 (25.9 mg, 98.1% purity, 2.45% yield) as a white solid. LCMS: RT=1.08 min, MS cal.:5174.04, [M+3H]³⁺=1725.42, [M+4H]⁴⁺=1294.54, [M+5H]⁵⁺=1035.75, [M+7H]⁷⁺=704.30.

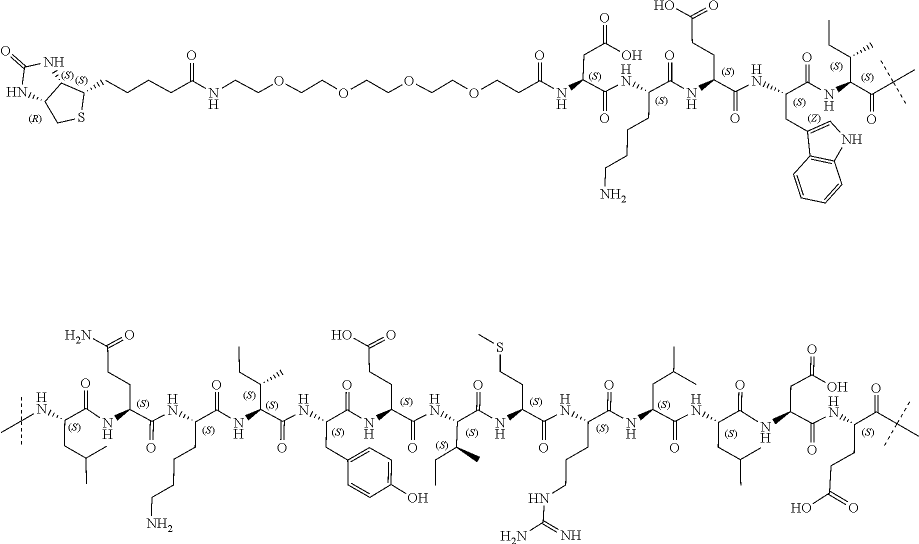

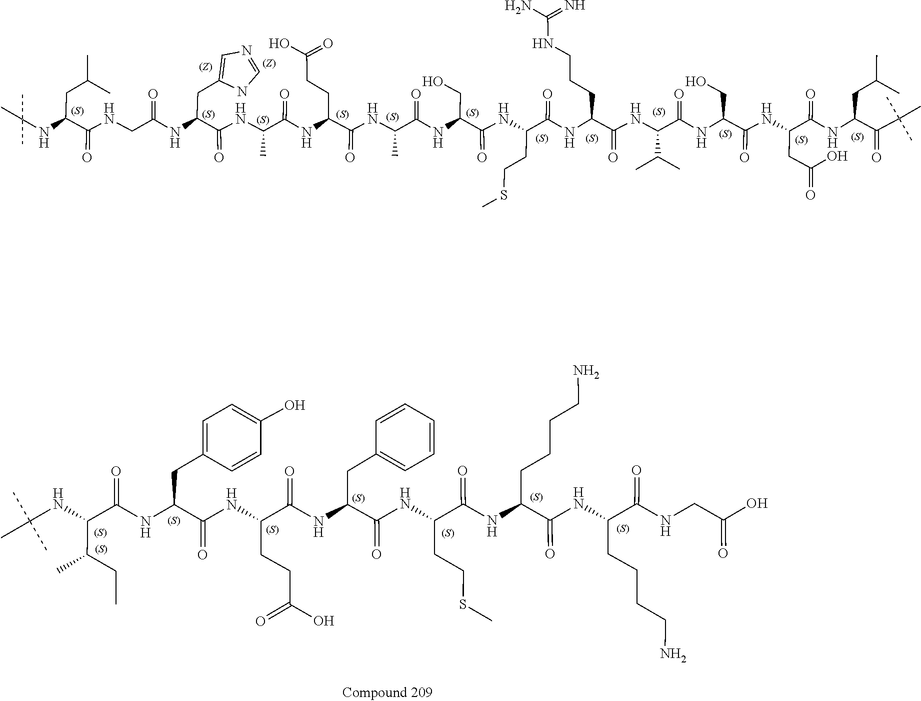

0.20 mmol resin afforded Compound 210 (53.1 mg, 97.7% purity, 5.38% yield) as a white solid. TOF: RT=0.51 min, MS cal.:4185.69, [M+3H]³⁺=1396.03, [M+4H]⁴⁺=1047.02, [M+5H]⁵⁺=838.02.

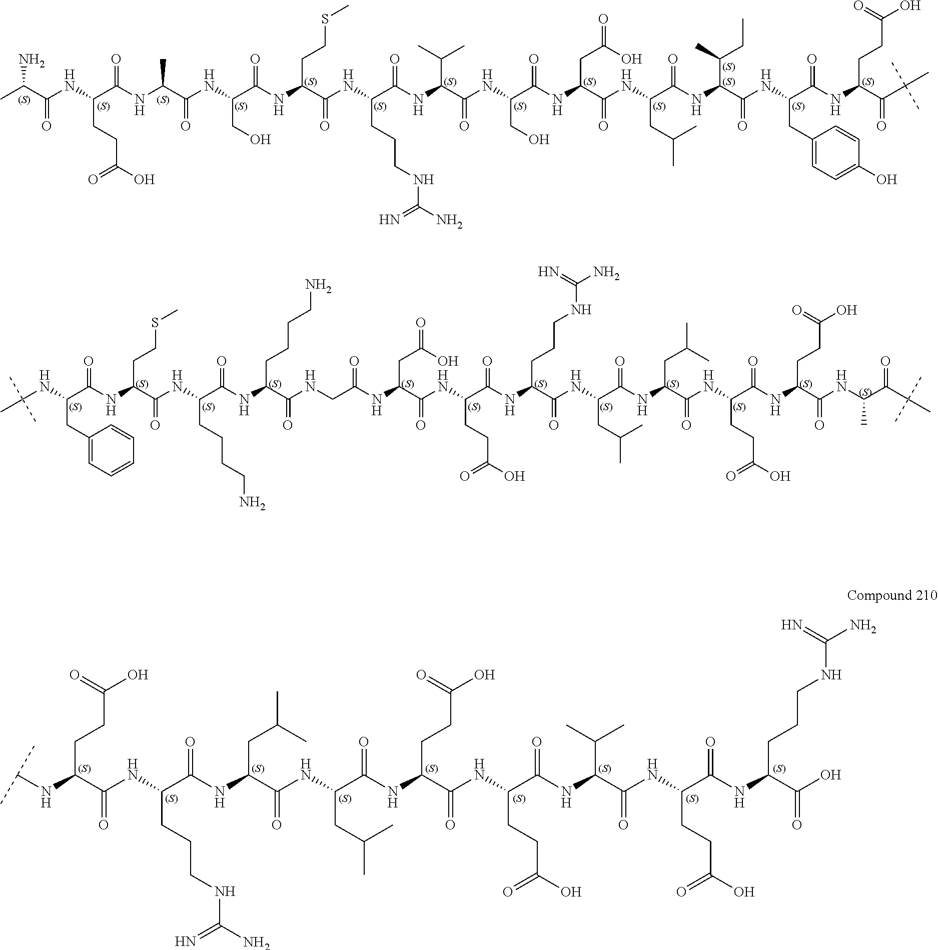

0.20 mmol resin afforded Compound 211 (25.8 mg, 95.0% purity, 2.63% yield) as a white solid. LCMS: RT=1.08 min, MS cal.:4659.27, [M+3H]³⁺=1553.67, [M+4H]⁴⁺=1165.93, [M+5H]⁵⁺=932.95.

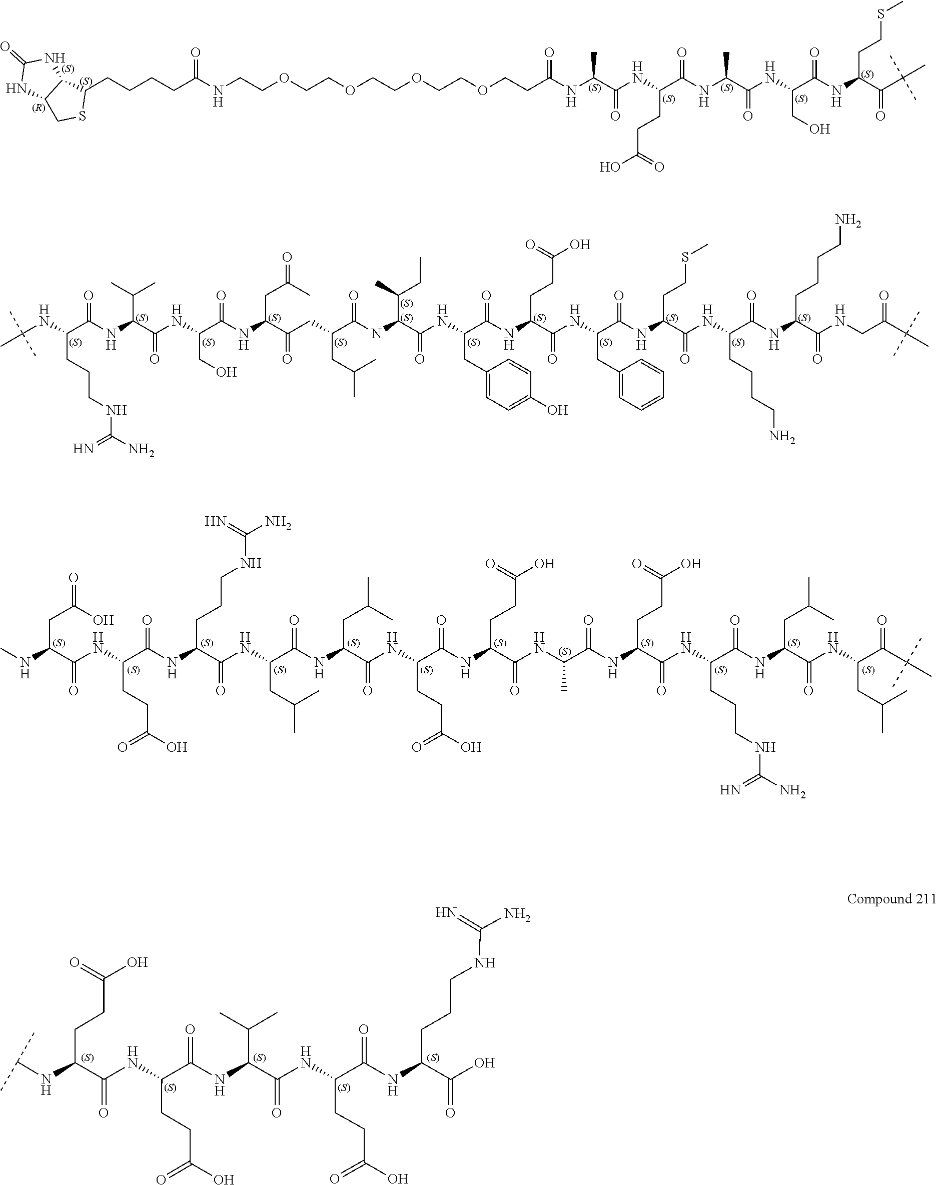

0.20 mmol resin afforded Compound 212 (47.9 mg, 91.5% purity, 2.63% yield) as a white solid. LCMS: RT=1.07 min, MS cal.:5493.21, [M+3H]³⁺=1831.84, [M+4H]⁴⁺=1373.96, [M+5H]⁵⁺=1099.59, [M+7H]⁷⁺=785.66.

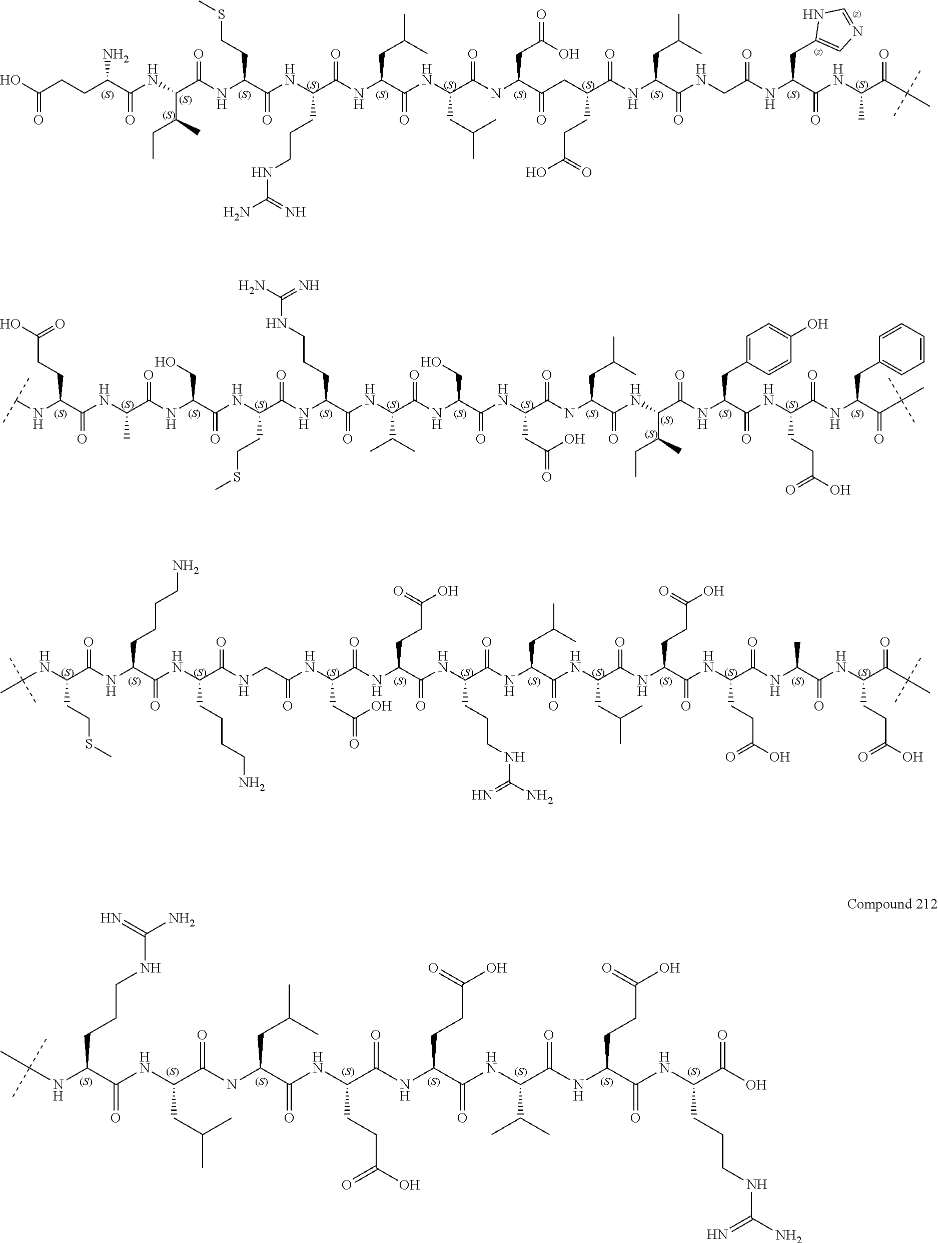

0.20 mmol resin afforded Compound 213 (15.9 mg, 96.1% purity, 1.38% yield) as a white solid. LCMS: RT=1.11 min, MS cal.:5966.79, [M+4H]⁴⁺=1493.17, [M+5H]⁵⁺=1194.24, [M+6H]⁶⁺=995.65, [M+7H]⁷⁺=853.53, [M+8H]⁸⁺=747.06.

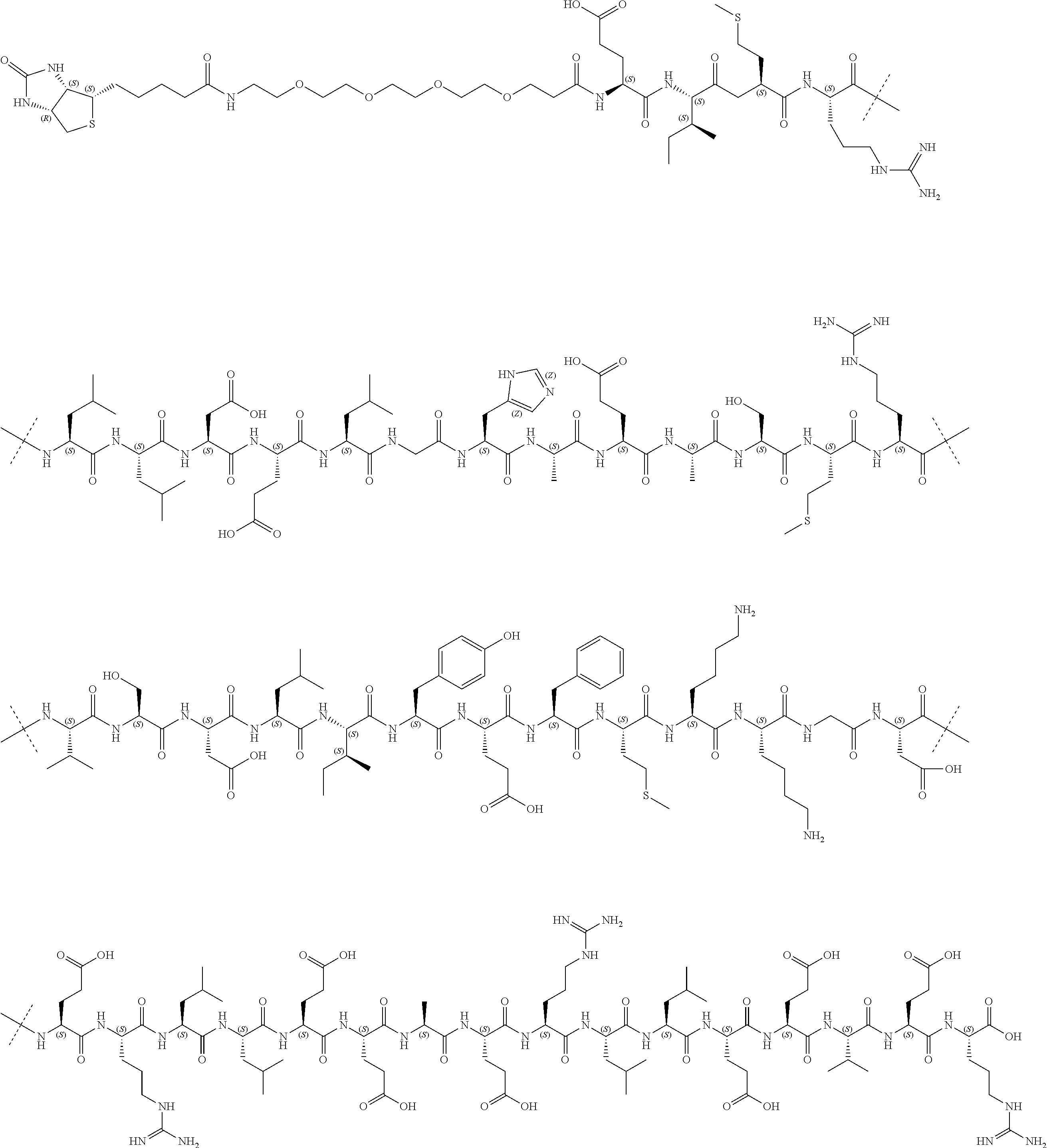

0.20 mmol resin afforded Compound 214 (25.8 mg, 90.0% purity, 1.38% yield) as a white solid. LCMS: RT=1.06 min, MS cal.:5360.25, [M+4H]⁴⁺+=1340.68, [M+5H]⁵⁺=1072.98, [M+6H]⁶⁺=894.20, [M+7H]⁷⁺=766.60, [M+8H]⁸⁺=670.70.

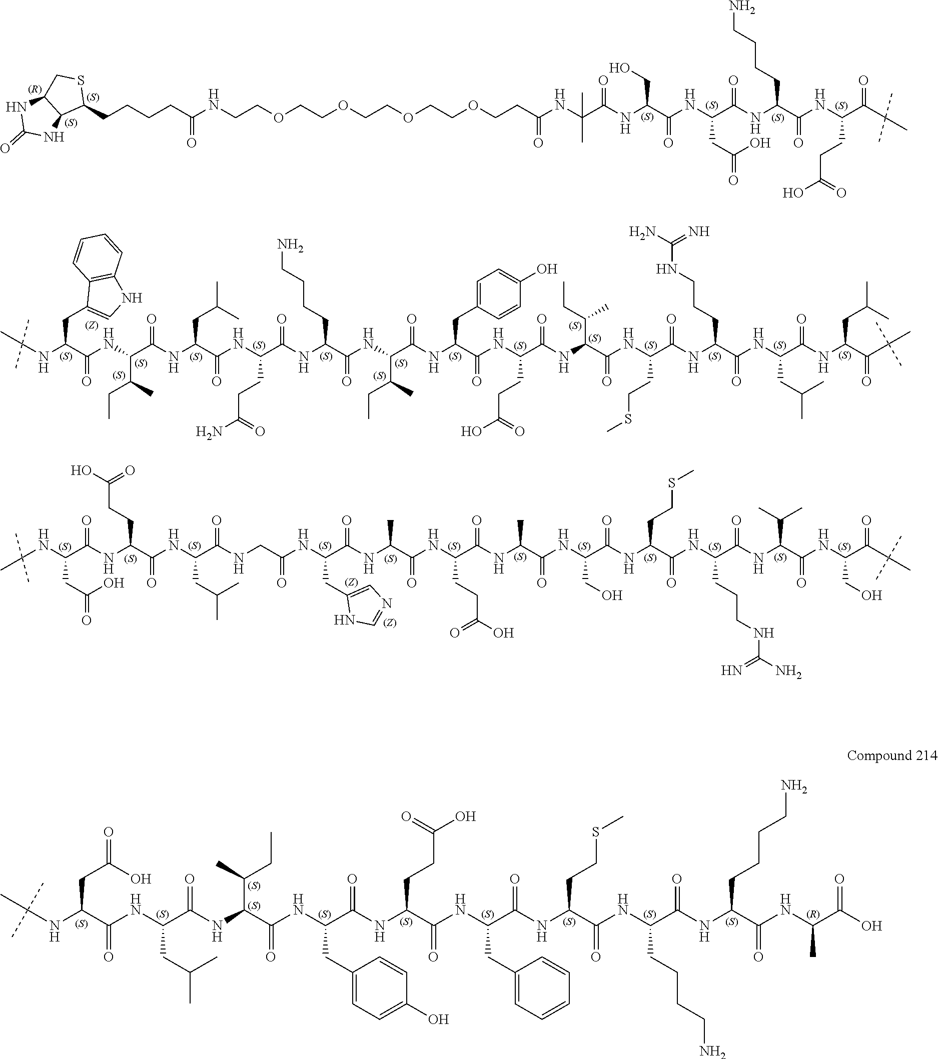

0.20 mmol resin afforded Compound 215 (34.2 mg, 89.1% purity, 3.11% yield) as a white solid. LCMS: RT=1.06 min, MS cal.:4886.67, [M+3H]³⁺=1629.27, [M+4H]⁴⁺+=1222.58, [M+5H]⁵⁺=978.09, [M+6H]⁶⁺=814.96, [M+7H]⁷⁺=699.00.

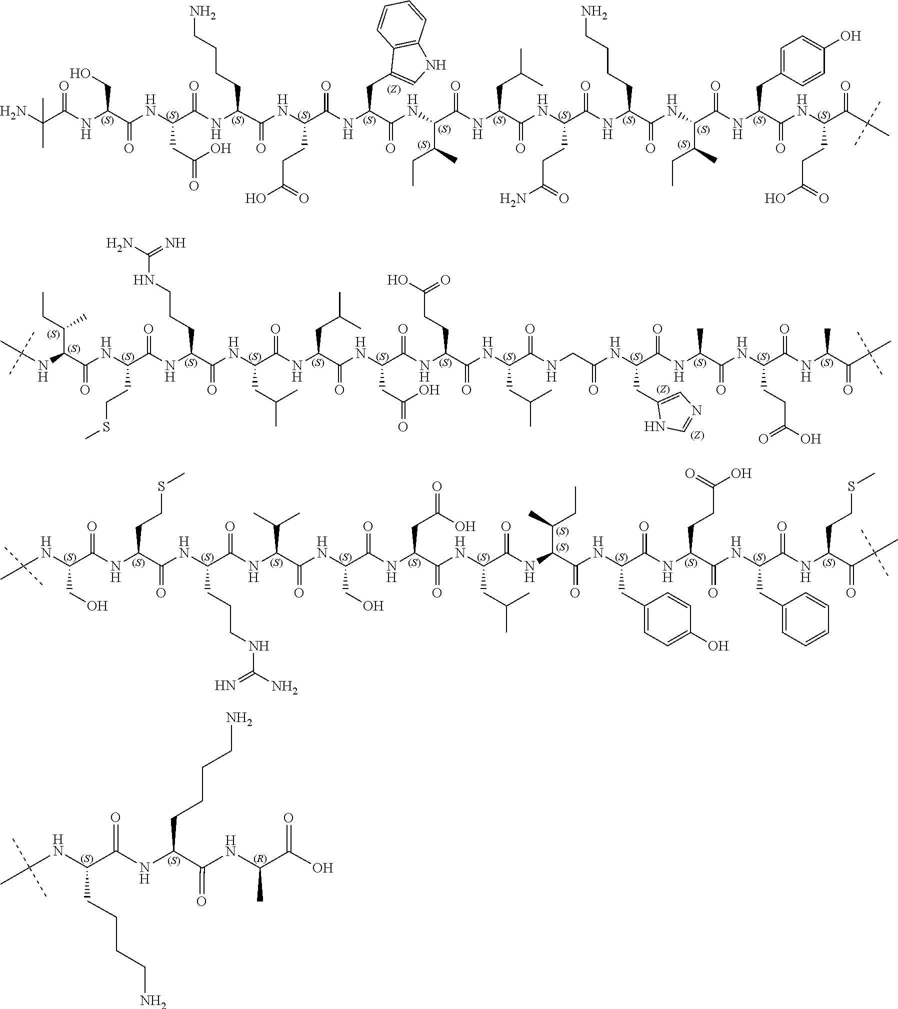

0.20 mmol resin afforded Compound 256 (11.6 mg, 90.1% purity, 1.13% yield) as a white solid. LCMS: RT=1.05 min, MS cal.:4962.76, [M+3H]³⁺=1654.60, [M+4H]⁴⁺=1241.34, [M+5H]⁵⁺=993.33, [M+6H]⁶⁺=827.86.

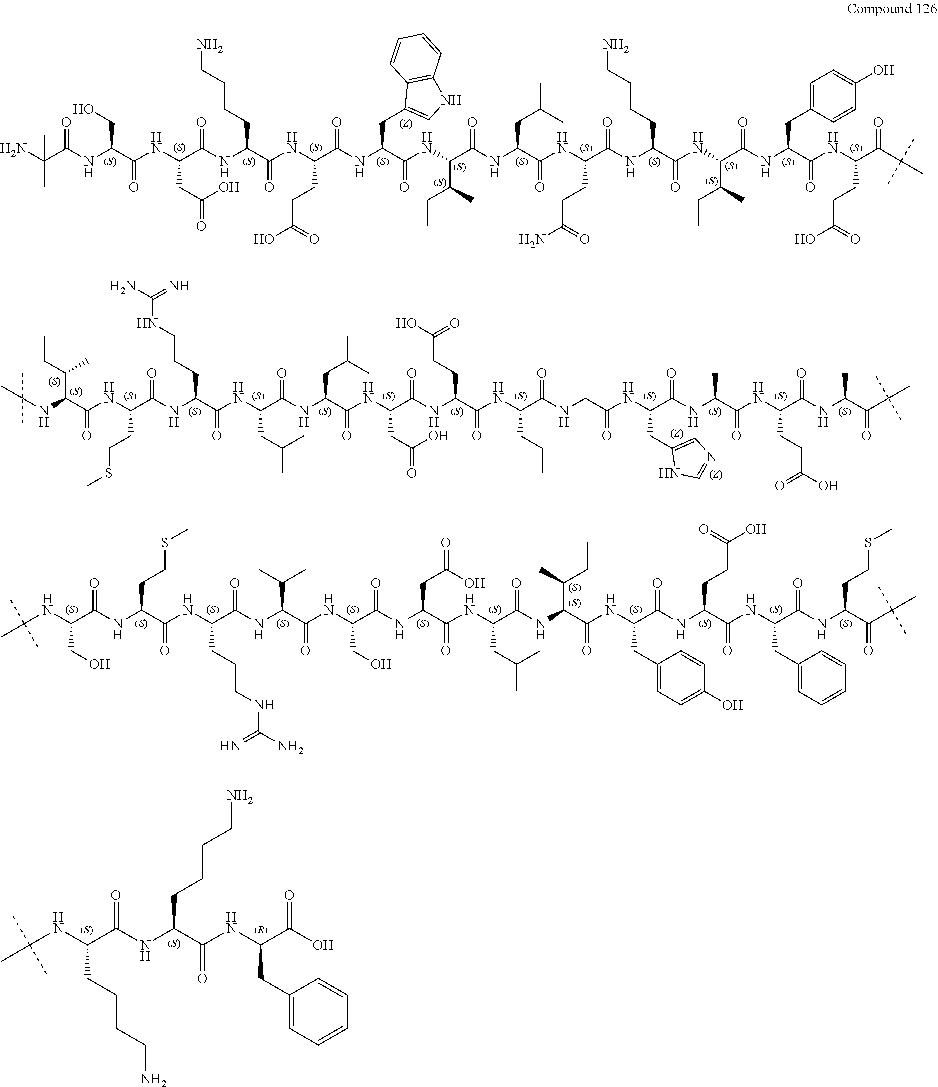

0.20 mmol resin afforded Compound 216 (7.8 mg, 92.3% purity, 0.66% yield) as a white solid. LCMS: RT=1.11 min, MS cal.:5436.35, [M+3H]³⁺=1812.3 7, [M+4H]⁴⁺=1359.80, [M+5H]⁵⁺=1088.00, [M+6H]⁶⁺=907.00, [M+7H]¹¹⁺+=777.47, [M+8H]⁸⁺=680.50.

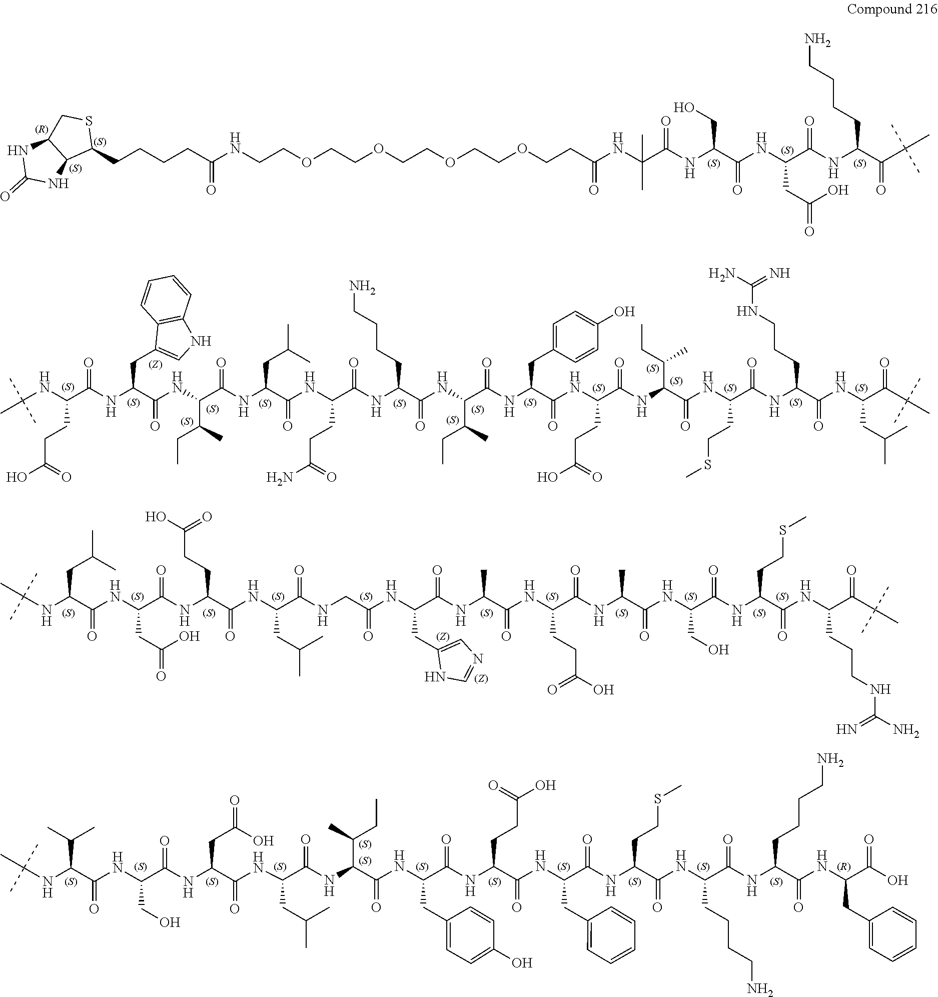

0.20 mmol resin afforded Compound 217 (60.5 mg, 95.2% purity, 6.04% yield) as a white solid. LCMS: RT=1.11 min, MS cal.:4767.52, [M+3H]³⁺=1589.82, [M+4H]⁴⁺=1192.59, [M+5H]⁵⁺=954.40, [M+6H]⁶⁺=795.31.

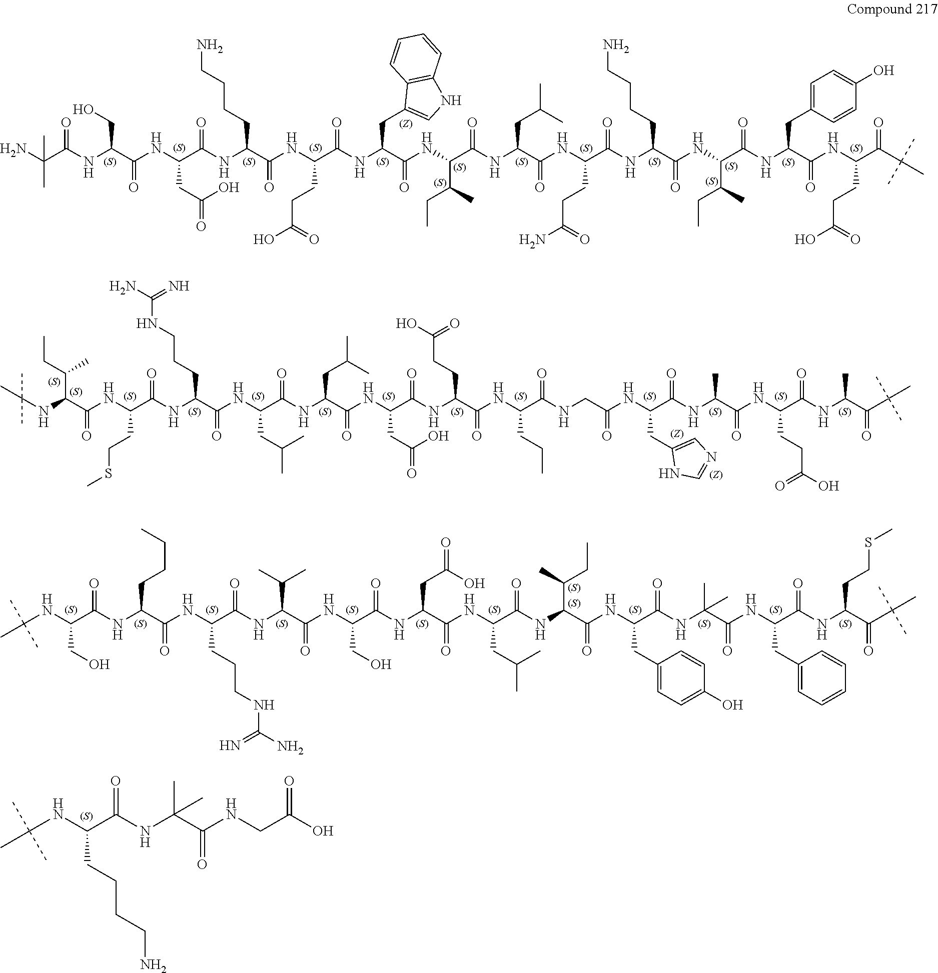

0.20 mmol resin afforded Compound 218 (26.3 mg, 95.1% purity, 2.38% yield) as a white solid. LCMS: RT=1.11 min, MS cal.:5241.11, [M+3H]³⁺=1747.79, [M+4H]⁴⁺=1310.94, [M+5H]⁵⁺=1049.03, [M+6H]⁶⁺=874.26, [M+7H]⁷⁺=794.46.

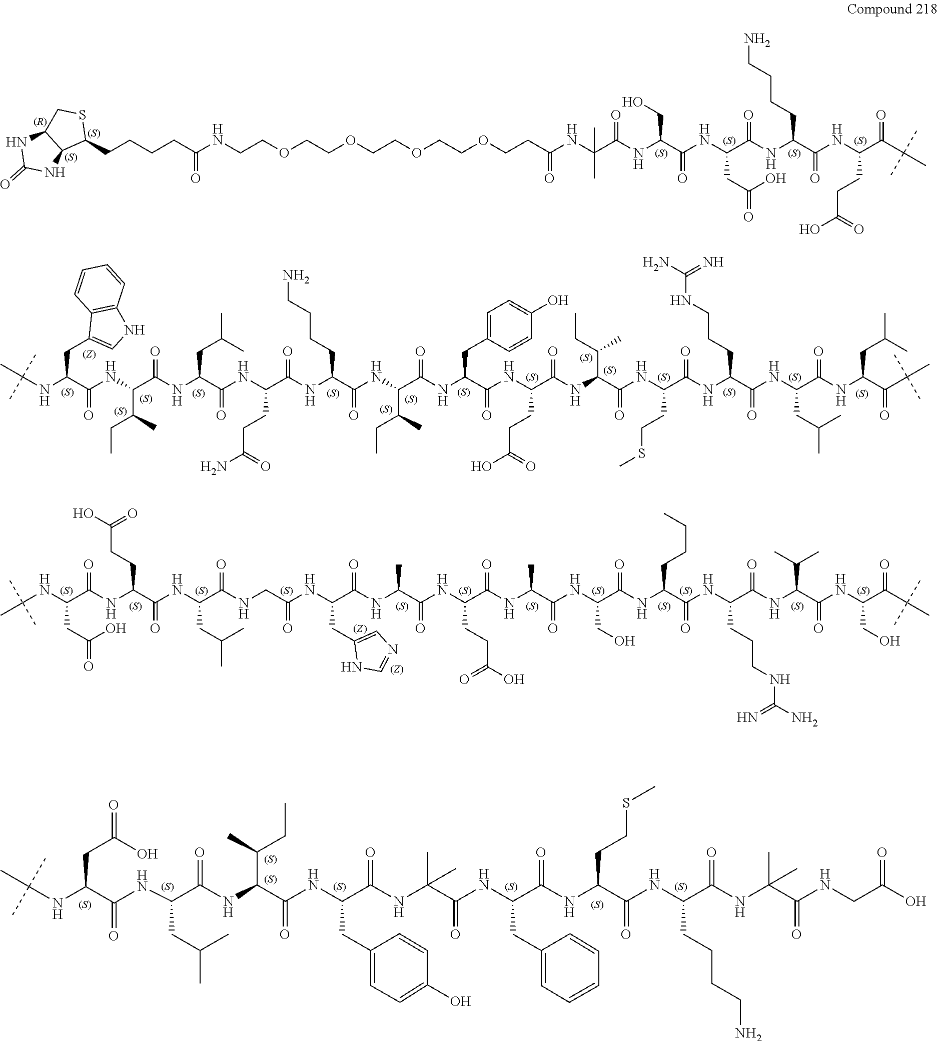

0.20 mmol resin afforded Compound 219 (20.9 mg, 97.4% purity, 2.14% yield) as a white solid. LCMS: RT=1.11 min, MS cal.:4749.49, [M+3H]³⁺=1583.80, [M+4H]⁴⁺+=1188.0 [M+5H]⁵⁺=950.70, [M+6H]⁶⁺=792.30, [M+7H]⁷⁺=679.20.

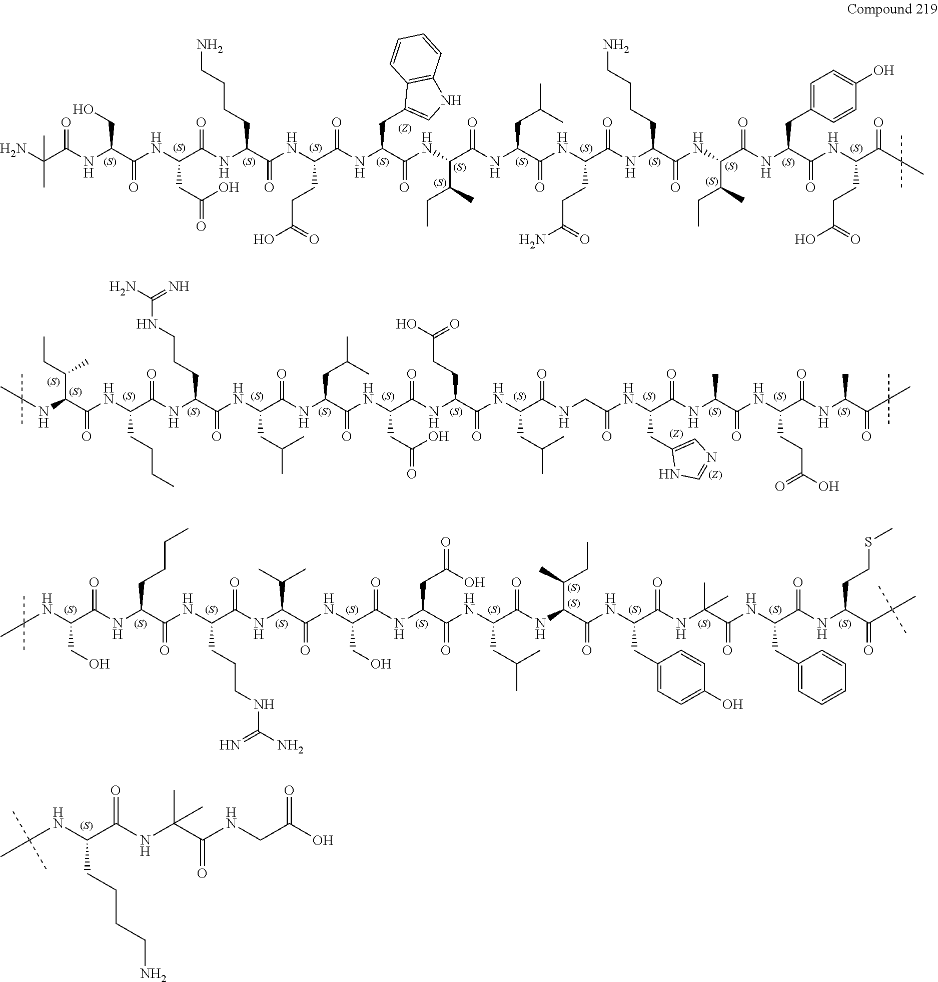

0.20 mmol resin afforded Compound 220 (65.4 mg, 95.7% purity, 5.99% yield) as a white solid. LCMS: RT=1.11 min, MS cal.:5223.07, [M+3H]³⁺=1741.87, [M+4H]⁴⁺=1306.59, [M+5H]⁵⁺=1045.48, [M+6H]⁶⁺=871.31, [M+7H]⁷⁺=746.81.

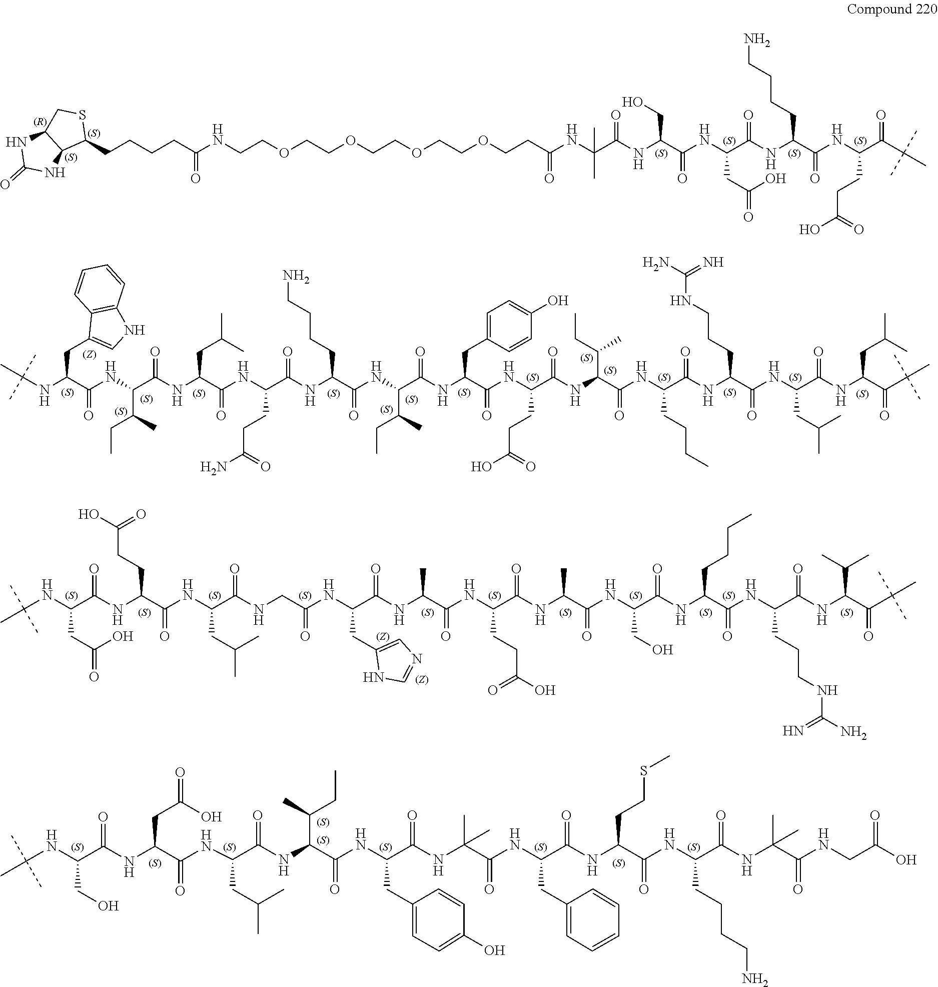

0.20 mmol resin afforded Compound 221 (28 mg, 93.7% purity, 2.74% yield) as a white solid. LCMS: RT=1.00 min, MS cal.:4780.55, [M+3H]³⁺=1594.16, [M+4H]⁴⁺=1195.88, [M+5H]⁵⁺=957.00, [M+6H]⁶⁺=797.48.

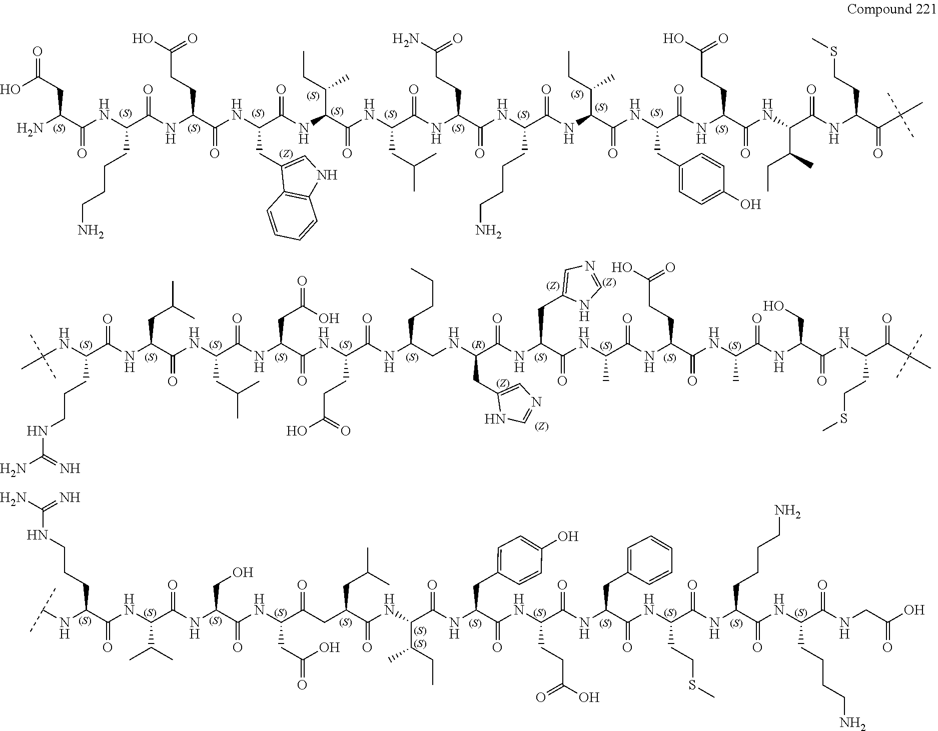

(SEQ ID NO: 94)
H2N-DKEWILQKIYEIMRLLDE[Nle][DHis]HAEASMRVSDLIYEFMK
KG-CO2

0.20 mmol resin afforded Compound 222 (35.2 mg, 90.2% purity, 3.02% yield) as a white solid. LCMS: RT=1.00 min, MS cal.:5254.13, [M+3H]³⁺=1752.29, [M+4H]⁴⁺+=1314.20, [M+5H]⁵⁺=1051.43, [M+6H]⁶⁺=876.50.

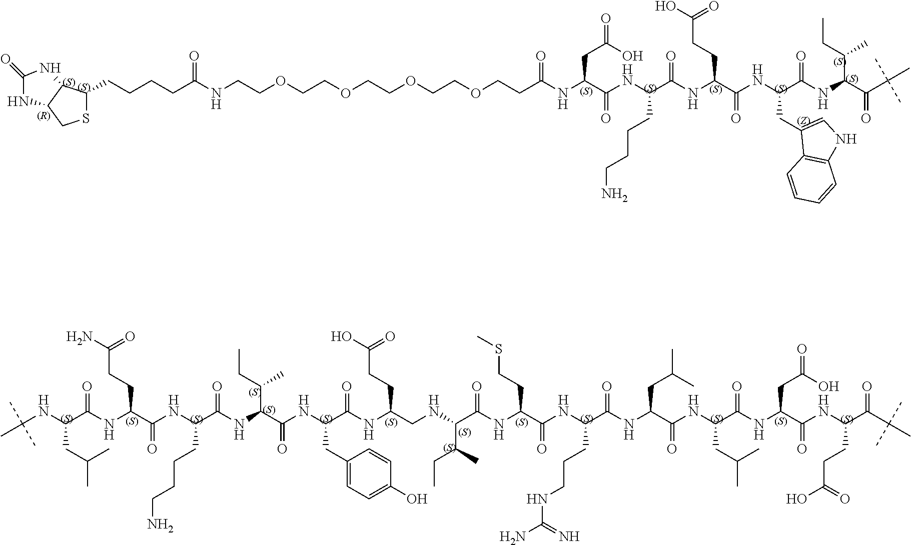

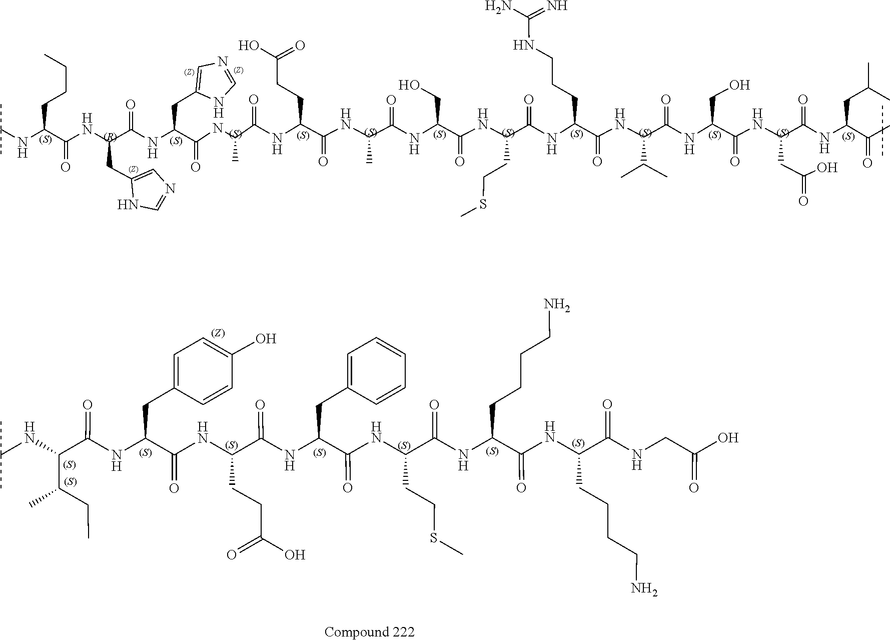

Binder-PEG-DKEWILQKIYEIMRLLDE-Nle-dHis-HAEASMRVSDLIYEFMKKG-CO2 (SEQ ID NO:95)Example 21. Procedure for Preparation of Compound 223 (Linear peptide with Ac)

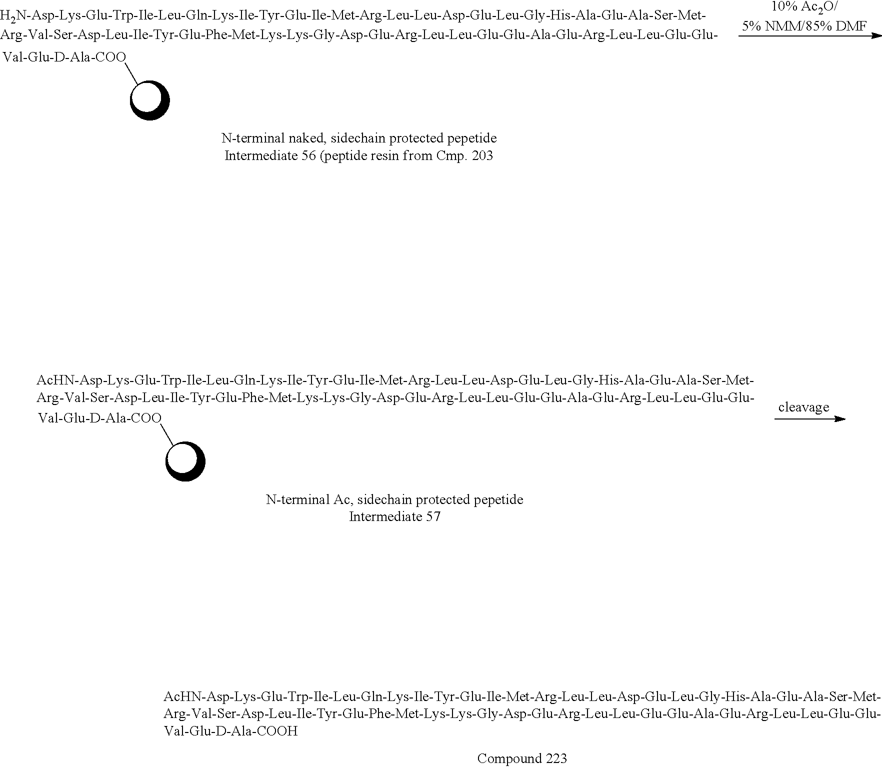

Peptide was synthesized using standard Fmoc chemistry, and SPPS was identical to that for Compound 203, shown in Example 20.

Acetylation: A solution of Ac₂O/NMM/DMF (10/5/85, v/v/v, 30 mL) was added to resin (intermediate 56, 0.1 mmol, 1.20 g) and the mixture was bubbled with N₂ for 20 min. The acetylation reaction was monitored by ninhydrin test, if it showed colorless, the coupling was judged complete. The resin was then washed with DMF (60 mL)*5, MeOH (20 mL)*5, and then dried under vacuum to afford peptide resin intermediate 57 (SEQ ID NO:96) (CTC resin, 0.1 mmol, 1.25 g).

Peptide Cleavage and Purification: Cleavage buffer (TFA/TIS/H₂O/3-mercaptopropanoic acid, 92.5/2.5/2.5/2.5, v/v/v/v, 20 mL) was added to the flask containing the side chain protected peptide resin (intermediate 57, 1.25 g) at room temperature and stirred for 2 h.

The filtrate was collected. The peptide was precipitated with cold isopropyl ether (100 mL) and centrifuged (3 min at 3000 rpm). Isopropyl ether washed two additional times, and the crude peptide was dried under vacuum for 2 h to afford Compound 223 (SEQ ID NO:96) (0.83 g, crude) as a white solid.

The crude was purified by prep-HPLC (acid condition, TFA), followed by lyophilization to afford Compound 223 (18.7 mg, 84.9% purity, 2.34% yield) as a white solid. LCMS: RT=1.15 min, MS cal.: 6767.81, [M+5H]⁵⁺=1354.32, [M+6H]⁶⁺=1128.67, [M+7H]⁷⁺=967.45, [M+8H]⁸⁺=846.45, [M+9H]⁹⁺=752.84.

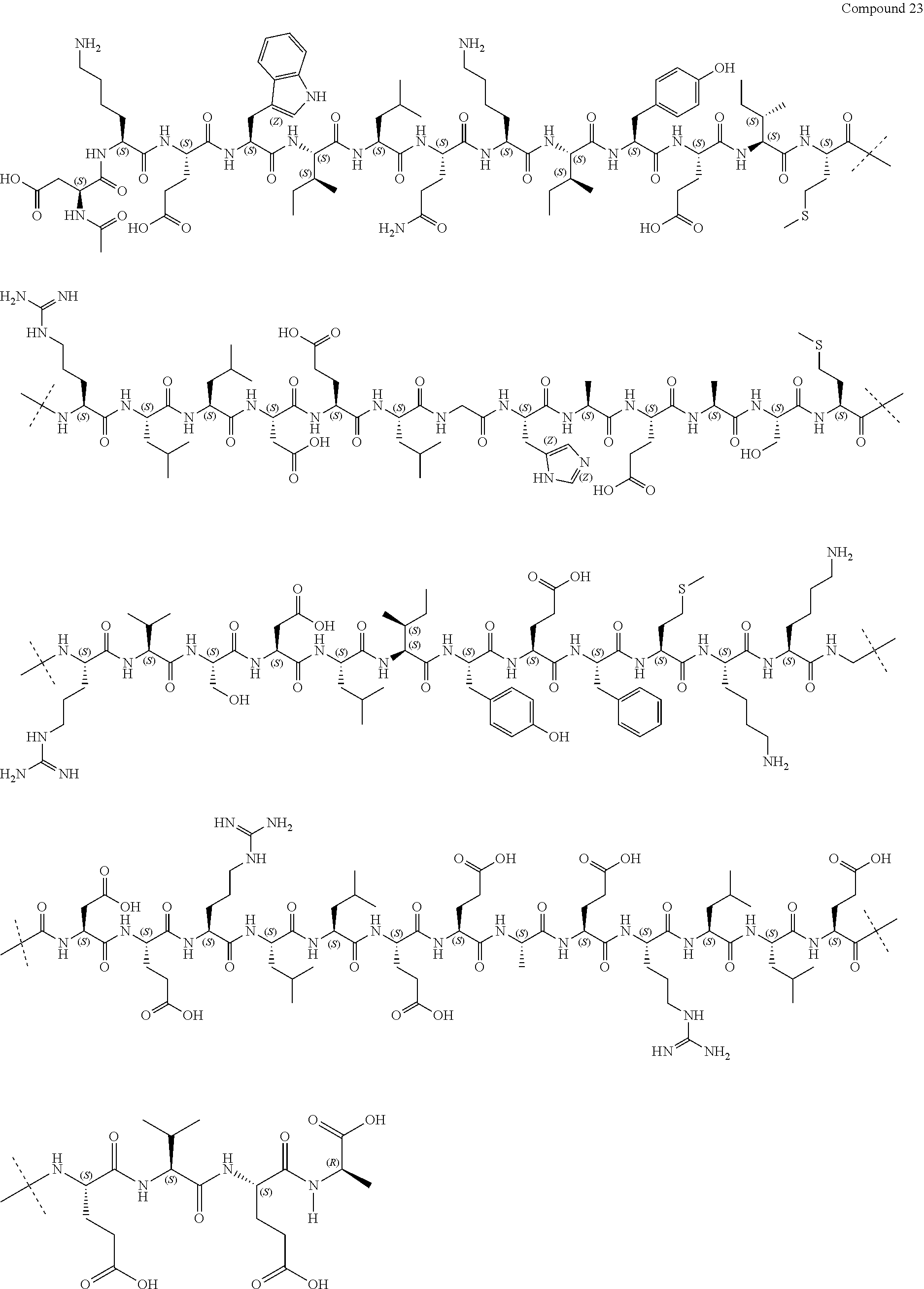

(SEQ ID NO: 97)
Ace-DKEWILQKIYEIMRLLDELGHAPASMRVSDLIYEFMKKGDERLLEE
AERLLEEVE[DAla]

Compounds 224, 225, 254, 226, 227, 228, 229, 230, 231, 232, 103, 233, 234, 235, 236, 237, 238, 239, and 240 were synthesized using the same method as Compound 223, Compound 255.

0.10 mmol resin afforded Compound 224 obtains (10.2 mg, 91.9% purity, 1.38% yield) as a white solid. LCMS: RT=1.16 min, MS cal.: 6781.84, [M+5H]⁵⁺=1357.14, [M+6H]⁶⁺=1130.66, [M+7H]⁷⁺=969.43, [M+9H]⁹⁺=753.94.

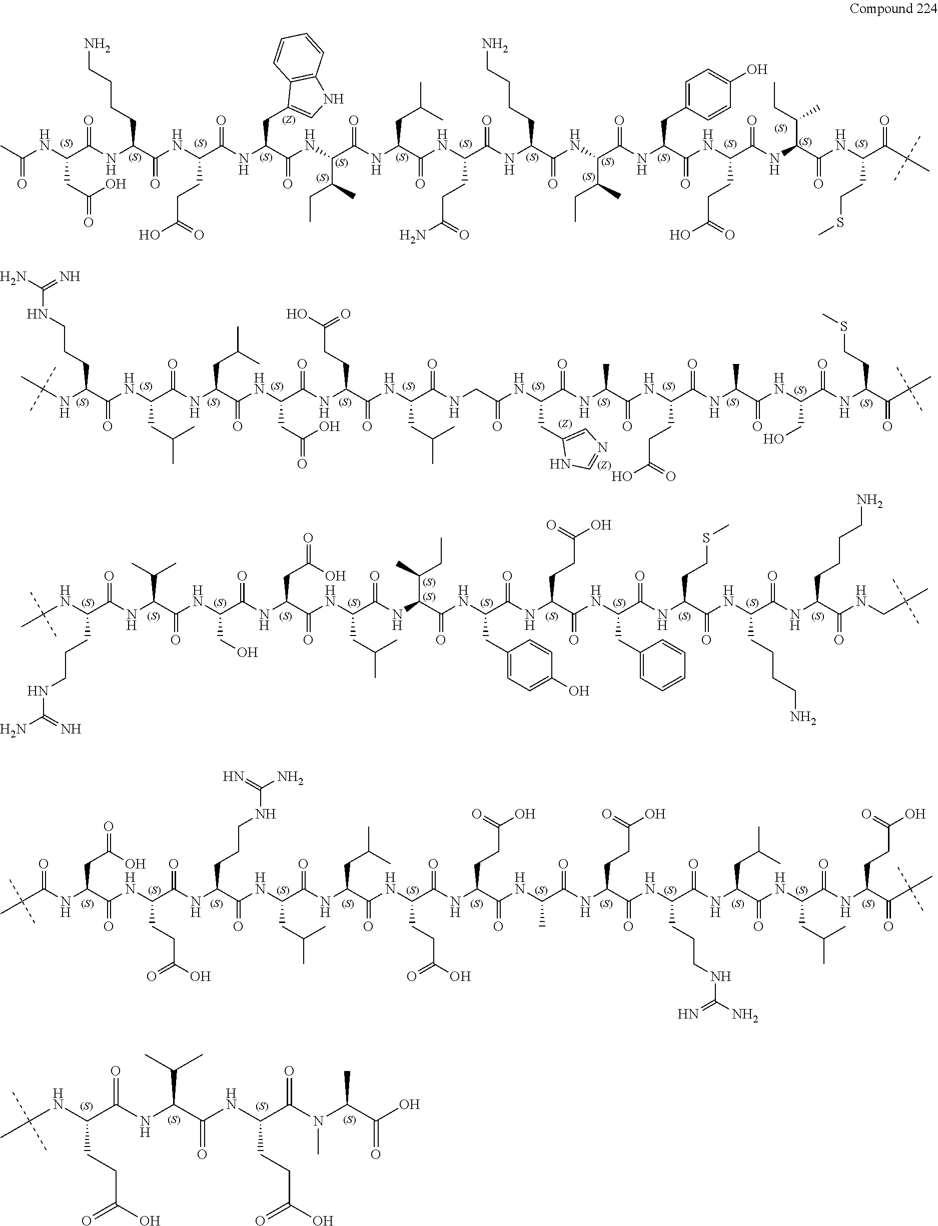

0.50 mmol resin afforded Compound 225 (14.6 mg, 86.4% purity, 0.43% yield) as a white solid. LCMS: RT=1.14 min, MS cal.: 6852.78, [M+6H]⁶⁺=1142.85, [M+7H]⁷⁺=980.10.

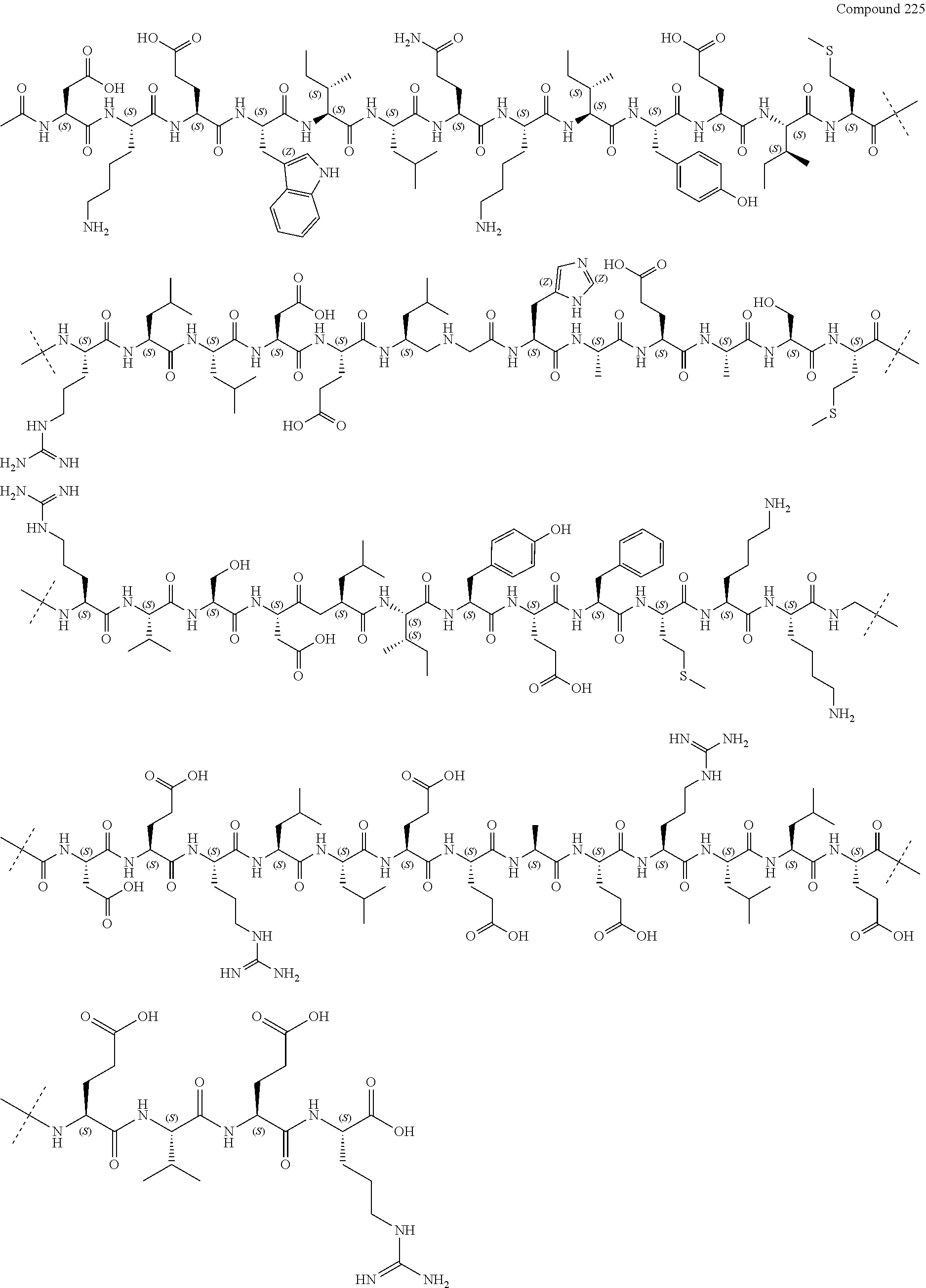

0.50 mmol resin afforded Compound 254, 24.8 mg, 91.1% purity, 0.68% yield) as a white solid. LCMS: RT=1.09 min, MS cal.: 7276.27, [M+4H]⁴⁺+=1819.50, [M+5H]⁺=1456.20, [M+6H]⁶⁺=1213.58, [M+7H]⁷⁺=1040.30, [M+8H]⁸⁺=910.35, [M+9H]⁹⁺=809.20, [M+10H]¹⁰⁺=728.38.

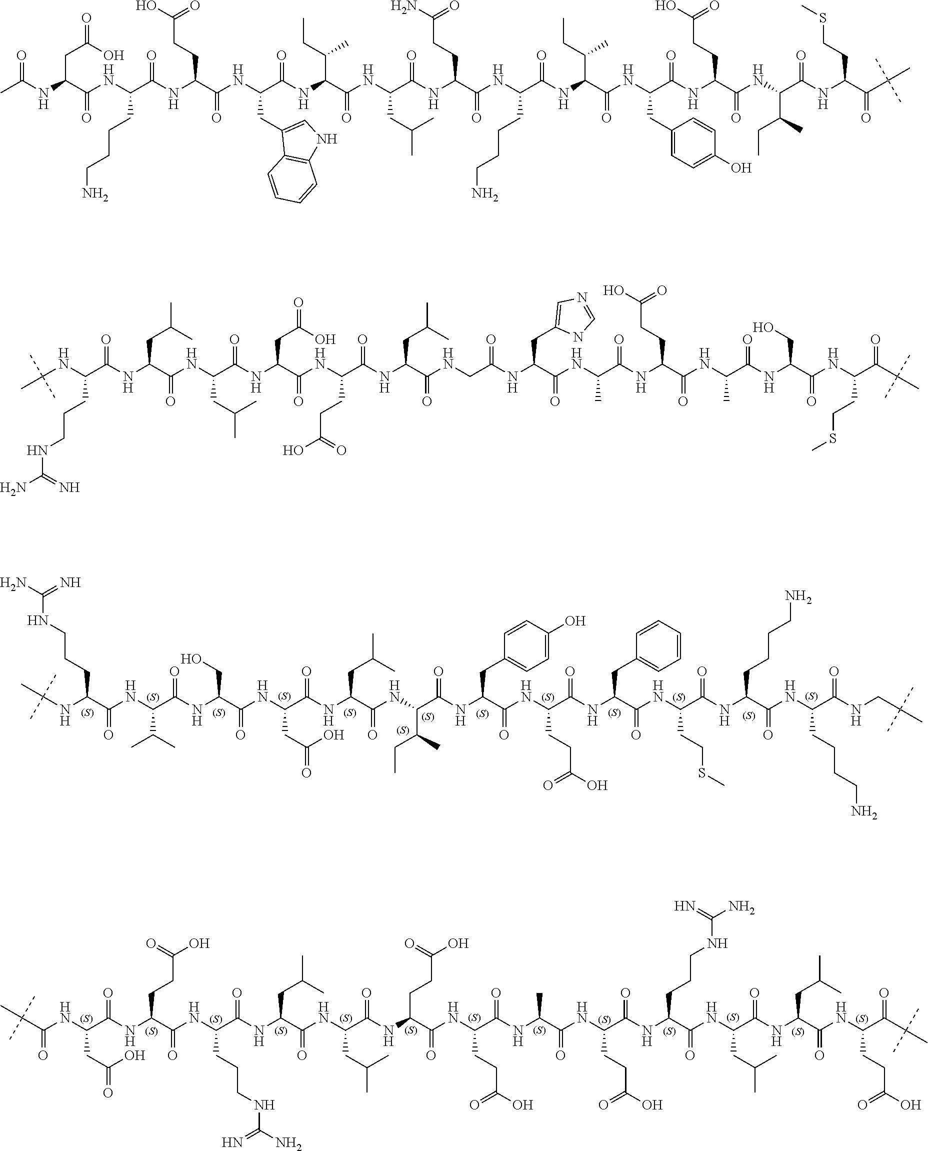

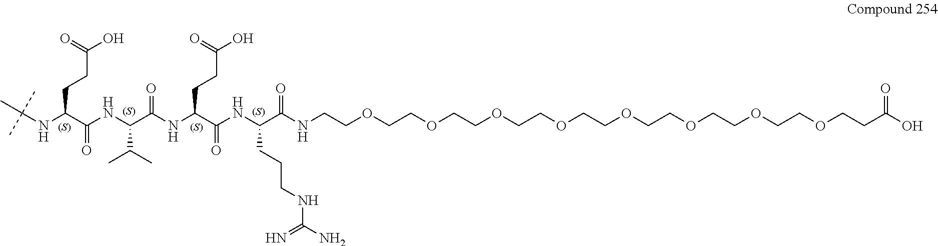

0.50 mmol resin afforded Compound 226 (50.5 mg, 88.8% purity, 1.96% yield) as a white solid. LCMS: RT=1.05 min, MS cal.: 5165.99, [M+3H]³⁺=1722.97, [M+4H]⁴⁺+=1292.40, [M+5H]⁵⁺=1034.02, [M+6H]⁶⁺=861.80, [M+7H]⁷⁺=738.89, [M+8H]⁸⁺=646.57. (This compound matches 2604, but the MS spec does not.)

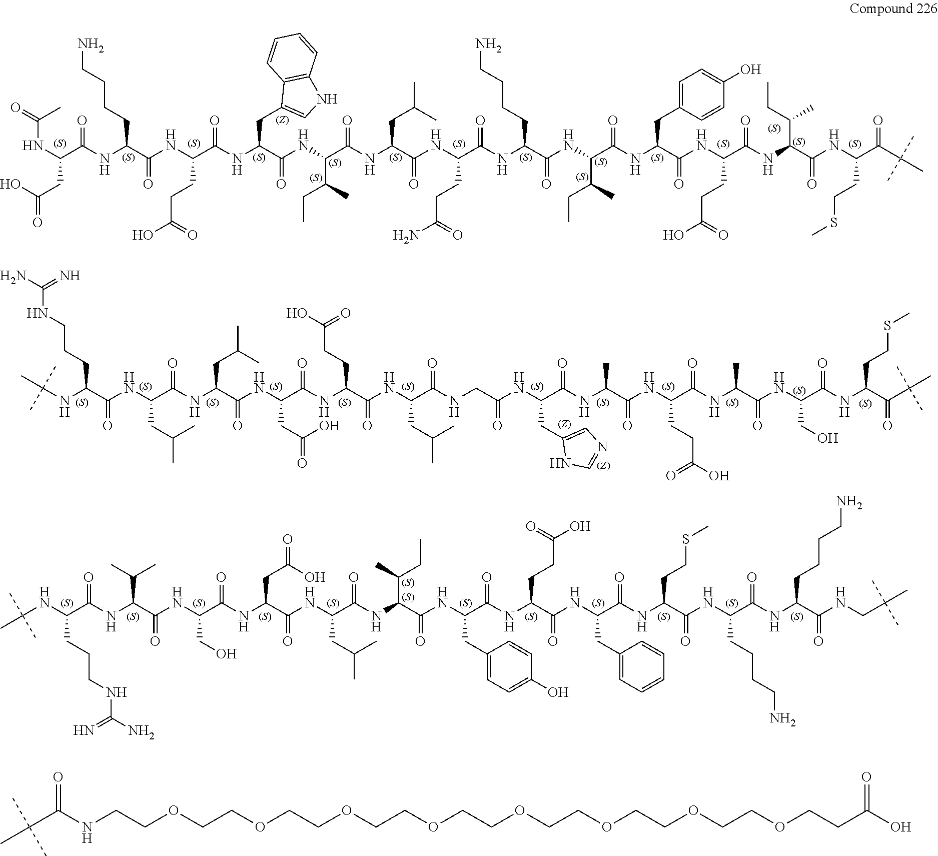

(SEQ ID NO: 98)
Ac-DKEWILQKIYEIMRLLDELGHAEASMRVSDLIYEFMKKG-PEG8-
COOH

0.50 mmol resin afforded Compound 227 (25.4 mg, 93.8% purity, 0.97% yield) as a white solid. LCMS: RT=1.06 min, MS cal.: 5251.10, [M+4H]⁴⁺+=1313.66, [M+5H]⁵⁺=1051.13, [M+6H]⁶⁺=876.00, [M+7H]⁷⁺=750.87, [M+8H]⁸⁺=657.21.

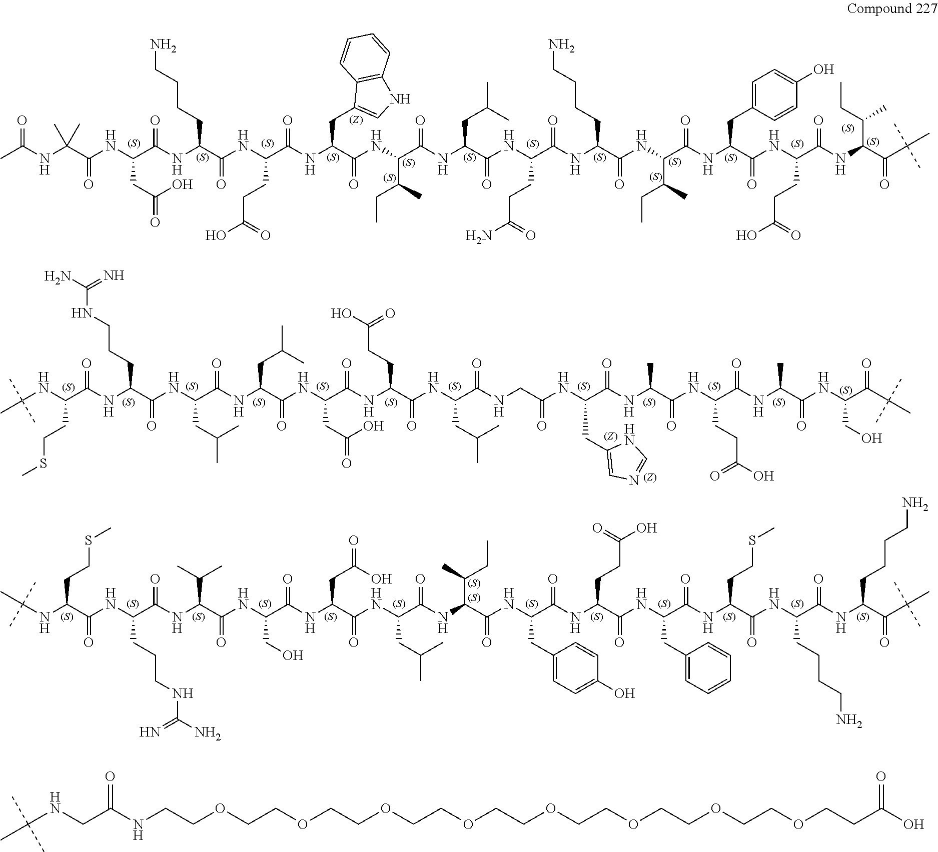

(SEQ ID NO: 99)
Ac-Aib-DKEWILQKIYEIMRLLDELGHAEASMRVSDLIYEFMKKG-
PEG8-COOH

0.50 mmol resin afforded Compound 228 (123.9 mg, 96.8% purity, 8.34% yield) as a white solid. LCMS: RT=1.06 min, MS cal.: 2971.46, [M+2H]²⁺=1486.41, [M+3H]³⁺=991.30, [M+4H]⁴⁺+=743.52, [M+5H]⁵⁺=595.18.

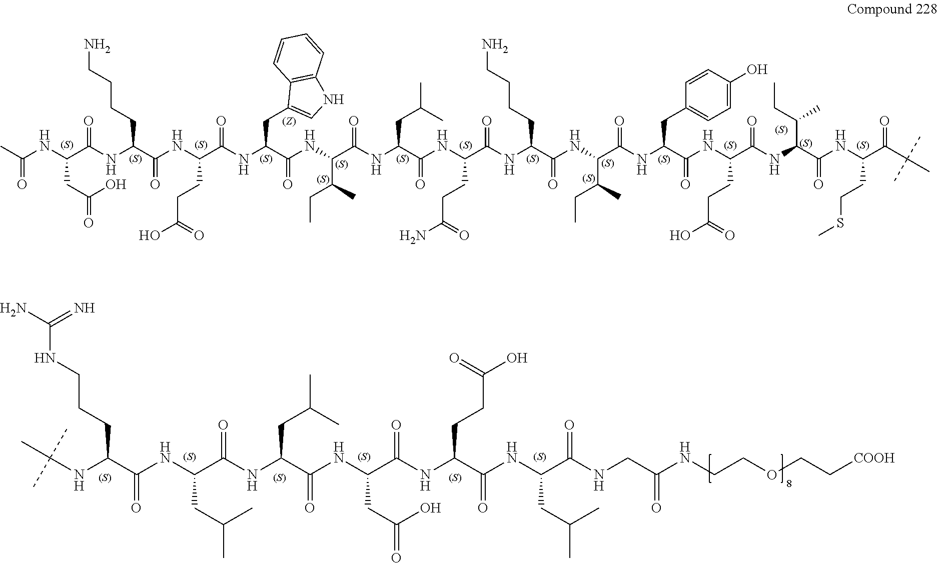

(SEQ ID NO: 100)
Ac-DKEWILQKIYEIMRLLDELG-PEG8-COOH

0.50 mmol resin afforded Compound 229 (146.3 mg, 99.0% purity, 8.44% yield) as a white solid. LCMS: RT=1.02 min, MS cal.: 3466.95, [M+2H]²⁺=1734.50, [M+3H]³⁺=1156.50, [M+4H]⁴⁺+=867.47, [M+5H]⁵⁺=694.10.

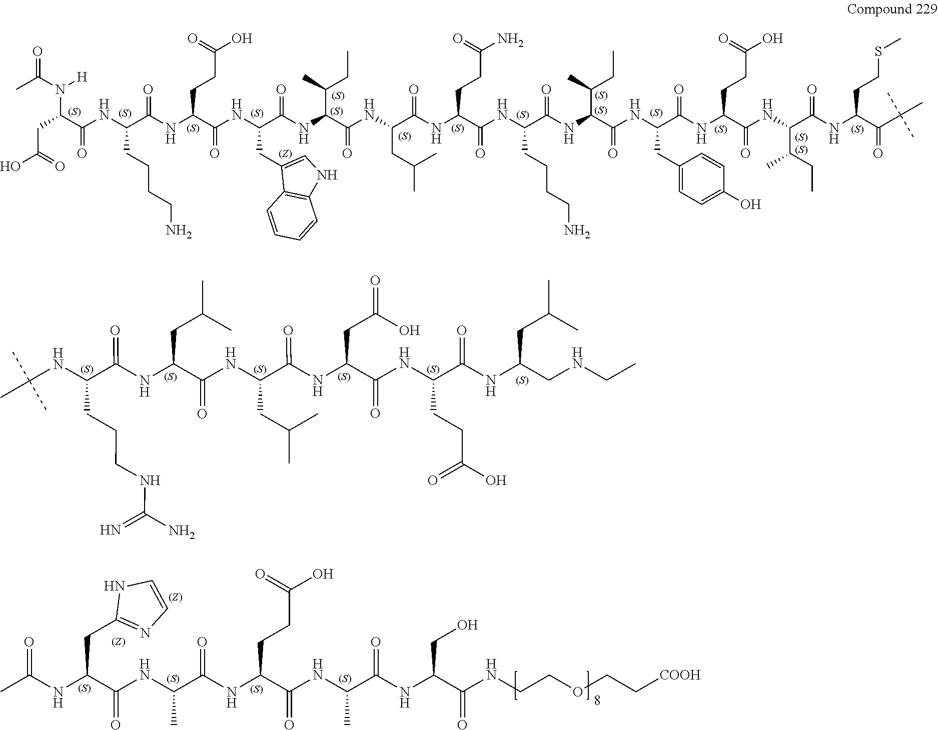

(SEQ ID NO: 101)
Ac-DKEWILQKIYEIMRLLDELGHAEASMRVSDLIYEFMKKG-PEG8-
COOH

0.50 mmol resin afforded Compound 230 (Batch1: 46.1 mg, 90.1% purity; batch2: 59.6 mg, 93.7% purity, 5.37% yield) as a white solid. LCMS: RT=1.01 min, MS cal.: 3940.54, [M+2H]²⁺=1970.68, [M+3H]³⁺=1314.03, [M+4H]⁴⁺+=985.78, [M+5H]⁵⁺=788.60, [M+6H]⁶⁺=657.40.

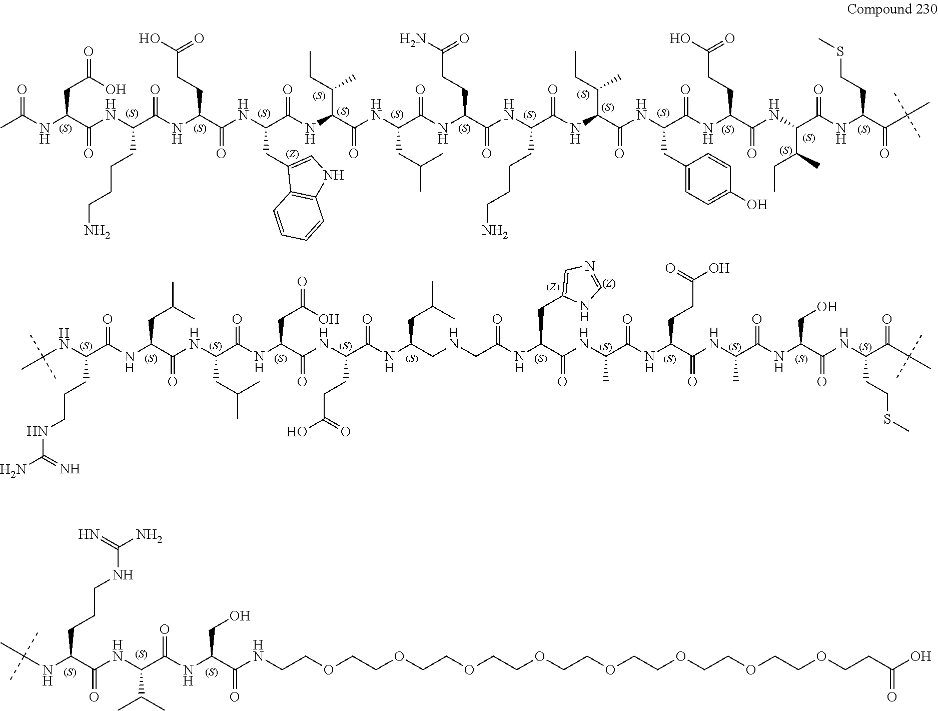

(SEQ ID NO: 102)
Ac-DKEWILQKIYEIMRLLDELGHAEASMRVS-PEG8-COOH

0.50 mmol resin afforded Compound 231 (Batch1: 52.6 mg, 93.9% purity; batch2: 42.6 mg, 93.8% purity, 4.16% yield) as a white solid. LCMS: RT=1.02 min, MS cal.: 4574.23, [M+3H]³⁺=1525.51, [M+4H]⁴⁺+=1144.33, [M+5H]⁵⁺=915.50, [M+6H]⁶⁺=763.20, [M+7H]⁷⁺=654.30.

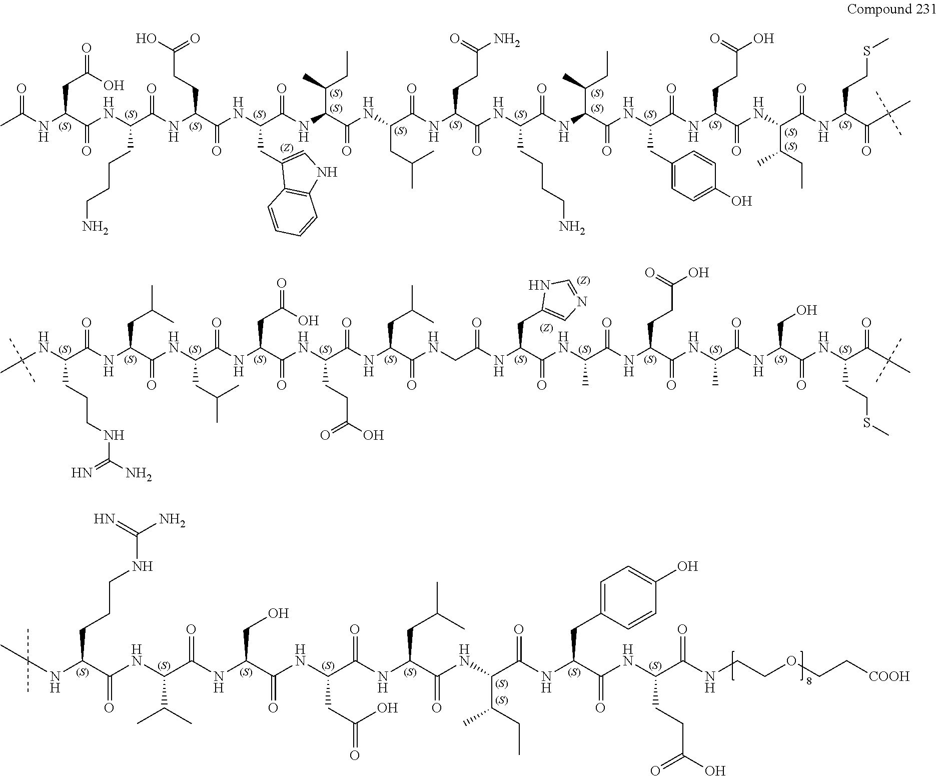

0.50 mmol resin afforded Compound 232 (35.8 mg, 79.4% purity, 1.55% yield) as a white solid. LCMS: RT=1.08 min, MS cal.: 4607.41, [M+3H]³⁺=1536.50, [M+4H]⁴⁺=1152.53, [M+5H]⁵⁺=922.33, [M+6H]⁶⁺=768.63, [M+7H]⁷⁺=659.00.

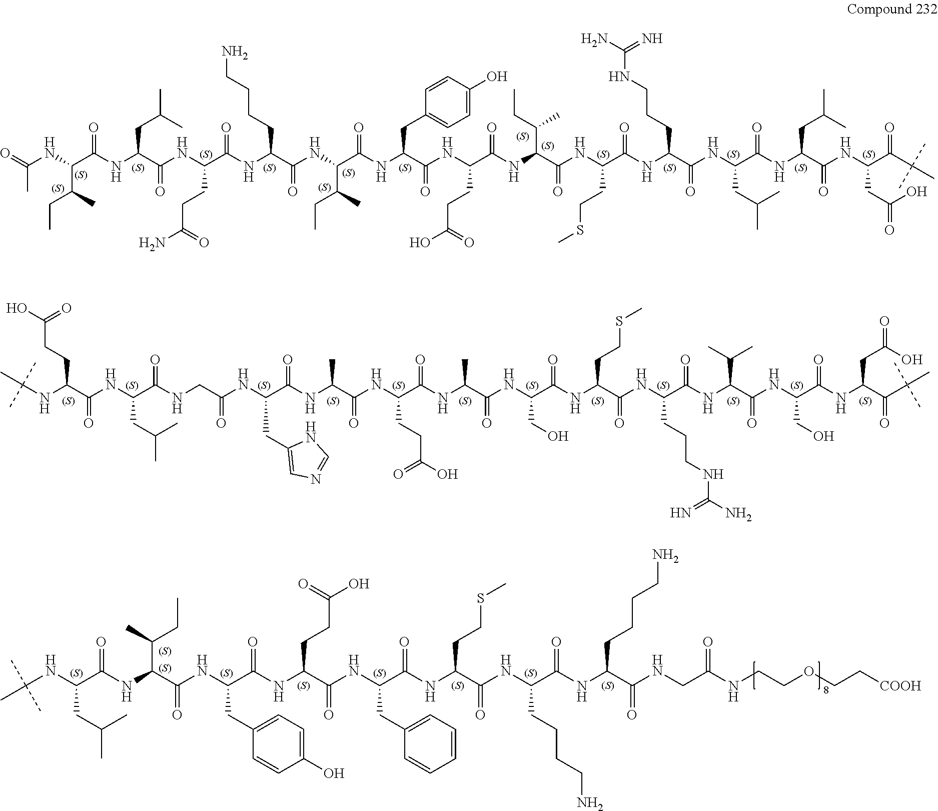

(SEQ ID NO: 103)
Ac-ILQKIYEIMRLLDELGHAEASMRVSDLIYEFMKKG-PEG8-COOH

0.50 mmol resin afforded Compound 233 (96.2 mg, 96.5% purity, 4.66% yield) as a white solid. LCMS: RT=1.08 min, MS cal.: 4124.79, [M+3H]³⁺=1375.66, [M+4H]⁴⁺=1032.02, [M+5H]⁵⁺=825.74, [M+6H]⁶⁺=688.30, [M+7H]⁷⁺=590.00.

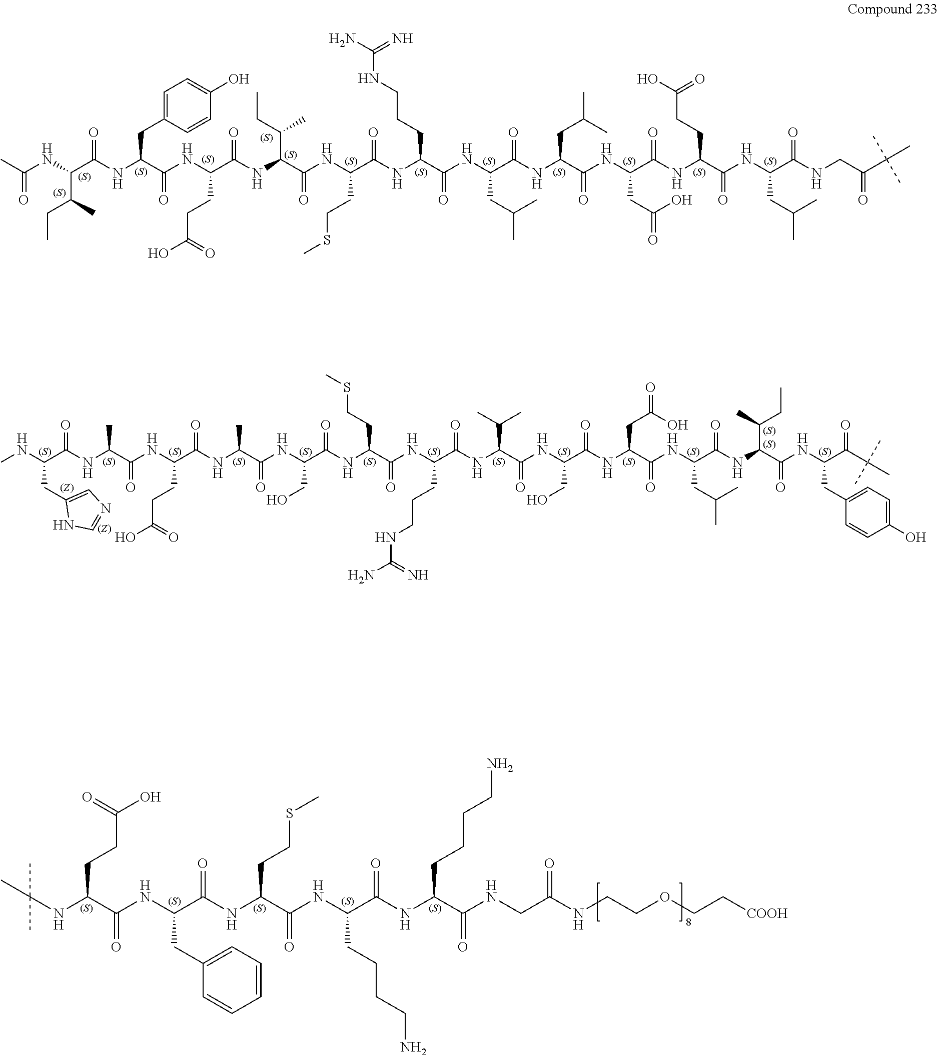

(SEQ ID NO: 104)
Ac-IYEIMRLLDELGHAEASMRVSDLIYEFMKKG-PEG8-COOH

0.20 mmol resin afforded Compound 103 (32.8 mg, 91.8% purity, 3.17% yield) as a white solid. LCMS: RT=1.08 min, MS cal.:4742.49, [M+3H]³⁺=1581.47, [M+4H]⁴⁺=1186.47, [M+5H]⁵⁺=949.23, [M+6H]⁶⁺=704.96.

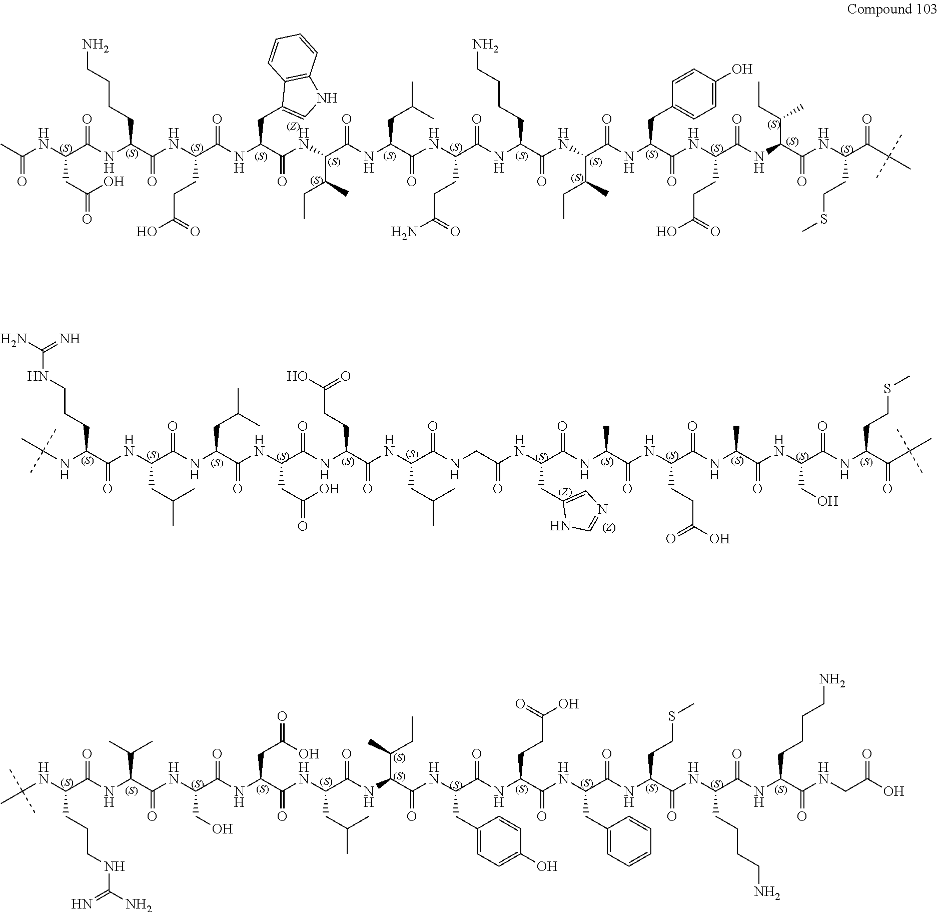

0.20 mmol resin afforded Compound 234 (69.4 mg, 95.5% purity, 7.84% yield) as a white solid. LCMS: RT=1.08 min, MS cal.:4227.73, [M+3H]³⁺=1049.99, [M+4H]⁴⁺+=1057.65, [M+5H]⁵⁺=846.53.

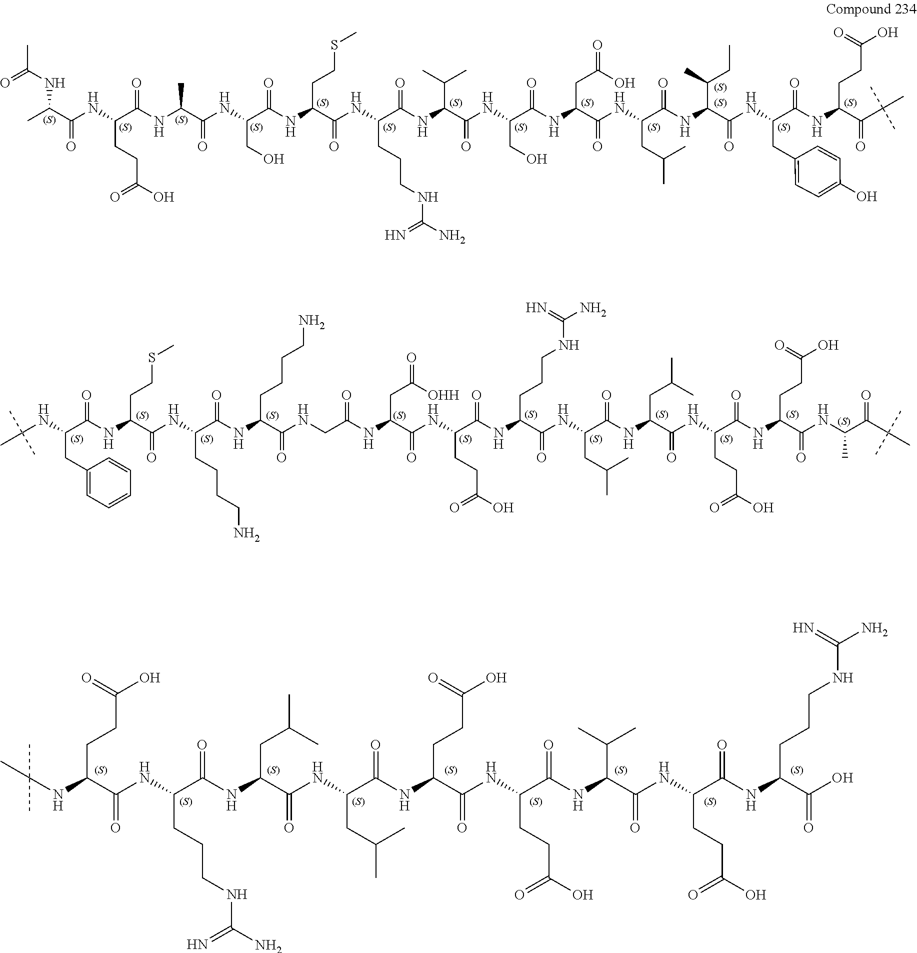

0.20 mmol resin afforded Compound 235 (92.0 mg, 93.7% purity, 7.78% yield) as a white solid. LCMS: RT=1.11 min, MS cal.:5535.24, [M+3H]³⁺=1846.25, [M+4H]⁴⁺=1384.65, [M+5H]⁵⁺=1107.96, [M+7H]⁷⁺=791.82.

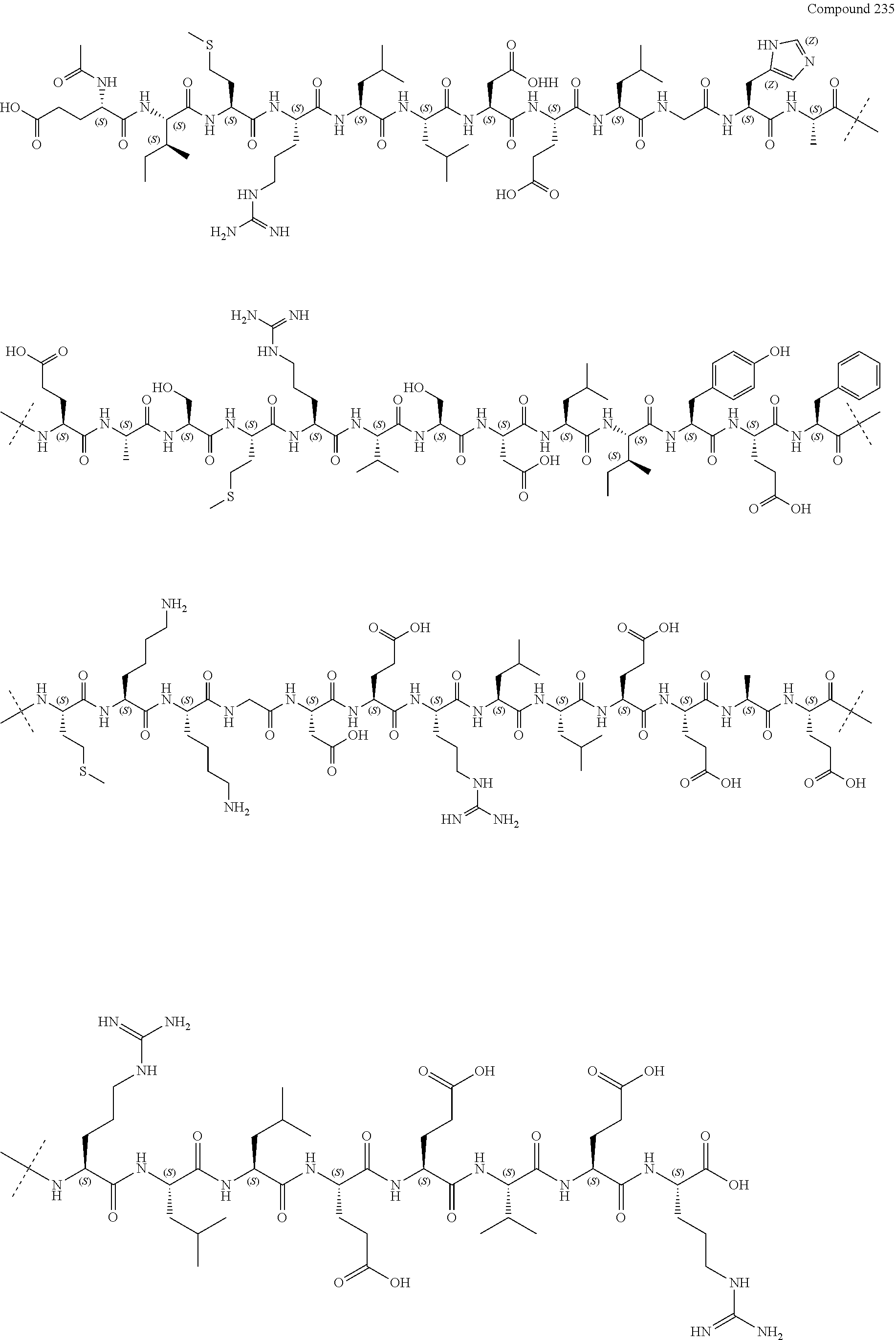

0.20 mmol resin afforded Compound 236 (23.3 mg, 90.1% purity, 3.11% yield) as a white solid. LCMS: RT=1.05 min, MS cal.:4928.70, [M+3H]³⁺=1643.40, [M+4H]⁴⁺=1233.00, [M+5H]⁵⁺=986.41, [M+6H]⁶⁺=822.10, [M+7H]⁷⁺=704.93.

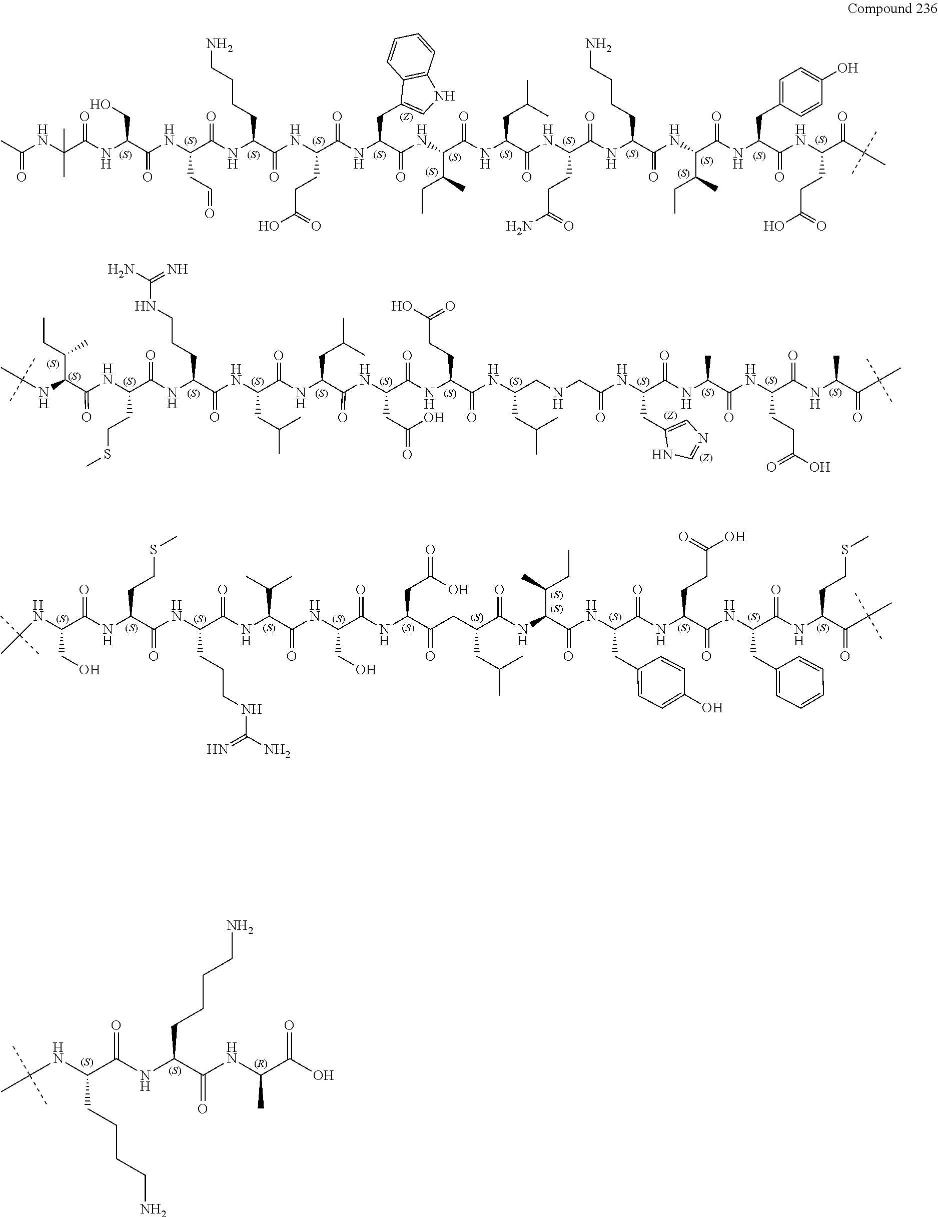

0.20 mmol resin afforded Compound 237 (12.8 mg, 84.5% purity, 1.08% yield) as a white solid. LCMS: RT=1.11 min, MS cal.:5004.80, [M+3H]³⁺=1668.95, [M+4H]⁴⁺=1251.90, [M+5H]⁵⁺=1001.56, [M+6H]⁶⁺=834.90, [M+7H]⁷⁺=715.68, [M+8H]⁸⁺=626.43.

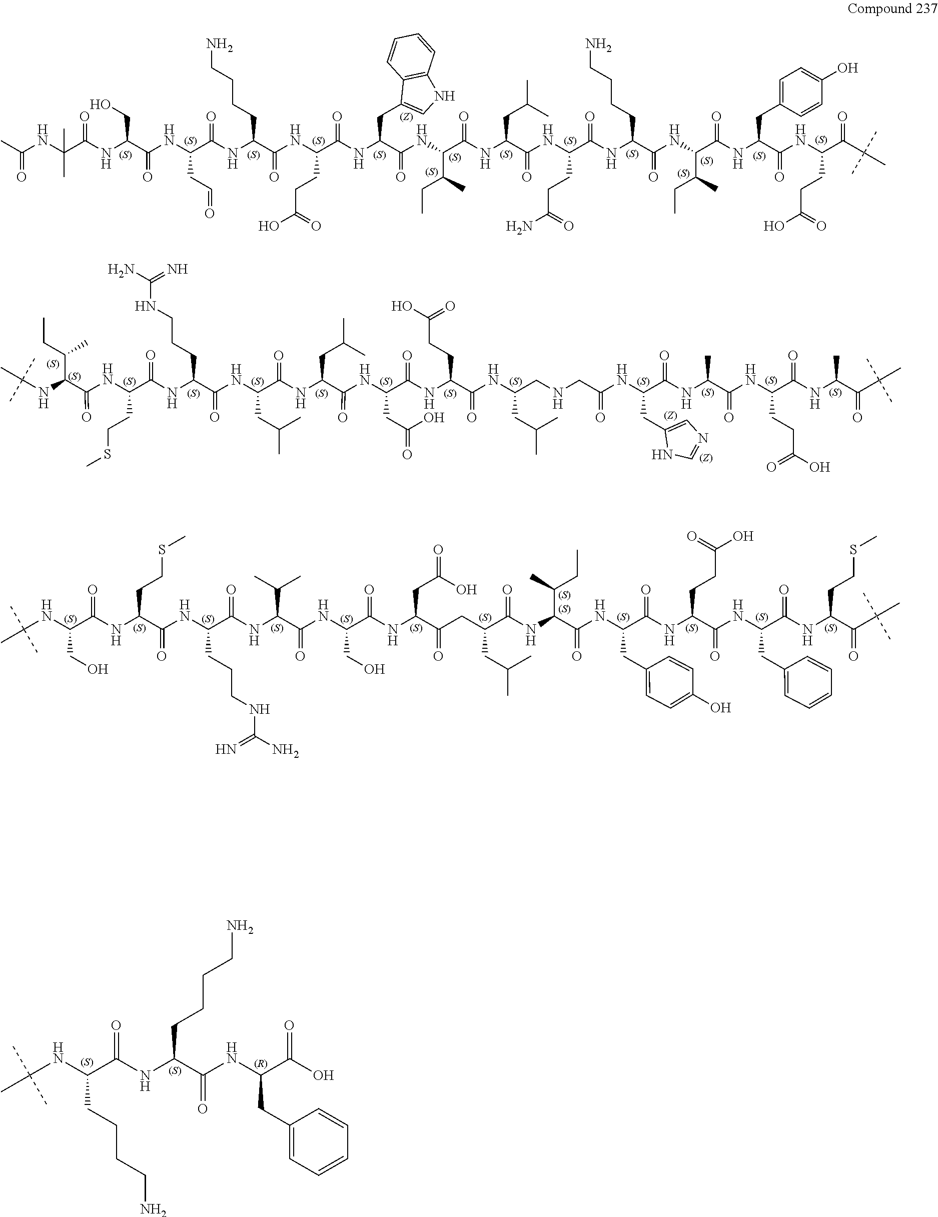

0.20 mmol resin afforded Compound 238 (87.1 mg, 96.6% purity, 6.04% yield) as a white solid. LCMS: RT=1.11 min, MS cal.:4809.56, [M+3H]³⁺=1603.75, [M+4H]⁴⁺=1203.15, [M+5H]⁵⁺=962.61, [M+6H]⁶⁺=802.33, [M+7H]⁷⁺=687.70.

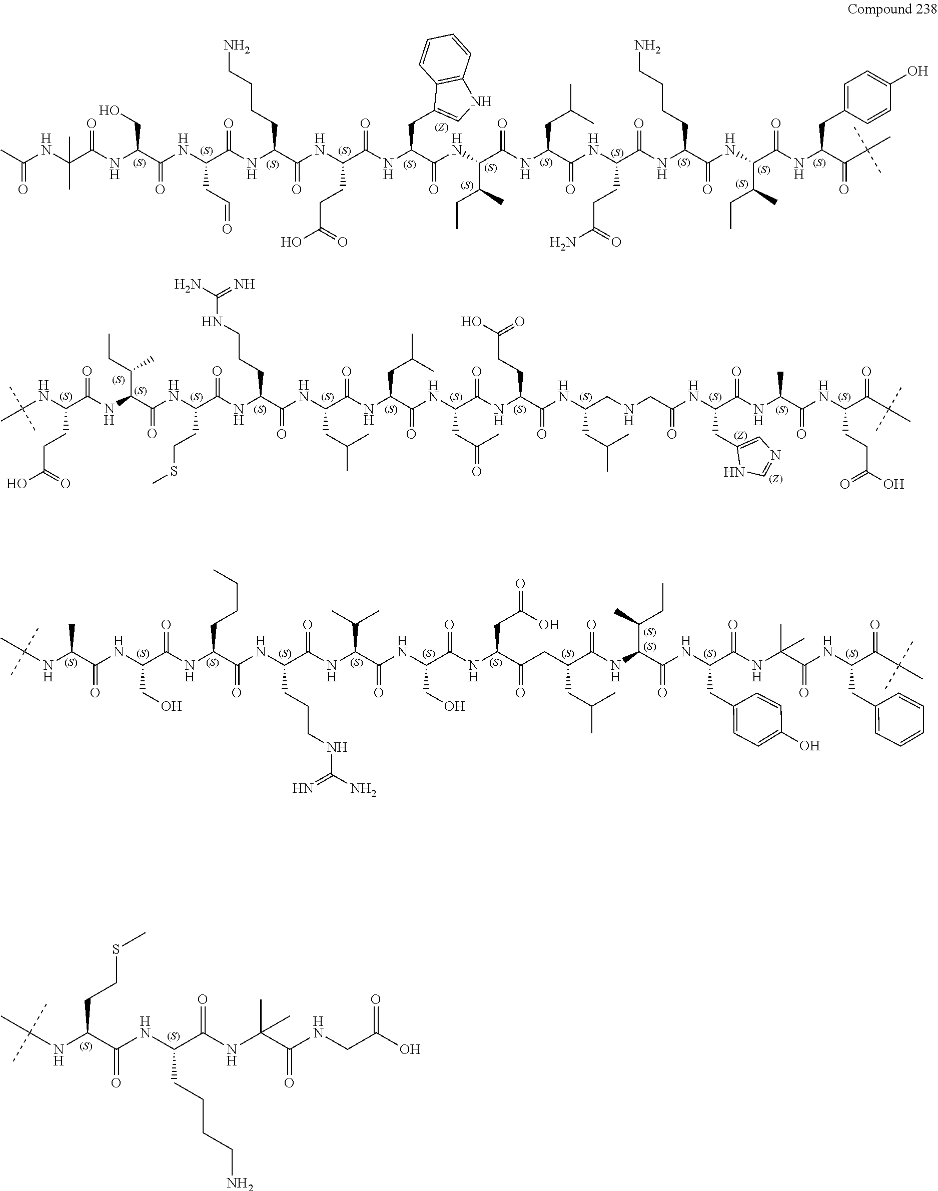

0.20 mmol resin afforded Compound 239 (47.8 mg, 94.0% purity, 4.68% yield) as a white solid. LCMS: RT=1.11 min, MS cal.:4791.52, [M+3H]³⁺=1597.80, [M+4H]⁴⁺=1198.58, [M+5H]⁵⁺=959.12, [M+6H]⁶⁺=799.30, [M+7H]⁷⁺=685.18.

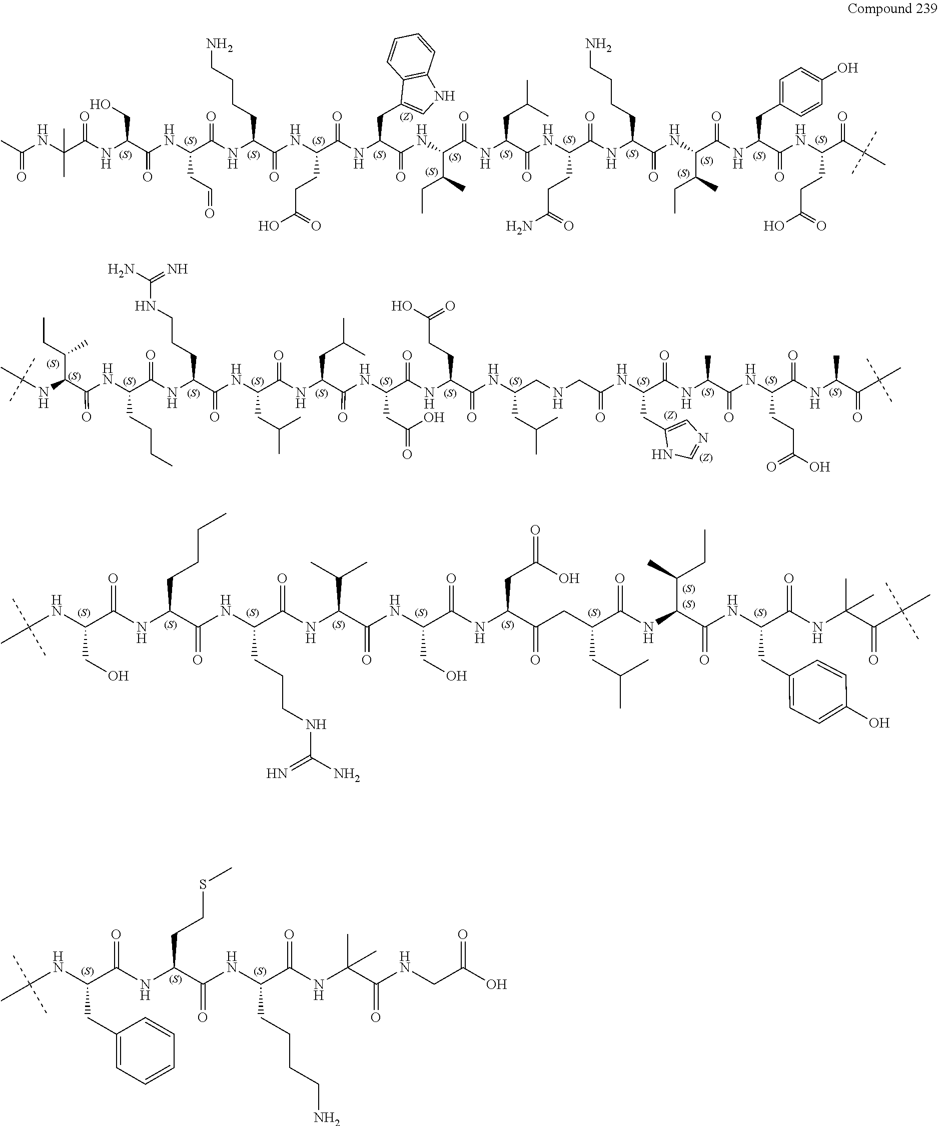

0.20 mmol resin afforded Compound 240 (21.9 mg, 92.5% purity, 2.10% yield) as a white solid. LCMS: RT=1.00 min, MS cal.:4822.58, [M+3H]³⁺=1608.20, [M+4H]⁴⁺+=1206.50, [M+5H]⁵⁺=965.29, [M+6H]⁶⁺=804.51.

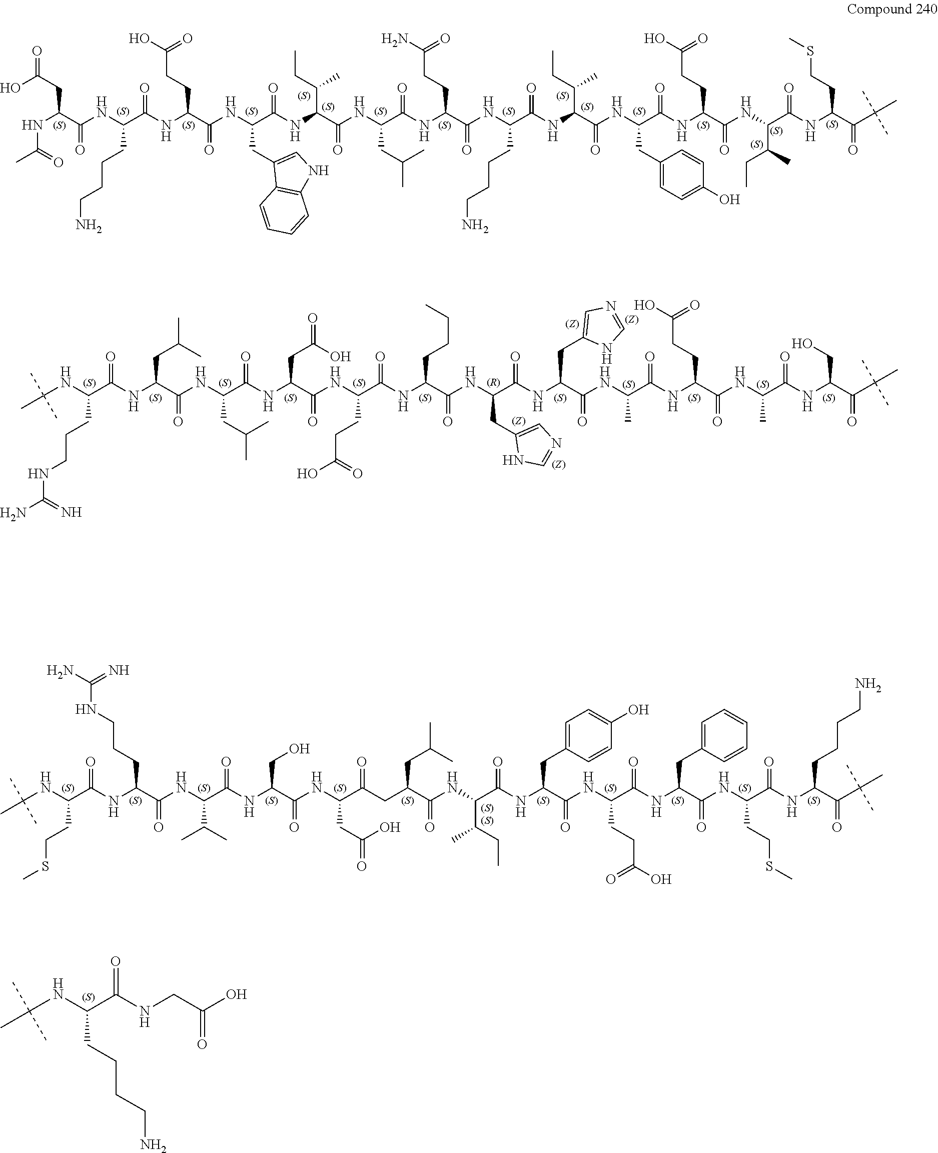

0.20 mmol resin afforded Compound 255 (28.9 mg, 96.6% purity, 2.10% yield) as a white solid. TOF: RT=1.40 min, MS cal.:7699.77, [M+4H]⁴⁺=1925.50, [M+5H]⁵⁺=1540.60, [M+6H]⁶⁺=1284.00.

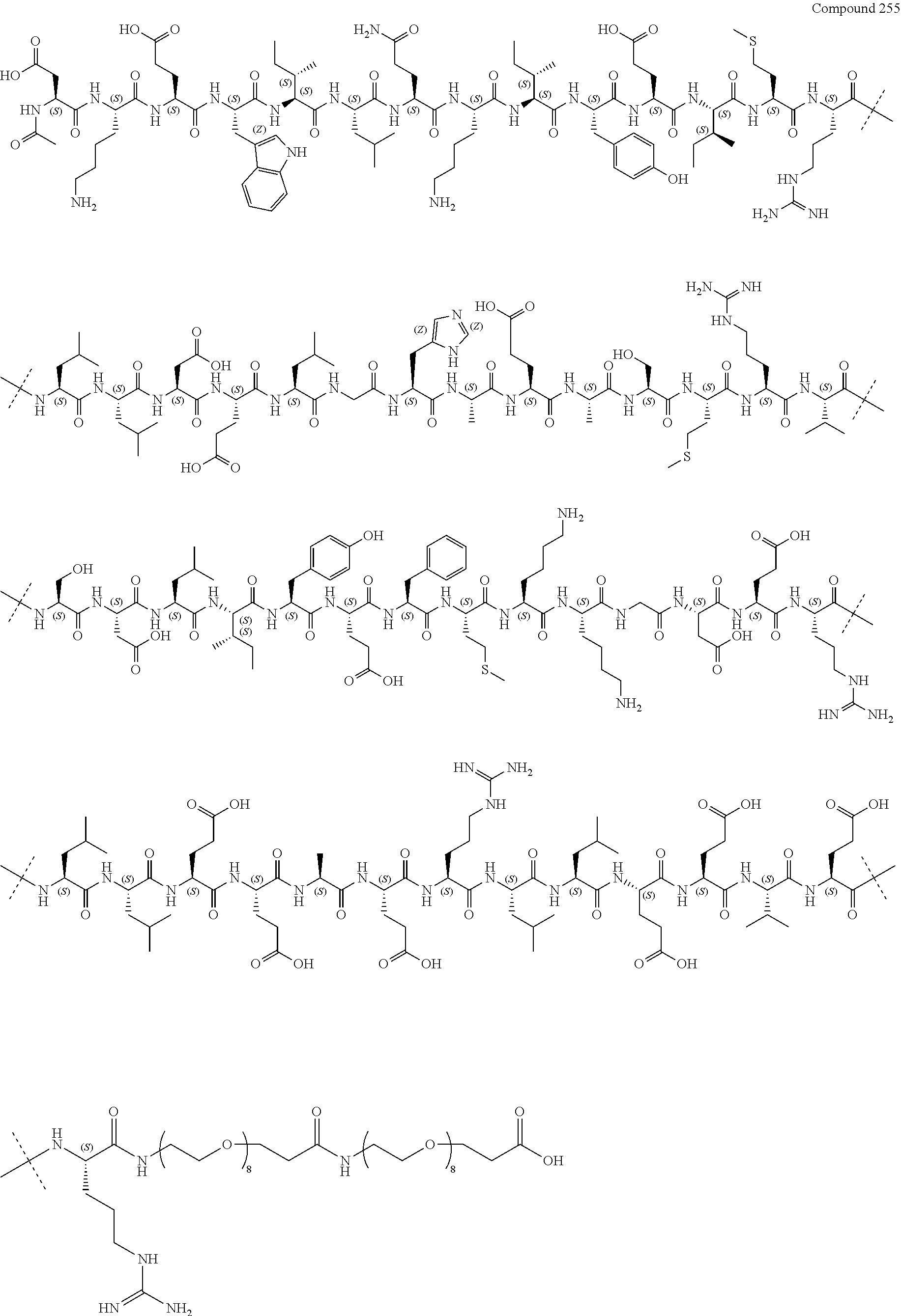

### Example 22. Procedure for Preparation of Compound 241 Via the Click Reaction

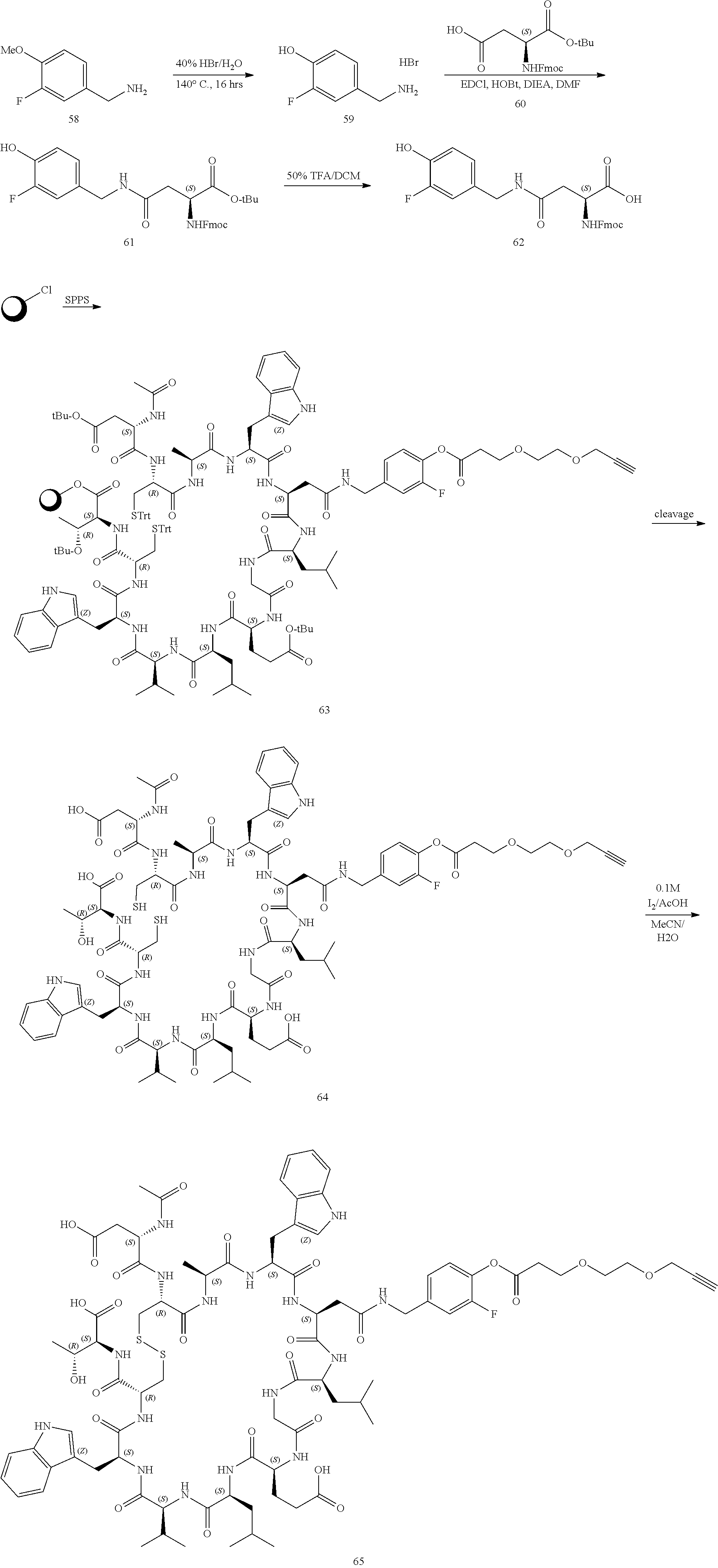

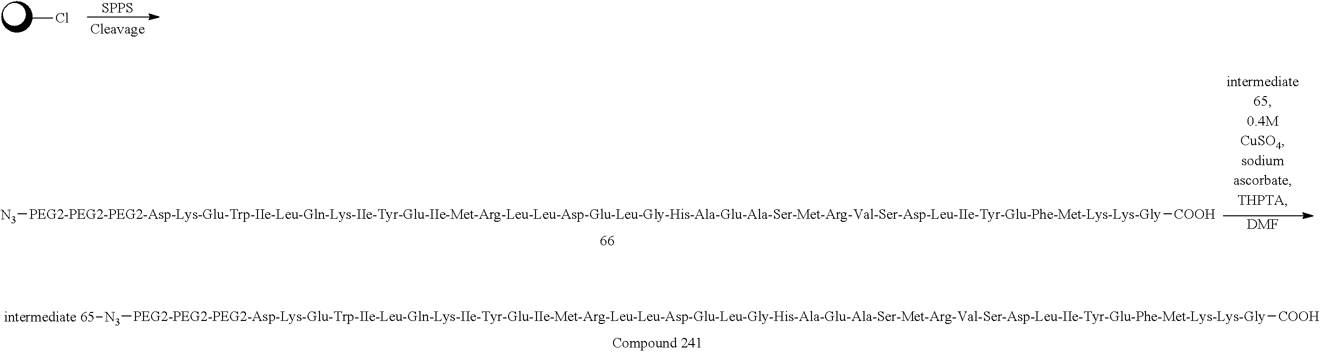

### Preparation of Intermediate 61

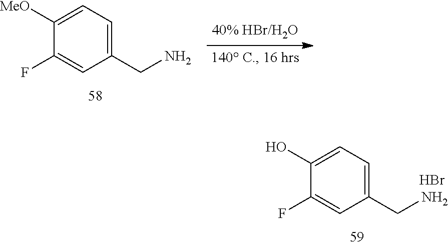 [SMILES: COc1ccc(CN)cc1F ; NCc1ccc(O)c(F)c1 ; Br]

A mixture of intermediate 58 (75.0 g, 64.4 mmol) in HBr/H₂O (40% HBr, 1.00 L in total) was stirred at 140° C. for 16 h. The solvent was removed at 70° C. under reduced pressure. The residue was triturated in MeCN (50 mL) for 10 min. After filtration, the solid was dried under lyophilization to get intermediate 59 (100.0 g, 427.9 mmol, 88.4% yield, HBr salt) as a brown solid. ¹H NMR (400 MHZ, DMSO-d₆) δ ppm 10.03 (s, 1 H), 8.20 (s, 3 H), 7.32 (dd, J=12.17, 1.88 Hz, 1 H), 7.11 (dd, J =8.28, 1.51 Hz, 1 H), 6.95-7.03 (m, 1 H), 3.93 (two doublet, J=10.8 Hz, 2 H).

Preparation of intermediate 61

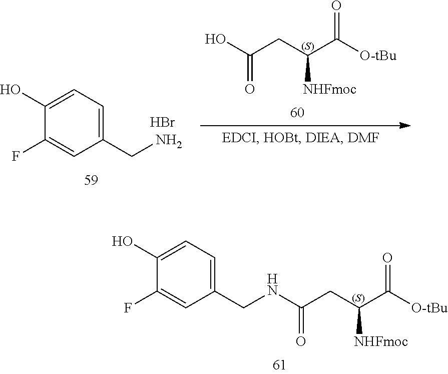

To a mixture of intermediate 59 (60.0 g, 270.2 mmol, 1.00 equiv., HBr), Intermediate 60 (111.1 g, 270.2 mmol, 1.00 equiv.), DIEA (34.9 g, 270.2 mmol, 47.0 mL, 1.00 equiv.), and HOBt (54.7 g, 405.3 mmol, 1.50 equiv.) in DMF (1000 mL) was added EDCI (56.7 g, 297.2 mmol, 1.10 equiv.) at 20° C., the mixture was stirred at 20° C. for 3 h. The mixture was added dropwise to 0.5 M HCl (cold, 10 L) and a white solid was precipitated. After filtration, the solid was dissolved in DCM (2000 mL), washed with 0.5 M HCl (800 mL), H₂O (800 mL), brine (800 mL), dried over anhydrous Na₂SO₄ and concentrated under reduced pressure. The residue was purified by silica gel column (DCM: MeOH=20: 1) to afford intermediate 61 (120.0 g, 90% purity, containing a little DMF, 83.3% yield) as a white solid. H NMR (400 MHz, DMSO-d₆) δ ppm 9.70 (s, 1H), 8.34 (t, J=5.77 Hz, 1H), 7.90 (d, J=7.53 Hz, 2H), 7.71 (d, J=7.53 Hz, 2H), 7.61 (d, J=8.28 Hz, 1H), 7.39-7.47 (m, 2H), 7.29-7.36 (m, 2H), 7.02 (d, J=12.30 Hz, 1H), 6.85-6.92 (m, 2H), 4.20-4.39 (m, 4H), 4.11-4.19 (m, 2H), 2.40-2.70 (m, 2H), 1.36 (s, 9H).

### Preparation of Intermediate 62

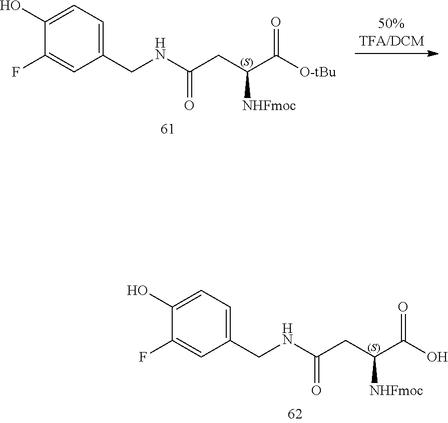

A mixture of intermediate 61 (120.0 g, 224.4 mmol, 1.00 equiv.) in TFA (600 mL) and DCM (600 mL) was stirred at 15° C. for 0.5 h. The solvent was removed under reduced pressure. The residue was purified by silica gel column (DCM: MeOH=10: 1) to intermediate 62 (100 g, 93.5% purity, 93.1% yield) as a white solid. ¹H NMR (400 MHz, DMSO-d₆) δ ppm 9.69 (s, 1H), 8.34 (t, J=5.90 Hz, 1H), 7.90 (d, J=7.28 Hz, 2H), 7.71 (d, J=7.53 Hz, 2H), 7.54 (d, J=6.53 Hz, 1H), 7.42 (t, J=7.40 Hz, 2H), 7.27-7.37 (m, 1H), 7.27-7.37 (m, 1H), 7.02 (d, J=12.05 Hz, 1H), 6.82-6.93 (m, 2H), 4.35-4.43 (m, 1H), 4.20-4.31 (m, 3H), 4.13-4.19 (m, 2H), 2.40-2.70 (m, 2H).

### Preparation of Intermediate 64

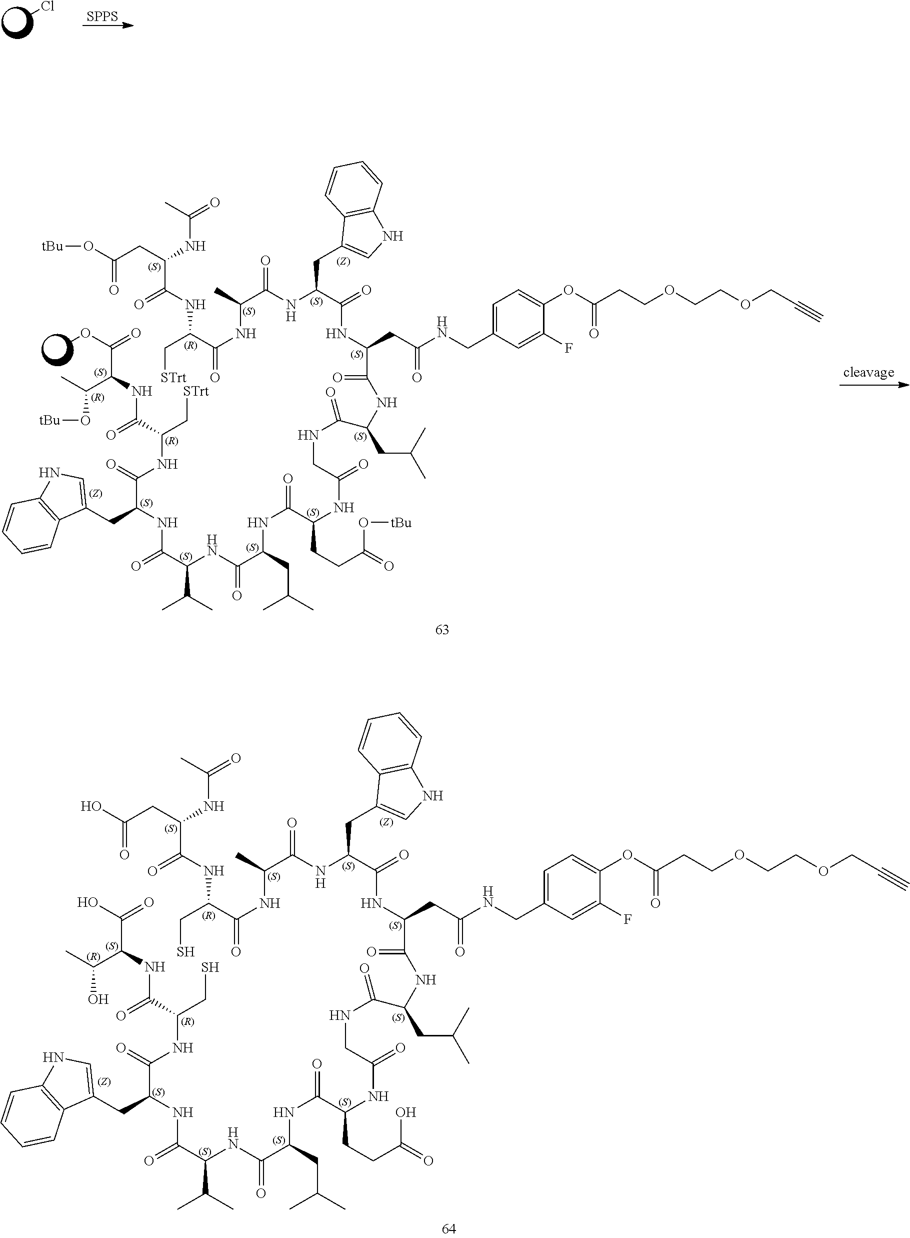

The peptide portion of intermediate 63 was synthesized using standard Fmoc chemistry (CTC resin). Resin preparation: To the vessel containing CTC Resin (20.0 mmol, 20.0 g, 1.00 mmol/g) and Fmoc-Thr(tBu)-OH (7.93 g, 20.0 mmol, 1.00 equiv.) in DCM (300 mL) was added DIEA (4.00 equiv.) dropwise, and mixed for 2 hrs with N₂ bubbling at 20° C. Then MeOH (20.0 mL) was added and bubbled with N₂ for another 30 mins. The resin was washed with DMF (600 mL)*5, followed by adding 20% piperidine in DMF (60 mL) and bubbled with N₂ for 30 mins at 15° C. for Fmoc de-protection. The mixture was filtered and the resin was washed with DMF (600 mL)*5 before proceeding to next step.
1) Coupling: A solution of Fmoc-Cys(Trt)-OH (35.0 g, 3.00 equiv.), HBTU (21.6 g, 2.85 equiv.) in DMF (300 mL) was added to the resin with N₂ bubbling. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (600 mL)*5.
2) De-protection: 20% piperidine in DMF (600 mL) was added to the resin and the mixture was bubbled with N₂ for 30 mins at 20° C. The De-protection reaction was monitored by ninhydrin test, if it showed blue or brownish red, the reaction was completed. The resin was then washed with DMF (600 mL)*5.
3) Steps 1 and 2 were repeated for amino acids: (2-8 in table 17).
4) Coupling: A solution of intermediate 62 (19.1 g, 2.00 equiv.), DIC (5.06 g, 2.00 equiv.), and HOBt (5.46 g, 2.00 equiv.) in DMF (300 mL) was added to the resin with N₂ bubbling for 60 mins at 20° C. The coupling reaction was monitored by ninhydrin test, if it showed colorless, the coupling was completed. The resin was then washed with DMF (600 mL)*5.
4) Step 2 for Fmoc de-protection.
5) Steps 4 and 5 were repeated for amino acids (10-13 in table 17).
6) Acetylation: A solution of 10% Ac₂O/5% NMM/85% DMF (600 mL) was added to resin and the mixture was bubbled with N₂ for 20 mins. The acetylation reaction was monitored by ninhydrin test, if it showed colorless, the coupling was completed. The resin was then washed with DMF (600 mL) *5.
3% hydrazine in DMF (600 mL) was added to resin with N₂ bubbling for 15 min to liberate hydroxyl group on Intermediate 62, the resin was then washed with DMF (600 mL)*5.
7) Coupling for the last residue: A solution of 3-(2-(prop-2-yn-1-yloxy)ethoxy)propanoic acid (10.2 g, 3.00 equiv.), DIC (7.53 g, 3.00 equiv.), HOBt (8.19 g, 3.00 equiv.), and DMAP (7.33 g, 3.00 equiv.) was added to the resin, and the mixture was bubbled with N₂ for 24 hrs. The coupling reaction was monitored by LCMS after a mini-cleavage, almost 50% was desired MS. The resin was then washed with DMF (600 mL)*5, MeOH (600 mL)*5, and then dried under vacuum to afford peptide resin (intermediate 63, 60.3 g).

TABLE 17
The list of amino acids and the corresponding reagents used on SPPS.

| # | Materials | Coupling reagents |
|---|---|---|
| 1 | Fmoc-Thr(tBu)-OH (1.00 equiv.) | DIEA (4.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 |
| | | equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Intermediate 62 (2.00 equiv.) | DIC (2.00 equiv.) and HOBt (2.00 equiv.) |
| 10 | Fmoc-Trp-OH (3.00 equiv.) | DIC (3.00 equiv.) and HOBt (3.00 equiv.) |
| 11 | Fmoc-Ala-OH (3.00 equiv.) | DIC (3.00 equiv.) and HOBt (3.00 equiv.) |
| 12 | Fmoc-Cys(Trt)-OH (3.00 equiv.) | DIC (3.00 equiv.) and HOBt (3.00 equiv.) |
| 13 | Fmoc-Asp(OtBu)-OH (3.00 equiv.) | DIC (3.00 equiv.) and HOBt (3.00 equiv.) |
| 14 | Acetylation | Ac₂O/NMM/DMF (10/5/85, 600 mL) |
| 15 | 3-(2-(prop-2-yn-1- | DIC (3.00 equiv.), HOBt (3.00 equiv.), DMAP |
| | yloxy)ethoxy)propanoic acid (3.00 | (3.00 equiv.) |
| | equiv.) | |

Peptide Cleavage and Purification: Cleavage solution (TFA/Tis/H₂O, 95/2.5/2.5, v/v/v, 600 L) was added to the flask containing the side chain protected peptide resin (intermediate 63, 60.3 g) at room temperature and stirred for 1 h. The filtrate was collected. The peptide was precipitated with cold isopropyl ether (6.0 L). After filtration, the precipitate was washed with cold isopropyl ether (0.5 L)*2, and the solid was dried under vacuum for 2 h. Intermediate 64 (28.5 g, crude) was obtained as a white solid.

### Preparation of Intermediate 65

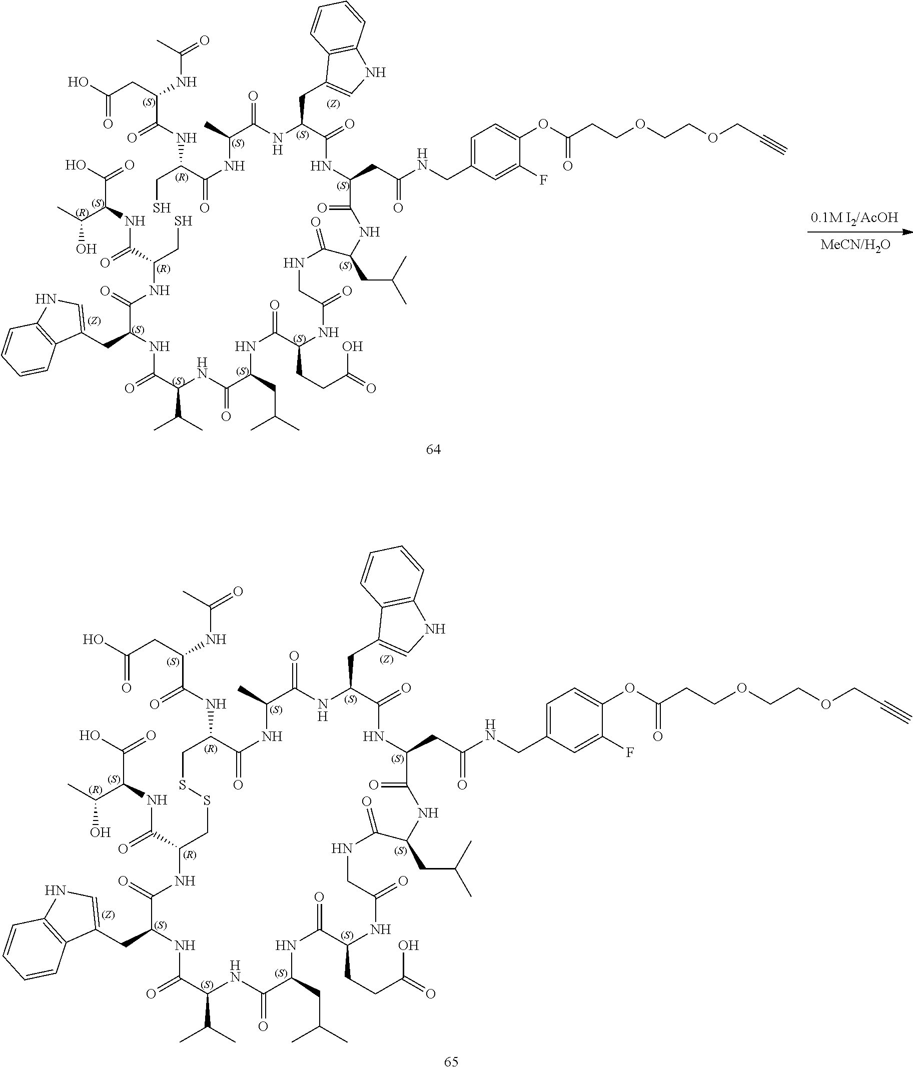

To a mixture of intermediate 64 (9.5 g, crude) in MeCN/H₂O (1/1, v/v, 5.0 L) was added 0.1 M I₂/HOAc dropwise until a yellow color persisted, then the mixture was stirred at 20° C. for 5 mins. The mixture was quenched with 0.1 M Na₂S₂O₃ dropwise until the yellow color disappeared. After filtration, the solution was purified by prep-HPLC (acidic condition, TFA) directly, and lyophilized to afford intermediate 65 (654.59 g, 82.2% purity, 3 batches, 10.3% total yield) as a white solid. LCMS: RT=1.03 min, MS cal.: 1826.73, mass observed: [M+H]⁺=1827.81, [M/2+H]⁺=914.29

### Preparation of Intermediate 66

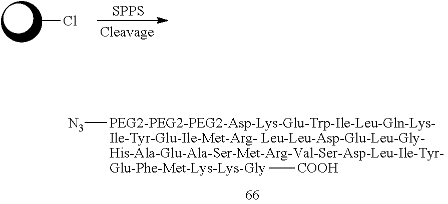

Intermediate 66 (SEQ ID NO:105) was synthesized using standard Fmoc chemistry, and SPPS was identical to that for Compound 203, which was performed by following the procedure mentioned in Example 20.

0.20 mmol resin afforded intermediate 66 obtains (36.1 mg, 90.0% purity, 3.12% yield) as a white solid. LCMS: RT=1.16 min, MS cal.: 5204.00, [M+3H]³⁺=1735.34, [M+4H]⁴⁺+=1302.03, [M+5H]⁵⁺=1042.30.

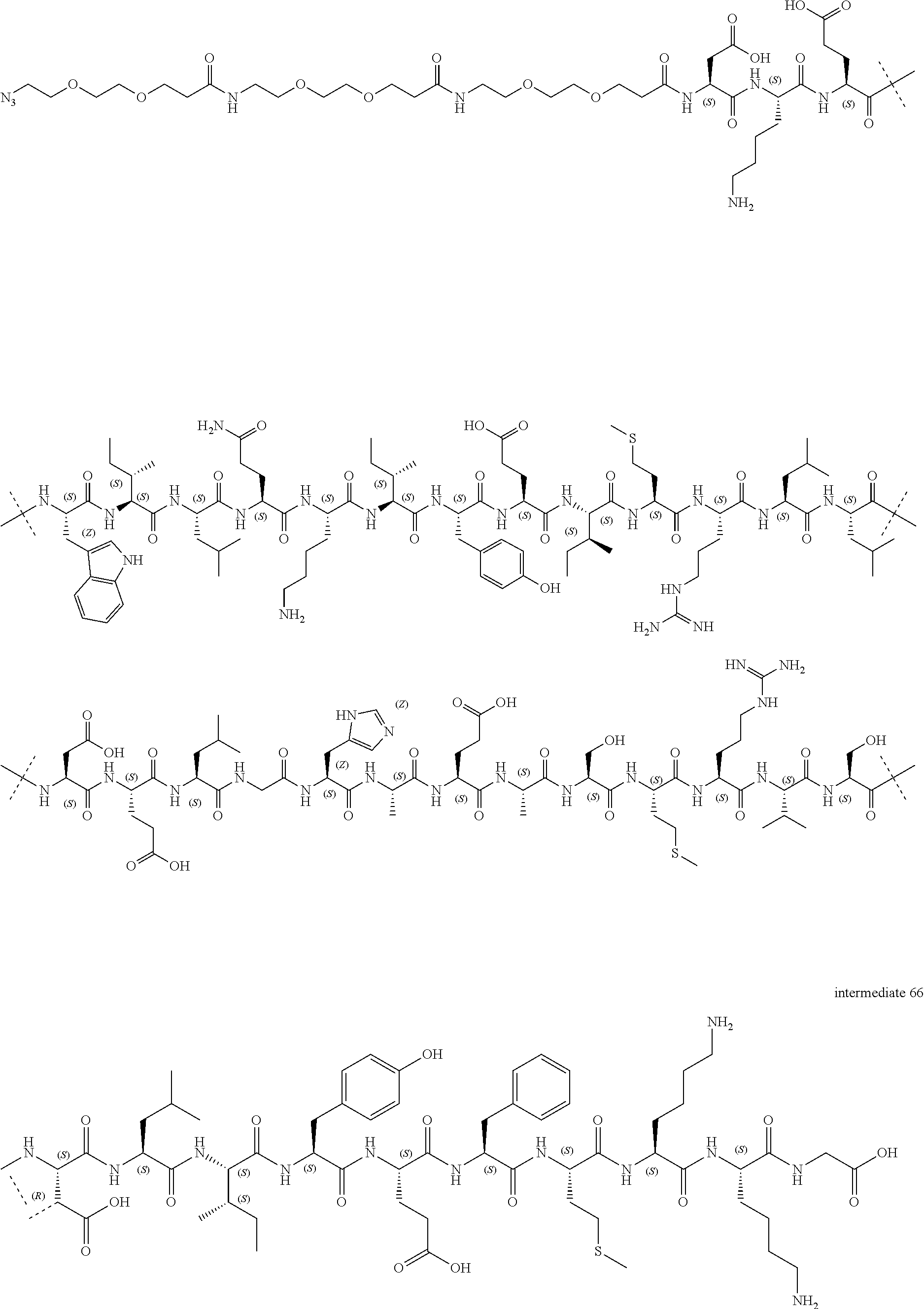

### Preparation of Compound 241

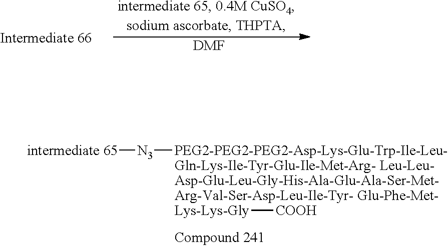

A solution of intermediate 66 (36.1 mg, 1.00 equiv.) and intermediate 65 (12.7 mg, 1.00 equiv.) in DMF (0.5 mL) was cooled to 0° C. under N₂ atmosphere. Then the solution was added a fresh mixture of CuSO₄ (0.4 M, 17.31 uL 1.00 equiv.), THPTA (3.07 mg, 1.00 equiv.) and sodium ascorbate (5.49 mg, 4.00 equiv.) in H₂O (0.10 mL) at 0° C. under N₂ atmosphere. Then the mixture was stirred at 20° C. for 0.5 h under N₂ atmosphere. The mixture was purified by prep-HPLC (A: 0.075% TFA/H₂O, B: MeCN), followed by lyophilization to afford Compound 965 (5.5 mg, 90.2% purity, 0.35% yield) as a white solid. LCMS: RT=1.10 min, MS cal.: 7032.01, [M+4H]⁴⁺+=1758.75, [M+5H]₅+=1407.54, [M+6H]⁶⁺=1173.10.

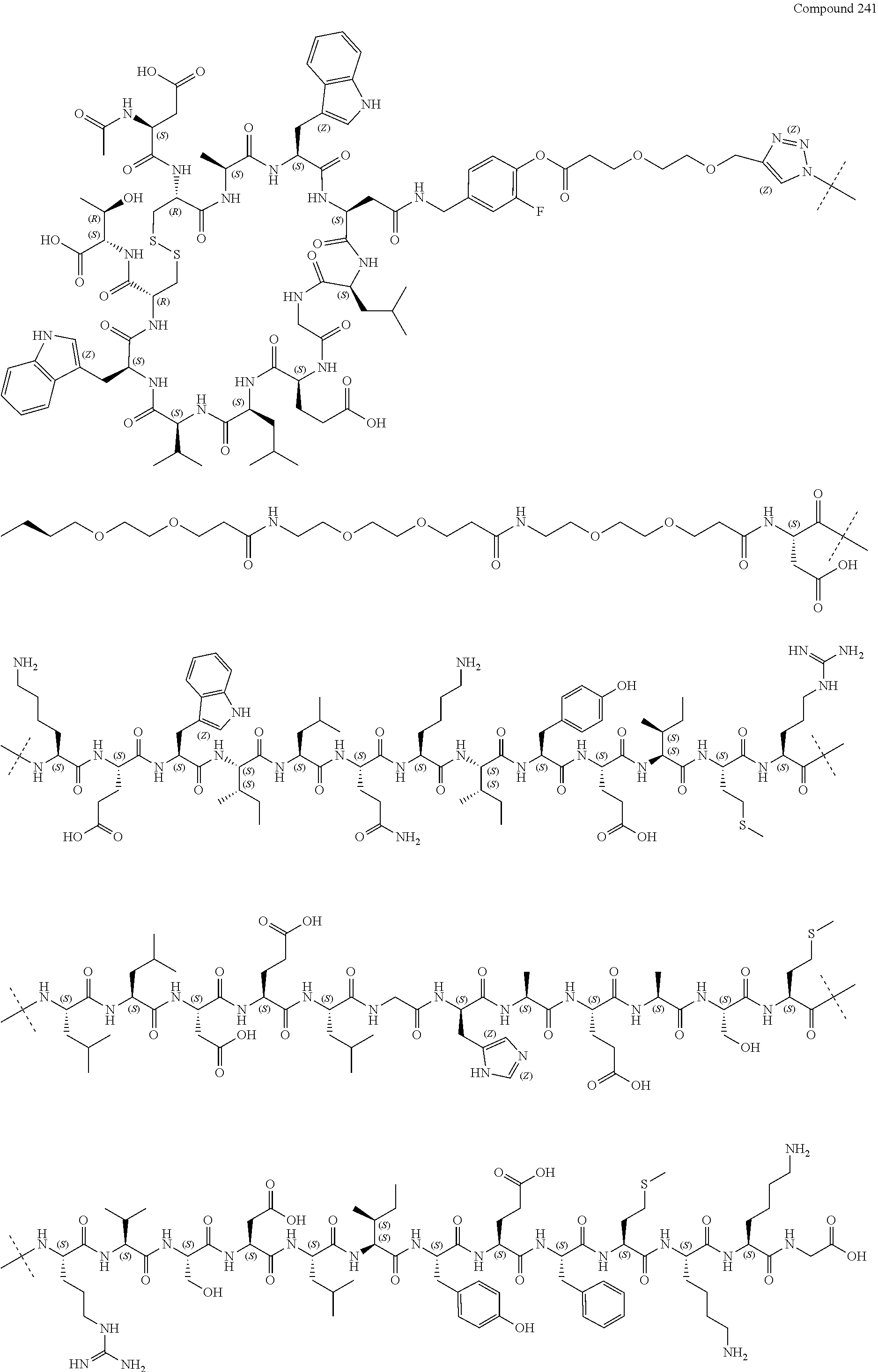

Compounds 242 and 243 were synthesized using the same using the same method as Compound 241. 0.20 mmol resin afforded Compound 242 (2.9 mg, 95.1% purity, 0.14% yield) as a white solid. LCMS: RT=1.10 min, MS cal.: 6517.24, [M+4H]⁴⁺+=1630.08 [M+5H]⁵⁺=1304.44, [M+6H]⁶⁺=1087.12, [M+8H]⁸⁺=815.85, [M+9H]⁹⁺=725.16.

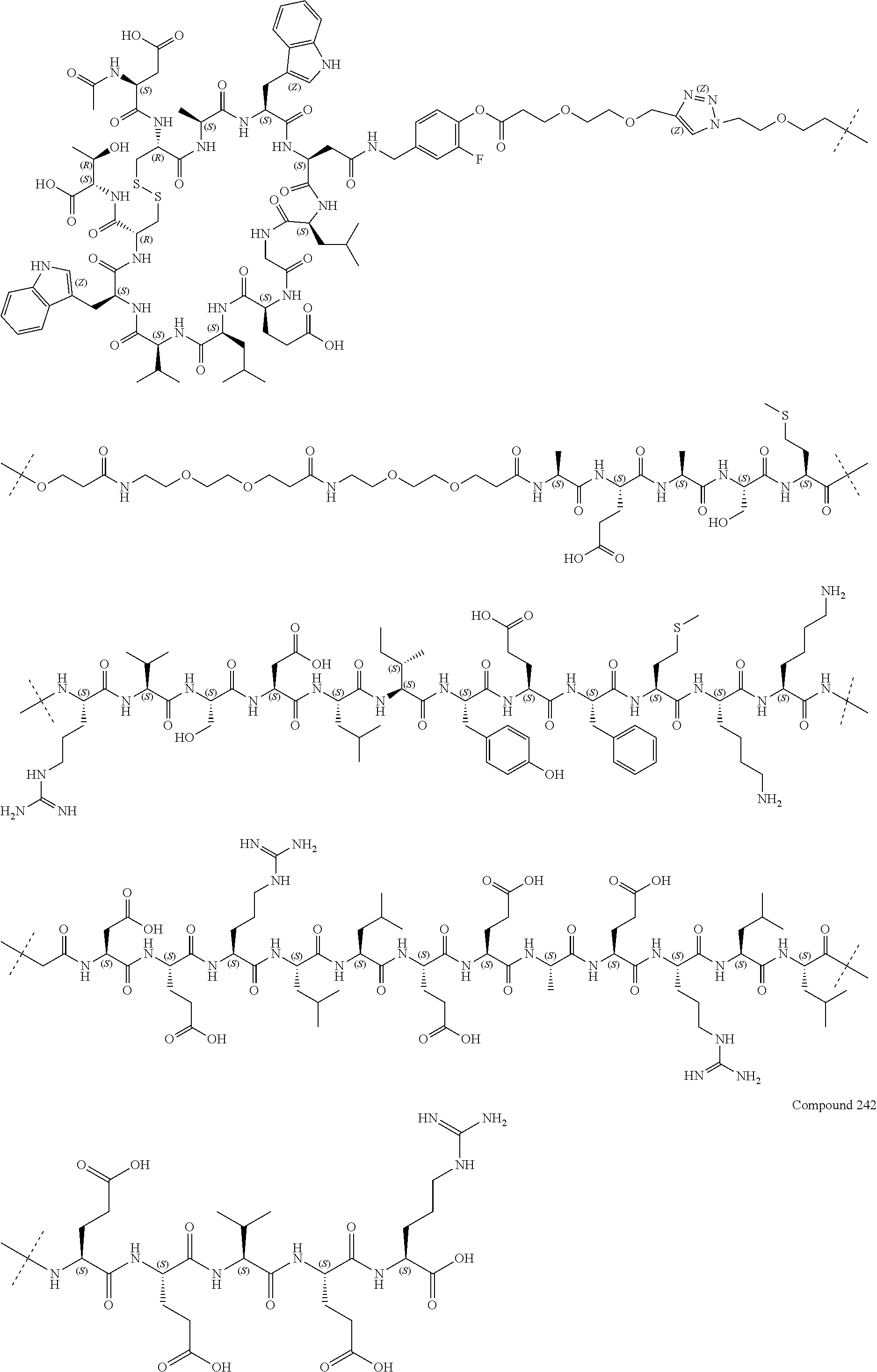

0.20 mmol resin afforded Compound 243 (7.9 mg, 82.4% purity, 0.41% yield) as a white solid. LCMS: RT=1.10 min, MS cal.: 7824.76, [M+6H]⁶⁺=1305.04, [M+7H]⁷⁺=1118.78, [M+8H]⁸⁺978.92, [M+10H]¹⁰⁺=783.37.

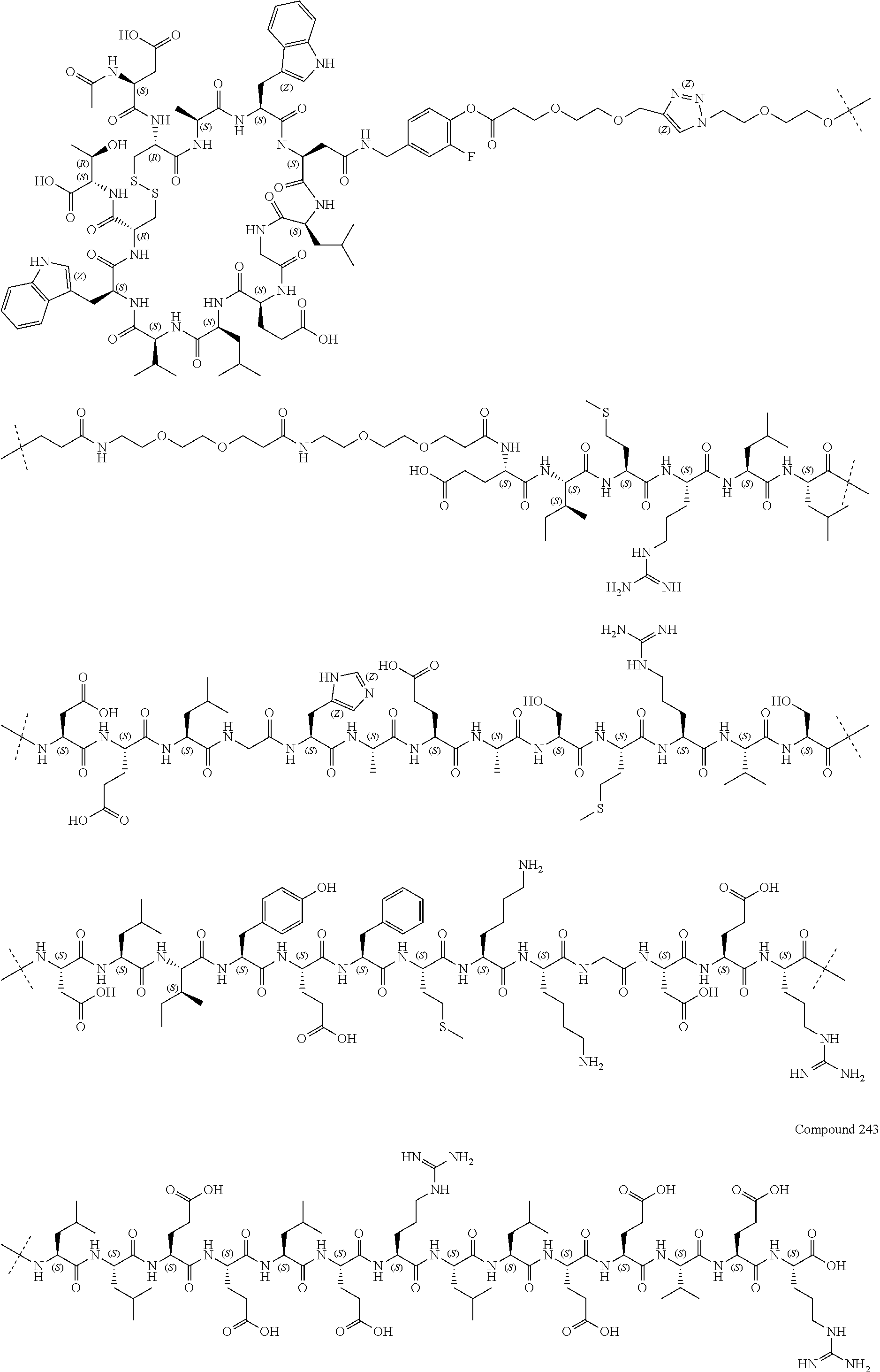

### Example 23. Procedure for Preparation of Compound 244 (Linear Peptide+Diacid+Cyclic Peptide)

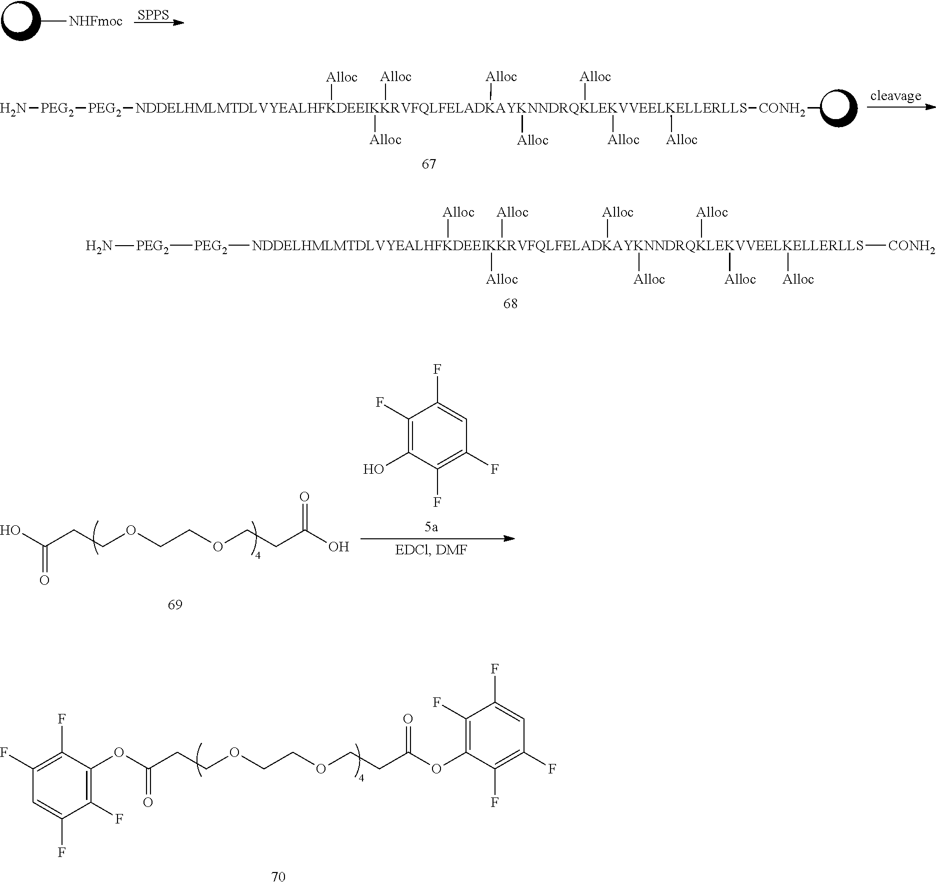

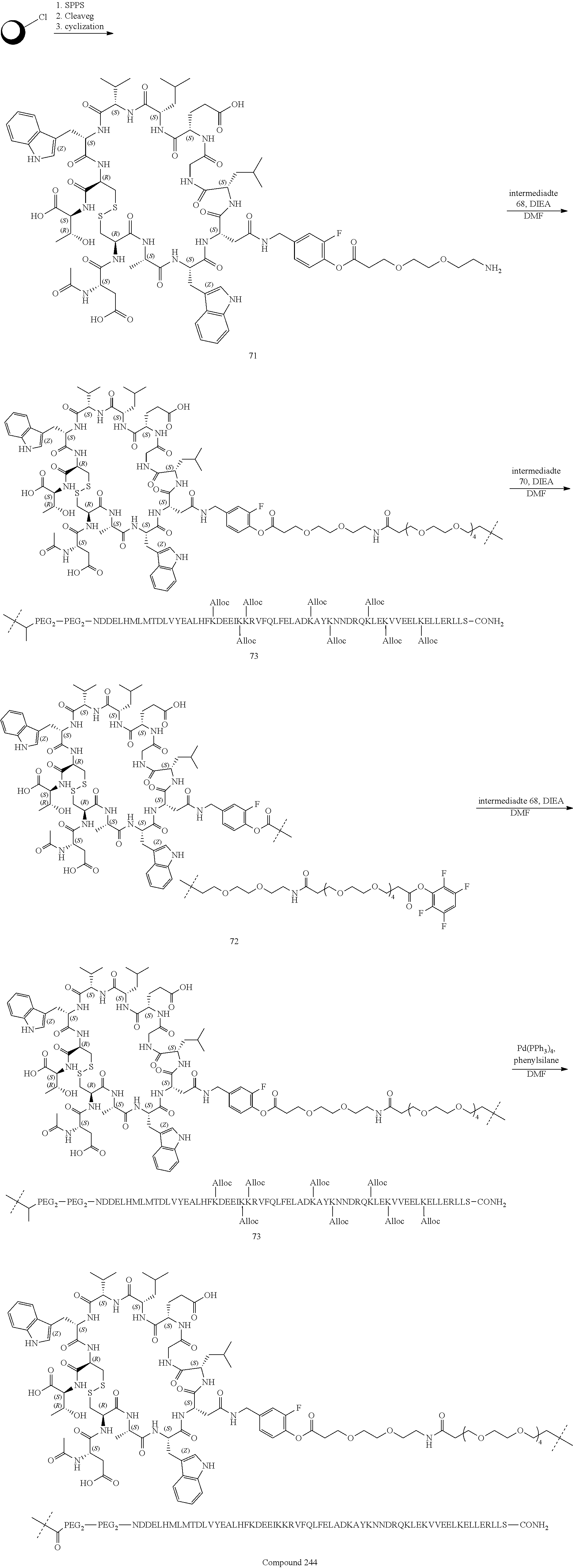

Preparation of Intermediate 68 (resin-bound peptide)

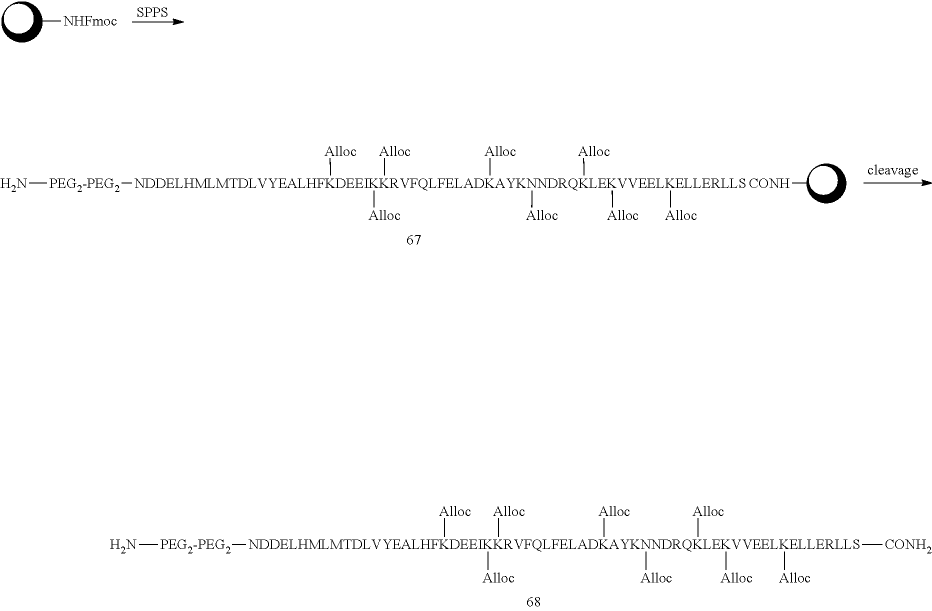

Peptide was synthesized using standard Fmoc chemistry (Rink amide resin). Resin preparation: To the vessel containing Rink Amide AM resin (1.56 g, 0.50 mmol, 0.32 mmol/g) and DMF (20 mL) was bubbled with N₂ for 2 h at 20° C. Then 20% piperidine in DMF (40 mL) was added and the mixture was bubbled with N₂ for 30 min at 20° C. The mixture was filtered and washed with DMF (20 mL)*5 before proceeding to next step.

Coupling: A solution of Fmoc-Ser(tBU)-OH (575.1 mg, 1.50 mmol, 3.00 equiv.), HBTU (541.5 mg, 5.70 mmol, 3.00 equiv.) in DMF (40 mL) was added to the resin with N₂ bubbling. Then DIEA (6.00 equiv.) was added to the mixture dropwise and bubbled with N₂ for 30 min at 20° C. The coupling reaction was monitored by ninhydrin test. The resin was then washed with DMF (80 mL)*5.

Deprotection: 20% piperidine in DMF (80 mL) was added to the resin and the mixture was bubbled with N₂ for 30 min at 20° C. The deprotection reaction was monitored by ninhydrin test. The resin was then washed with DMF (80 mL)*5.

The coupling and deprotection steps were repeated for the following amino acids elongation: Number #2-65, Table 18.

After all the steps were completed, the resin was washed with DMF (80 mL)*5, MeOH (80 mL)*5, then dried under reduced pressure to afford resin-bound peptide intermediate 67 (SEQ ID NO:106) (Rink AM resin, 5.40 g, 0.50 mmol).

TABLE 18
The list of amino acids and the corresponding reagents used on SPPS.

| # | Materials | Coupling reagents |
|---|---|---|
| 1 | Fmoc-Ser(tBu)-OH (1.00 equiv.) | HATU (2.85 equiv.) and DIEA (6.00 equiv.) |
| 2 | Fmoc-Leu-OH (3.00 equiv.) | HATU (2.85 equiv.) and DIEA (6.00 equiv.) |
| 3 | Fmoc-Leu-OH (3.00 equiv.) | HATU (2.85 equiv.) and DIEA (6.00 equiv.) |
| 4 | Fmoc-Arg(Pbf)-OH (3.00 equiv.) | HATU (2.85 equiv.) and DIEA (6.00 equiv.) |
| 5 | Fmoc-Glu(OtBu)-OH (3.00 equiv.) | HATU (2.85 equiv.) and DIEA (6.00 equiv.) |
| 6 | Fmoc-Leu-OH (3.00 equiv.) | HATU (2.85 equiv.) and DIEA (6.00 equiv.) |
| 7 | Fmoc-Leu-OH (3.00 equiv.) | HATU (2.85 equiv.) and DIEA (6.00 equiv.) |
| 8 | Fmoc-Glu(OtBu)-OH (3.00 equiv.) | HATU (2.85 equiv.) and DIEA (6.00 equiv.) |
| 9 | Fmoc-Lys(Alloc)-OH (3.00 equiv.) | HATU (2.85 equiv.) and DIEA (6.00 equiv.) |
| 10 | Fmoc-Leu-OH (3.00 equiv.) | HATU (2.85 equiv.) and DIEA (6.00 equiv.) |
| 11 | Fmoc-Glu(OtBu)-OH (3.00 equiv.) | HATU (2.85 equiv.) and DIEA (6.00 equiv.) |
| 12 | Fmoc-Glu(OtBu)-OH (3.00 equiv.) | HATU (2.85 equiv.) and DIEA (6.00 equiv.) |
| 13 | Fmoc-Val-OH (3.00 equiv.) | HATU (2.85 equiv.) and DIEA (6.00 equiv.) |
| 14 | Fmoc-Val-OH (3.00 equiv.) | HATU (2.85 equiv.) and DIEA (6.00 equiv.) |
| 15 | Fmoc-Lys(Alloc)-OH (3.00 equiv.) | HATU (2.85 equiv.) and DIEA (6.00 equiv.) |
| 16 | Fmoc-Glu(OtBu)-OH (3.00 equiv.) | HATU (2.85 equiv.) and DIEA (6.00 equiv.) |
| 17 | Fmoc-Leu-OH (3.00 equiv.) | HATU (2.85 equiv.) and DIEA (6.00 equiv.) |
| 18 | Fmoc-Lys(Alloc)-OH (3.00 equiv.) | HATU (2.85 equiv.) and DIEA (6.00 equiv.) |
| 19 | Fmoc-Gln(Trt)-OH (3.00 equiv.) | HATU (2.85 equiv.) and DIEA (6.00 equiv.) |
| 20 | Fmoc-Arg(Pbf)-OH (3.00 equiv.) | HATU (2.85 equiv.) and DIEA (6.00 equiv.) |
| 21 | Fmoc-Asp(OtBu)-OH (3.00 equiv.) | HATU (2.85 equiv.) and DIEA (6.00 equiv.) |
| 22 | Fmoc-Asn(Trt)-OH (3.00 equiv.) | HATU (2.85 equiv.) and DIEA (6.00 equiv.) |
| 23 | Fmoc-Asn(Trt)-OH (3.00 equiv.) | HATU (2.85 equiv.) and DIEA (6.00 equiv.) |
| 24 | Fmoc-Lys(Alloc)-OH (3.00 equiv.) | HATU (2.85 equiv.) and DIEA (6.00 equiv.) |
| 25 | Fmoc-Tyr(tBu)-OH (3.00 equiv.) | HATU (2.85 equiv.) and DIEA (6.00 equiv.) |
| 26 | Fmoc-Ala-OH (3.00 equiv.) | HATU (2.85 equiv.) and DIEA (6.00 equiv.) |
| 27 | Fmoc-Lys(Alloc)-OH (3.00 equiv.) | HATU (2.85 equiv.) and DIEA (6.00 equiv.) |
| 28 | Fmoc-Asp(OtBu)-OH (3.00 equiv.) | HATU (2.85 equiv.) and DIEA (6.00 equiv.) |
| 29 | Fmoc-Ala-OH (3.00 equiv.) | HATU (2.85 equiv.) and DIEA (6.00 equiv.) |
| 30 | Fmoc-Leu-OH (3.00 equiv.) | HATU (2.85 equiv.) and DIEA (6.00 equiv.) |
| 31 | Fmoc-Glu(OtBu)-OH (3.00 equiv.) | HATU (2.85 equiv.) and DIEA (6.00 equiv.) |
| 32 | Fmoc-Phe-OH (3.00 equiv.) | HATU (2.85 equiv.) and DIEA (6.00 equiv.) |
| 33 | Fmoc-Leu-OH (3.00 equiv.) | HATU (2.85 equiv.) and DIEA (6.00 equiv.) |
| 34 | Fmoc-Gln(Trt)-OH (3.00 equiv.) | HATU (2.85 equiv.) and DIEA (6.00 equiv.) |
| 35 | Fmoc-Phe-OH (3.00 equiv.) | HATU (2.85 equiv.) and DIEA (6.00 equiv.) |
| 36 | Fmoc-Val-OH (3.00 equiv.) | HATU (2.85 equiv.) and DIEA (6.00 equiv.) |
| 37 | Fmoc-Arg(Pbf)-OH (3.00 equiv.) | HATU (2.85 equiv.) and DIEA (6.00 equiv.) |
| 38 | Fmoc-Lys(Alloc)-OH (3.00 equiv.) | HATU (2.85 equiv.) and DIEA (6.00 equiv.) |
| 39 | Fmoc-Lys(Alloc)-OH (3.00 equiv.) | HATU (2.85 equiv.) and DIEA (6.00 equiv.) |
| 40 | Fmoc-Ile-OH (3.00 equiv.) | HATU (2.85 equiv.) and DIEA (6.00 equiv.) |
| 41 | Fmoc-Glu(OtBu)-OH (3.00 equiv.) | HATU (2.85 equiv.) and DIEA (6.00 equiv.) |
| 42 | Fmoc-Glu(OtBu)-OH (3.00 equiv.) | HATU (2.85 equiv.) and DIEA (6.00 equiv.) |
| 43 | Fmoc-Asp(OtBu)-OH (3.00 equiv.) | HATU (2.85 equiv.) and DIEA (6.00 equiv.) |
| 44 | Fmoc-Lys(Alloc)-OH (3.00 equiv.) | HATU (2.85 equiv.) and DIEA (6.00 equiv.) |
| 45 | Fmoc-Phe-OH (3.00 equiv.) | HATU (2.85 equiv.) and DIEA (6.00 equiv.) |
| 46 | Fmoc-His(Trt)-OH (3.00 equiv.) | HATU (2.85 equiv.) and DIEA (6.00 equiv.) |
| 47 | Fmoc-Leu-OH (3.00 equiv.) | HATU (2.85 equiv.) and DIEA (6.00 equiv.) |
| 48 | Fmoc-Ala-OH (3.00 equiv.) | HATU (2.85 equiv.) and DIEA (6.00 equiv.) |
| 49 | Fmoc-Glu(OtBu)-OH (3.00 equiv.) | HATU (2.85 equiv.) and DIEA (6.00 equiv.) |
| 50 | Fmoc-Tyr(tBu)-OH (3.00 equiv.) | HATU (2.85 equiv.) and DIEA (6.00 equiv.) |
| 51 | Fmoc-Val-OH (3.00 equiv.) | HATU (2.85 equiv.) and DIEA (6.00 equiv.) |
| 52 | Fmoc-Leu-OH (3.00 equiv.) | HATU (2.85 equiv.) and DIEA (6.00 equiv.) |
| 53 | Fmoc-Asp(OtBu)-OH (3.00 equiv.) | HATU (2.85 equiv.) and DIEA (6.00 equiv.) |
| 54 | Fmoc-Thr(tBu)-OH (3.00 equiv.) | HATU (2.85 equiv.) and DIEA (6.00 equiv.) |
| 55 | Fmoc-Met-OH (3.00 equiv.) | HATU (2.85 equiv.) and DIEA (6.00 equiv.) |
| 56 | Fmoc-Leu-OH (3.00 equiv.) | HATU (2.85 equiv.) and DIEA (6.00 equiv.) |
| 57 | Fmoc-Met-OH (3.00 equiv.) | HATU (2.85 equiv.) and DIEA (6.00 equiv.) |
| 58 | Fmoc-His(Trt)-OH (3.00 equiv.) | HATU (2.85 equiv.) and DIEA (6.00 equiv.) |
| 59 | Fmoc-Leu-OH (3.00 equiv.) | HATU (2.85 equiv.) and DIEA (6.00 equiv.) |
| 60 | Fmoc-Glu(OtBu)-OH (3.00 equiv.) | HATU (2.85 equiv.) and DIEA (6.00 equiv.) |
| 61 | Fmoc-Asp(OtBu)-OH (3.00 equiv.) | HATU (2.85 equiv.) and DIEA (6.00 equiv.) |
| 62 | Fmoc-Asp(OtBu)-OH (3.00 equiv.) | HATU (2.85 equiv.) and DIEA (6.00 equiv.) |
| 63 | Fmoc-Asn(Trt)-OH (3.00 equiv.) | HATU (2.85 equiv.) and DIEA (6.00 equiv.) |
| 64 | Fmoc-PEG2-CH₂CH₂COOH (2.00 equiv.) | HATU (1.90 equiv.) and DIEA (4.00 equiv.) |
| 65 | Fmoc-PEG2-CH₂CH₂COOH (2.00 equiv.) | HATU (1.90 equiv.) and DIEA (4.00 equiv.) |

Peptide cleavage, cyclization and purification. Cleavage solution (TFA/TIS/H₂O, 95/2.5/2.5, v/v/v, 100 mL) was added to the flask containing the side-chain protected resin-bound peptide intermediate 67 (Rink AM resin, 0.5 mmol, 5.40 g) at room temperature and stirred for 2 h.

After filtration, the filtrate was collected.

The peptide was precipitated with cold isopropyl ether (500 mL) and centrifuged (3 min at 3000 rpm).

Isopropyl ether washed two additional times, and the crude peptide was dried under vacuum for 2 h to afford intermediate 68 (SEQ ID NO: 106) (3.05 g, crude) as a white solid.

The crude was purified by prep-HPLC (A: 0.075% TFA/H₂O, B: MeCN), followed by lyophilization to afford intermediate 68 (201.5 mg, 85% purity, 3.9% yield) as a white solid.

### Preparation of Intermediate 70

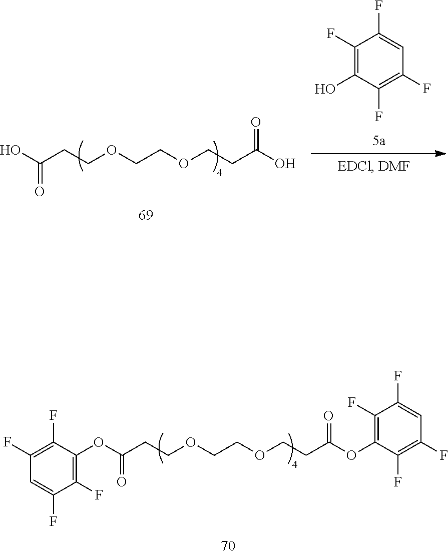

A mixture of intermediate 69 (10 g, 21.25 mmol, 1.00 equiv.), intermediate 5a (21.18 g, 127.52 mmol, 6 equiv.), EDCI (16.30 g, 85.01 mmol, 4 equiv.) in DMF (100 mL) was stirred at 20° C. for 16 h. Then the mixture was purified by prep-HPLC (A: 0.075% TFA/H₂O, B: MeCN) directly to afford intermediate 70 (14.0 g, 17.35 mmol, 95% purity, 81.63% yield) as a yellow oil. LCMS: RT=1.296 min, MS cal.: 766.62, mass observed: [M+H]⁺=767.5, [M+H₂O+H]⁺=785.5, [M+Na]⁺=789.5.

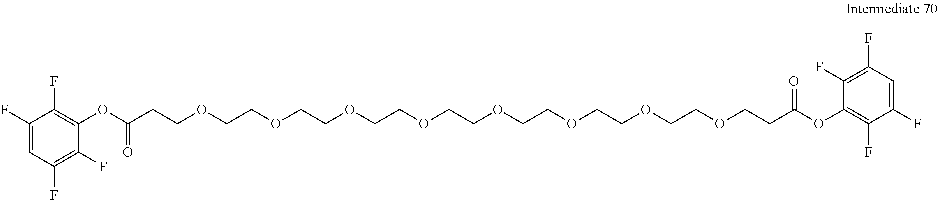 [SMILES: O=C(CCOCCOCCOCCOCCOCCOCCOCCOCCC(=O)Oc1c(F)c(F)cc(F)c1F)Oc1c(F)c(F)cc(F)c1F]

### Preparation of Intermediate 71

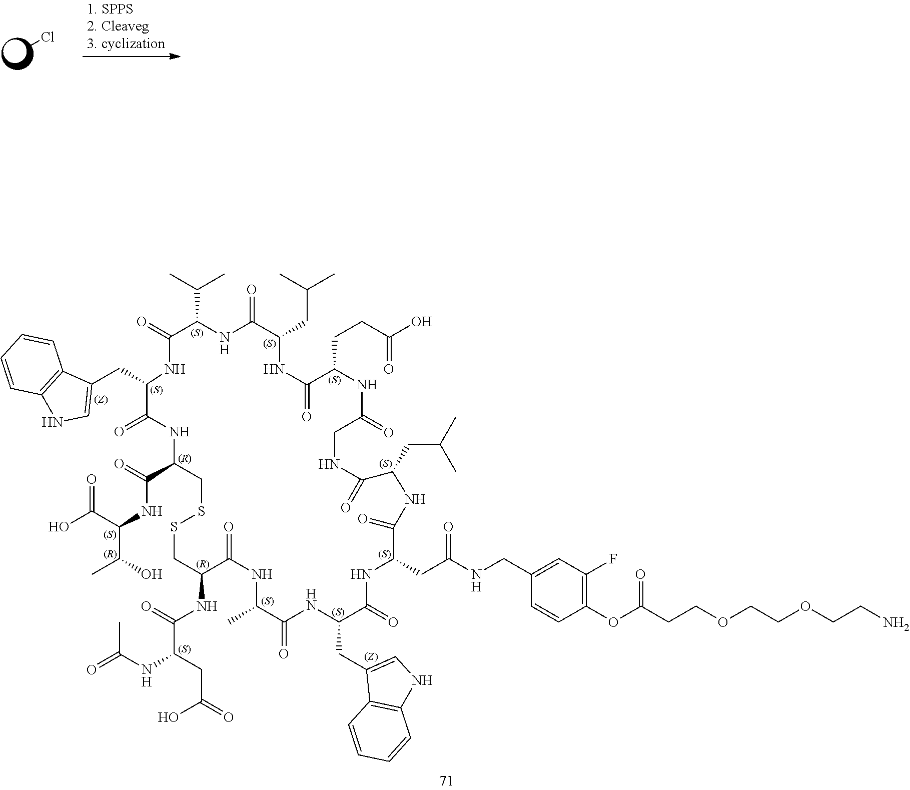

Intermediate 71 was synthesized using the same using the same method as intermediate 65. 100 mmol resin afford intermediate 71 (24.8 g, 93.9% purity, 15.3% yield) as a white solid. LCMS: RT=1.417 min, MS cal.: 1833.02, mass observed: [M+2H]²⁺=917.3.

### Preparation of Intermediate 72

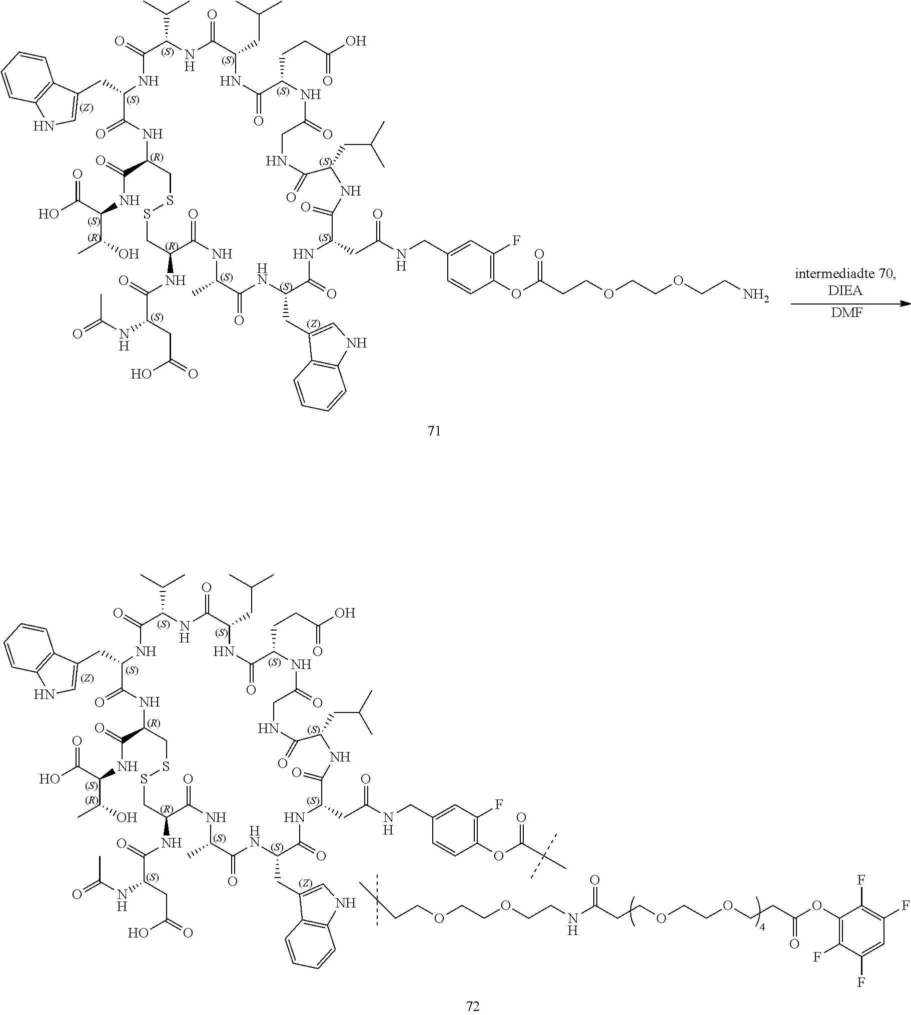

To a mixture of intermediate 70 (1.05 g, 1.36 mmol, 5.00 equiv.) in DMF (5 mL) was added a mixture of intermediate 71 (500.0 mg, 272.77 μmol, 1.00 equiv.) DIEA (246.78 mg, 1.91 mmol, 332.59 uL, 7.00 equiv.) in DMSO (10 mL) dropwise at 0° C. Then the mixture was stirred at 15° C. for 10 mins. The mixture was purified by prep-HPLC (A: 0.075% TFA/H₂O, B: MeCN) directly to afford intermediate 72 (350.0 mg, 143.82 μmol, 52.73% yield) as a white solid. LCMS: RT=1.199 min, MS cal.: 2433.57, mass observed: [M+2H]²⁺=1217.15.

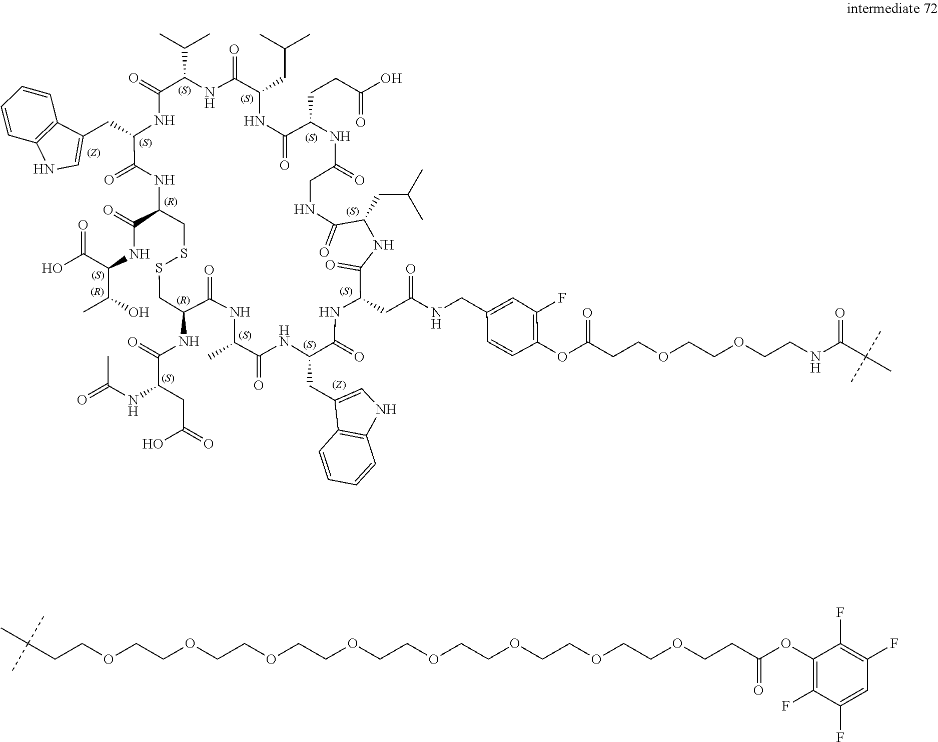

### Preparation of Intermediate 73

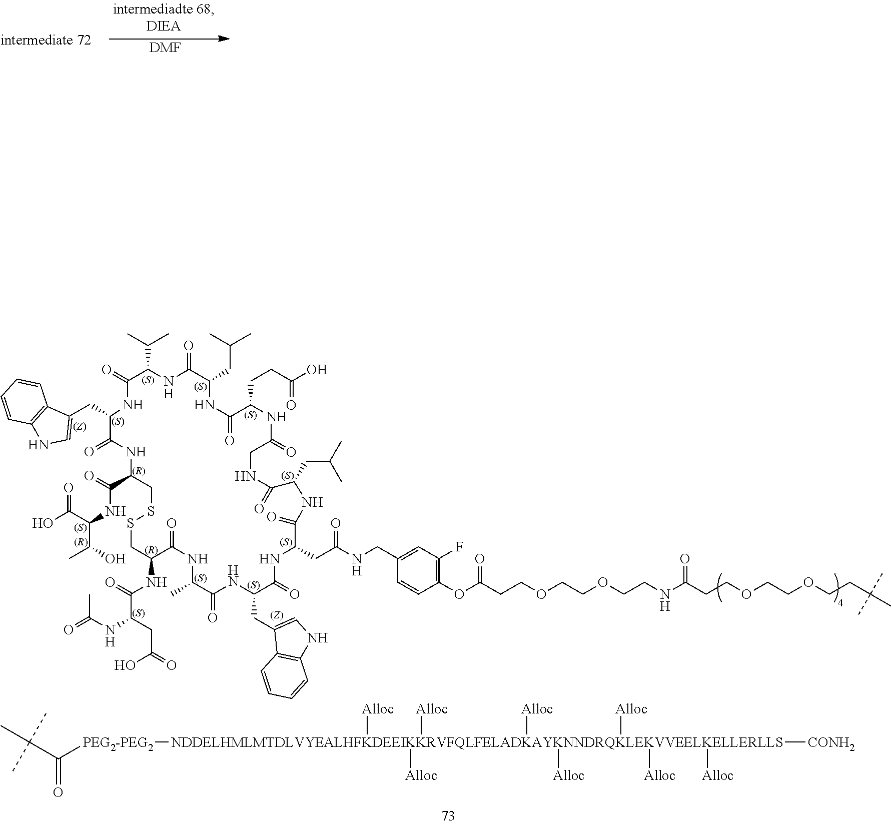

(antibody binding moiety linked to SEQ ID NO:106)

A mixture of intermediate 72 (46.50 mg, 21.44 μmol, 2.00 equiv.) and intermediate 69 (80 mg, 10.72 μmol, 1.00 equiv.), DIEA (8.31 mg, 64.31 μmol, 11.20 uL, 6.00 equiv.) in DMSO (2.00 mL) was stirred at 20° C. for 1 h. The mixture was purified by prep-HPLC (A: 0.075% TFA/H₂O, B: MeCN) directly to afford intermediate 73 (45.0 mg, 90% purity, 39.9% yield) as a white solid. LCMS: RT=1.454 min, MS cal.: 10994.25, mass observed: [M+6H]⁶⁺=1833.45, [M+7H]⁷⁺=1571.40, [M+8H]⁸⁺1375.15, [M+9H]⁹⁺=1222.40.

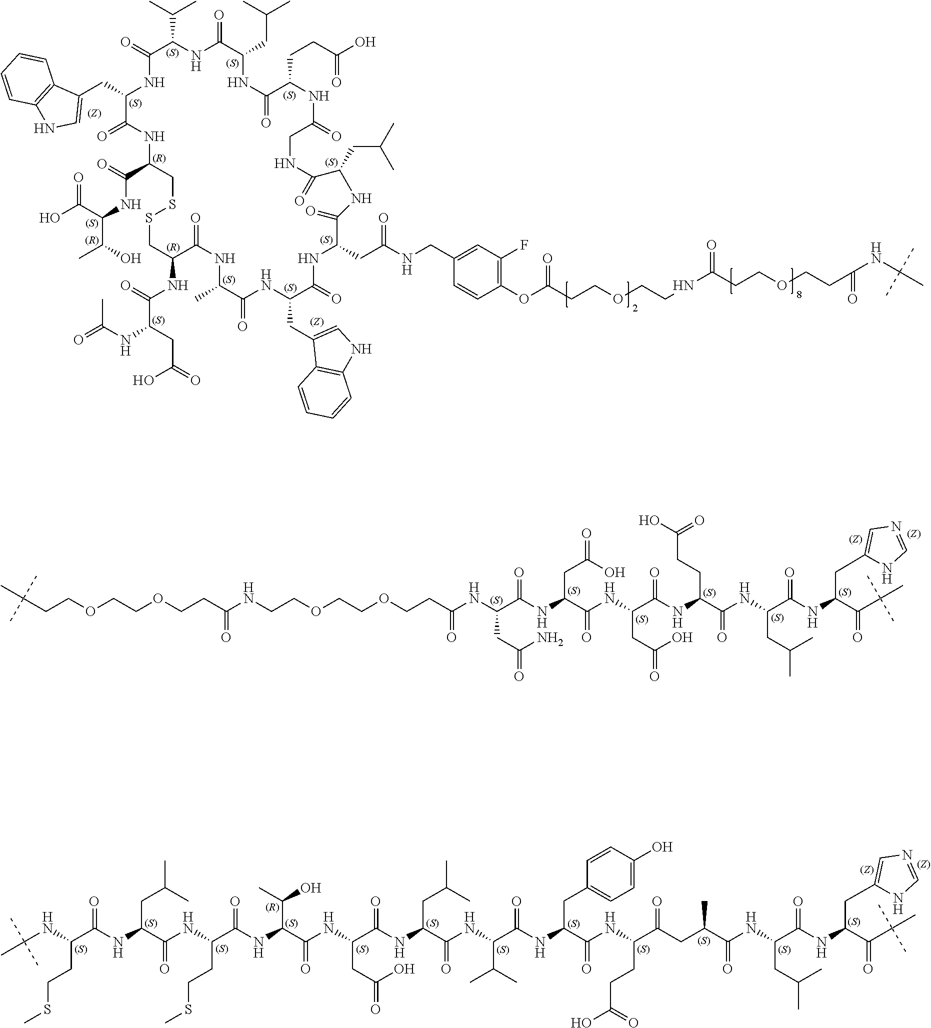

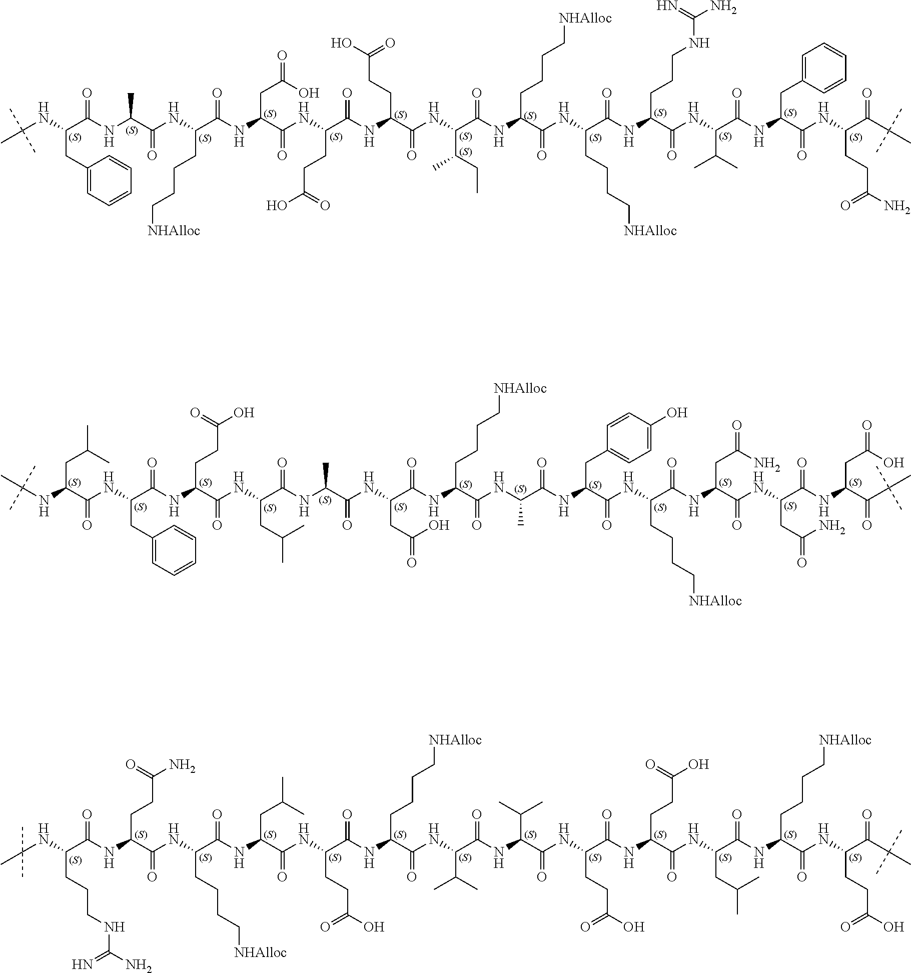

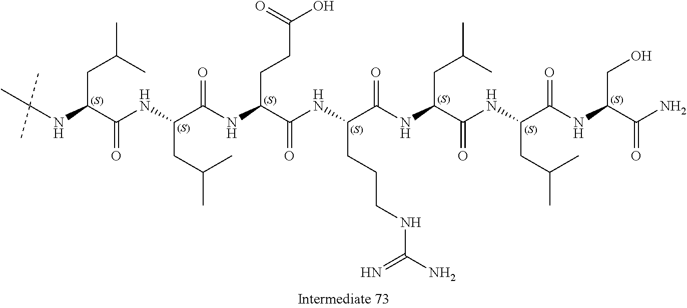

### Preparation of Compound 244

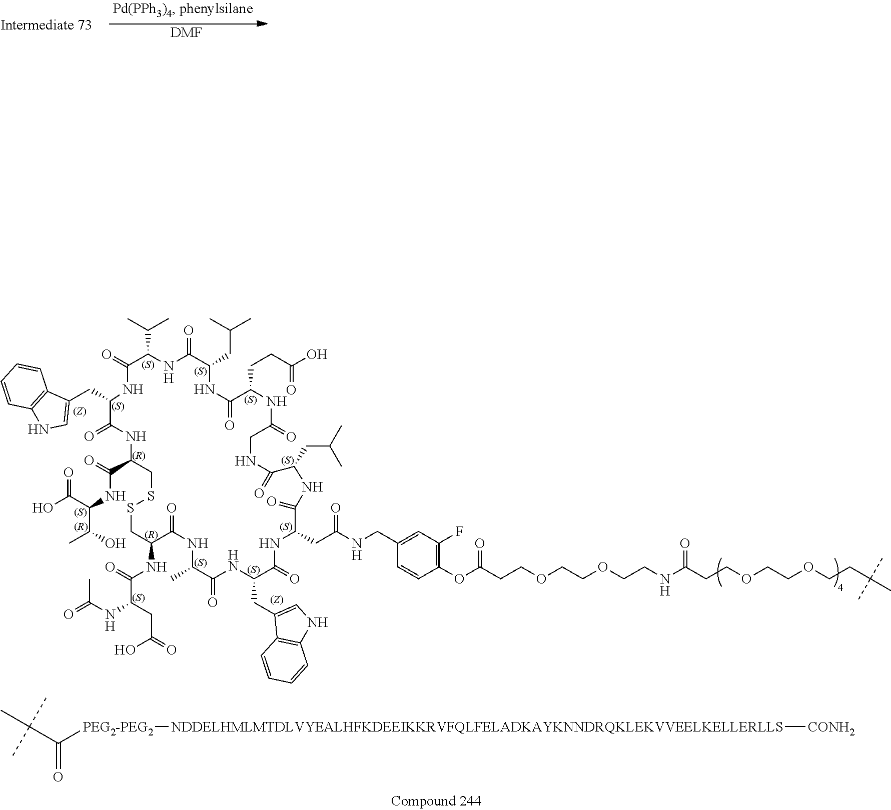

(SEQ ID NO: 107)
Reactive_uABT-PEG2-PEG8-PEG2-PEG2-
NDDELHMLMTDLVYEALHFAKDEEIKKRVFQLFELADKAYKNNDRQKLEK
VVEELKELLERLLS-NH2

A mixture of intermediate 73 (45.0 mg, 4.09 μmol, 1.00 equiv.), Pd(PPh₃)₄ (2.36 mg, 2.05 μmol, 0.50 equiv.), phenylsilane (8.7 mg, 80.70 μmol, 20.00 equiv.) in DMF (0.50 mL) was stirred under N₂ atmosphere at 20° C. for 1 h. Then the mixture was purified by prep-HPLC (A: 0.075% TFA/H₂O, B: MeCN) directly to afford Compound 244 (9.5 mg, 86.1% purity, 19.3% yield) as a white solid. LCMS: RT=1.10 min, MS cal.: 10321.67, mass observed: [M+7H]⁷⁺=1745.49, [M+8H]⁸⁺=1291.02, [M+10H]¹⁰⁺=1032.7.

### Example 24. Procedure for Preparation of Compound 245 (Linear Peptide+Cyclic Peptide)

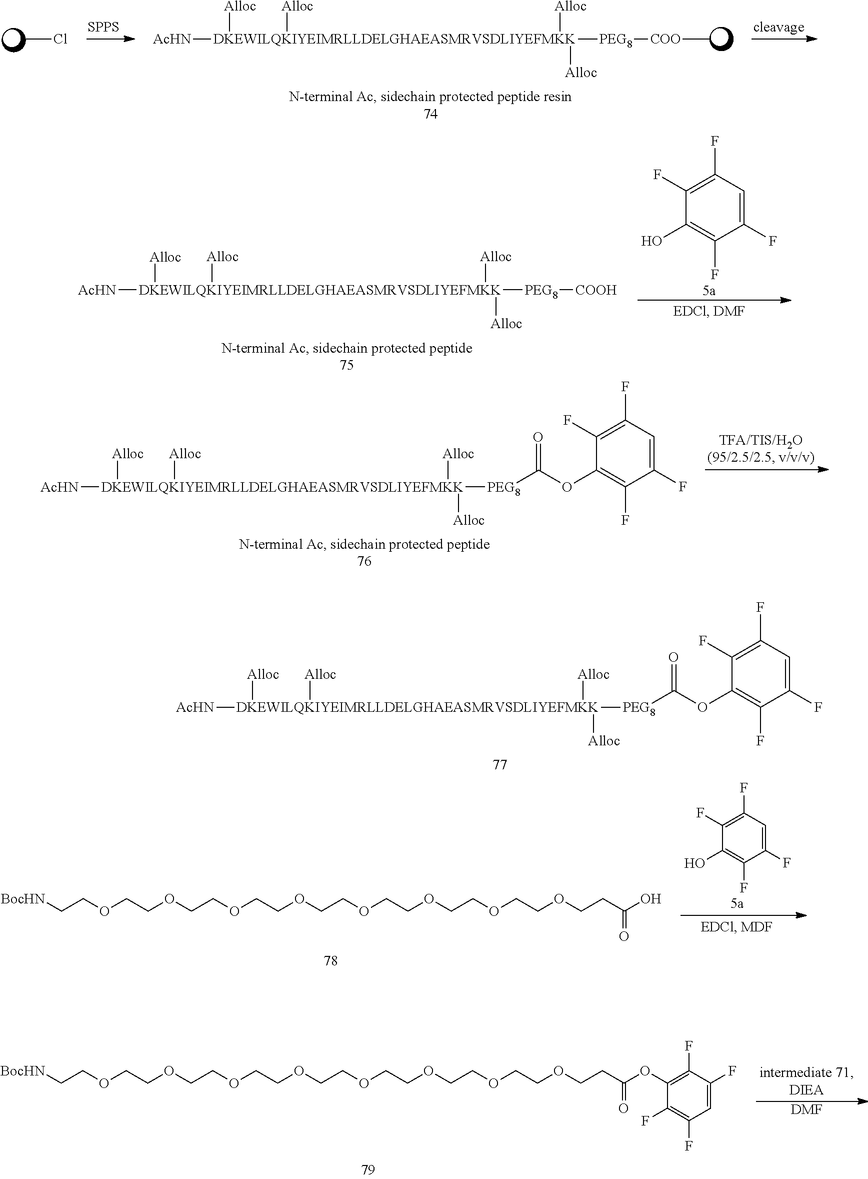

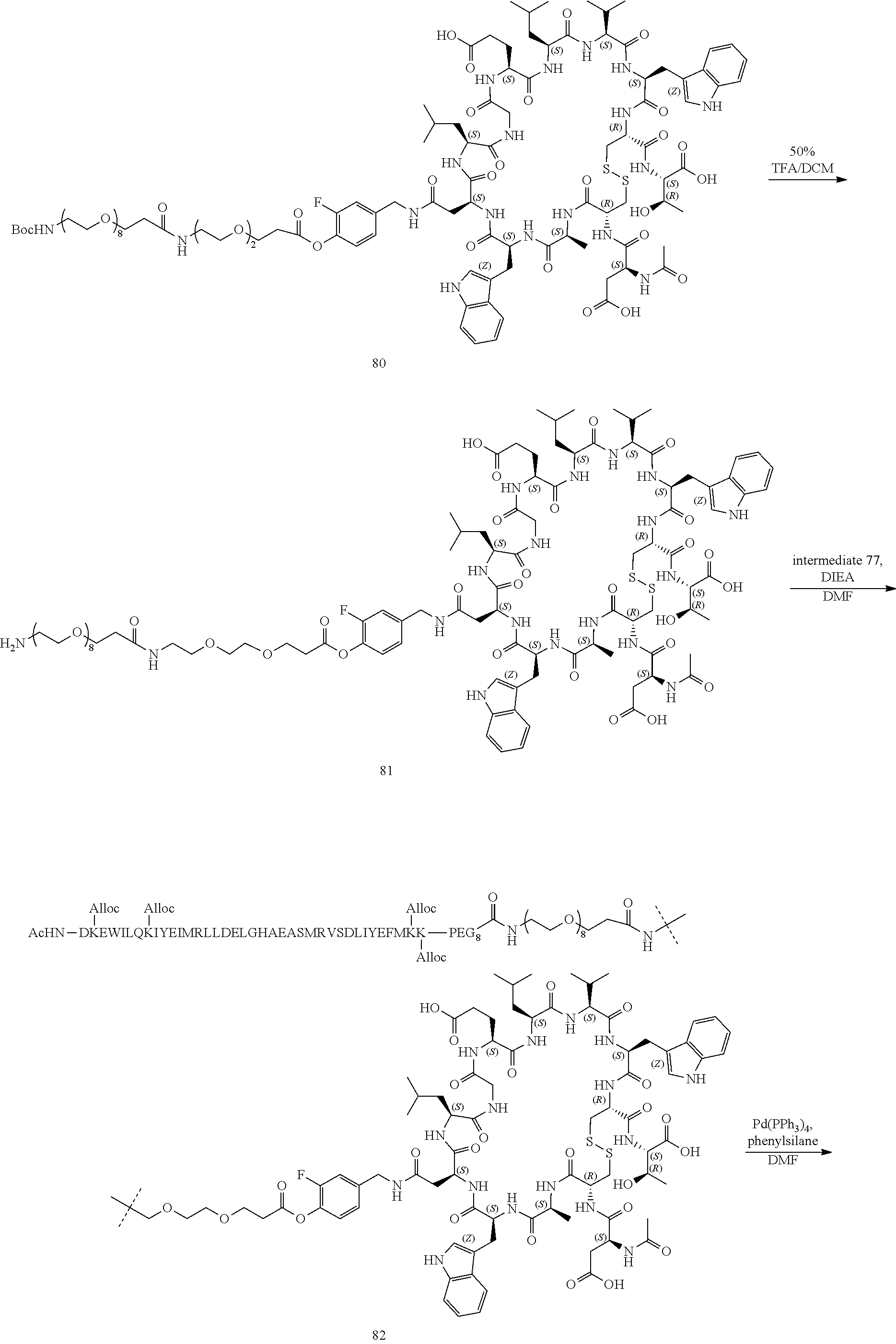

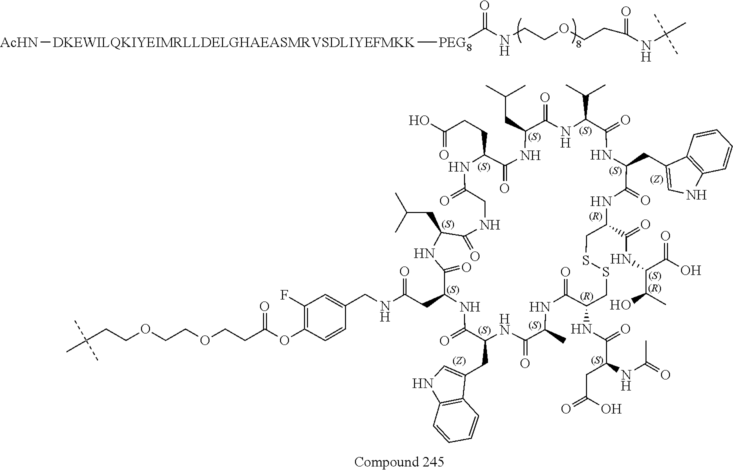

Preparation of Intermediate 75 (resin-bound peptide).

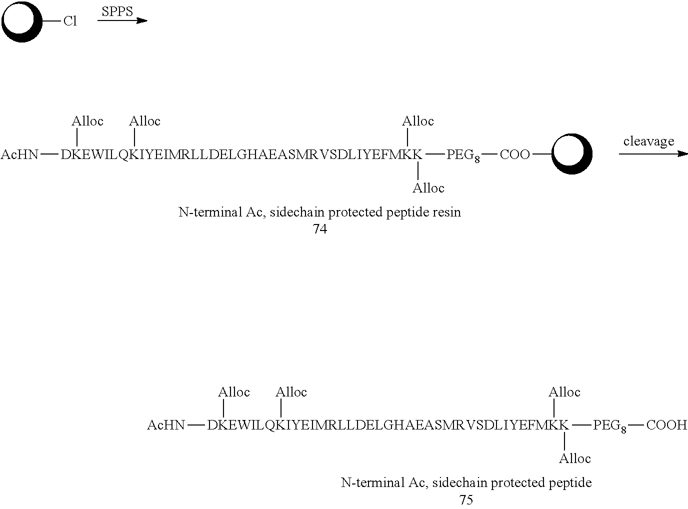

Peptide was synthesized using standard Fmoc chemistry (CTC resin).
1) Resin preparation: To the vessel containing CTC Resin (2.00 g, 2.00 mmol, 1.00 mmol/g) and Fmoc-PEG₈-OH (1.33 g, 2.00 mmol, 1.00 equiv.) in DCM (20 mL) was added DIEA (4.00 equiv.) dropwise and mixed for 2 h with N₂ bubbling at 20° C. Then MeOH (2.0 mL) was added and bubbled with N₂ for another 30 min. The resin was washed with DMF (40 mL)*5, followed by adding 20% piperidine in DMF (40 mL) and bubbled with N₂ for 30 mins at 20° C. for Fmoc deprotection. The mixture was filtered and the resin was washed with DMF (40 mL)*5 before proceeding to next step.
2) Coupling: A solution of Fmoc-Lys(Alloc)-OH (2.72 g, 3.00 equiv.), HBTU (2.16 g, 2.85 equiv.) in DMF (20 mL) was added to the resin with N₂ bubbling. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (40 mL)*5.
3) Deprotection: 20% piperidine in DMF (40 mL) was added to the resin and the mixture was bubbled with N₂ for 30 mins at 20° C. The deprotection reaction was monitored by ninhydrin test, if it showed blue or brownish red, the reaction was completed. The resin was then washed with DMF (40 mL) *5.
4) Steps 2 and 3 were repeated for the following amino acids: Number #3-17, Table 9.
5) Coupling: A solution of Fmoc-Ala-OH (1.87 g, 3.00 equiv.), HATU (2.16 g, 2.85 equiv.) in DMF (20 mL) was added to the resin with N₂ bubbling for 30 mins at 20° C. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (60 mL)*5.
6) Step 3 was repeated for Fmoc deprotection.
7) Steps 5 and 6 were repeated for the following amino acids elongation: Number #19□39, Table 19.
8) Acetylation: A solution of Ac₂O/NMM/DMF (2/1/17, v/v/v, 40 mL) was added to resin, the mixture was bubbled with N₂ for 20 min. The acetylation reaction was monitored by ninhydrin test. The resin was then washed with DMF (40 mL)*5, MeOH (40 mL)*3, then dried under reduced pressure.

TABLE 19
The list of amino acids and the corresponding reagents used on SPPS.

| # | Materials | Coupling reagents |
|---|---|---|
| 1 | Fmoc-PEG₈-OH (1.00 equiv.) | DIEA (4.00 equiv.) |
| 2 | Fmoc-Lys(Alloc)-OH (3.00 equiv.) | HBTU (2.85 equiv.) and DIEA (6.00 equiv.) |
| 3 | Fmoc-Lys(Alloc)-OH (3.00 equiv.) | HBTU (2.85 equiv.) and DIEA (6.00 equiv.) |
| 4 | Fmoc-Met-OH (3.00 equiv.) | HBTU (2.85 equiv.) and DIEA (6.00 equiv.) |
| 5 | Fmoc-Phe-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Tyr(tBu)-OH (3.00 equiv.) | HBTU (2.85 equiv.) and DIEA (6.00 equiv.) |
| 8 | Fmoc-Ile-OH (3.00 equiv.) | HBTU (2.85 equiv.) and DIEA (6.00 equiv.) |
| 9 | Fmoc-Leu-OH (3.00 equiv.) | HBTU (2.85 equiv.) and DIEA (6.00 equiv.) |
| 10 | Fmoc-Asp(OtBu)-OH (3.00 equiv.) | HBTU (2.85 equiv.) and DIEA (6.00 equiv.) |
| 11 | Fmoc-Ser(tBu)-OH (3.00 equiv.) | HBTU (2.85 equiv.) and DIEA (6.00 equiv.) |
| 12 | Fmoc-Val-OH (3.00 equiv.) | HBTU (2.85 equiv.) and DIEA (6.00 equiv.) |
| 13 | Fmoc-Arg(Pbf)-OH (3.00 equiv.) | HBTU (2.85 equiv.) and DIEA (6.00 equiv.) |
| 14 | Fmoc-Met-OH (3.00 equiv.) | HBTU (2.85 equiv.) and DIEA (6.00 equiv.) |
| 15 | Fmoc-Ser(tBu)-OH (3.00 equiv.) | HBTU (2.85 equiv.) and DIEA (6.00 equiv.) |
| 16 | Fmoc-Ala-OH (3.00 equiv.) | HBTU (2.85 equiv.) and DIEA (6.00 equiv.) |
| 17 | Fmoc-Glu(OtBu)-OH (3.00 equiv.) | HBTU (2.85 equiv.) and DIEA (6.00 equiv.) |
| 18 | Fmoc-Ala-OH (3.00 equiv.) | HATU (2.85 equiv.) and DIEA (6.00 equiv.) |
| 19 | Fmoc-His(Trt)-OH (3.00 equiv.) | HATU (2.85 equiv.) and DIEA (6.00 equiv.) |
| 20 | Fmoc-Gly-OH (3.00 equiv.) | HATU (2.85 equiv.) and DIEA (6.00 equiv.) |
| 21 | Fmoc-Leu-OH (3.00 equiv.) | HATU (2.85 equiv.) and DIEA (6.00 equiv.) |
| 22 | Fmoc-Glu(OtBu)-OH (3.00 equiv.) | HATU (2.85 equiv.) and DIEA (6.00 equiv.) |
| 23 | Fmoc-Asp(OtBu)-OH (3.00 equiv.) | HATU (2.85 equiv.) and DIEA (6.00 equiv.) |
| 24 | Fmoc-Leu-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Arg(Pbf)-OH (3.00 equiv.) | HATU (2.85 equiv.) and DIEA (6.00 equiv.) |
| 27 | Fmoc-Met-OH (3.00 equiv.) | HATU (2.85 equiv.) and DIEA (6.00 equiv.) |
| 28 | Fmoc-Ile-OH (3.00 equiv.) | HATU (2.85 equiv.) and DIEA (6.00 equiv.) |
| 29 | Fmoc-Glu(OtBu)-OH (3.00 equiv.) | HATU (2.85 equiv.) and DIEA (6.00 equiv.) |
| 30 | Fmoc-Tyr(tBu)-OH (3.00 equiv.) | HATU (2.85 equiv.) and DIEA (6.00 equiv.) |
| 31 | Fmoc-Ile-OH (3.00 equiv.) | HATU (2.85 equiv.) and DIEA (6.00 equiv.) |
| 32 | Fmoc-Lys(Alloc)-OH (3.00 equiv.) | HATU (2.85 equiv.) and DIEA (6.00 equiv.) |
| 33 | Fmoc-Gln(Trt)-OH (3.00 equiv.) | HATU (2.85 equiv.) and DIEA (6.00 equiv.) |
| 34 | Fmoc-Leu-OH (3.00 equiv.) | HATU (2.85 equiv.) and DIEA (6.00 equiv.) |
| 35 | Fmoc-Ile-OH (3.00 equiv.) | HATU (2.85 equiv.) and DIEA (6.00 equiv.) |
| 36 | Fmoc-Trp-OH (3.00 equiv.) | HATU (2.85 equiv.) and DIEA (6.00 equiv.) |
| 37 | Fmoc-Glu(OtBu)-OH (3.00 equiv.) | HATU (2.85 equiv.) and DIEA (6.00 equiv.) |
| 38 | Fmoc-Lys(Alloc)-OH (3.00 equiv.) | HATU (2.85 equiv.) and DIEA (6.00 equiv.) |
| 39 | Fmoc-Asp(OtBu)-OH (3.00 equiv.) | HATU (2.85 equiv.) and DIEA (6.00 equiv.) |
| 40 | Acetylation | Ac₂O/NMM/DMF (2/1/17, v/v/v, 40 mL) |

Peptide Cleavage: Cleavage solution (HFIP/DCM, 1/4, v/v, 100 mL) was added to the flask containing the side-chain protected resin-bound peptide (intermediate 74, SEQ ID NO:108, 15.1 g, 2.00 mmol) at room temperature and stirred for 1 h. After filtration, the filtrate was collected. The filtrate was concentrated under reduced pressure. The residue was added MeCN/H₂O (4/6, v/v, 500 mL) and lyophilized to afford intermediate 75 (SEQ ID NO:108) (12.0 g, crude) as a white solid.

### Preparation of Intermediate 76

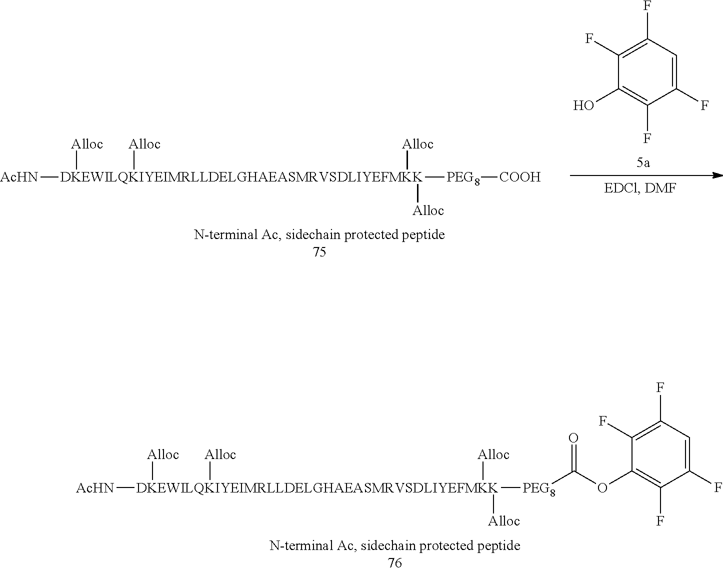

To a mixture of intermediate 75 (12.00 g, crude, 1.00 equiv.), intermediate 5a (3.32 g, 20.00 mmol, 10.00 equiv.) in DMF (100 mL) was added EDCI (2.30 g, 12 mmol, 6.00 equiv.) at 20° C. The reaction was stirred at 20° C. for 12 h. Then the mixture was precipitated with 0.1 M aq. HCl (cold, 1000 mL). After filtration, the solid was dried by lyophilization to afford intermediate 76 (18.30 g, crude) as a white solid.

### Preparation of Intermediate 77

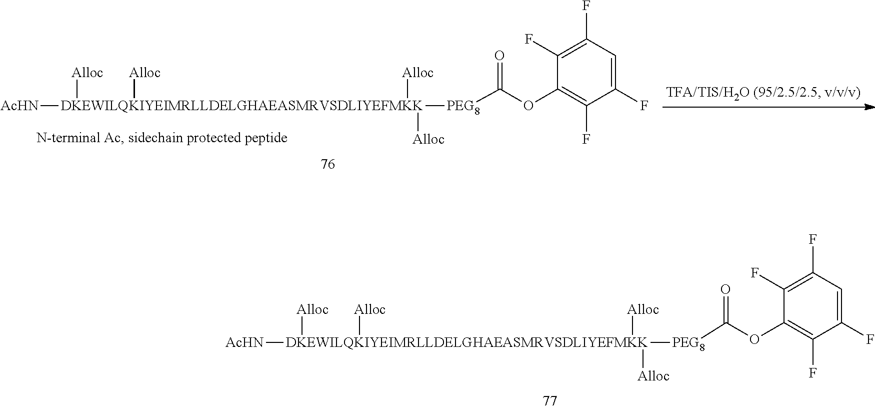

A solution of intermediate 76 (18.30 g, crude) in TFA/TIS/H₂O (95/2.5/2.5, v/v/v, 200 mL) was stirred at 20° C. for 2 h. The mixture was precipitated with cold isopropyl ether (800 mL). After filtration, the solid was washed with isopropyl ether (500 mL) twice, dried under reduced pressure at room temperature. The solid was purified by prep-HPLC (A: 0.075% TFA/H₂O; B: MeCN), followed by lyophilization to afford intermediate 77 (250.0 mg, 8.50% total yield, containing peptide with C-terminal free that TFP hydrolyzed) as a white solid.

### Preparation of Intermediate 79

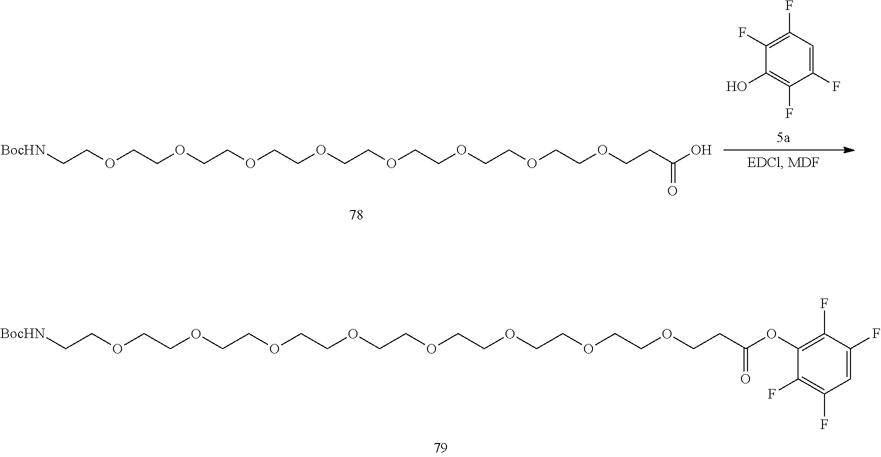 [SMILES: Oc1c(F)c(F)cc(F)c1F ; CC(C)(C)OC(=O)NCCOCCOCCOCCOCCOCCOCCOCCOCCC(=O)O ; CC(C)(C)OC(=O)NCCOCCOCCOCCOCCOCCOCCOCCOCCC(=O)Oc1c(F)c(F)cc(F)c1F]

To a solution of intermediate 78 1.00 g, 1.85 mmol, 1.00 equiv.) in DMF (10 mL) was added intermediate 5a (919.8 mg, 5.54 mmol, 3.00 equiv.), EDCI (530.91 mg, 2.77 mmol, 1.50 equiv.). The reaction was stirred at 20° C. for 16 h. The mixture was purified by prep-HPLC (A: 0.075% TFA/H₂O, B: MeCN) directly to afford intermediate 79 (1.10 g, 1.59 mmol, 86.39% yield) as a colorless oil. LCMS: RT=1.142 min, MS cal.: 689.69, mass observed: [M+H]⁺=690.1, [M+H₂O+H]⁺=707.2, [M-Boc+H]⁺=590.1, [M-tBu+H]⁺=634.1.

### Preparation of Intermediate 80

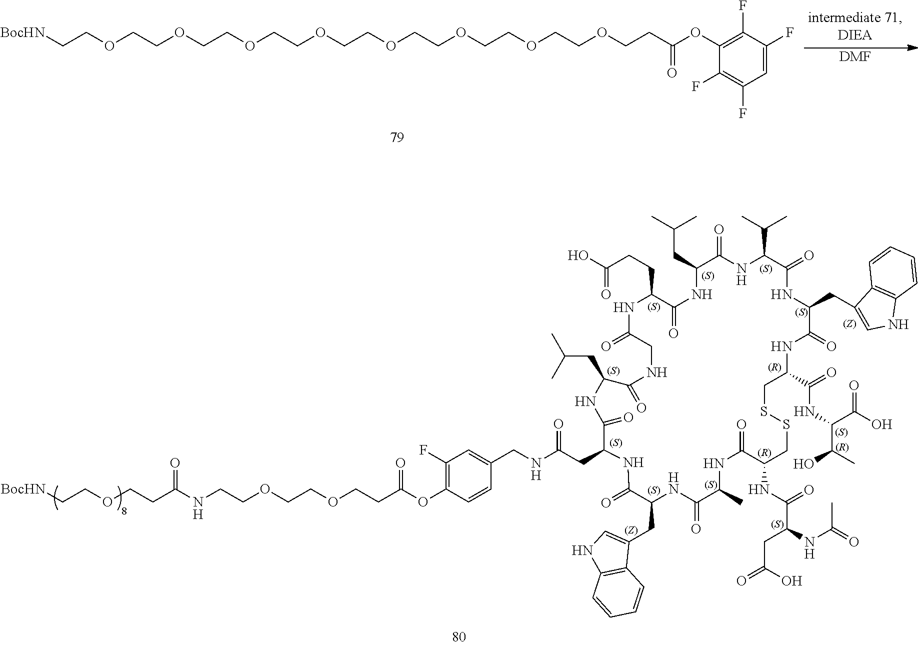

A mixture of intermediate 71 (300.0 mg, 163.66 μmol, 1.00 equiv.), intermediate 79 (112.88 mg, 163.66 μmol, 1.00 equiv.), DIEA (105.76 mg, 818.32 μmol, 142.54 uL, 5.00 equiv.) in DMSO (9 mL) was stirred at 20° C. for 0.5 h. The mixture was purified by prep-HPLC (A: 0.075% TFA/H₂O, B: MeCN) directly to afford intermediate 80 (200.0 mg, 84.87 μmol, 51.8% yield) as a white solid. LCMS: RT=1.057 min, MS cal.: 2356.63, mass observed: [M+2H]²⁺=1179.10.

### Preparation of Intermediate 81

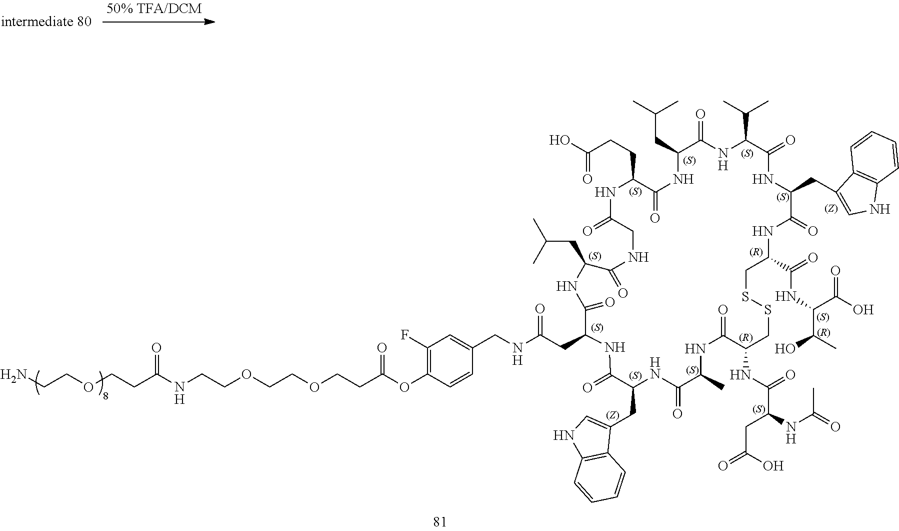

A mixture of intermediate 80 (200.0 mg, 84.87 μmol, 1.00 equiv.) in TFA (1.54 g, 13.51 mmol, 1 mL, 159.14 equiv.), DCM (3 mL) was stirred at 0° C. for 0.5 h. The solvent was removed under reduced pressure, the residue was lyophilizated to afford intermediate 81 (200 mg, 84.37 μmol, 99.41% yield, TFA salt) as a white solid. LCMS: RT=0.933 min, MS cal.: 2256.52, mass observed: [M+2H]²⁺=1128.79, [M+3H]³⁺=752.89, [M+4H]⁴⁺+=564.90.

### Preparation of Intermediate 82

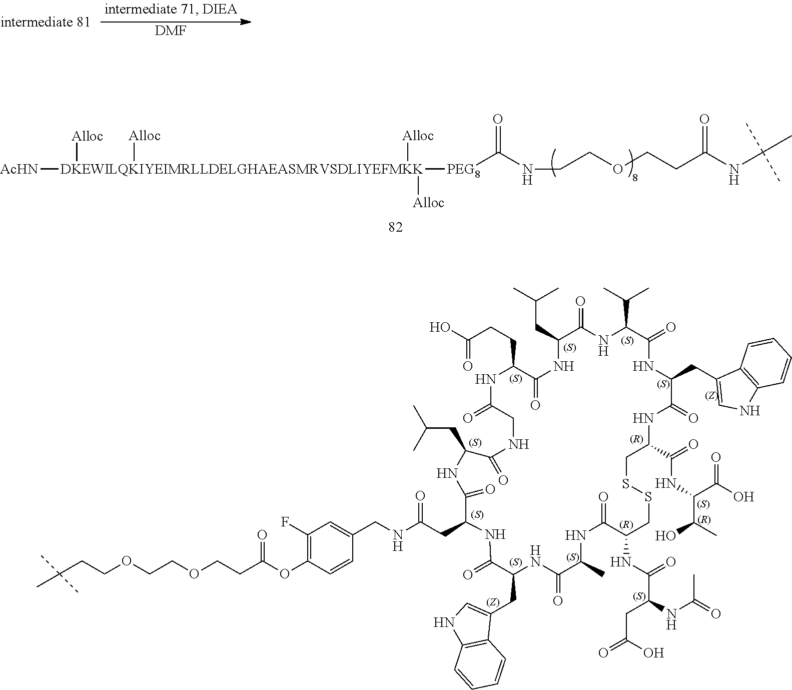

To a solution of intermediate 77 (150.24 mg, 26.59 μmol, 1.20 equiv.), intermediate 81 (50.0 mg, 22.16 μmol, 1.00 equiv.) in DMF (2.00 mL) was added DIEA (17.18 mg, 132.95 μmol, 23.16 uL, 6.00 equiv.) to pH=8. Then the mixture was stirred at 20° C. for 0.5 h. The mixture was purified by prep-HPLC (A: 0.075% TFA/H₂O, B: MeCN) to afford intermediate 82 (90.0 mg, 11.63 μmol, 52.47% yield) as a white solid. LCMS: RT=0.933 min, MS cal.: 7740.79, mass observed: [M+4H]⁴⁺=1935.93, [M+5H]⁵⁺=1549.09, [M+6H]⁶⁺=1290.91, [M+7H]⁷⁺=1106.71, [M+8H]⁸⁺=968.49.

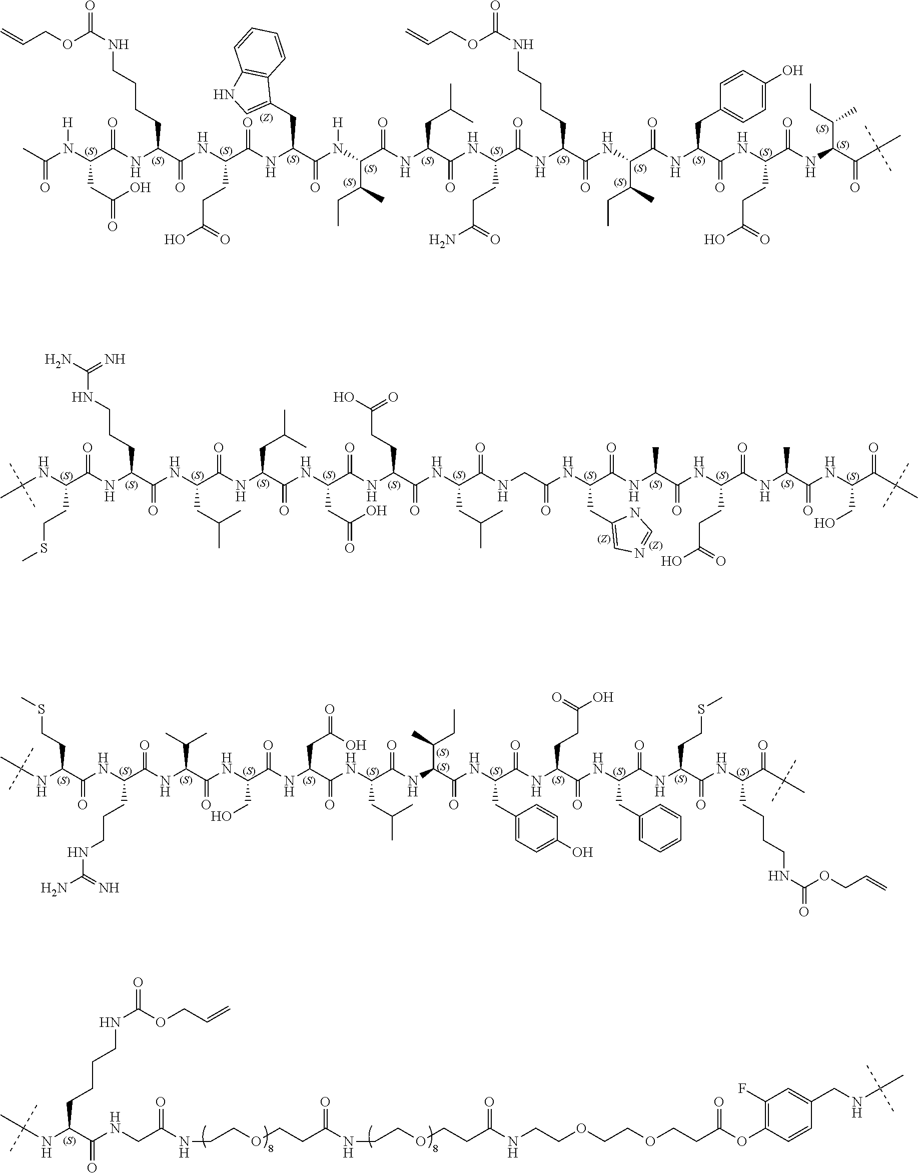

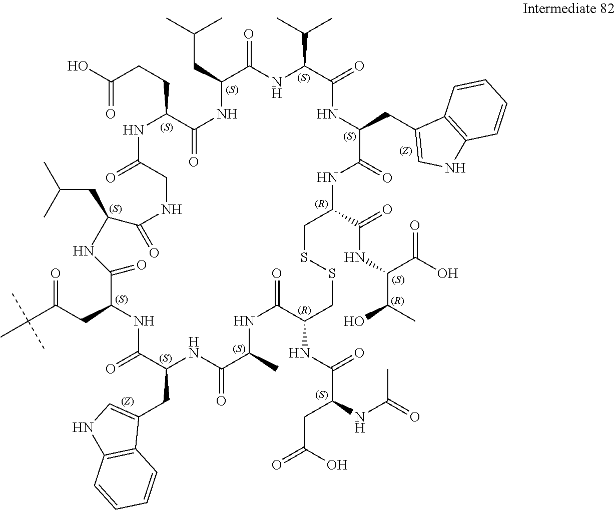

### Preparation of Compound 245

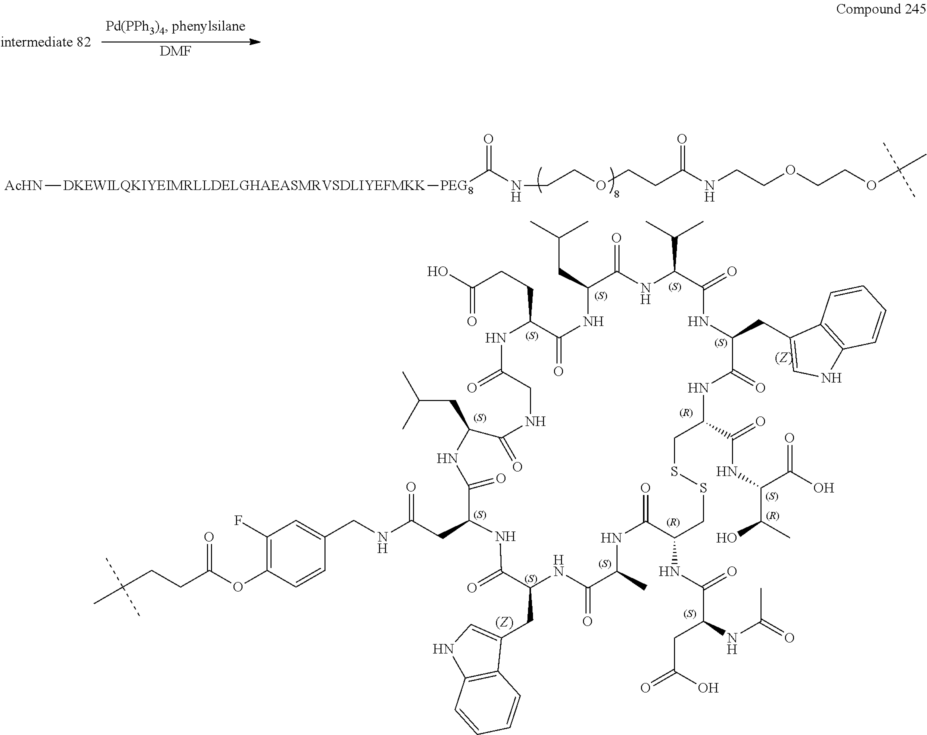

To a solution of intermediate 82 (12.58 mg, 116.27 μmol, 14.35 μL, 10.00 equiv.), Pd(PPh₃)₄ (6.72 mg, 5.81 μmol, 0.50 equiv.) in DMF (1 mL) was stirred under N₂ atmosphere at 20° C. for 1 h. The mixture was purified by prep-HPLC (A: 0.075% TFA/H₂O, B: MeCN) directly to afford Compound 245 (Batch 1, 11.4 mg, 95.1% purity; Batch 2, 25.2 mg, 95.7% purity; 40.43% yield) as a white solid. LCMS: RT=0.933 min, MS cal.: 7740.79, mass observed: [M+4H]⁴⁺+=1853.00, [M+5H]⁵⁺=1482.50, [M+6H]⁶⁺=1235.5, [M+7H]⁷⁺=1059.1, [M+8H]⁸⁺=926.9.

Compounds 246, 247, 248, 249, and 250 were synthesized using the same method Compound 245, above.

0.25 mmol resin afforded Compound 246 (24.9 mg, 98.7% purity, 1.42% yield) as a white solid. LCMS: RT=1.961 min, MS cal.: 6981.00, mass observed: [M+4H]⁴⁺+=1746.60, [M+5H]⁵⁺=1397.70, [M+6H]⁶⁺=1164.90, [M+7H]⁷⁺=998.80, [M+8H]⁸⁺=873.90.

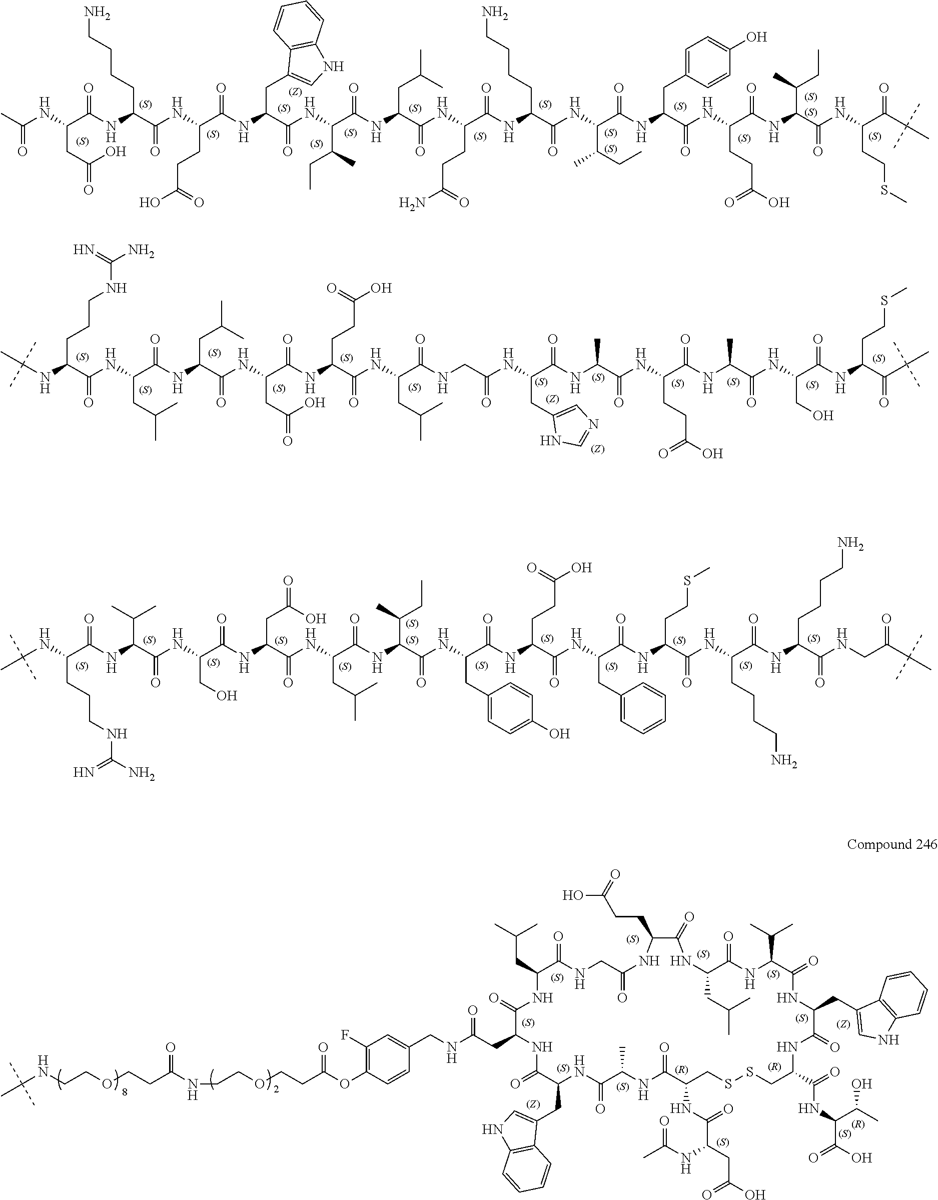

0.25 mmol resin afforded Compound 247 (20.5 mg, 93.8% purity, 1.17% yield) as a white solid. LCMS: RT=1.961 min, MS cal.: 6557.50, mass observed: [M+4H]⁴⁺=1640.90, [M+5H]⁵⁺=1313.20, [M+6H]⁶⁺=1094.40, [M+7H]⁷⁺=937.70.

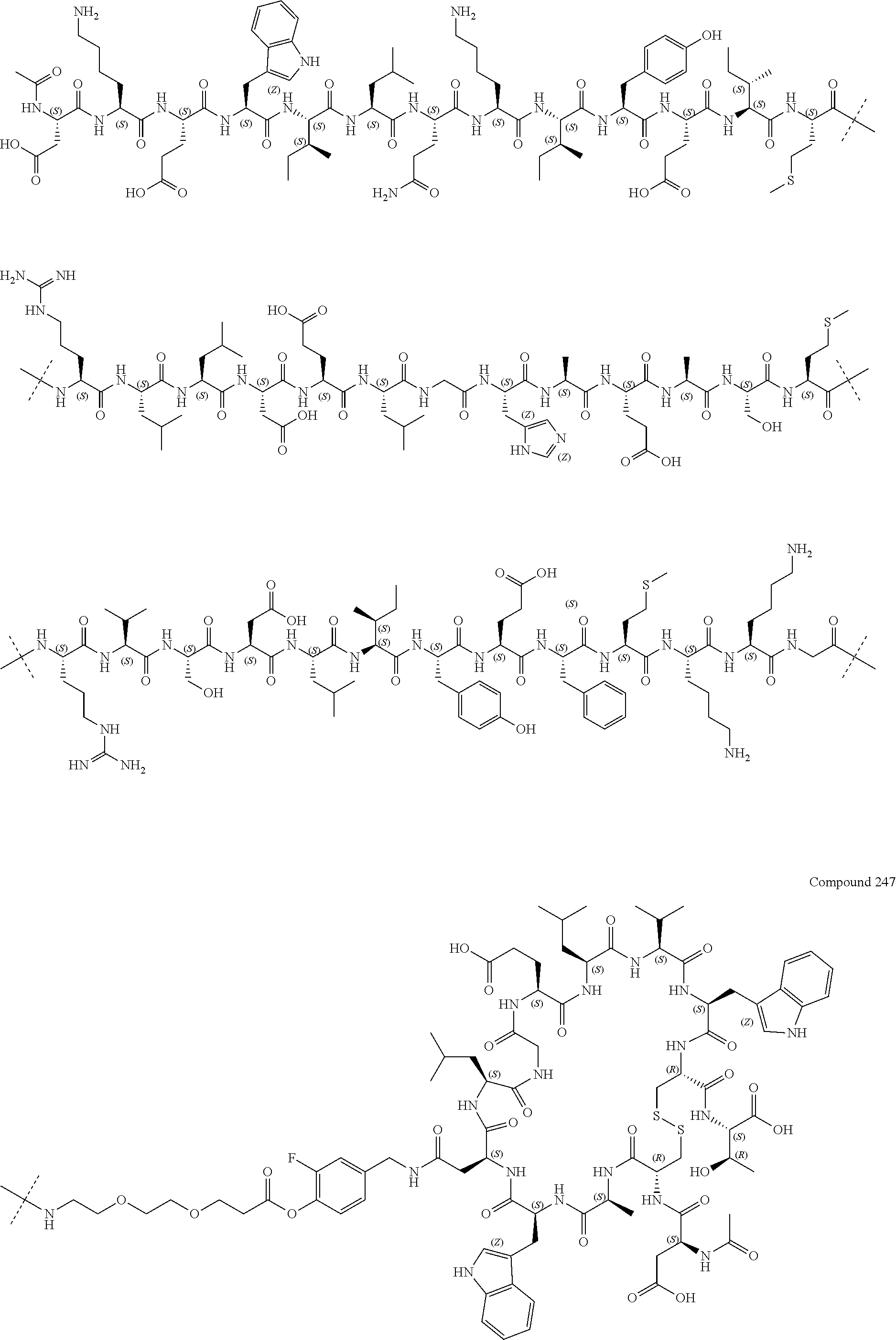

0.25 mmol resin afforded Compound 248 (5.6 mg, 85.8% purity, 0.27% yield) as a white solid. LCMS: RT=1.961 min, MS cal.: 6966.97, mass observed: [M+4H]⁴⁺=1742.79, [M+5H]⁵⁺=1394.42.

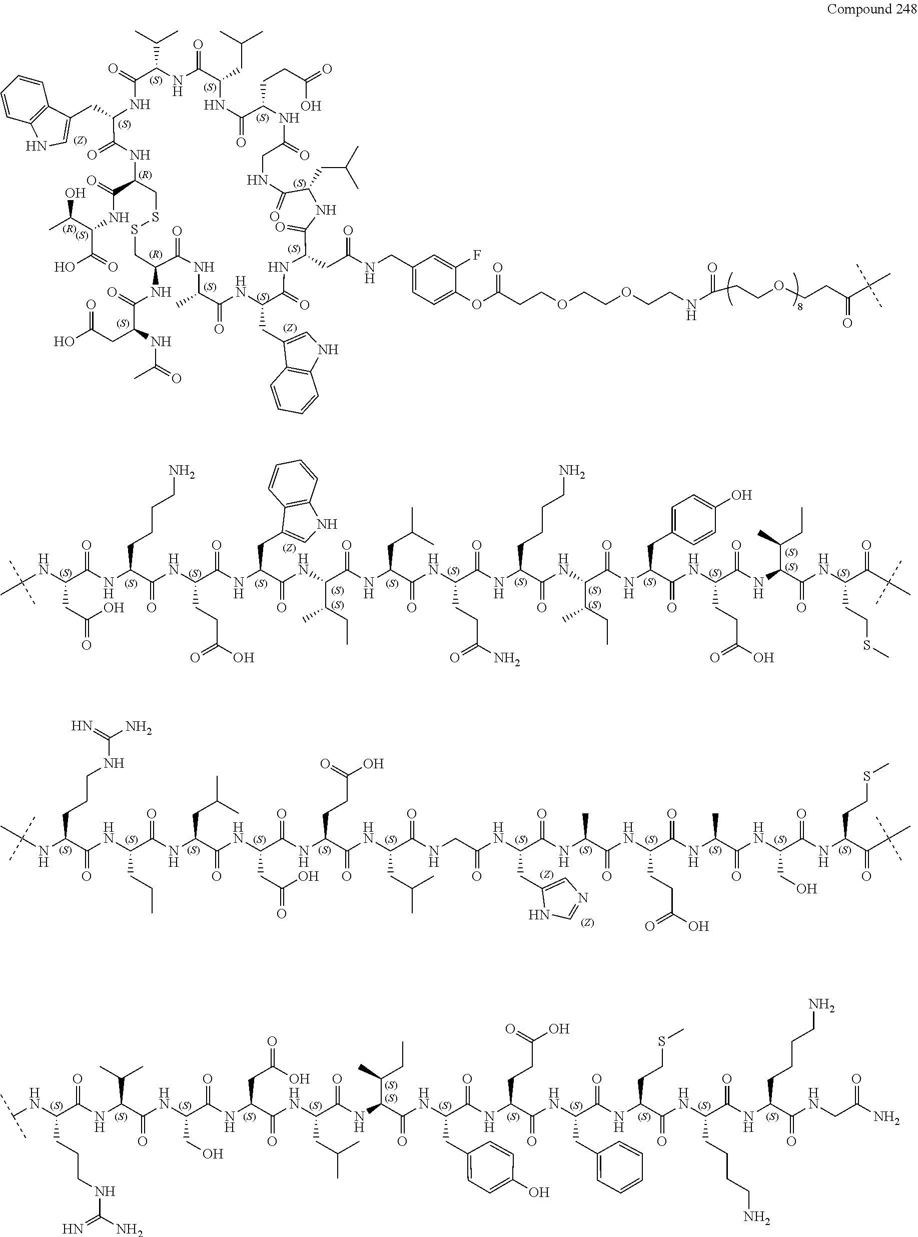

0.25 mmol resin afforded Compound 249 (10.2 mg, 96.9% purity, 0.27% yield) as a white solid. LCMS: RT=1.945 min, MS cal.: 6812.73, mass observed: [M+4H]⁴⁺=1704.90, [M+5H]⁵⁺=1364.10, [M+6H]⁶⁺=1136.80, [M+7H]⁷⁺=974.80.

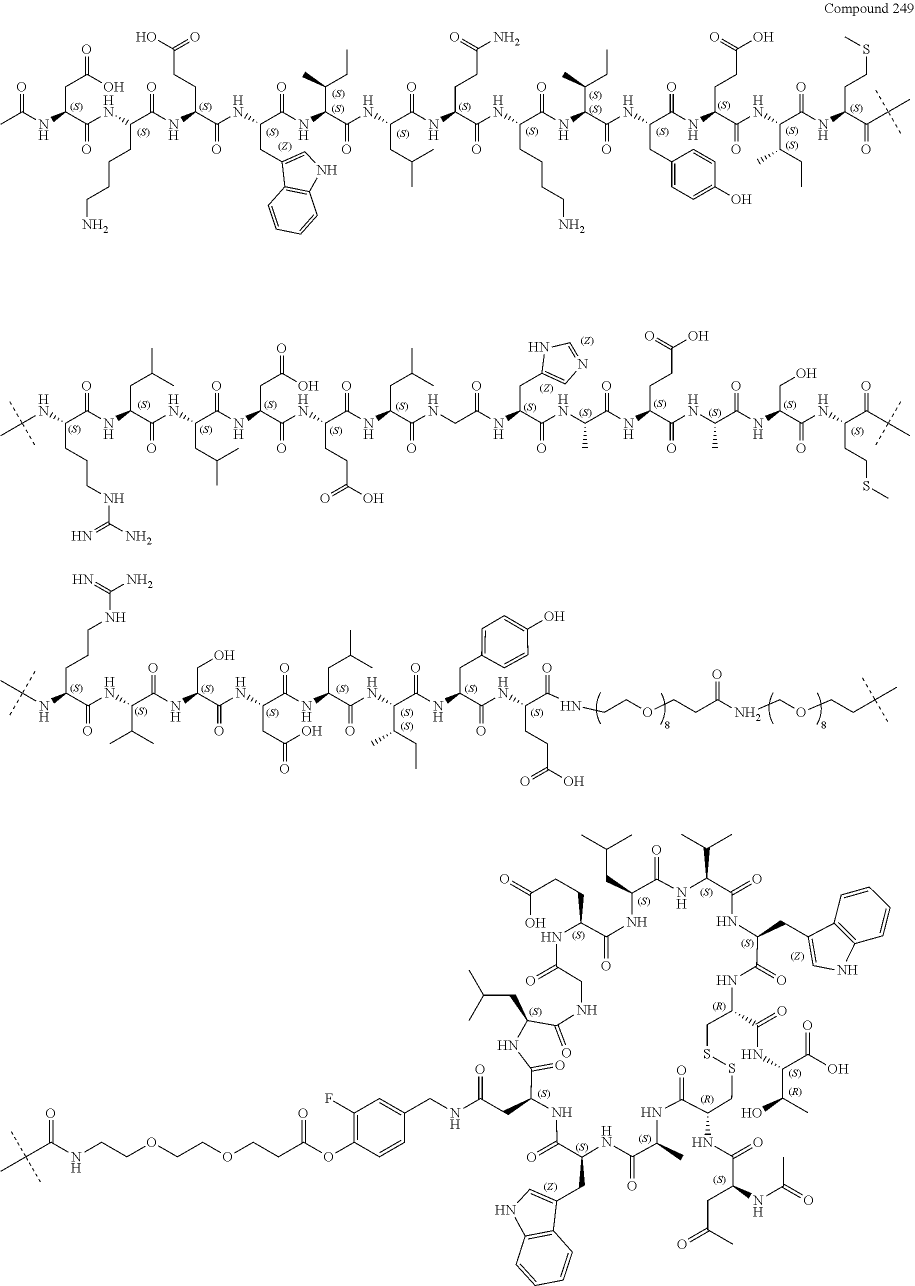

0.25 mmol resin afforded Compound 250 (21.5 mg, 87.4% purity, 0.27% yield) as a white solid. LCMS: RT=2.011 min, MS cal.: 6845.91, mass observed: [M+4H]⁴⁺=1713. 10, [M+5H]⁵⁺=1370.60, [M+6H]⁶⁺=1142.40.

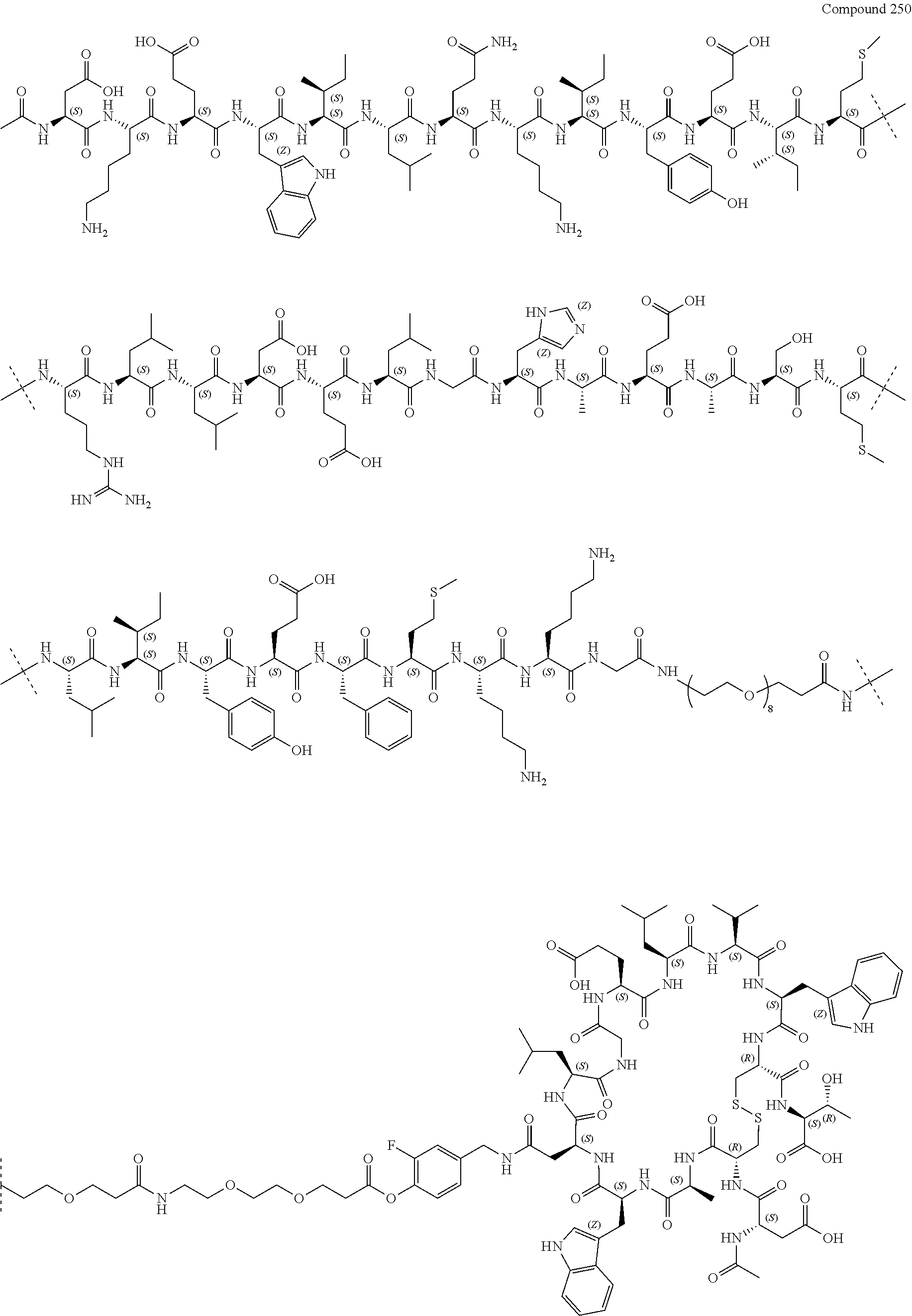

### Example 25. Procedure for Preparation of Compound 251-Strata 1.5-Biotin

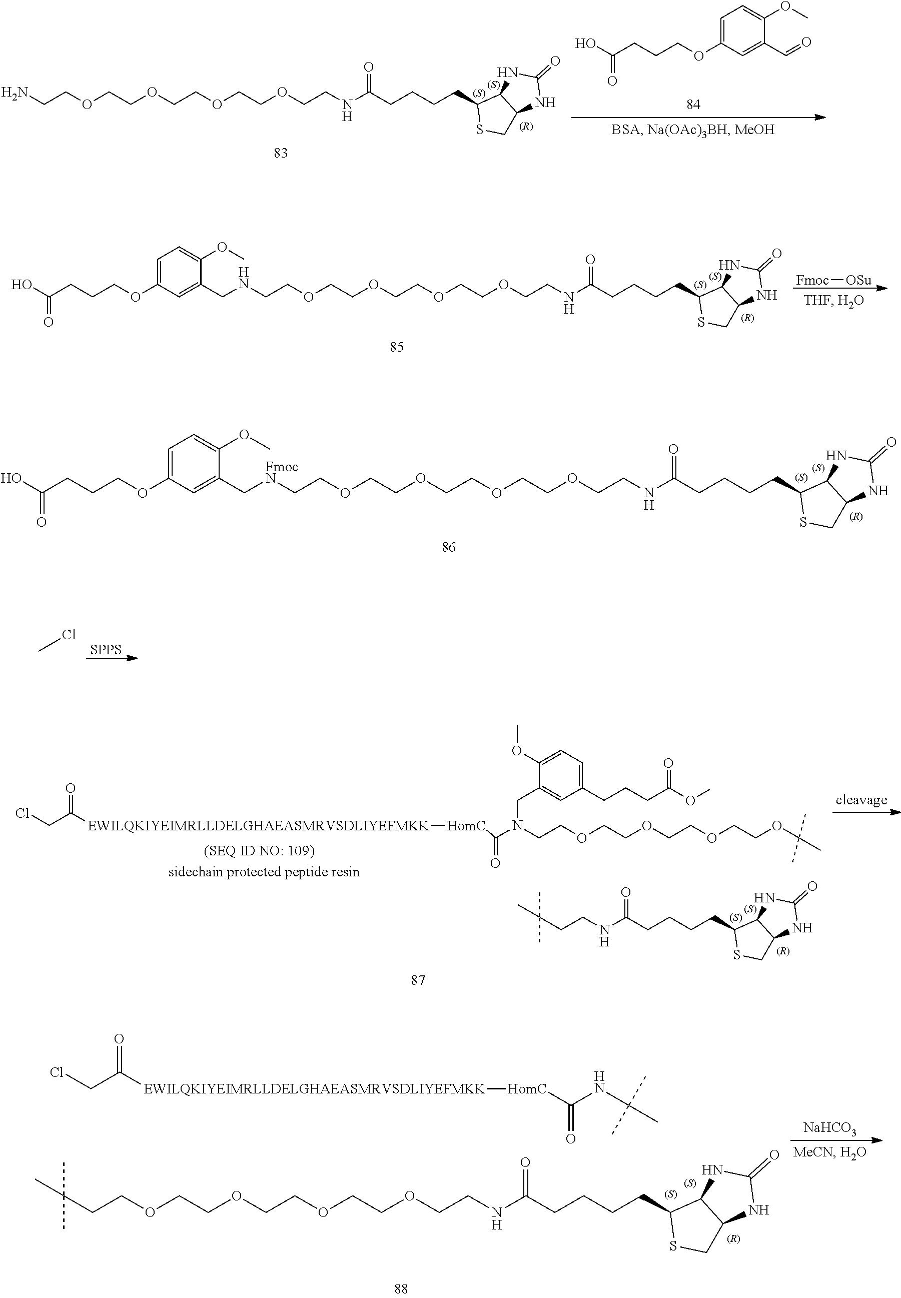

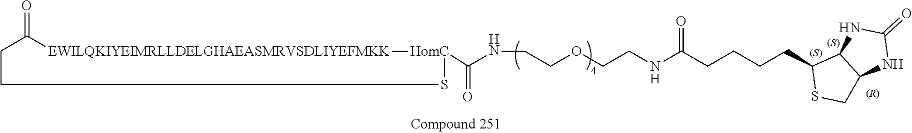

### Preparation of Intermediate 85

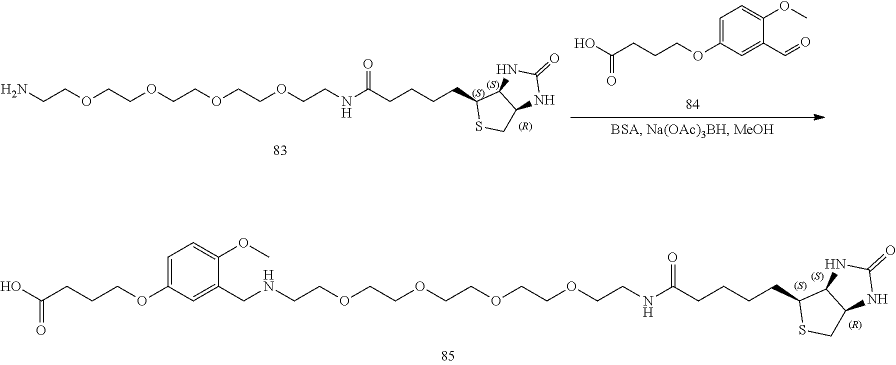

To a solution of intermediate 84 (308.9 mg, 1.30 mmol, 1.20 equiv.) in THF (3.0 mL) was added intermediate 83 (500.0 mg, 1.08 mmol, 1.00 equiv.), NaBH(OAc)₃ (343.6 mg, 1.62 mmol, 1.50 equiv.) and BSA/scandium(3+);trifluoromethanesulfonate (531.9 mg, 1.08 mmol, 1.00 equiv.), then the reaction mixture was stirred at 20° C. for 2 h. The mixture was quenched with H₂O (0.1 mL). Then the mixture was purified by prep-HPLC (A: 0.075% TFA/H₂O, B: MeCN) to afford intermediate 85 (420.0 mg, 613.28 μmol, 56.7% yield) as white solid. LCMS: RT=0.759 min, MS cal.: 684.84, mass observed: [M+H]⁺=685.4.

### Preparation of Intermediate 86

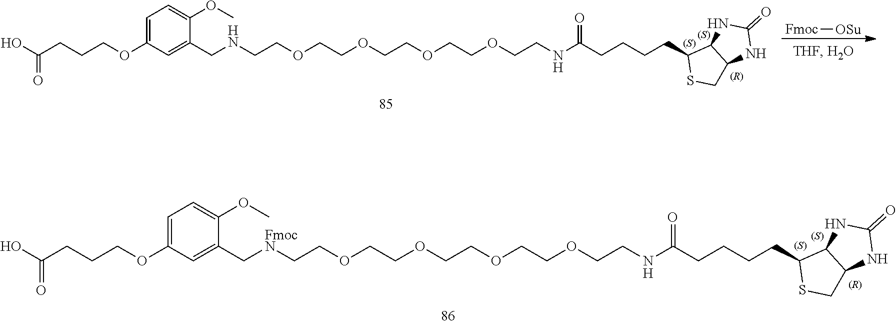

To a solution of Intermediate 85 (420.0 mg, 613.2 μmol, 1.00 equiv.), NaHCO₃ (154.5 mg, 1.83 mmol, 3.00 equiv.) in THF (5 mL) and H₂O (5 mL) was added Fmoc-OSu (310.3 mg, 919.9 μmol, 1.50 equiv.) at 20° C. Then the mixture was stirred at 20° C. for 3 h. The THF was removed under reduced pressure. The residue was acidified by 0.5 HCl (cold) to pH=6. The mixture was purified by prep-HPLC (A: 0.075% TFA/H₂O, B: MeCN) to afford intermediate 86 (356.0 mg, 392.47 μmol, 63.9% yield) as white solid.

### Preparation of Compound 251-Strata Biotin 1.5

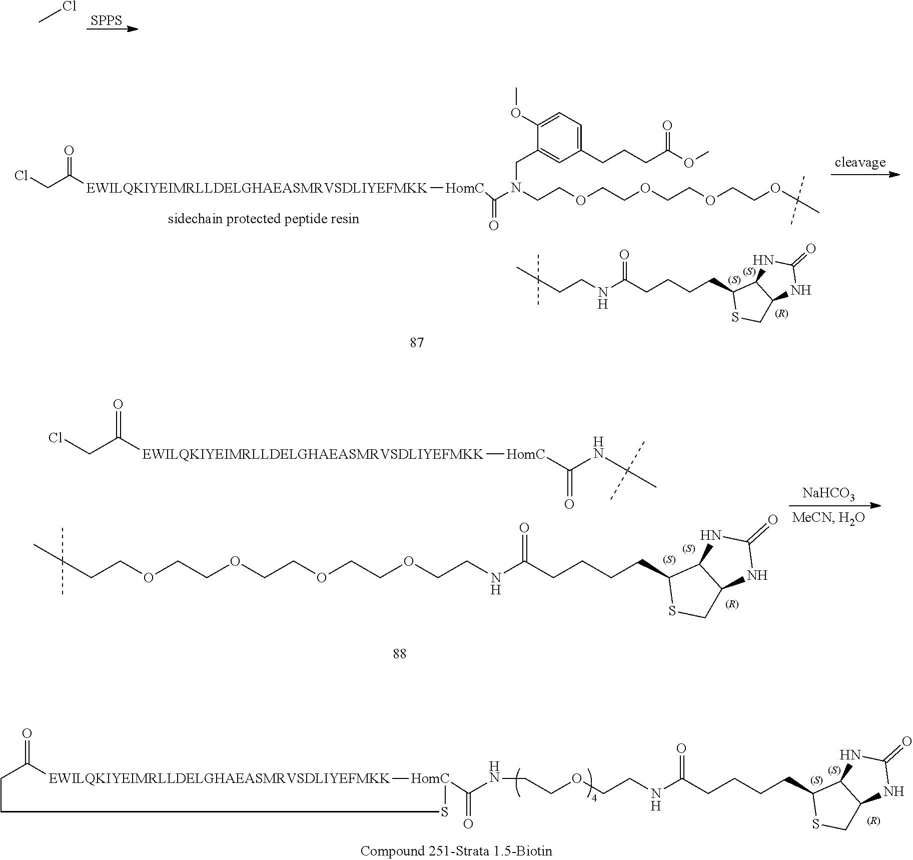

Peptide was synthesized using standard Fmoc chemistry (CTC resin).

Resin preparation: To the vessel containing CTC Resin (0.20 g, 0.20 mmol, 1.00 mmol/g) and intermediate 86 (181.4 mg, 0.20 mmol, 1.00 equiv.) in DCM (2 mL) was added DIEA (4.00 equiv.) dropwise and mixed for 2 h with N₂ bubbling at 20° C. Then MeOH (0.2 mL) was added and bubbled with N₂ for another 30 min. The resin was washed with DMF (5 mL)*5, followed by adding 20% piperidine in DMF (10 mL) and bubbled with N₂ for 30 min at 20° C. for Fmoc deprotection. The mixture was filtered and the resin was washed with DMF (5 mL)*5 before proceeding to next step.

Coupling: A solution of Fmoc-HomCys(Trt)-OH (214.2 mg, 3.00 equiv.), HATU (548.6 mg, 2.85 equiv.) in DMF (2 mL) was added to the resin with N₂ bubbling for 30 min at 20° C. Then DIEA (6.00 equiv.) was added to the mixture dropwise and bubbled with N₂ for 30 mins at 20° C. The coupling reaction was monitored by ninhydrin test, if it showed colorless, the coupling was completed. The resin was then washed with DMF (5 mL)*5.

Deprotection: 20% piperidine in DMF (10 mL) was added to the resin and the mixture was bubbled with N₂ for 30 mins at 20° C. The deprotection reaction was monitored by ninhydrin test, if it showed blue or brownish red, the reaction was completed. The resin was then washed with DMF (10 mL) *5.

The coupling and deprotection steps were repeated for the following amino acids elongation: Number #2-38, Table 20.

Coupling N-(Chloroacetoxy)succinimide: A solution of N-(Chloroacetoxy)succinimide (229.2 mg, 6.00 equiv.), DIEA (309.6 mg, 12.00 equiv.) in DMF (2 mL) was added to resin, the mixture was bubbled with N₂ for 20 min. The acetylation reaction was monitored by ninhydrin test. The resin was then washed with DMF (10 mL)*5, MeOH (10 mL)*3, then dried under reduced pressure to afford intermediate 87 (CTC resin, 1.80 g, 0.20 mmol).

TABLE 20
The list of amino acids and the corresponding reagents used on SPPS.

| # | Materials | Coupling reagents |
|---|---|---|
| 1 | intermediate 86 (1.00 equiv.) | DIEA (4.00 equiv.) |
| 2 | Fmoc-HomCys(Trt)-OH (3.00 equiv.) | HATU (2.85 equiv.) and DIEA (6.00 equiv.) |
| 3 | Fmoc-Lys(Boc)-OH (3.00 equiv.) | HATU (2.85 equiv.) and DIEA (6.00 equiv.) |
| 4 | Fmoc-Lys(Boc)-OH (3.00 equiv.) | HATU (2.85 equiv.) and DIEA (6.00 equiv.) |
| 5 | Fmoc-Met-OH (3.00 equiv.) | HATU (2.85 equiv.) and DIEA (6.00 equiv.) |
| 6 | Fmoc-Phe-OH (3.00 equiv.) | HATU (2.85 equiv.) and DIEA (6.00 equiv.) |
| 7 | Fmoc-Glu(OtBu)-OH (3.00 equiv.) | HATU (2.85 equiv.) and DIEA (6.00 equiv.) |
| 8 | Fmoc-Tyr(tBu)-OH (3.00 equiv.) | HATU (2.85 equiv.) and DIEA (6.00 equiv.) |
| 9 | Fmoc-Ile-OH (3.00 equiv.) | HATU (2.85 equiv.) and DIEA (6.00 equiv.) |
| 10 | Fmoc-Leu-OH (3.00 equiv.) | HATU (2.85 equiv.) and DIEA (6.00 equiv.) |
| 11 | Fmoc-Asp(OtBu)-OH (3.00 equiv.) | HATU (2.85 equiv.) and DIEA (6.00 equiv.) |
| 12 | Fmoc-Ser(OtBu)-OH (3.00 equiv.) | HATU (2.85 equiv.) and DIEA (6.00 equiv.) |
| 13 | Fmoc-Val-OH (3.00 equiv.) | HATU (2.85 equiv.) and DIEA (6.00 equiv.) |
| 14 | Fmoc-Arg(Pbf)-OH (3.00 equiv.) | HATU (2.85 equiv.) and DIEA (6.00 equiv.) |
| 15 | Fmoc-Met-OH (3.00 equiv.) | HATU (2.85 equiv.) and DIEA (6.00 equiv.) |
| 16 | Fmoc-Ser(tBu)-OH (3.00 equiv.) | HATU (2.85 equiv.) and DIEA (6.00 equiv.) |
| 17 | Fmoc-Ala-OH (3.00 equiv.) | HATU (2.85 equiv.) and DIEA (6.00 equiv.) |
| 18 | Fmoc-Glu(OtBu)-OH (3.00 equiv.) | HATU (2.85 equiv.) and DIEA (6.00 equiv.) |
| 19 | Fmoc-Ala-OH (3.00 equiv.) | HATU (2.85 equiv.) and DIEA (6.00 equiv.) |
| 20 | Fmoc-His(Trt)-OH (3.00 equiv.) | HATU (2.85 equiv.) and DIEA (6.00 equiv.) |
| 21 | Fmoc-Gly-OH (3.00 equiv.) | HATU (2.85 equiv.) and DIEA (6.00 equiv.) |
| 22 | Fmoc-Leu-OH (3.00 equiv.) | HATU (2.85 equiv.) and DIEA (6.00 equiv.) |
| 23 | Fmoc-Glu(OtBu)-OH (3.00 equiv.) | HATU (2.85 equiv.) and DIEA (6.00 equiv.) |
| 24 | Fmoc-Asp(OtBu)-OH (3.00 equiv.) | HATU (2.85 equiv.) and DIEA (6.00 equiv.) |
| 25 | Fmoc-Leu-OH (3.00 equiv.) | HATU (2.85 equiv.) and DIEA (6.00 equiv.) |
| 26 | Fmoc-Leu-OH (3.00 equiv.) | HATU (2.85 equiv.) and DIEA (6.00 equiv.) |
| 27 | Fmoc-Arg(Pbf)-OH (3.00 equiv.) | HATU (2.85 equiv.) and DIEA (6.00 equiv.) |
| 28 | Fmoc-Met-OH (3.00 equiv.) | HATU (2.85 equiv.) and DIEA (6.00 equiv.) |
| 29 | Fmoc-Ile-OH (3.00 equiv.) | HATU (2.85 equiv.) and DIEA (6.00 equiv.) |
| 30 | Fmoc-Glu(OtBu)-OH (3.00 equiv.) | HATU (2.85 equiv.) and DIEA (6.00 equiv.) |
| 31 | Fmoc-Tyr(tBu)-OH (3.00 equiv.) | HATU (2.85 equiv.) and DIEA (6.00 equiv.) |
| 32 | Fmoc-Ile-OH (3.00 equiv.) | HATU (2.85 equiv.) and DIEA (6.00 equiv.) |
| 33 | Fmoc-Lys(Boc)-OH (3.00 equiv.) | HATU (2.85 equiv.) and DIEA (6.00 equiv.) |
| 34 | Fmoc-Gln(Trt)-OH (3.00 equiv.) | HATU (2.85 equiv.) and DIEA (6.00 equiv.) |
| 35 | Fmoc-Leu-OH (6.00 equiv.) | HATU (5.70 equiv.) and DIEA (12.00 equiv.) |
| 36 | Fmoc-Ile-OH (6.00 equiv.) | HATU (5.70 equiv.) and DIEA (12.00 equiv.) |
| 37 | Fmoc-Trp(Boc)-OH (6.00 equiv.) | HATU (5.70 equiv.) and DIEA (12.00 equiv.) |
| 38 | Fmoc-Glu(OtBu)-OH (6.00 equiv.) | HATU (5.70 equiv.) and DIEA (12.00 equiv.) |
| 39 | N-(Chloroacetoxy)succinimide (6.00 equiv.) | DIEA (12.00 equiv.) |

Peptide cleavage, cyclization and purification: Cleavage solution (TFA/TIS/H₂O, 95/2.5/2.5, v/v/v, 20 mL) was added to the flask containing the side-chain protected resin-bound peptide (intermediate 87, 1.80 g, 0.20 mmol) at room temperature and stirred for 1 h.

After filtration, the filtrate was collected.

The filtrate was precipitated with cold isopropyl ether (100 mL). After filtration, the solid was washed with isopropyl ether (250 mL) twice, and the crude peptide was dried under reduced pressure for 2 h to afford intermediate 88 (1.0 g, crude) as a white solid.

To a mixture of intermediate 88 (1.0 g, crude) in MeCN/H₂O (5/5, v/v, 200 mL) was added NaHCO₃ to pH=8, then the mixture was stirred at 20° C. for 5 h. After filtration, the filtrate was purified by prep-HPLC (A: 0.075% TFA/H₂O, B: MeCN) directly, followed by lyophilization to afford Compound 251 (Strata 1.5-Biotin) (13.7 mg, 90.1% purity, 1.36% yield) as a white solid. LCMS: RT=1.00 min, MS cal.: 5001.93, mass observed: [M+3H]³⁺=1668.00, [M+4H]⁴⁺+=1251.20, [M+5H]⁵⁺=1001.10, [M+6H]⁶⁺=833.70.

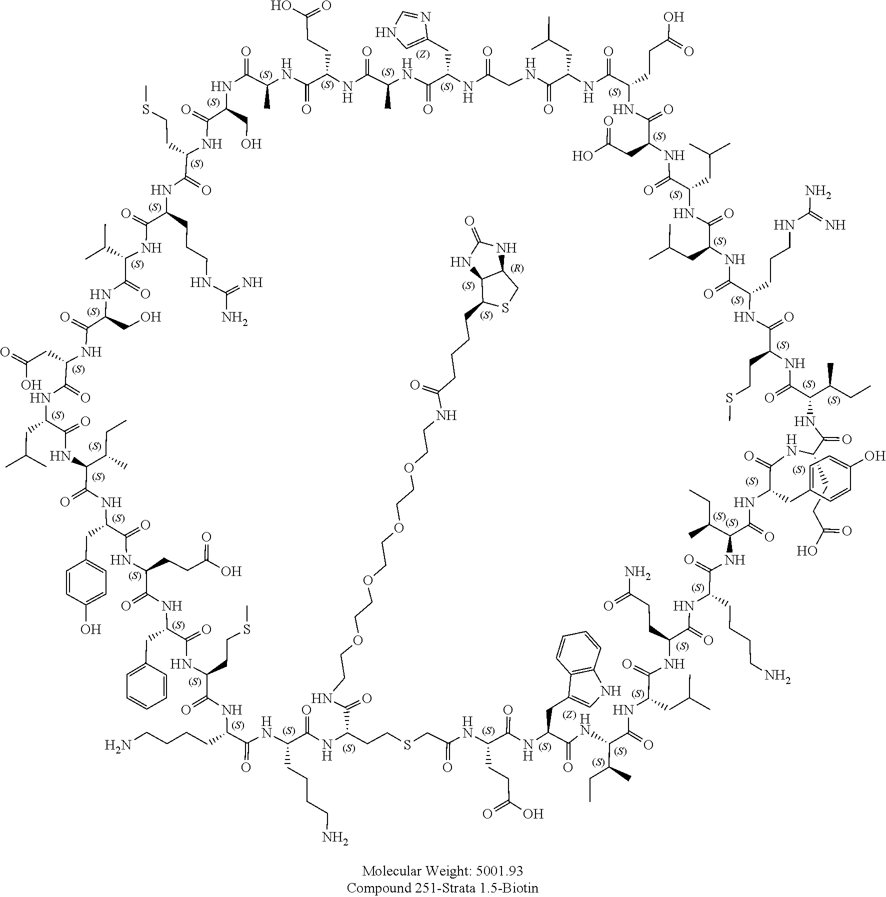

Compound 252 (Strata 1.5-Biotin) was synthesized using the same method as Compound 251. 0.20 mmol resin afforded Compound 252 (10.6 mg, 94.7% purity, 1.03% yield) as a white solid. LCMS: RT=1.00 min, MS cal.: 5129.09, mass observed: [M+3H]³⁺=1710.90, [M+4H]⁴⁺=1283.30, [M+5H]⁵⁺=1026.80, [M+6H]⁶⁺=855.70.

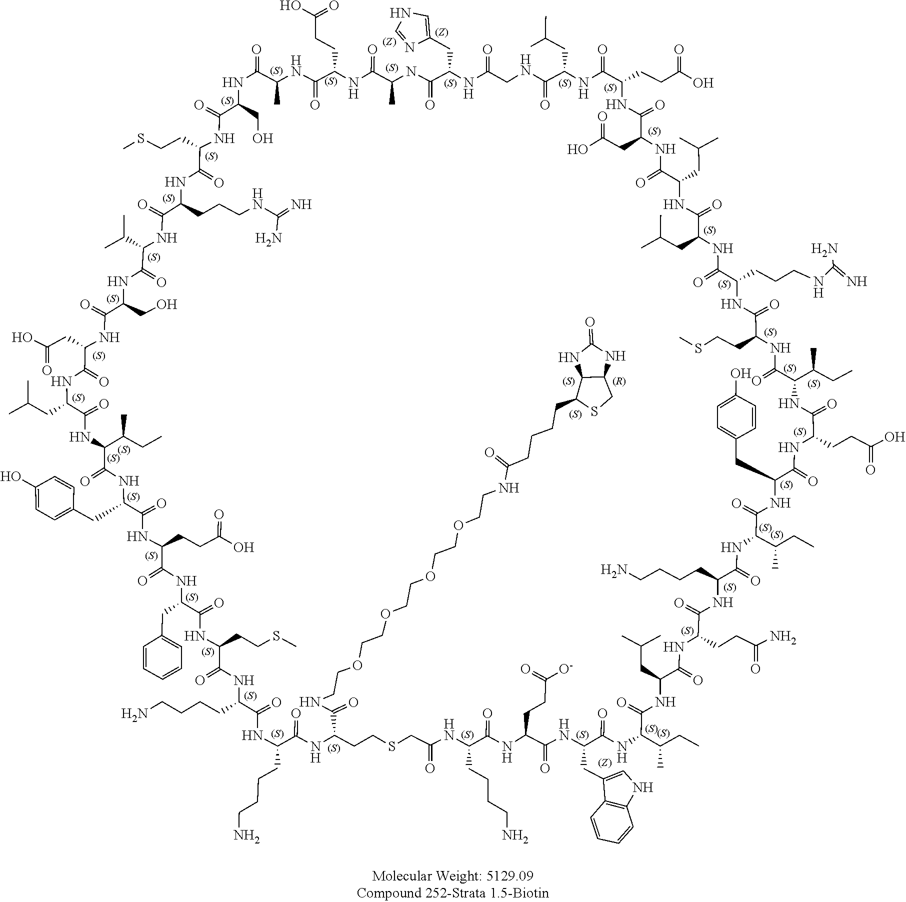

### Example 26. Procedure for Preparation of Compound 253 Strata 1.5-BCN

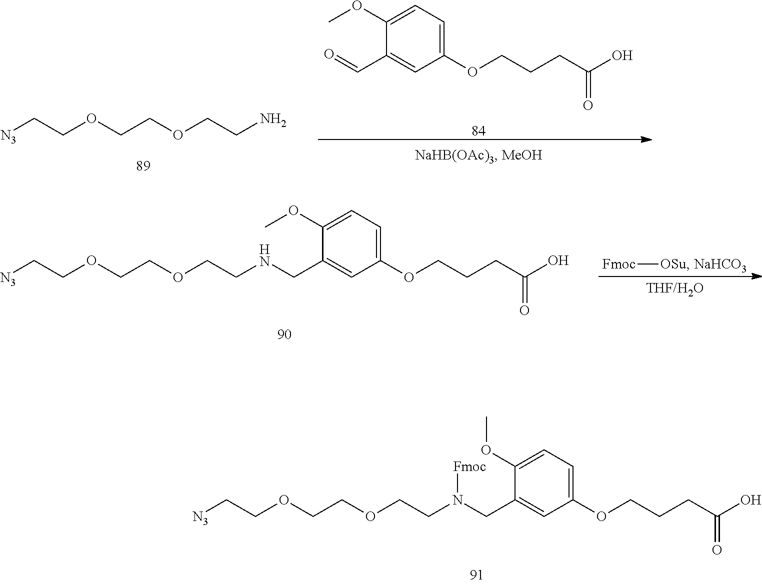

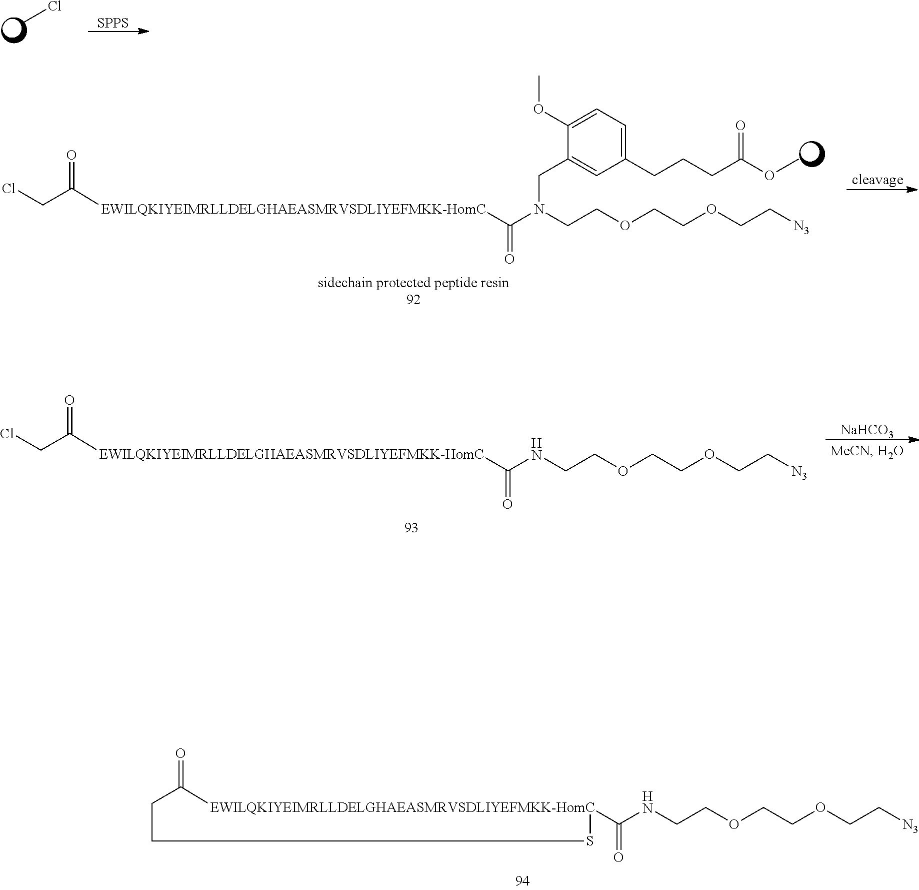

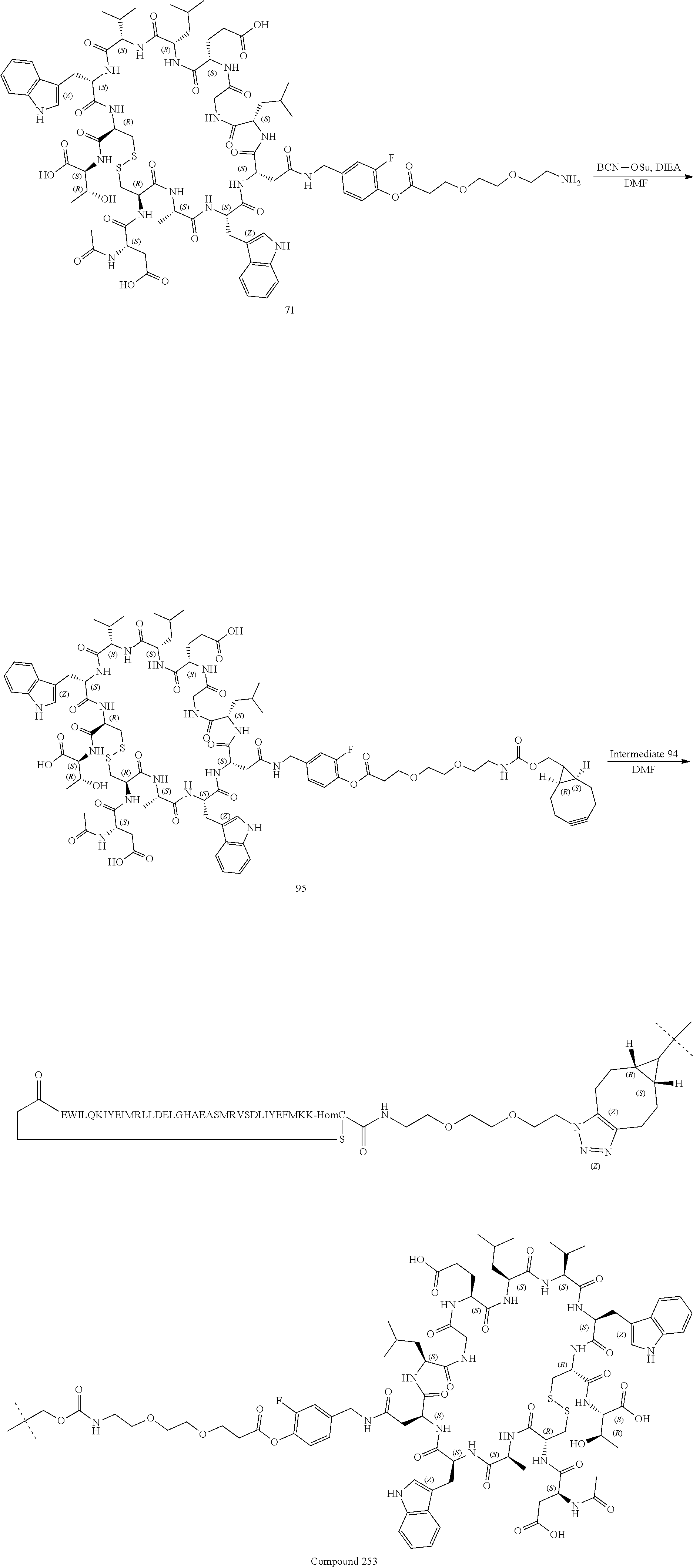

### Preparation of Intermediate 46

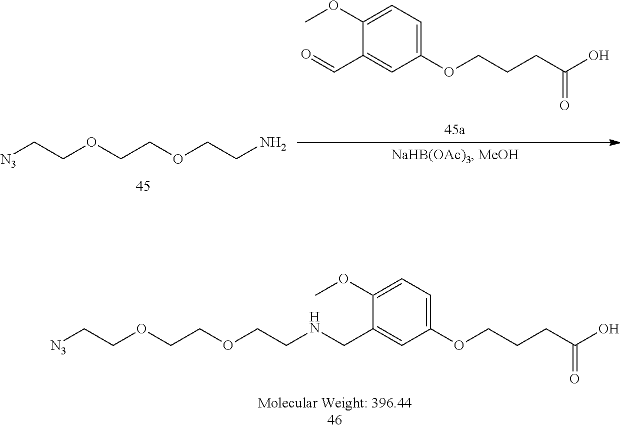 [SMILES: COc1ccc(OCCCC(=O)O)cc1C=O ; [N-]=[N+]=NCCOCCOCCN ; COc1ccc(OCCCC(=O)O)cc1CNCCOCCOCCN=[N+]=[N-]]

To a solution of intermediate 45a (1.64 g, 6.89 mmol, 1.20 equiv.), intermediate 45 (1.00 g, 5.74 mmol, 1.00 equiv.) in MeOH (10 mL) was added NaHB(OAc)₃ (1.82 g, 8.61 mmol, 1.50 equiv.) slowly at 0° C. Then the reaction mixture was stirred at 20° C. for 2 h. The reaction was quenched with H₂O (10 mL). The mixture was purified by prep-HPLC (A: 0.075% TFA/H₂O, B: MeCN) directly, then lyophilized to afford intermediate 46 (2.00 g, 80% purity, 87.9% yield) as a white solid. LCMS: RT=0.775 min, MS cal.: 396.44, mass observed: [M+H]⁺=397.0.

### Preparation of Intermediate 47.

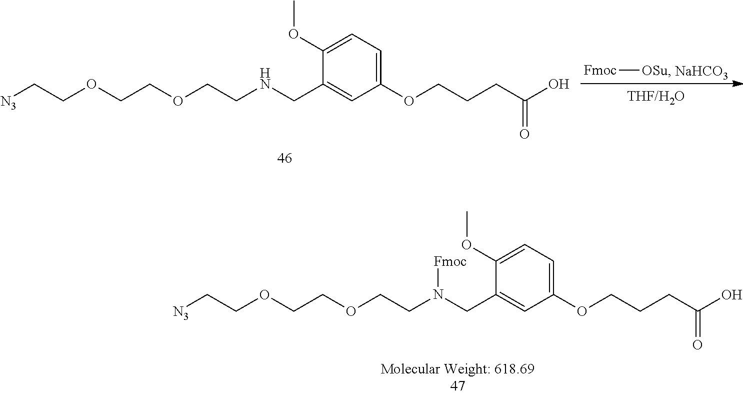

A mixture of intermediate 46 (2.00 g, 5.04 mmol, 1.00 equiv.) and Fmoc-OSu (2.55 g, 7.57 mmol, 1.50 equiv.) was dissolved in THF (20 mL) and H₂O (10 mL). Then NaHCO₃ (423.8 mg, 5.04 mmol, 196.2 uL, 1.00 equiv.) was added and the mixture was stirred at 20° C. for 2 h. The mixture was acidified by 1 M HCl to pH=6, purified by prep-HPLC (A: 0.075% TFA/H₂O, B: MeCN) directly, then lyophilized to afford intermediate 47 (1.83 g, 95.5% purity, 58.6% yield) as a white solid. LCMS: RT=1.017 min, MS cal.: 618.68, mass observed: [M+H]⁺=619.2

### Preparation of Intermediate 50

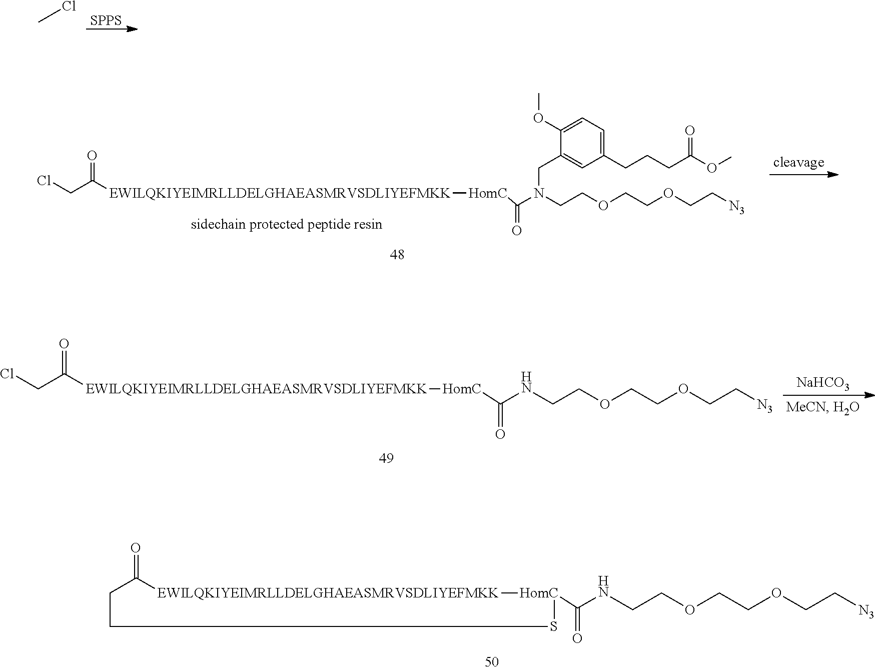

Intermediate 50 was synthesized using the same method as Compound 251, which was performed by following the procedure mentioned in [0157] and [0163](Page 60-63).

0.20 mmol resin afforded intermediate 50 (32.0 mg, 3.39% yield) as a white solid. LCMS: RT=1.05 min, MS cal.: 4713.52, mass observed: [M+3H]³⁺=1572.00, [M+4H]⁴⁺=1179.31.

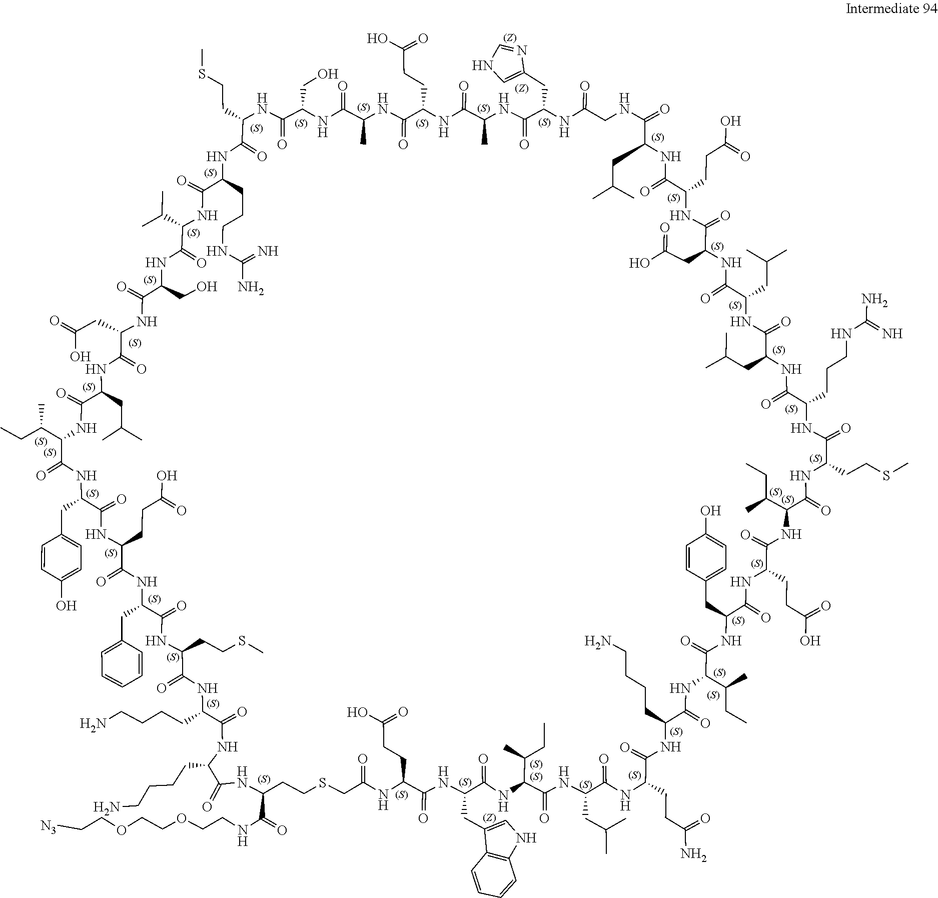

### Preparation of Intermediate 95

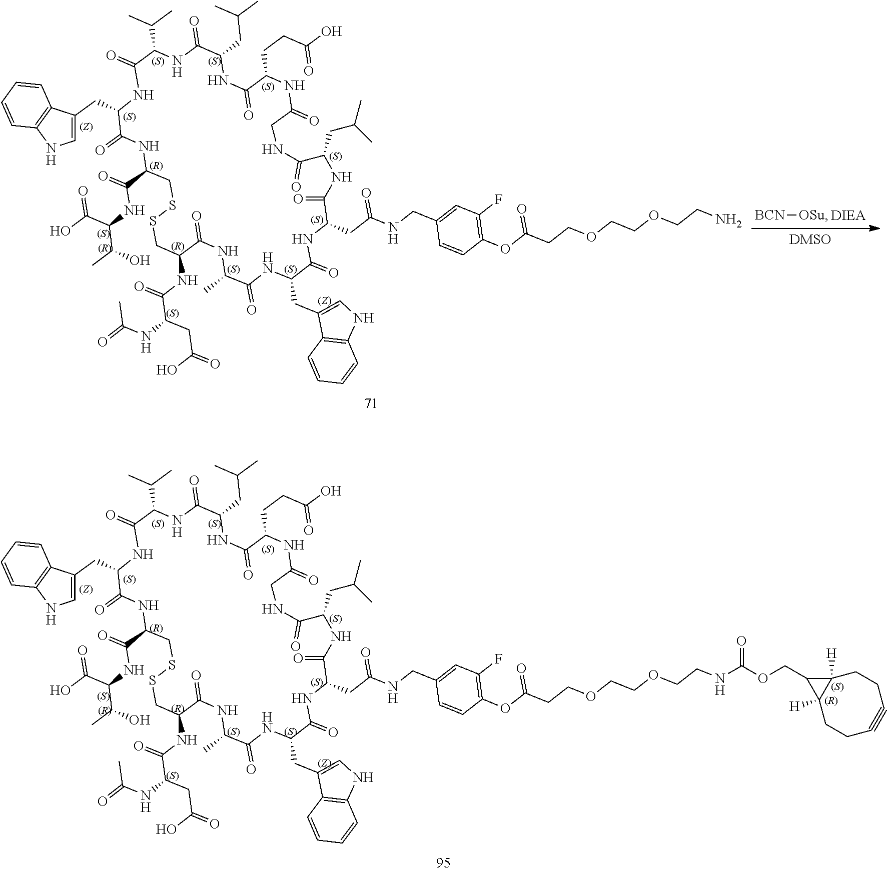

A mixture of intermediate 71 (2.00 g, 1.09 mmol, 1.00 equiv.), BCN-OSu (476.75 mg, 1.64 mmol, 1.50 equiv.), DIEA (564.0 mg, 4.36 mmol, 760.19 μL, 4.00 equiv.) in DMSO (30 mL) was stirred at 15° C. for 1 h. The mixture was purified by prep-HPLC (A: 0.075% TFA/H2O, B: MeCN) directly to afford intermediate 95 (2.20 g, 85% purity 42.65% yield) as a white solid. LCMS: RT=1.15 min, MS cal.: 2009.23, mass observed: [M+2H]2+=1004.86, [M-44+2H]2+=982.81.

### Preparation of Compound 253 (251-Strata 1.5-BCN)

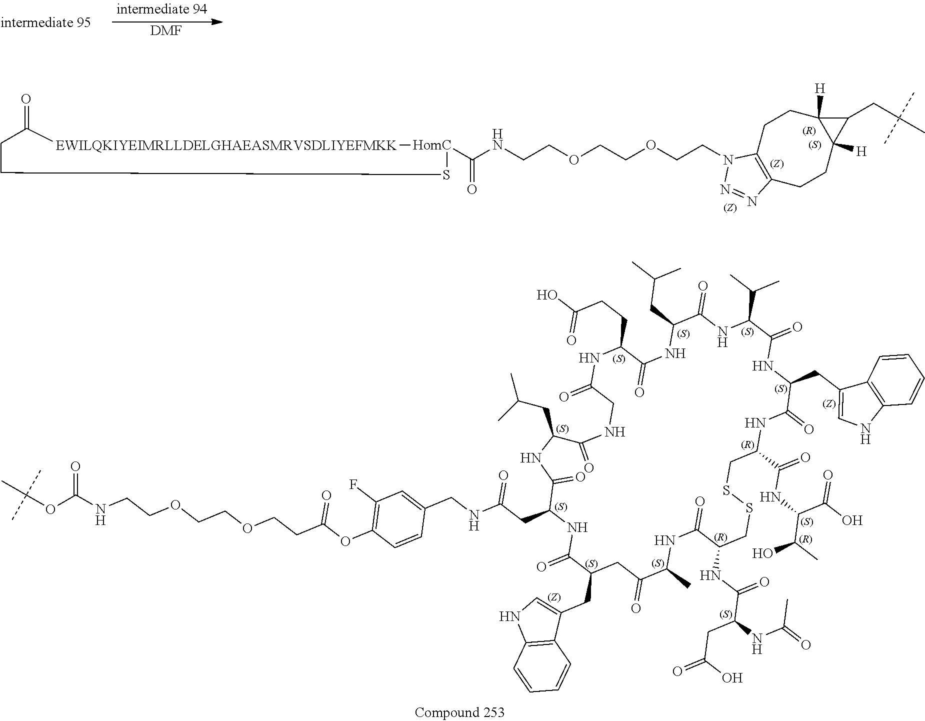

A mixture of intermediate 95 (6.82 mg, 3.39 μmol, 1.00 equiv.), intermediate 94 (16 mg, 3.39 μmol, 1.00 equiv.) in DMF (2.0 mL) was stirred at 20° C. for 5 h. The mixture was purified by prep-HPLC (A: 0.075% TFA/H₂O, B: MeCN) directly to afford Compound 253 (2.8 mg, 77.8% purity, 11.83% yield) as a white solid. LCMS: RT=1.10 min, MS cal.: 6722.75, mass observed: [M+4H]⁴⁺+=1682.11, [M-44+2H]²⁺=982.81.

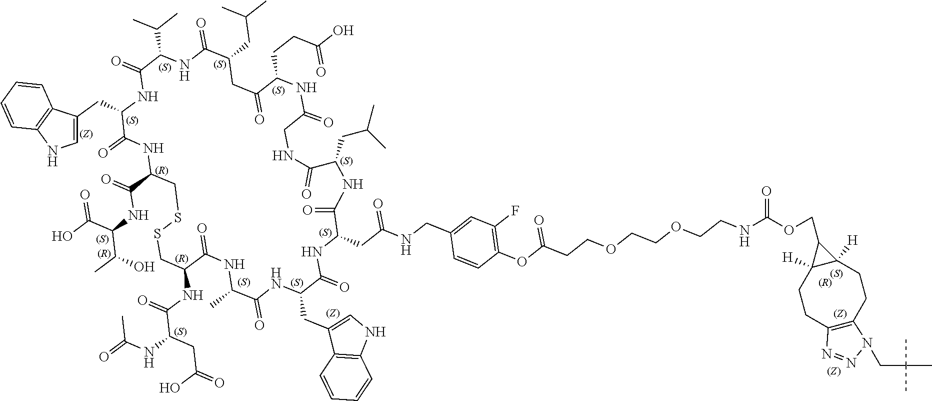

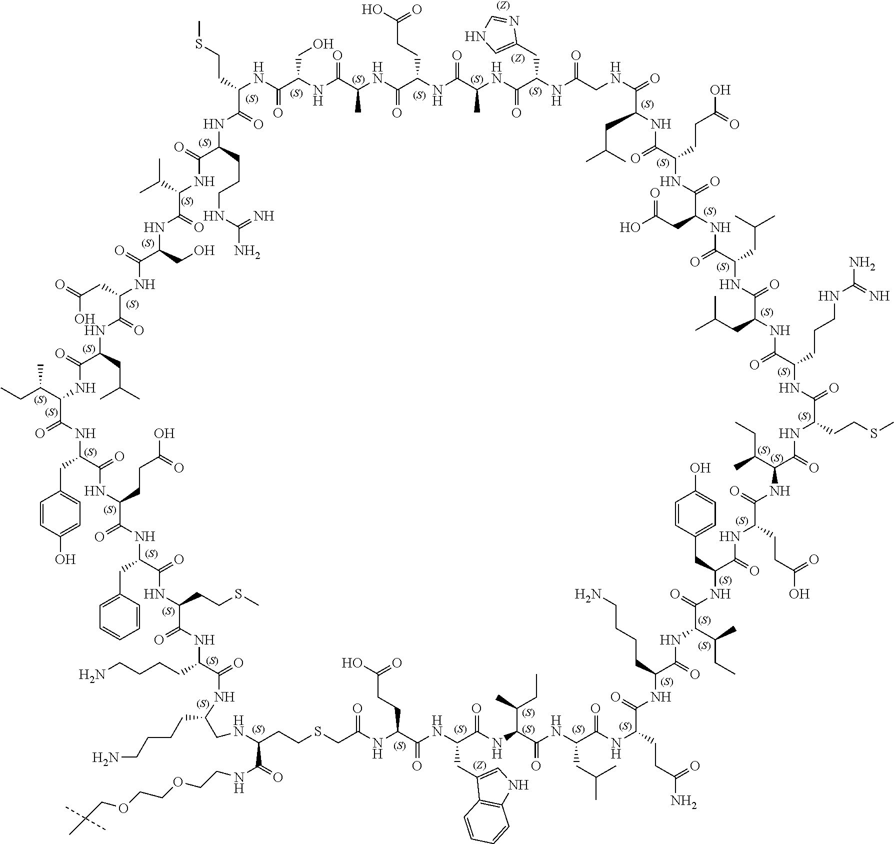

### Example 27. Examination of the Ternary Binding of SARS-CoV-2 Spike Protein

293T cells stably expressing SARS-CoV-2 Spike-GFP protein on their surface were incubated with a concentration range of compound for 30 minutes at 37° C.-5% CO₂ in Roswell Park Memorial Institute Medium (RPMI)-5% low IgG serum with 10 μg/ml IgG-Alexa Fluor® 647 (AF647). The cells were washed twice using the same medium, resuspended in phosphate-buffered saline (PBS)-1% bovine serum albumin (BSA), and acquired on Attune™ NxT Flow Cytometer. Ternary binding data of SARS-CoV-2 Spike Protein is shown in Table 21. In some embodiments, mean fluorescence intensity of AF647 as well as 00 AF647 positive in GFP positive gate are reported as readouts in this assay.

TABLE 21
SARS-CoV-2 Spike Protein Ternary Binding Assay Data

| | | | SARS-CoV-2 |
|---|---|---|---|
| | | | Ternary binding |
| Number | Compound No. | Synonyms | EC₅₀ (nM) |
| 1 | 168 | Compound | 0.19 |
| | | 1477-56-18 - | |
| | | (Peg8)3-uABT | |
| 2 | 167 | Compound | 0.24 |
| | | 1476-56-18 - | |
| | | (PAS)4-uABT | |
| 3 | 166 | Compound | 0.23 |
| | | 1475-56-18 - | |
| | | G8-uABT | |
| 4 | 165 | Compound | 0.11 |
| | | 1474-56-1 - | |
| | | (PAS)4-uABT | |
| 5 | 174 | Compound | 0.14 |
| | | 1461 | |
| 6 | 173 | Compound | 0.04 |
| | | 1460 | |
| 7 | 172 | Compound | 0.02 |
| | | 1459 | |
| 8 | 171 | Compound | 0.01 |
| | | 1458 | |
| 9 | 170 | Compound | 0.02 |
| | | 1457 | |
| 10 | 169 | Compound | 0.01 |
| | | 1456 | |
| 11 | 147 | Compound | 0.02 |
| | | 1455 | |
| 12 | 146 | Compound | 0.02 |
| | | 1454 | |
| 13 | 145 | Compound | 0.04 |
| | | 1453 | |
| 14 | 144 | Compound | 0.03 |
| | | 1452 | |
| 15 | 143 | Compound | 0.05 |
| | | 1451 | |
| 16 | 142 | Compound | 0.05 |
| | | 1450 | |
| 17 | 141 | Compound | 3.4 |
| | | 1335- | 2.1 |
| | | DHAM(52-18)- | |
| | | uABT | |
| 18 | 140 | Compound | >100.00 |
| | | 1334- | |
| | | DHAM(50-18)- | |
| | | uABT | |
| 19 | 139 | Compound | 17 |
| | | 1326- | 13 |
| | | DHAM(52-18)- | |
| | | uABT | |
| 20 | 164 | Compound | >100.00 |
| | | 1325- | |
| | | DHAM(50-18)- | |
| | | uABT | |
| 21 | 116 | Compound | 9.3 |
| | | 1253- | 11 |
| | | DHAM_batch2 | |
| 22 | 112 | Compound | 0.45 |
| | | 1249-DHAM- | 0.55 |
| | | Batch 1 | |
| 23 | 122 | Compound | >10.00 |
| | | 1264_batch1, | >10.00 |
| | | Compound | |
| | | 1264_batch2 | |
| 24 | NA | Compound | 0.56 |
| | | 1263, | 0.58 |
| | | Compound | 0.32 |
| | | 1263-#809 | 0.33 |
| 25 | 121 | Compound | 0.83 |
| | | 1262_batch1, | 0.78 |
| | | Compound | |
| | | 1262_batch2 | |
| 26 | 121 | Compound | |
| | | 1262_batch1, | |
| | | Compound | |
| | | 1262_batch2 | |
| 27 | 130 | Compound | 0.11 |
| | | 1261 | 0.1 |
| 28 | 130 | Compound | |
| | | 1261 | |
| 29 | 120 | Compound | 0.33 |
| | | 1260_batch1, | 0.31 |
| | | compound | |
| | | 1260 | |
| 30 | 118 | Compound | 0.53 |
| | | 1255- | |
| | | DHAM_batch 1 | 0.61 |
| 31 | 115 | Compound | 0.34 |
| | | 1252-DHAM | 0.36 |
| | | | 0.23 |
| | | | 0.26 |
| 32 | 113 | Compound | 0.48 |
| | | 1250-DHAM | 0.47 |
| 33 | 111 | Compound | 0.1 |
| | | 1248-DHAM | >10.00 |
| | | | 0.09 |
| | | | >10.00 |
| | | | 0.02 |
| | | | 0.01 |
| 34 | 110 | Compound | 0.14 |
| | | 1247- | 0.13 |
| | | DHAM_Batch 1 | |
| 35 | 119 | Compound | 2.4 |
| | | 1256-DHAM | 2.9 |
| 36 | 117 | Compound | 4.9 |
| | | 1254-DHAM | 4.4 |
| 37 | 108 | Compound | 2.7 |
| | | 1197- | |
| | | DHAM(19-56)- | 1.9 |
| | | GQAP-uABT | |
| 38 | 107 | Compound | 3.1 |
| | | 1196- | |
| | | DHAM(19-56)- | 4.5 |
| | | G4-uABT | |
| 39 | 106 | Compound | 2.6 |
| | | 1195- | |
| | | DHAM(19-56)- | 2.5 |
| | | G4-uABT | |
| 40 | NO NUMBER | Compound | >100.00 |
| | BUT | 1144-N1- | >100.00 |
| | STRUCTURE IN | DHAM(18-56) | |
| | SHEET | | |
| 41 | 109 | Compound | 1.2 |
| | | 1198- | |
| | | DHAM(19-56)- | 2.2 |
| | | GQAP-uABT | |
| 42 | 128 | Compound | 0.13 |
| | | 1168-NEW | |
| | | LCB1 | 0.15 |
| 43 | 159 | Compound | 3 |
| | | 1172-N1- | |
| | | DHAM(18-56) | 2.8 |
| 44 | 161 | Compound | 2.2 |
| | | 1171-N1- | |
| | | DHAM(18-56)- | 2.6 |
| | | btch2 | |
| 45 | 160 | Compound | 1.4 |
| | | 1170-N1- | |
| | | DHAM(18-56)- | 2.2 |
| | | batch2 | |
| 46 | 129 | Compound | 2.3 |
| | | 1135-N1- | |
| | | DHAM(18-56) | 3 |
| 47 | NO | KPMW357-5 | 0.16 |
| | STRUCTURE OR | | 0.31 |
| | COMP NO IN | | 0.03 |
| | TABLE | | 0.29 |
| | | | 0 |
| | | | 0 |
| | | | 0 |
| | | | 0 |
| | | | 0 |
| | | | 0 |
| | | | 0 |
| 48 | 208 | Compound | 0 |
| | | 1193-LCB1 | 0.06 |
| 49 | 163 | Compound | 0.14 |
| | | 1174-N1- | 0.43 |
| | | DHAM(18-56) | 0.67 |
| | | | 2 |
| 50 | 162 | Compound | 0.02 |
| | | 1173-N1- | |
| | | DHAM(18- | 0.11 |
| | | 56)_Batch1, | |
| | | Compound | |
| | | 1173-N1- | 0.38 |
| | | DHAM(18- | 1.2 |
| | | 56)_Batch2 | |
| 51 | 156 | Compound | >100.00 |
| | | 1153-N1- | |
| | | DHAM(18-48) | |
| 52 | 155 | Compound | 0.02 |
| | | 1152-N1- | 1.2 |
| | | DHAM(18-52) | 8.9 |
| 53 | 154 | Compound | >100.00 |
| | | 1151-N1- | |
| | | DHAM(23- | |
| | | 56)_Batch1, | |
| | | Compound | |
| | | 1151-N1- | |
| | | DHAM(23- | |
| | | 56)_Batch2 | |
| 54 | 153 | Compound | >10.00 |
| | | 1150-N1- | >10.00 |
| | | DHAM(28-56) | >10.00 |
| | | | >10.00 |
| 55 | 152 | Compound | >100.00 |
| | | 1149-N1- | |
| | | DHAM(32-56) | |
| 56 | 151 | Compound | >10.00 |
| | | 1148-N1- | |
| | | DHAM(37-56) | |
| 57 | 150 | Compound | 0.07 |
| | | 1147-N1- | 0.06 |
| | | DHAM(18-57) | 0.46 |
| | | | 1.2 |
| 58 | 148 | Compound | 0.06 |
| | | 1134-N1- | 0.08 |
| | | DHAM(18-56) | 0.34 |
| | | | 1.2 |
| 59 | 149 | Compound | 0.09 |
| | | 1133-N1- | 0.28 |
| | | DHAM(18-56) | 0.88 |
| | | | 2.2 |
| 60 | 187 | Compound | 0 |
| | | 981-Strata 1.5 | 0.07 |
| 61 | 158 | Compound | 0.61 |
| | | 1169-NEW | 9.9 |
| | | LCB1 | 6.8 |
| 62 | 193 | Compound | 2.3 |
| | | 1085- | 0.24 |
| | | LCB1TurnMut | |
| 63 | 222 | Compound | 0.02 |
| | | 1081- | 0.02 |
| | | LCB1TurnMut | |
| 64 | 125 | Compound | 0.25 |
| | | 1043- | |
| | | N3_batch2 | |
| 65 | 218 | Compound | 0.1 |
| | | 1065-(Biot- | |
| | | 2435-5) | |
| 66 | 216 | Compound | 0.46 |
| | | 1057-(Biot- | |
| | | 2435-3) | |
| 67 | STRUCTURE IN | Compound | 0 |
| | TABLE BUT NO | 993-Strata 1.5- | 0.08 |
| | NUMBER IN | Biotin | |
| | TABLE | | |
| 68 | NOT IN TABLE | Compound | 0.03 |
| | | 992-Strata 1.5- | 0.05 |
| | | Biotin | |
| 69 | 126 | Compound | 0.12 |
| | | 1044-N3, | 0.11 |
| | | Compound | 0.25 |
| | | 1044- | 0.79 |
| | | N3_batch2 | 0.24 |
| 70 | 124 | | 0.03 |
| | | | 0.02 |
| | | | 0.08 |
| | | | 0.53 |
| | | | 0.84 |
| | | Compound | 0.62 |
| | | 1042-N1, | |
| | | Compound | 0.89 |
| | | 1042- | |
| | | Nl_batch2, | 0.63 |
| | | Compound | 0.23 |
| | | 1042_batch3 | 0.23 |
| | | | 0.09 |
| | | | 0.95 |
| | | | 0.65 |
| | | | 0.07 |
| 71 | 123 | Compound | 0.05 |
| | | 1041-N1, | |
| | | Compound | 0.01 |
| | | 1041-N1_Batch | |
| | | 3, Compound | 0.18 |
| | | 1041- | |
| | | N2_batch2_2, | |
| | | Compound | |
| | | 1041_batch3 | |
| 72 | 209 | Compound | 1.1 |
| | | 964-12 | |
| | | families-10 | |
| 73 | 103 | Compound | >1.00 |
| | | 1140-N1- | >10.00 |
| | | DHAM(18-56), | |
| | | Compound | |
| | | 963-12 | |
| | | families-10 | |
| 74 | 181 | Compound | 0.77 |
| | | 920-cyclic | <0.30 |
| | | peptides | |
| 75 | 185 | Compound | 8.4 |
| | | 851-LCB3- | |
| | | NH2 | |
| 76 | 183 | Compound | 34 |
| | | 846-LCB1- | |
| | | NH2 | |

### Example 28. Examination of the Neutralization of Live SARS-CoV-2 Virus

Compounds and conjugated antibodies (human polyclonal antibody IgG 1, 2, 4, IVIG) were tested against wildtype (or variant) live SARS-CoV-2 Virus in triplicate. Virus was obtained from IIT Research Institute (IITRI, Chicago, IL) and Viroclinics (Rotterdam, Netherlands). Compounds and conjugated antibodies were serially diluted and incubated with a standardized virus concentration of 0.001 median tissue culture infectious dose (TCID₅₀)/cell at 37° C.±2° C. with 5.0%+/−1% CO₂ for 60 to 90 minutes. One-hundred microliters of the mixture was then transferred into the wells of a 96-well enzyme-linked immunosorbent assay (ELISA) plate which contained a monolayer of Vero E6 cells, and incubated in a humidified chamber at 37° C.±2° C. with 500±2% CO₂ for 60 to 90 minutes. Following the incubation, mixture was removed, the cells were washed, and 0.2 mL Dulbecco's Modified Eagle Medium (DMEM)-2% fetal bovine syndrome (FBS) (DMEM2) with compounds was added to each well. The cells were then incubated in a humidified chamber at 37° C.±2° C. with 5±100 CO₂. After 48±6 hours, the cells were fixed with paraformaldehyde and stained by anti-SARS-2 nucleoprotein monoclonal antibody (Sino Biological, Wayne, PA) followed by peroxidase-conjugated goat anti-mouse IgG (SeraCare®, Milford, MA). The wells were developed using 3,3′,′5,5′-tetramethylbenzidine (TMB) substrate solution. The reaction was stopped by acidification. The ELISA plates were read at 450 nm on a spectrophotometer ELISA plate reader.

For each well, the percent neutralization of virus was calculated as the percentage of reduction of the absorbance value with respect to the virus control by the following formula:

$$percent inhibition = 100 - [ ( A 450 of antibody dilution - A 450 of cell control ) / ( A 450 of virus control - A 450 of cell control ) ] \times 100.$$

The neutralization data for live SARS-CoV-2 virus is shown in Table 22.

TABLE 22
SARS-CoV-2 Live Virus Neutralization Assay Data

| | | | SARS-CoV-2 Viral Variant/Isolate Examined | | | | |
|---|---|---|---|---|---|---|---|
| | | | | | | Omicron | |
| | | | Seattle WA | Gamma | Delta | BA.1 | Delta |
| | | | (IITRI) | (IITRI) | (IITRI) | (Viroclinics) | (Viroclinics) |
| | Cmp. | | EC₅₀ | EC₅₀ | EC₅₀ | IC₅₀ | IC₅₀ |
| Number | No. | Synonyms | (nM) | (nM) | (nM) | (nM) | (nM) |
| 1 | 168 | Compound | | | 30.2 | | |
| | | 1477-56-18 - | | | | | |
| | | (Peg8)3- | | | | | |
| | | uABT | | | | | |
| 2 | 167 | Compound | | | 1.85 | | |
| | | 1476-56-18 - | | | | | |
| | | (PAS)4- | | | | | |
| | | uABT | | | | | |
| 3 | 166 | Compound | | >1000.00 | 1.42 | | |
| | | 1475-56-18 - | | | | | |
| | | G8-uABT | | | | | |
| 4 | 165 | Compound | 0.029 | | 0.02 | | |
| | | 1474-56-1 - | | | | | |
| | | (PAS)4- | | | | | |
| | | uABT | | | | | |
| 5 | 174 | Compound | | 0.11 | >1000.00 | 0.01 | |
| | | 1461 | | | | | |
| 6 | 173 | Compound | | 0.071 | 130.84 | 0.11 | |
| | | 1460 | | | | | |
| 7 | 172 | Compound | | <100E−06 | 1.02 | 0.12 | |
| | | 1459 | | | | | |
| 8 | 171 | Compound | | <1.0E−03 | 0 | 0.03 | |
| | | 1458 | | | | | |
| 9 | 170 | Compound | | 0.0058 | >1000.00 | 0.01 | |
| | | 1457 | | | | | |
| 10 | 169 | Compound | | 0.0018 | >1000.00 | 0.04 | |
| | | 1456 | | | | | |
| 11 | 147 | Compound | | 0.00073 | 670.11 | <0.00 | |
| | | 1455 | | | | | |
| 12 | 146 | Compound | | <100E−06 | 206.07 | 0 | |
| | | 1454 | | | | | |
| 13 | 145 | Compound | | 0.0031 | >1000.00 | 0.01 | |
| | | 1453 | | | | | |
| 14 | 144 | Compound | | <100E−06 | 122.06 | 0 | |
| | | 1452 | | | | | |
| 15 | 143 | Compound | | <100E−06 | >1000.00 | <0.00 | |
| | | 1451 | | | | | |
| 16 | 142 | Compound | | <100E−06 | >1000.00 | 0 | |
| | | 1450 | | | | | |
| 17 | 120 | Compound | 0.001 | >100 | | | |
| | | 1260_batch 1, | | | | | |
| | | compound | | | | | |
| | | 1260 | | | | | |
| 18 | 111 | Compound | 0.14 | >100 | <1.0E−03 | >1000.00 | 0.03 |
| | | 1248- | | | | | |
| | | DHAM | | | | | |
| 19 | 110 | Compound | 0.0058 | >100 | 0.0077 | >1000.00 | 0.01 |
| | | 1247- | | | | | |
| | | DHAM_Batch1 | | | | | |
| 20 | 108 | Compound | 1.4 | >1.0E+03 | | | |
| | | 1197- | | | | | |
| | | DHAM(19- | | | | | |
| | | 56) - | | | | | |
| | | GQAP- | | | | | |
| | | uABT | | | | | |
| 21 | 106 | Compound | 1.4 | >1.0E+03 | | | |
| | | 1195- | | | | | |
| | | DHAM(19- | | | | | |
| | | 56) -G4- | | | | | |
| | | uABT | | | | | |
| 22 | 109 | Compound | 0.75 | >1.0E+03 | | | |
| | | 1198- | 0.3 | | | | |
| | | DHAM(19- | | | | | |
| | | 56) - | | | | | |
| | | GQAP- | | | | | |
| | | uABT | | | | | |
| 23 | 128 | Compound | 0.042 | >10 | | | |
| | | 1168-NEW | | | | | |
| | | LCB1 | | | | | |
| 24 | 129 | Compound | 1.3 | | | | |
| | | 1135-N1- | | | | | |
| | | DHAM(18- | | | | | |
| | | 56) | | | | | |
| 28 | 255 | Compound | 0.32 | | | | |
| | | 1194- | | | | | |
| | | LCB1-2020, | | | | | |
| | | Compound | | | | | |
| | | 1194- | | | | | |
| | | LCB1-2021 | | | | | |
| 29 | 155 | Compound | 14 | | | | |
| | | 1152-N1- | 16 | | | | |
| | | DHAM(18- | | | | | |
| | | 52) | | | | | |
| 30 | 149 | Compound | 1.8 | | | | |
| | | 1133-N1- | | | | | |
| | | DHAM(18- | | | | | |
| | | 56) | | | | | |
| 31 | 232 | Compound | 9.5 | | | | |
| | | 1159-N1- | 21 | | | | |
| | | DHAM(18- | | | | | |
| | | 53) | | | | | |
| 32 | 226 | Compound | 0.29 | | | | |
| | | 1138-N1- | 1.3 | | | | |
| | | DHAM(18- | | | | | |
| | | 56) | | | | | |
| 33 | 125 | Compound | 2.3 | | 0.053 | | |
| | | 1043- | | | | | |
| | | N3_batch2 | | | | | |
| 34 | 206 | Compound | 0.19 | | | | |
| | | 1045- | | | | | |
| | | 859acid_batch1, | | | | | |
| | | Compound | | | | | |
| | | 1045- | | | | | |
| | | 859acid_batch2, | | | | | |
| | | Compound | | | | | |
| | | 1045- | | | | | |
| | | 859acid_batch3 | | | | | |
| 35 | 129 | Compound | 0.73 | | >100E−06 | | |
| | | 1044-N3, | 1.2 | | | | |
| | | Compound | 2.2 | | | | |
| | | 1044- | 0.03 | | | | |
| | | N3_batch2 | 0.029 | | | | |
| 36 | 124 | Compound | 0.023 | >100 | 0.00081 | >1000.00 | 0.01 |
| | | 1042-N1, | 0.69 | | | | |
| | | Compound | 0.82 | | | | |
| | | 1042- | 0.024 | | | | |
| | | N1_batch2, | 0.04 | | | | |
| | | Compound | 0.011 | | | | |
| | | 1042_batch | | | | | |
| | | 3 | | | | | |
| 37 | 103 | Compound | 0.13 | >100 | | | |
| | | 1140-N1- | | | | | |
| | | DHAM(18- | | | | | |
| | | 56), | | | | | |
| | | Compound | | | | | |
| | | 963-12 | | | | | |
| | | families-10 | | | | | |
| 38 | 137 | Compound | 0.31 | >1.0E+03 | <100E−06 | >1000.00 | 0.93 |
| | | 919-cyclic | 3.4 | | | | |
| | | peptides | | | | | |
| 39 | 136 | Compound | 0.084 | <0.00 | 0.86 | | |
| | | 918-cyclic | | | | | |
| | | peptides | | | | | |
| 40 | 176 | Compound | 7.7 | | | | |
| | | 874 | <5.0 | | | | |
| 41 | 175 | Compound | 21 | | | | |
| | | 872 | | | | | |
| 42 | 138 | Compound | 23 | >1.0E+03 | | | |
| | | 871 | | | | | |
| 43 | 185 | Compound | <0.60 | | | | |
| | | 851-LCB3- | <5.0 | | | | |
| | | NH2 | | | | | |
| 44 | 195 | Compound | | 0.0045 | | | |
| | | 850-LCB3- | | | | | |
| | | NH2 | | | | | |
| 45 | 184 | Compound | | 0.076 | | | |
| | | 849-LCB3- | | | | | |
| | | NH2 | | | | | |
| 46 | 183 | Compound | 0.15 | | | | |
| | | 846-LCB1- | <0.13 | | | | |
| | | NH2 | <5.0 | | | | |
| 47 | 105 | Compound | 0.057 | | | | |
| | | 845-LCB1- | | | | | |
| | | NH2 | | | | | |
| 48 | 104 | Compound | | 0.01 | | | |
| | | 840-LCB3 | | | | | |

### Example 30. Examination of the Neutralization of SARS-CoV-2 Spike Protein Pseudotyped Lentivirus

SARS-CoV-2 spike protein pseudotyped lentiviral stocks were produced by co-transfection of 293T cells with pMDL (lentiviral packaging plasmid), pLenti.GFp-NLuc (lentiviral GFP Luciferase plasmid), pcCoV2.S-Δ19 (or variant) (SARS-CoV2 Spike gene containing plasmid), and pRSV (lentiviral packaging plasmid) at a mass ratio of 4:3:4:1 by calcium phosphate coprecipitation. Virus-containing supernatant was harvested 2 days post-transfection, passed through a 0.45 m filter and concentrated by ultracentrifugation over a 20% sucrose cushion at 30,000 rotations per minute (RPM) for 90 minutes in an SW40.1 rotor in a Beckman Optima L-100K ultracentrifuge (Brea, CA). The pellet was resuspended to one-tenth of the initial volume in DMEM-10% FBS, aliquoted, and frozen at −80° C. Virus stocks were normalized by real-time PCR reverse transcriptase activity. To quantify the neutralizing activity, conjugates were serially diluted and incubated for 30 minutes at room temperature with pseudotyped virus, corresponding to approximately 2.5×10⁷ counts per second (cps) luciferase. The mixture was added to 1×10⁴ ACE2.293T cells, which corresponds to a multiplicity of invention (MOI) of 0.2, in a volume of 100 ul in a 96-well tissue culture plate. After 2 days, the medium was removed and Nano-Glo® Luciferase Assay Substrate (NanoLight™, Aurora, CO) was added to the wells. Luminescence was measured by an EnVision® 2103 Multilabel Microplate Reader (PerkinElmer®, Waltham, MA). The neutralization data for SARS-CoV-2 spike protein pseudotyped lentivirus is shown in Tables 23 and 24.

TABLE 23
SARS-CoV-2 Spike Protein Pseudotyped Lentivirus Neutralization Assay Data

| | | | SARS-CoV-2 Spike Protein in Pseudotyped Lentivirus Examined | | | | | | |
|---|---|---|---|---|---|---|---|---|---|
| | | | | Omicron | | | | | |
| | | | Omicron | BA.2 | N501Y | Mu | Kappa | Iota | Gamma |
| | Cpd. | | EC50 | EC50 | EC50 | EC50 | EC50 | EC50 | EC50 |
| No. | No. | Synonyms | (nM) | (nM) | (nM) | (nM) | (nM) | (nM) | (nM) |

| | | | | | | | |
|---|---|---|---|---|---|---|---|
| 1 | 122 | Compound | | | | | >1000.00 |
| | | 1264_batch1, | | | | | |
| | | Compound | | | | | |
| | | 1264_batch2 | | | | | |
| 2 | 121 | Compound | | | | | >1000.00 |
| | | 1262_batch1, | | | | | |
| | | Compound | | | | | |
| | | 1262_batch2 | | | | | |
| 3 | 120 | Compound | | | 0.028 | 0.029 | >1000.00 |
| | | 1260_batch1, | | | | | |
| | | compound | | | | | |
| | | 1260 | | | | | |
| 4 | 115 | Compound | | | | | >1000.00 |
| | | 1252-DHAM | | | | | |
| 5 | 111 | Compound | | >10 | 0.02 | 0.093 | >1000.00 |
| | | 1248-DHAM | | | | | >100.00 |
| 6 | 110 | Compound | | >10 | 0.52 | 0.12 | >1000.00 |
| | | 1247- | | | | | >1000.00 |
| | | DHAM_Batch1 | | | | | |
| 7 | 108 | Compound | | | | | >1000.00 |
| | | 1197- | | | | | |
| | | DHAM(19- | | | | | |
| | | 56) -GQAP- | | | | | |
| | | uABT | | | | | |
| 8 | 107 | Compound | | >1.0 | | | >1000.00 |
| | | 1196- | | | | | |
| | | DHAM(19- | | | | | |
| | | 56) -G4- | | | | | |
| | | uABT | | | | | |
| 9 | 106 | Compound | | | | | >1000.00 |
| | | 1195- | | | | | |
| | | DHAM(19- | | | | | |
| | | 56) -G4- | | | | | |
| | | uABT | | | | | |
| 10 | 109 | Compound | | >10 | | | >1000.00 |
| | | 1198- | | | | | |
| | | DHAM(19- | | | | | |
| | | 56) -GQAP- | | | | | |
| | | uABT | | | | | |
| 11 | 128 | Compound | | 2.5 | | | 123 |
| | | 1168-NEW | | | | | |
| | | LCB1 | | | | | |
| 12 | 129 | Compound | | >10 | | | >1000.00 |
| | | 1135-N1- | | | | | |
| | | DHAM(18- | | | | | |
| | | 56) | | | | | |
| 22 | 126 | Compound | 99 | | 0.26 | 0.27 | >1000.00 |
| | | 1044-N3, | | | 0.5 | 1.4 | >1000.00 |
| | | Compound | | | | | |
| | | 1044- | | | | | |
| | | N3_batch2 | | | | | |
| 23 | 124 | Compound | 1.9 | 8.9 | 0.13 | 0.087 | 189 |
| | | 1042-N1, | | | 0.097 | 0.15 | 548 |
| | | Compound | | | | | >200.00 |
| | | 1042- | | | | | |
| | | N1_batch2, | | | | | |
| | | Compound | | | | | |
| | | 1042_batch3 | | | | | |
| 24 | 123 | Compound | | 17 | 0.005 | 0.001 | 99 |
| | | 1041-N1, | | | | 8 | 32 |
| | | Compound | | | | | |
| | | 1041- | | | | | |
| | | N1_Batch_3, | | | | | |
| | | Compound | | | | | |
| | | 1041- | | | | | |
| | | N2_batch2_2, | | | | | |
| | | Compound | | | | | |
| | | 1041_batch3 | | | | | |
| 25 | 103 | Compound | | | | | >1000.00 |
| | | 1140-N1- | | | | | |
| | | DHAM(18- | | | | | |
| | | 56), | | | | | |
| | | Compound | | | | | |
| | | 963-12 | | | | | |
| | | families-10 | | | | | |
| 26 | 102 | Compound | | 0.17 | | | >1000.00 |
| | | 962-12 | | | | | |
| | | families-10 | | | | | |
| 27 | 137 | Compound | | 0.66 | | | >1000.00 |
| | | 919-cyclic | | | | | |
| | | peptides | | | | | |
| 28 | 136 | Compound | | >10 | 2.4 | 3 | >1000.00 |
| | | 918-cyclic | | | | | >1000.00 |
| | | peptides | | | | | |
| 29 | 138 | Compound | | >100 | 15 | 8.3 | >1000.00 |
| | | 871 | | | | | >1000.00 |
| 30 | 105 | Compound | 0.51 | | 0.7 | 0.41 | >1000.00 |
| | | 845-LCB1- | | | 0.29 | 0.053 | >10.00 |
| | | NH2 | | | 0.13 | 0.11 | >100.00 |
| | | | | | | | >200.00 |
| 31 | 104 | Compound | | | | | >1000.00 |
| | | 840-LCB3 | | | | | |

TABLE 24
Add'l SARS-CoV-2 Spike Protein Pseudotyped Lentivirus Neutralization Assay Data

| | | | SARS-CoV-2 Spike Protein in Pseudotyped Lentivirus Examined | | | | | | |
|---|---|---|---|---|---|---|---|---|---|
| | | | | | Delta | | | | |
| | | | Epsilon | E484K | plus | Delta | D614G | Beta | Alpha |
| | Cpd. | | EC50 | EC50 | EC50 | EC50 | EC50 | EC50 | EC50 |
| No. | No. | Synonyms | (nM) | (nM) | (nM) | (nM) | (nM) | (nM) | (nM) |
| 1 | 122 | Compound | | | 1.3 | | | | |
| | | 1264_batch1, | | | | | | | |
| | | Compound | | | | | | | |
| | | 1264_batch2 | | | | | | | |
| 2 | 121 | Compound | | | 0.23 | | | | |
| | | 1262_batch1, | | | | | | | |
| | | Compound | | | | | | | |
| | | 1262_batch2 | | | | | | | |
| 3 | 120 | Compound | | | 0.08 | 0.04 | >1.0E+03 | 0.085 | |
| | | 1260_batch1, | | | | | | | |
| | | compound 1260 | | | | | | | |
| 4 | 115 | Compound | | | | 0.8 | | | |
| | | 1252-DHAM | | | | | | | |
| 5 | 111 | Compound | | 1.9 | 0.51 | 0.94 | >1.0E+03 | 0.26 | |
| | | 1248-DHAM | | | 0.86 | 0.06 | | | |
| | | | | | | 0.19 | | | |
| 6 | 110 | Compound | | | 1.1 | 1.2 | >1.0E+03 | 0.58 | |
| | | 1247- | | | | 0.22 | | | |
| | | DHAM_Batch1 | | | | 0.45 | | | |
| 7 | 119 | Compound | | | | 42 | | | |
| | | 1256-DHAM | | | | | | | |
| 8 | 117 | Compound | | | | 74 | | | |
| | | 1254-DHAM | | | | | | | |
| 9 | 108 | Compound | | | | 0.13 | | | |
| | | 1197- | | | | 3.3 | | | |
| | | DHAM(19-56) - | | | | | | | |
| | | GQAP-uABT | | | | | | | |
| 10 | 107 | Compound | | | | 18 | | | |
| | | 1196- | | | | 0.4 | | | |
| | | DHAM(19-56) - | | | | 3 | | | |
| | | G4-uABT | | | | | | | |
| 11 | 106 | Compound | | | | 1.8 | | | |
| | | 1195- | | | | 0.02 | | | |
| | | DHAM(19-56)- | | | | | | | |
| | | G4-uABT | | | | | | | |
| 12 | 109 | Compound | | | | 2.5 | | | |
| | | 1198- | | | | 0.97 | | | |
| | | DHAM(19-56) - | | | | 0.02 | | | |
| | | GQAP-uABT | | | | | | | |
| 13 | 128 | Compound | | | | 0.33 | | | |
| | | 1168-NEW | | | | 0.07 | | | |
| | | LCB1 | | | | | | | |
| 14 | 129 | Compound | | | | 7.2 | | | |
| | | 1135-N1- | | | | 0.91 | | | |
| | | DHAM(18-56) | | | | 0.03 | | | |
| 15 | | KPMW357-5 | 0.47 | 0.17 | 0.21 | 0.08 | 0.03 | 0.11 | 0.34 |
| 16 | | KPMW357-5 | | | | | 0.02 | | |
| 17 | | KPMW357-5 | | | | | 0.04 | | |
| 18 | | KPMW357-5 | | | | | 0.04 | | |
| 19 | | KPMW357-5 | | | | | 0.04 | | |
| 20 | | KPMW357-5 | | | | | 0.04 | | |
| 21 | | KPMW357-5 | | | | | 0.07 | | |
| 22 | | KPMW357-5 | | | | | 0.06 | | |
| 23 | | KPMW357-5 | | | | | 0.06 | | |
| 24 | | KPMW357-5 | | | | | 0.04 | | |
| 25 | | KPMW357-5 | | | | | 0.01 | | |
| 26 | | KPMW357-5 | 0.023 | | | 0.05 | 0.04 | 0.15 | |
| 27 | | KPMW357-5 | | | | | 0.04 | | |
| 28 | | KPMW357-5 | | | | | 0.03 | | |
| 29 | | KPMW357-5 | | | | | 0.04 | | |
| 30 | | KPMW357-5 | | | | | 0.07 | | |
| 31 | | KPMW357-5 | | | | | 0.06 | | |
| 32 | | KPMW357-5 | | | | | 0.08 | | |
| 33 | | KPMW357-5 | | | | | 0.07 | | |
| 34 | | KPMW357-5 | | | | | 0.09 | | |
| 35 | | KPMW357-5 | | | | | 0.08 | | |
| 36 | | KPMW357-5 | 0.062 | | | 0.07 | 0.05 | 0.13 | 0.057 |
| 37 | | KPMW357-5 | 0.097 | | | 0.08 | 0.05 | 0.1 | 0.056 |
| 38 | | KPMW357-5 | | | | | 0.03 | | |
| 39 | | KPMW357-5 | | | | | 0.04 | | |
| 40 | | KPMW357-5 | | | | | 0.07 | | |
| 41 | | KPMW357-5 | | | | | 0.06 | | |
| 42 | | KPMW357-5 | | | | | 0.08 | | |
| 43 | | KPMW357-5 | | | | 0.23 | 0.07 | | |
| 44 | | KPMW357-5 | | | | 0.11 | 0.11 | | |
| 45 | | KPMW357-5 | | | | 0.31 | 0.06 | | |
| 46 | | KPMW357-5 | | | | 0.15 | 0.09 | | |
| 47 | | KPMW357-5 | | | | 0.41 | 0.04 | | |
| 48 | | KPMW357-5 | | | | | 0.09 | | |
| 49 | 155 | Compound | | | | 0.01 | | | |
| | | 1152-N1- | | | | | | | |
| | | DHAM(18-52) | | | | | | | |
| 50 | 232 | Compound | | | | 0.07 | | | |
| | | 1159-N1- | | | | | | | |
| | | DHAM(18-53) | | | | | | | |
| 51 | 226 | Compound | | | | 0.01 | | | |
| | | 1138-N1- | | | | | | | |
| | | DHAM(18-56) | | | | | | | |
| 52 | 126 | Compound | | 0.1 | 0.01 | 0.78 | 210 | | |
| | | 1044-N3, | | | 0.79 | 1.5 | | | |
| | | Compound | | | >1.00 | | | | |
| | | 1044- | | | >1.00 | | | | |
| | | N3_batch2 | | | >1.00 | | | | |
| 53 | 124 | Compound | 4.7 | 0.61 | 0.66 | 0.25 | 2.9 | | |
| | | 1042-N1, | | | | | | | |
| | | Compound | | | | | | | |
| | | 1042- | | | | | | | |
| | | N1_batch2, | | | | | | | |
| | | Compound | | | | | | | |
| | | 1042_batch3 | | | | | | | |
| 54 | 124 | Compound | 0.02 | 0.22 | | | | | |
| | | 1042-N1, | 0.01 | | | | | | |
| | | Compound | 0.21 | | | | | | |
| | | 1042- | 0.86 | | | | | | |
| | | N1_batch2, | 1.2 | | | | | | |
| | | Compound | >1.00 | | | | | | |
| | | 1042_batch3 | | | | | | | |
| 55 | 123 | Compound | 0 | 1.2 | 24 | 0.006 | | | |
| | | 1041-N1, | | | | 8 | | | |
| | | Compound | | | | | | | |
| | | 1041- | | | | | | | |
| | | N1_Batch3, | | | | | | | |
| | | Compound | | | | | | | |
| | | 1041- | | | | | | | |
| | | N2_batch2_2, | | | | | | | |
| | | Compound | | | | | | | |
| | | 1041_batch3 | | | | | | | |
| 56 | 103 | Compound | | 0.77 | | | | | |
| | | 1140-N1- | | | | | | | |
| | | DHAM(18-56), | | | | | | | |
| | | Compound 963- | | | | | | | |
| | | 12 families-10 | | | | | | | |
| 57 | 102 | Compound 962- | | 2.8 | 1.4 | 2.3 | | | |
| | | 12 families-10 | | | | 1.3 | | | |
| 58 | 137 | Compound 919- | | | | 1.7 | | | |
| | | cyclic peptides | | | | | | | |
| 59 | 137 | Compound 919- | | | | 0.37 | | | |
| | | cyclic peptides | | | | | | | |
| 60 | 137 | Compound 919- | | | | 0.07 | | | |
| | | cyclic peptides | | | | | | | |
| 61 | 136 | Compound 918- | | | 1.6 | 8.7 | >1.0E+03 | 5.9 | |
| | | cyclic peptides | | | | 1.1 | | | |
| | | | | | | 4.6 | | | |
| | | | | | | 0.08 | | | |
| 62 | 176 | Compound 874 | | | | 0.14 | | | |
| 63 | 175 | Compound 872 | | | | 1.1 | | | |
| 64 | 138 | Compound 871 | | >100 | 1.6 | 34 | >1.0E+03 | 6.6 | |
| | | | | | 5.9 | 1.5 | | | |
| | | | | | | 6.1 | | | |
| | | | | | | 1.4 | | | |
| 65 | 184 | Compound 849- | | | | <4.50 | | | |
| | | LCB3-NH2 | | | | | | | |
| 66 | 183 | Compound 846- | | | | <4.50 | | | |
| | | LCB1-NH2 | | | | | | | |
| 67 | 105 | Compound 845- | 0.063 | | 0.52 | 0.69 | 26 | 0.17 | |
| | | LCB1-NH2 | | | 0.16 | 0.27 | 1.5 | 0.57 | |
| | | | | | | 0.11 | 0.52 | | |
| | | | | | | >1.00 | | | |
| | | | | | | <4.50 | | | |
| 68 | 182 | Compound 844- | <4.50 | | | | | | |
| | | LCB1- | | | | | | | |
| | | NH2_Batch1, | | | | | | | |
| | | Compound 844- | | | | | | | |
| | | LCB1- | | | | | | | |
| | | NH2_Batch2 | | | | | | | |
| 69 | 104 | Compound 840- | | >1.0E+03 | 0.24 | >10.00 | | | |
| | | LCB3 | | | | <4.50 | | | |
| 70 | 225 | Compound 819- | | | <4.50 | | | | |
| | | LCB1NEW1, | | | | | | | |
| | | Compound | | | | | | | |
| | | 819_batch2 | | | | | | | |
| 71 | 205 | Compound 818- | | | | <4.50 | | | |
| | | LCB1NEW1 | | | | | | | |

### Example 31. Examination of the Inhibition of SARS-COV-2 Spike Protein Binding/Interaction with Angiotensin Converting Enzyme 2 (Competition Assay)

Inhibition of SARS-CoV-2 Spike protein binding with Angiotensin converting enzyme 2 (ACE2) was examined by electrochemiluminescence (ECL) detection competition assay using kits, reagents and instruments from Meso Scale Diagnostics, LLC (Rockville, MD). Measurements were made using V-PLEX® SARS-CoV-2 Panel 13 (ACE2) Kit and the V-PLEX SARS-CoV-2 Panel 23 (ACE2) Kit. The V-PLEX® SARS-CoV-2 Panel 13 (ACE2) Kit has Spike antigens from the following lineages: A (wild-type), (B.1.1.7), (B.1.351), (B.1.526.1), (B.1.617), (B.1.617.1), (B.1.617.2), (B.1.617.3), (P.1), and (P.2). The V-PLEX® SARS-CoV-2 Panel 23 (ACE2) Kit has Spike antigens from the following lineages: A (WT), (AY.3), (AY.4), (AY.4.2), (AY.5), (AY.6), (AY.7), (AY.12), (AY.14), (B.1.1.529; BA.1), (B.1.1.7), (B.1.351), (B.1.351.1), (B.1.617.2), (B.1.617.2+A144), and (P.1).

The assays were performed according to the protocols provided in the assay kit package inserts with the exception of using a 2× concentration of compound in diluent.

Briefly, 150 μL of Blocker A solution was added to each well of the provided plate, and the plate was incubated for 30 minutes at room temperature with shaking (700 RPM). After the incubation, each well was washed three times with 150 ul of 1×MSD Wash Buffer. Next, 25 ul/well of a 2× concentration of compound in diluent or 25 ul calibrator was combined with 25 ul of 1×SULFO-TAG Human ACE2 protein and incubated for 2 hours at room temperature with shaking (700 rpm). Each well was washed three times with 150 ul of 1×MSD Wash Buffer. Next, 150 ul of MSD FOLD Read Bugger B was added to each well. The plate was immediately read on the MSD® instrument.

Results are reported as the percent inhibition calculated using the following equation:

$$\% Inhibition = 1 - \frac{Average ECL Signal of ACE 2 Binding With Competitior Present}{Average ECL Signal of ACE 2 Binding With No Competitor} \times 100$$

IC₅₀ values are determined by fitting the results to a four-parameter logistic model, and the data is shown in Table 25.

TABLE 25
MSD ® ACE2 Competition Assay Data

| | | | SARS-CoV-2 Variant/Isolate Examined | | | | | | | | | |
|---|---|---|---|---|---|---|---|---|---|---|---|---|
| | | | | | | | India | India | | | | Wild |
| | | | Iota | Beta | Alpha | Gamma | (B.1.617) | (B.1.617.3) | Delta | Kappa | Zeta | Type |
| Num- | Cpd | | IC₅₀ | IC₅₀ | IC₅₀ | IC₅₀ | IC₅₀ | IC₅₀ | IC₅₀ | IC₅₀ | IC₅₀ | IC₅₀ |
| ber | No. | Synonyms | (nM) | (nM) | (nM) | (nM) | (nM) | (nM) | (nM) | (nM) | (nM) | (nM) |
| 1 | 168 | Compound | 4.71 | (IC50 | 6.63 | (IC50 | 4.28 | 3.88 | 3.54 | 3.41 | 3.32 | 3.26 |
| | | 1477- | | could | | could | | | | | | |
| | | 56-18- | | not be | | not be | | | | | | |
| | | (Peg8)3- | | calculated) | | calculated) | | | | | | |
| | | uABT | | | | | | | | | | |
| 2 | 167 | Compound | 15.84 | (IC50 | 18.71 | (IC50 | 14.23 | 12.2 | 11.72 | 11.14 | 9.83 | 10.76 |
| | | 1476- | | could | | could | | | | | | |
| | | 56-18- | | not be | | not be | | | | | | |
| | | (PAS)4- | | calculated) | | calculated) | | | | | | |
| | | uABT | | | | | | | | | | |
| 3 | 166 | Compound | 23.06 | (IC50 | 32.08 | (IC50 | 19.34 | 17.62 | 20.24 | 15.97 | 16.05 | 16.55 |
| | | 1475- | | could | | could | | | | | | |
| | | 56-18- | | not be | | not be | | | | | | |
| | | G8- | | calculated) | | calculated) | | | | | | |
| | | uABT | | | | | | | | | | |
| 4 | 165 | Compound | 0.89 | >100.00 | 1.17 | 60.33 | 1.24 | 1.02 | 0.68 | 0.74 | 0.79 | 0.83 |
| | | 1474- | | | | | | | | | | |
| | | 56-1- | | | | | | | | | | |
| | | (PAS)4- | | | | | | | | | | |
| | | uABT | | | | | | | | | | |
| 5 | 174 | Compound | 1.45 | >100.00 | 2.05 | 90.92 | 1.95 | 1.77 | 1.37 | 1.36 | 1.37 | 1.4 |
| | | 1461 | | | | | | | | | | |
| 6 | 173 | Compound | 1.5 | >100.00 | 2.06 | 92.28 | 1.74 | 1.55 | 1.33 | 1.23 | 1.24 | 1.21 |
| | | 1460 | | | | | | | | | | |
| 7 | 172 | Compound | 1.31 | >100.00 | 1.72 | >100.00 | 1.38 | 1.09 | 0.98 | 0.95 | 0.9 | 1.05 |
| | | 1459 | | | | | | | | | | |
| 8 | 171 | Compound | 1.48 | >100.00 | 1.86 | >100.00 | 1.81 | 1.46 | 1.3 | 1.13 | 1.13 | 1.25 |
| | | 1458 | | | | | | | | | | |
| 9 | 170 | Compound | 1.71 | >100.00 | 2.29 | >100.00 | 2.29 | 2.05 | 1.73 | 1.37 | 1.38 | 1.52 |
| | | 1457 | | | | | | | | | | |
| 10 | 169 | Compound | 1.43 | >100.00 | 1.78 | 83.15 | 1.86 | 1.72 | 1.3 | 1.17 | 1.19 | 1.22 |
| | | 1456 | | | | | | | | | | |
| 11 | 147 | Compound | 1.55 | >100.00 | 2.09 | 79.64 | 1.97 | 1.7 | 1.31 | 1.18 | 1.23 | 1.35 |
| | | 1455 | | | | | | | | | | |
| 12 | 146 | Compound | 1.22 | >100.00 | 1.58 | 64.32 | 1.48 | 1.33 | 1.07 | 1 | 0.99 | 1 |
| | | 1454 | | | | | | | | | | |
| 13 | 145 | Compound | 1.23 | >100.00 | 1.41 | 84.35 | 1.26 | 0.91 | 0.9 | 0.83 | 0.69 | 0.97 |
| | | 1453 | | | | | | | | | | |
| 14 | 144 | Compound | 1.45 | >100.00 | 1.84 | 91.9 | 1.74 | 1.38 | 1.33 | 1.19 | 1.02 | 1.17 |
| | | 1452 | | | | | | | | | | |
| 15 | 143 | Compound | 1.73 | >100.00 | 2.2 | 82.72 | 2.09 | 1.9 | 1.64 | 1.33 | 1.26 | 1.35 |
| | | 1451 | | | | | | | | | | |
| 16 | 142 | Compound | 1.55 | >100.00 | 1.98 | 88.15 | 1.87 | 1.67 | 1.29 | 1.25 | 1.19 | 1.35 |
| | | 1450 | | | | | | | | | | |
| 17 | NA | KPMW3 | 0.48 | 90.86 | 1.25 | 28.49 | 0.5 | 0.43 | 0.4 | 0.36 | 0.39 | 0.35 |
| | | 43-1 | | | | | | | | | | |
| 18 | 141 | Compound | 63.35 | (IC50 | 793.44 | (IC50 | 61.32 | 49.81 | 44.84 | 52.39 | 76.46 | 25.11 |
| | | 1335- | | could | (IC50 | could | 78.11 | 76.8 | 70.16 | 78.89 | 9.46 | 43.84 |
| | | DHAM | | not be | could | not be | 69.41 | 62.8 | 71.79 | 65.27 | 89.38 | 30.1 |
| | | (52-18)- | | calculated) | not be | calculated) | | | | | | |
| | | uABT | | (IC50 | calculated) | (IC50 | | | | | | |
| | | | | could | (IC50 | could | | | | | | |
| | | | | not be | could | not be | | | | | | |
| | | | | calculated) | not be | calculated) | | | | | | |
| | | | | (IC50 | calculated) | (IC50 | | | | | | |
| | | | | could | | could | | | | | | |
| | | | | not be | | not be | | | | | | |
| | | | | calculated) | | calculated) | | | | | | |
| 19 | 140 | Compound | 7155.01 | <10.00 | >10000.00 | <10.00 | 5471.88 | 4753.02 | 4787.38 | 4401.48 | 6044.15 | 1557.04 |
| | | 1334- | >1000.00 | (IC50 | <1.00 | <1.00 | >1000.00 | >1000.00 | >1000.00 | >1000.00 | >1000.00 | >1000.00 |
| | | DHAM | | could | | | | | | | | |
| | | (50-18)- | | not be | | | | | | | | |
| | | uABT | | calculated) | | | | | | | | |
| 20 | 139 | Compound | 1024.67 | (IC50 | 2375.33 | (IC50 | 1255.11 | 1133.1 | 617.39 | 1143.48 | 2551.46 | 315.31 |
| | | 1326- | >1000.00 | could | >1000.00 | could | >1000.00 | >1000.00 | 962.41 | >1000.00 | >1000.00 | 514.03 |
| | | DHAM | 841.29 | not be | >1000.00 | not be | 954.11 | 755.03 | 496.91 | 884 | >1000.00 | 258.8 |
| | | (52-18)- | | calculated) | | calculated) | | | | | | |
| | | uABT | | <1.00 | | <1.00 | | | | | | |
| | | | | (IC50 | | <1.00 | | | | | | |
| | | | | could | | | | | | | | |
| | | | | not be | | | | | | | | |
| | | | | calculated) | | | | | | | | |
| 21 | 164 | Compound | >10000.00 | (IC50 | >10000.00 | <10.00 | >10000.00 | >10000.00 | >10000.00 | >10000.00 | >10000.00 | >10000.00 |
| | | 1325- | | could | | | | | | | | |
| | | DHAM | | not be | | | | | | | | |
| | | (50-18)- | | calculated) | | | | | | | | |
| | | uABT | | | | | | | | | | |
| 22 | 135 | Compound | 27.94 | (IC50 | >100.00 | (IC50 | 30.13 | 23.28 | 29.01 | 27.05 | 41.38 | 12.23 |
| | | 1333- | | could | | could | | | | | | |
| | | DHAM | | not be | | not be | | | | | | |
| | | (52-18) | | calculated) | | calculated) | | | | | | |
| 23 | 134 | Compound | 29.83 | (IC50 | >100.00 | (IC50 | 27.64 | 25.54 | 20.07 | 22.08 | 13.79 | 12.16 |
| | | 1332- | | could | | could | | | | | | |
| | | DHAM | | not be | | not be | | | | | | |
| | | (52-18) | | calculated) | | calculated) | | | | | | |
| 24 | 133 | Compound | 8152.76 | (IC50 | >10000.00 | (IC50 | 5346.9 | 3006.85 | 3812.92 | 4747.19 | 5875.35 | 1526.13 |
| | | 1331- | | could | | could | | | | | | |
| | | DHAM | | not be | | not be | | | | | | |
| | | (50-18) | | calculated) | | calculated) | | | | | | |
| 25 | 132 | Compound | >10000.00 | (IC50 | >10000.00 | <10.00 | >10000.00 | >10000.00 | >10000.00 | >10000.00 | >10000.00 | 5828.02 |
| | | 1330- | | could | | | | | | | | |
| | | DHAM | | not be | | | | | | | | |
| | | (50-18) | | calculated) | | | | | | | | |
| 26 | 180 | Compound | 330.07 | (IC50 | 745.32 | (IC50 | 484.64 | 448.83 | 275.84 | 414 | 746.59 | 137.26 |
| | | 1324- | | could | | could | | | | | | |
| | | DHAM | | not be | | not be | | | | | | |
| | | (52-18) | | calculated) | | calculated) | | | | | | |
| 27 | 179 | Compound | 495.17 | (IC50 | 1155.76 | (IC50 | 678.76 | 544.87 | 388.2 | 625.6 | 1189.14 | 152.16 |
| | | 1323- | 395.32 | could | >1000.00 | could | 578.75 | 469.17 | 306.91 | 524.53 | 974.64 | 167.96 |
| | | DHAM | | not be | | not be | | | | | | |
| | | (52-18) | | calculated) | | calculated | | | | | | |
| | | | | <1.00 | | because | | | | | | |
| | | | | | | value | | | | | | |
| | | | | | | was | | | | | | |
| | | | | | | manually | | | | | | |
| | | | | | | overridden) | | | | | | |
| | | | | | | <1.00 | | | | | | |
| 28 | 178 | Compound | >10000.00 | >10000.00 | >10000.00 | <10.00 | >10000.00 | >10000.00 | >10000.00 | >10000.00 | >10000.00 | >10000.00 |
| | | 1322- | | | | | | | | | | |
| | | DHAM | | | | | | | | | | |
| | | (50-18) | | | | | | | | | | |
| 29 | 177 | Compound | >10000.00 | (IC50 | (IC50 | <10.00 | >10000.00 | >10000.00 | (IC50 | >10000.00 | >10000.00 | >10000.00 |
| | | 1321- | | could | could | | | | could | | | |
| | | DHAM | | not be | not be | | | | not be | | | |
| | | (50-18) | | calculated) | calculated) | | | | calculated) | | | |
| 30 | 116 | Compound | >100.00 | (IC50 | (IC50 | (IC50 | >100.00 | >100.00 | >100.00 | >100.00 | >100.00 | >100.00 |
| | | 1253- | 188.56 | could | could | could | 161.52 | 107.27 | 147 | 127.8 | 107.25 | 114.13 |
| | | DHAM | >100.00 | not be | not be | not be | | | | | | |
| | | batch2 | | calculated) | calculated) | calculated) | | | | | | |
| | | | | (IC50 | 634.28 | (IC50 | >100.00 | >100.00 | >100.00 | >100.00 | >100.00 | >100.00 |
| | | | | could | >100.00 | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 31 | 112 | Compound | 54.84 | <0.10 | 82.48 | (IC50 | 46.15 | 37.23 | 43.96 | 40.95 | 36.4 | 40.7 |
| | | 1249- | | | | could | | | | | | |
| | | DHAM- | | | | not be | | | | | | |
| | | Batch 1 | | | | calculated) | | | | | | |
| 32 | 131 | Compound | 48.5 | (IC50 | 97.64 | <1.00 | 31.51 | 31.3 | 47.82 | 26.32 | 22.56 | 31.5 |
| | | 1265 | | could | | | | | | | | |
| | | | | not be | | | | | | | | |
| | | | | calculated) | | | | | | | | |
| 33 | 122 | Compound | 35.14 | >100.00 | 50.84 | >100.00 | 32.33 | 24.97 | 26.56 | 25.66 | 21.93 | 33.52 |
| | | 1264_ | | | | | | | | | | |
| | | batch1, | | | | | | | | | | |
| | | Compound | | | | | | | | | | |
| | | 1264_ | | | | | | | | | | |
| | | batch2 | | | | | | | | | | |
| 34 | NO | Compound | 11.43 | <0.10 | 18.24 | <0.10 | 10.32 | 8.44 | 8.7 | 8.34 | 6.81 | 9.62 |
| | TIN | 1263, | | | | | | | | | | |
| | TA | Compound | | | | | | | | | | |
| | BLE | 1263- | | | | | | | | | | |
| | | #809 | | | | | | | | | | |
| 35 | 121 | Compound | 14.16 | <0.10 | 19.53 | (IC50 | 12.25 | 9.98 | 10.39 | 10.4 | 10.76 | 10.75 |
| | | 1262_ | | | | could | | | | | | |
| | | batch1, | | | | not be | | | | | | |
| | | Compound | | | | calculated | | | | | | |
| | | 1262_ | | | | because | | | | | | |
| | | batch2 | | | | value | | | | | | |
| | | | | | | was | | | | | | |
| | | | | | | manually | | | | | | |
| | | | | | | overridden) | | | | | | |
| 36 | 130 | Compound | <10.00 | 1402.24 | <10.00 | 512.76 | <10.00 | <10.00 | <10.00 | <10.00 | <10.00 | <10.00 |
| | | 1261 | 2.43 | (IC50 | 3.71 | >100.00 | 2.3 | 1.76 | 1.72 | 1.57 | 1.43 | 1.89 |
| | | | | could | | | | | | | | |
| | | | | not be | | | | | | | | |
| | | | | calculated) | | | | | | | | |
| 37 | 120 | Compound | <10.00 | 475.23 | <10.00 | 187.83 | <10.00 | <10.00 | <10.00 | <10.00 | <10.00 | <10.00 |
| | | 1260_ | 1.77 | (IC50 | 2.21 | 96.79 | 1.74 | 1.47 | 1.43 | 1.31 | 1.27 | 1.57 |
| | | batch1, | | could | | | | | | | | |
| | | compound | | not be | | | | | | | | |
| | | 1260 | | calculated) | | | | | | | | |
| 38 | 118 | Compound | 104.5 | (IC50 | 244.79 | (IC50 | 87.59 | 74.45 | 86.55 | 77.89 | 71.41 | 61.88 |
| | | 1255- | 89.99 | could | >100.00 | could | 78.85 | 71.65 | 82.96 | 73.6 | 75.21 | 62.47 |
| | | DHAM_ | | not be | | not be | | | | | | |
| | | batch1 | | calculated) | | calculated) | | | | | | |
| | | | | (IC50 | | <0.10 | | | | | | |
| | | | | could | | | | | | | | |
| | | | | not be | | | | | | | | |
| | | | | calculated) | | | | | | | | |
| 39 | 115 | Compound | 64.69 | (IC50 | 94.4 | (IC50 | 56.09 | 47.42 | 46.53 | 48.27 | 45.63 | 45.66 |
| | | 1252- | | could | | could | | | | | | |
| | | DHAM | | not be | | not be | | | | | | |
| | | | | calculated) | | calculated) | | | | | | |
| 40 | 114 | Compound | 58.36 | (IC50 | 84.26 | (IC50 | 47.35 | 40.75 | 40.53 | 43.26 | 38.44 | 40.71 |
| | | 1251- | | could | | could | | | | | | |
| | | DHAM_ | | not be | | not be | | | | | | |
| | | batch1 | | calculated) | | calculated) | | | | | | |
| 41 | 113 | Compound | 59.78 | >10000.00 | 80.86 | 5922.86 | 45.55 | 44.09 | 44.61 | 41.49 | 37.18 | 41.17 |
| | | 1250- | 79.24 | (IC50 | >100.00 | (IC50 | 66.41 | 54.71 | 58.08 | 59.46 | 51.81 | 52.95 |
| | | DHAM | | could | | could | | | | | | |
| | | | | not be | | not be | | | | | | |
| | | | | calculated) | | calculated) | | | | | | |
| 42 | 111 | Compound | >10.00 | 550.56 | <10.00 | 222.85 | <10.00 | <10.00 | <10.00 | <10.00 | <10.00 | <10.00 |
| | | 1248- | 1.4 | >100.00 | 2.24 | >100.00 | 1.51 | 1.15 | 1.02 | 1.03 | 0.81 | 1.39 |
| | | DHAM | | | | | | | | | | |
| 43 | 110 | Compound | <10.00 | 567.14 | <10.00 | 221.62 | <10.00 | <10.00 | <10.00 | <10.00 | <10.00 | <10.00 |
| | | 1247- | | | | | | | | | | |
| | | DHAM_ | | | | | | | | | | |
| | | Batch1 | | | | | | | | | | |
| 44 | 119 | Compound | 92.31 | (IC50 | 406.37 | (IC50 | 81.54 | 74.97 | 79.08 | 63.29 | 66.85 | 46.62 |
| | | 1256- | >100.00 | could | >100.00 | could | 82.16 | 80.98 | 98.71 | 82.97 | 89.26 | 66.74 |
| | | DHAM | | not be | | not be | | | | | | |
| | | | | calculated) | | calculated) | | | | | | |
| | | | | <0.10 | | (IC50 | | | | | | |
| | | | | | | could | | | | | | |
| | | | | | | not be | | | | | | |
| | | | | | | calculated) | | | | | | |
| 45 | 117 | Compound | 108.2 | (IC50 | 664.53 | (IC50 | 84.71 | 75.23 | 82.44 | 72.24 | 66.08 | 54.75 |
| | | 1254- | 99.36 | could | >100.00 | could | 88.71 | 77.01 | 92.18 | 90.84 | 79.66 | 61.09 |
| | | DHAM | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 46 | 108 | Compound | 24.43 | (IC50 | 49.99 | (IC50 | 24.5 | 21.59 | 18.81 | 17.67 | 16.41 | 20.58 |
| | | 1197- | | could | | could | | | | | | |
| | | DHAM | | not be | | not be | | | | | | |
| | | (19-56)- | | calculated) | | calculated) | | | | | | |
| | | GQAP- | | | | | | | | | | |
| | | uABT | | | | | | | | | | |
| 47 | 107 | Compound | 49.67 | (IC50 | 65.45 | (IC50 | 38.75 | 35.65 | 33.68 | 32.01 | 31.95 | 36.19 |
| | | 1196- | | could | | could | | | | | | |
| | | DHAM | | not be | | not be | | | | | | |
| | | (19-56)- | | calculated) | | calculated) | | | | | | |
| | | G4- | | | | | | | | | | |
| | | uABT | | | | | | | | | | |
| 48 | 106 | Compound | 29.65 | <0.10 | 54.25 | (IC50 | 24.84 | 17.16 | 17.95 | 21.12 | 12.12 | 22.13 |
| | | 1195- | | | | could | | | | | | |
| | | DHAM | | | | not be | | | | | | |
| | | (19-56)- | | | | calculated) | | | | | | |
| | | G4- | | | | | | | | | | |
| | | uABT | | | | | | | | | | |
| 49 | 109 | Compound | 20.4 | (IC50 | 42.55 | <0.10 | 19.58 | 16.56 | 16.15 | 14.05 | 12.72 | 15.87 |
| | | 1198- | | could | | | | | | | | |
| | | DHAM | | not be | | | | | | | | |
| | | (19-56)- | | calculated) | | | | | | | | |
| | | GQAP- | | | | | | | | | | |
| | | uABT | | | | | | | | | | |
| 50 | 128 | Compound | <1.00 | 171.1 | 1.32 | 71.23 | 1.08 | <1.00 | <1.00 | <1.00 | <1.00 | <1.00 |
| | | 1168- | | | | | | | | | | |
| | | NEW | | | | | | | | | | |
| | | LCB1 | | | | | | | | | | |
| 51 | 128 | Compound | 0.91 | 92.26 | 1.31 | 50.84 | 1.01 | 0.8 | 0.77 | 0.7 | 0.63 | 0.86 |
| | | 1168- | | | | | | | | | | |
| | | NEW | | | | | | | | | | |
| | | LCB1 | | | | | | | | | | |
| 52 | 129 | Compound | 34.8 | (IC50 | 44.81 | (IC50 | 29.5 | 26.01 | 26.82 | 26.64 | 23.26 | 25.04 |
| | | 1135- | | could | | could | | | | | | |
| | | N1- | | not be | | not be | | | | | | |
| | | DHAM | | calculated) | | calculated) | | | | | | |
| | | (18-56) | | | | | | | | | | |
| 54 | 155 | Compound | 12.51 | (IC50 | 61.69 | (IC50 | 11.32 | 9.54 | 10.59 | 10.13 | 10.46 | 7.67 |
| | | 1152- | | could | | could | | | | | | |
| | | N1- | | not be | | not be | | | | | | |
| | | DHAM | | calculated) | | calculated) | | | | | | |
| | | (18-52) | | | | | | | | | | |
| 55 | 125 | Compound | 4.39 | (IC50 | >100.00 | (IC50 | 4.61 | 3.8 | 3.71 | 3.55 | 3.97 | 3.77 |
| | | 1043- | 1.33 | could | 50.36 | could | 1.53 | 1.22 | 1.27 | 1.08 | 1.32 | 1.16 |
| | | N3_batch | 1.05 | not be | 45.54 | not be | 1.2 | 1.03 | 1.1 | <1.00 | 1.14 | 1.08 |
| | | 2 | 1.03 | calculated) | 50.93 | calculated) | 1.16 | <1.00 | 1.04 | <1.00 | 1.03 | 1.08 |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 56 | 126 | Compound | 1.38 | (IC50 | 62.05 | (IC50 | 1.49 | 1.22 | 1.24 | 1.07 | 1.17 | 1.24 |
| | | 1044- | 2.98 | could | >100.00 | could | 3.19 | 2.1 | 2.39 | 2.25 | 2.36 | 2.66 |
| | | N3, | 2.94 | not be | >100.00 | not be | 3.33 | 2.78 | 2.69 | 2.18 | 2.61 | 2.61 |
| | | Compound | <1.00 | calculated) | 52.96 | calculated) | <1.00 | <1.00 | <1.00 | <1.00 | <1.00 | <1.00 |
| | | 1044- | | (IC50 | | (IC50 | | | | | | |
| | | N3_batch | | could | | could | | | | | | |
| | | 2 | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 57 | 124 | Compound | 1.27 | 133.92 | 1.53 | 57 | 1.46 | 1.34 | 1.12 | 1.04 | <1.00 | 1.2 |
| | | 1042- | 1.3 | >100.00 | 1.64 | 62.31 | 1.55 | 1.32 | 1.16 | 1.12 | 0.99 | 1.34 |
| | | N1, | | | | | | | | | | |
| | | Compound | | | | | | | | | | |
| | | 1042- | | | | | | | | | | |
| | | N1_batch | | | | | | | | | | |
| | | 2, | | | | | | | | | | |
| | | Compound | | | | | | | | | | |
| | | 1042_ | | | | | | | | | | |
| | | batch3 | | | | | | | | | | |
| 58 | 123 | Compound | 1.53 | 94.77 | 2.15 | 60.46 | 2.03 | 1.65 | 1.32 | 1.34 | 1.29 | 1.53 |
| | | 1041- | 1.41 | >100.00 | 1.97 | 71.87 | 1.71 | 1.52 | 1.16 | 1.16 | 1.11 | 1.37 |
| | | N1, | | | | | | | | | | |
| | | Compound | | | | | | | | | | |
| | | 1041- | | | | | | | | | | |
| | | N1_Batch | | | | | | | | | | |
| | | 3, | | | | | | | | | | |
| | | Compound | | | | | | | | | | |
| | | 1041- | | | | | | | | | | |
| | | N2_batch | | | | | | | | | | |
| | | 2_2, | | | | | | | | | | |
| | | Compound | | | | | | | | | | |
| | | 1041_ | | | | | | | | | | |
| | | batch3 | | | | | | | | | | |
| 59 | 103 | Compound | 7.84 | (IC50 | 9 | >100.00 | 5.59 | 4.2 | 5.61 | 5.16 | 3.98 | 6.22 |
| | | 1140- | | could | | | | | | | | |
| | | N1- | | not be | | | | | | | | |
| | | DHAM | | calculated) | | | | | | | | |
| | | (18-56), | | | | | | | | | | |
| | | Compound | | | | | | | | | | |
| | | 963- | | | | | | | | | | |
| | | 12 | | | | | | | | | | |
| | | families- | | | | | | | | | | |
| | | 10 | | | | | | | | | | |
| 60 | 102 | Compound | 9.19 | (IC50 | 10.06 | (IC50 | 6.93 | 5.38 | 6.67 | 5.4 | 4.62 | 5.96 |
| | | 962- | | could | | could | | | | | | |
| | | 12 | | not be | | not be | | | | | | |
| | | families- | | calculated) | | calculated) | | | | | | |
| | | 10 | | | | | | | | | | |
| 61 | 181 | Compound | 3.79 | (IC50 | 15.23 | (IC50 | 4.04 | 3.32 | 3.16 | 3.17 | 3.24 | 2.51 |
| | | 920- | | could | | could | | | | | | |
| | | cyclic | | not be | | not be | | | | | | |
| | | peptides | | calculated) | | calculated) | | | | | | |
| 62 | 137 | Compound | 4.68 | (IC50 | 15.63 | (IC50 | 4.57 | 4.46 | 3.88 | 3.5 | 4.04 | 2.91 |
| | | 919- | | could | | could | | | | | | |
| | | cyclic | | not be | | not be | | | | | | |
| | | peptides | | calculated) | | calculated) | | | | | | |
| 63 | 136 | Compound | 4.43 | (IC50 | 17.65 | (IC50 | 4.44 | 4.23 | 3.53 | 3.52 | 4.07 | 2.53 |
| | | 918- | | could | | could | | | | | | |
| | | cyclic | | not be | | not be | | | | | | |
| | | peptides | | calculated) | | calculated) | | | | | | |
| 64 | 176 | Compound | 12.47 | (IC50 | 12.42 | 665.35 | 13.87 | 12.99 | 9.24 | 10.68 | 20.73 | 5.02 |
| | | 874 | | could | | | | | | | | |
| | | | | not be | | | | | | | | |
| | | | | calculated) | | | | | | | | |
| 65 | 175 | Compound | 17.99 | (IC50 | 16.38 | 875.92 | 16.29 | 14.62 | 11.86 | 13.11 | 24.35 | 7.68 |
| | | 872 | | could | | | | | | | | |
| | | | | not be | | | | | | | | |
| | | | | calculated) | | | | | | | | |
| 66 | 138 | Compound | 29.42 | (IC50 | 39.39 | >1000.00 | 32.53 | 33.17 | 25.52 | 26.79 | 55.45 | 12.73 |
| | | 871 | | could | | | | | | | | |
| | | | | not be | | | | | | | | |
| | | | | calculated) | | | | | | | | |
| 67 | 195 | Compound | 0.73 | (IC50 | 68.7 | (IC50 | 1.01 | 0.79 | 0.77 | 0.55 | 0.7 | 0.66 |
| | | 850- | 2.63 | could | >100.00 | could | 2.87 | 1.9 | 2.05 | 1.86 | 1.87 | 2.23 |
| | | LCB3- | 1.15 | not be | 83.8 | not be | 1.32 | 1.09 | 1.02 | 0.9 | 1.03 | 1.16 |
| | | NH2 | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 68 | 184 | Compound | 0.75 | (IC50 | 60.99 | (IC50 | 1 | 0.77 | 0.71 | 0.55 | 0.93 | 0.66 |
| | | 849- | 2.78 | could | >100.00 | could | 3.23 | 2.3 | 2.51 | 2.09 | 2.9 | 2.42 |
| | | LCB3- | 1.18 | not be | 66.97 | not be | 1.37 | 1.13 | 1.06 | 0.95 | 1.31 | 1.1 |
| | | NH2 | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| 69 | 105 | Compound | 0.91 | >100.00 | 1.42 | 64.64 | 1.17 | 1.05 | 0.8 | 0.71 | 0.39 | 0.84 |
| | | 845- | | | | | | | | | | |
| | | LCB1- | | | | | | | | | | |
| | | NH2 | | | | | | | | | | |
| 70 | 104 | Compound | 2.73 | (IC50 | >100.00 | (IC50 | 3.13 | 2.37 | 2.51 | 2.07 | 2.64 | 2.52 |
| | | 840-LCB3 | 1.24 | could | 64.44 | could | 1.3 | 1.06 | 1.06 | 1 | 1.3 | 1.19 |
| | | | 0.99 | not be | 61.15 | not be | 1.16 | 0.9 | 0.83 | 0.87 | 1.18 | 0.92 |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |
| | | | | (IC50 | | (IC50 | | | | | | |
| | | | | could | | could | | | | | | |
| | | | | not be | | not be | | | | | | |
| | | | | calculated) | | calculated) | | | | | | |

### Example 32. Examination of Ligand Binding to Native IgG1

The binding of exemplary compounds to native human IgG1 was measured by Bio-Layer Interferometry (BLI) using the ForteBio Octet® 96e platform (Fremont, CA). The binding assay was performed using the Protein L (ProL) Dip and Read Biosensors (ForteBio 18-5085) in a 96-well black flat bottom assay plate (Greiner Bio-One 655209; Monroe, NC) with the following steps: (1) 60 seconds of baseline; (2) 600 seconds of loading with 50 μg/ml of Native Human IgG1 (Abcam ab184886; Cambridge, UK); (3) 60 seconds of baseline; (4) 180 seconds of association with a dilution series of ligand in Assay Buffer (PBS (Gibco 10010; Grand Island, New York), 0.1% (w/v) BSA (American Bio AB01088-00100; Canton, MA), 0.05% (v/v) Tween 20 (Fisher BioReagents™ BP337-500; Pittsburgh, PA), pH 7.4), consisting of seven concentrations. The eighth tip in the column was run in Assay Buffer to serve as a reference sensor, the signal of which was subtracted from the sample sensors. (5) 180 seconds of dissociation in Assay Buffer.

The data was analyzed with the Octet® Data Analysis HT software. The processed data was globally fit to a 1:1 binding model for determination of the equilibrium dissociation constant (K_{D}) and is shown in Table 7.

### Example 33. Examination of Ligand Binding to SARS-CoV-2 Spike Glycorotein Receptor Binding Domain (RBD)

The binding of exemplary compounds to Spike RBD was measured by BLI using the ForteBio Octet® 96e platform (Fremont, CA). The binding assay was performed using the Streptavidin (SA) Dip and Read Biosensors (ForteBio 18-5019) in a 96-well black flat bottom assay plate (Greiner Bio-One 655209; Monroe, NC) with the following steps: (1) 60 seconds of baseline; (2) 300 seconds of loading with 50 nM Biotinylated SARS-CoV-2 Spike glycoprotein Receptor Binding Domain (Biotinylated SARS-CoV-2 Spike RBD, His, Avitag™ (Acro Biosystems SPD-C82E9; Newark, DE); (3) 60 seconds of baseline; (4) 120 seconds of quenching with 50 μg/ml of Biocytin (Thermo B1592; Waltham, MA); (5) 60 seconds of baseline; (6) 180 seconds of association with a dilution series of ligand in Assay Buffer (PBS (Gibco 10010; Grand Island, New York), 0.1% (w/v) BSA (American Bio AB01088-00100; Canton, MA), 0.05% (v/v) Tween 20 (Fisher BioReagents™ BP337-500; Pittsburgh, PA), pH 7.4), consisting of seven concentrations. The eighth tip in the column was run in Assay Buffer to serve as a reference sensor, the signal of which was subtracted from the sample sensors. (7) 300 seconds of dissociation in Assay Buffer.

The data was analyzed with the Octet® Data Analysis HT software. The processed data was globally fit to a 1:1 binding model for determination of the equilibrium dissociation constant (K_{D}) and is shown in Table 7.

### Example 34. Examination of Ligand Binding to SARS-CoV-2 Omicron Spike Glycoprotein

The binding to Omicron Spike glycoprotein was measured by BLI using the ForteBio Octet® 96e platform (Fremont, CA). The binding assay was performed using the Anti-Penta His (HIS1K) Dip and Read Biosensors (ForteBio 18-5120) in a 96-well black flat bottom assay plate (Greiner Bio-One 655209; Monroe, NC) with the following steps: (1) 60 seconds of baseline; (2) 600 seconds of loading with 50 nM Omicron Spike Glycoprotein (SARS-CoV-2 Spike S1, His Tag (B.1.1.529/Omicron); Acro Biosystems S1N-C₅₂Ha; Newark, DE); (3) 60 seconds of baseline; (4) 180 seconds of association with a dilution series of ligand in Assay Buffer (PBS (Gibco 10010; Grand Island, New York), 0.1% (w/v) BSA (American Bio AB01088-00100; Canton, MA), 0.05% (v/v) Tween 20 (Fisher BioReagents™ BP337-500; Pittsburgh, PA), pH 7.4), consisting of seven concentrations. The eighth tip in the column was run in Assay Buffer to serve as a reference sensor, the signal of which was subtracted from the sample sensors. (5) 180 seconds of dissociation in Assay Buffer.

The data was analyzed using the Octet® Data Analysis HT software. Processed data was globally fit to a 1:1 binding model for determination of the equilibrium dissociation constant (K_{D}) and is shown in Table 26.

### Example 35. Examination of Ligand Binding to SARS-CoV-2 Spike Glycoprotein Receptor Binding Domain (RBD) Proteins by ELISA

Materials. The binding affinity of biotinylated ligands to SARS-CoV-2 Spike Glycoprotein RBD protein was determined by ELISA. The binding assay was performed using the following steps: (1) assay plates (PE B&W Isoplate 96-well Flat Bottom, High Bindi-g - Perkin Elmer 6005580) were coated with 100 ul Anti-6× His tag antibody (Anti-6× His tag Antibody; Abcam ab15149, Mouse Monoclonal; Cambridge, UK) at 5 μg/ml in PBS, incubate 1 hour at 37° C. without shaking. The plates were washed (each wash step of this assay includes four 1-minute washes with 200 ul phosphate-buffered saline-0.05% (w/v) Tween® 20 detergent (PBST)). (2) Plates were blocked with 200 ul PBST-5% BSA (PBSTB; American Bio AB01088-00100; Canton, MA), for an hour at room temperature on a plate shaker (400 rpm). The plates were washed. (3) His-tagged protein (SARS-CoV-2 (COVID-19) S Protein RBD, His Tag; Acro Biosystems SPD-C52H3, 0.6 mg/ml=22.6; Newark, DE) was added, 100 ul at 5 nM in PBSTB, and the plates incubated for 1 hour at room temperature on a plate shaker (200 RPM). The plates were washed. (4) Ligands were added, 100 ul at a concentration range of 0.1 mM to 100 nM in PBSTB, and incubated for 1 hour at room temperature on a plate shaker (200 RPM). The plates were washed. (5) NeutrAvidin™-HRP (Invitrogen 31030; Waltham, MA) is added, 100 ul at a dilution of 1:20,000 in PBSTB, and incubated for 1 hour at room temperature on a plate shaker (200 RPM). The plates were washed. (6) SuperSignal ELISA Pico Chemiluminescent Substrate (Thermo Scientific 37069; Waltham, MA) was added and incubated for 5 minutes at room temperature on a plate shaker (200 RPM). The plates were read on the BioTek® Synergy H1 Microplate Reader (Model H1MFG; Winooski, VT) and the data is shown in Table 26.

TABLE 26
Data for SARS-CoV-2 Spike Glycoprotein Binding and Native IgG1 Binding with Exemplary
Ligands Utilized in Compounds of the Present Disclosure

| | | | | | Octet ® SARS- | ELISA |
|---|---|---|---|---|---|---|
| | | | | Octet ® SARS- | CoV-2 | SARS-CoV-2 |
| | | | | CoV-2 Spike | Omicron | Spike RBD |
| | | | Octet ® Native | RBD Protein | Spike | Protein |
| | Cpd. | | IgG1 Binding | Binding | Glycoprotein | Binding |
| Number | No. | Synonyms | KD (nM) | KD (nM) | Binding KD (nM) | EC₅₀ (nM) |
| 1 | 168 | Compound | <1.0 | 0.463 | | |
| | | 1477-56-18- | <1.0 | | | |
| | | (Peg8)3-uABT | | | | |
| 2 | 167 | Compound | <1.0 | 0.327 | | |
| | | 1476-56-18- | <1.0 | | | |
| | | (PAS)4-uABT | | | | |
| 3 | 166 | Compound | <1.0 | 0.444 | | |
| | | 1475-56-18 | <1.0 | | | |
| | | G8-uABT | | | | |
| 4 | 165 | Compound | 10.2 | <0.0010 | | |
| | | 1474-56-1- | | | | |
| | | (PAS)4-uABT | | | | |
| 5 | 174 | Compound | 20 | 0.787 | | |
| | | 1461 | | | | |
| 6 | 173 | Compound | 18.9 | 0.552 | | |
| | | 1460 | | | | |
| 7 | 172 | Compound | 7.92 | 0.304 | | |
| | | 1459 | | | | |
| 8 | 171 | Compound | 13.7 | 0.126 | | |
| | | 1458 | | | | |
| 9 | 170 | Compound | 16 | 0.272 | | |
| | | 1457 | | | | |
| 10 | 169 | Compound | 17.4 | 0.427 | | |
| | | 1456 | | | | |
| 11 | 147 | Compound | 9.78 | 0.399 | | |
| | | 1455 | | | | |
| 12 | 146 | Compound | 8.13 | 0.11 | | |
| | | 1454 | | | | |
| 13 | 145 | Compound | 26.7 | 0.078 | | |
| | | 1453 | | | | |
| 14 | 144 | Compound | 13.4 | 0.269 | | |
| | | 1452 | | | | |
| 15 | 143 | Compound | 19.3 | 0.362 | | |
| | | 1451 | | | | |
| 16 | 142 | Compound | 22.4 | 0.365 | | |
| | | 1450 | | | | |
| 17 | 141 | Compound | <1.0 pM | 0.662 | | |
| | | 1335- | | 0.662 | | |
| | | DHAM(52- | | | | |
| | | 18)-uABT | | | | |
| 18 | 140 | Compound | 1.73 pM | 109 | | |
| | | 1334- | | 109 | | |
| | | DHAM(50- | | | | |
| | | 18)-uABT | | | | |
| 19 | 139 | Compound | <1.0 | 32 | | |
| | | 1326- | | 32 | | |
| | | DHAM(52- | | | | |
| | | 18)-uABT | | | | |
| 20 | 164 | Compound | <1.0 | Weak Binding | | |
| | | 1325- | | Weak Binding | | |
| | | DHAM(50- | | | | |
| | | 18)-uABT | | | | |
| 21 | 134 | Compound | | 0.15 | | |
| | | 1332- | | | | |
| | | DHAM(52-18) | | | | |
| 22 | 133 | Compound | | 2993 | | |
| | | 1331- | | | | |
| | | DHAM(50-18) | | | | |
| 23 | 132 | Compound | | Weak Binding | | |
| | | 1330- | | | | |
| | | DHAM(50-18) | | | | |
| 24 | 112 | Compound | | 0.325 | | |
| | | 1249-DHAM- | | | | |
| | | Batch 1 | | | | |
| 25 | 131 | Compound | No Binding | Weak Binding | | |
| | | 1265 | | | | |
| 26 | 122 | Compound | No Binding | 3.02 | | |
| | | 1264_batch1, | | 2.19 | | |
| | | Compound | | 2.19 | | |
| | | 1264_batch2 | | | | |
| 27 | NOT IN | Compound | Weak Binding | 6.7 | | |
| | TABLE | 1263, | Weak Binding | 1.08 | | |
| | | Compound | | | | |
| | | 1263-#809 | | | | |
| 28 | 121 | Compound | <1.0 | 3.87 | | |
| | | 1262_batch1, | <1.0 | 0.0416 | | |
| | | Compound | | 0.0416 | | |
| | | 1262_batch2 | | | | |
| 29 | 130 | Compound | 26.5 | 1.8 | | |
| | | 1261 | | | | |
| 30 | 120 | Compound | 11.9 | 0.554 | | |
| | | 1260_batch1, | | | | |
| | | compound | | | | |
| | | 1260 | | | | |
| 31 | 118 | Compound | <1.0 | 1.68 | | |
| | | 1255- | | | | |
| | | DHAM_batch | | | | |
| | | 1 | | | | |
| 32 | 115 | Compound | <1.0 | <1.0 nM | | |
| | | 1252-DHAM | | | | |
| 33 | 114 | Compound | <1.0 | 0.231 | | |
| | | 1251- | | | | |
| | | DHAM_batch | | | | |
| | | 1 | | | | |
| 34 | 113 | Compound | 0.00139 | <1.0 nM | | |
| | | 1250-DHAM | | | | |
| 35 | 111 | Compound | 13.9 | 0.3 | | |
| | | 1248-DHAM | | | | |
| 36 | 110 | Compound | 18.1 | 0.195 | | |
| | | 1247- | | | | |
| | | DHAM_Batch | | | | |
| | | 1 | | | | |
| 37 | 119 | Compound | <1.0 | 5.11 nM | | |
| | | 1256-DHAM | | | | |
| 38 | 117 | Compound | <1.0 | 4.66 nM | | |
| | | 1254-DHAM | | No Binding | | |
| 39 | 108 | Compound | <1.0 | <1.0 pM | | |
| | | 1197- | | | | |
| | | DHAM(19-56)- | | | | |
| | | GQAP-uABT | | | | |
| 40 | 107 | Compound | <1.0 | <1.0 pM | | |
| | | 1196- | | | | |
| | | DHAM(19-56)- | | | | |
| | | G4-uABT | | | | |
| 41 | 106 | Compound | <1.0 | <1.0 pM | | |
| | | 1195- | | | | |
| | | DHAM(19-56)- | | | | |
| | | G4-uABT | | | | |
| 43 | 109 | Compound | <1.0 | <1.0 pM | | |
| | | 1198- | | | | |
| | | DHAM(19-56)- | | | | |
| | | GQAP-uABT | | | | |
| 44 | 128 | Compound | 8.3 | 0.653 nM | | |
| | | 1168-NEW | | | | |
| | | LCB1 | | | | |
| 45 | 159 | Compound | No Binding | 4.1 nM | | |
| | | 1172-N1- | | | | |
| | | DHAM(18-56) | | | | |
| 46 | 161 | Compound | 0.00743 | 0.398 nM | | |
| | | 1171-N1- | 0.00546 | 0.395 nM | | |
| | | DHAM(18- | | | | |
| | | 56)-btch2 | | | | |
| 47 | 160 | Compound | 0.00941 | <1.0 nM | | |
| | | 1170-N1- | 0.0121 | 1.2 pM | | |
| | | DHAM(18- | | | | |
| | | 56)-batch2 | | | | |
| 48 | 129 | Compound | 0.00107 | <1.0 nM | | |
| | | 1135-N1- | | | | |
| | | DHAM(18-56) | | | | |
| 50 | 255 | Compound | | Non-specific | | |
| | | 1194-LCB1- | | Binding | | |
| | | 2020, | | Non-specific | | |
| | | Compound | | Binding | | |
| | | 1194-LCB1- | | | | |
| | | 2021 | | | | |
| 51 | 208 | Compound | | | | No Binding |
| | | 1193-LCB1 | | | | |
| 52 | 254 | Compound | | Non-specific | | |
| | | 1192-LCB1 | | Binding | | |
| 53 | 207 | Compound | | Non-specific | | |
| | | 1191-LCB1 | | Binding | | |
| 54 | 163 | Compound | 0.0626 | 0.505 nM | | |
| | | 1174-N1- | | | | |
| | | DHAM(18-56) | | | | |
| 55 | 162 | Compound | <1.0 | 1.07 nM | | |
| | | 1173-N1- | 0.00137 | 1.26 nM | | |
| | | DHAM(18- | | | | |
| | | 56)_Batch1, | | | | |
| | | Compound | | | | |
| | | 1173-N1- | | | | |
| | | DHAM(18- | | | | |
| | | 56)_Batch2 | | | | |
| 56 | 156 | Compound | 2.28 | 505 nM | | |
| | | 1153-N1- | | | | |
| | | DHAM(18-48) | | | | |
| 57 | 155 | Compound | <1.0 | 2.1 nM | | |
| | | 1152-N1- | | 2.69 | | |
| | | DHAM(18-52) | | | | |
| 58 | 154 | Compound | <1.0 | 120 nM | | |
| | | 1151-N1- | <1.0 | 198 nM | | |
| | | DHAM(23- | <1.0 | | | |
| | | 56)_Batch1, | | | | |
| | | Compound | | | | |
| | | 1151-N1- | | | | |
| | | DHAM(23- | | | | |
| | | 56)_Batch2 | | | | |
| 59 | 153 | Compound | <1.0 | Weak Binding | | |
| | | 1150-N1- | <1.0 | | | |
| | | DHAM(28-56) | | | | |
| 60 | 152 | Compound | <1.0 | Weak Binding | | |
| | | 1149-N1- | 0.00108 | | | |
| | | DHAM(32-56) | | | | |
| 61 | 151 | Compound | <1.0 | Weak Binding | | |
| | | 1148-N1- | <1.0 | | | |
| | | DHAM(37-56) | | | | |
| 62 | 150 | Compound | <1.0 | 0.117 nM | | |
| | | 1147-N1- | <1.0 | | | |
| | | DHAM(18-57) | | | | |
| 63 | 148 | Compound | <1.0 | <1.0 nM | | |
| | | 1134-N1- | 0.00105 | | | |
| | | DHAM(18-56) | | | | |
| 64 | 149 | Compound | 0.00148 | <1.0 nM | | |
| | | 1133-N1- | <1.0 | <1.0 | | |
| | | DHAM(18-56) | <1.0 | | | |
| 65 | 187 | Compound | | | | 0.37 |
| | | 981-Strata 1.5 | | | | |
| 66 | 158 | Compound | | 1.52 | | |
| | | 1169-NEW | | | | |
| | | LCB1 | | | | |
| 67 | 233 | Compound | | 9148 | | |
| | | 1160-N1- | | | | |
| | | DHAM(18-48) | | | | |
| 68 | 232 | Compound | | 1.91 | | |
| | | 1159-N1- | | | | |
| | | DHAM(18-53) | | | | |
| 69 | 231 | Compound | | 147 | | |
| | | 1158-N1- | | | | |
| | | DHAM(22-56) | | | | |
| 70 | 230 | Compound | | No Binding | | |
| | | 1157-N1- | | | | |
| | | DHAM(28-56) | | | | |
| 71 | 229 | Compound | | No Binding | | |
| | | 1156-N1- | | | | |
| | | DHAM(32-56) | | | | |
| 72 | 228 | Compound | | No Binding | | |
| | | 1155-N1- | | | | |
| | | DHAM(37-56) | | | | |
| 73 | 227 | Compound | | 0.0601 | | |
| | | 1154-N1- | | | | |
| | | DHAM(18-57) | | | | |
| 74 | 194 | Compound | | 0.344 | | |
| | | 1141-N1- | | | | |
| | | DHAM(18-56) | | | | |
| 75 | 226 | Compound | | 0.0567 | | |
| | | 1138-N1- | | | | |
| | | DHAM(18-56) | | | | |
| 76 | 193 | Compound | | | | 4.6 |
| | | 1085- | | | | |
| | | LCB1TurnMut | | | | |
| 77 | 202 | Compound | | 0.00162 | | |
| | | 1084- | | | | |
| | | LCB1TurnMut | | | | |
| 78 | 192 | Compound | | <1.0 pM | | |
| | | 1083- | | | | |
| | | LCB1TurnMut | | | | |
| 79 | 222 | Compound | | | | 0.79 |
| | | 1081- | | | | |
| | | LCB1TurnMut | | | | |
| 80 | 240 | Compound | | <1.0 pM | | |
| | | 1080- | | | | |
| | | LCB1TurnMut | | | | |
| 81 | 221 | Compound | | 0.0116 | | |
| | | 1079- | | | | |
| | | LCB1TurnMut | | | | |
| 82 | NA | Compound | | 1.05 | | |
| | | 1076- | | | | |
| | | LCB1TurnMut | | | | |
| 83 | 191 | Compound | | 28.1 | | |
| | | 1075- | | | | |
| | | LCB1TurnMut | | | | |
| 84 | 125 | Compound | | 42.6 | | |
| | | 1043- | | | | |
| | | N3_batch2 | | | | |
| 85 | 220 | Compound | | | | 2.55 |
| | | 1069-(Biot- | | | | |
| | | 2435-6) | | | | |
| 86 | 239 | Compound | | 6.91 | | |
| | | 1068-(Ac- | | | | |
| | | 2435-6) | | | | |
| 87 | 219 | Compound | | 3.42 | | |
| | | 1067-(NH2- | | | | |
| | | 2435-6) | | | | |
| 88 | 218 | Compound | | | | 2.38 |
| | | 1065-(Biot- | | | | |
| | | 2435-5) | | | | |
| 89 | 238 | Compound | | 4.72 | | |
| | | 1064-(Ac- | | | | |
| | | 2435-5) | | | | |
| 90 | 217 | Compound | | 3.81 | | |
| | | 1063-(NH2- | | | | |
| | | 2435-5) | | | | |
| 91 | 216 | Compound | | | | 1.11 |
| | | 1057-(Biot- | | | | |
| | | 2435-3) | | | | |
| 92 | 237 | Compound | | 0.253 | | |
| | | 1056-(Ac- | | | | |
| | | 2435-3) | | | | |
| 93 | 256 | Compound | | 0.113 | | |
| | | 1055-(NH2- | | | | |
| | | 2435-3) | | | | |
| 94 | 214 | Compound | | | | 0.78 |
| | | 1053-(Biotin- | | | | |
| | | 2435-2) | | | | |
| 95 | 236 | Compound | | 0.239 | | |
| | | 1052-(Ac- | | | | |
| | | 2435-2) | | | | |
| 96 | 215 | Compound | | 0.837 | | |
| | | 1051-(NH2- | | | | |
| | | 2435-2) | | | | |
| 97 | 206 | Compound | | No Binding | | |
| | | 1045- | | Weak Binding | | |
| | | 859acid_batch | | No Binding | | |
| | | 1, Compound | | | | |
| | | 1045- | | | | |
| | | 859acid_batch | | | | |
| | | 2, Compound | | | | |
| | | 1045- | | | | |
| | | 859acid_batch | | | | |
| | | 3 | | | | |
| 98 | 244 | Compound | | 56.9 | | |
| | | 1007-NEW | | | | |
| | | LCB3-dia- | | | | |
| | | PEG-acid | | | | |
| 99 | 251 | Compound | | | | 0.92 |
| | | 993-Strata 1.5- | | | | |
| | | Biotin | | | | |
| 101 | 126 | Compound | | 3.1 | | |
| | | 1044-N3, | | | | |
| | | Compound | | | | |
| | | 1044- | | | | |
| | | N3_batch2 | | | | |
| 102 | 124 | Compound | 7.17 | 0.38 | Weak Binding | |
| | | 1042-N1, | 6.28 | | | |
| | | Compound | | | | |
| | | 1042- | | | | |
| | | N1_batch2, | | | | |
| | | Compound | | | | |
| | | 1042_batch3 | | | | |
| 103 | 123 | Compound | 13.7, 6.02 | 1.08 | | |
| | | 1041-N1, | 6.28 | | | |
| | | Compound | | | | |
| | | 1041- | | | | |
| | | N1_Batch 3, | | | | |
| | | Compound | | | | |
| | | 1041- | | | | |
| | | N2_batch2_2, | | | | |
| | | Compound | | | | |
| | | 1041_batch3 | | | | |
| 104 | 200 | Compound | | 0.27 | | |
| | | 984-Strata 1.5 | | | | |
| 105 | 190 | Compound | | 1.71 | | |
| | | 983-Strata 1.5 | | | | |
| 106 | 199 | Compound | | 0.25 | | |
| | | 980-Strata 1.5 | | | | |
| 107 | 189 | Compound | | 2.58 | | |
| | | 979-Strata 1.5 | | | | |
| 108 | 198 | Compound | | 1.13 | | |
| | | 976-Strata | | 99 | | |
| | | 1.5_Batch1, | | | | |
| | | Compound | | | | |
| | | 976-Strata | | | | |
| | | 1.5_Batch2 | | | | |
| 109 | 188 | Compound | | 16.2 | | |
| | | 975-Strata 1.5 | | | | |
| 110 | 224 | Compound | | 0.011 | | |
| | | 947-12 | | | | |
| | | families-6 | | | | |
| 111 | 223 | Compound | | 0.198 | | |
| | | 935-12 | | | | |
| | | families-3 | | | | |
| 112 | 203 | Compound | | 1.92 | | |
| | | 934-12 | | | | |
| | | families-3 | | | | |
| 113 | 204 | Compound | | 2.52 | | |
| | | 930-12 | | | | |
| | | families-2 | | | | |
| 114 | 196 | Compound | | 0.45 | | |
| | | 887-Strata 1 | | | | |
| 115 | 235 | Compound | | No Binding | | |
| | | 971-12 | | | | |
| | | families-12 | | | | |
| 116 | 212 | Compound | | No Binding | | |
| | | 970-12 | | | | |
| | | families-12 | | | | |
| 117 | 234 | Compound | | No Binding | | |
| | | 967-12 | | | | |
| | | families-11 | | | | |
| 118 | 210 | Compound | | No Binding | | |
| | | 966-12 | | | | |
| | | families-11 | | | | |
| 119 | 103 | Compound | No Binding | 0.2 | | |
| | | 1140-N1- | 0.0076 | 0.330 nM | | |
| | | DHAM(18- | 0.00274 | 0.107 nM | | |
| | | 56), | 14 | | | |
| | | Compound | No Binding | | | |
| | | 963-12 | | | | |
| | | families-10 | | | | |
| 120 | 102 | Compound | | 0.76 | | |
| | | 962-12 | | | | |
| | | families-10 | | | | |
| 121 | 181 | Compound | | <0.001 | | |
| | | 920-cyclic | | | | |
| | | peptides | | | | |
| 122 | 186 | Compound | | 1.79 | | |
| | | 886-Strata 1 | | | | |
| 123 | 137 | Compound | | <1.0 | | |
| | | 919-cyclic | | | | |
| | | peptides | | | | |
| 124 | 136 | Compound | | <1.0 | | |
| | | 918-cyclic | | <1.0 | | |
| | | peptides | | | | |
| 125 | 176 | Compound | | 6.3 | | |
| | | 874 | | | | |
| 126 | 175 | Compound | | 12.2 | | |
| | | 872 | | | | |
| 127 | 138 | Compound | | 17.3 | | |
| | | 871 | | | | |
| 128 | 185 | Compound | | <1.0 | | |
| | | 851-LCB3- | | | | |
| | | NH2 | | | | |
| 129 | 195 | Compound | | 3.36 | | |
| | | 850-LCB3- | | | | |
| | | NH2 | | | | |
| 130 | 184 | Compound | | 3.05 | | |
| | | 849-LCB3- | | | | |
| | | NH2 | | | | |
| 131 | 183 | Compound | | <1.0 | | |
| | | 846-LCB1- | | | | |
| | | NH2 | | | | |
| 132 | 105 | Compound | | 2.29 | | |
| | | 845-LCB1- | | | | |
| | | NH2 | | | | |
| 133 | 182 | Compound | | 2.48 | | |
| | | 844-LCB1- | | 1.98 | | |
| | | NH2_Batch1, | | | | |
| | | Compound | | | | |
| | | 844-LCB1- | | | | |
| | | NH2_Batch2 | | | | |
| 134 | 104 | Compound | | 3.26 | | |
| | | 840-LCB3 | | | | |
| 135 | 225 | Compound | | 1.35 | | |
| | | 819- | | | | |
| | | LCB1NEW1, | | | | |
| | | Compound | | | | |
| | | 819_batch2 | | | | |
| 136 | 205 | Compound | | 3.47 | | |
| | | 818- | | | | |
| | | LCB1NEW1 | | | | |

### Example 36. Examination of Antibody-Dependent Cellular Phagocytosis (ADCP)

The ability of the compounds to mediate antibody-dependent cellular phagocytosis (ADCP) was assessed using RAW 264.7 macrophages and Spike RBD coated magnetic beads (https://www.acrobiosystems.com/P3194-SARS-CoV-2-Spike-RBD-coupled-Magnetic-Beads.html). Spike RBD coated magnetic beads (3 ul per well) were added to each well of a round bottom 96-well plates. RAW264.7 cells (50,000 cells per well) were added to the beads. Next, the compounds were administered to predetermined final concentrations. The cell-bead mixtures were incubated at 37° C. for 2 hours. The mixtures were then analyzed by flow cytometry. The frequency of free beads was used as a readout with the data shown in Table 27.

TABLE 27
SARS-COV-2 ADCP Assay Data

| | | | ADCP |
|---|---|---|---|
| Number | Cpd. No. | Synonyms | EC₅₀ (nM) |
| 1 | 168 | Compound 1477-56-18 - (Peg8)3-uABT | 0.067 |
| 2 | 167 | Compound 1476-56-18 - (PAS)4-uABT | 0.11 |
| 3 | 166 | Compound 1475-56-18 - G8-uABT | 0.077 |
| 4 | 165 | Compound 1474-56-1 - (PAS)4-uABT | 0.07 |
| 5 | 174 | Compound 1461 | 0.044 |
| 6 | 173 | Compound 1460 | 0.35 |
| 7 | 172 | Compound 1459 | 0.87 |
| 8 | 171 | Compound 1458 | 0.41 |
| 9 | 170 | Compound 1457 | 0.95 |
| 10 | 169 | Compound 1456 | 0.53 |
| 11 | 147 | Compound 1455 | 0.43 |
| 12 | 146 | Compound 1454 | 0.27 |
| 13 | 145 | Compound 1453 | 0.52 |
| 14 | 144 | Compound 1452 | 0.61 |
| 15 | 143 | Compound 1451 | 0.75 |
| 16 | 142 | Compound 1450 | 0.6 |
| 17 | 141 | Compound 1335-DHAM(52-18)-uABT | >100 |
| 18 | 140 | Compound 1334-DHAM(50-18)-uABT | >100 |
| 19 | 139 | Compound 1326-DHAM(52-18)-uABT | 30 |
| 20 | 164 | Compound 1325-DHAM(50-18)-uABT | >100 |
| 21 | 116 | Compound 1253-DHAM_batch2 | 0.061 |
| | | | 0.16 |
| 22 | 112 | Compound 1249-DHAM-Batch 1 | 2 |
| | | | 1.7 |
| 23 | 131 | Compound 1265 | >100 |
| | | | 8.1 |
| 24 | 122 | Compound 1264_batch1, Compound | >100 |
| | | 1264_batch2 | >100 |
| 25 | NOT IN | Compound 1263, Compound 1263-#809 | 2.4 |
| | TABLE | | 1.8 |
| | | | 2.2 |
| | | | 1.2 |
| 26 | 121 | Compound 1262_batch1, Compound | 3.7 |
| | | 1262_batch2 | 3.3 |
| 27 | 130 | Compound 1261 | 1.3 |
| | | | 1.5 |
| 28 | 120 | Compound 1260_batch1, compound 1260 | 1.9 |
| | | | 1.8 |
| 29 | 118 | Compound 1255-DHAM_batch1 | 1.1 |
| | | | 1.1 |
| 30 | 115 | Compound 1252-DHAM | 1.4 |
| | | | 1.3 |
| 31 | 114 | Compound 1251-DHAM_batch1 | 1.6 |
| | | | 1.1 |
| 32 | 113 | Compound 1250-DHAM | 2.2 |
| | | | 1.6 |
| 33 | 111 | Compound 1248-DHAM | 0.18 |
| | | | 0.12 |
| | | | 1.3 |
| | | | 1.2 |
| 34 | 110 | Compound 1247-DHAM_Batch1 | 1.5 |
| | | | 1.2 |
| 35 | 119 | Compound 1256-DHAM | 10 |
| | | | 0.0078 |
| | | | >100 |
| | | | >100 |
| 36 | 117 | Compound 1254-DHAM | 0.031 |
| | | | 0.38 |
| | | | >100 |
| | | | >100 |
| 37 | 108 | Compound 1197-DHAM(19-56) -GQAP- | 0.32 |
| | | uABT | 0.33 |
| | | | >100 |
| | | | >100 |
| 38 | 107 | Compound 1196-DHAM(19-56) -G4-uABT | 2.9 |
| | | | 0.81 |
| | | | >100 |
| | | | >100 |
| 39 | 106 | Compound 1195-DHAM(19-56) -G4-uABT | 0.032 |
| | | | 0.46 |
| | | | >100 |
| | | | >100 |
| 40 | NOT | Compound 1144-N1-DHAM(18-56) | >100 |
| | NUMBERED | | >100 |
| | IN TABLE | | |
| 41 | 109 | Compound 1198-DHAM(19-56) -GQAP- | 100 |
| | | uABT | 0.19 |
| | | | >100 |
| | | | >100 |
| 42 | 128 | Compound 1168-NEW LCB1 | 0.045 |
| | | | 0.052 |
| 43 | 159 | Compound 1172-N1-DHAM(18-56) | >100 |
| | | | 0.03 |
| | | | 0.017 |
| | | | >100 |
| 44 | 161 | Compound 1171-N1-DHAM(18-56)-btch2 | 0.0092 |
| | | | 0.0097 |
| | | | 0.0077 |
| | | | >100 |
| 45 | 160 | Compound 1170-N1-DHAM(18-56)-batch2 | 0.0017 |
| | | | 0.02 |
| | | | 0.0023 |
| | | | >100 |
| 46 | 129 | Compound 1135-N1-DHAM(18-56) | 0.0016 |
| | | | 0.022 |
| | | | 0.012 |
| | | | >100 |
| 48 | 208 | Compound 1193-LCB1 | 1.1 |
| 49 | 163 | Compound 1174-N1-DHAM(18-56) | 0.17 |
| 50 | 162 | Compound 1173-N1-DHAM(18- | 2 |
| | | 56)_Batch1, Compound 1173-N1- | >100 |
| | | DHAM(18-56)_Batch2 | |
| 51 | 156 | Compound 1153-N1-DHAM(18-48) | 0.26 |
| 52 | 155 | Compound 1152-N1-DHAM(18-52) | 7.4 |
| | | | 1.1 |
| | | | 2.1 |
| 53 | 154 | Compound 1151-N1-DHAM(23- | 1.6 |
| | | 56)_Batch1, Compound 1151-N1- | |
| | | DHAM(23-56)_Batch2 | |
| 54 | 153 | Compound 1150-N1-DHAM(28-56) | 7.4 |
| 55 | 152 | Compound 1149-N1-DHAM(32-56) | 3 |
| 56 | 151 | Compound 1148-N1-DHAM(37-56) | 2 |
| 57 | 150 | Compound 1147-N1-DHAM(18-57) | 1.6 |
| 58 | 148 | Compound 1134-N1-DHAM(18-56) | 2.4 |
| 59 | 149 | Compound 1133-N1-DHAM(18-56) | 2.4 |
| 60 | 187 | Compound 981-Strata 1.5 | 2.7 |
| 61 | 187 | Compound 981-Strata 1.5 | 1.3 |
| 62 | 158 | Compound 1169-NEW LCB1 | 7.2 |
| 63 | 158 | Compound 1169-NEW LCB1 | 1.5 |
| 64 | 193 | Compound 1085-LCB1TurnMut | 0.46 |
| 65 | 193 | Compound 1085-LCB1TurnMut | 5.4 |
| 66 | 222 | Compound 1081-LCB1TurnMut | 1 |
| 67 | 222 | Compound 1081-LCB1TurnMut | 2.6 |
| 68 | 125 | Compound 1043-N3_batch2 | 0.039 |
| 69 | 125 | Compound 1043-N3_batch2 | 0.084 |
| 70 | 125 | Compound 1043-N3_batch2 | 0.046 |
| 71 | 125 | Compound 1043-N3_batch2 | 0.27 |
| 72 | 125 | Compound 1043-N3_batch2 | 1.8 |
| 73 | 220 | Compound 1069-(Biot-2435-6) | 0.068 |
| 74 | 220 | Compound 1069-(Biot-2435-6) | 0.12 |
| 75 | 218 | Compound 1065-(Biot-2435-5) | 0.13 |
| 76 | 218 | Compound 1065-(Biot-2435-5) | 0.18 |
| 77 | 216 | Compound 1057-(Biot-2435-3) | 0.035 |
| 78 | 216 | Compound 1057-(Biot-2435-3) | 0.26 |
| 79 | 214 | Compound 1053-(Biotin-2435-2) | 0.066 |
| 80 | 214 | Compound 1053-(Biotin-2435-2) | 0.38 |
| 81 | 251 | Compound 993-Strata 1.5-Biotin | 0.039 |
| 82 | 251 | Compound 993-Strata 1.5-Biotin | 0.35 |
| 83 | 126 | Compound 1044-N3, Compound 1044- | 0.081 |
| | | N3_batch2 | |
| 84 | 126 | Compound 1044-N3, Compound 1044- | 0.18 |
| | | N3_batch2 | |
| 85 | 126 | Compound 1044-N3, Compound 1044- | 0.037 |
| | | N3_batch2 | |
| 86 | 126 | Compound 1044-N3, Compound 1044- | 1.5 |
| | | N3_batch2 | |
| 87 | 126 | Compound 1044-N3, Compound 1044- | 6.6 |
| | | N3_batch2 | |
| 88 | 126 | Compound 1044-N3, Compound 1044- | 0.56 |
| | | N3_batch2 | |
| 89 | 126 | Compound 1044-N3, Compound 1044- | 1.7 |
| | | N3_batch2 | |
| 90 | 124 | Compound 1042-N1, Compound 1042- | 1.6 |
| | | N1_batch2, Compound 1042_batch3 | |
| 91 | 124 | Compound 1042-N1, Compound 1042- | 0.12 |
| | | N1_batch2, Compound 1042_batch3 | |
| 92 | 124 | Compound 1042-N1, Compound 1042- | 0.019 |
| | | N1_batch2, Compound 1042_batch3 | |
| 93 | 124 | Compound 1042-N1, Compound 1042- | 0.43 |
| | | N1_batch2, Compound 1042_batch3 | |
| 94 | 124 | Compound 1042-N1, Compound 1042- | 0.4 |
| | | Nl_batch2, Compound 1042_batch3 | |
| 95 | 124 | Compound 1042-N1, Compound 1042- | 7 |
| | | N1_batch2, Compound 1042_batch3 | |
| 96 | 124 | Compound 1042-N1, Compound 1042- | 0.034 |
| | | N1_batch2, Compound 1042_batch3 | |
| 97 | 124 | Compound 1042-N1, Compound 1042- | 0.017 |
| | | N1_batch2, Compound 1042_batch3 | |
| 98 | 124 | Compound 1042-N1, Compound 1042- | 0.016 |
| | | N1_batch2, Compound 1042_batch3 | |
| 99 | 124 | Compound 1042-N1, Compound 1042- | 0.048 |
| | | N1_batch2, Compound 1042_batch3 | |
| 100 | 124 | Compound 1042-N1, Compound 1042- | 0.014 |
| | | N1_batch2, Compound 1042_batch3 | |
| 101 | 124 | Compound 1042-N1, Compound 1042- | 3.7 |
| | | N1_batch2, Compound 1042_batch3 | |
| 102 | 124 | Compound 1042-N1, Compound 1042- | 3.9 |
| | | N1_batch2, Compound 1042_batch3 | |
| 103 | 124 | Compound 1042-N1, Compound 1042- | 6.4 |
| | | N1_batch2, Compound 1042_batch3 | |
| 104 | 124 | Compound 1042-N1, Compound 1042- | 0.25 |
| | | N1_batch2, Compound 1042_batch3 | |
| 105 | 124 | Compound 1042-N1, Compound 1042- | 0.025 |
| | | N1_batch2, Compound 1042_batch3 | |
| 106 | 124 | Compound 1042-N1, Compound 1042- | 0.047 |
| | | N1_batch2, Compound 1042_batch3 | |
| 107 | 124 | Compound 1042-N1, Compound 1042- | 0.088 |
| | | N1_batch2, Compound 1042_batch3 | |
| 108 | 124 | Compound 1042-N1, Compound 1042- | 1.8 |
| | | N1_batch2, Compound 1042_batch3 | |
| 109 | 124 | Compound 1042-N1, Compound 1042- | 0.43 |
| | | N1_batch2, Compound 1042_batch3 | |
| 110 | 123 | Compound 1041-N1, Compound 1041- | 0.17 |
| | | N1_Batch 3, Compound 1041-N2_batch2_2, | |
| | | Compound 1041_batch3 | |
| 111 | 123 | Compound 1041-N1, Compound 1041- | 6.9 |
| | | N1_Batch 3, Compound 1041-N2_batch2_2, | |
| | | Compound 1041_batch3 | |
| 112 | 123 | Compound 1041-N1, Compound 1041- | 0.61 |
| | | N1_Batch 3, Compound 1041-N2_batch2 2, | |
| | | Compound 1041_batch3 | |
| 113 | 123 | Compound 1041-N1, Compound 1041- | 2.3 |
| | | N1_Batch 3, Compound 1041-N2_batch2_2, | |
| | | Compound 1041_batch3 | |
| 114 | 214 | Compound 972-12 families-12 | >1.0E+03 |
| 115 | 211 | Compound 968-12 families-11 | 2000 |
| 116 | 209 | Compound 964-12 families-10 | 2.1 |
| 117 | 209 | Compound 964-12 families-10 | 4 |
| 118 | 103 | Compound 1140-N1-DHAM(18-56), | >100 |
| | | Compound 963-12 families-10 | |
| 119 | 103 | Compound 1140-N1-DHAM(18-56), | 5.6 |
| | | Compound 963-12 families-10 | |
| 120 | 181 | Compound 920-cyclic peptides | 2.1 |
| 121 | 181 | Compound 920-cyclic peptides | 3.1 |
| 122 | 185 | Compound 851-LCB3-NH2 | 1.4 |
| 123 | 185 | Compound 851-LCB3-NH2 | 19 |
| 124 | 183 | Compound 846-LCB1-NH2 | 0.59 |
| 125 | 183 | Compound 846-LCB1-NH2 | 0.63 |

Novel bifunctional molecules/compounds that modulate immune activities, e.g., for treatment of coronavirus infections, such as severe acute respiratory syndrome coronavirus 2 (SARS-CoV-2) infections or Coronavirus Disease 19 (COVID-19), pharmaceutical compositions comprising the same, and method of making and using the same, are described herein. The bifunctional molecules/compounds of the present disclosure are useful to enhance activities of antibodies, e.g., monoclonal antibodies.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A compound or antibody recruiting molecule (ARM), or a pharmaceutically acceptable salt thereof, having the chemical structure: 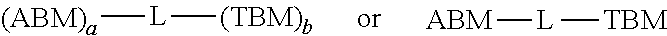
where
(ABM) is an antibody binding moiety or an antibody binding terminus (ABT)) that binds to one or more antibodies or fragments thereof,
a is 1, 2, 3, 4, 5, 6, 7, 8, 9, or 10 (e.g., 1, 2, or 3, preferably 1);
(TBM) is a target binding moiety or target binding terminus (TBT)) that binds the spike glycoprotein of a coronavirus (e.g., severe acute respiratory syndrome coronavirus 2 (SARS-CoV-2)),
b is 1, 2, 3, 4, 5, 6, 7, 8, 9, or 10 (e.g., 1, 2, or 3, preferably 1); and
L is an optional a linker moiety (L) that covalently links the one or more ABMs and the one or more TBMs.

2. The compound of claim 1, or a pharmaceutically acceptable salt thereof, having the chemical structure:
C. 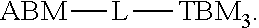
the compound of claim 1, wherein:
a is 1, 2, or 3; and b is 1, 2, or 3.

4. The compound of any one of claims 1-3, wherein one or more (e.g., each) ABM is a peptide, one or more (e.g., each) TBM is a peptide, or a combination thereof.

5. The compound of claim 4, wherein
the N-terminus of the ABM, the TBM, or both are acetylated (—COCH₃);
the C-terminus of the ABM, the TBM, or both are amidated (—NH₂); or a combination thereof
(C) The compound of any one of claims 1-5, or a pharmaceutically acceptable salt thereof, wherein each TBM or target binding moiety independently is or comprises (A), (B), or (CA) an amino acid sequence X^{T1}-X^{T59}:
X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}X^{T42}X^{T43}X^{T44}X^{T45}X^{T46}X^{T47}X^{T48}X^{T49}X^{T50}X⁵¹X^{T52}X^{T53}X^{T54}X^{T55}X^{T56}X^{T57}X^{T58}X^{T59}, wherein:
each of X^{T1} through X^{T59} is an independently selected amino acid residue; and
optionally one or more of X^{T1}X^{T10}, or one or more of X^{T28}-X^{T59} are absent, optionally the X^{T1}-X^{T59} sequence comprises 1, 2, 3 or 4 additional amino acids bound at any point between X^{T1} and X^{T59} where no more than 2 of the additional amino acid are consecutive;
when X^{T1}-X^{T5} are absent, then X^{T11}-X^{T40} are present;
when X^{T28}-X^{T59} are absent, then X^{T1}-X^{T10} are present;
X^{T1} is absent or a hydrophobic amino acid (e.g., an amino acid residue that has a hydrophobic side chain), such as Ala/A, Aib, or an isosterically equivalent amino acid;
X^{T2} is absent or a polar uncharged amino acid (e.g., an amino acid residue with a polar uncharged side chain, such as at physiological pH about 7.4), such as Ser/S or an isosterically equivalent amino acid;
X^{T3} is
absent,
a negatively charged amino acid (e.g., an amino acid residue with a negatively charged side chain, such as at physiological pH about 7.4), such as Asp/D, D-Asp/dD, or an isosterically equivalent amino acid, or
a hydrophobic amino acid, such as Ala/A, D-Ala/dA, or an isosterically equivalent amino acid;
X^{T4} is
Absent,
a positively charged amino acid (e.g., an amino acid residue with a positively charged side chain, such as at physiological pH of about 7.4), such as Lys/K or an isosterically equivalent amino acid; or
a hydrophobic amino acid, such as Abu, or an isosterically equivalent amino acid;
X^{T5} is absent or a negatively charges amino acid, such as Glu/E or an isosterically equivalent amino acid;
X^{T6} is absent or a hydrophobic amino acid, such as Trp/W or an isosterically equivalent amino acid;
X^{T7} is absent or a hydrophobic amino acid, such as Ile/I or an isosterically equivalent amino acid;
X^{T8} is absent or a hydrophobic amino acid, such as Leu/L or an isosterically equivalent amino acid;
X^{T9} is absent or a polar uncharged amino acid, such as Gln/Q or an isosterically equivalent amino acid;
X^{T10} is absent or a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I, d-Ile/dI, or an isosterically equivalent amino acid;
X^{T11} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is absent or a positively charged amino acid, such as His/H or an isosterically equivalent amino acid;
X^{T24} is a hydrophobic amino acid, such as Ala/A or an isosterically equivalent amino acid;
X^{T25} is a negatively charged amino acid, such as Glu/E or an isosterically equivalent amino acid, or
a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is absent or a hydrophobic amino acid, such as Met/M, norleucine/Nle, or an isosterically equivalent amino acid;
X^{T29} is absent or a positively charged amino acid, such as Arg/R or an isosterically equivalent amino acid;
X^{T30} is absent or a hydrophobic amino acid, such as Val/V or an isosterically equivalent amino acid, or a Cys/C or an isosterically equivalent amino acid;
X^{T31} is
Absent,
a polar uncharged amino acid, such as Ser/S or an isosterically equivalent amino acid, or
a negatively charged amino acid, such as Glu/E or an isosterically equivalent amino acid;
X^{T32} is absent or a negatively charged amino acid, such as Asp/D or an isosterically equivalent amino acid;
X^{T33} is absent or hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
absent,
a negatively charged amino acid, such as Glu/E or an isosterically equivalent amino acid, or
a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
absent,
a positively charged amino acid, such as Lys/K or an isosterically equivalent amino acid, or
a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
X^{T42} is absent or a negatively charged amino acid, such as Asp/D or an isosterically equivalent amino acid;
X^{T43} is absent or a negatively charged amino acid, such as Glu/E or an isosterically equivalent amino acid;
X^{T44} is absent or a positively charged amino acid, such as R or an isosterically equivalent amino acid;
X^{T45} is absent or a hydrophobic amino acid, such as Leu/L or an isosterically equivalent amino acid;
X^{T46} is absent or a hydrophobic amino acid, such as Leu/L or an isosterically equivalent amino acid;
X^{T47} is absent or a negatively charged amino acid, such as Glu/E or an isosterically equivalent amino acid;
X^{T48} is absent or a negatively charged amino acid, such as Glu/E or an isosterically equivalent amino acid;
X^{T49} is absent or a hydrophobic amino acid, such as Ala/A or an isosterically equivalent amino acid;
X^{T50} is absent or a negatively charged amino acid, such as Glu/E or an isosterically equivalent amino acid;
X^{T51} is absent or a positively charged amino acid, such as Arg/R or an isosterically equivalent amino acid;
X^{T52} is absent or a hydrophobic amino acid, such as Leu/L or an isosterically equivalent amino acid;
X^{T53} is absent or a hydrophobic amino acid, such as Leu/L or an isosterically equivalent amino acid;
X^{T54} is absent or a negatively charged amino acid, such as Glu/E or an isosterically equivalent amino acid;
X^{T55} is absent or a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
absent,
a positively charged amino acid, such as Arg/R or an isosterically equivalent amino acid, or
a hydrophobic amino acid, such as Ala/A, D-Ala/dA, or an isosterically equivalent amino acid;
X^{T59} is absent or a hydrophobic amino acid, such as 2-(methylamino)propanoic acid, Aib, or an isosterically equivalent amino acid;
optionally two amino acid residues of the TBM are covalently linked (e.g., two Cys/S amino acid residues form a disulfide bond) to form a cyclic structure; or
a combination thereof,
(B) an amino acid sequence X^{T1}a-X^{T64a}:
X^{T1a}X^{T2a}X^{T3a}X^{T4a}X^{T5a}X^{T6a}X^{T7a}X^{T8a}X^{T9a}X^{T10a}X^{T11a}X^{T12a}X^{T13a}X^{T14a}X^{T15a}X^{T16a}X^{T17a}X^{T118a}X^{T19a}X^{T20a}X^{T21a}X^{T22a}X^{T23a}X^{T24a}X^{T25a}X^{T26a}X^{T27a}X^{T28a}X^{T29a}X^{T30a}X^{T31a}X^{T32a}X^{T33a}X^{T34a}X^{T35a}X^{T36a}X^{T37a}X^{T38a}X^{T39a}X^{T40a}X^{T41a}X^{T42a}X^{T43a}X^{T44a}X^{T45a}X^{T46a}X^{T47a}X^{T48a}X^{T49a}X^{T50a}X^{T51a}X^{T52a}X^{T53a}X^{T54}a X^{T55a}X^{T56a}X^{T57a}X^{T58a}X^{T59a}X^{T60a}X^{T61a}X^{T62a}X^{T63a}X^{T64a}, wherein:
each of X^{T1}a through X^{T64a} is an independently selected amino acid residue), optionally the X^{T1a}-X^{T64a} sequence comprises 1, 2, 3 or 4 additional amino acids bound at any point between X^{T1}a and X^{T64a}where no more than 2 of the additional amino acid are consecutive wherein:
X^{T1a} is an uncharged, polar amino acid, such as Asn/N or an isosterically equivalent amino acid;
X^{T2a} is a negatively charged amino acid, such as Asp/D or an isosterically equivalent amino acid;
X^{T3a} is a negatively charged amino acid, such as Asp/D or an isosterically equivalent amino acid;
X^{T4a} is a negatively charged amino acid, such as Glu/E or an isosterically equivalent amino acid;
X^{T5a} is a hydrophobic amino acid, such as Leu/L or an isosterically equivalent amino acid;
X^{T6a} is a positively charged amino acid, such as His/H or an isosterically equivalent amino acid;
X^{T7a} is a hydrophobic amino acid, such as Met/M or an isosterically equivalent amino acid;
X^{T8a} is a hydrophobic amino acid, such as Leu/L or an isosterically equivalent amino acid;
X^{T9a} is a hydrophobic amino acid, such as Met/M or an isosterically equivalent amino acid;
X^{T10a} is a hydrophobic amino acid, such as Tyr/T or an isosterically equivalent amino acid;
X^{T11a} is a negatively charged amino acid, such as Asp/D or an isosterically equivalent amino acid;
X^{T12a} is a hydrophobic amino acid, such as Leu/L or an isosterically equivalent amino acid;
X^{T13a} is a hydrophobic amino acid, such as Val/V or an isosterically equivalent amino acid;
X^{T14a} is a hydrophobic amino acid, such as Tyr/Y or an isosterically equivalent amino acid;
X^{T15a} is a negatively charged amino acid, such as Glu/E or an isosterically equivalent amino acid;
X^{T16a} is a hydrophobic amino acid, such as Ala/A or an isosterically equivalent amino acid;
X^{T17a} is a hydrophobic amino acid, such as Leu/L or an isosterically equivalent amino acid;
X^{T18a} is a positively charged amino acid, such as His/H or an isosterically equivalent amino acid;
X^{T19a} is a hydrophobic amino acid, such as Phe/F or an isosterically equivalent amino acid;
X^{T20a} is a hydrophobic amino acid, such as Ala/A or an isosterically equivalent amino acid;
X^{T21a} is a positively charged amino acid, such as Lys/K or an isosterically equivalent amino acid;
X^{T22a} is a negatively charged amino acid, such as Asp/D or an isosterically equivalent amino acid;
X^{T23a} is a negatively charged amino acid, such as Glu/E or an isosterically equivalent amino acid;
X^{T24a} is a negatively charged amino acid, such as Glu/E or an isosterically equivalent amino acid;
X^{T25a} is a hydrophobic amino acid, such as Ile/I or an isosterically equivalent amino acid;
X^{T26a} is a positively charged amino acid, such as Lys/K or an isosterically equivalent amino acid;
X^{T27a} is a positively charged amino acid, such as Lys/K or an isosterically equivalent amino acid;
X^{T28a} is a positively charged amino acid, such as Arg/R or an isosterically equivalent amino acid;
X^{T29a} is a hydrophobic amino acid, such as Val/V or an isosterically equivalent amino acid;
X^{T30a} is a hydrophobic amino acid, such as Phe/F or an isosterically equivalent amino acid;
X^{T31a} is an uncharged, polar amino acid, such as Gln/Q or an isosterically equivalent amino acid;
X^{T32a} is a hydrophobic amino acid, such as Leu/L or an isosterically equivalent amino acid;
X^{T33a} is a hydrophobic amino acid, such as Phe/F or an isosterically equivalent amino acid;
X^{T34a} is a negatively charged amino acid, such as Glu/E or an isosterically equivalent amino acid;
X^{T35a} is a hydrophobic amino acid, such as Leu/L or an isosterically equivalent amino acid;
X^{T36a} is a hydrophobic amino acid, such as Ala/A or an isosterically equivalent amino acid;
X^{T37a} is a negatively charged amino acid, such as Asp/D or an isosterically equivalent amino acid;
X^{T38a} is a positively charged amino acid, such as Lys/K or an isosterically equivalent amino acid;
X^{T39a} is a hydrophobic amino acid, such as Ala/A or an isosterically equivalent amino acid;
X^{T40a} is a hydrophobic amino acid, such as Tyr/Y or an isosterically equivalent amino acid;
X^{T41a} is a positively charged amino acid, such as Lys/K or an isosterically equivalent amino acid;
X^{T42a} is an uncharged, polar amino acid, such as Asn/N or an isosterically equivalent amino acid;
X^{T43a} is an uncharged, polar amino acid, such as Asn/N or an isosterically equivalent amino acid;
X^{T44a} is a negatively charged amino acid, such as Asp/D or an isosterically equivalent amino acid;
X^{T45a} is a positively charged amino acid, such as Arg/R or an isosterically equivalent amino acid;
X^{T46a} is an uncharged, polar amino acid, such as Gln/Q or an isosterically equivalent amino acid;
X^{T47a} is a positively charged amino acid, such as Lys/K or an isosterically equivalent amino acid;
X^{T48a} is a hydrophobic amino acid, such as Leu/L or an isosterically equivalent amino acid;
X^{T49a} is a negatively charged amino acid, such as Glu/E or an isosterically equivalent amino acid;
X^{T50a} is a positively charged amino acid, such as Lys/K or an isosterically equivalent amino acid;
X^{T51a} is a hydrophobic amino acid, such as Val/V or an isosterically equivalent amino acid;
X^{T52a} is a hydrophobic amino acid, such as Val/V or an isosterically equivalent amino acid;
X^{T53a} is a negatively charged amino acid, such as Glu/E or an isosterically equivalent amino acid;
X^{T54a} is a negatively charged amino acid, such as Glu/E or an isosterically equivalent amino acid;
X^{T55a} is a hydrophobic amino acid, such as Leu/L or an isosterically equivalent amino acid;
X^{T56a} is a positively charged amino acid, such as Lys/K or an isosterically equivalent amino acid;
X^{T57a} is a negatively charged amino acid, such as Glu/E or an isosterically equivalent amino acid;
X^{T58a} is a hydrophobic amino acid, such as Leu/L or an isosterically equivalent amino acid;
X^{T59a} is a hydrophobic amino acid, such as Leu/L or an isosterically equivalent amino acid;
X^{T60a} is a negatively charged amino acid, such as Glu/E or an isosterically equivalent amino acid;
X^{T61a} is a positively charged amino acid, such as Arg/R or an isosterically equivalent amino acid;
X^{T62a} is a hydrophobic amino acid, such as Leu/L or an isosterically equivalent amino acid;
X^{T63a} is a hydrophobic amino acid, such as Leu/L or an isosterically equivalent amino acid;
X^{T64a} is an uncharged, polar amino acid, such as Ser/S or an isosterically equivalent amino acid; or a combination thereof, or
(C) an amino acid sequence X^{T1}-X^{T40}:
X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40} wherein:
each of X^{T1} through X^{T40} is an independently selected amino acid residue;
two amino acid residues of the TBM are covalently linked (e.g., two Cys/S amino acid residues form a disulfide bond) to form a cyclic structure; and
either X^{T15} and X^{T30} are Cys/S covalently linked with a disulfide bond or X^{T18} and X^{T27} are Cys/S covalently linked with a disulfide bond;
optionally one or more of X^{T1} to X^{T8} are absent, and
optionally the X^{T1}-X^{T40} sequence comprises 1, 2, 3 or 4 additional amino acids bound at any point between X^{T1} and X^{T40} where no more than 2 of the additional amino acid are consecutive,
wherein:
X^{T1} is absent or a hydrophobic amino acid, such as Ala/A, Aib, or an isosterically equivalent amino acid;
X^{T2} is absent or a polar uncharged amino acid, such as Ser/S or an isosterically equivalent amino acid;
X^{T3} is
absent,
a negatively charged amino acid, such as Asp/D, D-Asp/dD, or an isosterically equivalent amino acid, or
a hydrophobic amino acid, such as Ala/A, D-Ala/dA, or an isosterically equivalent amino acid;
X^{T4} is absent or a positively charged amino acid, such as Lys/K or an isosterically equivalent amino acid;
X^{T5} is absent or a negatively charges amino acid, such as Glu/E or an isosterically equivalent amino acid;
X^{T6} is absent or a hydrophobic amino acid, such as Trp/W or an isosterically equivalent amino acid;
X^{T7} is absent or a hydrophobic amino acid, such as Ile/I or an isosterically equivalent amino acid;
X^{T8} is absent or a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C or an isosterically equivalent amino acid, which Cys/C is bound via a disulfide bond to X^{T30};
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid, which Cys/S is bound via a disulfide bond to X^{T27};
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is a negatively charged amino acid, such as Glu/E or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C which is bound to X^{T18} via a disulfide bond;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T50} is a hydrophobic amino acid, such as Val/V or an isosterically equivalent amino acid, or a Cys/C which is bound to X^{T15} via a disulfide bound;
X^{T31} is a polar uncharged amino acid, such as Ser/S or an isosterically equivalent amino acid;
X^{T132} is a negatively charged amino acid, such as Asp/D or an isosterically equivalent amino acid;
X^{T33} is a hydrophobic amino acid, such as Leu/L or an isosterically equivalent amino acid;
X^{T34} is a hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is absent or a hydrophobic amino acid, such as Phe/F or an isosterically equivalent amino acid;
X^{T38} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
absent,
a positively charged amino acid, such as Lys/K or an isosterically equivalent amino acid, or
a hydrophobic amino acid, such as Aib, Ala, or a sterically equivalent amino acid.

6. The compound of any one of claims 1-6, or a pharmaceutically acceptable salt thereof, wherein each TBM independently is or includes
(a) X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40} X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
X^{T1} is absent or a hydrophobic amino acid, such as Ala/A, Aib, or an isosterically equivalent amino acid;
X^{T2} is absent or a polar uncharged amino acid, such as Ser/S or an isosterically equivalent amino acid;
X^{T3} is
a negatively charged amino acid, such as Asp/D, D-Asp/dD, or an isosterically equivalent amino acid, or
a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I, d-Ile/dI or an isosterically equivalent amino acid;
X^{T11} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or
a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C or an isosterically equivalent amino acid;
X^{T31} is
a polar uncharged amino acid, such as Ser/S or an isosterically equivalent amino acid, or
a negatively charged amino acid, such as Glu/E or an isosterically equivalent amino acid;
X^{T132} is a negatively charged amino acid, such as Asp/D or an isosterically equivalent amino acid;
X^{T33} is a hydrophobic amino acid, such as Leu/L or an isosterically equivalent amino acid;
X^{T34} is a hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is a hydrophobic amino acid, such as Phe/F or an isosterically equivalent amino acid;
X^{T31} is a hydrophobic amino acid, such as Met/M, norleucine/Nle, or an isosterically equivalent amino acid;
X^{T39} is a positively charged amino acid, such as Lys/K or an isosterically equivalent amino acid;
X^{T40} is
a positively charged amino acid, such as Lys/K or an isosterically equivalent amino acid, or
a hydrophobic amino acid, such as Aib, Ala, or a sterically equivalent amino acid;
X^{T41} is an uncharged, non-polar amino acid, such as Gly/G, Ala/A, D-Ala/dA, Phe/F, D-Phe/dF, or an isosterically equivalent amino acid;
X^{T42} is a negatively charged amino acid, such as Asp/D or an isosterically equivalent amino acid;
X^{T43} is a negatively charged amino acid, such as Glu/E or an isosterically equivalent amino acid;
X^{T44} is a positively charged amino acid, such as R or an isosterically equivalent amino acid;
X^{T45} is a hydrophobic amino acid, such as Leu/L or an isosterically equivalent amino acid;
X^{T46} is a hydrophobic amino acid, such as Leu/L or an isosterically equivalent amino acid;
X^{T47} is a negatively charged amino acid, such as Glu/E or an isosterically equivalent amino acid;
X^{T48} is a negatively charged amino acid, such as Glu/E or an isosterically equivalent amino acid;
X^{T49} is a hydrophobic amino acid, such as Ala/A or an isosterically equivalent amino acid;
X^{T10} is a negatively charged amino acid, such as Glu/E or an isosterically equivalent amino acid;
X^{T11} is a positively charged amino acid, such as Arg/R or an isosterically equivalent amino acid;
X^{T52} is a hydrophobic amino acid, such as Leu/L or an isosterically equivalent amino acid;
X^{T53} is a hydrophobic amino acid, such as Leu/L or an isosterically equivalent amino acid;
X^{T54} is a negatively charged amino acid, such as Glu/E or an isosterically equivalent amino acid;
X^{T55} is a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
absent,
a positively charged amino acid, such as Arg/R or an isosterically equivalent amino acid, or
a hydrophobic amino acid, such as Ala/A, D-Ala/dA, or an isosterically equivalent amino acid;
X^{T59} is absent or a hydrophobic amino acid, such as 2-(methylamino)propanoic acid, Aib, or an isosterically equivalent amino acid;
(b) X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}, wherein:
at least two amino acid residues of the TBM are covalently linked (e.g., two Cys/S amino acid residues form a disulfide bond) to form a cyclic structure,
X^{T15} and X^{T30} are Cys/S covalently linked with a disulfide bond and/or X^{T18} and X^{T27} are Cys/S covalently linked with a disulfide bond:
X^{T1} is absent or a hydrophobic amino acid, such as Ala/A, Aib, or an isosterically equivalent amino acid;
X^{T2} is absent or a polar uncharged amino acid, such as Ser/S or an isosterically equivalent amino acid;
X^{T3} is
a negatively charged amino acid, such as Asp/D, D-Asp/dD, or an isosterically equivalent amino acid, or
a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C, which Cys/C can be bound via a disulfide bond to X^{T30};
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C, which Cys/S can be bound via a disulfide bond to X^{T27};
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C, which can be bound to X^{T18} via a disulfide bond;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C, which can be bound to X^{T15} via a disulfide bound;
X^{T31} is
a polar uncharged amino acid, such as Ser/S or an isosterically equivalent amino acid, or
a negatively charged amino acid, such as Glu/E or an isosterically equivalent amino acid;
X^{T32} is a negatively charged amino acid, such as Asp/D or an isosterically equivalent amino acid;
X^{T33} is a hydrophobic amino acid, such as Leu/L or an isosterically equivalent amino acid;
X^{T34} is a hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is a hydrophobic amino acid, such as Phe/F or an isosterically equivalent amino acid;
X^{T3}8 is a hydrophobic amino acid, such as Met/M, norleucine/Nle, or an isosterically equivalent amino acid;
X^{T39} is a positively charged amino acid, such as Lys/K or an isosterically equivalent amino acid;
X^{T40} is
a positively charged amino acid, such as Lys/K or an isosterically equivalent amino acid, or
a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
(c) I X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40} X^{T41}X^{T42}X^{T43}X^{T44}X^{T45}X^{T46}X^{T47}X^{T48}X^{T49}X^{T50}X^{T51}X^{T52}X^{T53}X^{T54}X^{T55}X^{T56}X^{T57}X^{T58}X^{T59}, wherein:
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or
a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C or an isosterically equivalent amino acid;
X^{T31} is
a polar uncharged amino acid, such as Ser/S or an isosterically equivalent amino acid, or
a negatively charged amino acid, such as Glu/E or an isosterically equivalent amino acid;
X^{T32} is a negatively charged amino acid, such as Asp/D or an isosterically equivalent amino acid;
X^{T33} is hydrophobic amino acid, such as Leu/L or an isosterically equivalent amino acid;
X^{T34} is hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is a hydrophobic amino acid, such as Phe/F or an isosterically equivalent amino acid;
X^{T31} is a hydrophobic amino acid, such as Met/M, norleucine/Nle, or an isosterically equivalent amino acid;
X^{T39} is a positively charged amino acid, such as Lys/K or an isosterically equivalent amino acid;
X^{T40} is
a positively charged amino acid, such as Lys/K or an isosterically equivalent amino acid, or
a hydrophobic amino acid, such as Aib, Ala, or a sterically equivalent amino acid;
X^{T41} is an uncharged, non-polar amino acid, such as Gly/G, Ala/A, D-Ala/dA, Phe/F, D-Phe/dF, or an isosterically equivalent amino acid;
X^{T42} is a negatively charged amino acid, such as Asp/D or an isosterically equivalent amino acid;
X^{T43} is a negatively charged amino acid, such as Glu/E or an isosterically equivalent amino acid;
X^{T44} is a positively charged amino acid, such as R or an isosterically equivalent amino acid;
X^{T45} is a hydrophobic amino acid, such as Leu/L or an isosterically equivalent amino acid;
X^{T46} is a hydrophobic amino acid, such as Leu/L or an isosterically equivalent amino acid;
X^{T47} is a negatively charged amino acid, such as Glu/E or an isosterically equivalent amino acid;
X^{T48} is a negatively charged amino acid, such as Glu/E or an isosterically equivalent amino acid;
X^{T49} is a hydrophobic amino acid, such as Ala/A or an isosterically equivalent amino acid;
X^{T10} is a negatively charged amino acid, such as Glu/E or an isosterically equivalent amino acid;
X^{T51} is a positively charged amino acid, such as Arg/R or an isosterically equivalent amino acid;
X^{T52} is a hydrophobic amino acid, such as Leu/L or an isosterically equivalent amino acid;
X^{T53} is a hydrophobic amino acid, such as Leu/L or an isosterically equivalent amino acid;
X^{T54} is a negatively charged amino acid, such as Glu/E or an isosterically equivalent amino acid;
X^{T55} is a negatively charged amino acid, such as Glu/E or an isosterically equivalent amino acid;
X^{T56} is absent or a hydrophobic amino acid, such as Val/V or an isosterically equivalent amino acid;
X^{T57} is absent or a negatively charged amino acid, such as Glu/E or an isosterically equivalent amino acid;
X^{T58} is
absent,
a positively charged amino acid, such as Arg/R or an isosterically equivalent amino acid, or
a hydrophobic amino acid, such as Ala/A, D-Ala/dA, or an isosterically equivalent amino acid;
X^{T59} is absent or a hydrophobic amino acid, such as 2-(methylamino)propanoic acid, Ala/A, or an isosterically equivalent amino acid;
(d) X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41}, wherein:
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C or an isosterically equivalent amino acid;
X^{T31} is
a polar uncharged amino acid, such as Ser/S or an isosterically equivalent amino acid, or
a negatively charged amino acid, such as Glu/E or an isosterically equivalent amino acid;
X^{T32} is a negatively charged amino acid, such as Asp/D or an isosterically equivalent amino acid;
X^{T33} is a hydrophobic amino acid, such as Leu/L or an isosterically equivalent amino acid;
X^{T34} is a hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is a hydrophobic amino acid, such as Phe/F or an isosterically equivalent amino acid;
X^{T31} is a hydrophobic amino acid, such as Met/M, norleucine/Nle, or an isosterically equivalent amino acid;
X^{T39} is a positively charged amino acid, such as Lys/K or an isosterically equivalent amino acid;
X^{T40} is
a positively charged amino acid, such as Lys/K or an isosterically equivalent amino acid, or
a hydrophobic amino acid, such as Aib, Ala, or a sterically equivalent amino acid;
X^{T41} is an uncharged, non-polar amino acid, such as Gly/G, Ala/A, D-Ala/dA, Phe/F, D-Phe/dF, or an isosterically equivalent amino acid;
(e)X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41} wherein:
X^{T1} is absent or a hydrophobic amino acid, such as Ala/A, Aib, or an isosterically equivalent amino acid;
X^{T2} is absent or a polar uncharged amino acid, such as Ser/S or an isosterically equivalent amino acid;
X^{T3} is
a negatively charged amino acid, such as Asp/D, D-Asp/dD, or an isosterically equivalent amino acid, or
a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid, or a Cys/C or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or
a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid, or a Cys/C or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid;
X^{T31} is
a polar uncharged amino acid, such as Ser/S or an isosterically equivalent amino acid, or
a negatively charged amino acid, such as Glu/E or an isosterically equivalent amino acid;
X^{T32} is a negatively charged amino acid, such as Asp/D or an isosterically equivalent amino acid;
X^{T33} is a hydrophobic amino acid, such as Leu/L or an isosterically equivalent amino acid;
X^{T34} is a hydrophobic amino acid, such as Ile/I or an isosterically equivalent amino acid;
X^{T35} is a hydrophobic amino acid, such as Tyr/Y or an isosterically equivalent amino acid;
X^{T36} is
a negatively charged amino acid, such as Glu/E or an isosterically equivalent amino acid, or
a hydrophobic amino acid, such as Aib, Ala/A, or an isosterically equivalent amino acid;
X^{T37} is a hydrophobic amino acid, such as Phe/F or an isosterically equivalent amino acid;
X^{T38} is a hydrophobic amino acid, such as Met/M, norleucine/Nle, or an isosterically equivalent amino acid;
X^{T39} is a positively charged amino acid, such as Lys/K or an isosterically equivalent amino acid;
X^{T40} is
a positively charged amino acid, such as Lys/K or an isosterically equivalent amino acid, or
a hydrophobic amino acid, such as Aib, Ala, or a sterically equivalent amino acid;
X^{T41} is an uncharged, non-polar amino acid, such as Gly/G, Ala/A, D-Ala/dA, Phe/F, D-Phe/dF, or an isosterically equivalent amino acid;
optionally X^{T18} and X^{T27} form a disulfide bond; or
a combination thereof, or
(f)X^{T1}X^{T2}X^{T3}X^{T4}X^{T5}X^{T6}X^{T7}X^{T8}X^{T9}X^{T10}X^{T11}X^{T12}X^{T13}X^{T14}X^{T15}X^{T16}X^{T17}X^{T18}X^{T19}X^{T20}X^{T21}X^{T22}X^{T23}X^{T24}X^{T25}X^{T26}X^{T27}X^{T28}X^{T29}X^{T30}X^{T31}X^{T32}X^{T33}X^{T34}X^{T35}X^{T36}X^{T37}X^{T38}X^{T39}X^{T40}X^{T41} wherein:
X^{T1} is absent or a hydrophobic amino acid, such as Ala/A, Aib, or an isosterically equivalent amino acid;
X^{T2} is absent or a polar uncharged amino acid, such as Ser/S or an isosterically equivalent amino acid;
X^{T3} is
a negatively charged amino acid, such as Asp/D, D-Asp/dD, or an isosterically equivalent amino acid, or
a hydrophobic amino acid, such as Ala/A, D-Ala/dA, or an isosterically equivalent amino acid;
X^{T4} is a positively charged amino acid, such as Lys/K or an isosterically equivalent amino acid;
X^{T5} is a negatively charges amino acid, such as Glu/E or an isosterically equivalent amino acid;
X^{T6} is a hydrophobic amino acid, such as Trp/W or an isosterically equivalent amino acid;
X^{T7} is a hydrophobic amino acid, such as Ile/I or an isosterically equivalent amino acid;
X^{T8} is a hydrophobic amino acid, such as Leu/L or an isosterically equivalent amino acid;
X^{T9} is a polar uncharged amino acid, such as Gln/Q or an isosterically equivalent amino acid;
X^{T10} is a positively charged amino acid, such as Lys/K or an isosterically equivalent amino acid;
X^{T11} is a hydrophobic amino acid, such as Ile/I or an isosterically equivalent amino acid;
X^{T12} is a hydrophobic amino acid, such as Tyr/Y or an isosterically equivalent amino acid;
X^{T13} is a negatively charged amino acid, such as Glu/E or an isosterically equivalent amino acid;
X^{T14} is a hydrophobic amino acid, such as Ile/I or an isosterically equivalent amino acid;
X^{T15} is a hydrophobic amino acid, such as Met/M, norleucine/Nle, or an isosterically equivalent amino acid, or a Cys/C or an isosterically equivalent amino acid;
X^{T16} is a positively charged amino acid, such as Arg/R or an isosterically equivalent amino acid;
X^{T17} is a hydrophobic amino acid, such as Leu/L or an isosterically equivalent amino acid;
X^{T18} is a hydrophobic amino acid, such as Leu/L or an isosterically equivalent amino acid;
X^{T19} is a negatively charged amino acid, such as Asp/D or an isosterically equivalent amino acid;
X^{T20} is a negatively charged amino acid, such as Glu/E or an isosterically equivalent amino acid;
X^{T21} is a hydrophobic amino acid, such as Leu/L, norleucine/Nle, or an isosterically equivalent amino acid;
X^{T22} is
an uncharged, non-polar amino acid, such as Gly/G or an isosterically equivalent amino acid, or
a positively charged amino acid, such as His/H, D-His/dH, or an isosterically equivalent amino acid;
X^{T23} is a positively charged amino acid, such as His/H or an isosterically equivalent amino acid;
X^{T24} is a hydrophobic amino acid, such as Ala/A or an isosterically equivalent amino acid;
X^{T25} is
a negatively charged amino acid, such as Glu/E or an isosterically equivalent amino acid, or
a Pro/P or an isosterically equivalent amino acid;
X^{T26} is a hydrophobic amino acid, such as Ala/A or an isosterically equivalent amino acid;
X^{T27} is a polar uncharged amino acid, such as Ser/S or an isosterically equivalent amino acid;
X^{T28} is a hydrophobic amino acid, such as Met/M, norleucine/Nle, or an isosterically equivalent amino acid;
X^{T29} is a positively charged amino acid, such as Arg/R or an isosterically equivalent amino acid;
X^{T30} is a hydrophobic amino acid, such as Val/V or an isosterically equivalent amino acid, or a Cys/C or an isosterically equivalent amino acid;
X^{T31} is
a polar uncharged amino acid, such as Ser/S or an isosterically equivalent amino acid, or
a negatively charged amino acid, such as Glu/E or an isosterically equivalent amino acid;
X^{T32} is a negatively charged amino acid, such as Asp/D or an isosterically equivalent amino acid;
X^{T33} is absent or a hydrophobic amino acid, such as Leu/L or an isosterically equivalent amino acid;
X^{T34} is absent or a hydrophobic amino acid, such as Ile/I or an isosterically equivalent amino acid;
X^{T35} is absent or a hydrophobic amino acid, such as Tyr/Y or an isosterically equivalent amino acid;
X^{T36} is
absent,
a negatively charged amino acid, such as Glu/E or an isosterically equivalent amino acid, or
a hydrophobic amino acid, such as Aib, Ala/A, or an isosterically equivalent amino acid;
X^{T37} is absent or a hydrophobic amino acid, such as Phe/F or an isosterically equivalent amino acid;
X^{T31} is absent or a hydrophobic amino acid, such as Met/M, norleucine/Nle, or an isosterically equivalent amino acid;
X^{T39} is absent or a positively charged amino acid, such as Lys/K or an isosterically equivalent amino acid;
X^{T40} is
absent,
a positively charged amino acid, such as Lys/K or an isosterically equivalent amino acid, or
a hydrophobic amino acid, such as Aib, Ala, or a sterically equivalent amino acid;
X^{T41} is absent or an uncharged, non-polar amino acid, such as Gly/G, Ala/A, D-Ala/dA, Phe/F, D-Phe/dF, or an isosterically equivalent amino acid;
optionally X^{T15} and X^{T30} form a disulfide bond; or
a combination thereof.

7. The compound of any one of claims 1-7, wherein one or more TBM, independently, is or comprises a TBM from an exemplary compound of the present disclosure, such as an optionally substituted amino acid sequence independently selected from:
DKEWILQKIYEIMRLLDELGHAEASMRVSDLIYEFMKKGDERLLEEAERLLEEVER,
DCAWILQKIYEIMRLLDELGHAEASMRVSDLIYEFMKKGDERLLEEAERLLEEVER,
DKEWILQKIYEIMRLLDELGHAPASMRVSDLIYEFMKKGDERLLEEAERLLEEVER,
DKEWILQKIYEIMRLLDELGHAEASMRVSDLIYEFMKKG,
DKEWILQKIYEIMRLLDELGHAEASMRVSDLIYEFMKK,
DKEWILQKIYEIMRLLDELGHAEASMRVSDLIYE,
DKEWILQKIYEIMRLLDELGHAPASMRVER,
DKEWILQKIYEIMRLLDELGHAEASMRVS,
DKEWILQKIYEIMRLLDELGHAEAS,
DKEWILQKIYEIMRLLDELG,
ILQKIYEIMRLLDELGHAEASMRVSDLIYEFMKKG,
IYEIMRLLDELGHAEASMRVSDLIYEFMKKG,
ILQKIYEIMRLLDELGHAEASMRVSDLIYEFMKK,
[Aib]DKEWILQKIYEIMRLLDELGHAEASMRVSDLIYEFMKKG,
[Aib]SDKEWILQKIYEIMRLLDELGHAEASMRVSDLIYEFMKK[dA],
[Aib]SDKEWILQKIYEIMRLLDELGHAEASMRVSDLIYEFMKK[dF],
D[Abu]EWILQKIYEIMRLLDELG,
[Aib]SDKEWILQKIYEI[Nle]RLLDELGHAE,
[Aib]SDKEWILQLIYEIMRLLDELGHAEASMRVSDLIYEFMKK[dA],
[Aib]SDKEWILQKIYEIMRLLDELGHAEASMRVSDLIYEFMKK[dF],
[Aib]SDKEWILQKIYEIMRLLDELGHAEAS[Nle]RVSDLIY[Aib]FMK[Aib]G,
DKEWILQKIYEIMRLLDEL[DHis]HAPASMRVSDLIYEFMKKGDERLLEEAERLLEEVER,
DKEWILQLIYEI[Nle]RLLDELGHAEASMRVSDLIYEF[Nle]KKGDERLLEEAERLLEEVER,
DKEWILQKIYEIMRLLDE[Nle][DHis]HAEASMRVSDLIYEFMKKGDERLLEEAERLLEEVER,
DKEWILQKIYEIMRLLDE[Nle][DHis]HAEASMRVSDLIYEFMKKG,
DKEWILQKIYE[d]]MRLLDELGHAEASMRVSDLIYEFMKKGDERLLEEAERLLEEVER,
DKEWILQKIYEIMRLLDEL[DHis]HAPASM RVSDLIYEFMKKGDERLLEEAERLLEEVER,
[Aib]SDKEWILQKIYEI[Nle]RLLDELGHAEASM
RVSDLIYEHMKKGDERLLEEAERLLE[Aib]VER[Aib],
[Aib]SDKEWILQKIYEIMRLLDELGHAEAS[Nle]RVSDLIY[Aib]FMK[Aib]G,
[Aib]SDKEWILQKIYEI[Nle]RLLDELGHAEAS[Nle]RVSDLIY[Aib]FMK[Aib]G,
SDKEWILQKIYEIMRLLDELGHAPASMRVSDLIYEFMKKG,
DKEWILQKIYEIMRLLDELGHAEASMRVSDLIYEFMKKGDERLLEEAERLLEEVE[DAla],
DKEWILQKIYEIMRLLDELGHAPASMRVSDLIYEFMKKGDERLLEEAERLLEEVE[DAla],
DKEWILQKIYEIMRLLDELGHAEASMRVSDLIYEFMKKGDERLLEEAERLLEEVEA[2-
(methylamino)propanoic acid],
[DAla]KEWILQKIYEIMRLLDELGHAPASMRVSDLIYEFMKKGDERLLEEAERLLEEVER,
SDKEWILQKIYEIMRL-Cyclo[CDELGHAEAC]MRVSDLIYEFMKKG,
DKEWILQKIYEIMRL-Cyclo[CDELGHAEAC]-MRVSDLIYEFMKKG
ILQKIYEIMRL-Cyclo[CDELGHAEAC]-MRVSDLIYEFMKKG,
QKIYEIMRL-Cyclo[CDELGHAEAC]-MRVSDLIYEFMKKG,
DKEWILQKIYEIMRL-Cyclo[CDELGHAEAC]MRVSDLIYEFMKK,
DKEWILQKIYEI-Cyclo[CRLLDELGHAEASMRC]-SDLIYEFMKKG,
ILQKIYEI-Cyclo[CRLLDELGHAEASMRC]-SDLIYEFMKKG,
or
QKIYEI-Cyclo[CRLLDELGHAEASMRC]-SDLIYEFMKKG.

8. The compound of any one of claims 1-8, or a pharmaceutically acceptable salt thereof, wherein:
the ABM is a universal antibody binding moiety;
the ABM can bind to a Fab region of an antibody, a Fc region of an antibody, a Fc region bound to an Fc receptor, or two or more antibodies (e.g., 2, 3, 4, or more antibodies) that have different Fab regions; or
a combination thereof.

9. The compound of any one of claims 1-9, or a pharmaceutically acceptable salt thereof, wherein each ABM or universal antibody binding moiety is independently selected from: 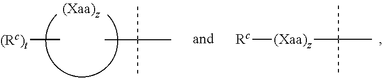
wherein:
each Xaa is an independently selected amino acid residue (such as, a natural/proteinogenic amino acid, a unnatural/non-proteinogenic amino acid, or a combination thereof);
t is an integer from 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, or 50);
z is an integer from 3 to 50 (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 59, or 50);
each R^{c} is independently -L^{a}-R′;
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₁₋₃₀ arylaliphatic, C₁₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

10. The compound of any one of claims 1-10, wherein:
(a) one or more -(Xaa)z-, independently, is or comprises [X¹]ₚ₁[X²]ₚ₂-X³X⁴X⁵X⁶X⁷X⁸X⁹X¹⁰X¹¹X¹²-[X¹³]ₚ₁₃[X¹⁴]ₚ₁₄[X¹⁵]ₚ₁₅[X¹⁶]ₚ₁₆,
wherein:
each of X¹, X², X³, X⁴, X⁵, X⁶, X⁷, X⁸, X⁹, X¹⁰, X¹¹, X¹², and X¹³ is an independently selected amino acid residue (e.g., of an amino acid of formula NH(R^{a1})-L^{a1}-C(R^{a2})(R^{a3})-L^{a2}-COOH), and
each of p1, p2, p13, p14, p15 and p16 is independently 0, 1, 2, 3, 4, 5, 6, 7, 8, 9, or 10;
(b) one or more -(Xaa)z-, independently, is or comprises —X³X⁴X⁵X⁶X⁷X⁸X⁹X¹⁰X¹¹X¹²-, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P};
(c) one or more -(Xaa)z-, independently, is or comprises —X³X⁴X⁵X⁶X⁷X⁸X⁹X¹⁰X¹¹X¹²-, wherein:
each of X³, X⁴, X⁵, X⁶, X⁷, X⁸, X⁹X¹⁰, X¹¹, and X¹² is an independently selected amino acid residue;
at least two amino acid residues are connected through one or more linkages of L;
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P};
(d) one or more -(Xaa)z-, independently, is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹, X¹¹, and X¹² is an independently selected amino acid residue;
at least two amino acid residues are connected through one or more linkages of L;
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A};
(e) one or more -(Xaa)z-, independently, is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹, X¹¹, and X¹² is an independently selected amino acid residue;
at least two amino acid residues are connected through one or more linkages of L;
L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A};
(f) one or more, -(Xaa)z-, independently, is or comprises —X²X³X⁴X⁵X⁶X⁷X⁸X⁹X¹⁰X¹¹X¹²X¹³X¹⁴X¹⁵X¹⁶-, wherein:
each of X², X³, X⁴, X⁵, X⁶, X⁷, X⁸, X⁹, X¹⁰, X¹¹, X¹², X¹³, X¹⁴, X¹⁵, and X¹⁶ is an independently selected amino acid residue;
at least two amino acid residues are connected through a linkage of L^{b};
L^{b} is an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}; or
(g) a combination thereof,
wherein
Xaa^{A} is an amino acid residue (e.g., a residue of an amino acid of formula NH(R^{a1})-L^{a1}-C(R^{a2})(R^{a3})-L^{a2}-COOH) that has a side chain comprising an aromatic group (“aromatic amino acid residue”);
Xaa^{P} is an amino acid residue (e.g., a residue of an amino acid of formula NH(R^{a1})-L^{a1}-C(R^{a2})(R^{a3})-L^{a2}-COOH) that has a positively charged side chain (e.g., at physiological pH of about 7.4, “positively charged amino acid residue”); and
Xaa^{N} is an amino acid residue (e.g., a residue of an amino acid of formula NH(R^{a1})-L^{a1}-C(R^{a2})(R^{a3})-L^{a2}-COOH) that has a negatively charged side chain (e.g., at physiological pH about 7.4, “negatively charged amino acid residue”).

11. The compound of claim 11, wherein (b) one or more -(Xaa)z-, independently, is or comprises —X³X⁴X⁵X⁶X⁷X⁸X⁹X¹⁰X¹¹X¹²-, wherein:
each of X³, X⁴, X⁵, X⁶, X⁷, X⁸, X⁹, X¹⁰, X¹¹, and X¹² is an independently an amino acid of formula NH(R^{a1})-L^{a1}-C(R^{a2})(R^{a3})-L^{a2}-COOH as described in the present disclosure;
X³ is Lys;
X⁴ is Pro;
X⁵ is Xaa^{A} or Xaa^{P}, such as an amino acid residue whose side chain comprises an optionally substituted saturated, partially saturated or aromatic ring (e.g., 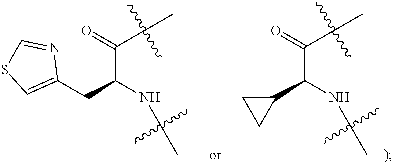 [SMILES: *N[C@@H](Cc1cscn1)C(*)=O ; *N[C@H](C(*)=O)C1CC1]
X⁶ is His;
X⁸ is Gly;
X⁹ is Asp or Glu;
each of X⁷, X¹⁰, and X¹¹ is independently an amino acid residue with a hydrophobic side chain (“hydrophobic amino acid residue”, Xaa^{H}) (such as X⁷ is 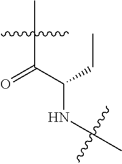 [SMILES: *N[C@@H](CC)C(*)=O]
or Val, X¹⁰ is Met or 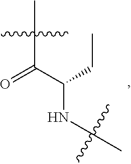 [SMILES: *N[C@@H](CC)C(*)=O]
X¹¹ is 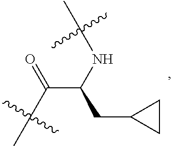 [SMILES: *N[C@@H](CC1CC1)C(*)=O]
or a combination thereof);
X¹² is 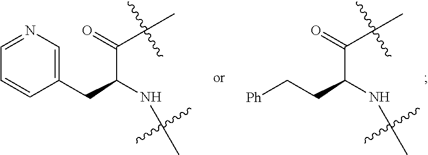 [SMILES: *N[C@@H](Cc1cccnc1)C(*)=O ; *N[C@@H](CCc1ccccc1)C(*)=O]
optionally the —COOH of X¹² forms an amide bond with the side chain amino group of Lys (X³), and the other amino group of the Lys (X³) is connected to a linker moiety and then a target binding moiety; or
a combination thereof.

12. The compound of claim 11, wherein (c) one or more -(Xaa)z-, independently, is or comprises —X³X⁴X⁵X⁶X⁷X⁸X⁹X¹⁰X¹¹X¹²-, wherein:
each of X³, X⁴, X⁵, X⁶, X⁷, X⁸, X⁹, X¹⁰, X¹¹, and X¹² is an independently an amino acid of formula NH(R^{a1})-L^{a1}-C(R^{a2})(R^{a3})-L^{a2}-COOH as described in the present disclosure;
two non-neighboring amino acid residues are connected by L^{b}, such as X⁵ and X¹⁰ are connected by L^{b};
X³ is Lys;
X⁶ is His, Asp, or Glu;
X¹² is Xaa^{A}, such as 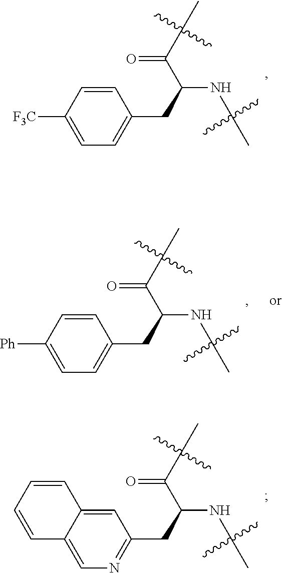 [SMILES: *N[C@@H](Cc1ccc(C(F)(F)F)cc1)C(*)=O ; *N[C@@H](Cc1ccc(-c2ccccc2)cc1)C(*)=O ; *N[C@@H](Cc1cc2ccccc2cn1)C(*)=O]
each of X⁴, X⁷, and X¹¹ is independently selected from Xaa^{H} (such as, X⁴ is Ala, X⁷ is 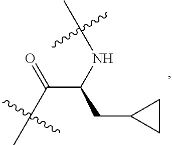 [SMILES: *N[C@@H](CC1CC1)C(*)=O]
X¹¹ is 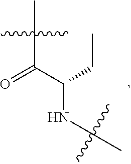 [SMILES: *N[C@@H](CC)C(*)=O]
or a combination thereof);
X⁸ is Gly;
optionally the —COOH of X¹² forms an amide bond with the side chain amino group of Lys (X³), and the other amino group of the Lys (X³) is connected to a linker moiety and then a target binding moiety; or
a combination thereof.

13. The compound of claim 13, wherein
L^{b} is an optionally substituted 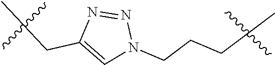 [SMILES: *CCCn1cc(C*)nn1]
or an optionally substituted 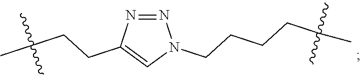 [SMILES: *CCCCn1cc(CC*)nn1]
L^{b} connects two alpha-carbon atoms of two different amino acid residues;
both X⁵ and X¹⁰ are Cys, and the two —SH groups of their side chains form —S—S— (e.g., L^{b} is —CH₂—S—S—CH₂—); or
a combination thereof.

14. The compound of claim 11, wherein (d) one or more -(Xaa)z-, independently, is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰, X¹¹, and X¹² is an independently selected amino acid of formula NH(R^{a1})-L^{a1}-C(R^{a2})(R^{a3})-L^{a2}-COOH as described in the present disclosure;
two non-neighboring amino acid residues are connected by L^{b};
L^{b} connects two alpha-carbon atoms of two different amino acid residues;
there is one linkage L^{b};
X² and X¹² are connected by L^{b};
L^{b} is an optionally substituted —CH₂—S—S—CH₂—, an optionally substituted —CH₂—CH₂—S—CH₂-; an optionally substituted 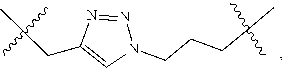 [SMILES: *CCCn1cc(C*)nn1]
in optionally substituted 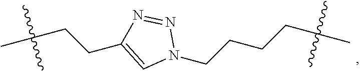 [SMILES: *CCCCn1cc(CC*)nn1]
an optionally substituted 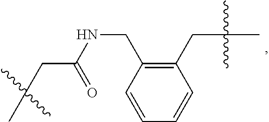 [SMILES: *CC(=O)NCc1ccccc1C*]
or an optionally substituted —CH₂CH₂CO—N(R′)—CH₂CH₂—, wherein R′ is optionally taken together with an R group on the backbone atom that —N(R′)—CH₂CH₂— is bonded to form a ring (e.g., a formed ring is a 3-, 4-, 5-, 6-, 7- or 8-membered ring, such as a monocyclic ring, a saturated ring, or both); or
a combination thereof.

15. The compound of claim 11, wherein (e) one or more -(Xaa)z-, independently, is or comprises —X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰X¹¹, and X¹² is an independently selected amino acid of formula NH(R^{a1})-L^{a}—C(R^{a2})(R^{a3})-L^{a2}-COOH as described in the present disclosure;
two non-neighboring amino acid residues are connected by L^{b} (e.g., L^{b} connects two alpha-carbon atoms of two different amino acid residues);
X² and X¹² are connected by L^{b} (such as, L^{b} is —CH₂—S—CH₂CH₂— or both X² and X¹² are Cys, and the two —SH groups of their side chains form —S—S—, for example L^{b} is —CH₂—S—S—CH₂—);
X⁴ and X⁹ are connected by L^{b} (such as X⁴ and X⁹ are connected by L^{b}, wherein L^{b} is an optionally substituted 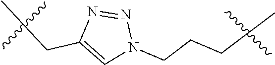 [SMILES: *CCCn1cc(C*)nn1]
or an optionally substituted 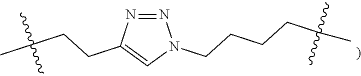 [SMILES: *CCCCn1cc(CC*)nn1]
or X⁴ and X¹⁰ are connected by L^{b} (such as both X⁴ and X¹⁰ are Cys, and the two —SH groups of their side chains form —S—S— (e.g., L^{b} is —CH₂—S—S—CH₂—));
L^{b} is —CH₂—S—S—CH₂—, an optionally substituted 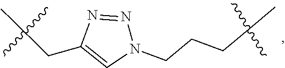 [SMILES: *CCCn1cc(C*)nn1]
an optionally substituted 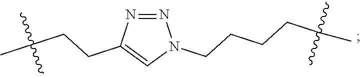 [SMILES: *CCCCn1cc(CC*)nn1]
X¹ is Xaa^{N}, such as Asp or Glu;
X⁵ is His;
X⁷ is Gly;
X⁸ is Asp or Glu;
X¹¹ is Tyr, an optionally substituted 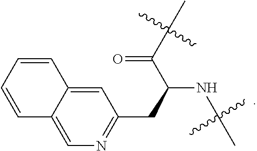 [SMILES: *N[C@@H](Cc1cc2ccccc2cn1)C(*)=O]
one or more of X³, X⁶, and X⁹ is independently selected from Xaa^{H} (such as X³ is Ala, X⁶ is Leu or 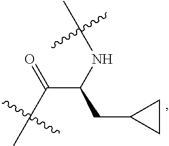 [SMILES: *N[C@@H](CC1CC1)C(*)=O]
X⁹ is Leu or 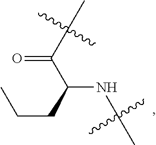 [SMILES: *N[C@@H](CCC)C(*)=O]
or a combination thereof);
X¹⁰ is Xaa^{H}, such as Val; or
a combination thereof.

16. The compound of claim 11, wherein (f) one or more, -(Xaa)z-, independently, is or comprises —X²X³X⁴X⁵X⁶X⁷X⁸X⁹X¹⁰X¹¹X¹²X¹³X¹⁴X¹⁵X¹⁶- wherein:
each of X², X³, X⁴, X⁵, X⁶, X⁷, X⁸, X⁹, X¹⁰, X¹¹, X¹², X¹³, X¹⁴, X¹⁵, and X¹⁶ is an independently selected amino acid of formula NH(R^{a1})-L^{a1}-C(R^{a2})(R^{a3})-L^{a2}-COOH as described in the present disclosure;
X¹ is Xaa^{N}, such as Asp or Glu;
X³ is Asp or Glu;
X⁵ is Xaa^{H}, such as Ala;
X⁶ is Tyr;
X⁷ is His;
X⁸ is Xaa^{H}, such as Ala;
X⁹ is Gly;
X¹⁰ is Asp or Glu;
X¹¹ is Xaa^{H}, such as Leu;
X¹² is Xaa^{H}, such as Val;
X¹³ is Tyr;
X¹⁵ is an amino acid residue comprising a polar uncharged side chain (e.g., at physiological pH, “polar uncharged amino acid residue”, Xaa^{L}), such as Val;
two non-neighboring amino acid residues are connected by L^{b} (e.g., L^{b} connects two alpha-carbon atoms of two different amino acid residues);
X² are connected to X¹⁶ by L^{b} (e.g., both X² and X¹⁶ are Cys, and the two —SH groups of their side chains form —S—S—, such as L^{b} is —CH₂—S—S—CH₂—);
X⁴ are connected to X¹⁴ by L^{b} (e.g., both X⁴ and X¹⁴ are Cys, and the two —SH groups of their side chains form —S—S—, such as L^{b} is —CH₂—S—S—CH₂—); or
a combination thereof.

17. The compound of any one of claims 1-10, wherein one or more ABM independently is or comprises Xaa^{A}XaaXaa^{P}Xaa^{p}, Xaa^{P}Xaa^{P}XaaXaa^{A}, Xaa^{P}Xaa^{A}Xaa^{P}, XaaXaa^{A}Xaa^{P}, Xaa^{P}Xaa^{A}Xaa^{P}XaaXaa^{P}Xaa^{A}Xaa^{P}, Xaa^{P}Xaa^{P}Xaa^{A}Xaa^{A}Xaa^{P}, Xaa^{P}Xaa^{P}Xaa^{A}, or Xaa^{A}Xaa^{A}Xaa^{P}, wherein:
Xaa^{A} is an aromatic amino acid residue with a side chain that comprises an aromatic group,
Xaa^{P} is a positively charged amino acid residue comprising a positively charged side chain (e.g., positively charged at a physiological pH of about 7.4), and
Xaa^{N} is a negatively charged amino acid residue comprising a negatively charged side chain (e.g., negatively charged at a physiological pH about 7.4).

18. The compound of any one of claims 1-18, wherein the amino acid has the structure:
NH(R^{a1})-L^{a1}-C(R^{a2})(R^{a3})-L^{a2}-COOH or N(R^{a1})-L^{a1}-C(R^{a2})(R^{a3})-L^{a2}-CO-, or a pharmaceutically acceptable salt thereof, wherein:
each R^{a1}, R², and R^{a3} is independently -L^{a}-R′;
each L^{a1}and L^{a2} is independently L^{a};
each L^{a} is independently a covalent bond, or an optionally substituted bivalent group selected from C₁-C₂₀ aliphatic and C₁-C₂₀ heteroaliphatic having 1-5 heteroatoms, wherein one or more methylene units of the bivalent group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₁₋₃₀ aryl, C₁₋₃₀ arylaliphatic, C₁₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms,
wherein each heteroatom of the amino acid is independently selected from oxygen, nitrogen, sulfur, phosphorus, and silicon.

19. The compounds of any one of claims 9-19, wherein two or more side chains of the amino acid residues (e.g., R^{a2} or R^{a3} of one amino acid residue with R^{a2} or R^{a3} of another amino acid residue) are take together to form a bridge (e.g., two cysteine residues in a ABM or a TBT form a —S—S— bridge as typically observed in natural proteins),

20. The compound of claim 20, wherein the bridge has the structure L^{b}, wherein:
each L^{b} is independently an optionally substituted bivalent group selected from C₁-C₂₀ aliphatic and C₁-C₂₀ heteroaliphatic having 1-5 heteroatoms, wherein one or more methylene units of the group are optionally and independently replaced with —C(R′)₂-, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₁₋₃₀ aryl, C₁₋₃₀ arylaliphatic, C₁₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon; and
each heteroatom of L^{b} is independently selected from oxygen, nitrogen, sulfur, phosphorus, and silicon.

21. The compound of any one of claims 1-10, or a pharmaceutically acceptable salt thereof, wherein:
(a) one or more ABM has the chemical structure: 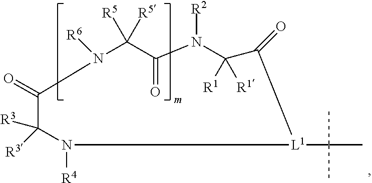
wherein:
each R¹, R³ and R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms, a 5-6 membered monocyclic heteroaromatic ring having 1-4 heteroatoms, or an 8-10 membered bicyclic heteroaromatic ring having 1-5 heteroatoms; or:
R¹ and R^{1″} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms;
R³ and R^{3′} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms;
R⁵ and R^{5″} are optionally taken together with their intervening carbon atom to form a 3-8 membered optionally substituted saturated or partially unsaturated spirocyclic carbocyclic ring, or a 3-8 membered saturated or partially unsaturated spirocyclic heterocyclic ring having 1-2 heteroatoms; or
two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)-, —N(R)C(O)-, —S(O)—, —S(O)₂—, or -Cy¹-, wherein each -Cy¹-is independently a 5-6 membered heteroarylenyl with 1-4 heteroatoms;
each R^{1′}, R^{3′} and R^{5′} is independently hydrogen or optionally substituted C₁₋₃ aliphatic;
each R², R⁴ and R⁶ is independently hydrogen, or optionally substituted C₁₋₄ aliphatic, or:
R² and R¹ are optionally taken together with their intervening atoms to form a 4-11 membered, optionally substituted saturated or partially unsaturated monocyclic heterocyclic ring having 1-2 heteroatoms;
R⁴ and R³ are optionally taken together with their intervening atoms to form a 4-11 membered optionally substituted saturated or partially unsaturated monocyclic heterocyclic ring having 1-2 heteroatoms; or
an R⁶ group and its adjacent R group are optionally taken together with their intervening atoms to form a 4-11 membered optionally substituted saturated or partially unsaturated monocyclic heterocyclic ring having 1-2 heteroatoms;
L¹ is a trivalent linker moiety that connects L and 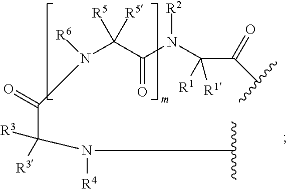
each m and n is independently 1, 2, 3, 4, 5, 6, 7, 8, 9, or 10; and
each heteroatom of the ABM is independently selected from nitrogen, oxygen, and sulfur;
(b) one or more ABM has the chemical structure: 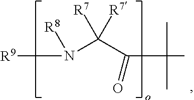
wherein:
each R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms, a 5-6 membered monocyclic heteroaromatic ring having 1-4 heteroatoms, or an 8-10 membered bicyclic heteroaromatic ring having 1-5 heteroatoms; or:
R⁷ and R^{7′} are optionally taken together with their intervening carbon atom to form a 3-8 membered optionally substituted saturated or partially unsaturated spirocyclic carbocyclic ring, or a 3-8 membered optionally substituted saturated or partially unsaturated spirocyclic heterocyclic ring having 1-2 heteroatoms;
each R^{7′} is independently hydrogen or optionally substituted C₁₋₃ aliphatic;
each R⁸ is independently hydrogen, or optionally substituted C₁₋₄ aliphatic, or:
R⁸ and its adjacent R⁷ group are optionally taken together with their intervening atoms to form a 4-11 membered optionally substituted saturated or partially unsaturated monocyclic heterocyclic ring having 1-2 heteroatoms;
each R⁹ is independently hydrogen, optionally substituted C₁₋₃ aliphatic, or —C(O)-(optionally substituted C₁₋₃ aliphatic);
o is 1, 2, 3, 4, 5, 6, 7, 8, 9, or 10; and
each heteroatom of the ABM is independently selected from nitrogen, oxygen, and sulfur;
(c) a combination thereof.

22. The compound of claim 21, or a pharmaceutically acceptable salt thereof, wherein:
(a) each L¹ is independently selected from: 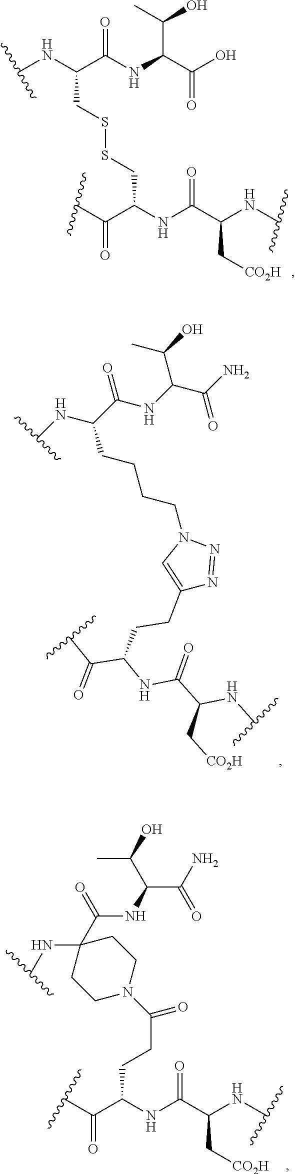 [SMILES: *N[C@@H](CC(=O)O)C(=O)N[C@@H](CSSC[C@H](N*)C(=O)N[C@H](C(=O)O)[C@@H](C)O)C(*)=O ; *N[C@@H](CCCCn1cc(CC[C@H](NC(=O)[C@H](CC(=O)O)N*)C(*)=O)nn1)C(=O)NC(C(N)=O)[C@@H](C)O ; *N[C@@H](CC(=O)O)C(=O)N[C@@H](CCC(=O)N1CCC(N*)(C(=O)N[C@H](C(N)=O)[C@@H](C)O)CC1)C(*)=O] 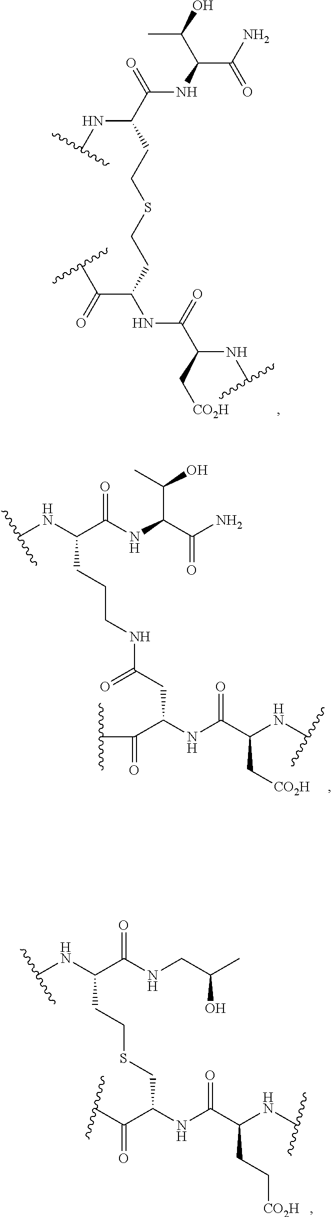 [SMILES: *N[C@@H](CC(=O)O)C(=O)N[C@@H](CCSCC[C@H](N*)C(=O)N[C@H](C(N)=O)[C@@H](C)O)C(*)=O ; *N[C@@H](CC(=O)O)C(=O)N[C@@H](CC(=O)NCCC[C@H](N*)C(=O)N[C@H](C(N)=O)[C@@H](C)O)C(*)=O ; *N[C@@H](CCSC[C@H](NC(=O)[C@H](CCC(=O)O)N*)C(*)=O)C(=O)NC[C@@H](C)O] 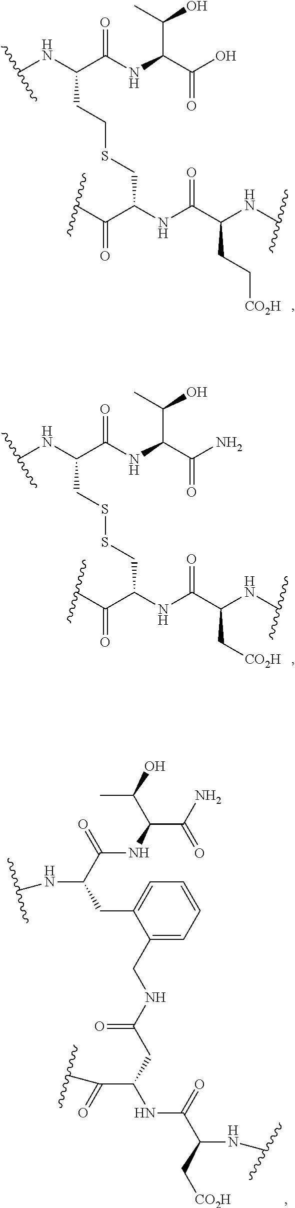 [SMILES: *N[C@@H](CCC(=O)O)C(=O)N[C@@H](CSCC[C@H](N*)C(=O)N[C@H](C(=O)O)[C@@H](C)O)C(*)=O ; *N[C@@H](CC(=O)O)C(=O)N[C@@H](CSSC[C@H](N*)C(=O)N[C@H](C(N)=O)[C@@H](C)O)C(*)=O ; *N[C@@H](CC(=O)O)C(=O)N[C@@H](CC(=O)NCc1ccccc1C[C@H](N*)C(=O)N[C@H](C(N)=O)[C@@H](C)O)C(*)=O] 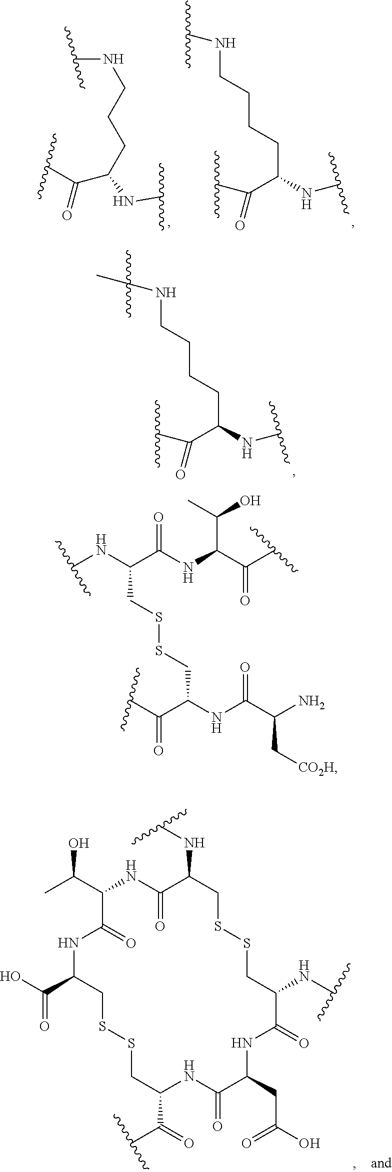 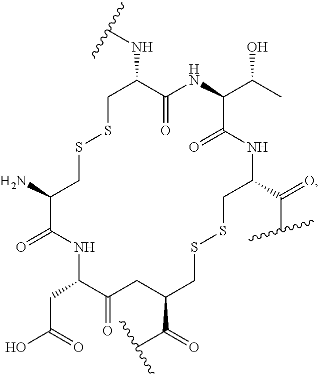 [SMILES: *N[C@H]1CSSC[C@H](N)C(=O)N[C@@H](CC(=O)O)C(=O)C[C@H](C(*)=O)CSSC[C@@H](C(*)=O)NC(=O)[C@H]([C@@H](C)O)NC1=O]
wherein the N-terminus is optionally acetylated (—COCH₃) and the C-terminus is optionally amidated (—NH₂);
(b) each L¹ is independently selected from: 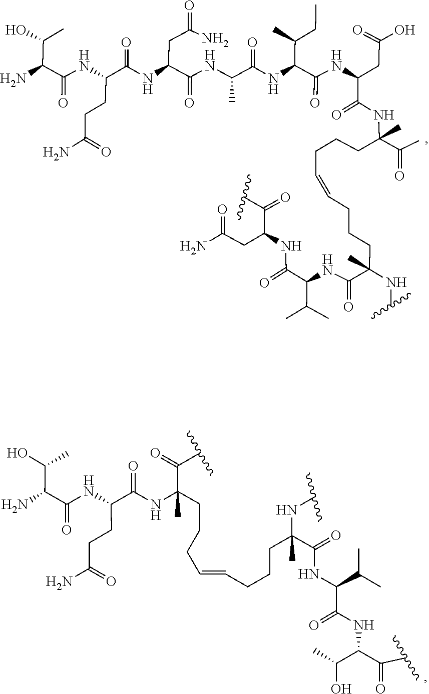 [SMILES: *N[C@@](C)(CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(C)=O)C(=O)N[C@H](C(=O)N[C@@H](CC(N)=O)C(*)=O)C(C)C ; *N[C@@](C)(CCC/C=C/CCC[C@](C)(NC(=O)[C@H](CCC(N)=O)NC(=O)[C@H](N)[C@@H](C)O)C(*)=O)C(=O)N[C@H](C(=O)N[C@H](C(*)=O)[C@@H](C)O)C(C)C] 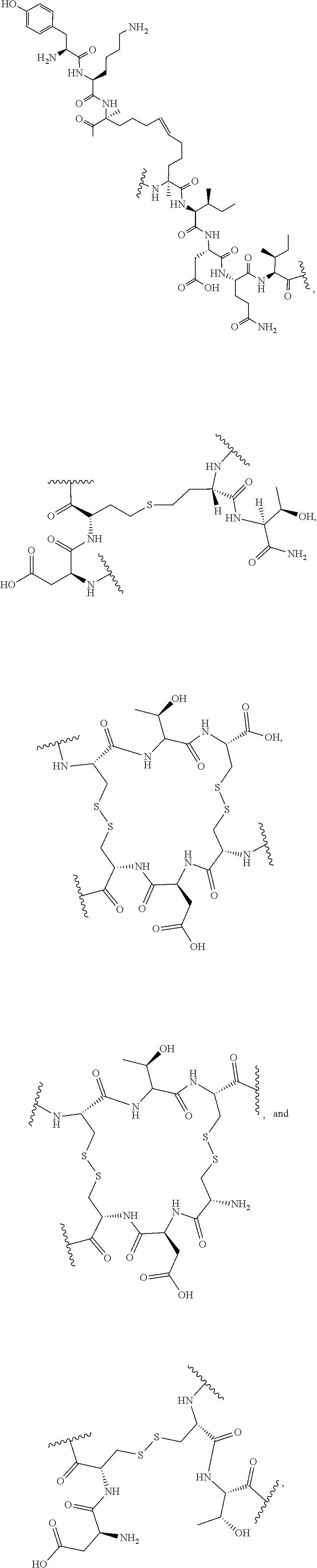
wherein the N-terminus is optionally acetylated (—COCH₃) and the C-terminus is optionally amidated (—NH₂); or
(c) a combination thereof.

23. The compound of any one of claims 1-23, or a pharmaceutically acceptable salt thereof, wherein
(a) one or more ABM, independently, is or comprises an amino acid sequence selected from APAR, PARA, HWRGWA, WGRR, RRGW, NRFRGKYK, NARKFYK, RHRFNKD, TY, TYK, RTY, RTYK, PAM, WHL, ELVW, AWHLGELVW, DCAWHLGELVWCT, D-Cyclo[CAWHLGELVWC]T, DpLpAWHLGELVW, DpLpDCAWHLGELVWCT, CDCAWHLGELVWCTC, Ac-ASYHLGELVW[1,2,3,4-tetrahydroisoquinoline-3-carboxylic acid][Aib]C, Cyclo[Ac-ASYHLGELVW[1,2,3,4-tetrahydroisoquinoline-3-carboxylic acid][Aib]C], Ac-ASYHLGELVW[1,2,3,4-tetrahydroisoquinoline-3-carboxylic acid][Aib]C[dE], Cyclo[Ac-ASYHLGELVW[1,2,3,4-tetrahydroisoquinoline-3-carboxylic acid][Aib]C][dE], Ac-ASYHLGELVW[1,2,3,4-tetrahydroisoquinoline-3-carboxylic acid][Aib]CE, and Cyclo[Ac-ASYHLGELVW[1,2,3,4-tetrahydroisoquinoline-3-carboxylic acid][Aib]C]E;
(b) one or more ABM is an antibody binding moiety independently selected from Fc-III, Fc-III-4c, FcRM, FcBP-1, FcBP-2, FcRM, and PAM; or
(c) a combination thereof.

24. The compound of any one of claims 1-23, or a pharmaceutically acceptable salt thereof, wherein one or more ABM, independently, is or comprises an ABM from an exemplary compound of the present disclosure, such as an optionally substituted chemical structure independently selected from:

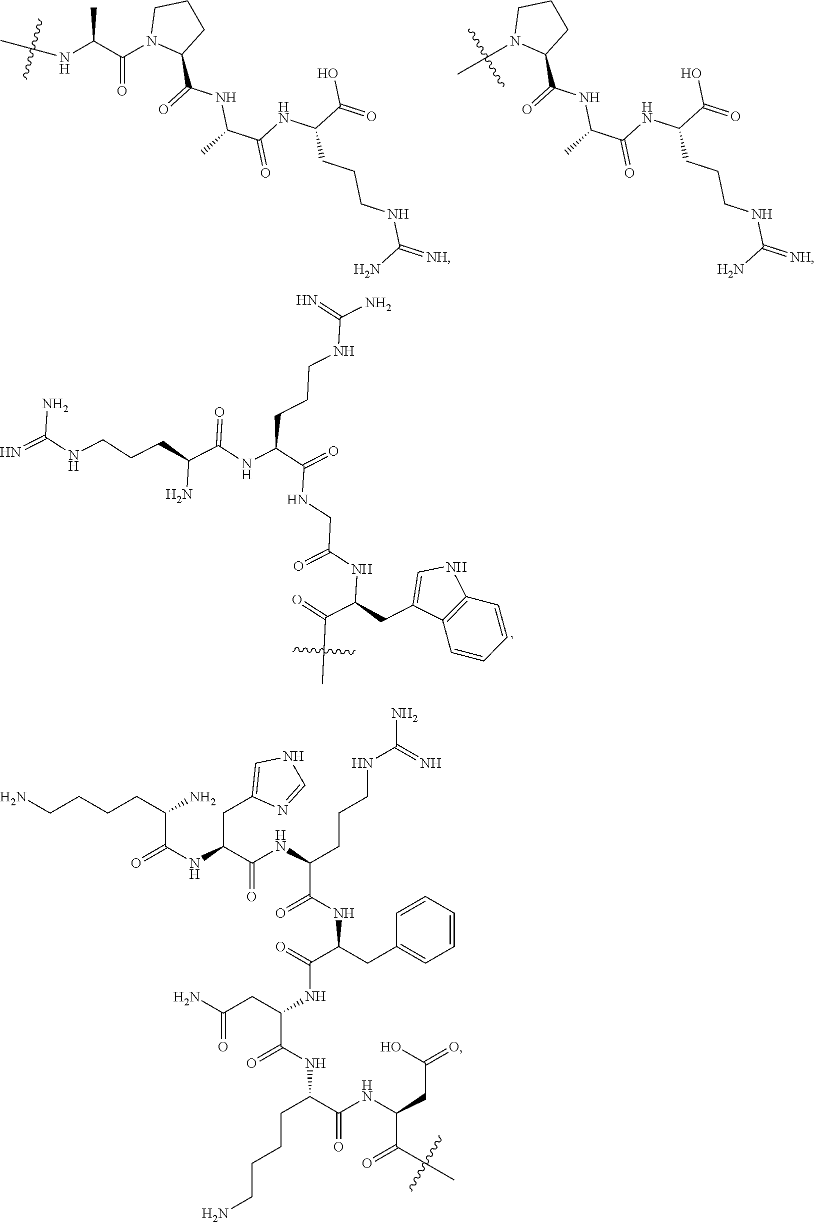

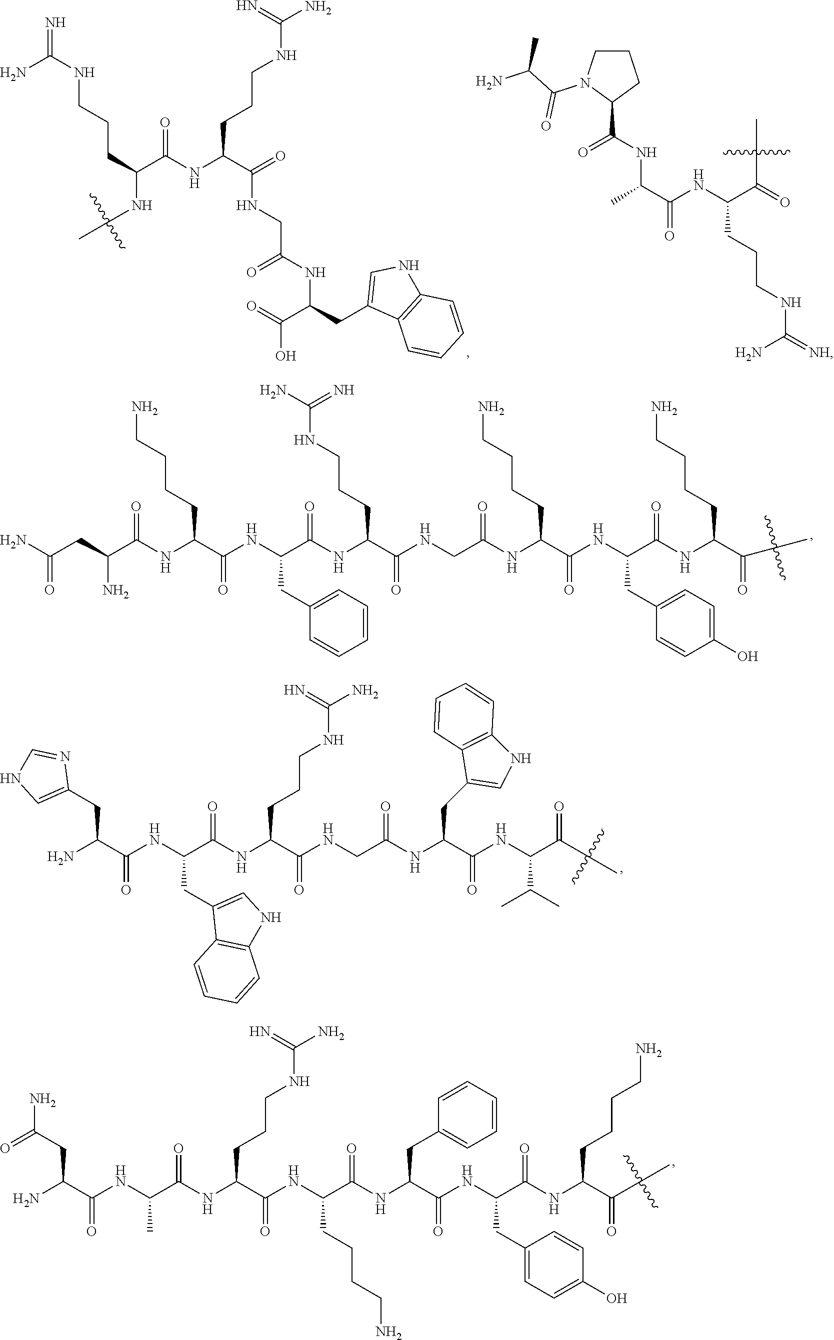

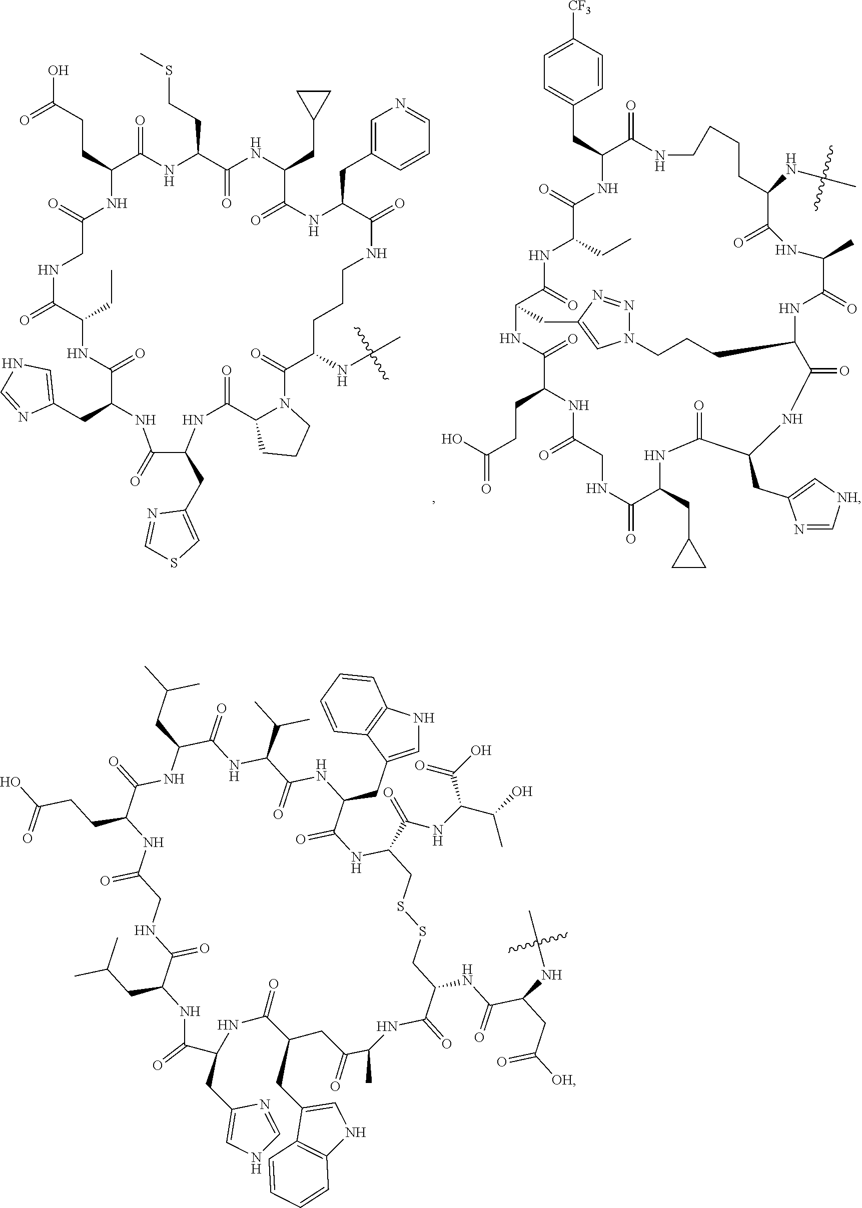

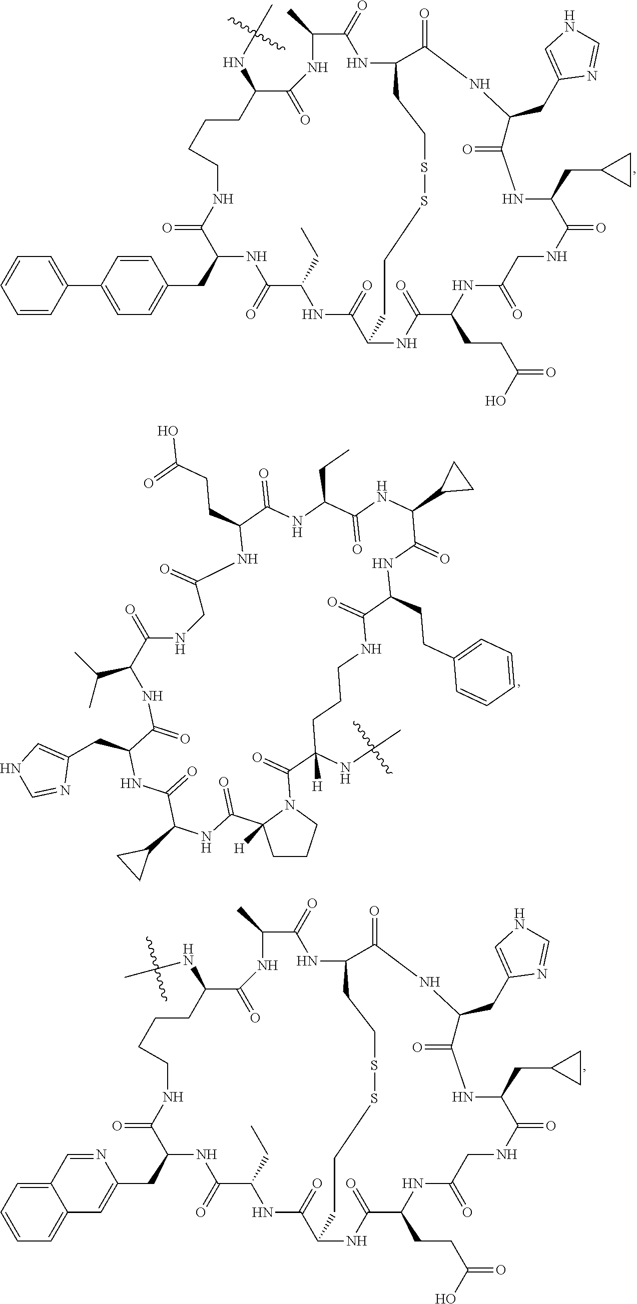

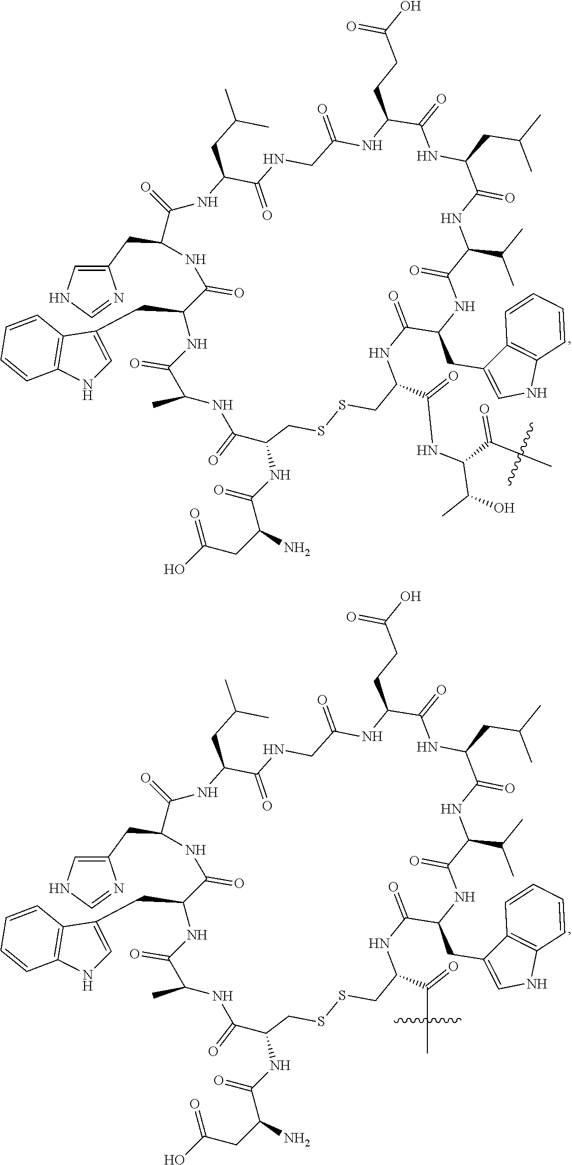

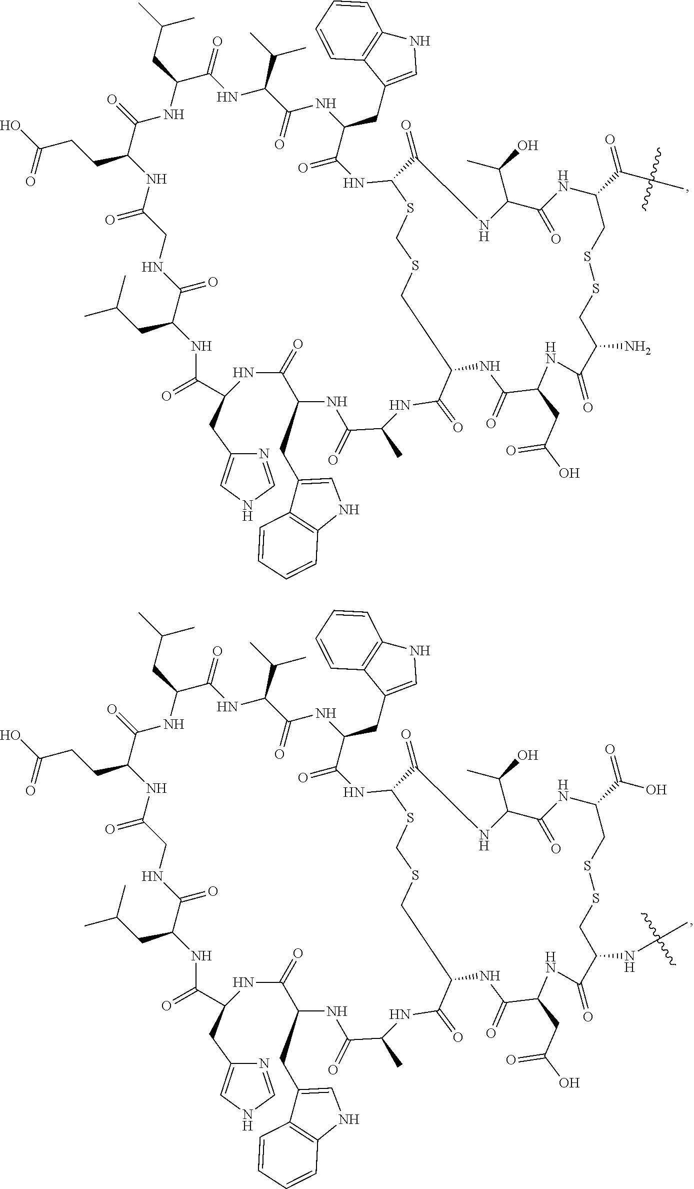

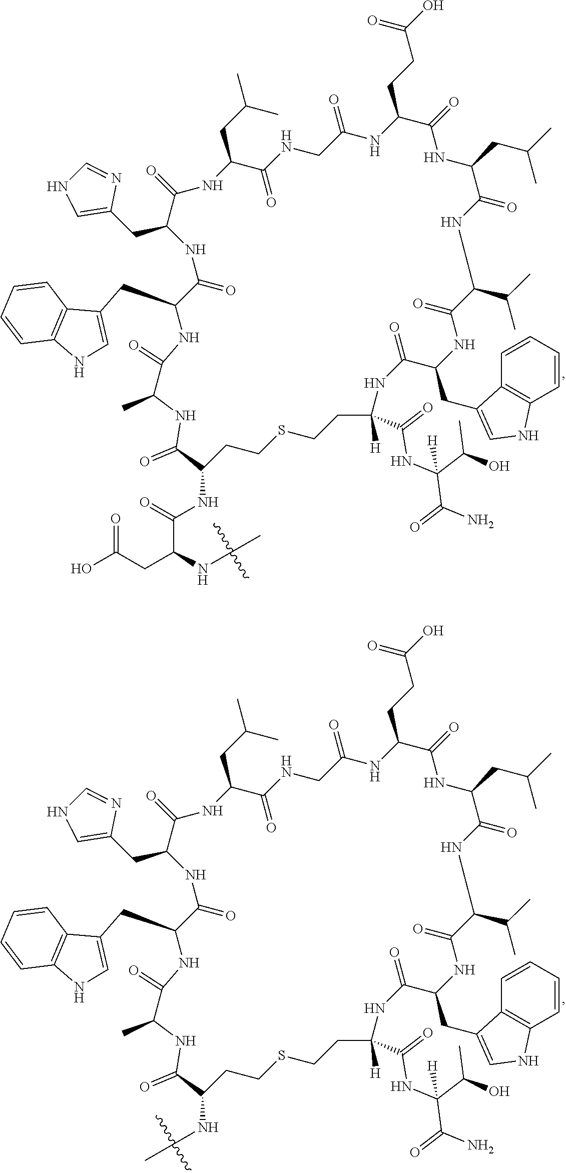

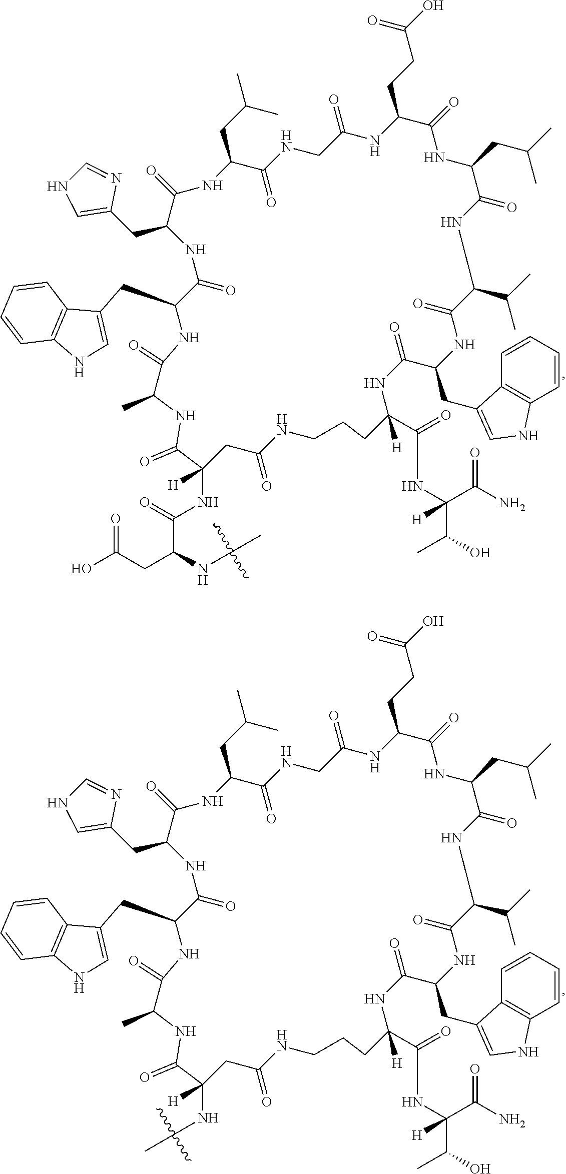

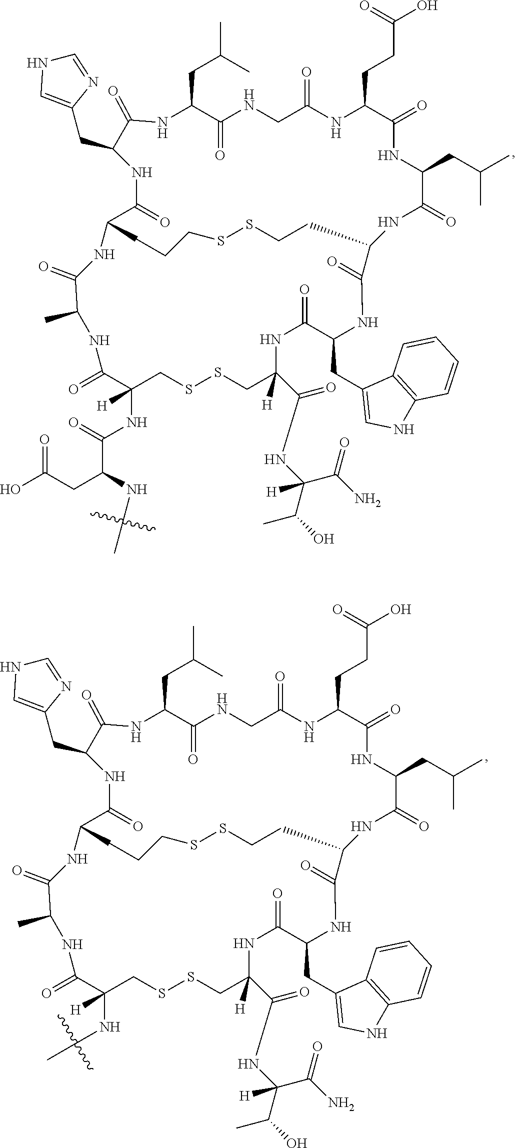 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2 ; *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2]

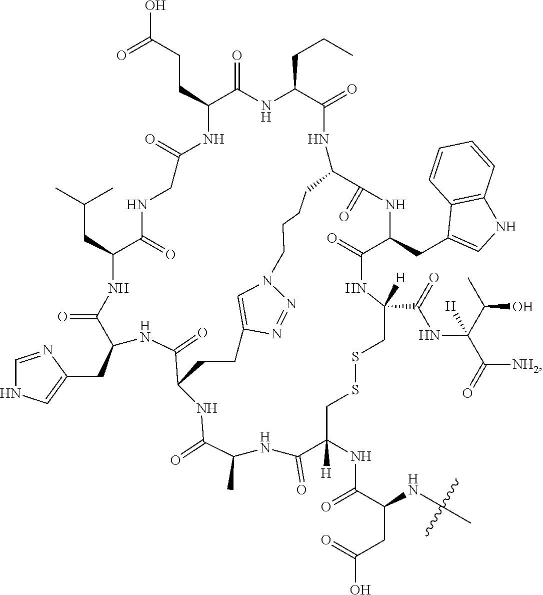 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2]

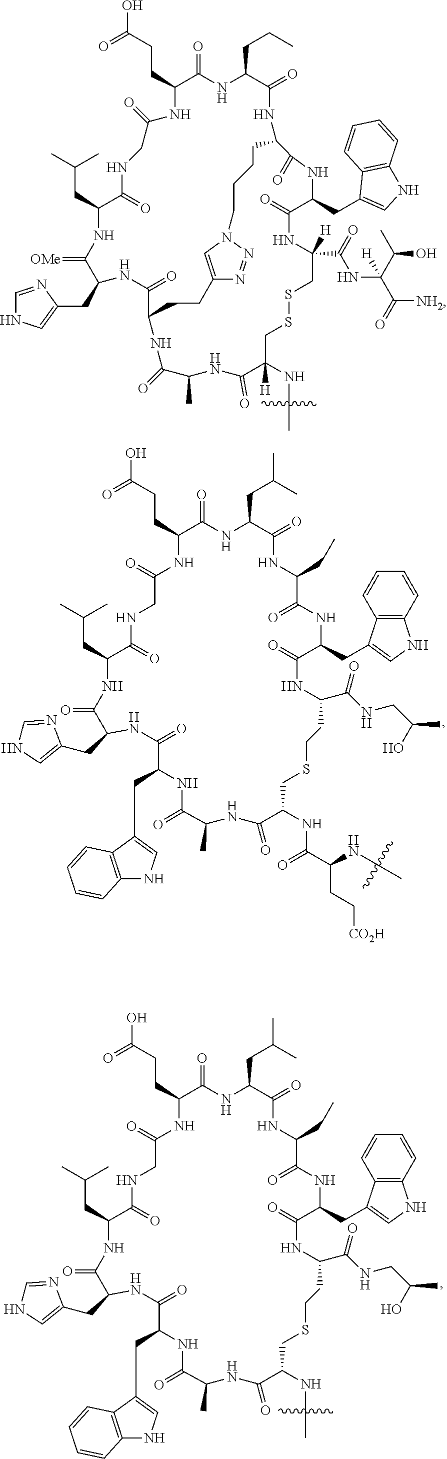

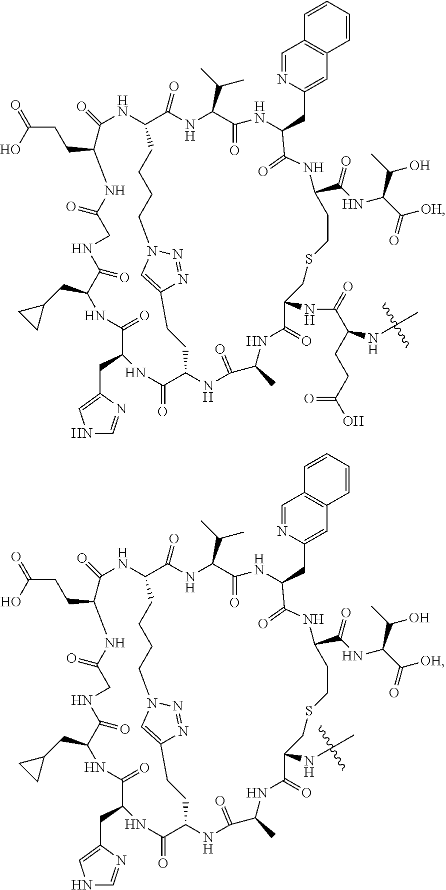 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2 ; *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2]

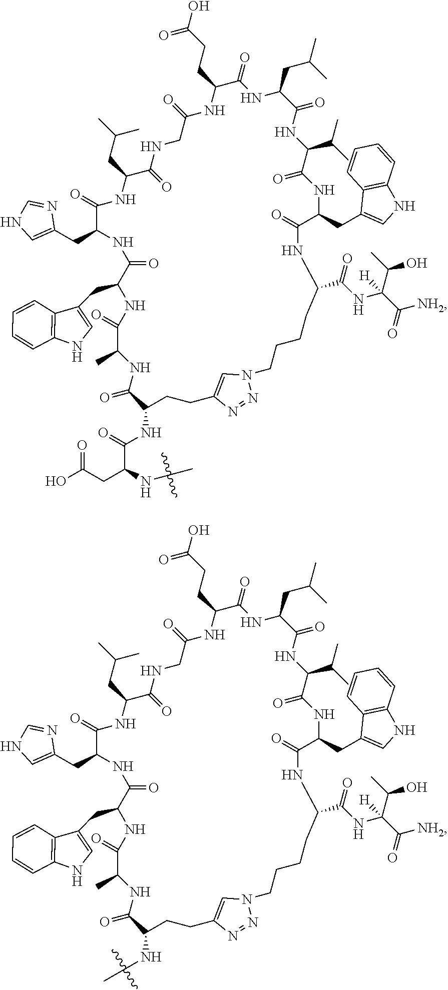

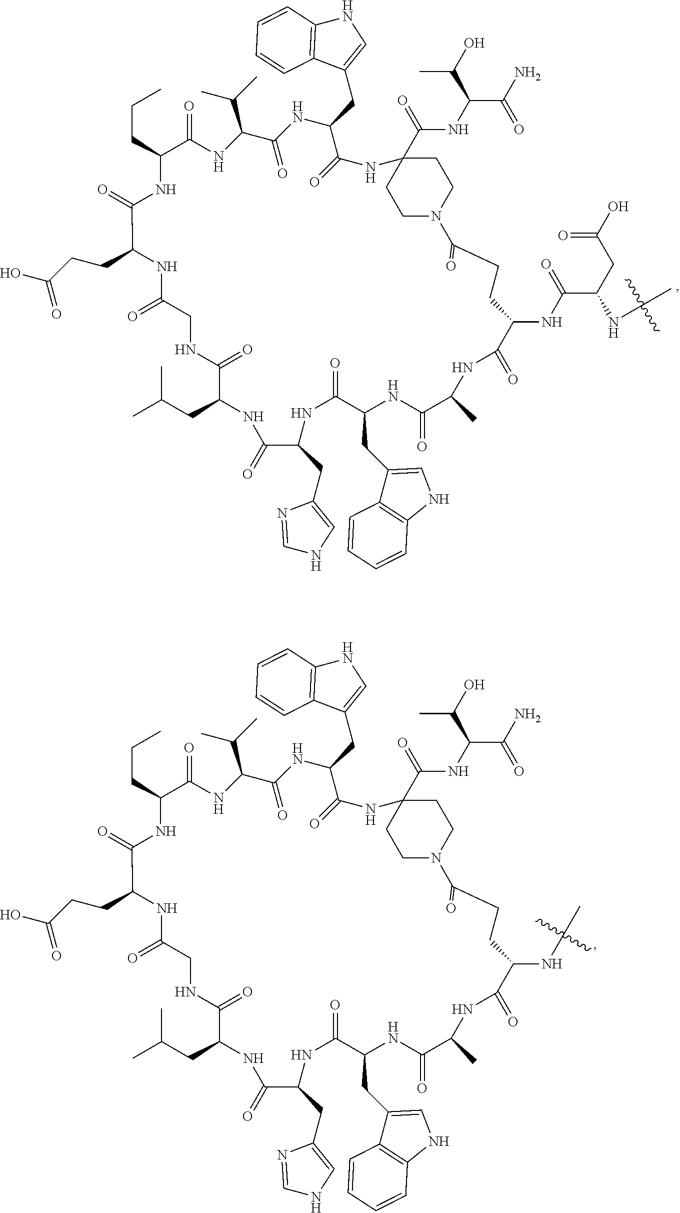 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

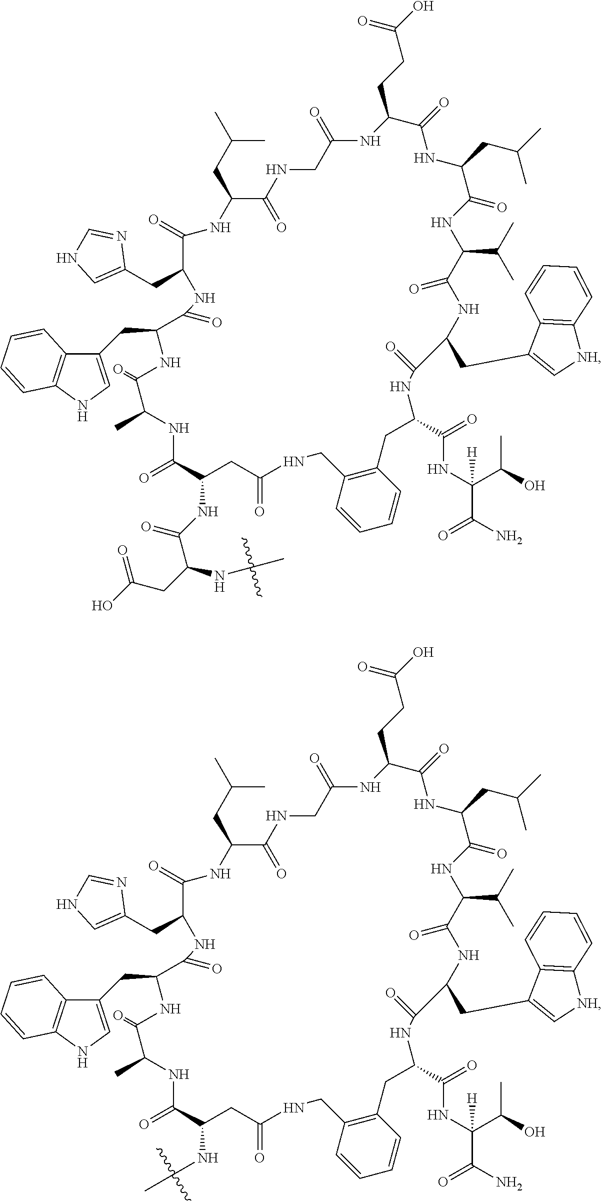

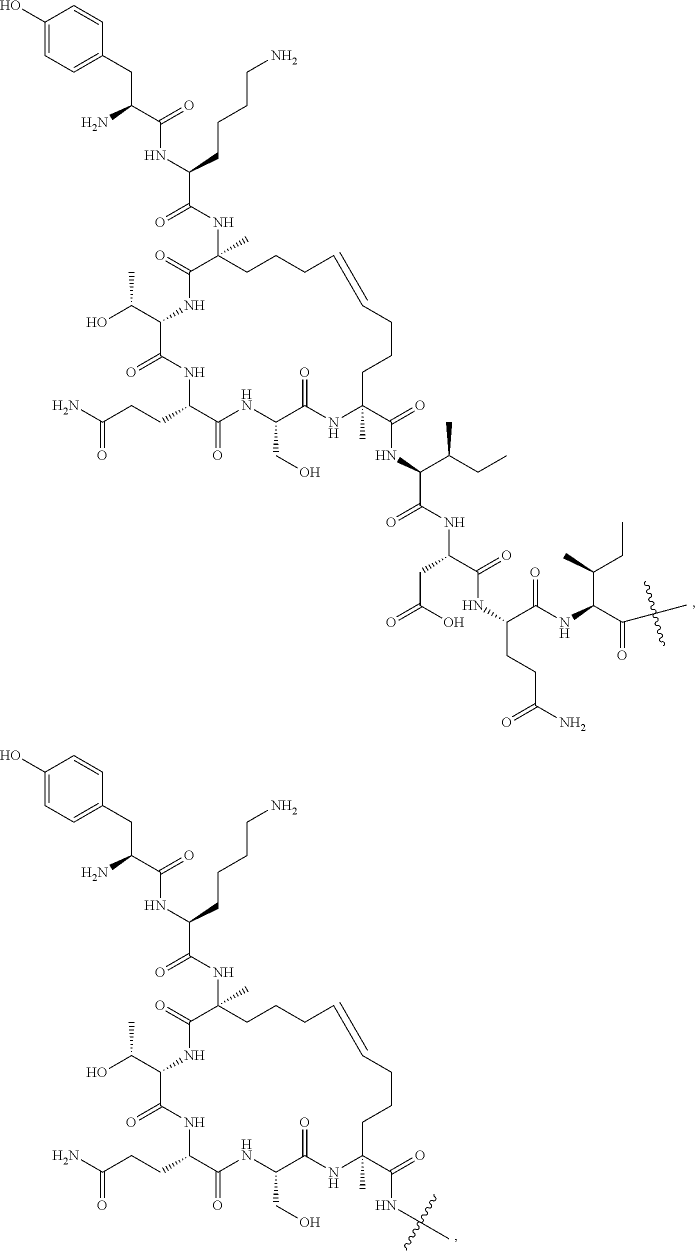 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC ; *NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1]

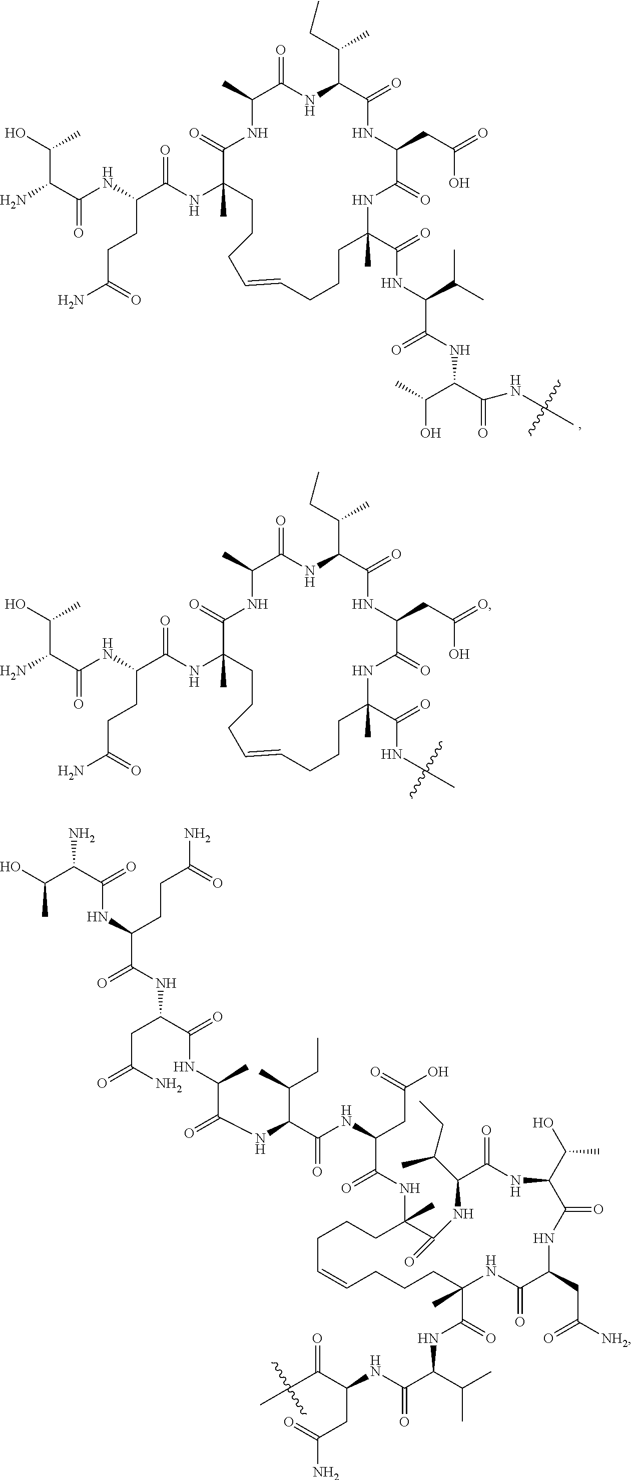

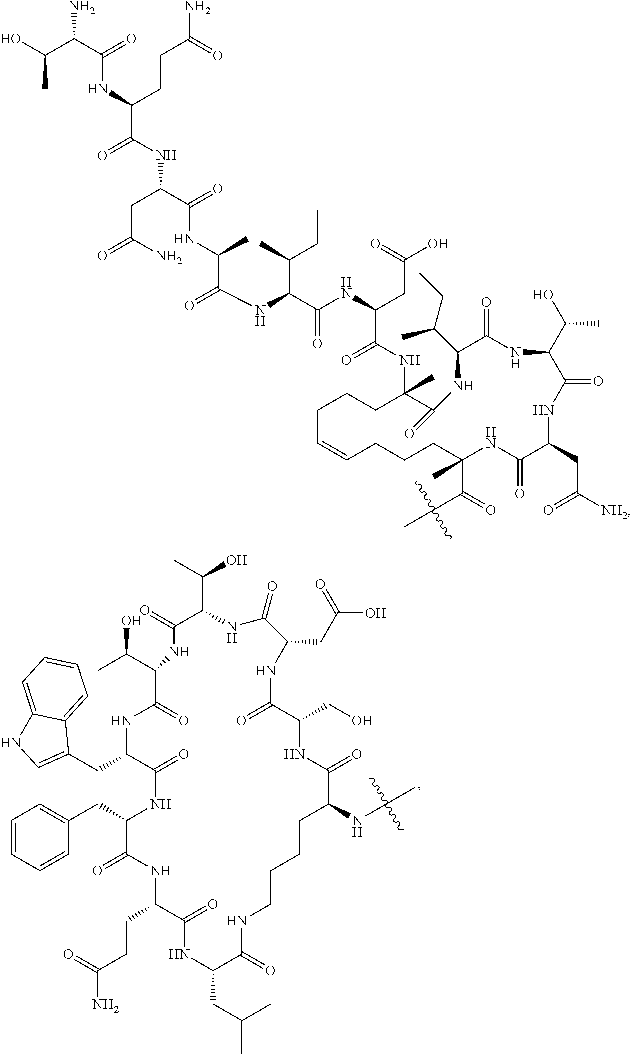 [SMILES: *C(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1 ; *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O]

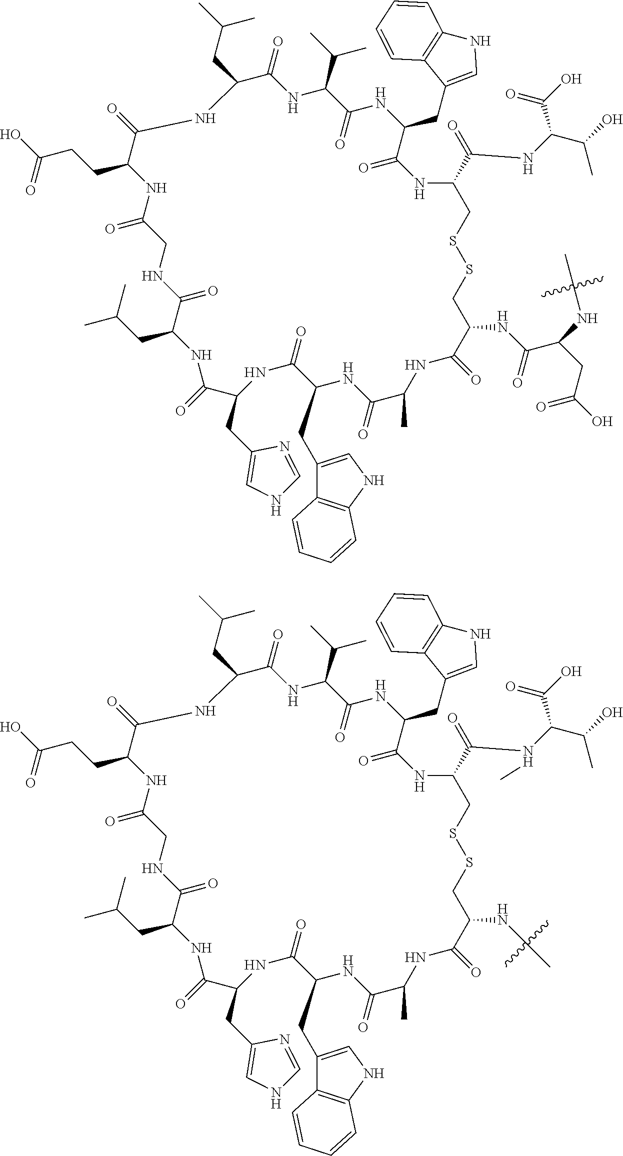 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *N[C@H]1CSSC[C@@H](C(=O)N(C)[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

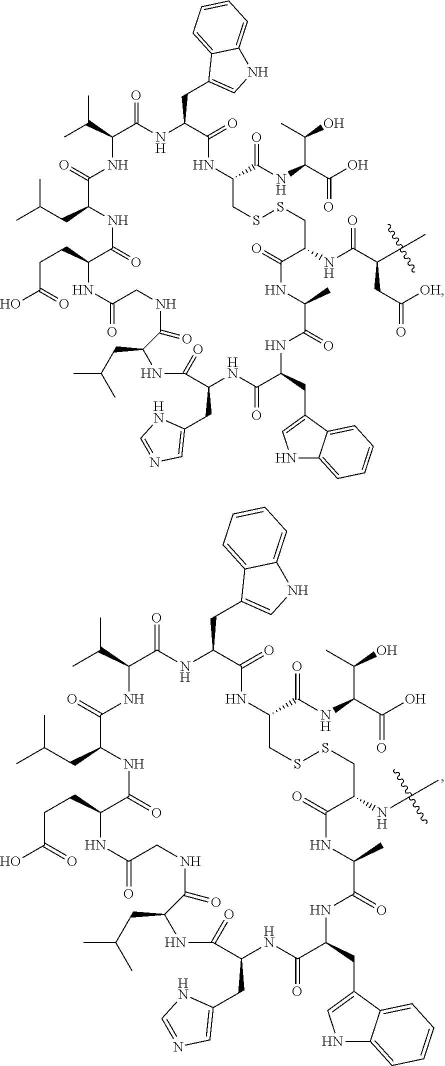 [SMILES: *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O ; *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O]

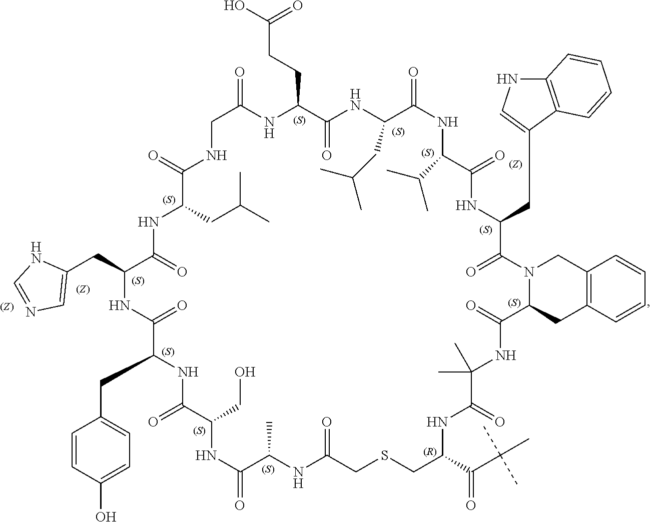

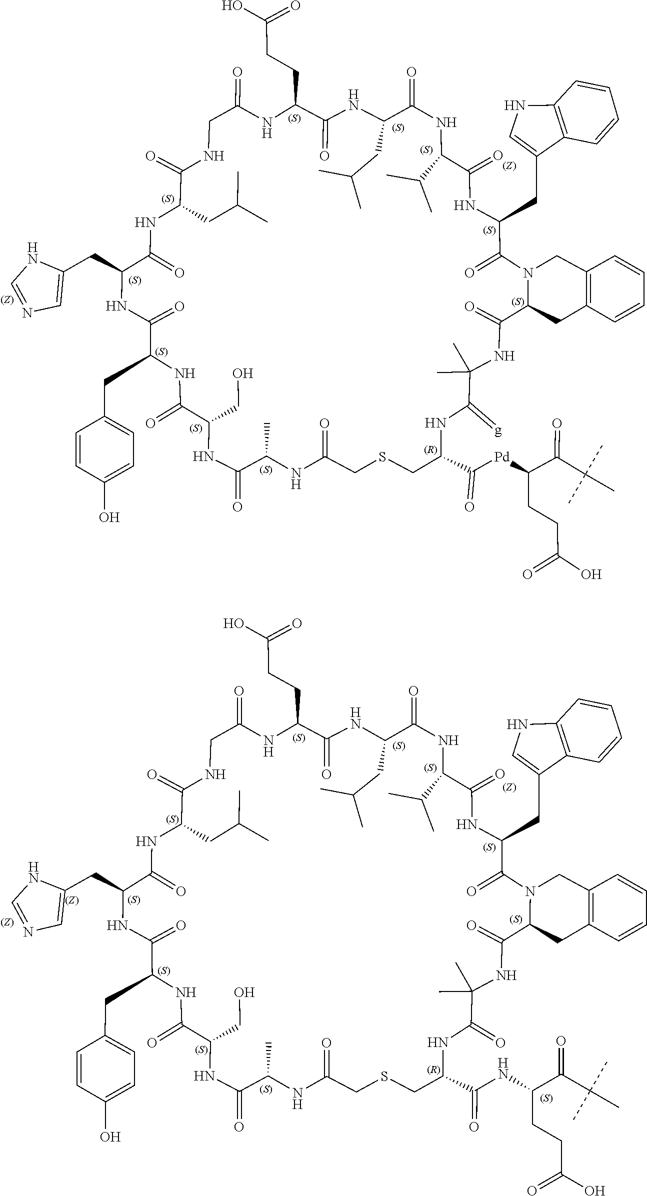

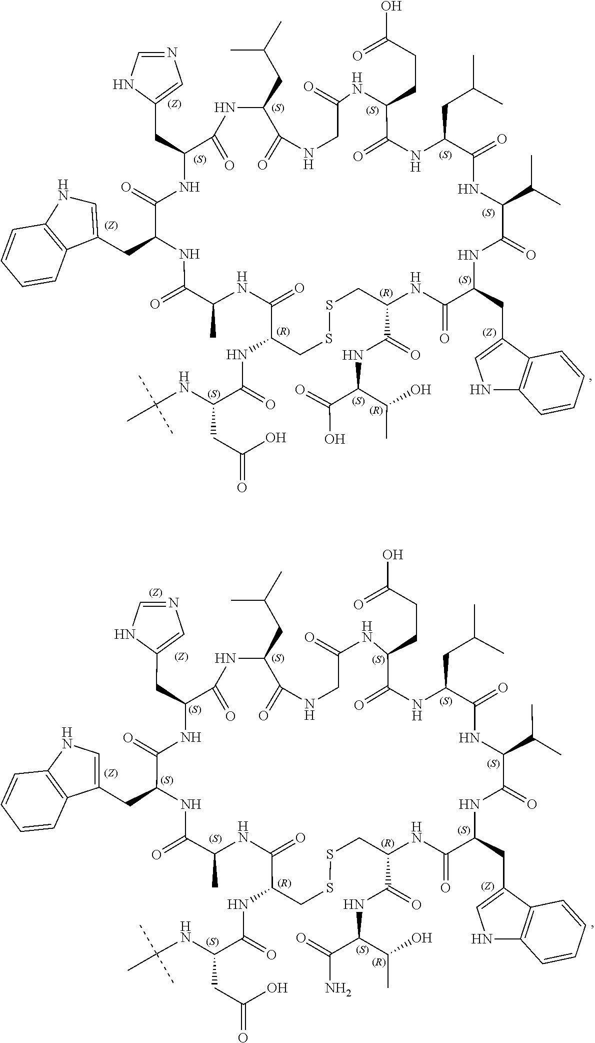

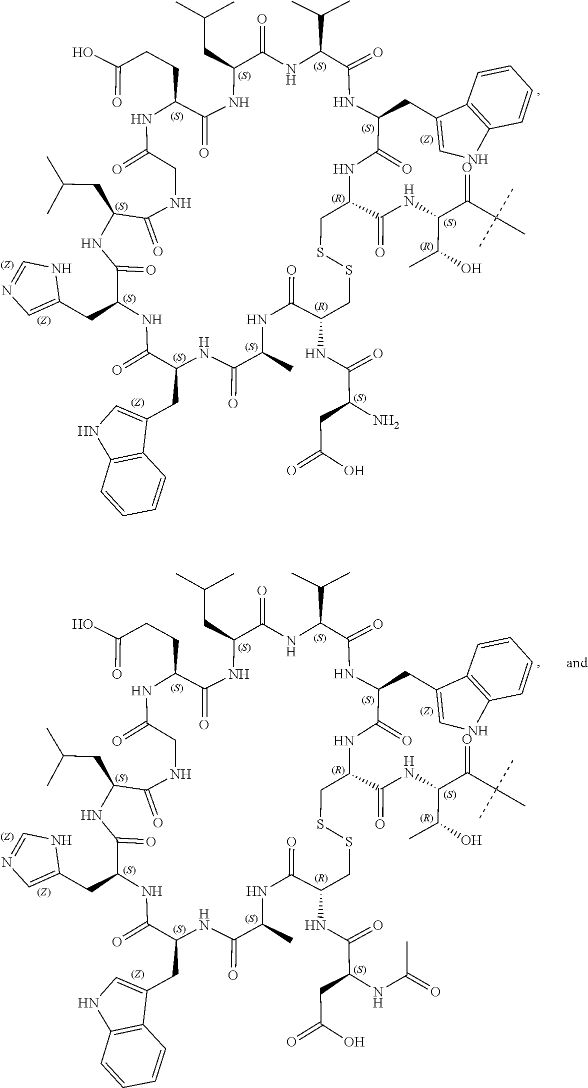

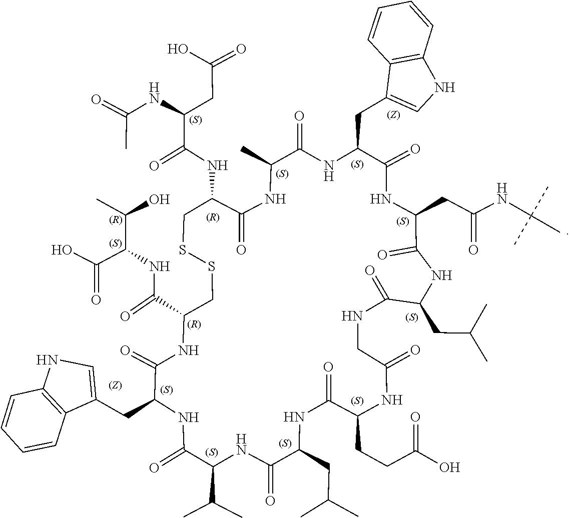

25. The compound of any one of claims 1-25, or a pharmaceutically acceptable salt thereof, wherein the L is an optionally substituted C₁-C₇₀ alkylene group, an optionally substituted C₁-C₆₀ alkylene group, or an optionally substituted C₁-C₅₀ alkylene group (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, C₆₀, C₆₁, C₆₂, C₆₃, C₆₄, C₆₅, C₆₆, C₆₇, C₆₈, C₆₉, or C₇₀ alkylene), wherein:
the L optionally comprises one to fourteen -C₁₋₂₅ alkynelene-ABM covalently linked to the C₁₋₇₀ alkynelene group; and
the L optionally comprises one to fourteen -C₁₋₂₅ alkynelene-TBM covalently linked to the C₁₋₇₀ alkynelene group;
each CH₂ group is optionally replaced with a group independently selected from CR^{L1}R^{L2}, O, S, SO, SO₂, NH, CO, CH═CH, C≡C, 5-7 membered heterocyle, and 3-8 membered carbocycle;
each R^{L1} is independently selected from H, an amino acid side chain, or an optionally substituted linear or branched C₁₋₅ alkyl with one or two CH₂ groups are optionally replaced with a group independently selected from O, S, SO, SO₂, or NH, wherein the C₁₋₅ alkyl group is optionally substituted with a hydroxyl, —NH₂, —NH₃, oxo (═O), an optionally substituted 5-7 membered carbocycle or aryl, or an optionally substituted 3-10 membered heterocycle or heteroaryl;
each R^{L2} is independently selected from H and optionally substituted linear or branched C₁₋₄ alkyl (e.g., methyl);
one or two CH₂ groups of the C₁₋₅₀ alkylene, the C₁₋₂₅ alkylene-ABM, the C₁₋₂₅ alkylene-TBM, or a combination thereof is optionally replaced with a [CON], wherein the [CON] is a bond or an optional chemical moiety that forms from a reaction production;
the L is optionally substituted with one or more (e.g., 1, 2, 3, 4, 5, 6, 7, 8, 9, 10 or more) groups independently selected from H, halogen, hydroxyl, cyano, oxo (═O), amino, imino (═NH), —COOH, C₁₋₄ alkyl, C₁₋₄haloalkyl, and C₁₋₄alkoxy.

26. The compound of claim 26, or a pharmaceutically acceptable salt thereof, wherein each [CON], independently, is or comprises: 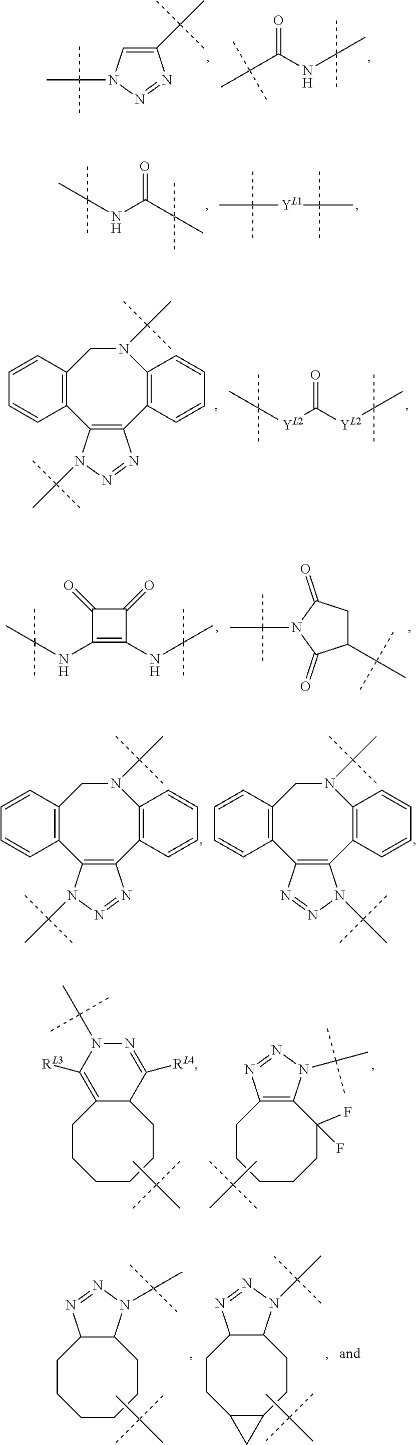 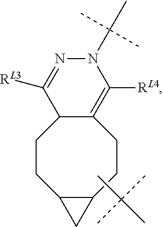
wherein:
each Y¹ is independently CH₂, O, S, NR^{L5} (e.g. NC₁-C₃alkyl)), C(O), S(O), S(O)₂, —S(O)₂O, —OS(O)₂, or OS(O)₂O;
each Y² is independently O, S, or NR^{L5};
each R^{L5} is independently H, C₁-C₃ alkyl, C₁-C₃ alkanoyl, or —C(O)(C₁-C₃alkyl) group;
each R^{L6} is independently H or C₁-C₃ alkyl group (preferably H); and
R^{L3} and R^{L4} are each independently H, methyl, a bond (e.g., for attachment to ABM, L, or TBM);
or a dioxo or diamide group according to the structure: 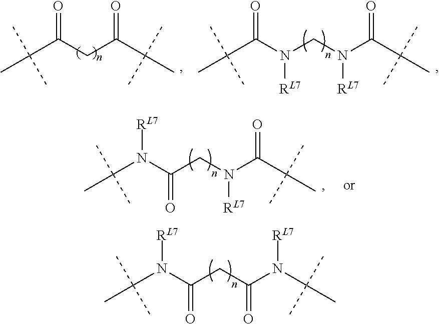
(e.g., one end is for attachment to ABM, L, or TBM), wherein each R^{L7} is independently H or a C₁₋₃ alkyl group (preferably H); and n of [CON] is an integer from 1 to 10 (n is preferably 1, 2, 3, 4, 5, or 6).

27. The compound of any one of claims 1-27, or a pharmaceutically acceptable salt thereof, wherein the L comprises amino acids (e.g., one or more amino acids including G, E, A, K, Q, A, P, or a combination thereof, such as -G_{Ln}-, -QAP_{Ln}-, -GQAP_{Ln}-, -PAS_{Ln}-, or a combination thereof, wherein Ln is an integer from 1 to 15 (such as, 1, 2, 3, 4, 5, 6, 7, 8, 9, 10, 11, 12, 13, 14, or 15).

28. The compound of any one of claims 1-27, or a pharmaceutically acceptable salt thereof, wherein the L is or comprises:
-GG-, -GGG-, -GGGG-, -GGGGGG-, -GGGGGGG-, -GGGGGGGG-, EAAAKEAAAK-, -QAP-, —[NH—(CH₂CH₂O)—CH₂CH₂CO]₂—, —CO—(CH₂CH₂O)₈—CH₂CH₂CONH—(CH₂CH₂O)₈—CH₂CH₂CO—, -GQAPGQAP-, —NH—(CH₂CH₂O)₈—CH₂CH₂CO—, —[CO—(CH₂CH₂O)₈—CH₂CH₂CO]—, —NH-PEG₂-NH—, —NH—CH₂CH₂CH₂—CO—, —NH—(CH₂CH₂O)₄—CH₂CH₂—, —[NH—(CH₂CH₂O)₈—CH₂CH₂CO]₂O—, -PASPASPASPAS-, —[NH—(CH₂CH₂O)₈—CH₂CH₂C═O]₂—(CH₂CH₂O)₂—CH₂CH₂COO— or —[NH—(CH₂CH₂O)₈—CH₂CH₂C═O]₂—(CH₂CH₂O)₂—CH₂CH₂CO—,

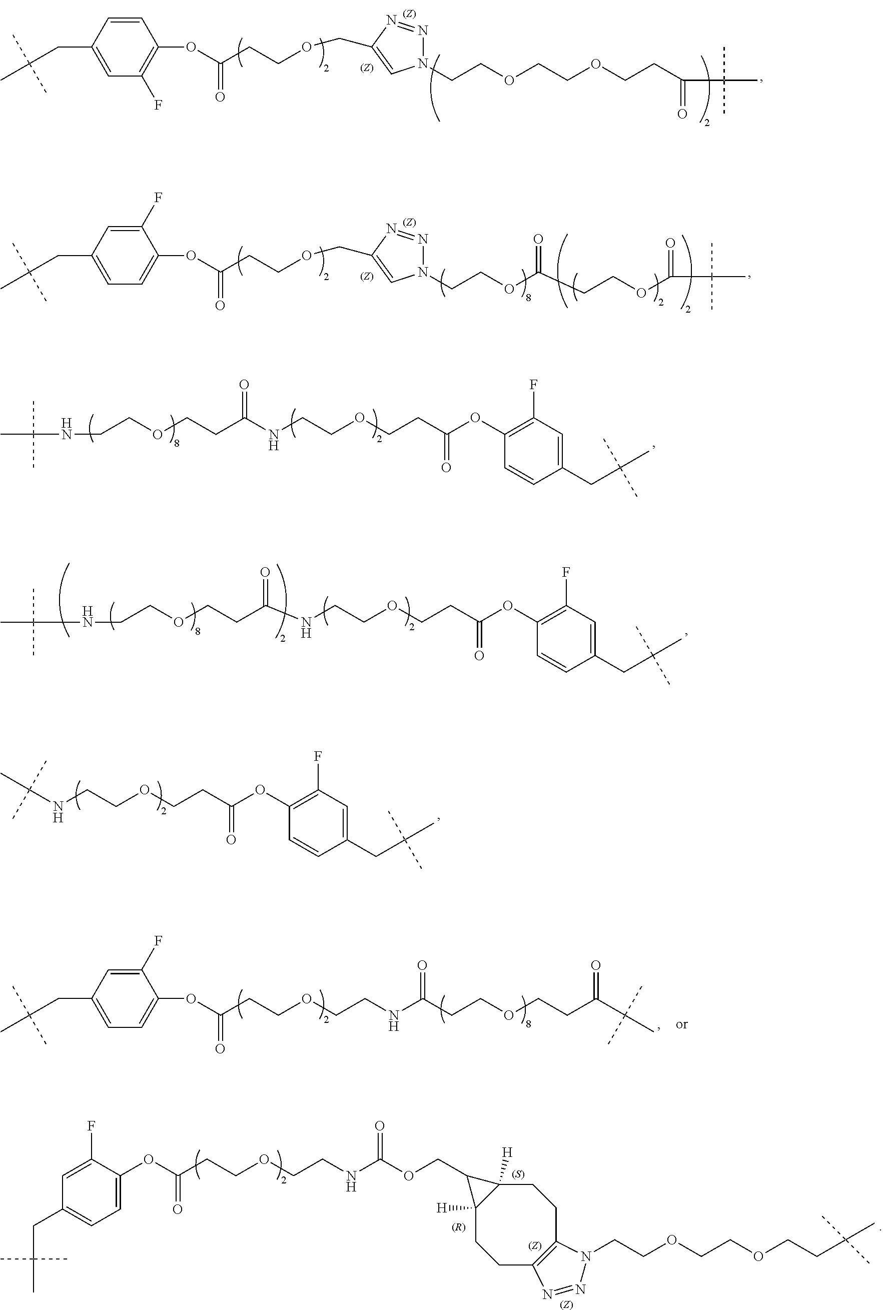

29. The compound of any one of claims 1-25, wherein the L is a bond.

30. A pharmaceutical composition comprising
one or more (e.g., 1, 2, 3, 4, 5, or more) compounds of any one of claims 1 to 30, or a pharmaceutically acceptable salt thereof, and
a pharmaceutically acceptable carrier, adjuvant, additive, excipient, vehicle, or a combination thereof.

31. A combination comprising:
one or more (e.g., 1, 2, 3, 4, 5, or more) compound of any one of claims 1 to 30, or a pharmaceutically acceptable salt thereof; and
a plurality of natural killer cells.

32. A method for inducing cell death, inhibiting cell growth, and/or reducing the number of cells in a system, the method comprising administering to the system one or more (e.g., 1, 2, 3, 4, 5, or more) compound of any one of claims 1 to 30, or a pharmaceutically acceptable salt thereof, wherein the number of cells in the system is reduced compared to the absence of the compound.

33. The method of claim 33, further comprising administering a plurality of natural killer cells, wherein the system is exposed to the plurality of natural killer cells and the compound, and the number of cells in the system is reduced compared to the absence of the natural killer cells and the compound.

34. A method of treating a coronavirus infection, the method comprising administering to a subject in need thereof one or more (e.g., 1, 2, 3, 4, 5, or more) compound of any one of claims 1 to 30, or a pharmaceutically acceptable salt thereof, wherein the number of coronavirus infected cells is reduced as compared to the absence of the compound.

35. The method of claim 35, further comprising administering to the subject in need thereof a plurality of natural killer cells, wherein the subject is exposed to the plurality of natural killer cells and the compound, and the number of coronavirus infected cells in the subject is reduced as compared to the absence of the natural killer cells and the compound.

36. The method of any one of claims 33-36, wherein the system or the subject is a human, a non-human animal, or a non-human animal useful as a disease model.

37. The method of claim 36 or 37, wherein the compound is administered prior to, concurrent with, and/or subsequent to the plurality of natural killer cells.

38. The method of any one of claims 36-38, wherein the plurality of natural killer cells are expanded natural killer cells, enriched natural killer cells, and/or pre-activated natural killer cells.


SEQUENCE LISTING (109 sequences)

SEQ ID NO: 1 - protein, 37 aa, synthetic construct
  SITE 1..37: note = fully protected
DKEWILQKIYEIMRLLDELGHASMRVSDLIYEFMKKG

SEQ ID NO: 2 - protein, 37 aa, synthetic construct; identical to SEQ ID NO: 1
  ACT_SITE 1: note = N-Acetylaspartic acid

SEQ ID NO: 3 - protein, 64 aa, synthetic construct
NDDELHMLMTDLVYEALHFAKDEEIKKRVFQLFELADKAYKNNDRQKLEKVVEELKELLERLLS

SEQ ID NO: 4 - protein, 56 aa, synthetic construct
  ACT_SITE 1: note = N-acetylaspartic acid
DKEWILQKIYEIMRLLDELGHAEASMRVSDLIYEFMKKGDERLLEEAERLLEEVER

SEQ ID NO: 5 - protein, 55 aa, synthetic construct
  DISULFID 44..54: note = Disulfid at Cys 44 and 54
DKEWILQKIYEIMRLLDELGHAEASMRVSDLIYEFMKKGGGGDCAWHLGELVWCT

SEQ ID NO: 6 - protein, 55 aa, synthetic construct; identical to SEQ ID NO: 5

SEQ ID NO: 7 - protein, 56 aa, synthetic construct
  SITE 1: note = universal antibody binding terminus
DCAWILQKIYEIMRLLDELGHAEASMRVSDLIYEFMKKGDERLLEEAERLLEEVER

SEQ ID NO: 8 - protein, 56 aa, synthetic construct; identical to SEQ ID NO: 4
  SITE 1: note = Acetylated
  SITE 56: note = two PEG8 linkers
  SITE 56: note = Universal antibody binding terminus

SEQ ID NO: 9 - protein, 69 aa, synthetic construct
  SITE 56..57: note = 2 PEG8 linkers
  DISULFID 58..68: note = join(58,68)
  SITE 1: note = acetylated
DKEWILQKIYEIMRLLDELGHAEASMRVSDLIYEFMKKGDERLLEEAERLLEEVERDCAWHLGELVWCT

SEQ ID NO: 10 - protein, 64 aa, synthetic construct; identical to SEQ ID NO: 3
  SITE 64: note = pegylated linker and universal antibody binding terminus

SEQ ID NO: 11 - protein, 64 aa, synthetic construct; identical to SEQ ID NO: 3
  SITE 1: note = Acetylated
  SITE 64: note = 2 PEG8 linkers and universal antibody binding terminus

SEQ ID NO: 12 - protein, 39 aa, synthetic construct
  SITE 1: note = Acetylated
  MOD_RES 2: note = Alloc amino acid
  MOD_RES 8: note = Alloc amino acid
  MOD_RES 37: note = Alloc amino acid
  MOD_RES 38: note = Alloc amino acid
  SITE 1..39: note = sidechain protected
DKEWILQKIYEIMRLLDELGHAEASMRVSDLIYEFMKKG

SEQ ID NO: 13 - protein, 39 aa, synthetic construct; identical to SEQ ID NO: 12
  SITE 1: note = acetylated
  MOD_RES 2: note = Alloc amino acid
  MOD_RES 37: note = Alloc amino acid
  MOD_RES 38: note = Alloc amino acid
  SITE 39: note = tetrafluorobenzene

SEQ ID NO: 14 - protein, 13 aa, synthetic construct
  SITE 13: note = amidated C-terminus
  SITE 13: note = side chain protected
DCAWHLGELVWCT

SEQ ID NO: 15 - protein, 13 aa, synthetic construct
  DISULFID 2..12: note = 2,12 join
  SITE 13: note = amidated C-terminus
DCAWHLGELVWCT

SEQ ID NO: 16 - protein, 52 aa, synthetic construct
  MOD_RES 1: note = acetylated
  MOD_RES 2: note = Alloc amino acid
  MOD_RES 8: note = Alloc amino acid
  MOD_RES 38: note = Alloc amino acid
  MOD_RES 37: note = Alloc amino acid
  DISULFID 41..51: note = join 41,51
  MOD_RES 52: note = Amidated C-terminus
DKEWILQKIYEIMRLLDELGHAEASMRVSDLIYEFMKKGDCAWHLGELVWCT

SEQ ID NO: 17 - protein, 52 aa, synthetic construct; identical to SEQ ID NO: 16
  MOD_RES 1: note = acetylated
  DISULFID 41..51: note = 41,51 join
  MOD_RES 52: note = Amidated

SEQ ID NO: 18 - protein, 69 aa, synthetic construct; identical to SEQ ID NO: 9
  SITE 1: note = acetylated
  DISULFID 58..68: note = join 58,68

SEQ ID NO: 19 - protein, 70 aa, synthetic construct
  SITE 1: note = chloroacetic acid
  SITE 11: note = 1,2,3,4-tetrahydroisoquinoline-3-carboxylic acid
  SITE 12: note = Aib
  SITE 14: note = D amino acid
  SITE 70: note = side chain protected
ASTHLGELVWXXCEDKEWILQKIYEIMRLLDELGHAEASMRVSDLIYEFMKKGDERLLEEAERLLEEVER

SEQ ID NO: 20 - protein, 70 aa, synthetic construct; identical to SEQ ID NO: 19
  SITE 1: note = Acetylated
  SITE 11: note = 1,2,3,4-tetrahydroisoquinoline-3-carboxylic acid
  SITE 12: note = Aib
  SITE 14: note = D amino acid
  CROSSLNK 1..13: note = 1,13 join

SEQ ID NO: 21 - protein, 51 aa, synthetic construct
  SITE 1: note = Acetylated
  SITE 51: note = Sidechain protected
DCAWHLGELVWCTDKEWILQKIYEIMRLLDELGHAEASMRVSDLIYEFMKK

SEQ ID NO: 22 - protein, 51 aa, synthetic construct; identical to SEQ ID NO: 21
  SITE 1: note = Acetylated
  DISULFID 2..12: note = 2,12 join

SEQ ID NO: 23 - protein, 39 aa, synthetic construct
  SITE 39: note = Amidated
  DISULFID 16..25: note = 16,25 join
DKEWILQKIYEIMRLCDELGHAEACMRVSDLIYEFMKKG

SEQ ID NO: 24 - protein, 48 aa, synthetic construct
  SITE 1: note = acetylated
  MOD_RES 12: note = acetamidomethyl
  MOD_RES 21: note = acetomidomethyl
  MOD_RES 47: note = Trityl
  MOD_RES 37: note = Trityl
  SITE 48: note = amidated
  SITE 1..48: note = sidechain protected
ILQKIYEIMRLCDELGHAEACMRVSDLIYEFMKKGDCAWHLGELVWCT

SEQ ID NO: 25 - protein, 48 aa, synthetic construct; identical to SEQ ID NO: 24
  SITE 1: note = Acetylated
  MOD_RES 12: note = acetamidomethyl
  MOD_RES 21: note = acetamidomethyl
  SITE 48: note = amidated

SEQ ID NO: 26 - protein, 48 aa, synthetic construct; identical to SEQ ID NO: 24
  SITE 1: note = acetylated
  MOD_RES 12: note = acetamidomethy
  MOD_RES 21: note = acetamidomethyl
  DISULFID 37..47: note = 37,47 join
  SITE 48: note = amidated

SEQ ID NO: 27 - protein, 48 aa, synthetic construct; identical to SEQ ID NO: 24
  SITE 1: note = acetylated
  DISULFID 11..21: note = 11,21 join
  DISULFID 37..47: note = 37,47 join
  SITE 48: note = amidated

SEQ ID NO: 28 - protein, 77 aa, synthetic construct
  SITE 1: note = Acetylated
  MOD_RES 77: note = amidated
  MOD_RES 1..77: note = sidechain protected
DKWEILQKIYEIMRLLDELGHAEASMRVSDLIYEFMKKGDERLLEEAERLLEEVERGGGGGGGGDCAWHLGELVWCT

SEQ ID NO: 29 - protein, 77 aa, synthetic construct
  SITE 1: note = ACETYLATED
  MOD_RES 77: note = AMIDATED
DKEWILQKIYEIMRLLDELGHAEASMRVSDLIYEFMKKGDERLLEEAERLLEEVERGGGGGGGGDCAWHLGELVWCT

SEQ ID NO: 30 - protein, 77 aa, synthetic construct; identical to SEQ ID NO: 29
  SITE 1: note = acetylated
  MOD_RES 77: note = AMIDATED
  DISULFID 66..76: note = 66,76 JOIN

SEQ ID NO: 31 - protein, 62 aa, synthetic construct
  SITE 1: note = acetylated
  NON_TER 62: note = linked to universal antibody terminal peptide
DKEWILQKIYEIMRLLDELGHAEASMRVSDLIYEFMKKGDERLLEEAERLLEEVERGGGGGG

SEQ ID NO: 32 - protein, 60 aa, synthetic construct
  SITE 1: note = acetylated
  NON_TER 60: note = linked to uABT-NH2
DKEWILQKIYEIMRLLDELGHAEASMRVSDLIYEFMKKGDERLLEEAERLLEEVERGGGG

SEQ ID NO: 33 - protein, 13 aa, synthetic construct
  SITE 13: note = amidated
DCAWHLGELVWCT

SEQ ID NO: 34 - protein, 140 aa, synthetic construct
  SITE 1: note = chloroacetic acid
  SITE 11: note = 1,2,3,4-tetrahydroisoquinoline-3-carboxylic acid
  SITE 12: note = Aib
  SITE 14: note = D-Glutamic Acid
ASTHLGELVWXXCEDKEWILQKIYEIMRLLDELGHAEASMRVSDLIYEFMKKGDERLLEEAERLLEEVERASTHLGELVWXXCEDKEWILQKIYEIMRLLDELGHAEASMRVSDLIYEFMKKGDERLLEEAERLLEEVER

SEQ ID NO: 35 - protein, 51 aa, synthetic construct; identical to SEQ ID NO: 21
  SITE 1: note = acetylated

SEQ ID NO: 36 - protein, 35 aa, synthetic construct
  SITE 1: note = acetylated
  DISULFID 11..21: note = 11,21 join
  NON_TER 35: note = linked to Universal Antibody Binding Terminal with amidated C-terminus
ILQKIYEIMRLCDELGHAEACMRVSDLIYEFMKKG

SEQ ID NO: 37 - protein, 64 aa, synthetic construct
  SITE 1: note = acetylated
  NON_TER 64: note = linked to Universal Antibody Binding Terminal
DKEWILQKIYEIMRLLDELGHAEASMRVSDLIYEFMKKGDERLLEEAERLLEEVERGGGGGGGG

SEQ ID NO: 38 - protein, 71 aa, synthetic construct
  SITE 1: note = acetylated
  DISULFID 60..70: note = 60,70 join
  SITE 71: note = amidated
DKEWILQKIYEIMRLLDELGHAEASMRVSDLIYEFMKKGDERLLEEAERLLEEVERGGDCAWHLGELVWCT

SEQ ID NO: 39 - protein, 79 aa, synthetic construct
  SITE 1: note = Acetylated
  DISULFID 68..78: note = 68,78 join
  SITE 79: note = Amidated
DKEWILQKIYEIMRLLDELGHAEASMRVSDLIYEFMKKGDERLLEEAERLLEEVEREAAAKEAAAKDCAWHLGELVWCT

SEQ ID NO: 40 - protein, 77 aa, synthetic construct
  SITE 1: note = acetylated
  DISULFID 66..76: note = 66,76 join
  SITE 77: note = Amidated
DKEWILQKIYEIMRLLDELGHAEASMRVSDLIYEFMKKGDERLLEEAERLLEEVERGQAPGQAPDCAWHLGELVWCT

SEQ ID NO: 41 - protein, 37 aa, synthetic construct; identical to SEQ ID NO: 1

SEQ ID NO: 42 - protein, 37 aa, synthetic construct; identical to SEQ ID NO: 1
  SITE 1: note = acetylated
  SITE 1..37: note = fully protected

SEQ ID NO: 43 - protein, 39 aa, synthetic construct; identical to SEQ ID NO: 12
  SITE 1: note = acetylated
  SITE 2: note = alloc amino acid
  SITE 8: note = alloc amino acid
  SITE 37: note = alloc amino acid
  SITE 38: note = alloc amino acid
  SITE 39: note = tetrafluorobenzene

SEQ ID NO: 44 - protein, 13 aa, synthetic construct
  SITE 1: note = PEG-8 linked
  SITE 13: note = amidated
  SITE 1..13: note = sidechain protected
DCAWHLGELVWCT

SEQ ID NO: 45 - protein, 13 aa, synthetic construct
  SITE 1: note = PEG-8 linked
  SITE 13: note = amidated
DCAWHLGELVWCT

SEQ ID NO: 46 - protein, 13 aa, synthetic construct
  SITE 1: note = PEG-8 linked
  SITE 13: note = amidated
  DISULFID 2..12: note = 2,12 join
DCAWHLGELVWCT

SEQ ID NO: 47 - protein, 53 aa, synthetic construct
  SITE 1: note = acetylated
  SITE 2: note = Alloc amino acid
  SITE 8: note = Alloc amino acid
  SITE 37: note = Alloc amino acid
  SITE 38: note = Alloc amino acid
  SITE 40: note = PEG-8
  DISULFID 42..52: note = 42,52 join
  SITE 53: note = amidated
DKEWILQKIYEIMRLLDELGHAEASMRVSDLIYEFMKKGXDCAWHLGELVWCT

SEQ ID NO: 48 - protein, 53 aa, synthetic construct; identical to SEQ ID NO: 47
  SITE 1: note = acetylated
  SITE 40: note = PEG-8
  DISULFID 42..52: note = 42,52 join
  SITE 53: note = amidated

SEQ ID NO: 49 - protein, 39 aa, synthetic construct; identical to SEQ ID NO: 12
  SITE 1: note = Acetylated
  SITE 2: note = alloc amino acid
  SITE 8: note = alloc amino acid
  SITE 37: note = alloc amino acid
  SITE 38: note = alloc amino acid
  SITE 39: note = amidated
  SITE 1..39: note = side chain protected

SEQ ID NO: 50 - protein, 39 aa, synthetic construct; identical to SEQ ID NO: 12
  SITE 1: note = linked to PEG8 followed by tetrafluorobenzene
  SITE 2: note = Alloc amino acid
  SITE 8: note = Alloc amino acid
  SITE 37: note = Alloc amino acid
  SITE 38: note = Alloc amino acid
  SITE 39: note = amidated

SEQ ID NO: 51 - protein, 53 aa, synthetic construct
  DISULFID 2..12: note = 2,12 join
  SITE 14: note = dia-PEG8-acid
  SITE 16: note = alloc amino acid
  SITE 22: note = alloc amino acid
  SITE 51: note = alloc amino acid
  SITE 52: note = alloc amino acid
  SITE 53: note = amidated
DCAWHLGELVWCTXDKEWILQKIYEIMRLLDELGHAEASMRVSDLIYEFMKKG

SEQ ID NO: 52 - protein, 53 aa, synthetic construct; identical to SEQ ID NO: 51
  DISULFID 2..12: note = 2,12 join
  SITE 14: note = dia-PEG8-acid
  SITE 53: note = amidated

SEQ ID NO: 53 - protein, 39 aa, synthetic construct; identical to SEQ ID NO: 12
  SITE 1: note = acetylated
  SITE 2: note = alloc
  SITE 8: note = alloc
  SITE 37: note = alloc
  SITE 38: note = alloc
  NON_TER 39
  SITE 39: note = amidated

SEQ ID NO: 54 - protein, 41 aa, synthetic construct
  SITE 1: note = acetylated
  SITE 39: note = amidated
  NON_TER 39
ACDKEWILQKIYEIMRLLDELGHAEASMRVSDLIYEFMKKG

SEQ ID NO: 55 - protein, 39 aa, synthetic construct; identical to SEQ ID NO: 12
  SITE 1: note = acetylated
  NON_TER 1
  SITE 39: note = amidated; note = protected

SEQ ID NO: 56 - protein, 39 aa, synthetic construct; identical to SEQ ID NO: 12
  SITE 1: note = non-terminal; note = acetylated
  SITE 39: note = amidated

SEQ ID NO: 57 - protein, 33 aa, synthetic construct
  MOD_RES 33: note = linked to Universal Antibody Binding Terminal with amidated C-terminus
  MOD_RES 1: note = Acetylated
QKIYEIMRLCDELGHAEACMRVSDLIYEFMKKG

SEQ ID NO: 58 - protein, 81 aa, synthetic construct
  MOD_RES 1: note = acetylated
DKEWILQKIYEIMRLLDELGHAEASMRVSDLIYEFMKKGDERLLEEAERLLEEVERPASPASPASPASDCAWHLGELVWCT

SEQ ID NO: 59 - protein, 81 aa, synthetic construct; identical to SEQ ID NO: 58
  MOD_RES 1: note = Acetylated
  DISULFID 70..80: note = link 70, 80

SEQ ID NO: 60 - protein, 60 aa, synthetic construct
  MOD_RES 1: note = Acetylated
  MOD_RES 49..59: note = linked 49,59
DKEWILQKIYEIMRLLDELGHAEASMRVSDLIYEFMKKGGGGGGGGGDCAWHLGELVWCT

SEQ ID NO: 61 - protein, 64 aa, synthetic construct
  MOD_RES 1: note = Acetylated
  DISULFID 53..63: note = linked 53,63
DKEWILQKIYEIMRLLDELGHAEASMRVSDLIYEFMKKGPASPASPASPASDCAWHLGELVWCT

SEQ ID NO: 62 - protein, 39 aa, synthetic construct; identical to SEQ ID NO: 12
  MOD_RES 1: note = Acetylated
  MOD_RES 39: note = linked to PEG8-PEG8PEG8-universal antibody binding moiety

SEQ ID NO: 63 - protein, 77 aa, synthetic construct; identical to SEQ ID NO: 29
  MOD_RES 77: note = Amidated
  DISULFID 66..76: note = linked 66,76

SEQ ID NO: 64 - protein, 75 aa, synthetic construct
  MOD_RES 75: note = Amidated
  DISULFID 64..74: note = linked 64,74
DKEWILQKIYEIMRLLDELGHAEASMRVSDLIYEFMKKGDERLLEEAERLLEEVERGGGGGGDCAWHLGELVWCT

SEQ ID NO: 65 - protein, 73 aa, synthetic construct
  MOD_RES 73: note = Amidated
  DISULFID 62..72: note = linked 62,72
DKEWILQKIYEIMRLLDELGHAEASMRVSDLIYEFMKKGDERLLEEAERLLEEVERGGGGDCAWHLGELVWCT

SEQ ID NO: 66 - protein, 71 aa, synthetic construct; identical to SEQ ID NO: 38
  MOD_RES 71: note = Amidated
  DISULFID 60..70: note = linked 60,70

SEQ ID NO: 67 - protein, 79 aa, synthetic construct; identical to SEQ ID NO: 39
  MOD_RES 79: note = Amidated
  DISULFID 68..78: note = linked 68,78

SEQ ID NO: 68 - protein, 77 aa, synthetic construct; identical to SEQ ID NO: 40
  MOD_RES 77: note = Amidated
  DISULFID 66..76: note = linked 66,76

SEQ ID NO: 69 - protein, 39 aa, synthetic construct; identical to SEQ ID NO: 23
  MOD_RES 1: note = Acetylated

SEQ ID NO: 70 - protein, 39 aa, synthetic construct; identical to SEQ ID NO: 23
  MOD_RES 1: note = Acetylated
  MOD_RES 39: note = Amidated

SEQ ID NO: 71 - protein, 33 aa, synthetic construct; identical to SEQ ID NO: 57
  MOD_RES 1: note = Acetylated
  MOD_RES 33: note = Amidated
  DISULFID 10..19: note = linked 10,19

SEQ ID NO: 72 - protein, 33 aa, synthetic construct; identical to SEQ ID NO: 57
  MOD_RES 33: note = Amidated
  DISULFID 10..19: note = linked 10,19

SEQ ID NO: 73 - protein, 33 aa, synthetic construct; identical to SEQ ID NO: 57
  MOD_RES 1: note = Acetylated
  MOD_RES 33: note = Amidated
  DISULFID 10..19: note = linked 10,19

SEQ ID NO: 74 - protein, 35 aa, synthetic construct; identical to SEQ ID NO: 36
  MOD_RES 35: note = Amidated
  DISULFID 12..21: note = linked 12,21

SEQ ID NO: 75 - protein, 35 aa, synthetic construct; identical to SEQ ID NO: 36
  MOD_RES 1: note = Acetylated
  MOD_RES 35: note = Amidated
  DISULFID 12..21: note = linked 12,21

SEQ ID NO: 76 - protein, 39 aa, synthetic construct
  MOD_RES 1: note = linked to PEG4-biotin
DKEWILQKIYEICRLLDELGHAEASMRCSDLIYEFMKKG

SEQ ID NO: 77 - protein, 39 aa, synthetic construct; identical to SEQ ID NO: 76
  MOD_RES 1: note = linked to PEG4-biotin
  DISULFID 13..28: note = linked 13,28

SEQ ID NO: 78 - protein, 56 aa, synthetic construct; identical to SEQ ID NO: 4
  MOD_RES 56: note = Amidated

SEQ ID NO: 79 - protein, 64 aa, synthetic construct; identical to SEQ ID NO: 3
  MOD_RES 64: note = Amidated

SEQ ID NO: 80 - protein, 64 aa, synthetic construct; identical to SEQ ID NO: 3
  MOD_RES 1: note = linked to PEG4-biotin
  MOD_RES 64: note = Amidated

SEQ ID NO: 81 - protein, 56 aa, synthetic construct
  MOD_RES 1: note = linked to PEG4-biotin
  MOD_RES 56: note = Amidated
DKEWILQKIYEIMRLLDELGHAPASMRVSDLIYEFMKKGDERLLEEAERLLEEVER

SEQ ID NO: 82 - protein, 56 aa, synthetic construct
  MOD_RES 56: note = Amidated
  MOD_RES 20: note = D-histidine
DKEWILQKIYEIMRLLDELXHAPASMRVSDLIYEFMKKGDERLLEEAERLLEEVER

SEQ ID NO: 83 - protein, 56 aa, synthetic construct; identical to SEQ ID NO: 81
  MOD_RES 56: note = Amidated

SEQ ID NO: 84 - protein, 56 aa, synthetic construct
  MOD_RES 19: note = Norleucine
  MOD_RES 20: note = D-histidine
DKEWILQKIYEIMRLLDEXXHAEASMRVSDLIYEFMKKGDERLLEEAERLLEEVER

SEQ ID NO: 85 - protein, 56 aa, synthetic construct; identical to SEQ ID NO: 4
  MOD_RES 56: note = Amidated

SEQ ID NO: 86 - protein, 56 aa, synthetic construct; identical to SEQ ID NO: 4
  MOD_RES 1: note = Acetylated
  MOD_RES 56: note = Amidated

SEQ ID NO: 87 - protein, 64 aa, synthetic construct; identical to SEQ ID NO: 3
  MOD_RES 1: note = Acetylated
  MOD_RES 64: note = Amidated

SEQ ID NO: 88 - protein, 56 aa, synthetic construct; identical to SEQ ID NO: 82
  MOD_RES 20: note = D-histine
  MOD_RES 1: note = Acetylated
  MOD_RES 56: note = Amidated

SEQ ID NO: 89 - protein, 56 aa, synthetic construct; identical to SEQ ID NO: 84
  MOD_RES 19: note = Norleucine
  MOD_RES 20: note = D-histidine
  MOD_RES 1: note = Acetylated
  MOD_RES 56: note = Amidated

SEQ ID NO: 90 - protein, 56 aa, synthetic construct
  MOD_RES 56: note = D-alanine
DKEWILQKIYEIMRLLDELGHAEASMRVSDLIYEFMKKGDERLLEEAERLLEEVEX

SEQ ID NO: 91 - protein, 57 aa, synthetic construct
  MOD_RES 56: note = D-alanine
  MOD_RES 57: note = D-alanine
DKEWILQKIYEIMRLLDELGHAPASMRVSDLIYEFMKKGDERLLEEAERLLEEVEXX

SEQ ID NO: 92 - protein, 56 aa, synthetic construct
  MOD_RES 1: note = D-alanine
XKEWILQKIYEIMRLLDELGHAPASMRVSDLIYEFMKKGDERLLEEAERLLEEVER

SEQ ID NO: 93 - protein, 56 aa, synthetic construct; identical to SEQ ID NO: 4
  MOD_RES 1: note = Acetylated
  MOD_RES 56: note = linked to PEG8-PEG8-PEG2-COOH

SEQ ID NO: 94 - protein, 39 aa, synthetic construct
  MOD_RES 19: note = Norleucine
  MOD_RES 20: note = D-histidine
DKEWILQKIYEIMRLLDEXXHAEASMRVSDLIYEFMKKG

SEQ ID NO: 95 - protein, 39 aa, synthetic construct; identical to SEQ ID NO: 94
  MOD_RES 1: note = linked to PEG-biotin
  MOD_RES 19: note = Norleucine
  MOD_RES 20: note = D-histidine

SEQ ID NO: 96 - protein, 56 aa, synthetic construct; identical to SEQ ID NO: 90
  MOD_RES 56: note = D-alanine
  MOD_RES 1: note = Acetylated

SEQ ID NO: 97 - protein, 56 aa, synthetic construct
  MOD_RES 1: note = Acetylated
  MOD_RES 56: note = D-alanine
DKEWILQKIYEIMRLLDELGHAPASMRVSDLIYEFMKKGDERLLEEAERLLEEVEX

SEQ ID NO: 98 - protein, 39 aa, synthetic construct; identical to SEQ ID NO: 12
  MOD_RES 1: note = Acetylated
  MOD_RES 39: note = linked to PEG8-COOH

SEQ ID NO: 99 - protein, 40 aa, synthetic construct
  MOD_RES 1: note = 2-Aminoisobutyric acid (Aib); note = Acetylated
  MOD_RES 40: note = linked to PEG8-COOH
XDKEWILQKIYEIMRLLDELGHAEASMRVSDLIYEFMKKG

SEQ ID NO: 100 - protein, 20 aa, synthetic construct
  MOD_RES 1: note = Acetylated
  MOD_RES 20: note = linked to PEG8-COOH
DKEWILQKIYEIMRLLDELG

SEQ ID NO: 101 - protein, 39 aa, synthetic construct; identical to SEQ ID NO: 12
  MOD_RES 1: note = Acetylated
  MOD_RES 39: note = linked to PEG8-COOH

SEQ ID NO: 102 - protein, 29 aa, synthetic construct
  MOD_RES 1: note = Acetylated
  MOD_RES 29: note = linked to PEG8-COOH
DKEWILQKIYEIMRLLDELGHAEASMRVS

SEQ ID NO: 103 - protein, 35 aa, synthetic construct
  MOD_RES 1: note = Acetylated
  MOD_RES 35: note = linked to PEG8-COOH
ILQKIYEIMRLLDELGHAEASMRVSDLIYEFMKKG

SEQ ID NO: 104 - protein, 31 aa, synthetic construct
  MOD_RES 1: note = Acetylated
  MOD_RES 31: note = linked to PEG8-COOH
IYEIMRLLDELGHAEASMRVSDLIYEFMKKG

SEQ ID NO: 105 - protein, 39 aa, synthetic construct; identical to SEQ ID NO: 12
  MOD_RES 1: note = linked to PEG2-PEG2-PEG2-N3

SEQ ID NO: 106 - protein, 63 aa, synthetic construct
  MOD_RES 1: note = linked to PEG2-PEG2-NH2
  MOD_RES 63: note = Amidated
NDDELHMLMTDLVYEALHFKDEEIKKRVFQLFELADKAYKNNDRQKLEKVVEELKELLERLLS

SEQ ID NO: 107 - protein, 64 aa, synthetic construct; identical to SEQ ID NO: 3
  MOD_RES 1: note = linked to PEG2-PEG2-PEG8-PEG2-Reactive uABT
  MOD_RES 64: note = Amidated

SEQ ID NO: 108 - protein, 38 aa, synthetic construct
  MOD_RES 1: note = Acetylated
  MOD_RES 38: note = linkged to PEG8-COOH
DKEWILQKIYEIMRLLDELGHAEASMRVSDLIYEFMKK

SEQ ID NO: 109 - protein, 37 aa, synthetic construct
  MOD_RES 37: note = Homocystein
EWILQKIYEIMRLLDELGHAEASMRVSDLIYEFMKKX